(19) United States
(12) Patent Application Publication
(10) Pub. No.: US 2025/0129078 A1
(43) Pub. Date: Apr. 24, 2025

(54) ERBB2 INHIBITORS

(71) Applicant: Cogent Biosciences, Inc., Waltham, MA (US)

(72) Inventors: Leah J. Salituro, Boulder, CO (US); Jennifer Fulton, Boulder, CO (US); Ravi Kumar Jalluri, Boulder, CO (US); Logan E. Vine, Boulder, CO (US); Tanna Bettendorf, Boulder, CO (US); John Fischer, Boulder, CO (US); Jay B. Fell, Boulder, CO (US); Mark J. Chicarelli, Boulder, CO (US); Payal Chatterjee, Boulder, CO (US); Cori A. Malinky, Boulder, CO (US)

(21) Appl. No.: 18/632,187

(22) Filed: Apr. 10, 2024

Related U.S. Application Data
(60) Provisional application No. 63/495,543, filed on Apr. 11, 2023.

(51) Int. Cl. C07D 487/04; A61K 31/517; A61K 31/519; C07D 403/14; C07D 471/04; C07D 519/00

(52) U.S. Cl. CPC C07D 487/04; A61K 31/517; A61K 31/519; C07D 403/14; C07D 471/04; C07D 519/00

(57) ABSTRACT

A compound having the following structure of Formula (I):

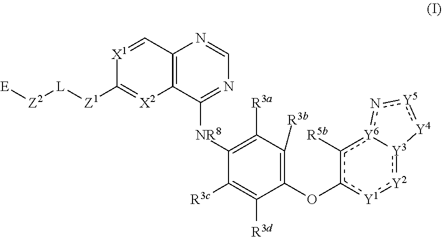

or a stereoisomer of the compound, pharmaceutically acceptable salt, or tautomer thereof, wherein E, L, R^{3a}, R^{3b}, R^{3c}, R^{3d}, R^{5b}, R⁸, Y¹, Y², Y³, Y⁴, Y⁵, Y⁶, X¹, X², Z¹, and Z² are as defined herein. Pharmaceutical composition comprising the compounds, and their use in methods of treating diseases are also described.

### TECHNICAL FIELD

The disclosure relates to substituted pyrimido pyrimidine compounds that act as a human epidermal growth factor receptor 2 (ErbB2) inhibitors. The disclosure also provides compounds of formula (I) and pharmaceutically acceptable salts thereof and uses of the compounds for the treatment of abnormal cell growth, such as cancer, in a subject.

### BACKGROUND

Human epidermal growth factor receptor 2 (ErbB2) is a member of the epidermal growth factor receptor family having tyrosine kinase activity. ErbB2 plays an important role in the signal transduction cascade in biochemical pathways responsible for cell growth and differentiation. It is known that alterations including amplifications and mutations of ErbB2 result in unregulated cell proliferation in cancers such as breast, bladder, colorectal, and lung. Existing therapies, including but not limited to monoclonal antibodies, bispecific antibodies, and kinase inhibitors, still pose high recurrence and acquired resistance issues.

While current kinase inhibitors can be effective treatments for cancer, their use may be limited by poor selectivity for ErbB2 over EGFR. EGFR related toxicities including GI effects and rash require regimen modification such as dosing holidays or dose reductions leading to suboptimal target coverage and reduced efficacy.

In addition ErbB2 exon 20 YVMA insertion is associated with a high incidence of brain metastasis in NSCL cancer patients with ErbB2 alterations. A brain penetrant ErbB2 inhibitor that is active against ErbB2 mutations including YVMA could be useful for treatment of patients with brain metastases.

There remains a need to identify potent, brain penetrant, mutant active, EGFR sparing, ErbB2 inhibitors for the treatment of patients with cancer or other proliferative diseases or conditions driven by ErbB2 alterations.

### BRIEF SUMMARY

In brief, the present disclosure provides compounds, including stereoisomers, tautomers, or pharmaceutically acceptable salts thereof, which can be used alone or in combination with other therapeutic agents.

In one embodiment, a compound having a structure of Formula (I) is provided:

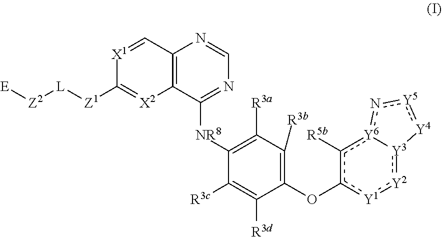

or a stereoisomer, tautomer, or pharmaceutically acceptable salt thereof, E, L, R^{3a}, R^{3b}, R^{3c}R^{3d}, R^{5b}, R⁸, Y¹, Y², Y³, Y⁴, Y⁵, Y⁶, X¹, X², Z¹, and Z² are as defined herein.

Pharmaceutical compositions comprising one or more of the foregoing compounds of Formula (I) and an additional therapeutic agent are also provided.

In other embodiments, methods of treatment by administering the foregoing compounds of Formula (I) or the pharmaceutical compositions comprising a compound of Formula (I), to a subject in need thereof to treat a disease are provided.

Various aspects and embodiments now will be described more fully hereinafter. Such aspects and embodiments may take many different forms, and the exemplary ones disclosed herein should not be construed as limiting; rather, these embodiments are provided so that this disclosure will be thorough and complete, and will fully convey its scope to those skilled in the art.

### DETAILED DESCRIPTION

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 mM to 8 mM is stated, it is intended that 2 mM, 3 mM, 4 mM, 5 mM, 6 mM, and 7 mM are also explicitly disclosed, as well as the range of non-integer values greater than or equal to 1 mM and the range of non-integer values less than or equal to 8 mM.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the compositions and all measurements made are at about 25° C., unless otherwise specified. As used in the specification and appended claims, unless specified to the contrary, the following terms have the meaning indicated:

“Amino” refers to the —NH₂, —NHR, or —NR₂ radical,

“Cyano” refers to the —CN radical,

“Hydroxyl” refers to the —OH radical,

“Imino” refers to the =NH or =NR substituent,

“Nitro” refers to the —NO2 radical,

“Oxo” refers to the =O substituent,

“Thio” refers to the =S substituent,

“Trifluoromethyl” refers to the —CF₃ radical,

Hydrazido or hydrazino refers to N—N substituent,

wherein each R of “amino” or “imino” is a compatible substituent as described in this disclosure and wherein an R group is chiral, isomers are contemplated and included herein.

“Alkyl” refers to a linear, saturated, acyclic, monovalent hydrocarbon radical or branched, saturated, acyclic, monovalent hydrocarbon radical, having from one to twelve carbon atoms, preferably one to eight carbon atoms or one to six carbon atoms, and which is attached to the rest of the molecule by a single bond, e.g., methyl, ethyl, n-propyl, 1-methylethyl (isopropyl), n-butyl, n-pentyl, 1,1-dimethylethyl (t-butyl), 3-methylhexyl, 2-methylhexyl and the like. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilyl, —OR′, —OC(O)R′, —N(R′)₂, C(O)R″, —C(O)OR′, —C(O)N(R′)₂, —N(R′)C(O)OR′″, N(R′)C(O)R′″, —N(R′)S(O)ₜR′″(where t is 1 or 2), —S(O)ₜOR′″(where t is 1 or 2), —S(O)ₚR′″(where p is 0, 1, or 2) and —S(O)ₜN(R′)₂ (where t is 1 or 2), where each R′ is independently hydrogen, alkyl, haloalkyl, cycloalkyl, cycloalkylalkyl, aryl, aralkyl, heterocyclyl, or heteroaryl; each R″ is independently hydrogen, cycloalkyl, aryl, heterocyclyl, or heteroaryl; and each R′″ is independently alkyl, haloalkyl, cycloalkyl, aryl, heterocyclyl, or heteroaryl.

“Alkoxy” refers to a radical of the formula —ORₐ where Rₐ is an alkyl radical as defined above containing one to twelve carbon atoms. The alkyl part of the optionally substituted alkoxy radical is optionally substituted as defined above for an alkyl radical.

“Alkoxyalkyl” refers to a radical of the formula —Rₐ—O—R_{b} where Rₐ is alkylene and R_{b} is alkyl as defined above. Alkyl and alkylene parts of the optionally substituted alkoxyalkyl radical are optionally substituted as defined above for an alkyl radical and alkylene chain, respectively.

“Aralkyl” refers to a radical of the formula —Rₐ—R_{b}, where Rₐ is alkylene and R_{b} is aryl as described herein. Alkylene and aryl portions of optionally substituted aralkyl are optionally substituted as described herein for alkylene and aryl, respectively.

“Aryl” refers to an aromatic monocyclic or multicyclic hydrocarbon ring system radical containing from 6 to 18 carbon atoms, where the multicyclic aryl ring system is a bicyclic, tricyclic, or tetracyclic ring system. Aryl radicals include, but are not limited to, groups such as fluorenyl, phenyl and naphthyl. An optionally substituted aryl is an aryl radical that is optionally substituted by one, two, three, four, or five substituents independently selected from the group consisting of alkyl, akenyl, halo, haloalkyl, haloalkenyl, cyano, nitro, aryl, heteroaryl, heteroarylalkyl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₚR′″(where p is 0, 1, or 2), and —R″—S(O)ₜN(R′)₂ (where t is 1 or 2), where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, heterocyclyl, or heteroaryl.

“Arylalkoxy” refers to a group of formula —O—R, where R is aralkyl. An optionally substituted arylalkoxy is an arylalkoxy that is optionally substituted as described herein for aralkyl. In some embodiments, arylalkoxy is benzyloxy.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₚR′″(where p is 0, 1, or 2) and —R″—S(O)ₜN(R′)₂ (where t is 1 or 2) where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, aralkyl, heterocyclyl, or heteroaryl.

“Deuterated compounds” are compounds where one or more hydrogen atoms have been replaced with a deuterium atom. Deuterated drugs may be derivatives of an active compound. Deuterated drugs may be prodrugs. Deuteration may alter the physical properties, metabolic properties, activity or safety of a drug.

“Derivatives” are related chemical species that can be derived from a similar compound via chemical reactions. They may encompass slight chemical modifications, substitution of atoms with deuterated atoms, substitution of atoms with stable or radioactive isotopes or other modifications that imbue a compound with desirable properties.

“Fused” refers to any ring system described herein which is fused to an existing ring structure in the compounds of the invention. When the fused ring system is a heterocyclyl or a heteroaryl, any carbon atom on the existing ring structure which becomes part of the fused ring system may be replaced with a nitrogen atom.

“Halo” refers to the halogen substituents: bromo, chloro, fluoro, and iodo.

“Haloalkyl” refers to an alkyl radical, as defined above, that is further substituted by one or more halogen substituents. The number of halo substituents included in haloalkyl is from one and up to the total number of the hydrogen atoms available for replacement with the halo substituents (e.g., perfluoroalkyl). Non-limiting examples of haloalkyl include trifluoromethyl, difluoromethyl, trichloromethyl, 2,2,2-trifluoroethyl, 1-fluoromethyl, 2-fluoroethyl, 3-bromo 2-fluoropropyl, 1-bromomethyl, 2-bromoethyl and the like. For an optionally substituted haloalkyl, the hydrogen atoms bonded to the carbon atoms of the alkyl part of the haloalkyl radical may be optionally replaced with substituents as defined above for an optionally substituted alkyl.

“Haloalkenyl” refers to an alkenyl radical, as defined above, that is further substituted by one or more halo substituents. The number of halo substituents included in haloalkenyl is from one and up to the total number of the hydrogen atoms available for replacement with the halo substituents (e.g., perfluoroalkenyl). Non-limiting examples of haloalkenyl include 2,2-difluoroethenyl, 3-chloroprop-1-enyl, and the like. For an optionally substituted haloalkenyl, the hydrogen atoms bonded to the carbon atoms of the alkenyl part of the haloalkenyl radical may be optionally replaced with substituents as defined above for an optionally substituted alkenyl group.

“Haloalkynyl” refers to an alkynyl radical, as defined above, that is further substituted by one or more halo substituents. The number of halo substituents included in haloalkynyl is from one and up to the total number of the hydrogen atoms available for replacement with the halo substituents (e.g., perfluoroalkynyl). Non-limiting examples of haloalkynyl include 3-chloroprop-1-ynyl and the like. The alkynyl part of the haloalkynyl radical may be additionally optionally substituted as defined above for an alkynyl group.

“Heteroarylalkyl” refers to a radical of the formula —Rₐ—R_{b}, where Rₐ is alkylene and R_{b} is heteroaryl as described herein. Alkylene and heteroaryl portions of optionally substituted heteroarylalkyl are optionally substituted as described herein for alkylene and heteroaryl, respectively.

“Heterocyclyl” refers to a stable 3- to 18-membered nonaromatic ring system radical having the carbon count of two to twelve and containing a total of one to six heteroatoms independently selected from the group consisting of nitrogen, oxygen, phosphorus, and sulfur. A heterocyclyl radical is a monocyclic, bicyclic, tricyclic, or tetracyclic ring system. A bicyclic, tricyclic, or tetracyclic heterocyclyl is a fused, spiro, and/or bridged ring system. The heterocyclyl radical may be saturated or unsaturated. An unsaturated heterocyclyl contains one, two, or three carbon-carbon double bonds and/or one carbon-carbon triple bond. An optionally substituted heterocyclyl is a heterocyclyl radical that is optionally substituted by one, two, three, four, or five substituents independently selected from the group consisting of alkyl, alkenyl, halo, haloalkyl, haloalkenyl, cyano, oxo, thioxo, nitro, aryl, aralkyl, cycloalkyl, heterocyclyl-, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₚR′″(where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is independently alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom); the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₚR′″(where p is 0, 1, or 2), and —R″—S(O)ₜN(R′)₂ (where t is 1 or 2), where R″ is a linear or branched alkylene or alkenylene chain, and R′ and R′″ are as defined above). Examples of optionally substituted heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2oxopyrrolidinyl, oxazolidinyl-, piperidinyl, piperazinyl, 4piperidonyl, pyrrolidinyl, pyrazolidinyl, thiazolidinyl, tetrahydrofuryl, trithianyl, tetrahydropyranyl, thiomorpholinyl, thiamorpholinyl, 1-oxothiomorpholinyl, and 1,1-dioxo-thiomorpholinyl.

“Heterocyclylene” refers to a heterocyclyl in which one hydrogen atom is replaced with a valency. An optionally substituted heterocyclylene is optionally substituted as described herein for heterocyclyl.

“Heteroaryl” refers to a 5- to 18-membered ring system radical containing at least one aromatic ring, having the carbon count of one to seventeen carbon atoms, and containing a total of one to ten heteroatoms independently selected from the group consisting of nitrogen, oxygen, and sulfur. The heteroaryl radical is a monocyclic, bicyclic, tricyclic, or tetracyclic ring system. The bicyclic, tricyclic, or tetracyclic heteroaryl radical is a fused and/or bridged ring system. An optionally substituted heteroaryl is a heteroaryl radical that is optionally substituted by one, two, three, four, or five substituents independently selected from the group consisting of alkyl, alkenyl, alkoxy, halo, haloalkyl, haloalkenyl, cyano, oxo, thioxo, nitro, oxo, aryl, aralkyl, cycloalkyl, heterocyclyl, heteroaryl, or heteroarylalkyl-, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₜR′″(where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom), provided that at least one ring in heteroaryl remains aromatic; the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″(where t is 1 or 2), —R″—S(O)ₜOR′″(where t is 1 or 2), —R″—S(O)ₚR′″(where p is 0, 1, or 2), and —R″—S(O)ₜN(R′)₂ (where t is 1 or 2), where R″ is a linear or branched alkylene or alkenylene chain, and R′ and R′″ are as defined above), provided that at least one ring in heteroaryl remains aromatic. Examples of optionally substituted heteroaryl radicals include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo-[1,2a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl-, 2oxoazepinyl, oxazolyl, oxiranyl-, 1-phenyl-1Hpyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl- (i.e., thienyl).

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in animals (e.g., mammals), and more particularly, in humans.

“Prodrugs” are compounds that after administration are metabolized or otherwise chemically transformed into an active moiety. Prodrugs may be derivatives of an active compound. Prodrugs may or may not be active prior to conversion into an active form in vivo.

The term “treating” is used herein, for instance, in reference, for example, to methods of treating inflammatory diseases or to a gastrointestinal disease, and generally includes the administration of a compound or composition which reduces the frequency of, or delays the onset of, symptoms of a medical condition (e.g., autoimmune disease, inflammatory disorder, gastrointestinal disorder) in a subject relative to a subject not receiving the compound or composition. This can include reversing, reducing, or arresting the symptoms, clinical signs, and underlying pathology of a condition in a manner to improve or stabilize a subject's condition (e.g., regression of symptoms of an autoimmune or inflammatory disease such as improvement in the MAYO score in the treatment of ulcerative colitis).

The embodiments disclosed herein encompass all pharmaceutically acceptable compounds of the compound of (I)—(IL) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labelled compounds of (I)—(IL), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of (I)—(IL) can generally be prepared by conventional techniques known to those skilled in the art or by processes analogous to those described in the Preparations and Examples as set out below using an appropriate isotopically-labeled reagent in place of the non-labeled reagent previously employed.

The embodiments disclosed herein encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents and excipients therefor.

“Effective amount” or “therapeutically effective amount” refers to that amount of a compound of the disclosure which, when administered to a mammal, preferably a human, is sufficient to effect treatment in the mammal, preferably a human. The amount of a compound which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. The present disclosure also contemplates “diastereomers”, which refers to non-mirror image of non-identical stereoisomers. Diastereomers occur when two or more stereoisomers of a compound have different configurations at one or more of the equivalent stereocenters and are not mirror images of each other.

A “tautomer” refers to a proton shift from one atom of a molecule to another atom of the same molecule. The present disclosure includes tautomers of any said compounds.

“Abnormal cell growth”, as used herein, unless otherwise indicated, means cell growth that is independent of normal regulatory mechanisms (e.g., loss of contact inhibition). Abnormal cell growth may be benign (not cancerous) or malignant (cancerous).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Compounds

The compounds described herein are ErbB2 inhibitors. In one embodiment, a compound having a structure of Formula (I), or a stereoisomer, tautomer of the compound, or a pharmaceutically acceptable salt thereof is provided:

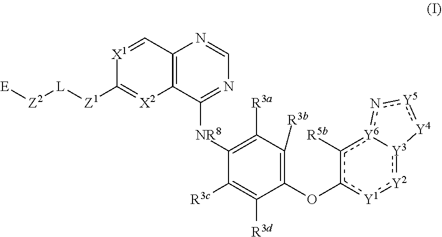

wherein X¹ is CR¹ or N; X² is CR² or N; Y¹ is CR⁴ or N; Y² is CR⁵ or N; Y³ is C or N; Y⁴ is CR⁶ or NR⁶; Y⁵ is CR⁷, O, or NR⁷; Y⁶ is C or N; R¹, R², R^{3a}, R^{3b}, R^{3c}, R^{3d}, R⁴, R^{5a}, R^{5b} and R⁷ are each independently H, halo, C₁-C₃ alkyl, C₁-C₃ haloalkyl, or C₁-C₃ heteroalkyl; R⁶ is H, C₁-C₃ alkyl, C₁-C₃ deutero-alkyl, or C₁-C₃ haloalkyl; R⁸ and R⁹ are, each independently, H or C₁-C₃ alkyl; Z¹ is —NR⁹—, —O—, —S—, or a direct bond; Z² is —NR⁹— or a direct bond; L is C₁-C₄ alkyl, C₃-C₈ cycloalkyl, C₃-C₈ heterocycloalkyl, a fused heterobicyclic, a bridged heterobicyclic, a hetero-spirocyclic, or a direct bond; E is α,β-unsaturated carbonyl or C₂-C₄ alkyne conjugated carbonyl; and [symbol] is, each independently, a single bond or a double bond, where valency permits.

In one embodiment, Y² is CR^{5a} or N. In some embodiments, Y² is CR^{5a}. In some embodiments, Y² is N.

In one embodiment, Y⁴ is CR⁶ or NR⁶. In some embodiments, Y⁴ is CR⁶. In some embodiments, Y⁴ is NR⁶.

In one embodiment, Y⁶ is C or N. In some embodiments, Y⁶ is C. In some embodiments, Y⁶ is N.

In one embodiment, Y² is CR^{5a}, Y⁴ is NR⁶, and Y⁶ is C. In one embodiment, Y² is N, Y⁴ is NR⁶, and Y⁶ is C. In one embodiment, Y² is CR^{5a}, Y⁴ is CR⁶, and Y⁶ is C. In one embodiment, Y² is N, Y⁴ is CR⁶, and Y⁶ is C.

In one embodiment, the compound has one of the following structures of Formula (IA)-(ID):

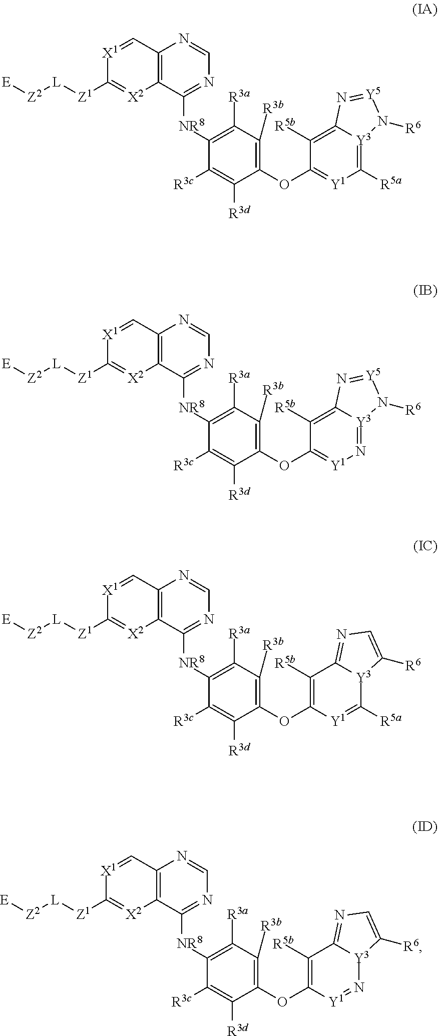

or a stereoisomer of the compound, tautomer of the compound, or a pharmaceutically acceptable salt thereof. In some embodiments, the compound has the structure of Formula (IA):

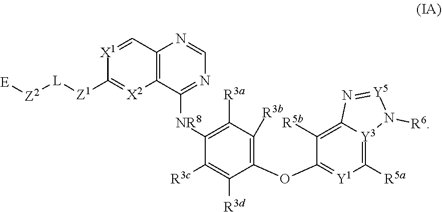

In some embodiments, the compound has the structure of Formula (IB):

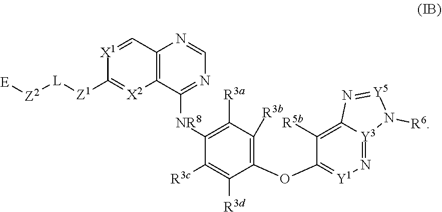

In some embodiments, the compound has the structure of Formula (IC):

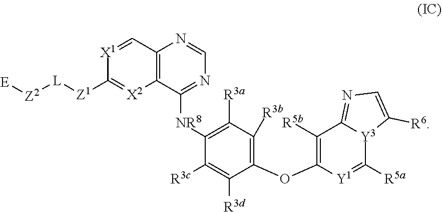

In some embodiments, the compound has the structure of Formula (ID):

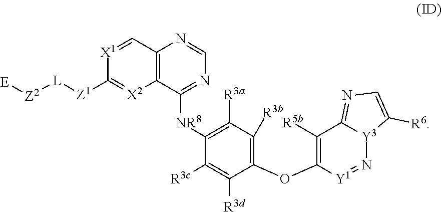

In one embodiment, Y⁵ is CR⁷, O, or NR⁷. In some embodiments, Y⁵ is CR⁷. In some embodiments, Y⁵ is O. Y⁵ is NR⁷.

In one embodiment, Y³ is C or N. In some embodiments, Y³ is C. In some embodiments, Y³ is N.

In one embodiment, Y⁵ is N and Y³ is C.

In one embodiment, the compound has one of the following structures of Formula (IA-1)-(IB-1):

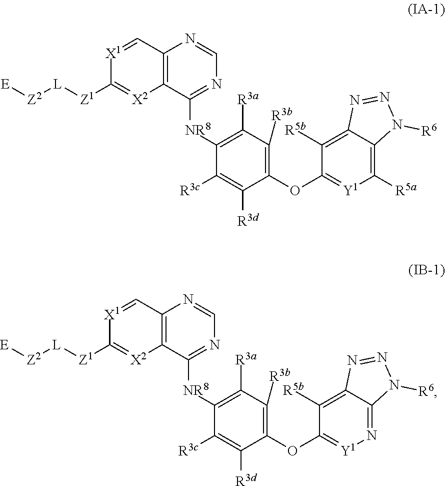

or a stereoisomer of the compound, tautomer of the compound, or a pharmaceutically acceptable salt thereof. In some embodiments, the compound has the structure of (IA-1):

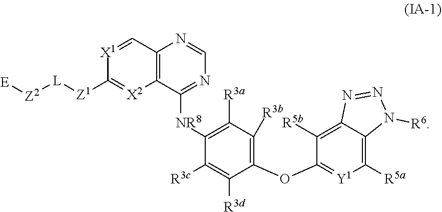

In some embodiments, the compound has the structure of (IB-1):

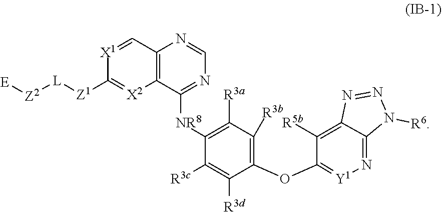

In one embodiment, Y⁵ is CR⁷, and Y³ is C, and R⁷ is H.

In one embodiment, the compound has one of the following structures of Formula (IA-2)-(IB-2):

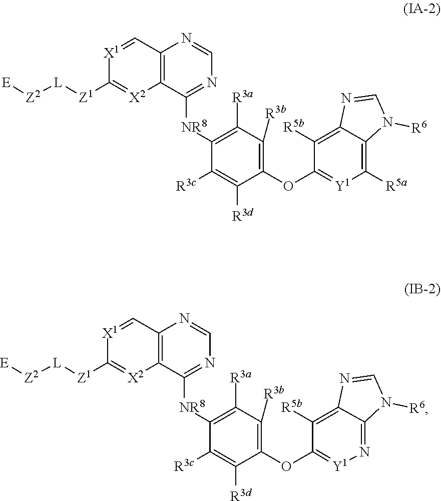

or a stereoisomer of the compound, tautomer of the compound, or a pharmaceutically acceptable salt thereof. In some embodiments, the compound has the structure of (IA-2):

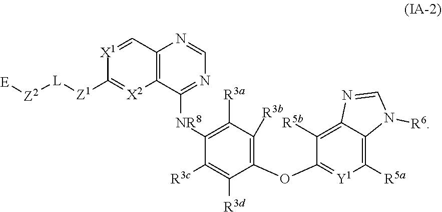

In some embodiments, the compound has the structure of (IB-2):

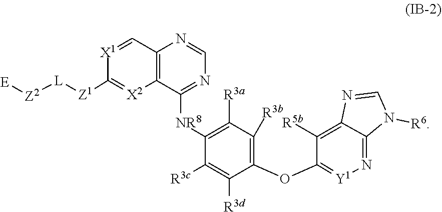

In one embodiment, R⁶ is H. C₁-C₃ alkyl, C₁-C₃ deutero-alkyl, or C₁-C₃ haloalkyl. In sorne embodiments, R⁶ is 1. In some embodiments, R⁶ is C₁-C₃ alkyl. In some embodiments, R⁶ is C₁-C₃ deutero-alkyl. In some embodiments, R⁶ is C₁-C₃ haloalkyl. In some certain embodiments, R⁶ is C₁ haloalkyl. In some more certain embodiments, R⁶ is CHF₂ or CF₃. In some embodiments, R⁶ is CHF₂. In some embodiments, R⁶ is CF₃.

In one embodiment, R⁶ of structures of (I), (IA)-(ID), (IA-1). (IA-2), (IB-1), or (IB-2) is C¹-C₃ haloalkyl. In some embodiments, R⁶ of Structures of (I), (IA)-(ID), (IA-1), (IA-2), (IB-1), or (IB-2) is C₁ haloalkyl. In some certain embodiments. R⁶ of structures of (I), (IA)-(ID), (IA-1), (IA-2), (IB-1), or (IB-2) is CHF₂ or CF₃, In some certain embodiments, R⁶ of structures of (I), (IA)-(ID), (IA-1), (IA-2), (IB-1), or (IB-2) is CHF₂. In some certain embodiments, R⁶ of structures of (I), (IA)-(D), (IA-1), (IA-2), (IB-1), or (IB-2) is CF₃,

In one embodiment, R⁶ of structures of (IA-1), (IA-2), or (IB-2) is C₁-C₃ haloalkyl. In some embodiments, R⁶ of structures of (IA-1), (IA-2), or (IB-2) is C₁ haloalkyl. In some certain embodiments, R⁶ of structures of (IA-1), (IA-2), or (IB-2) is CHF₂ or CF₃. In some certain embodiments, R⁶ of structures of (IA-1), (IA-2), or (IB-2) is CH F₂. In some certain embodiments, R⁶ of structures of (IA-1), (IA-2), or (IB-2) is CF₃.

In one embodiment, Z¹ is —NR⁹—, —O—, —S—, or a direct bond. In some embodiments, Z¹ is —NR⁹—. In some embodiments, Z¹ is —O—. In some embodiments, Z¹ is —S—. In some embodiments, Z¹ is a direct bond.

In one embodiment, Z² is —NR⁹— or a direct bond. In some embodiments, Z² is —NR⁹—. In some embodiments, Z¹ is a direct bond.

In one embodiment, Z¹ and Z² are each a direct bond. In this regard, the compound has the following structure:

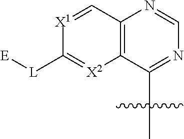

wherein the [symbol] represents the connection to the rest of the structures of (I), (IA)-(ID), (IA-1), (IA-2), (IB-1), or (IB-2).

In one embodiment, Z¹ is —NR⁹—, —O—, or —S— and Z² is a direct bond. In this regard, the compound has the following structure:

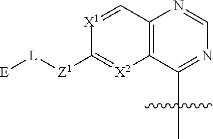

wherein the [symbol] resents the connection to the rest of the structures of (I), (IA)-(ID), (IA-1), (IA-2), (IB-1), or (IB-2).

In one embodiment, Z¹ is a direct bond and Z² is —NR⁹—. In this regard, the compound has the following structure:

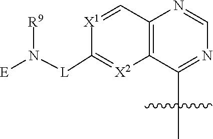

wherein the [symbol] represents the connection to the rest of the structures of (I), (IA)-(ID), (IA-1), (IA-2), (IB-1), or (IB-2).

In one embodiment, Z¹ and Z² are each —NR⁹—. In this regard, the compound has the following structure:

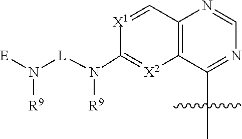

wherein the [symbol] represents the connection to the rest of the structures of (I), (IA)-(ID), (IA-I), (IA-2), (IB-1), or (IB-2).

In one embodiment, R⁸ and R⁹ are, each independently, H or C₁-C₃ alkyl. In one embodiment, R⁸ is H or C₁-C₃ alkyl. In some embodiments, R⁸ is H. In some embodiments, R⁸ is C₁-C₃ alkyl. In one embodiment, R⁹ is H or C₁-C₃ alkyl. In some embodiments, R⁹ is H. In some embodiments, R⁹ is C₁-C₃ alkyl. In some embodiments, R⁹ is H or C₁ alkyl. In some certain embodiments, R⁹ is H or C₁-C₃. In some embodiments, R⁹ is H. In some embodiments, R⁹ is CH₃.

In one embodiment, R¹, R², R^{3a}, R^{3b}, R^{3c}, R^{3d}, R⁴, R^{5a}, R^{5b}, and R⁷ are each independently H, halo, C₁-C₃ alkyl, C₁-C₃ haloalkyl, or C₁-C₃ heteroalkyl. In some embodiments, R¹, R², R^{3a}, R^{3b}, R^{3c}, R^{3d}, R⁴, R^{5a}, R^{5b}, and R⁷ are each independently H, halo, C₁ alkyl, C₁-C₂ haloalkyl, or C₁-C₃ heteroalkyl. In some embodiments, R¹, R², R^{3a}, R^{3b}, R^{3c}, R^{3d}, R⁴, R^{5a}, R^{5b}, and R⁷ are each independently H, F, Cl, CH₃, CH₂CF₃, CF₃, CHF₂, CH₂OCF₃, CD₃, OCHF₂, OCF₃, or OCH₃.

In one embodiment, R¹ and R² are, each independently, H, F, CH₃, or OCH₃. In some embodiments, R¹ is H, F, CH₃, or OCH₃. In some embodiments, R¹ is H. In some embodiments, R¹ is F. In some embodiments, R¹ is CH₃. In some embodiments, R¹ is OCH₃. In some embodiments, R² is H, F, CH₃, or OCH₃. In some embodiments, R² is H. In some embodiments, R² is F. In some embodiments, R² is CH₃. In some embodiments, R² is OCH₃.

In one embodiment, R^{3a}, R^{3b}, R^{3c}, and R^{3d} are, each independently, —H, CH₃, OCH₃, CH₂OCH, OCHF₂, OCF₃, F, or Cl. In some embodiments, R^{3a} is H, CH₃, OCH₃, CH₂OC₃, OCH—F₂, OCF₃, F, or Cl. In some embodiments, R^{3a} is H. In some embodiments, R^{3a} is CH₃. In some embodiments, R^{3a} is OCH₃. In some embodiments, R^{3a} is CH₂OCH₃. In some embodiments, R^{3a} is OCHF₂. In some embodiments, R^{3a} is OCF₃. In some embodiments, R^{3a} is F. In some embodiments, R^{3a} is Cl. In some embodiments, R^{3b} is H. In some embodiments, R^{3b} is CH₃. In some embodiments, R^{3b}, is OCH₃. In some embodiments, R^{3b} is CH₂OCH₃. In some embodiments, R^{3b} is OCHF₂. In some embodiments, R^{3b} is OCF₃. In some embodiments, R^{3b} is F. In some embodiments, R^{3b} is Cl. In some embodiments, R^{3c} is H. In some embodiments, R^{3c} is CH₃. In some embodiments, R^{3c} is OCH₃. In some embodiments, R^{3c} is CH₂OCH₃. In some embodiments, R^{3c} is OCHF₂. In some embodiments, R^{3c} is OCF₃. In some embodiments, R^{3c} is F. In some embodiments, R^{3c} is Cl. In some embodiments, R^{3d} is 1-1. In some embodiments. R^{3d} is CH₃. In some embodiments, R^{3d} is OCH₃. In some embodiments, R^{3d} is CH₂OCH₃. In some embodiments, R^{3d} is OCHF₂. In some embodiments, R^{3d} is OCF₃. In some embodiments, R^{3d} is F. In some embodiments. R^{3d} is Cl.

In one embodiment, R⁴ is H, F, CH₃, CH₂OCH₃, or CH₂CF₃. In some embodiments, R⁴ is H. In some embodiments, R⁴ is F. In some embodiments, R⁴ is CH₃. In some embodiments, R⁴ is CH₂OCH₃. In some embodiments, R⁴ is CH₂CF₃.

In one embodiment, R^{5a} and R^{5b} are, each independently, H, CH₃, F, or OCH₃. In some embodiments, R^{5a} is H, CH₃, F, or OCH₃. In some embodiments, R^{5a} is H. In some embodiments, R^{5a} is CH₃. In some embodiments, R^{5a} is F. In some embodiments, R^{5a} is OCH₃. In some embodiments, R^{5b} is H, CH₃, F, or OCH₃. In some embodiments, R^{5b} is H. In some embodiments, R^{5b} is CH₃. In some embodiments, R^{5b} is F. In some embodiments, R^{5b} is OCH₃.

In one embodiment, R⁷ is H or C₁ alkyl. In some embodiments, R⁷ is H. In some embodiments, R⁷ is C₁ alkyl. In some embodiments, R⁷ is H or CH₃, In some embodiments, R⁷ is CH₃.

In one embodiment, R⁸ is H or C₁ alkyl. In some embodiments, R⁸ is H. In some embodiments, R⁸ is C₁ alkyl. In some embodiments, R⁸ is H or CH₃. In some embodiments, R⁵ is CH₃.

In one embodiment,

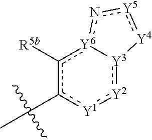

of the compound has one of the following structures:

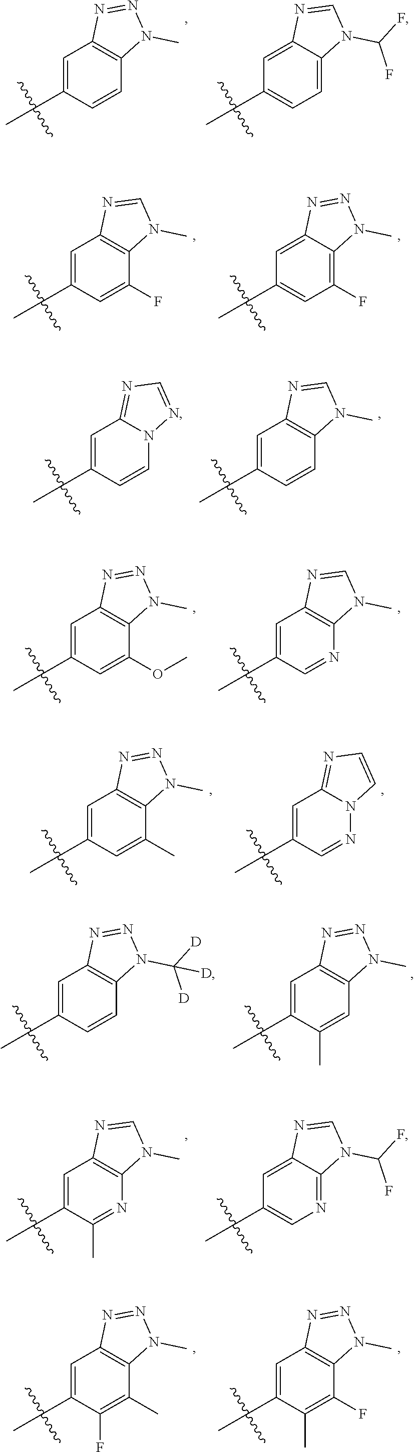

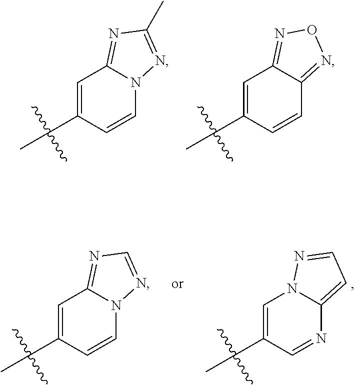 [SMILES: *c1ccn2nc(C)nc2c1 ; *c1ccc2nonc2c1 ; *c1ccn2ncnc2c1 ; *c1cnc2ccnn2c1]

wherein the [symbol] represents the connection to the rest of the structures of (I), (IA)-(ID), (IA-1), (IA-2). (IB-1), or (IB-2). In some embodiments, the compound has

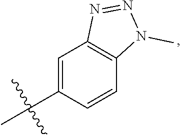 [SMILES: *c1ccc2c(c1)nnn2C]

wherein the [symbol] represents the connection to the rest of the structures of (I), (IA)-(ID), (IA-1), (IA-2), (IB-1), or (IB-2). In some embodiments, the compound has

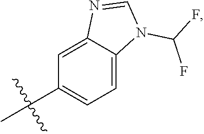 [SMILES: *c1ccc2c(c1)ncn2C(F)F]

wherein the [symbol] represents the connection to the rest of the structures of (I), (IA)-(ID), (IA-1) (IA-2), (IB-1), or (IB-2). In some embodiments, the compound has

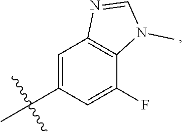 [SMILES: *c1cc(F)c2c(c1)ncn2C]

wherein the [symbol] represents the connection to the rest of the structures of (I), (IA)-(ID), (IA-1), (IA-2), (IB-1), or (IB-2). In some embodiments, the compound has

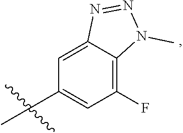 [SMILES: *c1cc(F)c2c(c1)nnn2C]

wherein the [symbol] represents the connection to the rest of the structures of (I), (IA)-(ID), (IA-1), (IA-2), (IB-1), or (IB-2). In some embodiments, the compound has

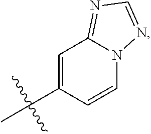 [SMILES: *c1ccn2ncnc2c1]

wherein the [symbol] represents the connection to the rest of the structures of (I), (IA)-(ID), (IA-1), (IA-2), (IB-1), or (IB-2). In some embodiments, the compound has

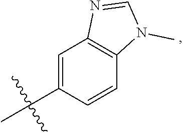 [SMILES: *c1ccc2c(c1)ncn2C]

wherein the [symbol] represents the connection to the rest of the structures of (I), (IA)-(ID), (IA-1), (IA-2), (IB-1), or (IB-2). In some embodiments, the compound has

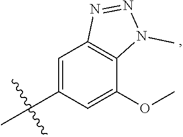 [SMILES: *c1cc(OC)c2c(c1)nnn2C]

wherein the [symbol] represents the connection to the rest of the structures of (I), (IA)-(ID), (IA-1), (IA-2), (IB-1), or (IB-2). In some embodiments, the compound has

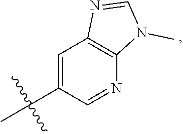

wherein the a [symbol] represents the connection to the rest of the structures of (I), (IA)-(ID), (IA-1), (IA-2), (IB-1), or (IB-2). In some embodiments, the compound has

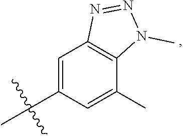 [SMILES: *c1cc(C)c2c(c1)nnn2C]

wherein the [symbol] represents the connection to the rest of the structures of (I), (IA)-(ID), (IA-1), (IA-2), (IB-1), or (IB-2). In some embodiments, the compound has

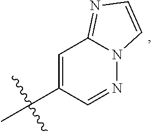 [SMILES: *c1cnn2ccnc2c1]

wherein the [symbol] represents the connection to the rest of the structures of (I), (IA)-(ID). (IA-1), (IA-2), (IB-1), or (IB-2). In some embodiments, the compound has

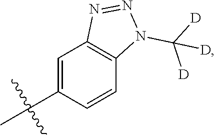

wherein the [symbol] represents the connection to the rest of the structures of (I), (IA)-(ID), (IA-1), (IA-2), (IB-1), or (IB-2). In some embodiments, the compound has

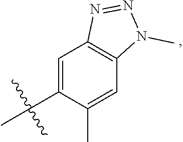 [SMILES: *c1cc2nnn(C)c2cc1C]

wherein the [symbol] represents the connection to the rest of the structures of (I), (IA)-(ID) (IA-1), (IA-2), (IB-1), or (IB-2). In some embodiments, the compound has

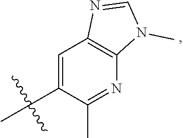 [SMILES: *c1cc2ncn(C)c2nc1C]

wherein the [symbol] represents the connection to the rest of the structures of (I), (IA)-(ID), (IA-I), (IA-2), (IB-1), or (IB-2). In some embodiments, the compound has

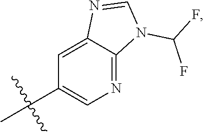 [SMILES: *c1cnc2c(c1)ncn2C(F)F]

wherein the [symbol] represents the connection to the rest of the structures of (I), (IA)-(ID), (IA-1), (IA-2), (IB-1), or (IB-2). In some embodiments, the compound has

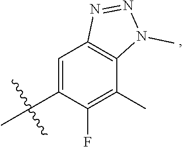 [SMILES: *c1cc2nnn(C)c2c(C)c1F]

wherein the [symbol] represents the connection to the rest of the structures of (I), (IA)-(ID), (IA-1), (IA-2), (IB-1), or (IB-2). In some embodiments, the compound has

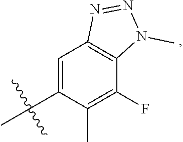 [SMILES: *c1cc2nnn(C)c2c(F)c1C]

wherein the [symbol] represents the connection to the rest of the structures of (I), (IA)-(ID), (IA-1), (IA-2), (IB-1), or (IB-2). In some embodiments, the compound has

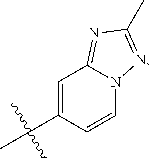 [SMILES: *c1ccn2nc(C)nc2c1]

wherein the [symbol] represents the connection to the rest of the structures of (I), (IA)-(ID), (IA-1), (IA-2), (IB-1), or (IB-2). In some embodiments, the compound has

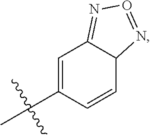

wherein the [symbol] represents the connection to the rest of the structures of (I), (IA)-(ID), (IA-1), (IA-2), (IB-1), or (IB-2). In some embodiments, the compound has

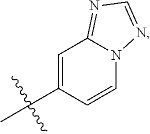 [SMILES: *c1ccn2ncnc2c1]

wherein the [symbol] represents the connection to the rest of the structures of (I), (IA)-(ID), (IA-1), (IA-2), (IB-1), or (IB-2). In some embodiments, the compound has

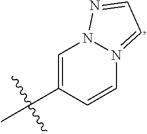

wherein the [symbol] represents the connection to the rest of the structures of (I), (IA)-(ID), (IA-1), (IA-2), (IB-1), or (IB-2).

In one embodiment, L is C₁-C₄ alkyl, C₃-C₈ cycloalkyl, C₃-C₈ heterocycloalkyl, a fused heterobicyclic, a bridged heterobicyclic, a hetero-spirocyclic, or a direct bond. In some embodiments, L is C₁-C₄ alkyl. In some embodiments, L is C₃-C₈ cycloalkyl. In some embodiments. L is C₃-C₈ heterocycloalkyl. In some embodiments. L is a fused heterobicyclic. In some embodiments, L is a bridged heterobicyclic. In some embodiments, L is a hetero-spirocyclic. In some embodiments, L is a direct bond.

In one embodiment, the C₃-C₈ heterocycloalkyl, the fused heterobicyclic, the bridged heterobicyclic, and the hetero-spirocyclic of L have 1-3 nitrogen atoms. In some embodiments, the C₃-C₈ heterocycloalkyl of L has 1-3 nitrogen atoms. In some embodiments, the fused heterobicyclic of L has 1-3 nitrogen atoms. In some embodiments, the bridged heterobicyclic of L has 1-3 nitrogen atoms. In some embodiments, the hetero-spirocyclic of L has 1-3 nitrogen atoms.

In one embodiment, the C₃-C₈ heterocycloalkyl, the fused heterobicyclic, the bridged heterobicyclic, and the hetero-spirocyclic of L have 1-2 nitrogen atoms. In some embodiments, the C₃-C₈ heterocycloalkyl of L has 1-2 nitrogen atoms. In some embodiments, the fused heterobicyclic of L has 1-2 nitrogen atoms. In some embodiments, the bridged heterobicyclic of L has 1-2 nitrogen atoms. In some embodiments, the hetero-spirocyclic of L has 1-2 nitrogen atoms.

In one embodiment, the C₃-C₈ heterocycloalkyl, the fused heterobicyclic, the bridged heterobicyclic, and the hetero-spirocyclic of L are unsaturated heterocycles. In some embodiments, the C₃-C₈ heterocycloalkyl of L is an unsaturated heterocycle. In some embodiments, the fused heterobicyclic of L is an unsaturated heterocycle. In some embodiments, the bridged heterobicyclic of L is an unsaturated heterocycle. In some embodiments, the hetero-spirocyclic of L is an unsaturated heterocycle.

In one embodiment, the C₃-C₈ heterocycloalkyl of L is azetidine, pyrrolidine, imidazolidine, pyrazolidine, piperidine, 1,2-diazinane, 1,3-diazinane, 1,4-diazinane, azapane, diazepane, or azocane. In some embodiments, the C₃-C₈ heterocycloalkyl of L is azetidine. In some embodiments, the C₃-C₈ heterocycloalkyl of L is pyrrolidine. In some embodiments, the C₃-C₈ heterocycloalkyl of L is imidazolidine. In some embodiments, the C₃-C₈ heterocycloalkyl of L is pyrazolidine. In some embodiments, the C₃-C₅ heterocycloalkyl of L is piperidine. In some embodiments, the C₃-C₈ heterocycloalkyl of L is 1,2-diazinane. In some embodiments, the C₃-C₈ heterocycloalkyl of L is 1,3-diazinane. In some embodiment, the C₃-C₈ heterocycloalkyl of L is 1,4-diazinane. In some embodiments, the C₃-C₈ heterocycloalkyl of L is azapane. In some embodiments, the C₃-C₈ heterocycloalkyl of L is diazepane. In some embodiments, the C₃-C₈ heterocycloalkyl of L is azocane.

In one embodiment, the fused heterobicyclic of L is 3-azabicyclo[3.1.0]heptane, 2,5-diazabicyclo[4.2.0]octane, octahydropyrrolo[3.4,c]pyrrole, octahydropyrrolo[3.4,b]pyrrole, octahydro-1-1-pyrrolo[3.4.c]pyridine, decahydro-2,6-naphthyridine, 2,5-diazabicyclo[4.1.0]heptane, 3,6-diazabicyclo[3.2.0]heptane, or 3,6-diazabicyclo[3.1.0]hexane. In some embodiments, the fused heterobicyclic of L is 3-azabicyclo[3.1.0]heptane. In some embodiments, the fused heterobicyclic of L is 2,5-diazabicyclo[4.2.0]octane. In some embodiments, the fused heterobicyclic of L is octahydropyrrolo[3.4.c]pyrrole. In some embodiments, the fused heterobicyclic of L is octahydro-1H-pyrrolo[3.4.c]pyridine. In some embodiments, the fused heterobicyclic of L is decahydro-2,6-naphthyridine. In some embodiments, the fused heterobicyclic of L is 2,5-diazabicyclo[4.1.0]heptane. In some embodiments, the fused heterobicyclic of L is 3,6-diazabicyclo[3.2.0]heptane. In some embodiments, the fused heterobicyclic of L is 3,6-diazabicyclo[3.1.0]hexane. In some embodiments, the fused heterobicyclic of L is octahydropyrrolo[3.4.b]pyrrole.

In one embodiment, the bridged heterobicyclic of L is 3,8-diazabicyclo[3.2.1]octane, 2,5-diazabicyclo[2.2.2]octane, 8-azabicyclo[3.2.1]octane, 3-azabicyclo[3.2.1]octane, 3,6-diazabicyclo[3.2.1]octane, 9-azabicyclo[3.3.11]nonane, 3-oxa-9-azabicyclo[3.3.1_]nonane, 3-oxa-9-azabicyclo[3.3.1]non-6-ene, 9-azabicyclo[3.3.1]non-2-ene, 8-azabicyclo[3.2.1]oct-2-ene, or 3,6-diazabicyclo[3.1.1]heptane. In some embodiments, the bridged heterobicyclic of L is 3,8-diazabicyclo[3.2.1]octane. In some embodiments, the bridged heterobicyclic of L is 2,5-diazabicyclo[2.2.2]octane. In some embodiments, the bridged heterobicyclic of L is 8-azabicyclo[3.2.1]octane. In some embodiments, the bridged heterobicyclic of L is 3-azabicyclo[3.2.1]octane. In some embodiments, the bridged heterobicyclic of L is 3,6-diazabicyclo[3.2.1]octane. In some embodiments, the bridged heterobicyclic of L is 9-azabicyclo[3.3.1]nonane. In some embodiments, the bridged heterobicyclic of L is 3-oxa-9-azabicyclo[3.3.1]nonane. In some embodiments, the bridged heterobicyclic of L is 3-oxa-9-azabicyclo[3.3.1]non-6-ene. In some embodiments, the bridged heterobicyclic of L is 9-azabicyclo[3.3.1]non-2-ene. In some embodiments, the bridged heterobicyclic of L is 8-azabicyclo[3.2.1]oct-2-ene. In some embodiments, the bridged heterobicyclic of L is 3,6-diazabicyclo[3.1.1]heptane.

In one embodiment, the hetero-spirocyclic of L is 2,6-diazaspiro[3.3]heptane, 1,6-diazaspiro[3.3]heptane, 2-azaspiro[3.3]heptane, 1,6-diazaspiro[3.4]octane, 2,6-diazaspiro[3.4]octane, 2,7-diazaspiro[3.5]nonane, 2,8-diazaspiro[4.5]decane, 3,9-diazaspiro[5.5]undecane, 4,8-diazaspiro[2.5]octane, 5,9-diazaspiro[3.5]nonane, 6,10-diazaspiro[4.5]decane, or 1,5-diazaspiro[5.5]undecane. In some embodiments, the hetero-spirocyclic of L is 2,6-diazaspiro[3.3]heptane. In some embodiments, the hetero-spirocyclic of L is 1,6-diazaspiro[3.3]heptane. In some embodiments, the hetero-spirocyclic of L is 2-azaspiro[3.3]heptane. In some embodiments, the hetero-spirocyclic of L is 1,6-diazaspiro[3.4]octane. In some embodiments, the hetero-spirocyclic of L is 2,6-diazaspiro[3.4]octane. In some embodiments, the hetero-spirocyclic of L is 2,7-diazaspiro[3.5]nonane. In some embodiments, the hetero-spirocyclic of L is 2,8-diazaspiro[4.5]decane. In some embodiments, the hetero-spirocyclic of L is 3,9-diazaspiro[5.5]undecane. In some embodiments, the hetero-spirocyclic of L is 4,8-diazaspiro[2.5]octane. In some embodiments, the hetero-spirocyclic of L is 5,9-diazaspiro[3.5]nonane. In some embodiments, the hetero-spirocyclic of L is 6,10-diazaspiro[4.5]decane. In some embodiments, the hetero-spirocyclic of L is 1,5-diazaspiro[5.5]undecane.

In one embodiment, the C₃-C₈ heterocycloalkyl, the fused heterobicyclic, the bridged heterobicyclic, and the hetero-spirocyclic of L are further substituted with deuterium, halo, C₁-C₆ alkyl, or C₁-C₆ heteroalkyl. In some embodiments, the C₃-C₈ heterocycloalkyl, the fused heterobicyclic, the bridged heterobicyclic, and the hetero-spirocyclic of L are further substituted with deuterium, halo, C₁-C₃ alkyl, or C₁-C₃ heteroalkyl. In some embodiments, the C₃-C₈ heterocycloalkyl, the fused heterobicyclic, the bridged heterobicyclic, and the hetero-spirocyclic of L are further substituted with -D, —F, —CH₃, —CF₃, —CHF₂, —CH₂F, —CH₂CHF₂, —C₂CH₃, —CH(CH₃)₂, —CH₂OH, —CH₂OCH₃,

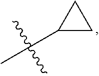 [SMILES: *C1CC1]

### or —CH₂CCN.

In one embodiment, the C₃-C₅ heterocycloalkyl is further substituted with -D, —F, —CH₃, —CF₃, —CHF₂, —CH₂F, —CH₂CHF₂, —CH₂CH₃, —CH(CH₃)₂, —CH₂OH, —CH₂OCH₃,

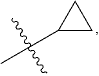 [SMILES: *C1CC1]

or —CH₂CCN. In some embodiments, the C₃-C₈ heterocycloalkyl is further substituted with -D. In some embodiments, the C₃-C₈ heterocycloalkyl is further substituted with —F. In some embodiments, the C₃-C₈ heterocycloalkyl is further substituted with —CH₃. In some embodiments, the C₃-C₈ heterocycloalkyl is further substituted with —CF₃. In some embodiments, the C₃-C₈ heterocycloalkyl is further substituted with —CHF₂, In some embodiments, the C₃-C₈ heterocycloalkyl is further substituted with —CH₂F. In some embodiments, the C₃-C₈ heterocycloalkyl is further substituted with —CH₂CHF₂. In some embodiments, the C₃-C₈ heterocycloalkyl is further substituted with —CH₂CH₃. In some embodiments, the C₃-C₈ heterocycloalkyl is further substituted with —CH(CH₃)₂. In some embodiments, the C₃-C₈ heterocycloalkyl is further substituted with —CH₂OH. In some embodiments, the C₃-C₈ heterocycloalkyl is further substituted with —CH₂OCH₃. In some embodiments, the C₃-C₈ heterocycloalkyl is further substituted with

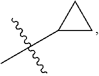 [SMILES: *C1CC1]

In some embodiments, the C₃-C₈ heterocycloalkyl is further substituted with —CH₂CCN.

In one embodiment, the fused heterobicyclic is further substituted with -D, —F, —CH₃, —CF₃, —CHF₂, —CH₂F, —CH₂CHF₂, —CH₂CH₃, —CH(CH₃)₂, —CH₂OH, —CH₂OCH₃,

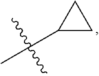 [SMILES: *C1CC1]

or —CH₂CCN. In some embodiments, the fused heterobicyclic is further substituted with -D, In some embodiments, the fused heterobicyclic is further substituted with —F. In some embodiments, the fused heterobicyclic is further substituted with —CH₃. In some embodiments, the fused heterobicyclic is further substituted with —CF₃. In some embodiments, the fused heterobicyclic is further substituted with —CHF₂. In some embodiments, the fused heterobicyclic is further substituted with —CH₂F, In some embodiments, the fused heterobicyclic is further substituted with —CH₂CHF₂. In some embodiments, the fused heterobicyclic is further substituted with —CH₂CH₃, In some embodiments, the fused heterobicyclic is further substituted with —CH(CH₃)₂. In some embodiments, the fused heterobicyclic is further substituted with —CH₂OH. In some embodiments, the fused heterobicyclic is further substituted with —CH₂OCH₃. In some embodiments, the fused heterobicyclic is further substituted with

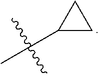 [SMILES: *C1CC1]

In some embodiments, the fused heterobicyclic is further substituted with —CH₂CCN.

In one embodiment, the bridged heterobicyclic is further substituted with -D, —F, —CH₃, —CF₃, —CHF₂, —CH₂F, —CH₂CHF₂, —CH₂CH₃, —CH(CH₃)₂, —CH₂OH, —CH₂OCH₃,

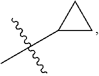 [SMILES: *C1CC1]

or —CH₂CCN. In some embodiments, the bridged heterobicyclic is further substituted with -D. In some embodiments, the bridged heterobicyclic is further substituted with —F. In some embodiments, the bridged heterobicyclic is further substituted with —CH₃. In some embodiments, the bridged heterobicyclic is further substituted with —CF₃. In some embodiments, the bridged heterobicyclic is further substituted with —CHF₂. In some embodiments, the bridged heterobicyclic is further substituted with —CH₂F. In some embodiments, the bridged heterobicyclic is further substituted with —CH₂CHF₂, In some embodiments, the bridged heterobicyclic is further substituted with —CH₂CH₃. In some embodiments, the bridged heterobicyclic is further substituted with —CH(CH₃)₂. In some embodiments, the bridged heterobicyclic is further substituted with CH₂OH. In some embodiments, the bridged heterobicyclic is further substituted with —CH₂OCH₃. In some embodiments, the bridged heterobicyclic is further substituted with

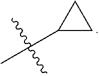 [SMILES: *C1CC1]

In some embodiments, the bridged heterobicyclic is further substituted with —CH₂CCN.

In one embodiment, the hetero-spirocyclic is further substituted with -D, —F, —CH₃, —CF₃, —CHF₂, —CH₂F, —CH₂CHF₂, —CH₂CH₃, —CH(CH₃)₂, —CH₂OH, —CH₂OCH₃,

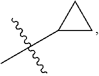 [SMILES: *C1CC1]

or —CH₂CCN. In some embodiments, the hetero-spirocyclic is further substituted with -D. In some embodiments, the hetero-spirocyclic is further substituted with —F. In some embodiments, the hetero-spirocyclic is further substituted with —CH₃. In some embodiments, the hetero-spirocyclic is further substituted with —CF₃, In some embodiments, the hetero-spirocyclic is further substituted with —CHF₂. In some embodiments, the hetero-spirocyclic is further substituted with —CH₂F. In some embodiments, the hetero-spirocyclic is further substituted with —CH₂CHF₂. In some embodiments, the hetero-spirocyclic is further substituted with —CH₂CH₃. In some embodiments, the hetero-spirocyclic is further substituted with —CH(CH₃)₂. In some embodiments, the hetero-spirocyclic is further substituted with —CH₂OH. In some embodiments, the hetero-spirocyclic is further substituted with —CH₂OCH₃. In some embodiments, the hetero-spirocyclic is further substituted with

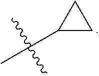 [SMILES: *C1CC1]

In some embodiments, the hetero-spirocyclic is further substituted with —CH₂CCN.

In one embodiment, the C₃-C₈ heterocycloalkyl, the fused heterobicyclic, the bridged heterobicyclic, and the hetero-spirocyclic of L are mono-, di-, tri-, tetra-, penta-, or octa-substituted. In some embodiments, the C₃-C₈ heterocycloalkyl of L is mono-, di-, tri-, tetra-, penta-, or octa-substituted. In some embodiments, the fused heterobicyclic of L is mono-, di-, tri-, tetra-, penta-, or octa-substituted. In some embodiments, the bridged heterobicyclic of L is mono-, di-, tri-, tetra-, penta-, or octa-substituted. In some embodiments, the hetero-spirocyclic of L is mono-, di-, tri-, tetra-, penta-, or octa-substituted.

In one embodiment, L is C₂-C₄ alkyl or C₄-C₅ cycloalkyl. In some embodiments, L is C₂-C₄ alkyl. In some embodiments, L is C₄-C₅ cycloalkyl, In some embodiments, C₁-C₄ alkyl of L is —(CH₂)₂— or —CH₂C(CH₃)₂. In some embodiments, C₁-C₄ alkyl of L is —(CH₂)₂—. In some embodiments, C₁-C₄ alkyl of L is —CH₂C(CH₃)₂.

In one embodiment, C₃-C₈ cycloalkyl of L is

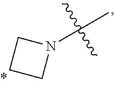 [SMILES: *N1CCC1]

wherein * indicates a location of a bond to Z².
In one embodiment, L has one of the following structures:

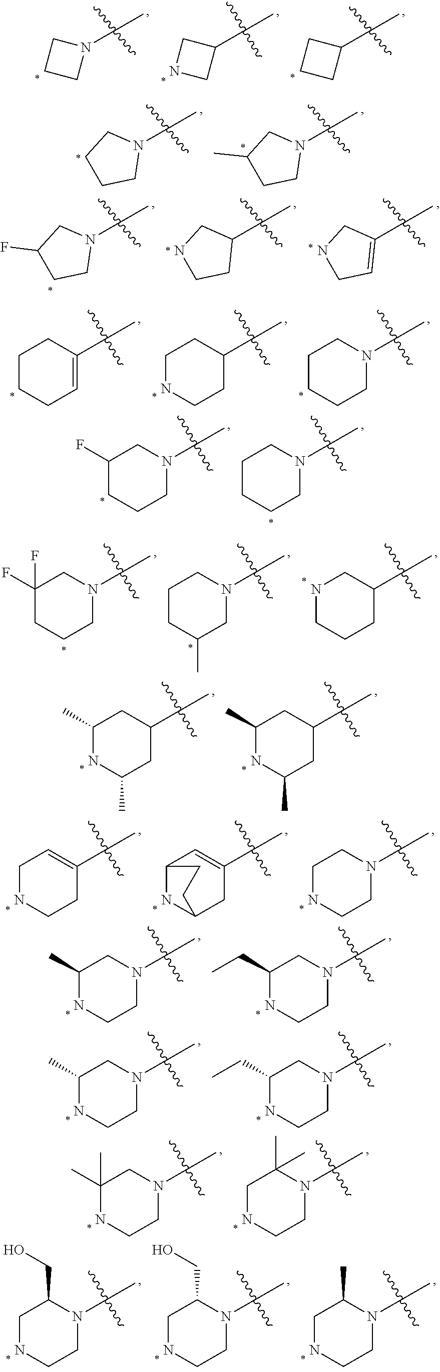

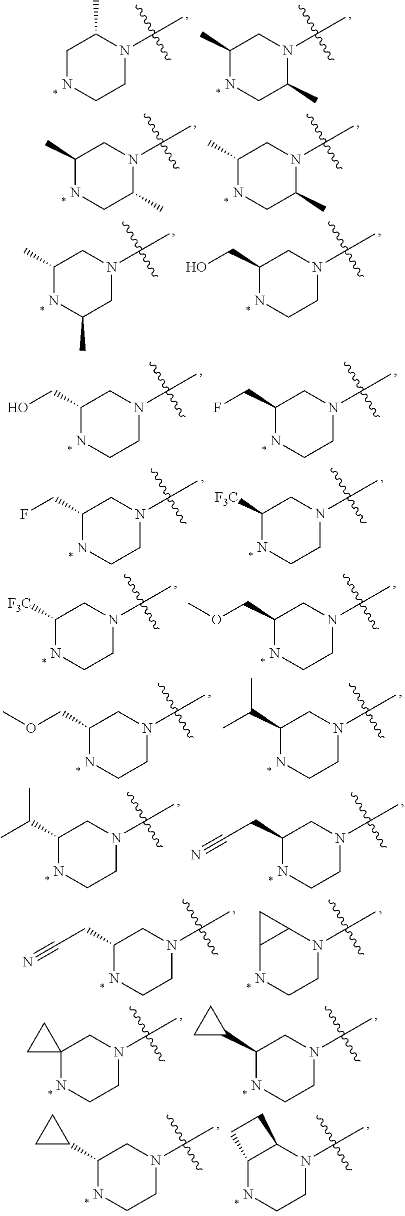

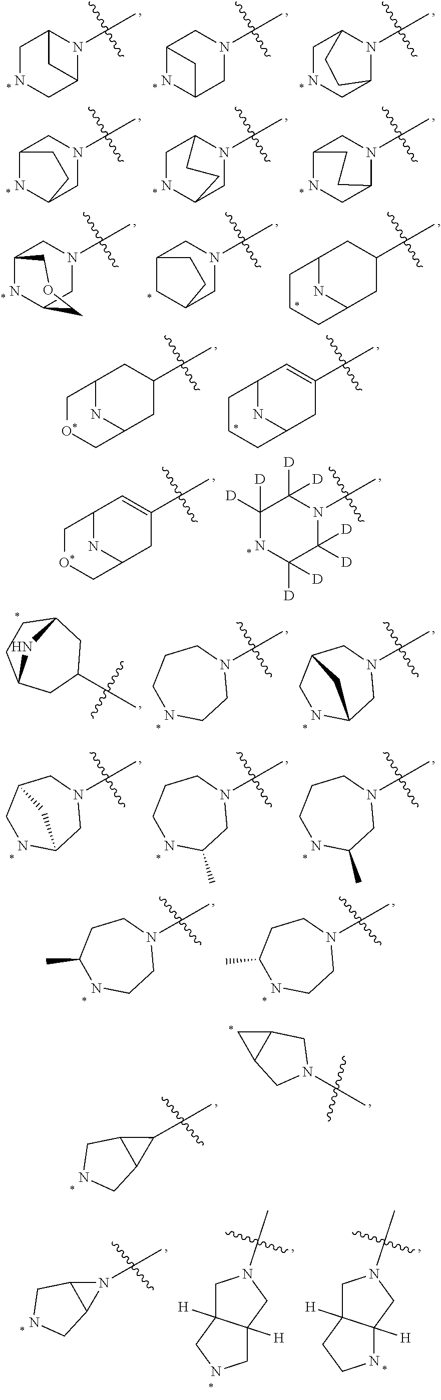

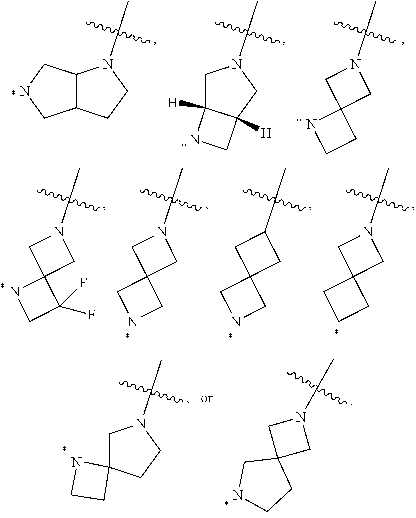

wherein * indicates a location of a bond to Z². In some embodiments, L is

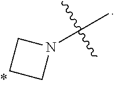 [SMILES: *N1CCC1]

In some embodiments, L is

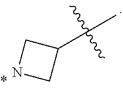 [SMILES: *C1CNC1]

In some embodiments, L is

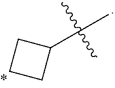 [SMILES: *C1CCC1]

In some embodiments, L is

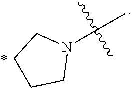 [SMILES: *N1CCCC1]

In some embodiments, L is

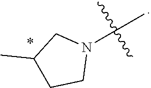 [SMILES: *N1CCC(C)C1]

In some embodiments, L is

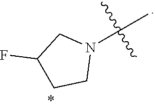 [SMILES: *N1CCC(F)C1]

In some embodiments, L is

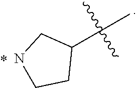 [SMILES: *C1CCNC1]

In some embodiments, L is

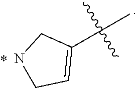 [SMILES: *C1=CCNC1]

In some embodiments, L is

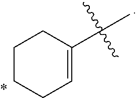 [SMILES: *C1=CCCCC1]

In some embodiments, L is

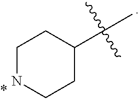 [SMILES: *C1CCNCC1]

In some embodiments, L is

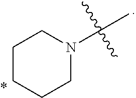 [SMILES: *N1CCCCC1]

In some embodiments, L is

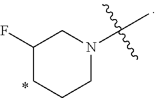 [SMILES: *N1CCCC(F)C1]

In some embodiments, L is

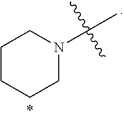 [SMILES: *N1CCCCC1]

In some embodiments, L is

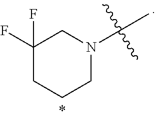 [SMILES: *N1CCCC(F)(F)C1]

In some embodiments, L is

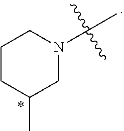 [SMILES: *N1CCCC(C)C1]

In some embodiments, L is

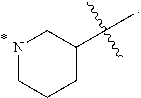 [SMILES: *C1CCCNC1]

In some embodiments, L is

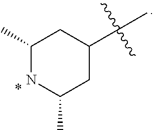 [SMILES: *C1C[C@@H](C)N[C@@H](C)C1]

In some embodiments, L is

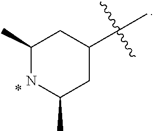 [SMILES: *C1C[C@@H](C)N[C@@H](C)C1]

In some embodiments, L is

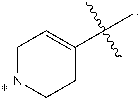 [SMILES: *C1=CCNCC1]

In some embodiments, L is

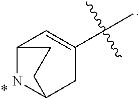 [SMILES: *C1=CC2CCC(C1)N2]

In some embodiments, L is

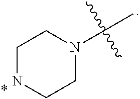 [SMILES: *N1CCNCC1]

In some embodiments, L is

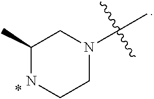 [SMILES: *N1CCN[C@@H](C)C1]

In some embodiments, L is

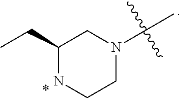 [SMILES: *N1CCN[C@@H](CC)C1]

In embodiments, L is

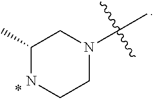 [SMILES: *N1CCN[C@H](C)C1]

In some embodiments, L is

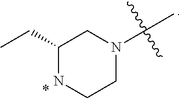 [SMILES: *N1CCN[C@H](CC)C1]

In some embodiments, L is

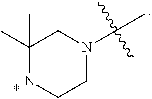 [SMILES: *N1CCNC(C)(C)C1]

In some embodiments, L is

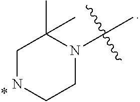 [SMILES: *N1CCNCC1(C)C]

In some embodiments, L is

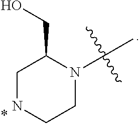 [SMILES: *N1CCNC[C@H]1CO]

In some embodiments, L is

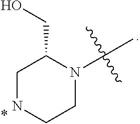 [SMILES: *N1CCNC[C@@H]1CO]

In some embodiments, L is

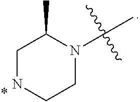 [SMILES: *N1CCNC[C@H]1C]

In some embodiments, L is

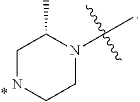 [SMILES: *N1CCNC[C@@H]1C]

In some embodiments, L is

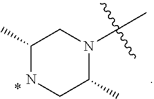 [SMILES: *N1C[C@@H](C)NC[C@H]1C]

In some embodiments, L is

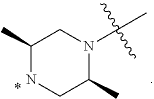 [SMILES: *N1C[C@H](C)NC[C@@H]1C]

In some embodiments, L is

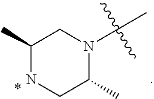 [SMILES: *N1C[C@H](C)NC[C@H]1C]

In some embodiments, L is

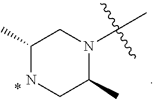 [SMILES: *N1C[C@@H](C)NC[C@@H]1C]

In some embodiments, L is

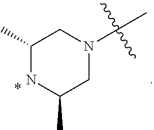 [SMILES: *N1C[C@@H](C)N[C@H](C)C1]

In some embodiments, L is

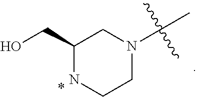 [SMILES: *N1CCN[C@@H](CO)C1]

In some embodiments, L is

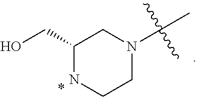 [SMILES: *N1CCN[C@H](CO)C1]

In some embodiments, L is

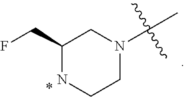 [SMILES: *N1CCN[C@@H](CF)C1]

In some embodiments, L is

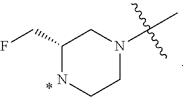 [SMILES: *N1CCN[C@H](CF)C1]

In some embodiments, L is

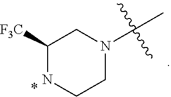 [SMILES: *N1CCN[C@@H](C(F)(F)F)C1]

In some embodiments, L is

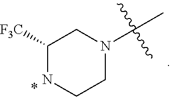 [SMILES: *N1CCN[C@H](C(F)(F)F)C1]

In some embodiments, L is

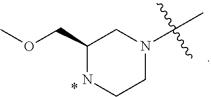 [SMILES: *N1CCN[C@@H](COC)C1]

In some embodiments, L is

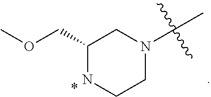 [SMILES: *N1CCN[C@H](COC)C1]

In some embodiments, L is

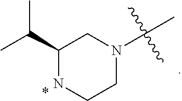 [SMILES: *N1CCN[C@@H](C(C)C)C1]

In some embodiments, L is

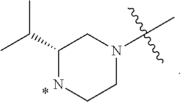 [SMILES: *N1CCN[C@H](C(C)C)C1]

In some embodiments, L is

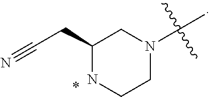

In some embodiments, L is

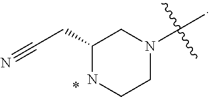

In some embodiments, L is

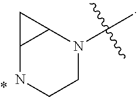

In some embodiments, L is

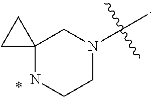

In some embodiments, L is

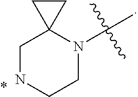

In some embodiments, L is

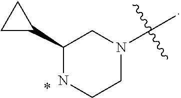

In some embodiments, L is

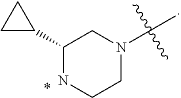

In some embodiments, L is

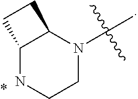

some embodiments, L is

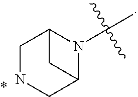

In some embodiments, L is

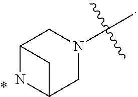

In some embodiments, L is

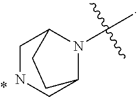

In some embodiments, L is

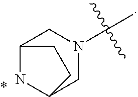

In some embodiments, L is

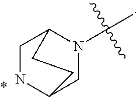

In some embodiments, L is

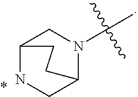

In some embodiments, L is

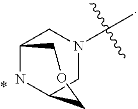

In some embodiments, L is

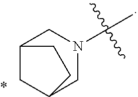

In some embodiments, L is

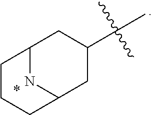 [SMILES: *C1CC2CCCC(C1)N2]

In some embodiments, L is

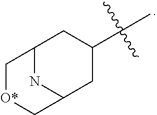

In some embodiments, L is

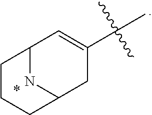 [SMILES: *C1=CC2CCCC(C1)N2]

In some embodiments, L is

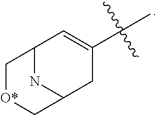

In some embodiments, L is

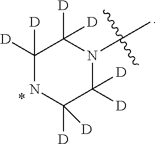 [SMILES: *N1C([2H])([2H])C([2H])([2H])NC([2H])([2H])C1([2H])[2H]]

In some embodiments, L is

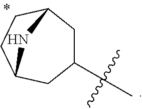 [SMILES: *C1C[C@H]2CC[C@@H](C1)N2 ; "."]

In some embodiments, L is

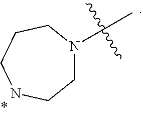 [SMILES: "." ; *N1CCCNCC1]

In some embodiments, L is

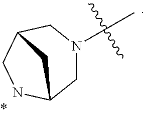 [SMILES: "." ; *N1C[C@H]2CN[C@H](C2)C1]

In some embodiments, L is

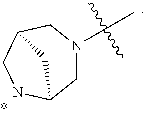 [SMILES: "." ; *N1C[C@@H]2CN[C@@H](C2)C1]

In some embodiments, L is

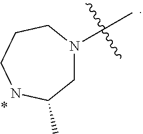 [SMILES: "." ; *N1CCCN[C@@H](C)C1]

In some embodiments, L is

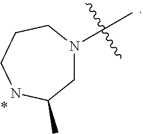 [SMILES: "." ; *N1CCCN[C@H](C)C1]

In some embodiments, L is

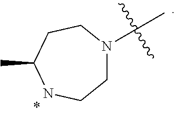 [SMILES: "." ; *N1CCN[C@@H](C)CC1]

In some embodiments, L is

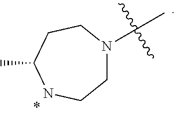 [SMILES: "." ; *N1CCN[C@H](C)CC1]

In some embodiments, L is

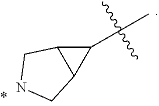 [SMILES: "." ; *C1C2CNCC12]

In some embodiments, L is

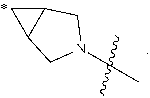 [SMILES: *N1CC2CC2C1 ; "."]

In some embodiments, L is

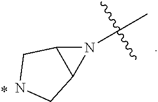 [SMILES: *N1C2CNCC21]

In some embodiments, L is

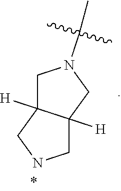 [SMILES: *N1CC2CNCC2C1]

In some embodiments, L is

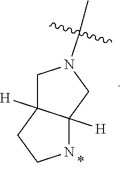 [SMILES: *N1CC2CCNC2C1]

In some embodiments, L is

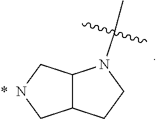

In some embodiments, L is

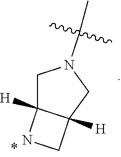 [SMILES: *N1C[C@@H]2CN[C@@H]2C1]

In some embodiments, L is

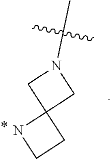 [SMILES: *N1CC2(CCN2)C1]

In some embodiments, L is

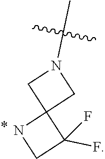 [SMILES: *N1CC2(C1)NCC2(F)F]

In some embodiments, L is

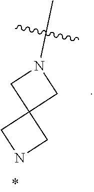 [SMILES: *N1CC2(CNC2)C1]

In some embodiments, L is

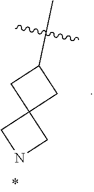 [SMILES: *C1CC2(CNC2)C1]

In some embodiments L is

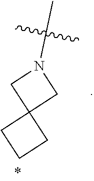 [SMILES: *N1CC2(CCC2)C1]

In some embodiments, L is

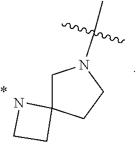 [SMILES: *N1CCC2(CCN2)C1]

In some embodiments, L is

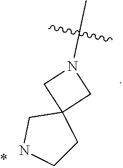 [SMILES: *N1CC2(CCNC2)C1]

In one embodiment, E is α,β-unsaturated carbonyl or C₂-C₄ alkyne conjugated carbonyl. In some embodiments, E is α,β-unsaturated carbonyl. In some embodiments, E is C₂-C₄ alkyne conjugated carbonyl. In some embodiments, α,β-unsaturated carbonyl or C₂-C₄ alkyne conjugated carbonyl of E is further substituted with halo, C₁-C₃ alkyl, C₁-C₃ alkylhalo, C₁-C₆ heteroalkyl, —(CH₂)ₙC₃-C₇ heterocycloalkyl, or combination thereof, wherein n is an integer between 1-3.

In one embodiment, α,β-unsaturated carbonyl of E has one of the following structures:

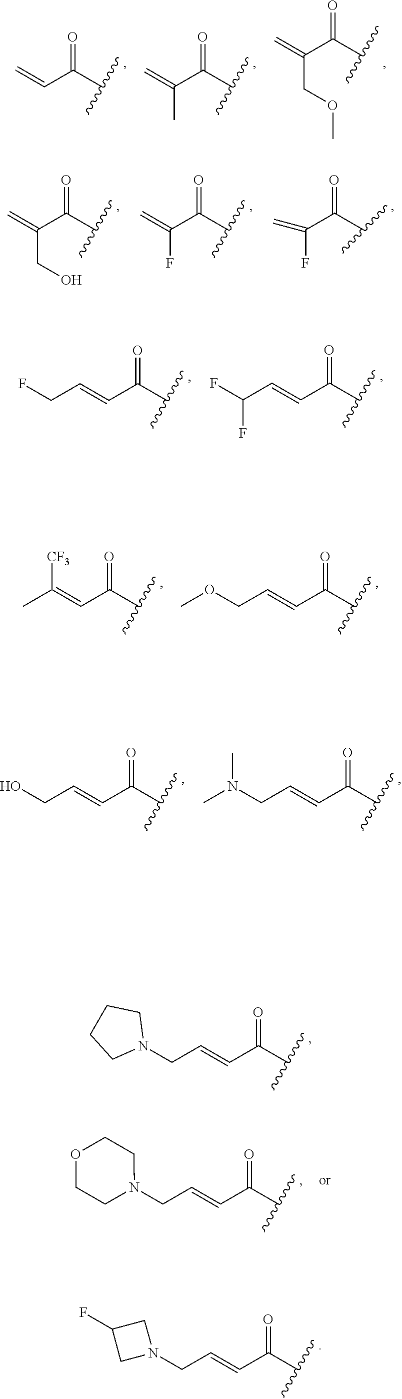

or In some embodiments, α,β-unsaturated carbonyl of E is

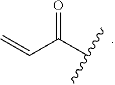 [SMILES: *C(=O)C=C ; "."]

In some embodiments, α, β-unsaturated carbonyl of E is

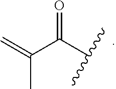 [SMILES: *C(=O)C(=C)C ; "."]

In some embodiments, α,β-unsaturated carbonyl of E is

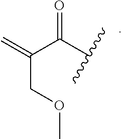 [SMILES: *C(=O)C(=C)COC]

In some embodiments, α,β-unsaturated carbonyl of E is

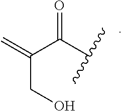 [SMILES: *C(=O)C(=C)CO]

In some embodiments, α,β-unsaturated carbonyl of E is

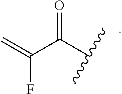 [SMILES: *C(=O)C(=C)F]

In some embodiments, α,β-unsaturated carbonyl of E is

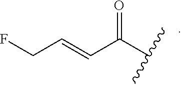 [SMILES: *C(=O)/C=C/CF]

In some embodiments, α,β-unsaturated carbonyl of E is

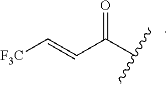 [SMILES: *C(=O)/C=C/C(F)(F)F]

In some embodiments, α,β-unsaturated carbonyl of E is

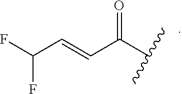 [SMILES: *C(=O)/C=C/C(F)F]

In some embodiments, α,β-unsaturated carbonyl of E is

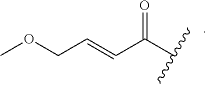 [SMILES: *C(=O)/C=C/COC]

In some embodiments, α,β-unsaturated carbonyl of E is

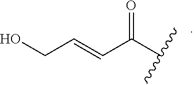 [SMILES: *C(=O)/C=C/CO]

In some embodiments, α,β-unsaturated carbonyl of E is

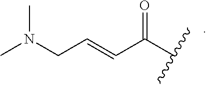 [SMILES: *C(=O)/C=C/CN(C)C]

In some embodiments, α,β-unsaturated carbonyl of E is

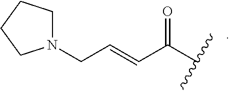 [SMILES: *C(=O)/C=C/CN1CCCC1]

In some embodiments, α,β-unsaturated carbonyl of E is

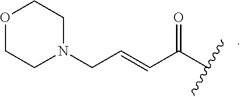 [SMILES: *C(=O)/C=C/CN1CCOCC1]

In some embodiments, α,β-unsaturated carbonyl of E is

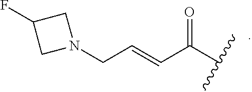 [SMILES: *C(=O)/C=C/CN1CC(F)C1]

In one embodiment, the C₂-C₄ alkyne conjugated carbonyl is C₂ alkyne conjugated carbonyl or C₃ alkyne conjugated carbonyl. In some embodiments, the C₂-C₄ alkyne conjugated carbonyl is C₂ alkyne conjugated carbonyl. In some embodiments, the C₂-C₄ alkyne conjugated carbonyl is C₃ alkyne conjugated carbonyl. In some certain embodiments, the C₂-C₄ alkyne conjugated carbonyl has the following structure:

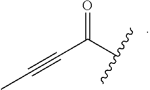 [SMILES: *C(=O)C#CC]

In some embodiments, the C₂-C₄ alkyne conjugated carbonyl is

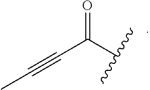 [SMILES: *C(=O)C#CC]

In one embodiment, E has one of the following structures:

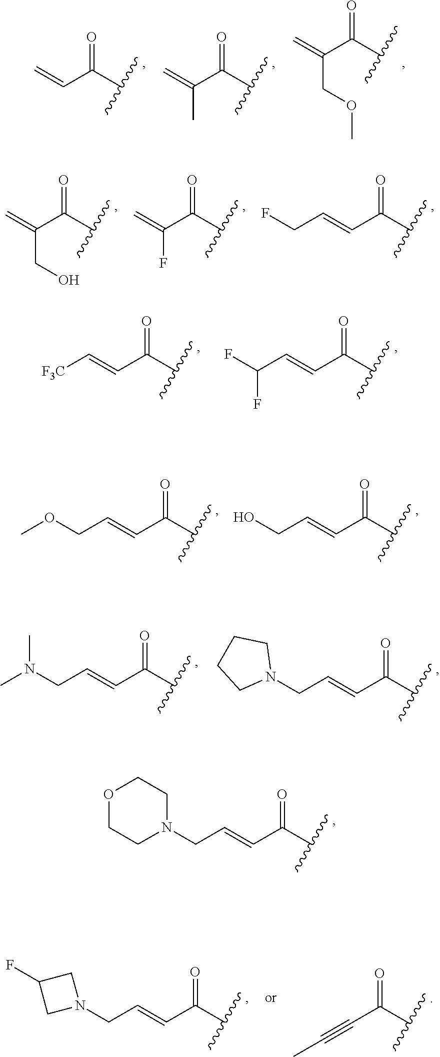

In some embodiments, E is

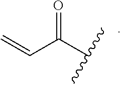 [SMILES: *C(=O)C=C]

In some embodiments, E is

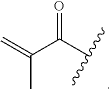 [SMILES: *C(=O)C(=C)C ; "."]

In some embodiments, E is

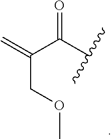 [SMILES: *C(=O)C(=C)COC]

In some embodiments, E is

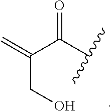 [SMILES: *C(=O)C(=C)CO]

In some embodiments, E is

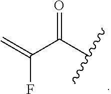 [SMILES: *C(=O)C(=C)F]

In some embodiments, E is

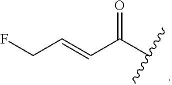 [SMILES: *C(=O)/C=C/CF]

In some embodiments, E is

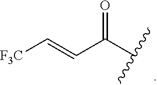 [SMILES: *C(=O)/C=C/C(F)(F)F]

In some embodiments, E is

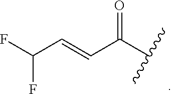 [SMILES: *C(=O)/C=C/C(F)F]

In some embodiments, E is

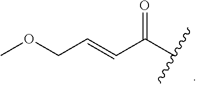 [SMILES: *C(=O)/C=C/COC]

In some embodiments, E is

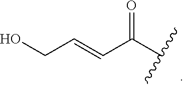 [SMILES: *C(=O)/C=C/CO]

In some embodiments, E is

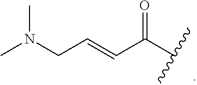 [SMILES: *C(=O)/C=C/CN(C)C]

In some embodiments, E is

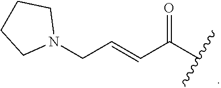 [SMILES: *C(=O)/C=C/CN1CCCC1]

In some embodiments, E is

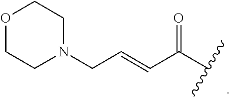 [SMILES: *C(=O)/C=C/CN1CCOCC1]

In some embodiments, E is

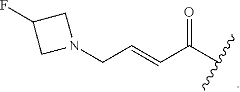 [SMILES: *C(=O)/C=C/CN1CC(F)C1]

In some embodiments, E is

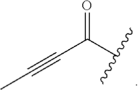 [SMILES: *C(=O)C#CC]

In one embodiment, the compound of formula of (I), (IA)-(ID), (IA-1), (IA-2), (IB-1), or (IB-2) has one of the following structures shown in Table 1 below.

TABLE 1
List of Compounds of (I), (IA)-(ID), (IA-1), (IA-2), (IB-1), or (IB-2)

| Cmpd | |
|---|---|
| ID | Structure |
| 1 | 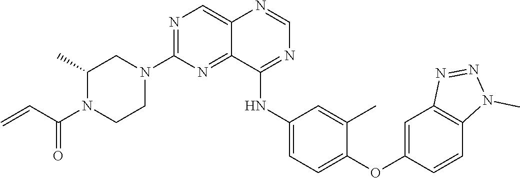 [SMILES: C=CC(=O)N1CCN(c2ncc3ncnc(Nc4ccc(Oc5ccc6c(c5)nnn6C)c(C)c4)c3n2)C[C@H]1C] |
| 2 | 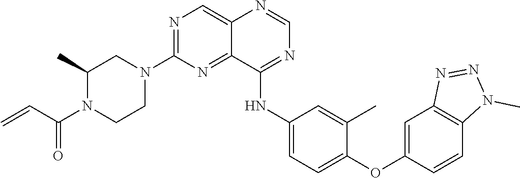 [SMILES: C=CC(=O)N1CCN(c2ncc3ncnc(Nc4ccc(Oc5ccc6c(c5)nnn6C)c(C)c4)c3n2)C[C@@H]1C] |
| 3 | 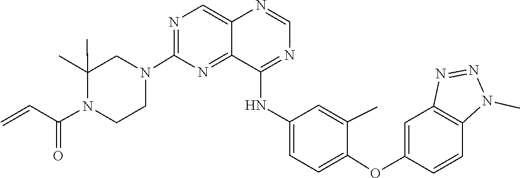 [SMILES: C=CC(=O)N1CCN(c2ncc3ncnc(Nc4ccc(Oc5ccc6c(c5)nnn6C)c(C)c4)c3n2)CC1(C)C] |
| 4 | 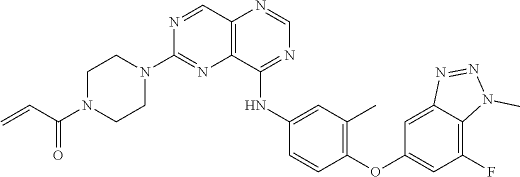 [SMILES: C=CC(=O)N1CCN(c2ncc3ncnc(Nc4ccc(Oc5cc(F)c6c(c5)nnn6C)c(C)c4)c3n2)CC1] |
| 5 | 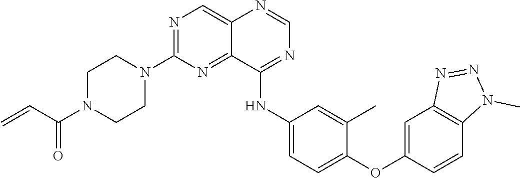 [SMILES: C=CC(=O)N1CCN(c2ncc3ncnc(Nc4ccc(Oc5ccc6c(c5)nnn6C)c(C)c4)c3n2)CC1] |
| 6 | 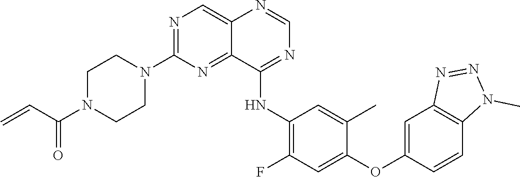 [SMILES: C=CC(=O)N1CCN(c2ncc3ncnc(Nc4cc(C)c(Oc5ccc6c(c5)nnn6C)cc4F)c3n2)CC1] |
| 7 | 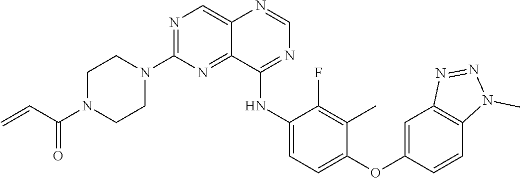 [SMILES: C=CC(=O)N1CCN(c2ncc3ncnc(Nc4ccc(Oc5ccc6c(c5)nnn6C)c(C)c4F)c3n2)CC1] |
| 8 | 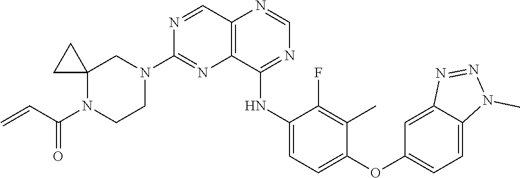 [SMILES: C=CC(=O)N1CCN(c2ncc3ncnc(Nc4ccc(Oc5ccc6c(c5)nnn6C)c(C)c4F)c3n2)CC12CC2] |
| 9 | 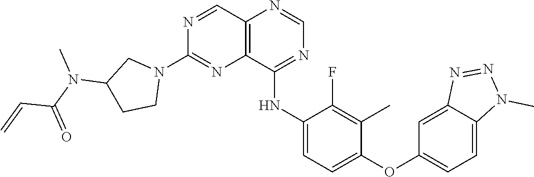 [SMILES: C=CC(=O)N(C)C1CCN(c2ncc3ncnc(Nc4ccc(Oc5ccc6c(c5)nnn6C)c(C)c4F)c3n2)C1] |
| 10 | 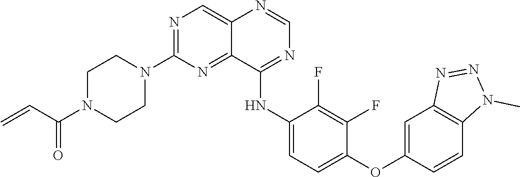 |
| 11 | 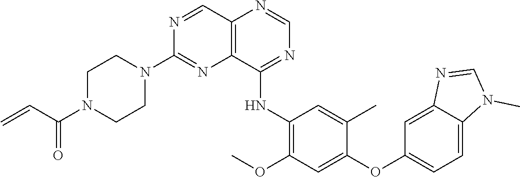 [SMILES: C=CC(=O)N1CCN(c2ncc3ncnc(Nc4cc(C)c(Oc5ccc6c(c5)ncn6C)cc4OC)c3n2)CC1] |
| 12 | 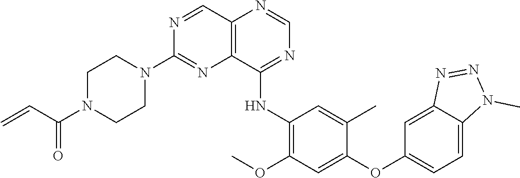 [SMILES: C=CC(=O)N1CCN(c2ncc3ncnc(Nc4cc(C)c(Oc5ccc6c(c5)nnn6C)cc4OC)c3n2)CC1] |
| 13 | 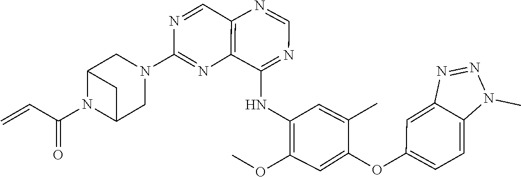 [SMILES: C=CC(=O)N1C2CC1CN(c1ncc3ncnc(Nc4cc(C)c(Oc5ccc6c(c5)nnn6C)cc4OC)c3n1)C2] |
| 14 | 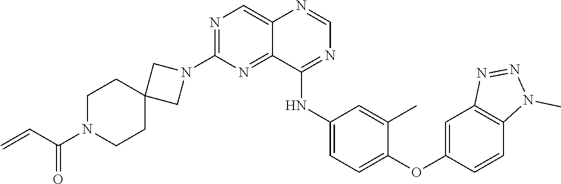 [SMILES: C=CC(=O)N1CCC2(CC1)CN(c1ncc3ncnc(Nc4ccc(Oc5ccc6c(c5)nnn6C)c(C)c4)c3n1)C2] |
| 15 | 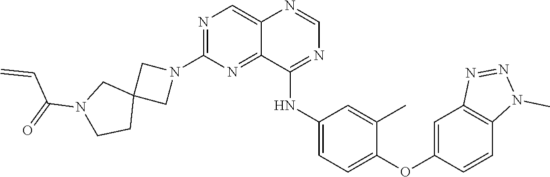 [SMILES: C=CC(=O)N1CCC2(C1)CN(c1ncc3ncnc(Nc4ccc(Oc5ccc6c(c5)nnn6C)c(C)c4)c3n1)C2] |
| 16 | 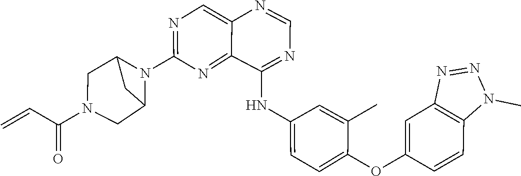 [SMILES: C=CC(=O)N1CC2CC(C1)N2c1ncc2ncnc(Nc3ccc(Oc4ccc5c(c4)nnn5C)c(C)c3)c2n1] |
| 17 | 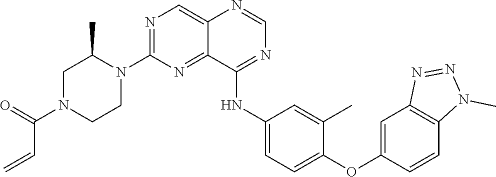 [SMILES: C=CC(=O)N1CCN(c2ncc3ncnc(Nc4ccc(Oc5ccc6c(c5)nnn6C)c(C)c4)c3n2)[C@H](C)C1] |
| 18 | 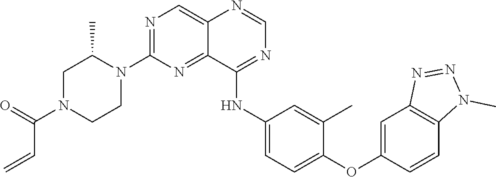 [SMILES: C=CC(=O)N1CCN(c2ncc3ncnc(Nc4ccc(Oc5ccc6c(c5)nnn6C)c(C)c4)c3n2)[C@@H](C)C1] |
| 19 | 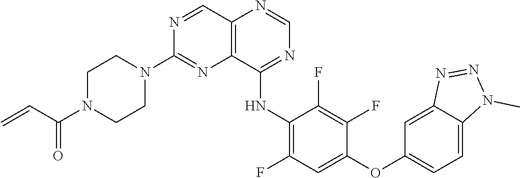 [SMILES: C=CC(=O)N1CCN(c2ncc3ncnc(Nc4c(F)cc(Oc5ccc6c(c5)nnn6C)c(F)c4F)c3n2)CC1] |
| 20 | 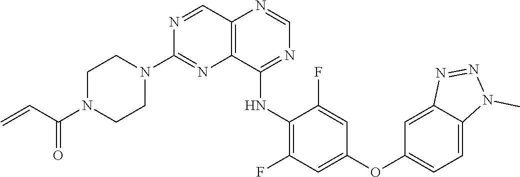 [SMILES: C=CC(=O)N1CCN(c2ncc3ncnc(Nc4c(F)cc(Oc5ccc6c(c5)nnn6C)cc4F)c3n2)CC1] |
| 21 | 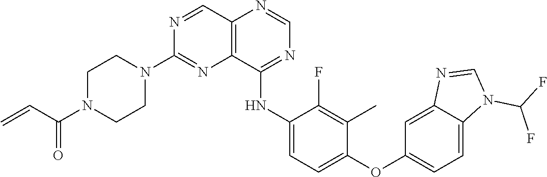 [SMILES: C=CC(=O)N1CCN(c2ncc3ncnc(Nc4ccc(Oc5ccc6c(c5)ncn6C(F)F)c(C)c4F)c3n2)CC1] |
| 22 | 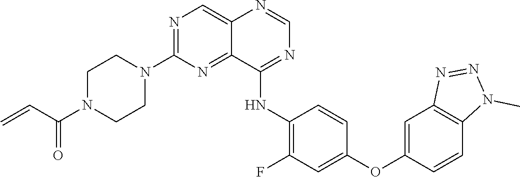 [SMILES: C=CC(=O)N1CCN(c2ncc3ncnc(Nc4ccc(Oc5ccc6c(c5)nnn6C)cc4F)c3n2)CC1] |
| 23 | 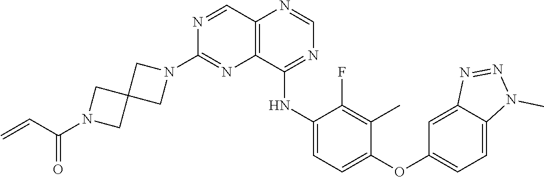 [SMILES: C=CC(=O)N1CC2(C1)CN(c1ncc3ncnc(Nc4ccc(Oc5ccc6c(c5)nnn6C)c(C)c4F)c3n1)C2] |
| 24 | 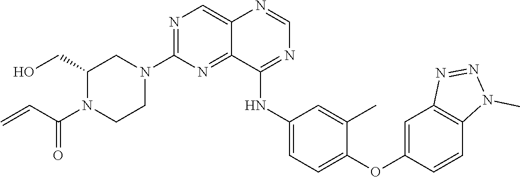 [SMILES: C=CC(=O)N1CCN(c2ncc3ncnc(Nc4ccc(Oc5ccc6c(c5)nnn6C)c(C)c4)c3n2)C[C@H]1CO] |
| 25 | 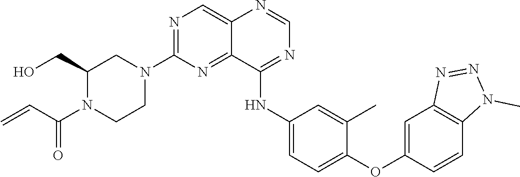 [SMILES: C=CC(=O)N1CCN(c2ncc3ncnc(Nc4ccc(Oc5ccc6c(c5)nnn6C)c(C)c4)c3n2)C[C@@H]1CO] |
| 26 | 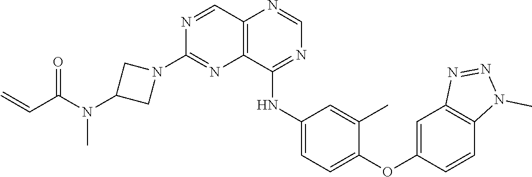 [SMILES: C=CC(=O)N(C)C1CN(c2ncc3ncnc(Nc4ccc(Oc5ccc6c(c5)nnn6C)c(C)c4)c3n2)C1] |
| 27 | 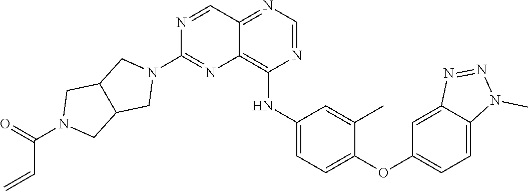 [SMILES: C=CC(=O)N1CC2CN(c3ncc4ncnc(Nc5ccc(Oc6ccc7c(c6)nnn7C)c(C)c5)c4n3)CC2C1] |
| 28 | 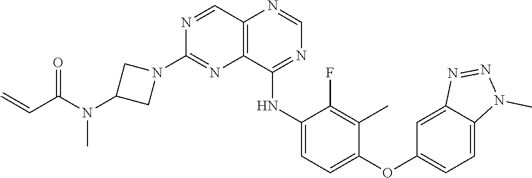 [SMILES: C=CC(=O)N(C)C1CN(c2ncc3ncnc(Nc4ccc(Oc5ccc6c(c5)nnn6C)c(C)c4F)c3n2)C1] |
| 29 | 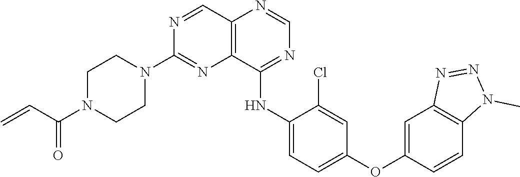 [SMILES: C=CC(=O)N1CCN(c2ncc3ncnc(Nc4ccc(Oc5ccc6c(c5)nnn6C)cc4Cl)c3n2)CC1] |
| 30 | 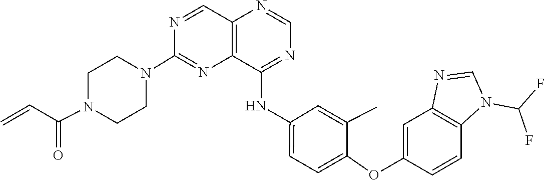 [SMILES: C=CC(=O)N1CCN(c2ncc3ncnc(Nc4ccc(Oc5ccc6c(c5)ncn6C(F)F)c(C)c4)c3n2)CC1] |
| 31 | 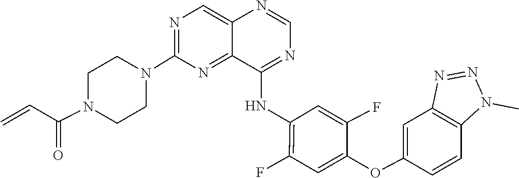 [SMILES: C=CC(=O)N1CCN(c2ncc3ncnc(Nc4cc(F)c(Oc5ccc6c(c5)nnn6C)cc4F)c3n2)CC1] |
| 32 | 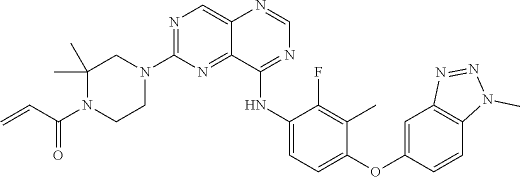 [SMILES: C=CC(=O)N1CCN(c2ncc3ncnc(Nc4ccc(Oc5ccc6c(c5)nnn6C)c(C)c4F)c3n2)CC1(C)C] |
| 33 | 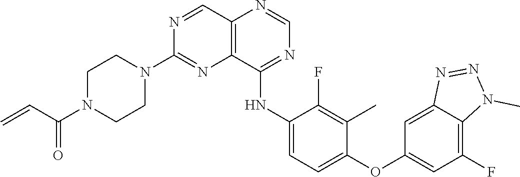 [SMILES: C=CC(=O)N1CCN(c2ncc3ncnc(Nc4ccc(Oc5cc(F)c6c(c5)nnn6C)c(C)c4F)c3n2)CC1] |
| 34 | 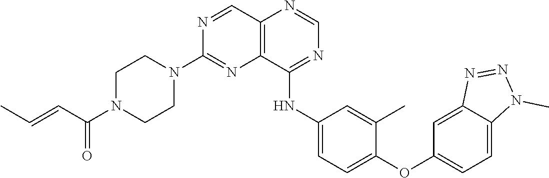 [SMILES: C/C=C/C(=O)N1CCN(c2ncc3ncnc(Nc4ccc(Oc5ccc6c(c5)nnn6C)c(C)c4)c3n2)CC1] |
| 35 | 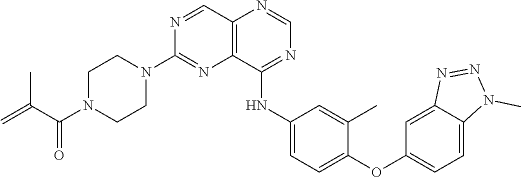 [SMILES: C=C(C)C(=O)N1CCN(c2ncc3ncnc(Nc4ccc(Oc5ccc6c(c5)nnn6C)c(C)c4)c3n2)CC1] |
| 36 | 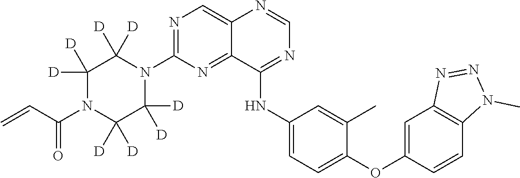 [SMILES: [2H]C1([2H])N(C(=O)C=C)C([2H])([2H])C([2H])([2H])N(c2ncc3ncnc(Nc4ccc(Oc5ccc6c(c5)nnn6C)c(C)c4)c3n2)C1([2H])[2H]] |
| 37 | 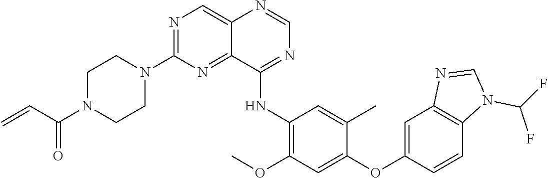 [SMILES: C=CC(=O)N1CCN(c2ncc3ncnc(Nc4cc(C)c(Oc5ccc6c(c5)ncn6C(F)F)cc4OC)c3n2)CC1] |
| 38 | 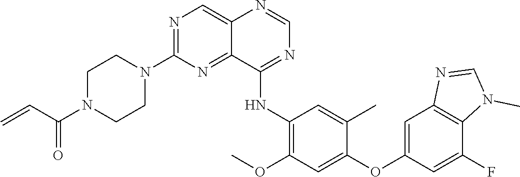 [SMILES: C=CC(=O)N1CCN(c2ncc3ncnc(Nc4cc(C)c(Oc5cc(F)c6c(c5)ncn6C)cc4OC)c3n2)CC1] |
| 39 | 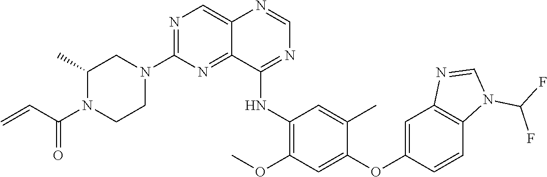 [SMILES: C=CC(=O)N1CCN(c2ncc3ncnc(Nc4cc(C)c(Oc5ccc6c(c5)ncn6C(F)F)cc4OC)c3n2)C[C@H]1C] |
| 40 | 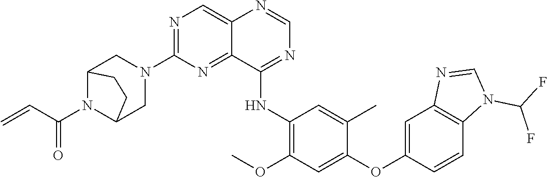 [SMILES: C=CC(=O)N1C2CCC1CN(c1ncc3ncnc(Nc4cc(C)c(Oc5ccc6c(c5)ncn6C(F)F)cc4OC)c3n1)C2] |
| 41 | 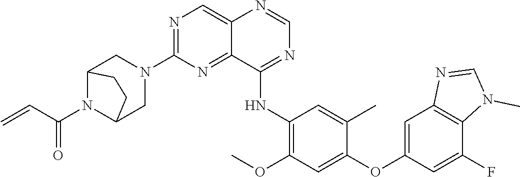 [SMILES: C=CC(=O)N1C2CCC1CN(c1ncc3ncnc(Nc4cc(C)c(Oc5cc(F)c6c(c5)ncn6C)cc4OC)c3n1)C2] |
| 42 | 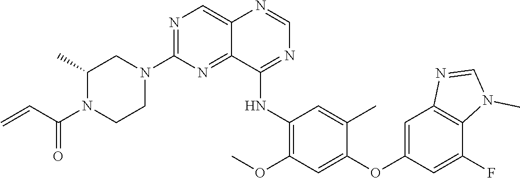 [SMILES: C=CC(=O)N1CCN(c2ncc3ncnc(Nc4cc(C)c(Oc5cc(F)c6c(c5)ncn6C)cc4OC)c3n2)C[C@H]1C] |
| 43 | 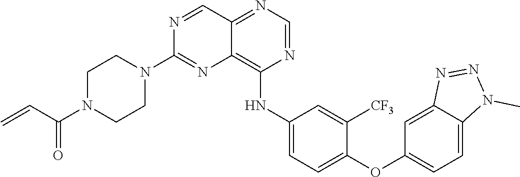 [SMILES: C=CC(=O)N1CCN(c2ncc3ncnc(Nc4ccc(Oc5ccc6c(c5)nnn6C)c(C(F)(F)F)c4)c3n2)CC1] |
| 44 | 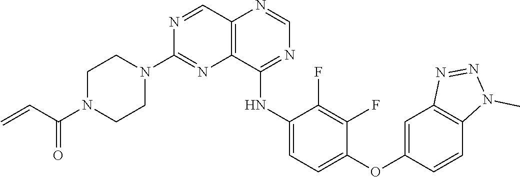 [SMILES: C=CC(=O)N1CCN(c2ncc3ncnc(Nc4ccc(Oc5ccc6c(c5)nnn6C)c(F)c4F)c3n2)CC1] |
| 45 | 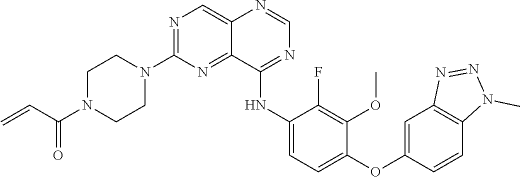 [SMILES: C=CC(=O)N1CCN(c2ncc3ncnc(Nc4ccc(Oc5ccc6c(c5)nnn6C)c(OC)c4F)c3n2)CC1] |
| 46 | 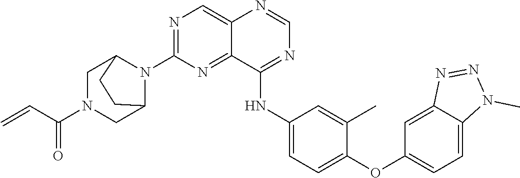 [SMILES: C=CC(=O)N1CC2CCC(C1)N2c1ncc2ncnc(Nc3ccc(Oc4ccc5c(c4)nnn5C)c(C)c3)c2n1] |
| 47 | 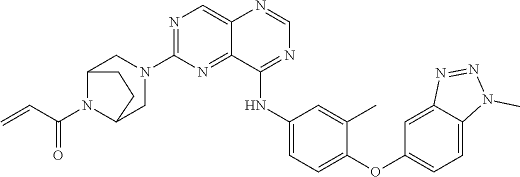 [SMILES: C=CC(=O)N1C2CCC1CN(c1ncc3ncnc(Nc4ccc(Oc5ccc6c(c5)nnn6C)c(C)c4)c3n1)C2] |
| 48 | 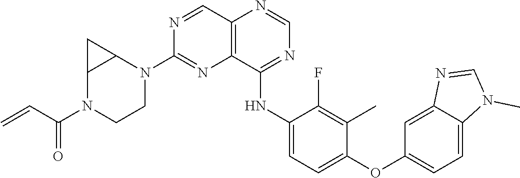 [SMILES: C=CC(=O)N1CCN(c2ncc3ncnc(Nc4ccc(Oc5ccc6c(c5)ncn6C)c(C)c4F)c3n2)C2CC21] |
| 49 | 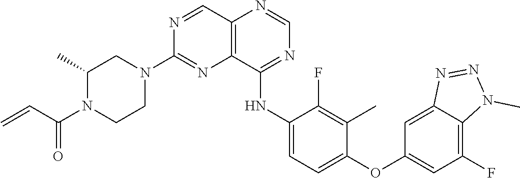 [SMILES: C=CC(=O)N1CCN(c2ncc3ncnc(Nc4ccc(Oc5cc(F)c6c(c5)nnn6C)c(C)c4F)c3n2)C[C@H]1C] |
| 50 | 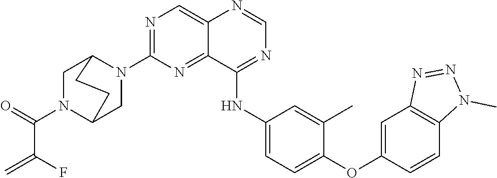 [SMILES: C=C(F)C(=O)N1CC2CCC1CN2c1ncc2ncnc(Nc3ccc(Oc4ccc5c(c4)nnn5C)c(C)c3)c2n1] |
| 51 | 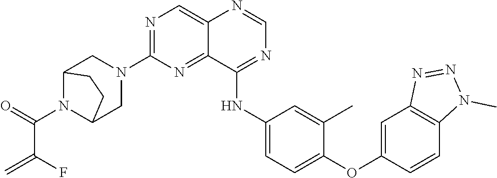 [SMILES: C=C(F)C(=O)N1C2CCC1CN(c1ncc3ncnc(Nc4ccc(Oc5ccc6c(c5)nnn6C)c(C)c4)c3n1)C2] |
| 52 | 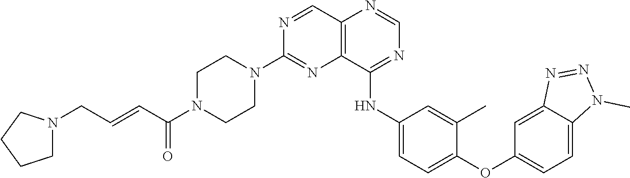 [SMILES: Cc1cc(Nc2ncnc3cnc(N4CCN(C(=O)/C=C/CN5CCCC5)CC4)nc23)ccc1Oc1ccc2c(c1)nnn2C] |
| 53 | 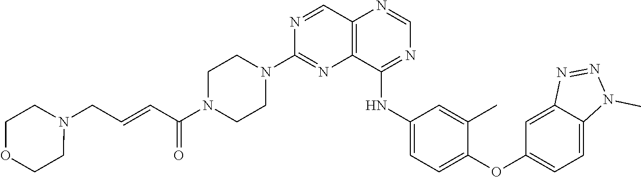 [SMILES: Cc1cc(Nc2ncnc3cnc(N4CCN(C(=O)/C=C/CN5CCOCC5)CC4)nc23)ccc1Oc1ccc2c(c1)nnn2C] |
| 54 | 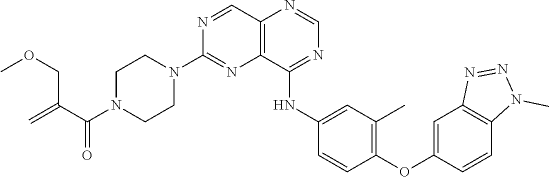 |
| 55 | 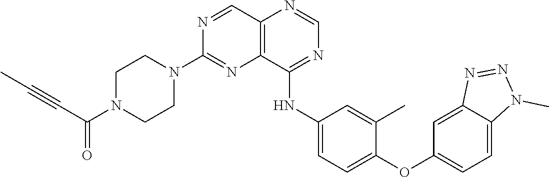 [SMILES: CC#CC(=O)N1CCN(c2ncc3ncnc(Nc4ccc(Oc5ccc6c(c5)nnn6C)c(C)c4)c3n2)CC1] |
| 56 | 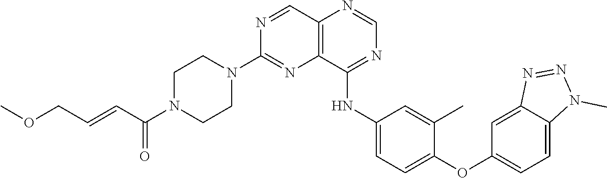 |
| 57 | 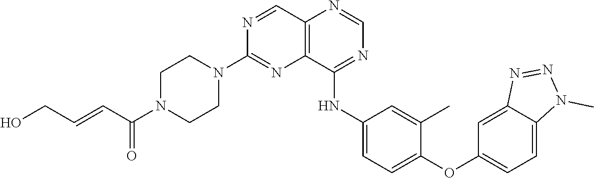 [SMILES: Cc1cc(Nc2ncnc3cnc(N4CCN(C(=O)/C=C/CO)CC4)nc23)ccc1Oc1ccc2c(c1)nnn2C] |
| 58 | 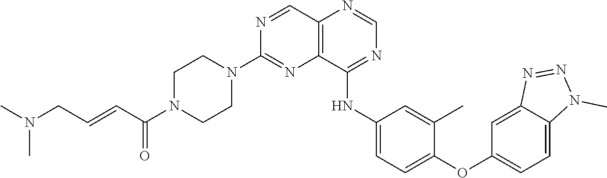 |
| 59 | 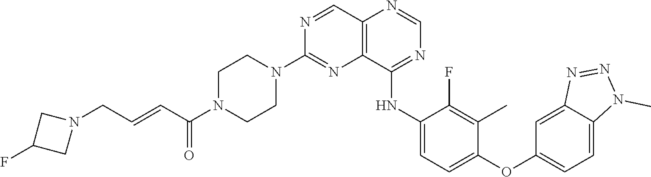 [SMILES: Cc1c(Oc2ccc3c(c2)nnn3C)ccc(Nc2ncnc3cnc(N4CCN(C(=O)/C=C/CN5CC(F)C5)CC4)nc23)c1F] |
| 60 | 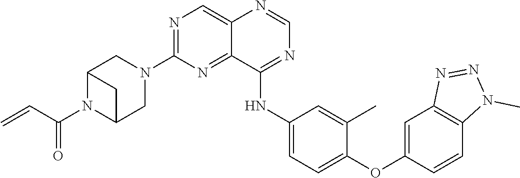 [SMILES: C=CC(=O)N1C2CC1CN(c1ncc3ncnc(Nc4ccc(Oc5ccc6c(c5)nnn6C)c(C)c4)c3n1)C2] |
| 61 | 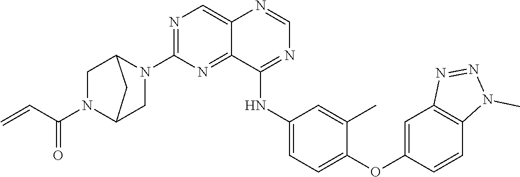 [SMILES: C=CC(=O)N1CC2CC1CN2c1ncc2ncnc(Nc3ccc(Oc4ccc5c(c4)nnn5C)c(C)c3)c2n1] |
| 62 | 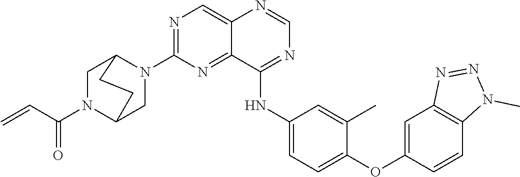 [SMILES: C=CC(=O)N1CC2CCC1CN2c1ncc2ncnc(Nc3ccc(Oc4ccc5c(c4)nnn5C)c(C)c3)c2n1] |
| 63 | 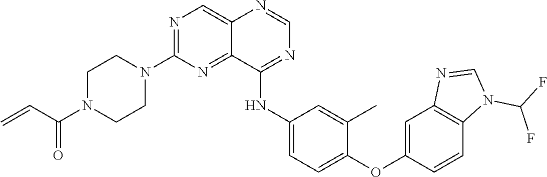 [SMILES: C=CC(=O)N1CCN(c2ncc3ncnc(Nc4ccc(Oc5ccc6c(c5)ncn6C(F)F)c(C)c4)c3n2)CC1] |
| 64 | 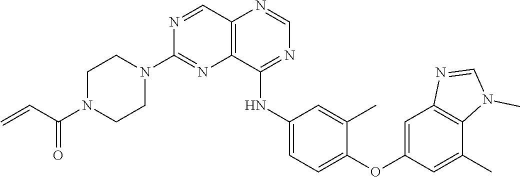 [SMILES: C=CC(=O)N1CCN(c2ncc3ncnc(Nc4ccc(Oc5cc(C)c6c(c5)ncn6C)c(C)c4)c3n2)CC1] |
| 65 | 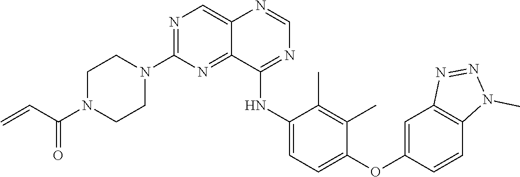 [SMILES: C=CC(=O)N1CCN(c2ncc3ncnc(Nc4ccc(Oc5ccc6c(c5)nnn6C)c(C)c4C)c3n2)CC1] |
| 66 | 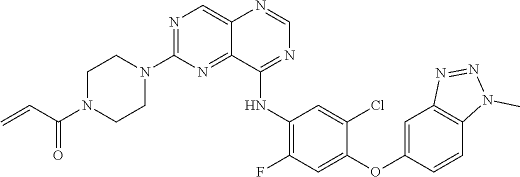 [SMILES: C=CC(=O)N1CCN(c2ncc3ncnc(Nc4cc(Cl)c(Oc5ccc6c(c5)nnn6C)cc4F)c3n2)CC1] |
| 67 | 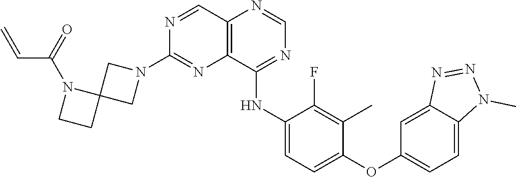 [SMILES: C=CC(=O)N1CCC12CN(c1ncc3ncnc(Nc4ccc(Oc5ccc6c(c5)nnn6C)c(C)c4F)c3n1)C2] |
| 68 | 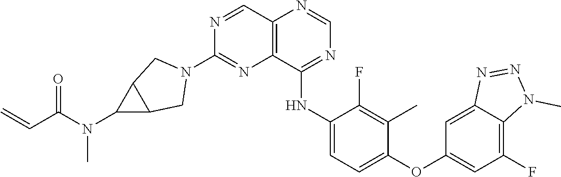 [SMILES: C=CC(=O)N(C)C1C2CN(c3ncc4ncnc(Nc5ccc(Oc6cc(F)c7c(c6)nnn7C)c(C)c5F)c4n3)CC21] |
| 69 | 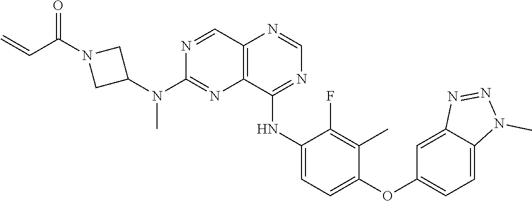 [SMILES: C=CC(=O)N1CC(N(C)c2ncc3ncnc(Nc4ccc(Oc5ccc6c(c5)nnn6C)c(C)c4F)c3n2)C1] |
| 70 | 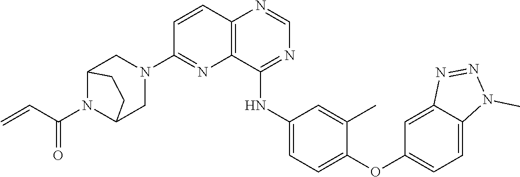 [SMILES: C=CC(=O)N1C2CCC1CN(c1ccc3ncnc(Nc4ccc(Oc5ccc6c(c5)nnn6C)c(C)c4)c3n1)C2] |
| 71 | 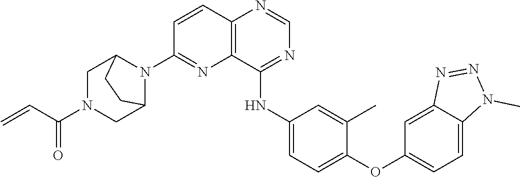 [SMILES: C=CC(=O)N1CC2CCC(C1)N2c1ccc2ncnc(Nc3ccc(Oc4ccc5c(c4)nnn5C)c(C)c3)c2n1] |
| 72 | 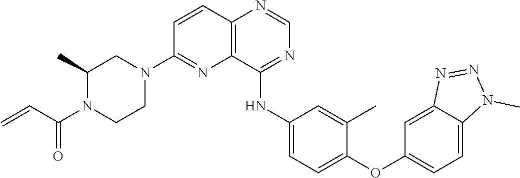 [SMILES: C=CC(=O)N1CCN(c2ccc3ncnc(Nc4ccc(Oc5ccc6c(c5)nnn6C)c(C)c4)c3n2)C[C@@H]1C] |
| 73 | 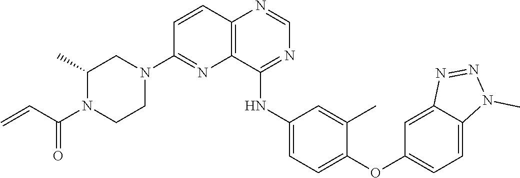 [SMILES: C=CC(=O)N1CCN(c2ccc3ncnc(Nc4ccc(Oc5ccc6c(c5)nnn6C)c(C)c4)c3n2)C[C@H]1C] |
| 74 | 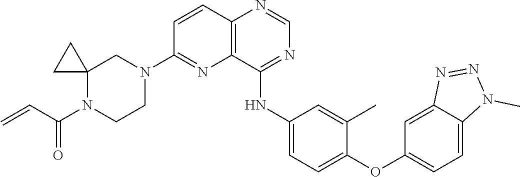 [SMILES: C=CC(=O)N1CCN(c2ccc3ncnc(Nc4ccc(Oc5ccc6c(c5)nnn6C)c(C)c4)c3n2)CC12CC2] |
| 75 | 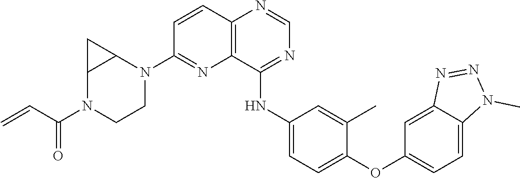 [SMILES: C=CC(=O)N1CCN(c2ccc3ncnc(Nc4ccc(Oc5ccc6c(c5)nnn6C)c(C)c4)c3n2)C2CC21] |
| 76 | 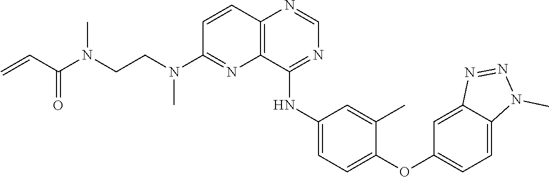 [SMILES: C=CC(=O)N(C)CCN(C)c1ccc2ncnc(Nc3ccc(Oc4ccc5c(c4)nnn5C)c(C)c3)c2n1] |
| 77 | 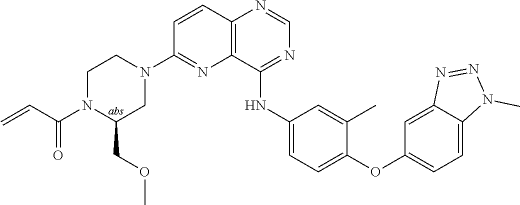 [SMILES: C=CC(=O)N1CCN(c2ccc3ncnc(Nc4ccc(Oc5ccc6c(c5)nnn6C)c(C)c4)c3n2)C[C@H]1COC] |
| 78 | 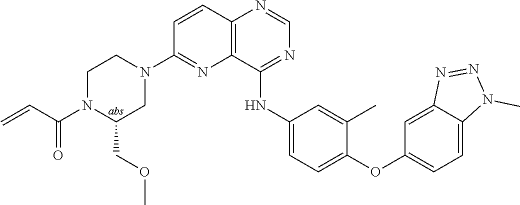 [SMILES: C=CC(=O)N1CCN(c2ccc3ncnc(Nc4ccc(Oc5ccc6c(c5)nnn6C)c(C)c4)c3n2)C[C@@H]1COC] |
| 79 | 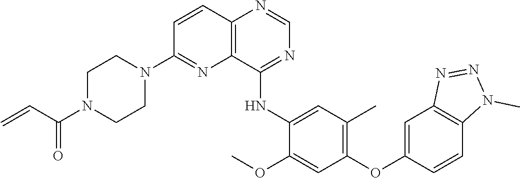 [SMILES: C=CC(=O)N1CCN(c2ccc3ncnc(Nc4cc(C)c(Oc5ccc6c(c5)nnn6C)cc4OC)c3n2)CC1] |
| 80 | 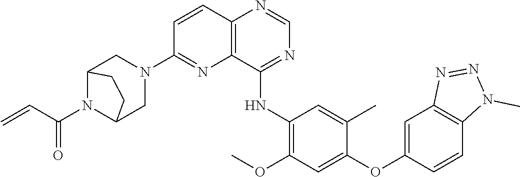 [SMILES: C=CC(=O)N1C2CCC1CN(c1ccc3ncnc(Nc4cc(C)c(Oc5ccc6c(c5)nnn6C)cc4OC)c3n1)C2] |
| 81 | 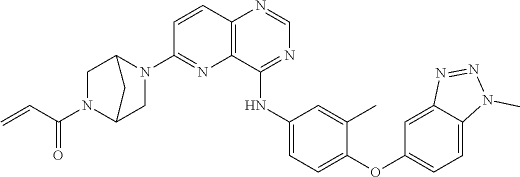 [SMILES: C=CC(=O)N1CC2CC1CN2c1ccc2ncnc(Nc3ccc(Oc4ccc5c(c4)nnn5C)c(C)c3)c2n1] |
| 82 | 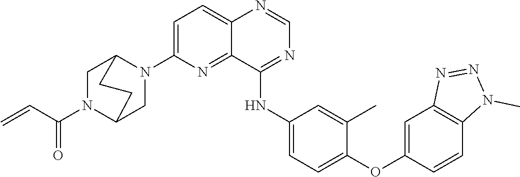 [SMILES: C=CC(=O)N1CC2CCC1CN2c1ccc2ncnc(Nc3ccc(Oc4ccc5c(c4)nnn5C)c(C)c3)c2n1] |
| 83 | 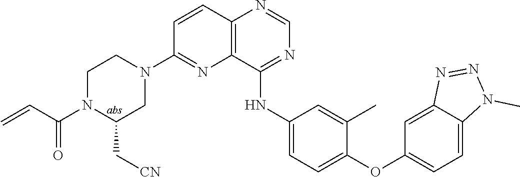 [SMILES: C=CC(=O)N1CCN(c2ccc3ncnc(Nc4ccc(Oc5ccc6c(c5)nnn6C)c(C)c4)c3n2)C[C@@H]1CC#N] |
| 84 | 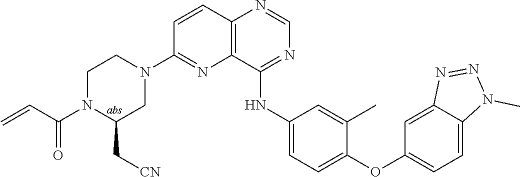 [SMILES: C=CC(=O)N1CCN(c2ccc3ncnc(Nc4ccc(Oc5ccc6c(c5)nnn6C)c(C)c4)c3n2)C[C@H]1CC#N] |
| 85 | 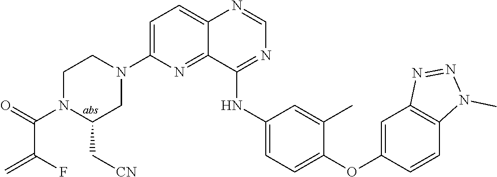 [SMILES: C=C(F)C(=O)N1CCN(c2ccc3ncnc(Nc4ccc(Oc5ccc6c(c5)nnn6C)c(C)c4)c3n2)C[C@@H]1CC#N] |
| 86 | 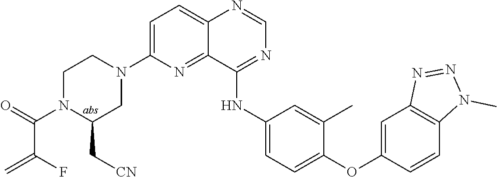 [SMILES: C=C(F)C(=O)N1CCN(c2ccc3ncnc(Nc4ccc(Oc5ccc6c(c5)nnn6C)c(C)c4)c3n2)C[C@H]1CC#N] |
| 87 | 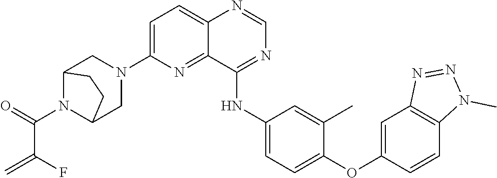 [SMILES: C=C(F)C(=O)N1C2CCC1CN(c1ccc3ncnc(Nc4ccc(Oc5ccc6c(c5)nnn6C)c(C)c4)c3n1)C2] |
| 88 | 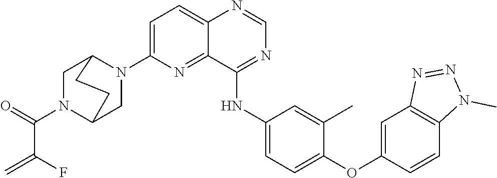 [SMILES: C=C(F)C(=O)N1CC2CCC1CN2c1ccc2ncnc(Nc3ccc(Oc4ccc5c(c4)nnn5C)c(C)c3)c2n1] |
| 89 | 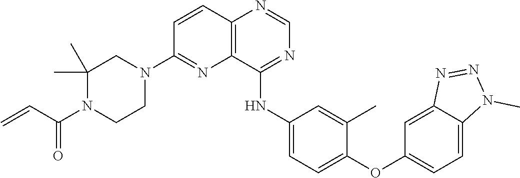 [SMILES: C=CC(=O)N1CCN(c2ccc3ncnc(Nc4ccc(Oc5ccc6c(c5)nnn6C)c(C)c4)c3n2)CC1(C)C] |
| 90 | 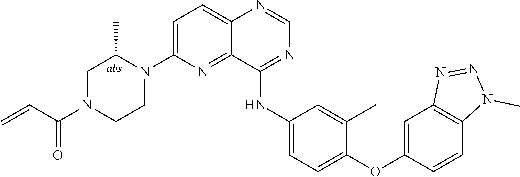 [SMILES: C=CC(=O)N1CCN(c2ccc3ncnc(Nc4ccc(Oc5ccc6c(c5)nnn6C)c(C)c4)c3n2)[C@@H](C)C1] |
| 91 | 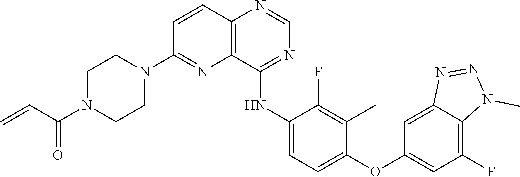 [SMILES: C=CC(=O)N1CCN(c2ccc3ncnc(Nc4ccc(Oc5cc(F)c6c(c5)nnn6C)c(C)c4F)c3n2)CC1] |
| 92 | 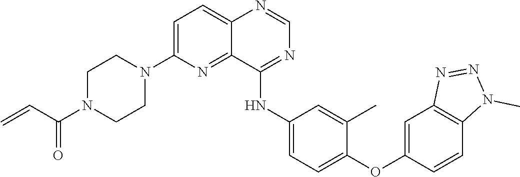 [SMILES: C=CC(=O)N1CCN(c2ccc3ncnc(Nc4ccc(Oc5ccc6c(c5)nnn6C)c(C)c4)c3n2)CC1] |
| 93 | 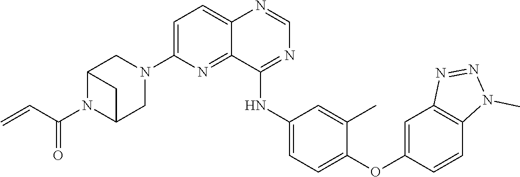 [SMILES: C=CC(=O)N1C2CC1CN(c1ccc3ncnc(Nc4ccc(Oc5ccc6c(c5)nnn6C)c(C)c4)c3n1)C2] |
| 94 | 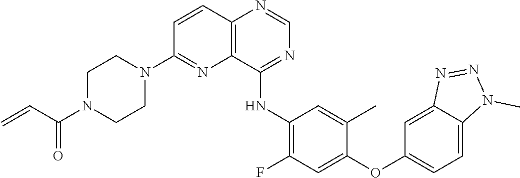 [SMILES: C=CC(=O)N1CCN(c2ccc3ncnc(Nc4cc(C)c(Oc5ccc6c(c5)nnn6C)cc4F)c3n2)CC1] |
| 95 | 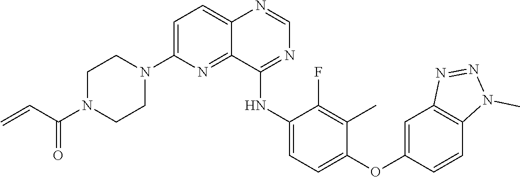 [SMILES: C=CC(=O)N1CCN(c2ccc3ncnc(Nc4ccc(Oc5ccc6c(c5)nnn6C)c(C)c4F)c3n2)CC1] |
| 96 | 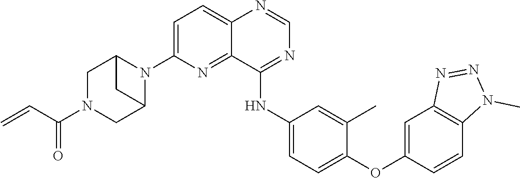 [SMILES: C=CC(=O)N1CC2CC(C1)N2c1ccc2ncnc(Nc3ccc(Oc4ccc5c(c4)nnn5C)c(C)c3)c2n1] |
| 97 | 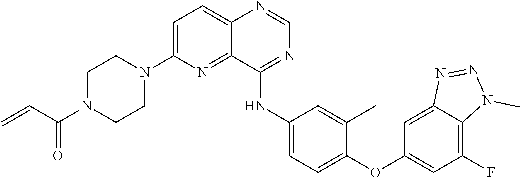 [SMILES: C=CC(=O)N1CCN(c2ccc3ncnc(Nc4ccc(Oc5cc(F)c6c(c5)nnn6C)c(C)c4)c3n2)CC1] |
| 98 | 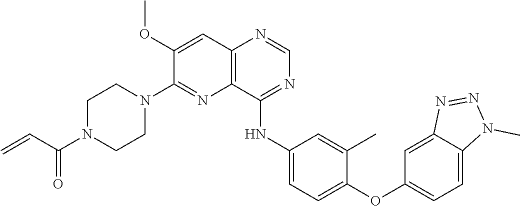 [SMILES: C=CC(=O)N1CCN(c2nc3c(Nc4ccc(Oc5ccc6c(c5)nnn6C)c(C)c4)ncnc3cc2OC)CC1] |
| 99 | 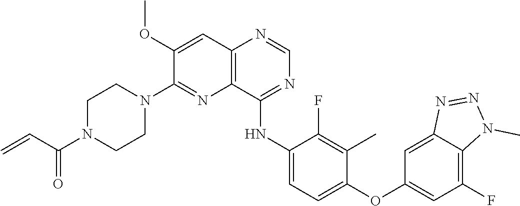 [SMILES: C=CC(=O)N1CCN(c2nc3c(Nc4ccc(Oc5cc(F)c6c(c5)nnn6C)c(C)c4F)ncnc3cc2OC)CC1] |
| 100 | 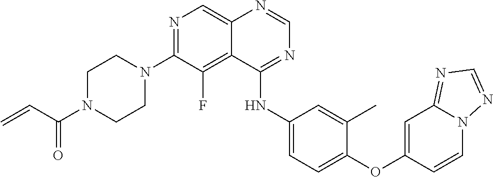 [SMILES: C=CC(=O)N1CCN(c2ncc3ncnc(Nc4ccc(Oc5ccn6ncnc6c5)c(C)c4)c3c2F)CC1] |
| 101 | 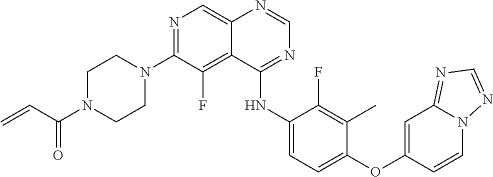 [SMILES: C=CC(=O)N1CCN(c2ncc3ncnc(Nc4ccc(Oc5ccn6ncnc6c5)c(C)c4F)c3c2F)CC1] |
| 102 | 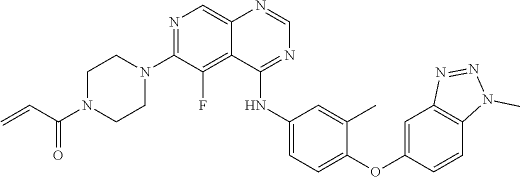 [SMILES: C=CC(=O)N1CCN(c2ncc3ncnc(Nc4ccc(Oc5ccc6c(c5)nnn6C)c(C)c4)c3c2F)CC1] |
| 103 | 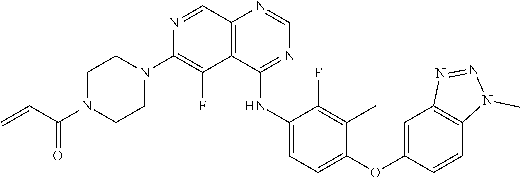 [SMILES: C=CC(=O)N1CCN(c2ncc3ncnc(Nc4ccc(Oc5ccc6c(c5)nnn6C)c(C)c4F)c3c2F)CC1] |
| 104 | 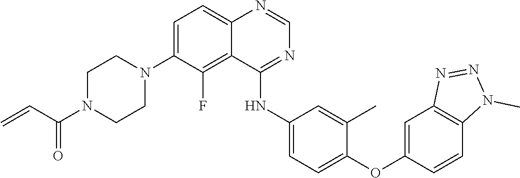 [SMILES: C=CC(=O)N1CCN(c2ccc3ncnc(Nc4ccc(Oc5ccc6c(c5)nnn6C)c(C)c4)c3c2F)CC1] |
| 105 | 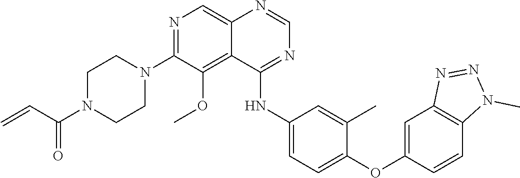 [SMILES: C=CC(=O)N1CCN(c2ncc3ncnc(Nc4ccc(Oc5ccc6c(c5)nnn6C)c(C)c4)c3c2OC)CC1] |
| 106 | 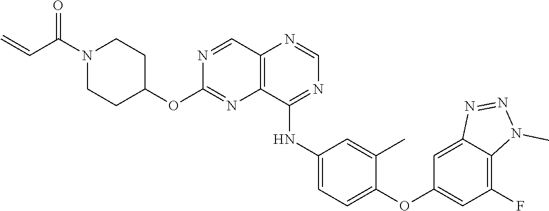 [SMILES: C=CC(=O)N1CCC(Oc2ncc3ncnc(Nc4ccc(Oc5cc(F)c6c(c5)nnn6C)c(C)c4)c3n2)CC1] |
| 107 | 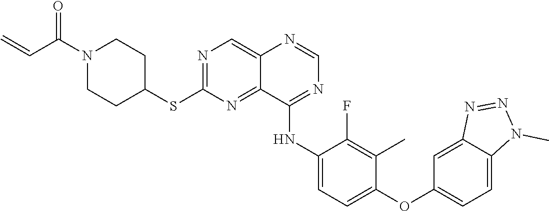 [SMILES: C=CC(=O)N1CCC(Sc2ncc3ncnc(Nc4ccc(Oc5ccc6c(c5)nnn6C)c(C)c4F)c3n2)CC1] |
| 108 | 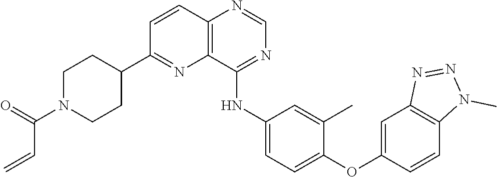 [SMILES: C=CC(=O)N1CCC(c2ccc3ncnc(Nc4ccc(Oc5ccc6c(c5)nnn6C)c(C)c4)c3n2)CC1] |
| 109 | 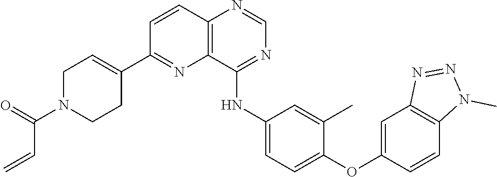 [SMILES: C=CC(=O)N1CC=C(c2ccc3ncnc(Nc4ccc(Oc5ccc6c(c5)nnn6C)c(C)c4)c3n2)CC1] |
| 110 | 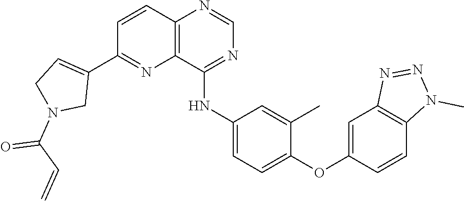 [SMILES: C=CC(=O)N1CC=C(c2ccc3ncnc(Nc4ccc(Oc5ccc6c(c5)nnn6C)c(C)c4)c3n2)C1] |
| 111 | 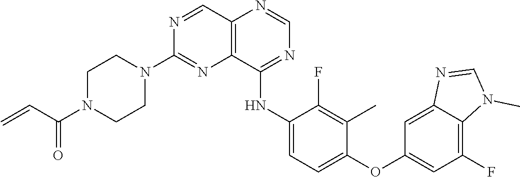 [SMILES: C=CC(=O)N1CCN(c2ncc3ncnc(Nc4ccc(Oc5cc(F)c6c(c5)ncn6C)c(C)c4F)c3n2)CC1] |
| 112 | 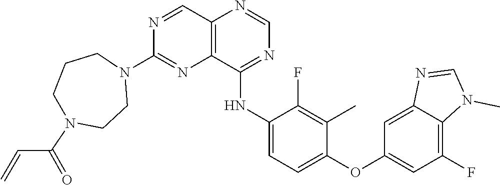 [SMILES: C=CC(=O)N1CCCN(c2ncc3ncnc(Nc4ccc(Oc5cc(F)c6c(c5)ncn6C)c(C)c4F)c3n2)CC1] |
| 113 | 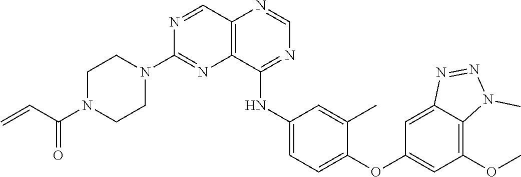 [SMILES: C=CC(=O)N1CCN(c2ncc3ncnc(Nc4ccc(Oc5cc(OC)c6c(c5)nnn6C)c(C)c4)c3n2)CC1] |
| 114 | 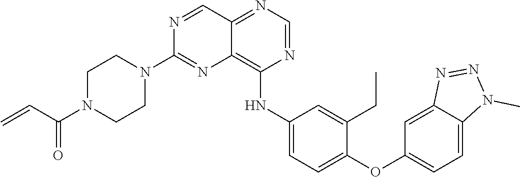 [SMILES: C=CC(=O)N1CCN(c2ncc3ncnc(Nc4ccc(Oc5ccc6c(c5)nnn6C)c(CC)c4)c3n2)CC1] |
| 115 | 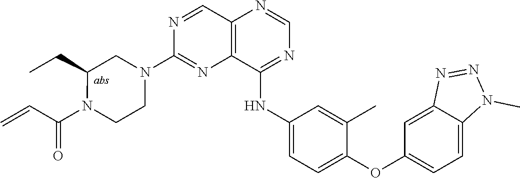 [SMILES: C=CC(=O)N1CCN(c2ncc3ncnc(Nc4ccc(Oc5ccc6c(c5)nnn6C)c(C)c4)c3n2)C[C@@H]1CC] |
| 116 | 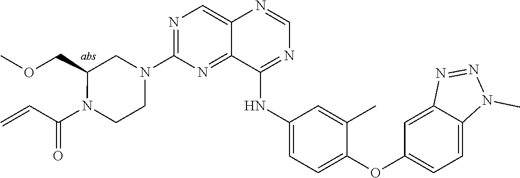 [SMILES: C=CC(=O)N1CCN(c2ncc3ncnc(Nc4ccc(Oc5ccc6c(c5)nnn6C)c(C)c4)c3n2)C[C@@H]1COC] |
| 117 | 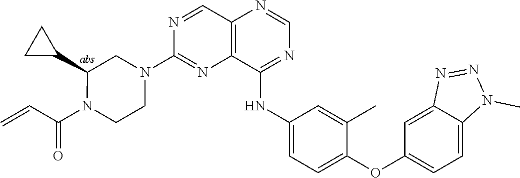 [SMILES: C=CC(=O)N1CCN(c2ncc3ncnc(Nc4ccc(Oc5ccc6c(c5)nnn6C)c(C)c4)c3n2)C[C@@H]1C1CC1] |
| 118 | 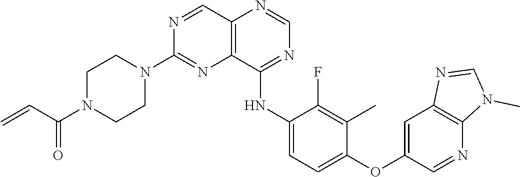 [SMILES: C=CC(=O)N1CCN(c2ncc3ncnc(Nc4ccc(Oc5cnc6c(c5)ncn6C)c(C)c4F)c3n2)CC1] |
| 119 | 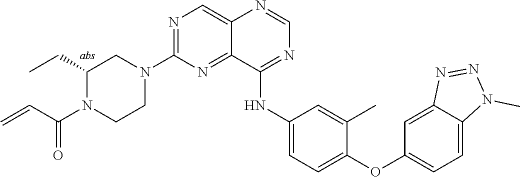 [SMILES: C=CC(=O)N1CCN(c2ncc3ncnc(Nc4ccc(Oc5ccc6c(c5)nnn6C)c(C)c4)c3n2)C[C@H]1CC] |
| 120 | 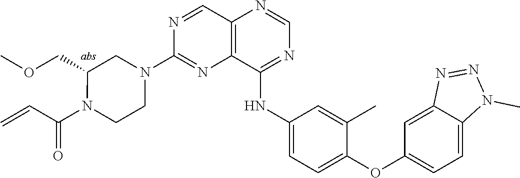 [SMILES: C=CC(=O)N1CCN(c2ncc3ncnc(Nc4ccc(Oc5ccc6c(c5)nnn6C)c(C)c4)c3n2)C[C@H]1COC] |
| 121 | 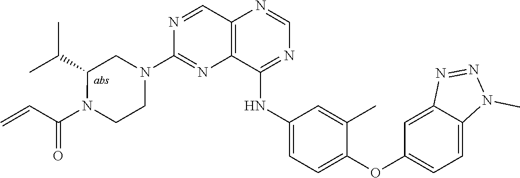 [SMILES: C=CC(=O)N1CCN(c2ncc3ncnc(Nc4ccc(Oc5ccc6c(c5)nnn6C)c(C)c4)c3n2)C[C@H]1C(C)C] |
| 122 | 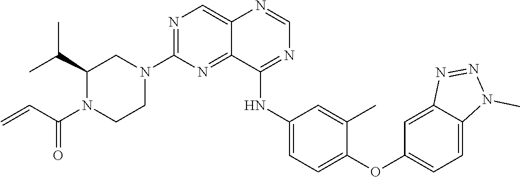 [SMILES: C=CC(=O)N1CCN(c2ncc3ncnc(Nc4ccc(Oc5ccc6c(c5)nnn6C)c(C)c4)c3n2)C[C@@H]1C(C)C] |
| 123 | 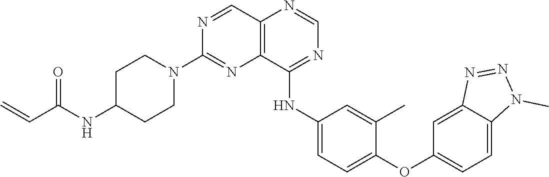 [SMILES: C=CC(=O)NC1CCN(c2ncc3ncnc(Nc4ccc(Oc5ccc6c(c5)nnn6C)c(C)c4)c3n2)CC1] |
| 124 | 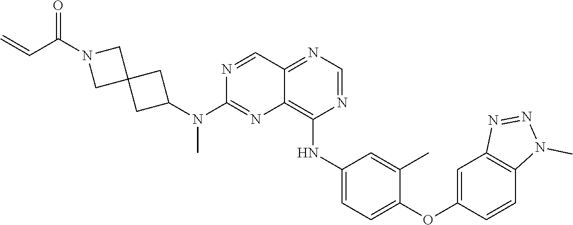 [SMILES: C=CC(=O)N1CC2(CC(N(C)c3ncc4ncnc(Nc5ccc(Oc6ccc7c(c6)nnn7C)c(C)c5)c4n3)C2)C1] |
| 125 | 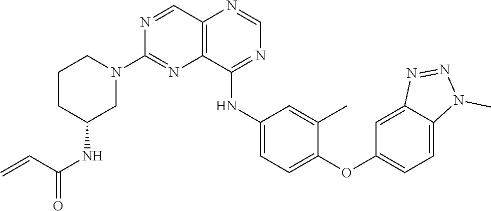 [SMILES: C=CC(=O)N[C@@H]1CCCN(c2ncc3ncnc(Nc4ccc(Oc5ccc6c(c5)nnn6C)c(C)c4)c3n2)C1] |
| 126 | 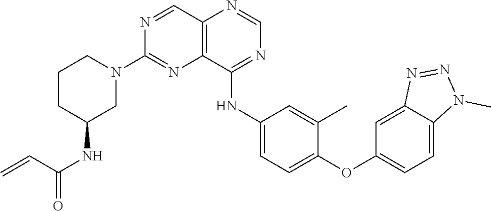 [SMILES: C=CC(=O)N[C@H]1CCCN(c2ncc3ncnc(Nc4ccc(Oc5ccc6c(c5)nnn6C)c(C)c4)c3n2)C1] |
| 127 | 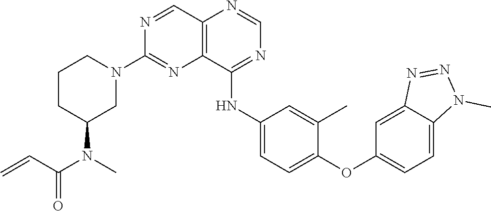 [SMILES: C=CC(=O)N(C)[C@H]1CCCN(c2ncc3ncnc(Nc4ccc(Oc5ccc6c(c5)nnn6C)c(C)c4)c3n2)C1] |
| 128 | 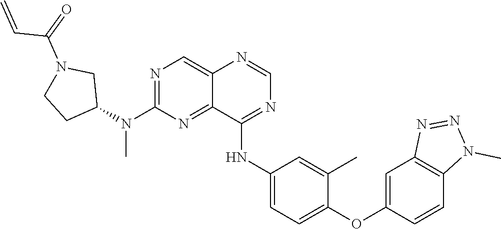 [SMILES: C=CC(=O)N1CC[C@@H](N(C)c2ncc3ncnc(Nc4ccc(Oc5ccc6c(c5)nnn6C)c(C)c4)c3n2)C1] |
| 129 | 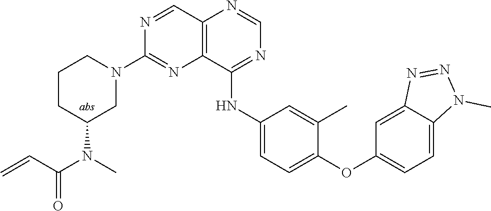 [SMILES: C=CC(=O)N(C)[C@@H]1CCCN(c2ncc3ncnc(Nc4ccc(Oc5ccc6c(c5)nnn6C)c(C)c4)c3n2)C1] |
| 130 | 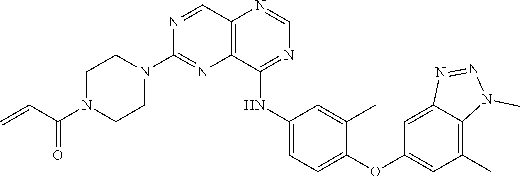 [SMILES: C=CC(=O)N1CCN(c2ncc3ncnc(Nc4ccc(Oc5cc(C)c6c(c5)nnn6C)c(C)c4)c3n2)CC1] |
| 131 | 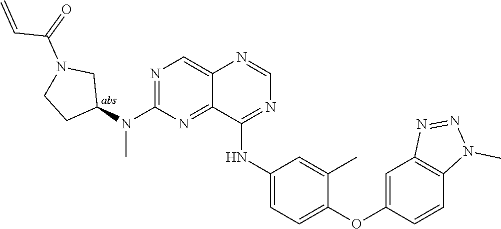 [SMILES: C=CC(=O)N1CC[C@H](N(C)c2ncc3ncnc(Nc4ccc(Oc5ccc6c(c5)nnn6C)c(C)c4)c3n2)C1] |
| 132 | 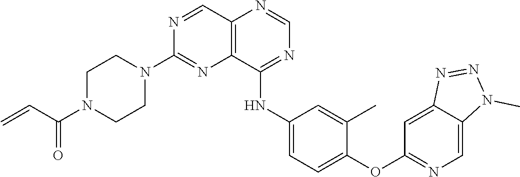 [SMILES: C=CC(=O)N1CCN(c2ncc3ncnc(Nc4ccc(Oc5cc6nnn(C)c6cn5)c(C)c4)c3n2)CC1] |
| 133 | 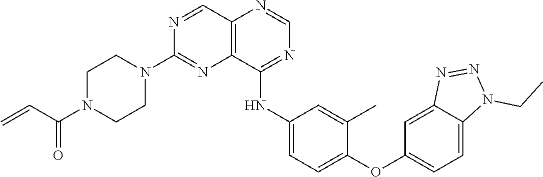 [SMILES: C=CC(=O)N1CCN(c2ncc3ncnc(Nc4ccc(Oc5ccc6c(c5)nnn6CC)c(C)c4)c3n2)CC1] |
| 134 | 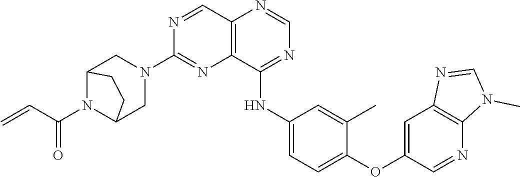 [SMILES: C=CC(=O)N1C2CCC1CN(c1ncc3ncnc(Nc4ccc(Oc5cnc6c(c5)ncn6C)c(C)c4)c3n1)C2] |
| 135 | 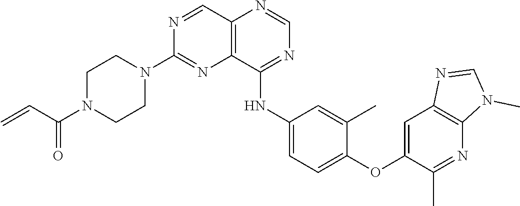 [SMILES: C=CC(=O)N1CCN(c2ncc3ncnc(Nc4ccc(Oc5cc6ncn(C)c6nc5C)c(C)c4)c3n2)CC1] |
| 136 | 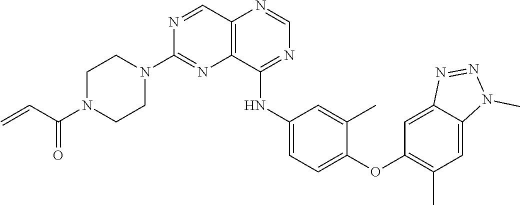 [SMILES: C=CC(=O)N1CCN(c2ncc3ncnc(Nc4ccc(Oc5cc6nnn(C)c6cc5C)c(C)c4)c3n2)CC1] |
| 137 | 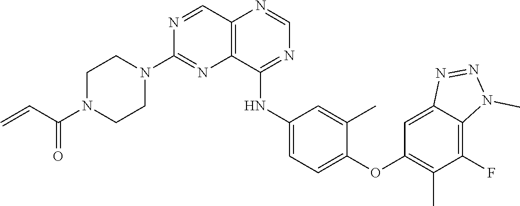 [SMILES: C=CC(=O)N1CCN(c2ncc3ncnc(Nc4ccc(Oc5cc6nnn(C)c6c(F)c5C)c(C)c4)c3n2)CC1] |
| 138 | 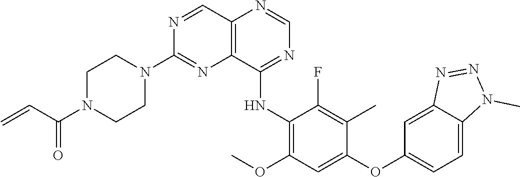 [SMILES: C=CC(=O)N1CCN(c2ncc3ncnc(Nc4c(OC)cc(Oc5ccc6c(c5)nnn6C)c(C)c4F)c3n2)CC1] |
| 139 | 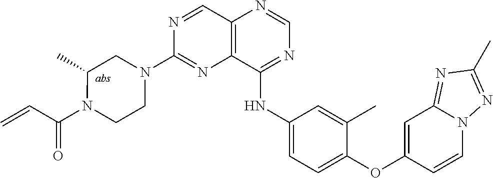 [SMILES: C=CC(=O)N1CCN(c2ncc3ncnc(Nc4ccc(Oc5ccn6nc(C)nc6c5)c(C)c4)c3n2)C[C@H]1C] |
| 140 | 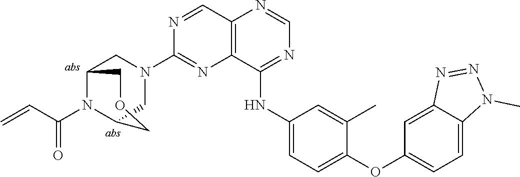 [SMILES: C=CC(=O)N1[C@@H]2COC[C@H]1CN(c1ncc3ncnc(Nc4ccc(Oc5ccc6c(c5)nnn6C)c(C)c4)c3n1)C2] |
| 141 | 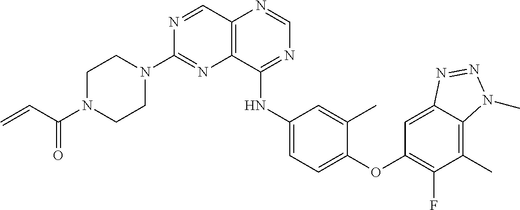 [SMILES: C=CC(=O)N1CCN(c2ncc3ncnc(Nc4ccc(Oc5cc6nnn(C)c6c(C)c5F)c(C)c4)c3n2)CC1] |
| 142 | 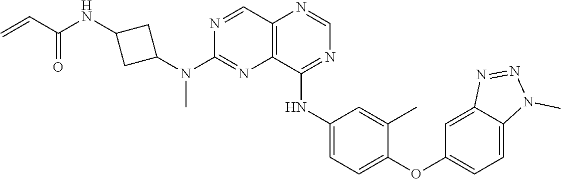 [SMILES: C=CC(=O)NC1CC(N(C)c2ncc3ncnc(Nc4ccc(Oc5ccc6c(c5)nnn6C)c(C)c4)c3n2)C1] |
| 143 | 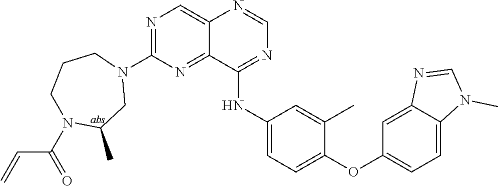 [SMILES: C=CC(=O)N1CCCN(c2ncc3ncnc(Nc4ccc(Oc5ccc6c(c5)ncn6C)c(C)c4)c3n2)C[C@H]1C] |
| 144 | 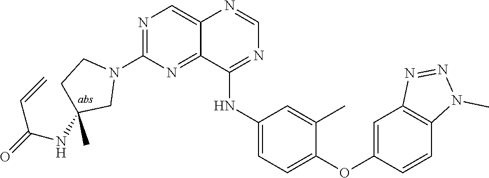 [SMILES: C=CC(=O)N[C@]1(C)CCN(c2ncc3ncnc(Nc4ccc(Oc5ccc6c(c5)nnn6C)c(C)c4)c3n2)C1] |
| 145 | 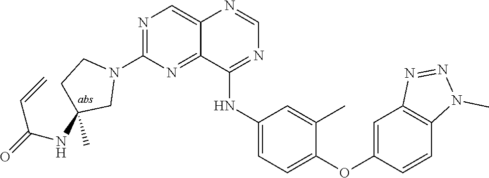 [SMILES: C=CC(=O)N[C@@]1(C)CCN(c2ncc3ncnc(Nc4ccc(Oc5ccc6c(c5)nnn6C)c(C)c4)c3n2)C1] |
| 146 | 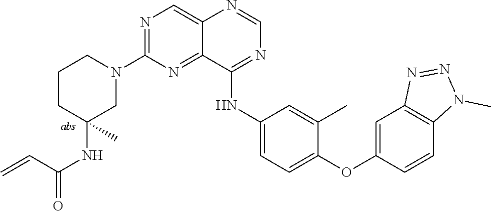 [SMILES: C=CC(=O)N[C@@]1(C)CCCN(c2ncc3ncnc(Nc4ccc(Oc5ccc6c(c5)nnn6C)c(C)c4)c3n2)C1] |
| 147 | 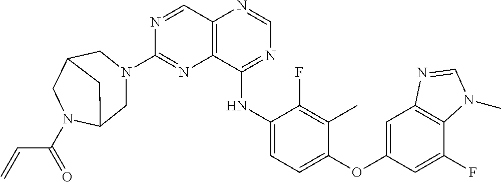 [SMILES: C=CC(=O)N1CC2CC1CN(c1ncc3ncnc(Nc4ccc(Oc5cc(F)c6c(c5)ncn6C)c(C)c4F)c3n1)C2] |
| 148 | 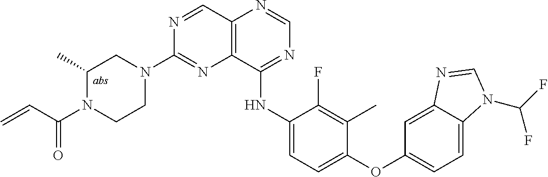 [SMILES: C=CC(=O)N1CCN(c2ncc3ncnc(Nc4ccc(Oc5ccc6c(c5)ncn6C(F)F)c(C)c4F)c3n2)C[C@H]1C] |
| 149 | 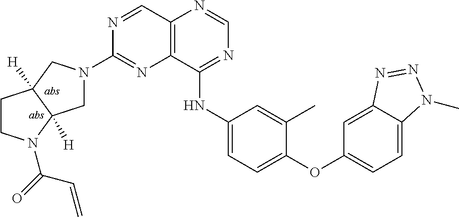 [SMILES: C=CC(=O)N1CC[C@H]2CN(c3ncc4ncnc(Nc5ccc(Oc6ccc7c(c6)nnn7C)c(C)c5)c4n3)C[C@H]21] |
| 150 | 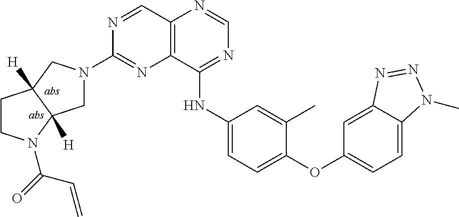 [SMILES: C=CC(=O)N1CC[C@@H]2CN(c3ncc4ncnc(Nc5ccc(Oc6ccc7c(c6)nnn7C)c(C)c5)c4n3)C[C@@H]21] |
| 151 | 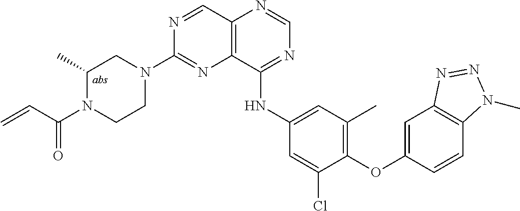 [SMILES: C=CC(=O)N1CCN(c2ncc3ncnc(Nc4cc(C)c(Oc5ccc6c(c5)nnn6C)c(Cl)c4)c3n2)C[C@H]1C] |
| 152 | 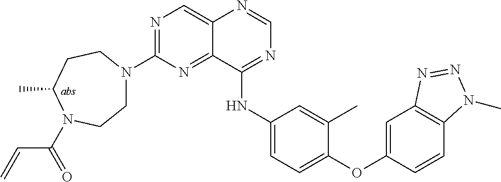 [SMILES: C=CC(=O)N1CCN(c2ncc3ncnc(Nc4ccc(Oc5ccc6c(c5)nnn6C)c(C)c4)c3n2)CC[C@H]1C] |
| 153 | 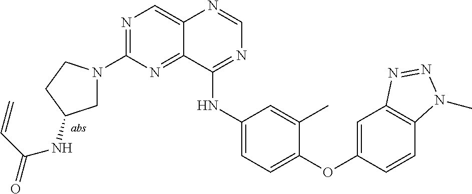 [SMILES: C=CC(=O)N[C@@H]1CCN(c2ncc3ncnc(Nc4ccc(Oc5ccc6c(c5)nnn6C)c(C)c4)c3n2)C1] |
| 154 | 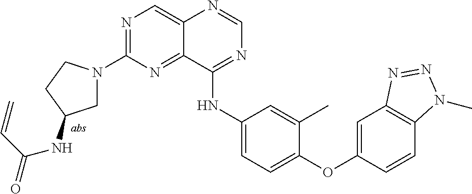 [SMILES: C=CC(=O)N[C@H]1CCN(c2ncc3ncnc(Nc4ccc(Oc5ccc6c(c5)nnn6C)c(C)c4)c3n2)C1] |
| 155 | 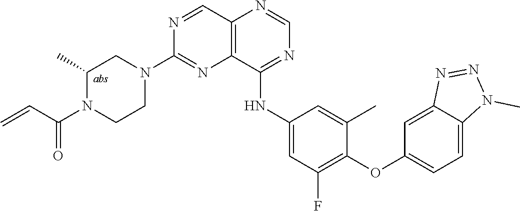 [SMILES: C=CC(=O)N1CCN(c2ncc3ncnc(Nc4cc(C)c(Oc5ccc6c(c5)nnn6C)c(F)c4)c3n2)C[C@H]1C] |
| 156 | 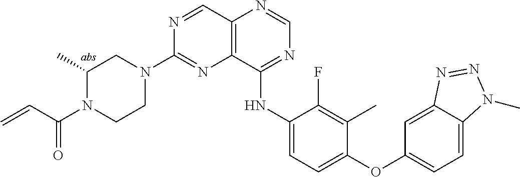 [SMILES: C=CC(=O)N1CCN(c2ncc3ncnc(Nc4ccc(Oc5ccc6c(c5)nnn6C)c(C)c4F)c3n2)C[C@H]1C] |
| 157 | 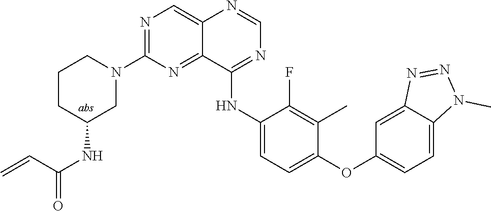 [SMILES: C=CC(=O)N[C@@H]1CCCN(c2ncc3ncnc(Nc4ccc(Oc5ccc6c(c5)nnn6C)c(C)c4F)c3n2)C1] |
| 158 | 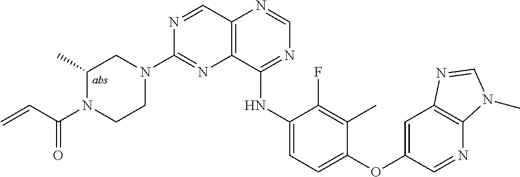 [SMILES: C=CC(=O)N1CCN(c2ncc3ncnc(Nc4ccc(Oc5cnc6c(c5)ncn6C)c(C)c4F)c3n2)C[C@H]1C] |
| 159 | 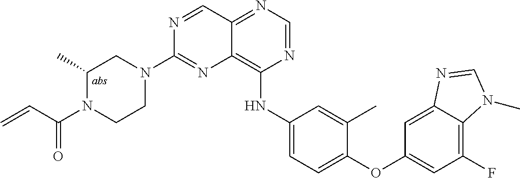 [SMILES: C=CC(=O)N1CCN(c2ncc3ncnc(Nc4ccc(Oc5cc(F)c6c(c5)ncn6C)c(C)c4)c3n2)C[C@H]1C] |
| 160 | 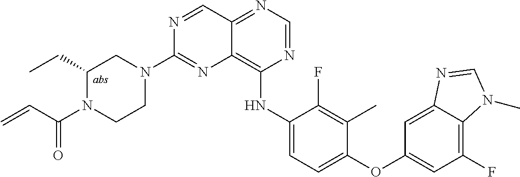 [SMILES: C=CC(=O)N1CCN(c2ncc3ncnc(Nc4ccc(Oc5cc(F)c6c(c5)ncn6C)c(C)c4F)c3n2)C[C@H]1CC] |
| 161 | 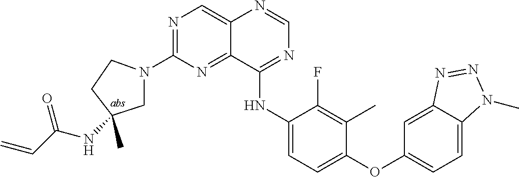 [SMILES: C=CC(=O)N[C@]1(C)CCN(c2ncc3ncnc(Nc4ccc(Oc5ccc6c(c5)nnn6C)c(C)c4F)c3n2)C1] |
| 162 | 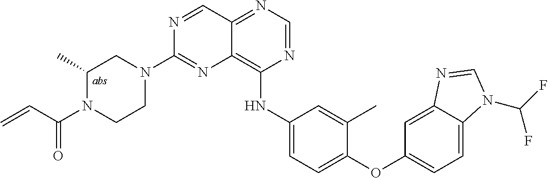 [SMILES: C=CC(=O)N1CCN(c2ncc3ncnc(Nc4ccc(Oc5ccc6c(c5)ncn6C(F)F)c(C)c4)c3n2)C[C@H]1C] |
| 163 | 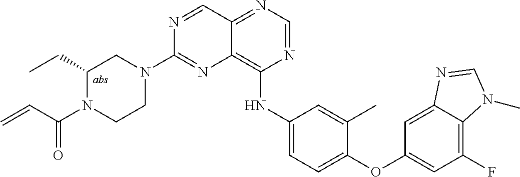 [SMILES: C=CC(=O)N1CCN(c2ncc3ncnc(Nc4ccc(Oc5cc(F)c6c(c5)ncn6C)c(C)c4)c3n2)C[C@H]1CC] |
| 164 | 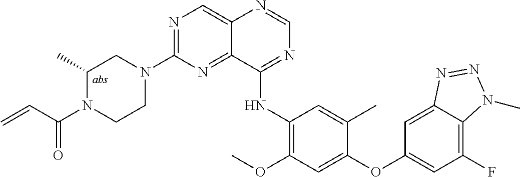 [SMILES: C=CC(=O)N1CCN(c2ncc3ncnc(Nc4cc(C)c(Oc5cc(F)c6c(c5)nnn6C)cc4OC)c3n2)C[C@H]1C] |
| 165 | 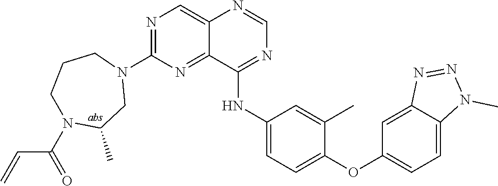 [SMILES: C=CC(=O)N1CCCN(c2ncc3ncnc(Nc4ccc(Oc5ccc6c(c5)nnn6C)c(C)c4)c3n2)C[C@@H]1C] |
| 166 | 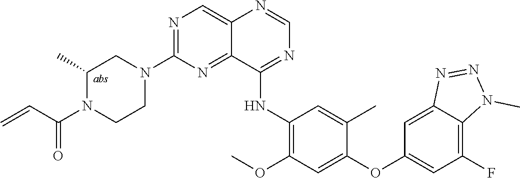 [SMILES: C=CC(=O)N1CCN(c2ncc3ncnc(Nc4cc(C)c(Oc5cc(F)c6c(c5)nnn6C)cc4OC)c3n2)C[C@H]1C] |
| 167 | 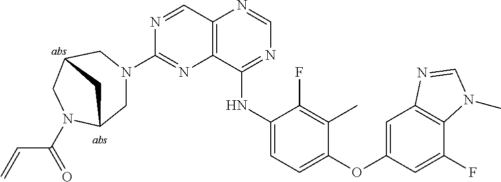 [SMILES: C=CC(=O)N1C[C@H]2C[C@@H]1CN(c1ncc3ncnc(Nc4ccc(Oc5cc(F)c6c(c5)ncn6C)c(C)c4F)c3n1)C2] |
| 168 | 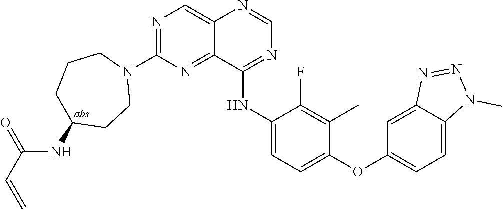 [SMILES: C=CC(=O)N[C@H]1CCCN(c2ncc3ncnc(Nc4ccc(Oc5ccc6c(c5)nnn6C)c(C)c4F)c3n2)CC1] |
| 169 | 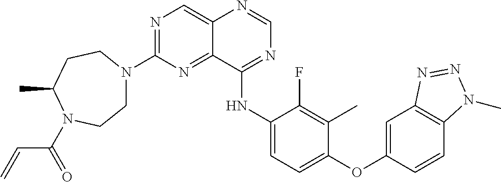 [SMILES: C=CC(=O)N1CCN(c2ncc3ncnc(Nc4ccc(Oc5ccc6c(c5)nnn6C)c(C)c4F)c3n2)CC[C@@H]1C] |
| 170 | 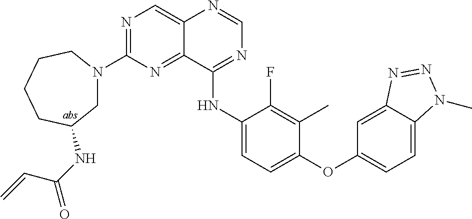 [SMILES: C=CC(=O)N[C@@H]1CCCCN(c2ncc3ncnc(Nc4ccc(Oc5ccc6c(c5)nnn6C)c(C)c4F)c3n2)C1] |
| 171 | 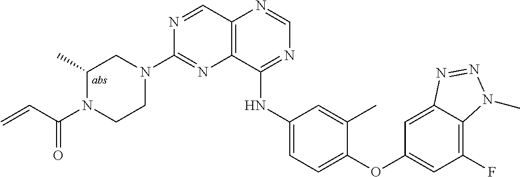 [SMILES: C=CC(=O)N1CCN(c2ncc3ncnc(Nc4ccc(Oc5cc(F)c6c(c5)nnn6C)c(C)c4)c3n2)C[C@H]1C] |
| 172 | 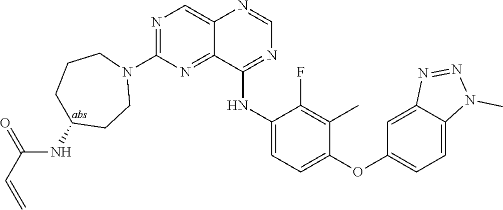 [SMILES: C=CC(=O)N[C@@H]1CCCN(c2ncc3ncnc(Nc4ccc(Oc5ccc6c(c5)nnn6C)c(C)c4F)c3n2)CC1] |
| 173 | 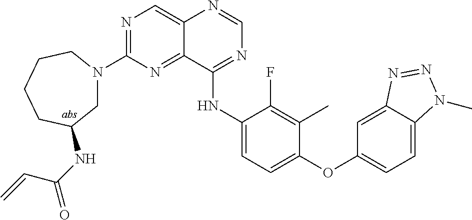 [SMILES: C=CC(=O)N[C@H]1CCCCN(c2ncc3ncnc(Nc4ccc(Oc5ccc6c(c5)nnn6C)c(C)c4F)c3n2)C1] |
| 174 | 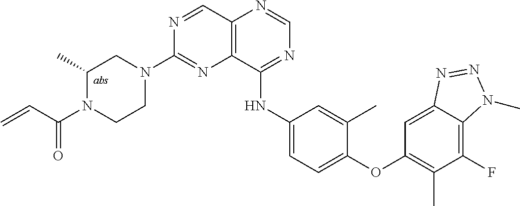 [SMILES: C=CC(=O)N1CCN(c2ncc3ncnc(Nc4ccc(Oc5cc6nnn(C)c6c(F)c5C)c(C)c4)c3n2)C[C@H]1C] |
| 175 | 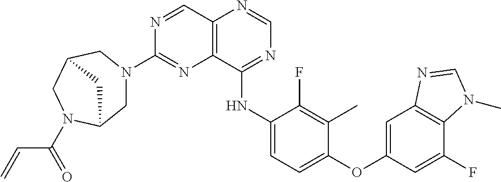 [SMILES: C=CC(=O)N1C[C@@H]2C[C@H]1CN(c1ncc3ncnc(Nc4ccc(Oc5cc(F)c6c(c5)ncn6C)c(C)c4F)c3n1)C2] |
| 176 | 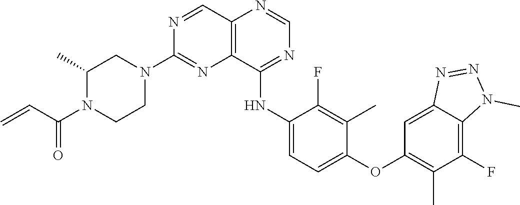 [SMILES: C=CC(=O)N1CCN(c2ncc3ncnc(Nc4ccc(Oc5cc6nnn(C)c6c(F)c5C)c(C)c4F)c3n2)C[C@H]1C] |
| 177 | 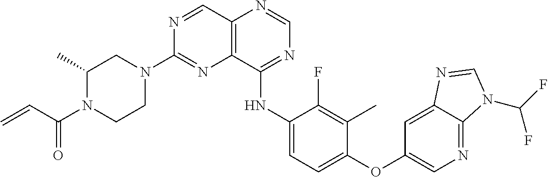 [SMILES: C=CC(=O)N1CCN(c2ncc3ncnc(Nc4ccc(Oc5cnc6c(c5)ncn6C(F)F)c(C)c4F)c3n2)C[C@H]1C] |
| 178 | 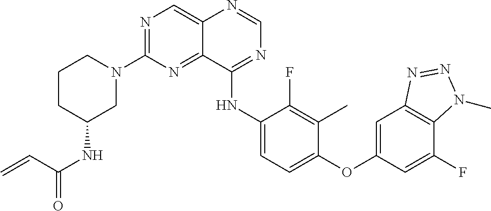 [SMILES: C=CC(=O)N[C@@H]1CCCN(c2ncc3ncnc(Nc4ccc(Oc5cc(F)c6c(c5)nnn6C)c(C)c4F)c3n2)C1] |
| 179 | 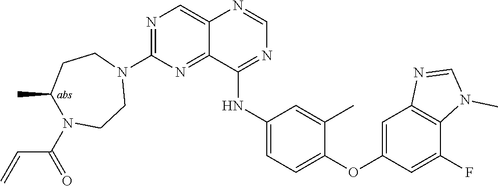 [SMILES: C=CC(=O)N1CCN(c2ncc3ncnc(Nc4ccc(Oc5cc(F)c6c(c5)ncn6C)c(C)c4)c3n2)CC[C@@H]1C] |
| 180 | 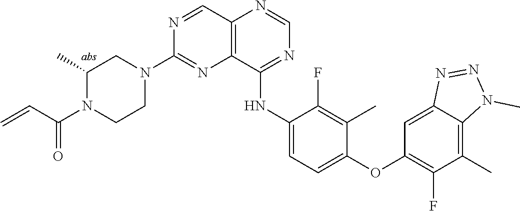 [SMILES: C=CC(=O)N1CCN(c2ncc3ncnc(Nc4ccc(Oc5cc6nnn(C)c6c(C)c5F)c(C)c4F)c3n2)C[C@H]1C] |
| 181 | 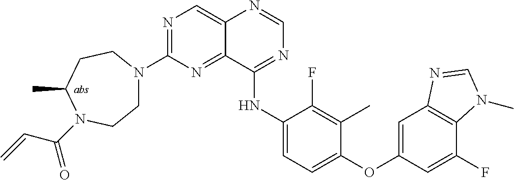 [SMILES: C=CC(=O)N1CCN(c2ncc3ncnc(Nc4ccc(Oc5cc(F)c6c(c5)ncn6C)c(C)c4F)c3n2)CC[C@@H]1C] |
| 182 | 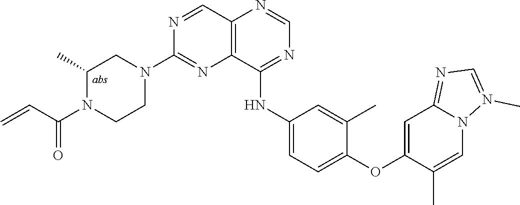 |
| 183 | 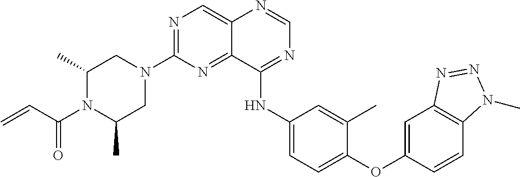 [SMILES: C=CC(=O)N1[C@H](C)CN(c2ncc3ncnc(Nc4ccc(Oc5ccc6c(c5)nnn6C)c(C)c4)c3n2)C[C@H]1C] |
| 184 | 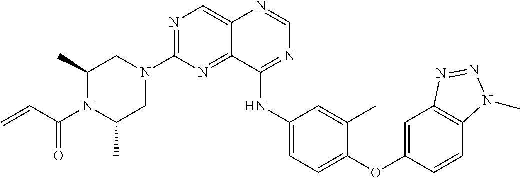 [SMILES: C=CC(=O)N1[C@@H](C)CN(c2ncc3ncnc(Nc4ccc(Oc5ccc6c(c5)nnn6C)c(C)c4)c3n2)C[C@@H]1C] |
| 185 | 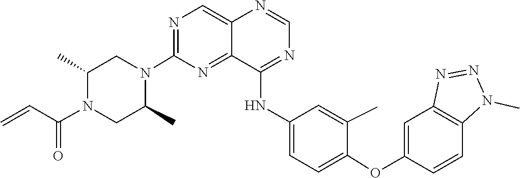 [SMILES: C=CC(=O)N1C[C@H](C)N(c2ncc3ncnc(Nc4ccc(Oc5ccc6c(c5)nnn6C)c(C)c4)c3n2)C[C@H]1C] |
| 186 | 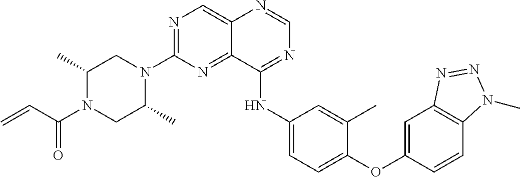 [SMILES: C=CC(=O)N1C[C@@H](C)N(c2ncc3ncnc(Nc4ccc(Oc5ccc6c(c5)nnn6C)c(C)c4)c3n2)C[C@H]1C] |
| 187 | 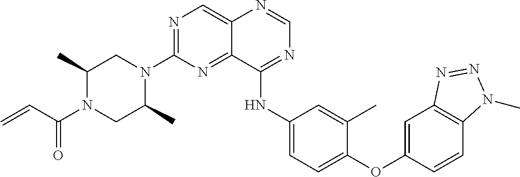 [SMILES: C=CC(=O)N1C[C@H](C)N(c2ncc3ncnc(Nc4ccc(Oc5ccc6c(c5)nnn6C)c(C)c4)c3n2)C[C@@H]1C] |
| 188 | 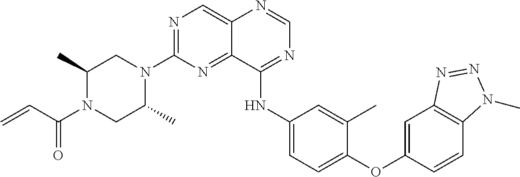 [SMILES: C=CC(=O)N1C[C@@H](C)N(c2ncc3ncnc(Nc4ccc(Oc5ccc6c(c5)nnn6C)c(C)c4)c3n2)C[C@@H]1C] |
| 189 | 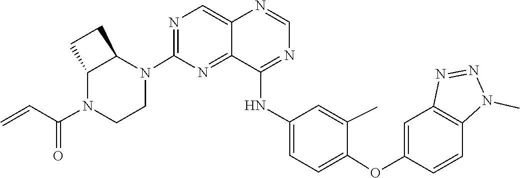 [SMILES: C=CC(=O)N1CCN(c2ncc3ncnc(Nc4ccc(Oc5ccc6c(c5)nnn6C)c(C)c4)c3n2)[C@@H]2CC[C@H]21] |
| 190 | 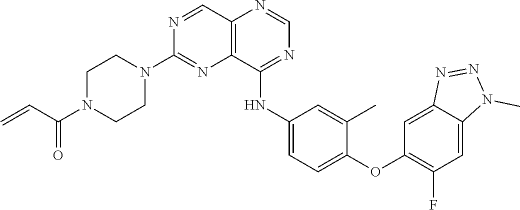 [SMILES: C=CC(=O)N1CCN(c2ncc3ncnc(Nc4ccc(Oc5cc6nnn(C)c6cc5F)c(C)c4)c3n2)CC1] |
| 191 | 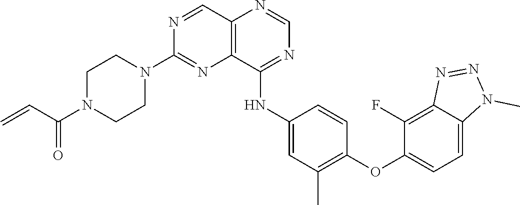 [SMILES: C=CC(=O)N1CCN(c2ncc3ncnc(Nc4ccc(Oc5ccc6c(nnn6C)c5F)c(C)c4)c3n2)CC1] |
| 192 | 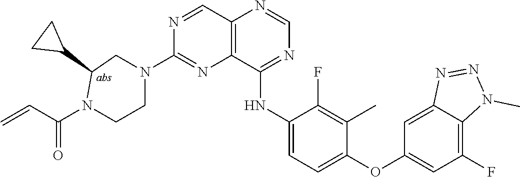 [SMILES: C=CC(=O)N1CCN(c2ncc3ncnc(Nc4ccc(Oc5cc(F)c6c(c5)nnn6C)c(C)c4F)c3n2)C[C@@H]1C1CC1] |
| 193 | 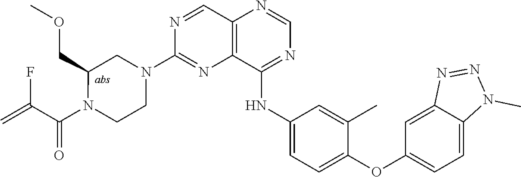 [SMILES: C=C(F)C(=O)N1CCN(c2ncc3ncnc(Nc4ccc(Oc5ccc6c(c5)nnn6C)c(C)c4)c3n2)C[C@@H]1COC] |
| 194 | 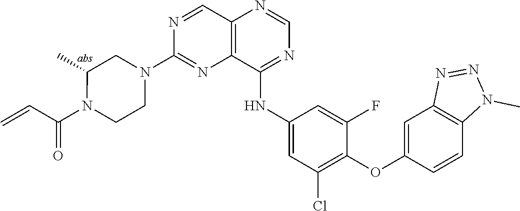 [SMILES: C=CC(=O)N1CCN(c2ncc3ncnc(Nc4cc(F)c(Oc5ccc6c(c5)nnn6C)c(Cl)c4)c3n2)C[C@H]1C] |
| 195 | 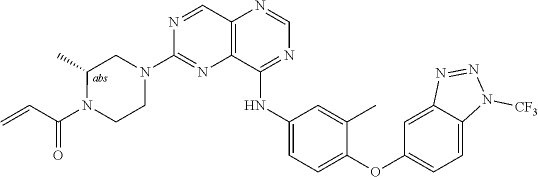 [SMILES: C=CC(=O)N1CCN(c2ncc3ncnc(Nc4ccc(Oc5ccc6c(c5)nnn6C(F)(F)F)c(C)c4)c3n2)C[C@H]1C] |
| 196 | 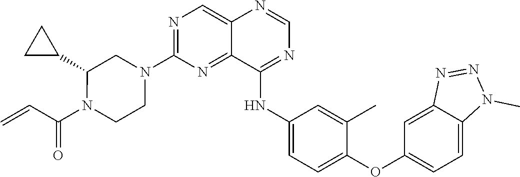 [SMILES: C=CC(=O)N1CCN(c2ncc3ncnc(Nc4ccc(Oc5ccc6c(c5)nnn6C)c(C)c4)c3n2)C[C@H]1C1CC1] |
| 197 | 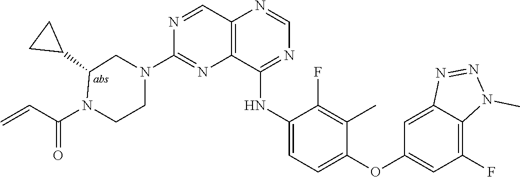 [SMILES: C=CC(=O)N1CCN(c2ncc3ncnc(Nc4ccc(Oc5cc(F)c6c(c5)nnn6C)c(C)c4F)c3n2)C[C@H]1C1CC1] |
| 198 | 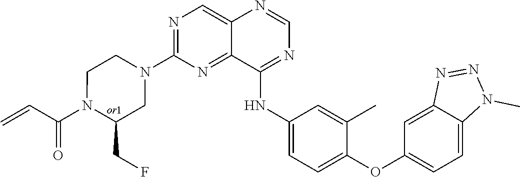 [SMILES: C=CC(=O)N1CCN(c2ncc3ncnc(Nc4ccc(Oc5ccc6c(c5)nnn6C)c(C)c4)c3n2)C[C@H]1CF] |
| 199 | 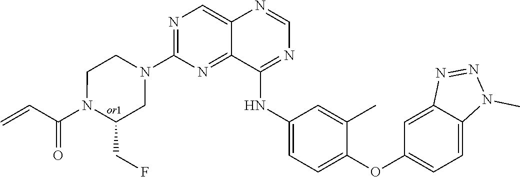 [SMILES: C=CC(=O)N1CCN(c2ncc3ncnc(Nc4ccc(Oc5ccc6c(c5)nnn6C)c(C)c4)c3n2)C[C@@H]1CF] |
| 200 | 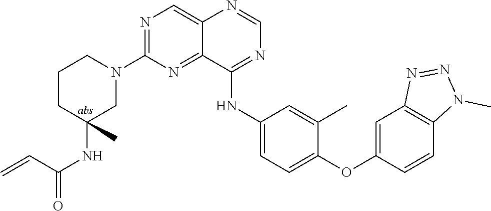 [SMILES: C=CC(=O)N[C@]1(C)CCCN(c2ncc3ncnc(Nc4ccc(Oc5ccc6c(c5)nnn6C)c(C)c4)c3n2)C1] |
| 201 | 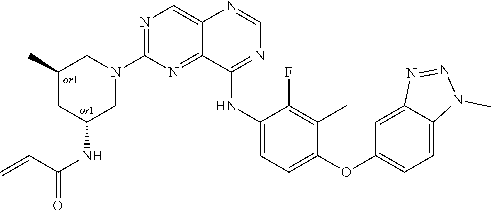 [SMILES: C=CC(=O)N[C@@H]1C[C@@H](C)CN(c2ncc3ncnc(Nc4ccc(Oc5ccc6c(c5)nnn6C)c(C)c4F)c3n2)C1] |
| 202 | 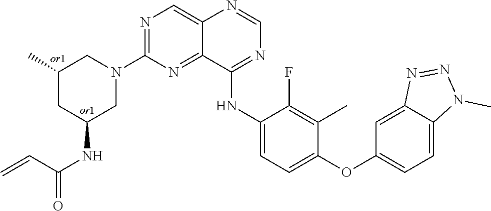 [SMILES: C=CC(=O)N[C@H]1C[C@H](C)CN(c2ncc3ncnc(Nc4ccc(Oc5ccc6c(c5)nnn6C)c(C)c4F)c3n2)C1] |
| 203 | 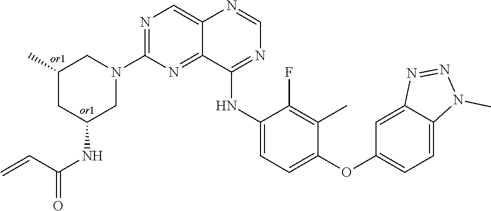 [SMILES: C=CC(=O)N[C@@H]1C[C@H](C)CN(c2ncc3ncnc(Nc4ccc(Oc5ccc6c(c5)nnn6C)c(C)c4F)c3n2)C1] |
| 204 | 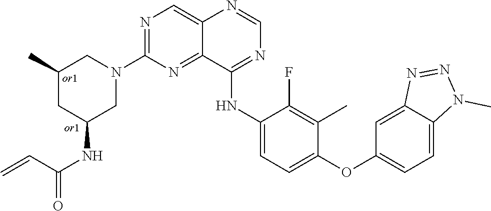 [SMILES: C=CC(=O)N[C@H]1C[C@@H](C)CN(c2ncc3ncnc(Nc4ccc(Oc5ccc6c(c5)nnn6C)c(C)c4F)c3n2)C1] |
| 205 | 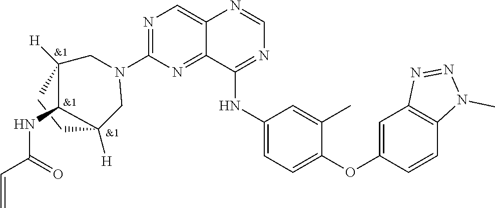 [SMILES: C=CC(=O)N[C@@H]1[C@@H](CC)CN(c2ncc3ncnc(Nc4ccc(Oc5ccc6c(c5)nnn6C)c(C)c4)c3n2)C[C@H]1C] |
| 206 | 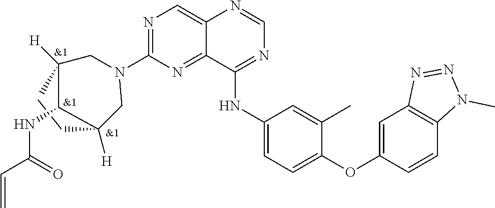 [SMILES: C=CC(=O)N[C@H]1[C@@H](CC)CN(c2ncc3ncnc(Nc4ccc(Oc5ccc6c(c5)nnn6C)c(C)c4)c3n2)C[C@H]1C] |
| 207 | 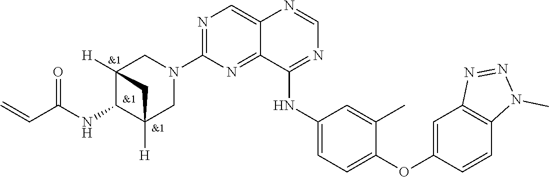 [SMILES: C=CC(=O)N[C@H]1[C@@H]2C[C@H]1CN(c1ncc3ncnc(Nc4ccc(Oc5ccc6c(c5)nnn6C)c(C)c4)c3n1)C2] |
| 208 | 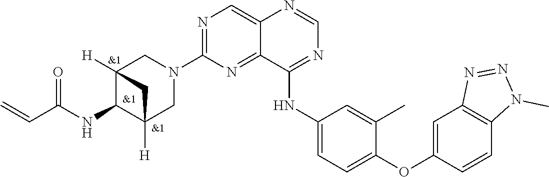 [SMILES: C=CC(=O)N[C@@H]1[C@@H]2C[C@H]1CN(c1ncc3ncnc(Nc4ccc(Oc5ccc6c(c5)nnn6C)c(C)c4)c3n1)C2] |
| 209 | 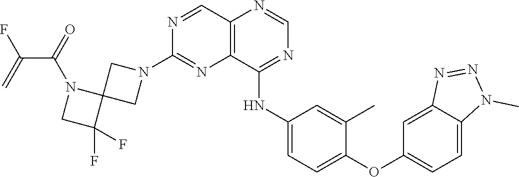 [SMILES: C=C(F)C(=O)N1CC(F)(F)C12CN(c1ncc3ncnc(Nc4ccc(Oc5ccc6c(c5)nnn6C)c(C)c4)c3n1)C2] |
| 210 | 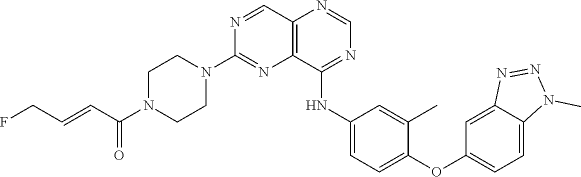 [SMILES: Cc1cc(Nc2ncnc3cnc(N4CCN(C(=O)/C=C/CF)CC4)nc23)ccc1Oc1ccc2c(c1)nnn2C] |
| 211 | 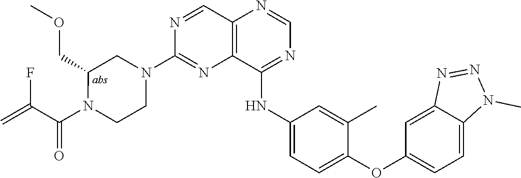 [SMILES: C=C(F)C(=O)N1CCN(c2ncc3ncnc(Nc4ccc(Oc5ccc6c(c5)nnn6C)c(C)c4)c3n2)C[C@H]1COC] |
| 212 | 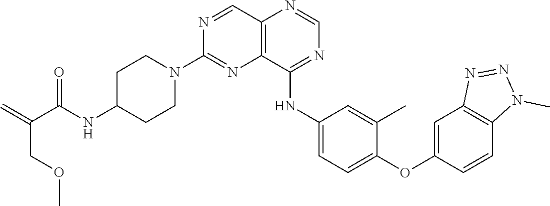 [SMILES: C=C(COC)C(=O)NC1CCN(c2ncc3ncnc(Nc4ccc(Oc5ccc6c(c5)nnn6C)c(C)c4)c3n2)CC1] |
| 214 | 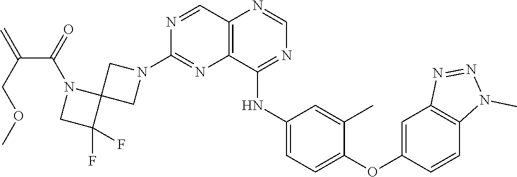 [SMILES: C=C(COC)C(=O)N1CC(F)(F)C12CN(c1ncc3ncnc(Nc4ccc(Oc5ccc6c(c5)nnn6C)c(C)c4)c3n1)C2] |
| 215 | 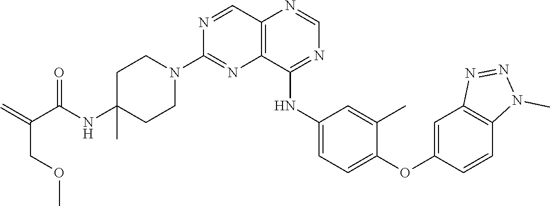 [SMILES: C=C(COC)C(=O)NC1(C)CCN(c2ncc3ncnc(Nc4ccc(Oc5ccc6c(c5)nnn6C)c(C)c4)c3n2)CC1] |
| 216 | 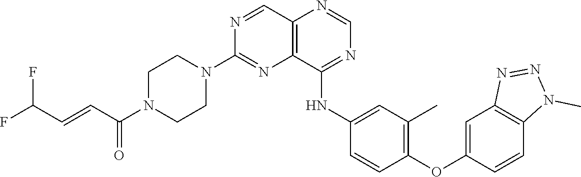 [SMILES: Cc1cc(Nc2ncnc3cnc(N4CCN(C(=O)/C=C/C(F)F)CC4)nc23)ccc1Oc1ccc2c(c1)nnn2C] |
| 217 | 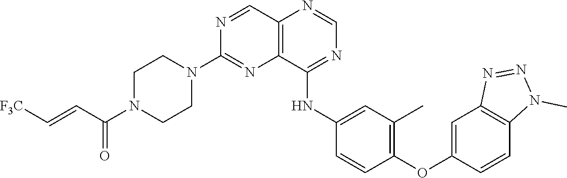 [SMILES: Cc1cc(Nc2ncnc3cnc(N4CCN(C(=O)/C=C/C(F)(F)F)CC4)nc23)ccc1Oc1ccc2c(c1)nnn2C] |
| 218 | 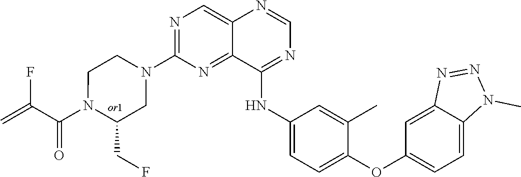 [SMILES: C=C(F)C(=O)N1CCN(c2ncc3ncnc(Nc4ccc(Oc5ccc6c(c5)nnn6C)c(C)c4)c3n2)C[C@@H]1CF] |
| 219 | 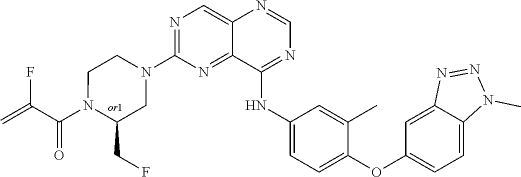 [SMILES: C=C(F)C(=O)N1CCN(c2ncc3ncnc(Nc4ccc(Oc5ccc6c(c5)nnn6C)c(C)c4)c3n2)C[C@H]1CF] |
| 220 | 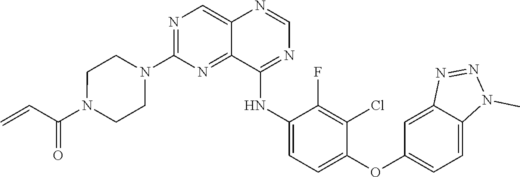 [SMILES: C=CC(=O)N1CCN(c2ncc3ncnc(Nc4ccc(Oc5ccc6c(c5)nnn6C)c(Cl)c4F)c3n2)CC1] |
| 221 | 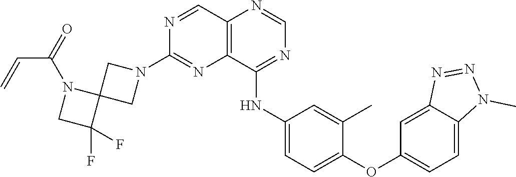 [SMILES: C=CC(=O)N1CC(F)(F)C12CN(c1ncc3ncnc(Nc4ccc(Oc5ccc6c(c5)nnn6C)c(C)c4)c3n1)C2] |
| 222 | 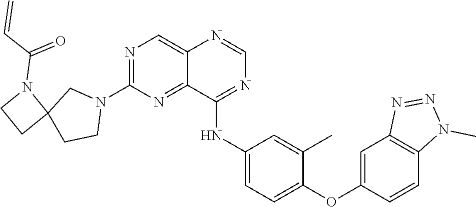 [SMILES: C=CC(=O)N1CCC12CCN(c1ncc3ncnc(Nc4ccc(Oc5ccc6c(c5)nnn6C)c(C)c4)c3n1)C2] |
| 223 | 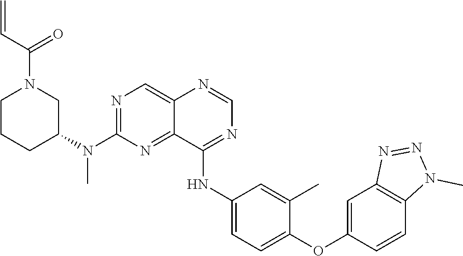 [SMILES: C=CC(=O)N1CCC[C@@H](N(C)c2ncc3ncnc(Nc4ccc(Oc5ccc6c(c5)nnn6C)c(C)c4)c3n2)C1] |
| 224 | 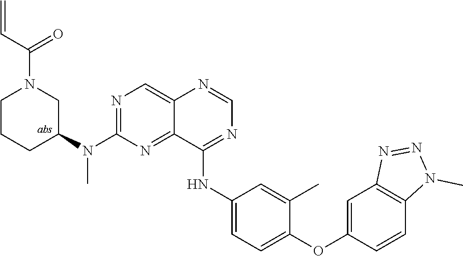 [SMILES: C=CC(=O)N1CCC[C@H](N(C)c2ncc3ncnc(Nc4ccc(Oc5ccc6c(c5)nnn6C)c(C)c4)c3n2)C1] |
| 225 | 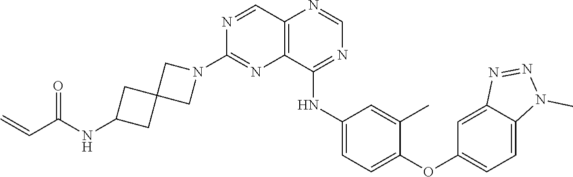 [SMILES: C=CC(=O)NC1CC2(C1)CN(c1ncc3ncnc(Nc4ccc(Oc5ccc6c(c5)nnn6C)c(C)c4)c3n1)C2] |
| 226 | 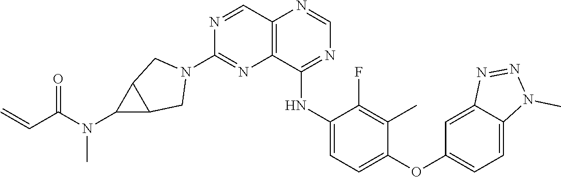 [SMILES: C=CC(=O)N(C)C1C2CN(c3ncc4ncnc(Nc5ccc(Oc6ccc7c(c6)nnn7C)c(C)c5F)c4n3)CC21] |
| 227 | 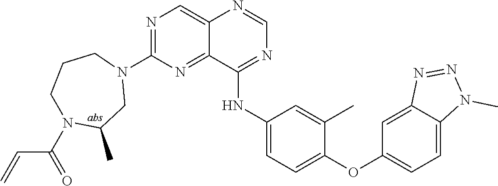 [SMILES: C=CC(=O)N1CCCN(c2ncc3ncnc(Nc4ccc(Oc5ccc6c(c5)nnn6C)c(C)c4)c3n2)C[C@H]1C] |
| 228 | 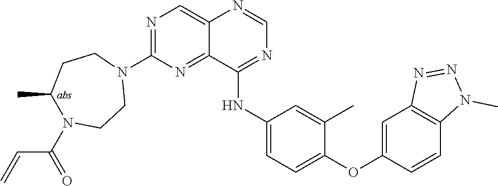 [SMILES: C=CC(=O)N1CCN(c2ncc3ncnc(Nc4ccc(Oc5ccc6c(c5)nnn6C)c(C)c4)c3n2)CC[C@@H]1C] |
| 229 | 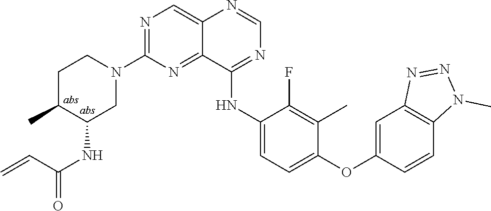 [SMILES: C=CC(=O)N[C@H]1CN(c2ncc3ncnc(Nc4ccc(Oc5ccc6c(c5)nnn6C)c(C)c4F)c3n2)CC[C@@H]1C] |
| 230 | 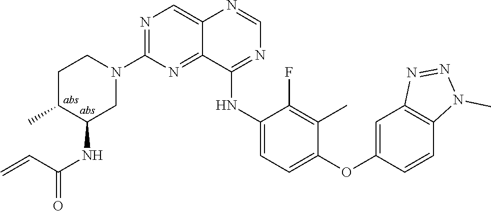 [SMILES: C=CC(=O)N[C@@H]1CN(c2ncc3ncnc(Nc4ccc(Oc5ccc6c(c5)nnn6C)c(C)c4F)c3n2)CC[C@H]1C] |
| 231 | 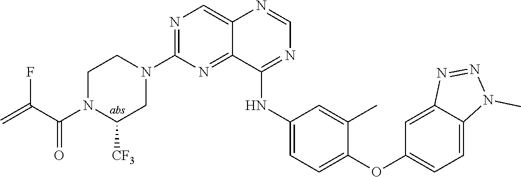 [SMILES: C=C(F)C(=O)N1CCN(c2ncc3ncnc(Nc4ccc(Oc5ccc6c(c5)nnn6C)c(C)c4)c3n2)C[C@@H]1C(F)(F)F] |
| 232 | 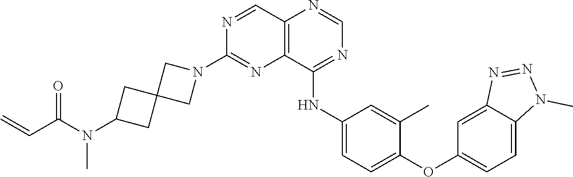 [SMILES: C=CC(=O)N(C)C1CC2(C1)CN(c1ncc3ncnc(Nc4ccc(Oc5ccc6c(c5)nnn6C)c(C)c4)c3n1)C2] |
| 233 | 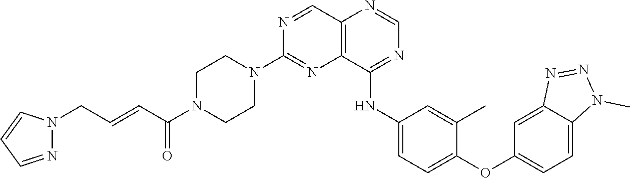 [SMILES: Cc1cc(Nc2ncnc3cnc(N4CCN(C(=O)/C=C/Cn5cccn5)CC4)nc23)ccc1Oc1ccc2c(c1)nnn2C] |
| 234 | 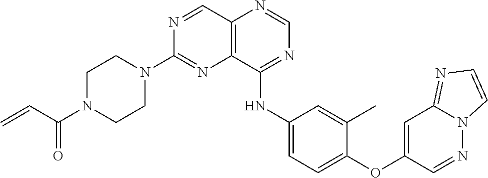 [SMILES: C=CC(=O)N1CCN(c2ncc3ncnc(Nc4ccc(Oc5cnn6ccnc6c5)c(C)c4)c3n2)CC1] |
| 235 | 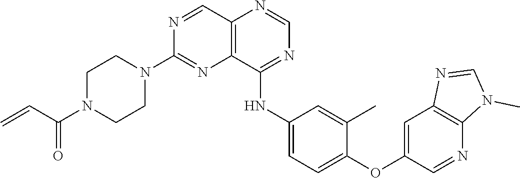 [SMILES: C=CC(=O)N1CCN(c2ncc3ncnc(Nc4ccc(Oc5cnc6c(c5)ncn6C)c(C)c4)c3n2)CC1] |
| 236 | 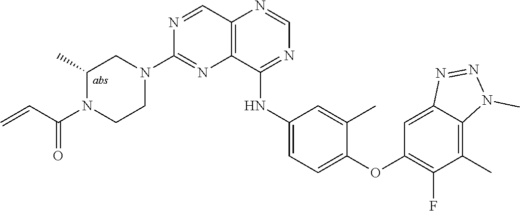 [SMILES: C=CC(=O)N1CCN(c2ncc3ncnc(Nc4ccc(Oc5cc6nnn(C)c6c(C)c5F)c(C)c4)c3n2)C[C@H]1C] |
| 237 | 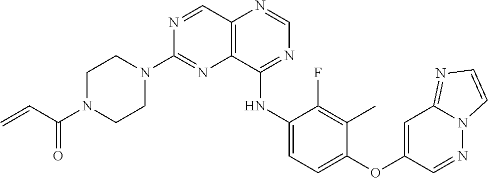 [SMILES: C=CC(=O)N1CCN(c2ncc3ncnc(Nc4ccc(Oc5cnn6ccnc6c5)c(C)c4F)c3n2)CC1] |
| 238 | 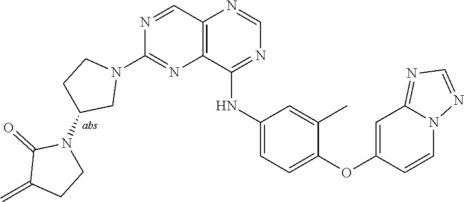 [SMILES: C=C1CCN([C@@H]2CCN(c3ncc4ncnc(Nc5ccc(Oc6ccn7ncnc7c6)c(C)c5)c4n3)C2)C1=O] |
| 239 | 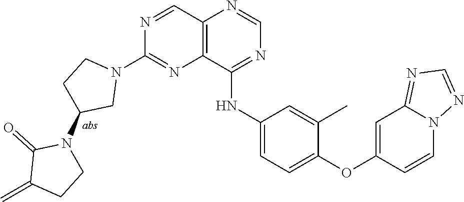 [SMILES: C=C1CCN([C@H]2CCN(c3ncc4ncnc(Nc5ccc(Oc6ccn7ncnc7c6)c(C)c5)c4n3)C2)C1=O] |
| 240 | 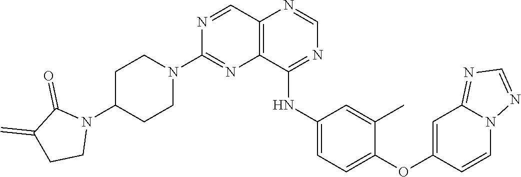 [SMILES: C=C1CCN(C2CCN(c3ncc4ncnc(Nc5ccc(Oc6ccn7ncnc7c6)c(C)c5)c4n3)CC2)C1=O] |
| 241 | 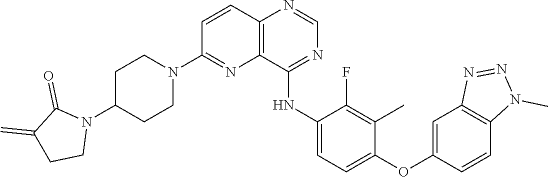 |
| 242 | 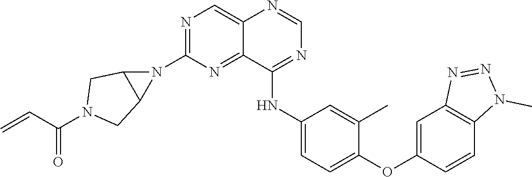 [SMILES: C=CC(=O)N1CC2C(C1)N2c1ncc2ncnc(Nc3ccc(Oc4ccc5c(c4)nnn5C)c(C)c3)c2n1] |
| 243 | 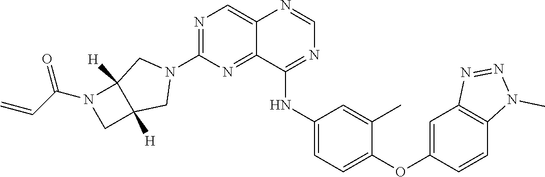 [SMILES: C=CC(=O)N1C[C@H]2CN(c3ncc4ncnc(Nc5ccc(Oc6ccc7c(c6)nnn7C)c(C)c5)c4n3)C[C@H]21] |
| 244 | 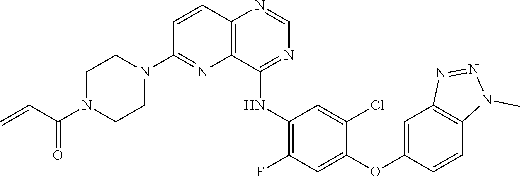 [SMILES: C=CC(=O)N1CCN(c2ccc3ncnc(Nc4cc(Cl)c(Oc5ccc6c(c5)nnn6C)cc4F)c3n2)CC1] |
| 245 | 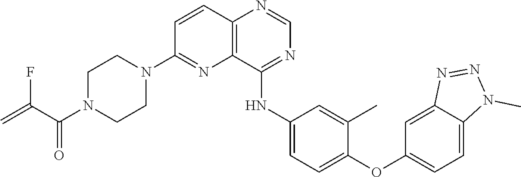 [SMILES: C=C(F)C(=O)N1CCN(c2ccc3ncnc(Nc4ccc(Oc5ccc6c(c5)nnn6C)c(C)c4)c3n2)CC1] |
| 246 | 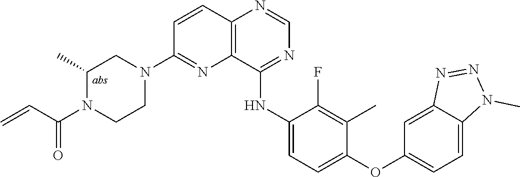 [SMILES: C=CC(=O)N1CCN(c2ccc3ncnc(Nc4ccc(Oc5ccc6c(c5)nnn6C)c(C)c4F)c3n2)C[C@H]1C] |
| 247 | 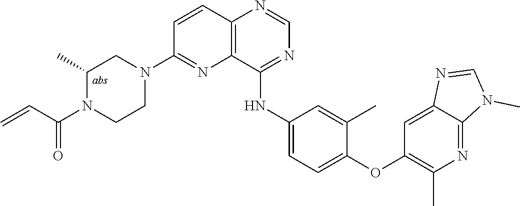 [SMILES: C=CC(=O)N1CCN(c2ccc3ncnc(Nc4ccc(Oc5cc6ncn(C)c6nc5C)c(C)c4)c3n2)C[C@H]1C] |
| 248 | 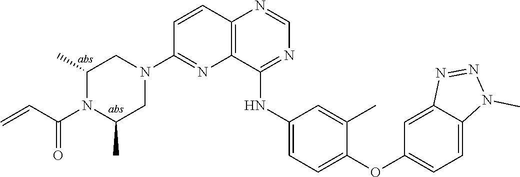 [SMILES: C=CC(=O)N1[C@H](C)CN(c2ccc3ncnc(Nc4ccc(Oc5ccc6c(c5)nnn6C)c(C)c4)c3n2)C[C@H]1C] |
| 249 | 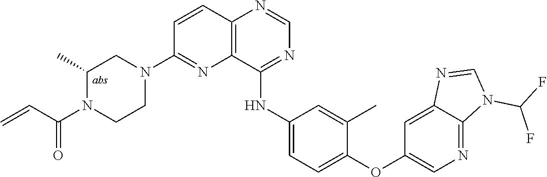 [SMILES: C=CC(=O)N1CCN(c2ccc3ncnc(Nc4ccc(Oc5cnc6c(c5)ncn6C(F)F)c(C)c4)c3n2)C[C@H]1C] |
| 250 | 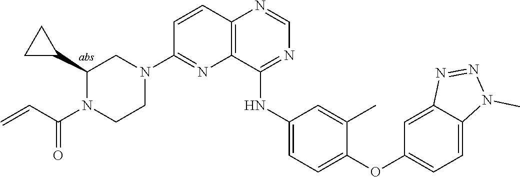 [SMILES: C=CC(=O)N1CCN(c2ccc3ncnc(Nc4ccc(Oc5ccc6c(c5)nnn6C)c(C)c4)c3n2)C[C@@H]1C1CC1] |
| 251 | 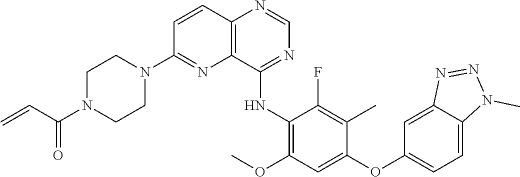 [SMILES: C=CC(=O)N1CCN(c2ccc3ncnc(Nc4c(OC)cc(Oc5ccc6c(c5)nnn6C)c(C)c4F)c3n2)CC1] |
| 252 | 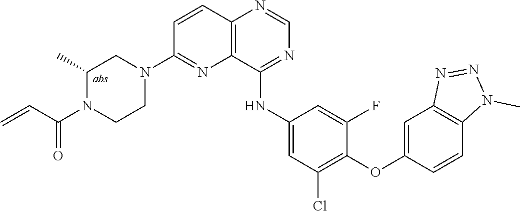 [SMILES: C=CC(=O)N1CCN(c2ccc3ncnc(Nc4cc(F)c(Oc5ccc6c(c5)nnn6C)c(Cl)c4)c3n2)C[C@H]1C] |
| 253 | 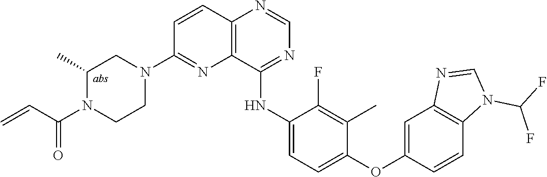 [SMILES: C=CC(=O)N1CCN(c2ccc3ncnc(Nc4ccc(Oc5ccc6c(c5)ncn6C(F)F)c(C)c4F)c3n2)C[C@H]1C] |
| 254 | 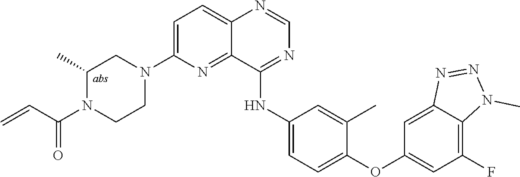 [SMILES: C=CC(=O)N1CCN(c2ccc3ncnc(Nc4ccc(Oc5cc(F)c6c(c5)nnn6C)c(C)c4)c3n2)C[C@H]1C] |
| 255 | 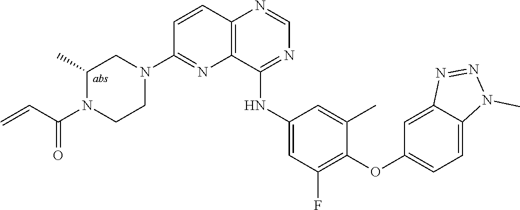 [SMILES: C=CC(=O)N1CCN(c2ccc3ncnc(Nc4cc(C)c(Oc5ccc6c(c5)nnn6C)c(F)c4)c3n2)C[C@H]1C] |
| 256 | 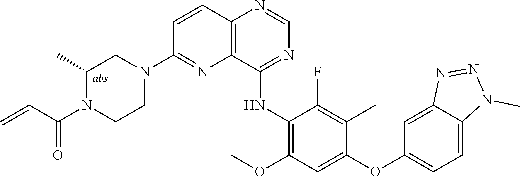 [SMILES: C=CC(=O)N1CCN(c2ccc3ncnc(Nc4c(OC)cc(Oc5ccc6c(c5)nnn6C)c(C)c4F)c3n2)C[C@H]1C] |
| 257 | 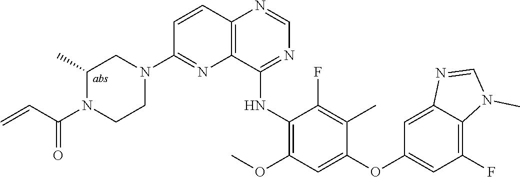 [SMILES: C=CC(=O)N1CCN(c2ccc3ncnc(Nc4c(OC)cc(Oc5cc(F)c6c(c5)ncn6C)c(C)c4F)c3n2)C[C@H]1C] |
| 258 | 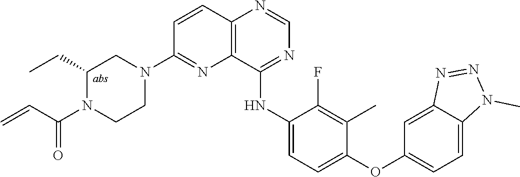 [SMILES: C=CC(=O)N1CCN(c2ccc3ncnc(Nc4ccc(Oc5ccc6c(c5)nnn6C)c(C)c4F)c3n2)C[C@H]1CC] |
| 259 | 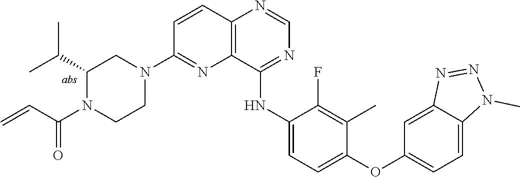 [SMILES: C=CC(=O)N1CCN(c2ccc3ncnc(Nc4ccc(Oc5ccc6c(c5)nnn6C)c(C)c4F)c3n2)C[C@H]1C(C)C] |
| 260 | 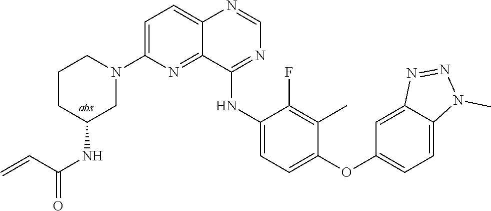 [SMILES: C=CC(=O)N[C@@H]1CCCN(c2ccc3ncnc(Nc4ccc(Oc5ccc6c(c5)nnn6C)c(C)c4F)c3n2)C1] |
| 261 | 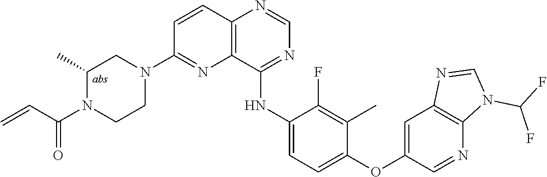 [SMILES: C=CC(=O)N1CCN(c2ccc3ncnc(Nc4ccc(Oc5cnc6c(c5)ncn6C(F)F)c(C)c4F)c3n2)C[C@H]1C] |
| 262 | 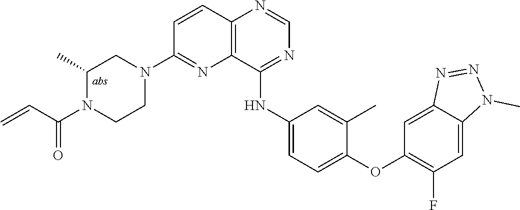 [SMILES: C=CC(=O)N1CCN(c2ccc3ncnc(Nc4ccc(Oc5cc6nnn(C)c6cc5F)c(C)c4)c3n2)C[C@H]1C] |
| 263 | 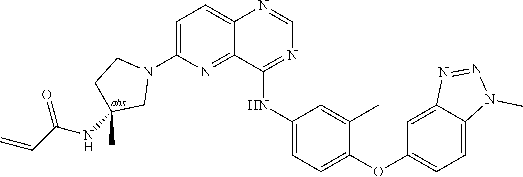 [SMILES: C=CC(=O)N[C@]1(C)CCN(c2ccc3ncnc(Nc4ccc(Oc5ccc6c(c5)nnn6C)c(C)c4)c3n2)C1] |
| 264 | 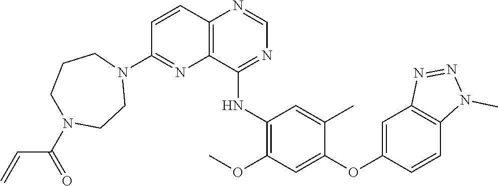 [SMILES: C=CC(=O)N1CCCN(c2ccc3ncnc(Nc4cc(C)c(Oc5ccc6c(c5)nnn6C)cc4OC)c3n2)CC1] |
| 265 | 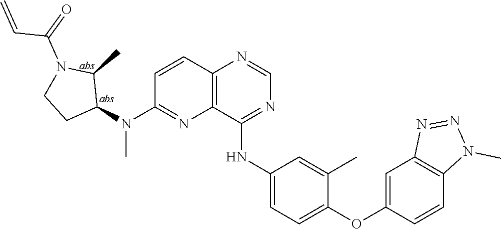 [SMILES: C=CC(=O)N1CC[C@H](N(C)c2ccc3ncnc(Nc4ccc(Oc5ccc6c(c5)nnn6C)c(C)c4)c3n2)[C@@H]1C] |
| 266 | 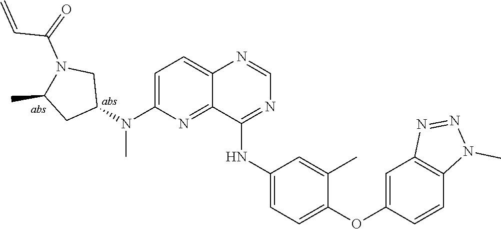 [SMILES: C=CC(=O)N1C[C@H](N(C)c2ccc3ncnc(Nc4ccc(Oc5ccc6c(c5)nnn6C)c(C)c4)c3n2)C[C@H]1C] |
| 267 | 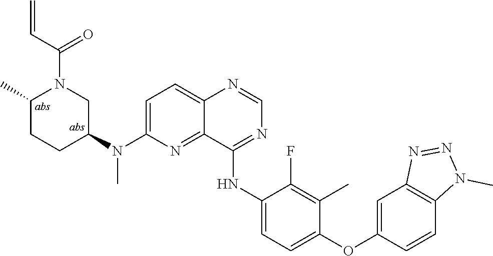 |
| 268 | 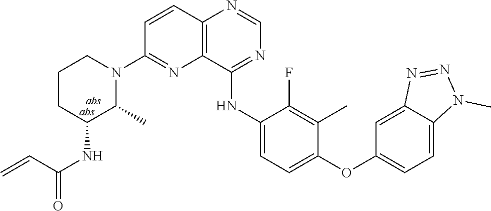 [SMILES: C=CC(=O)N[C@@H]1CCCN(c2ccc3ncnc(Nc4ccc(Oc5ccc6c(c5)nnn6C)c(C)c4F)c3n2)[C@@H]1C] |
| 269 | 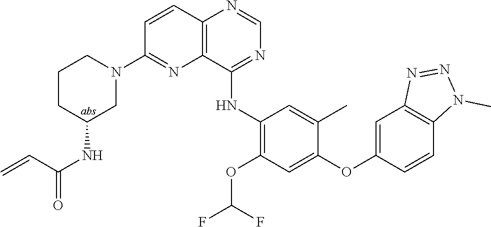 [SMILES: C=CC(=O)N[C@@H]1CCCN(c2ccc3ncnc(Nc4cc(C)c(Oc5ccc6c(c5)nnn6C)cc4OC(F)F)c3n2)C1] |
| 270 | 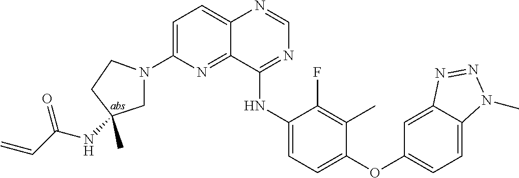 [SMILES: C=CC(=O)N[C@]1(C)CCN(c2ccc3ncnc(Nc4ccc(Oc5ccc6c(c5)nnn6C)c(C)c4F)c3n2)C1] |
| 271 | 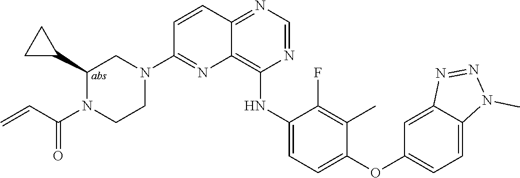 [SMILES: C=CC(=O)N1CCN(c2ccc3ncnc(Nc4ccc(Oc5ccc6c(c5)nnn6C)c(C)c4F)c3n2)C[C@@H]1C1CC1] |
| 272 | 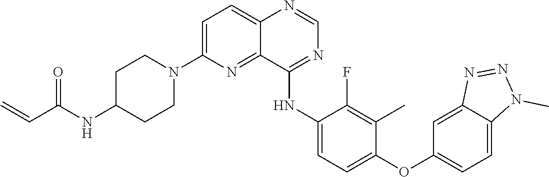 [SMILES: C=CC(=O)NC1CCN(c2ccc3ncnc(Nc4ccc(Oc5ccc6c(c5)nnn6C)c(C)c4F)c3n2)CC1] |
| 273 | 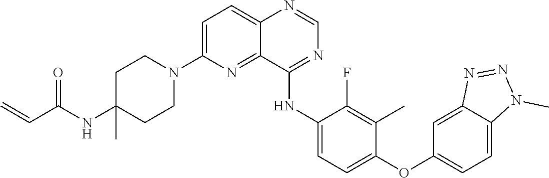 [SMILES: C=CC(=O)NC1(C)CCN(c2ccc3ncnc(Nc4ccc(Oc5ccc6c(c5)nnn6C)c(C)c4F)c3n2)CC1] |
| 274 | 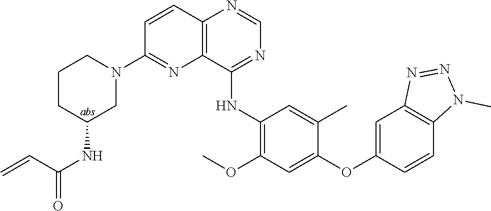 [SMILES: C=CC(=O)N[C@@H]1CCCN(c2ccc3ncnc(Nc4cc(C)c(Oc5ccc6c(c5)nnn6C)cc4OC)c3n2)C1] |
| 275 | 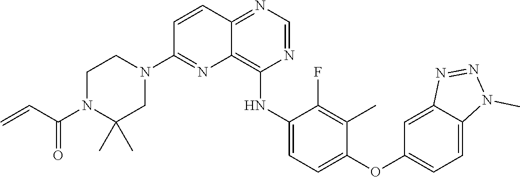 [SMILES: C=CC(=O)N1CCN(c2ccc3ncnc(Nc4ccc(Oc5ccc6c(c5)nnn6C)c(C)c4F)c3n2)CC1(C)C] |
| 276 | 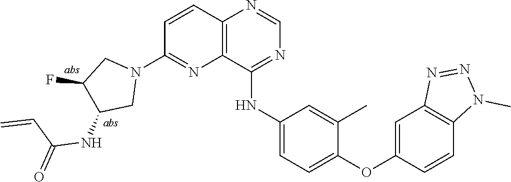 [SMILES: C=CC(=O)N[C@H]1CN(c2ccc3ncnc(Nc4ccc(Oc5ccc6c(c5)nnn6C)c(C)c4)c3n2)C[C@@H]1F] |
| 277 | 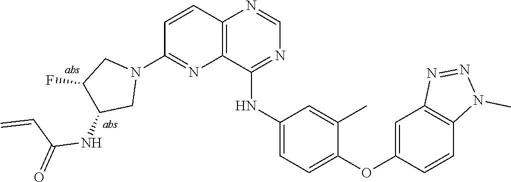 [SMILES: C=CC(=O)N[C@H]1CN(c2ccc3ncnc(Nc4ccc(Oc5ccc6c(c5)nnn6C)c(C)c4)c3n2)C[C@H]1F] |
| 278 | 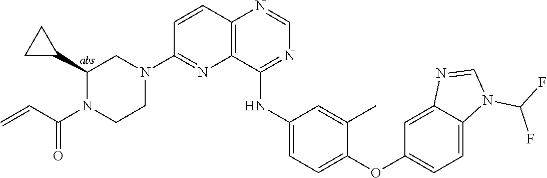 [SMILES: C=CC(=O)N1CCN(c2ccc3ncnc(Nc4ccc(Oc5ccc6c(c5)ncn6C(F)F)c(C)c4)c3n2)C[C@@H]1C1CC1] |
| 279 | 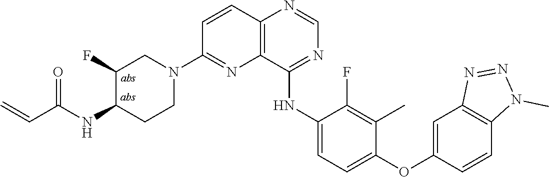 [SMILES: C=CC(=O)N[C@@H]1CCN(c2ccc3ncnc(Nc4ccc(Oc5ccc6c(c5)nnn6C)c(C)c4F)c3n2)C[C@@H]1F] |
| 280 | 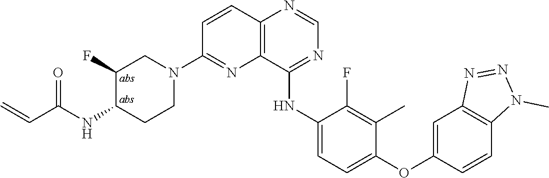 [SMILES: C=CC(=O)N[C@H]1CCN(c2ccc3ncnc(Nc4ccc(Oc5ccc6c(c5)nnn6C)c(C)c4F)c3n2)C[C@@H]1F] |
| 281 | 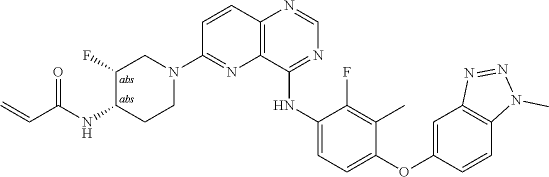 [SMILES: C=CC(=O)N[C@H]1CCN(c2ccc3ncnc(Nc4ccc(Oc5ccc6c(c5)nnn6C)c(C)c4F)c3n2)C[C@H]1F] |
| 282 | 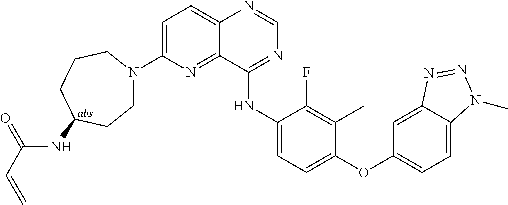 [SMILES: C=CC(=O)N[C@H]1CCCN(c2ccc3ncnc(Nc4ccc(Oc5ccc6c(c5)nnn6C)c(C)c4F)c3n2)CC1] |
| 283 | 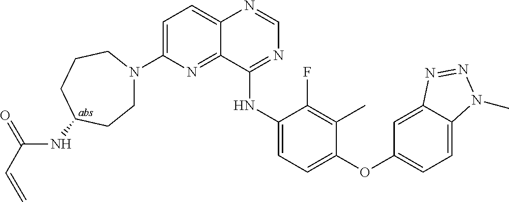 [SMILES: C=CC(=O)N[C@@H]1CCCN(c2ccc3ncnc(Nc4ccc(Oc5ccc6c(c5)nnn6C)c(C)c4F)c3n2)CC1] |
| 284 | 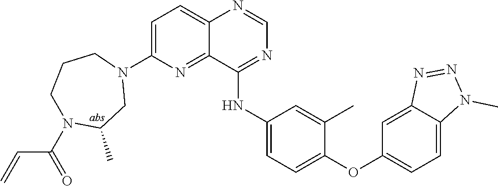 [SMILES: C=CC(=O)N1CCCN(c2ccc3ncnc(Nc4ccc(Oc5ccc6c(c5)nnn6C)c(C)c4)c3n2)C[C@@H]1C] |
| 285 | 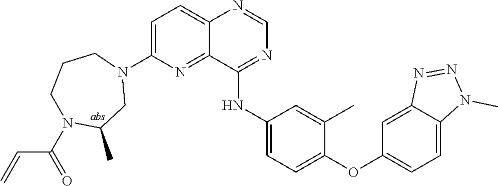 [SMILES: C=CC(=O)N1CCCN(c2ccc3ncnc(Nc4ccc(Oc5ccc6c(c5)nnn6C)c(C)c4)c3n2)C[C@H]1C] |
| 286 | 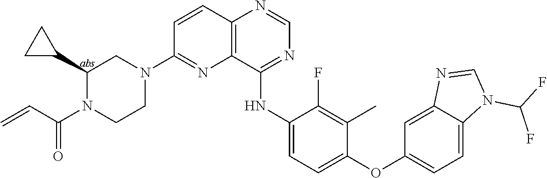 [SMILES: C=CC(=O)N1CCN(c2ccc3ncnc(Nc4ccc(Oc5ccc6c(c5)ncn6C(F)F)c(C)c4F)c3n2)C[C@@H]1C1CC1] |
| 287 | 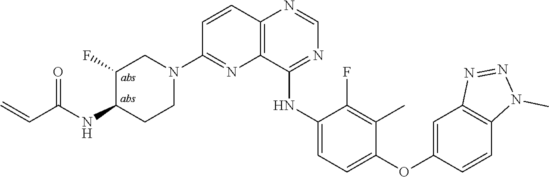 [SMILES: C=CC(=O)N[C@@H]1CCN(c2ccc3ncnc(Nc4ccc(Oc5ccc6c(c5)nnn6C)c(C)c4F)c3n2)C[C@H]1F] |
| 288 | 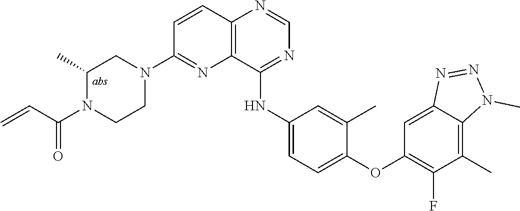 [SMILES: C=CC(=O)N1CCN(c2ccc3ncnc(Nc4ccc(Oc5cc6nnn(C)c6c(C)c5F)c(C)c4)c3n2)C[C@H]1C] |
| 289 | 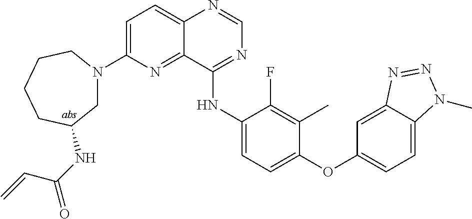 [SMILES: C=CC(=O)N[C@@H]1CCCCN(c2ccc3ncnc(Nc4ccc(Oc5ccc6c(c5)nnn6C)c(C)c4F)c3n2)C1] |
| 290 | 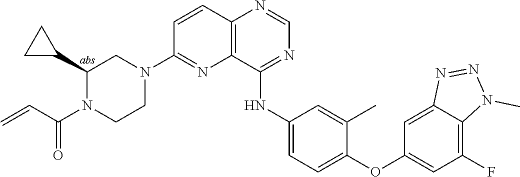 [SMILES: C=CC(=O)N1CCN(c2ccc3ncnc(Nc4ccc(Oc5cc(F)c6c(c5)nnn6C)c(C)c4)c3n2)C[C@@H]1C1CC1] |
| 291 | 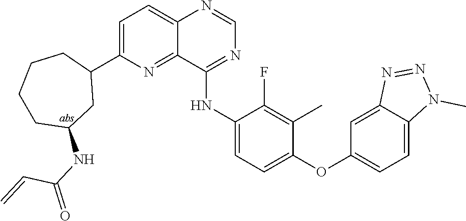 [SMILES: C=CC(=O)N[C@H]1CCCCC(c2ccc3ncnc(Nc4ccc(Oc5ccc6c(c5)nnn6C)c(C)c4F)c3n2)C1] |
| 292 | 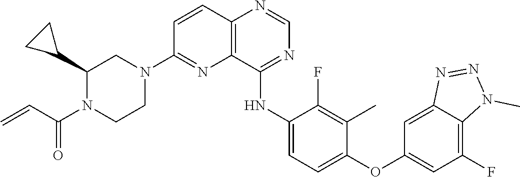 [SMILES: C=CC(=O)N1CCN(c2ccc3ncnc(Nc4ccc(Oc5cc(F)c6c(c5)nnn6C)c(C)c4F)c3n2)C[C@@H]1C1CC1] |
| 293 | 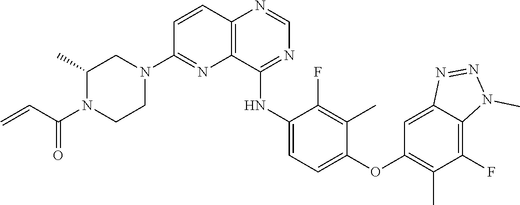 [SMILES: C=CC(=O)N1CCN(c2ccc3ncnc(Nc4ccc(Oc5cc6nnn(C)c6c(F)c5C)c(C)c4F)c3n2)C[C@H]1C] |
| 294 | 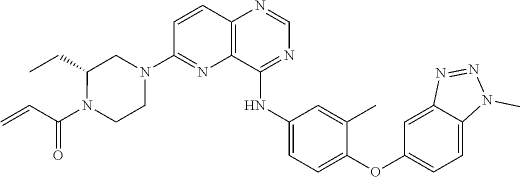 [SMILES: C=CC(=O)N1CCN(c2ccc3ncnc(Nc4ccc(Oc5ccc6c(c5)nnn6C)c(C)c4)c3n2)C[C@H]1CC] |
| 295 | 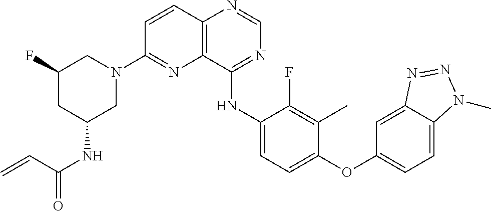 [SMILES: C=CC(=O)N[C@@H]1C[C@@H](F)CN(c2ccc3ncnc(Nc4ccc(Oc5ccc6c(c5)nnn6C)c(C)c4F)c3n2)C1] |
| 296 | 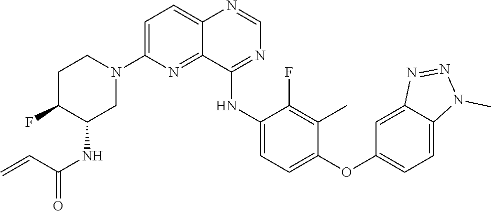 [SMILES: C=CC(=O)N[C@H]1CN(c2ccc3ncnc(Nc4ccc(Oc5ccc6c(c5)nnn6C)c(C)c4F)c3n2)CC[C@@H]1F] |
| 297 | 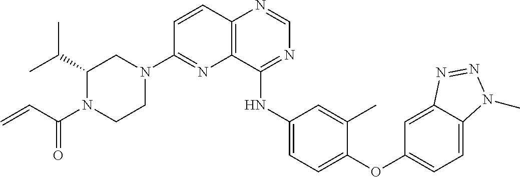 [SMILES: C=CC(=O)N1CCN(c2ccc3ncnc(Nc4ccc(Oc5ccc6c(c5)nnn6C)c(C)c4)c3n2)C[C@H]1C(C)C] |
| 298 | 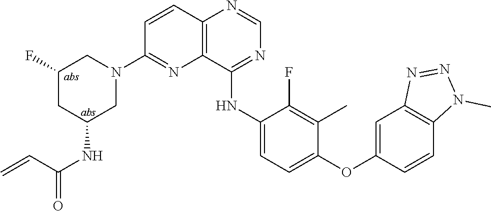 [SMILES: C=CC(=O)N[C@@H]1C[C@H](F)CN(c2ccc3ncnc(Nc4ccc(Oc5ccc6c(c5)nnn6C)c(C)c4F)c3n2)C1] |
| 299 | 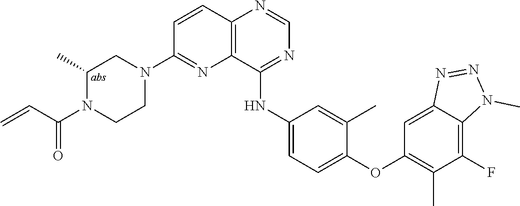 [SMILES: C=CC(=O)N1CCN(c2ccc3ncnc(Nc4ccc(Oc5cc6nnn(C)c6c(F)c5C)c(C)c4)c3n2)C[C@H]1C] |
| 300 | 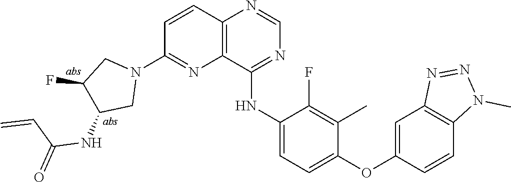 [SMILES: C=CC(=O)N[C@H]1CN(c2ccc3ncnc(Nc4ccc(Oc5ccc6c(c5)nnn6C)c(C)c4F)c3n2)C[C@@H]1F] |
| 301 | 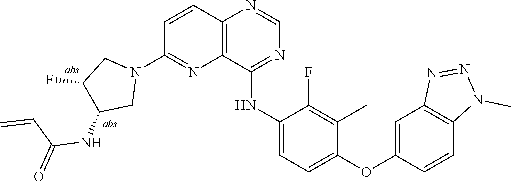 [SMILES: C=CC(=O)N[C@H]1CN(c2ccc3ncnc(Nc4ccc(Oc5ccc6c(c5)nnn6C)c(C)c4F)c3n2)C[C@H]1F] |
| 302 | 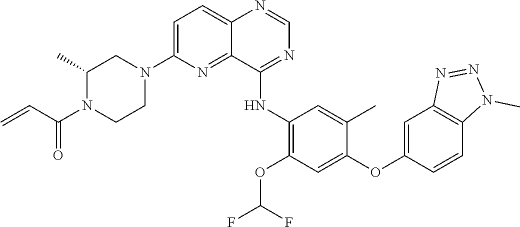 [SMILES: C=CC(=O)N1CCN(c2ccc3ncnc(Nc4cc(C)c(Oc5ccc6c(c5)nnn6C)cc4OC(F)F)c3n2)C[C@H]1C] |
| 303 | 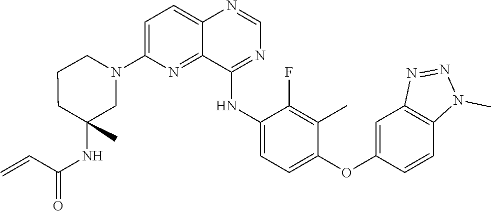 [SMILES: C=CC(=O)N[C@]1(C)CCCN(c2ccc3ncnc(Nc4ccc(Oc5ccc6c(c5)nnn6C)c(C)c4F)c3n2)C1] |
| 304 | 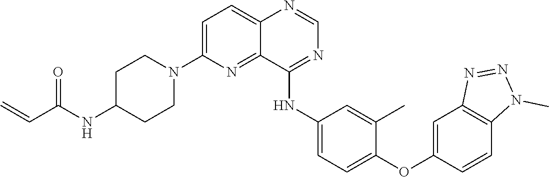 [SMILES: C=CC(=O)NC1CCN(c2ccc3ncnc(Nc4ccc(Oc5ccc6c(c5)nnn6C)c(C)c4)c3n2)CC1] |
| 305 | 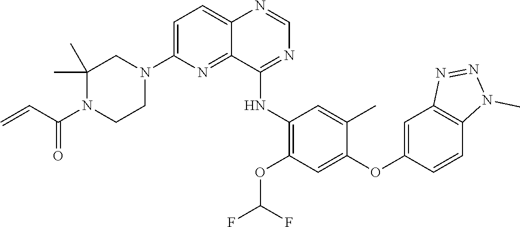 [SMILES: C=CC(=O)N1CCN(c2ccc3ncnc(Nc4cc(C)c(Oc5ccc6c(c5)nnn6C)cc4OC(F)F)c3n2)CC1(C)C] |
| 306 | 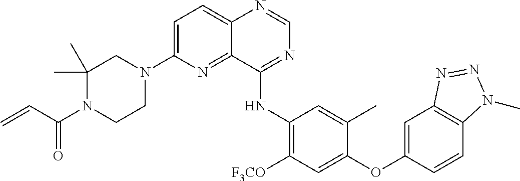 [SMILES: C=CC(=O)N1CCN(c2ccc3ncnc(Nc4cc(C)c(Oc5ccc6c(c5)nnn6C)cc4OC(F)(F)F)c3n2)CC1(C)C] |
| 307 | 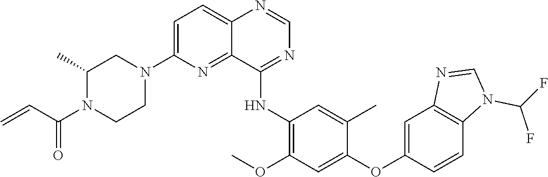 [SMILES: C=CC(=O)N1CCN(c2ccc3ncnc(Nc4cc(C)c(Oc5ccc6c(c5)ncn6C(F)F)cc4OC)c3n2)C[C@H]1C] |
| 308 | 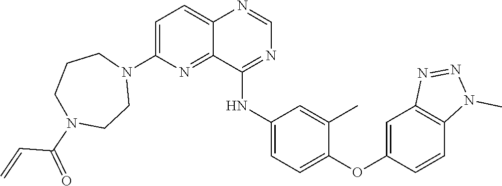 [SMILES: C=CC(=O)N1CCCN(c2ccc3ncnc(Nc4ccc(Oc5ccc6c(c5)nnn6C)c(C)c4)c3n2)CC1] |
| 309 | 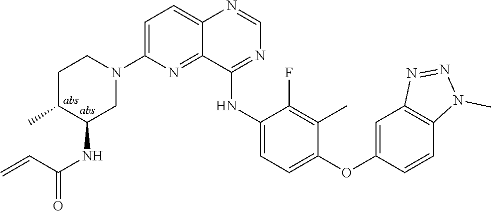 [SMILES: C=CC(=O)N[C@@H]1CN(c2ccc3ncnc(Nc4ccc(Oc5ccc6c(c5)nnn6C)c(C)c4F)c3n2)CC[C@H]1C] |
| 310 | 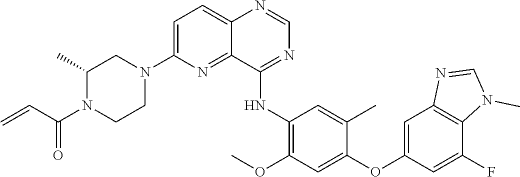 [SMILES: C=CC(=O)N1CCN(c2ccc3ncnc(Nc4cc(C)c(Oc5cc(F)c6c(c5)ncn6C)cc4OC)c3n2)C[C@H]1C] |
| 311 | 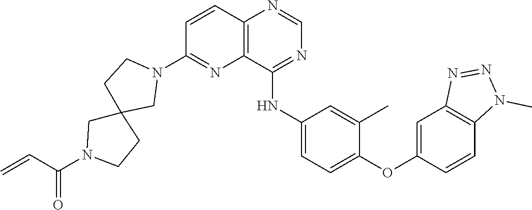 [SMILES: C=CC(=O)N1CCC2(CCN(c3ccc4ncnc(Nc5ccc(Oc6ccc7c(c6)nnn7C)c(C)c5)c4n3)C2)C1] |
| 312 | 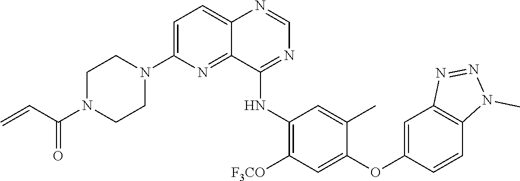 [SMILES: C=CC(=O)N1CCN(c2ccc3ncnc(Nc4cc(C)c(Oc5ccc6c(c5)nnn6C)cc4OC(F)(F)F)c3n2)CC1] |
| 313 | 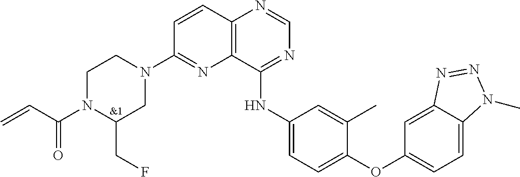 [SMILES: C=CC(=O)N1CCN(c2ccc3ncnc(Nc4ccc(Oc5ccc6c(c5)nnn6C)c(C)c4)c3n2)CC1CF] |
| 314 | 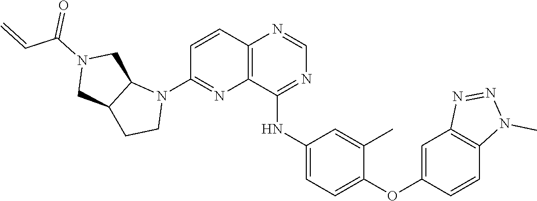 [SMILES: C=CC(=O)N1C[C@@H]2CCN(c3ccc4ncnc(Nc5ccc(Oc6ccc7c(c6)nnn7C)c(C)c5)c4n3)[C@@H]2C1] |
| 315 | 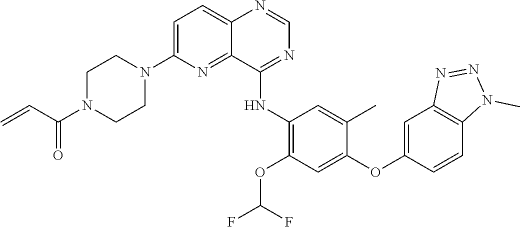 [SMILES: C=CC(=O)N1CCN(c2ccc3ncnc(Nc4cc(C)c(Oc5ccc6c(c5)nnn6C)cc4OC(F)F)c3n2)CC1] |
| 316 | 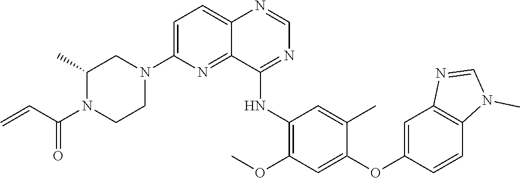 [SMILES: C=CC(=O)N1CCN(c2ccc3ncnc(Nc4cc(C)c(Oc5ccc6c(c5)ncn6C)cc4OC)c3n2)C[C@H]1C] |
| 317 | 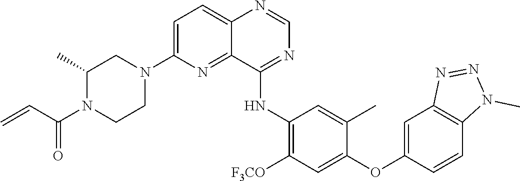 [SMILES: C=CC(=O)N1CCN(c2ccc3ncnc(Nc4cc(C)c(Oc5ccc6c(c5)nnn6C)cc4OC(F)(F)F)c3n2)C[C@H]1C] |
| 318 | 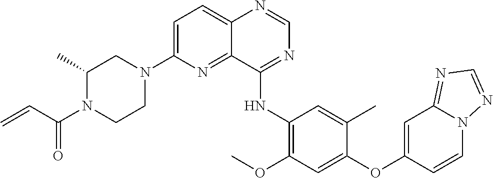 [SMILES: C=CC(=O)N1CCN(c2ccc3ncnc(Nc4cc(C)c(Oc5ccn6ncnc6c5)cc4OC)c3n2)C[C@H]1C] |
| 319 | 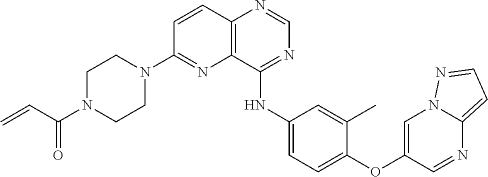 [SMILES: C=CC(=O)N1CCN(c2ccc3ncnc(Nc4ccc(Oc5cnc6ccnn6c5)c(C)c4)c3n2)CC1] |
| 320 | 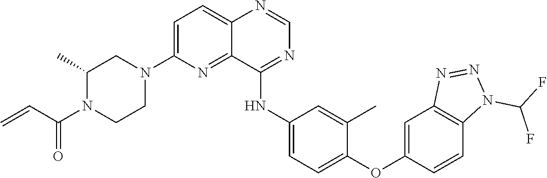 [SMILES: C=CC(=O)N1CCN(c2ccc3ncnc(Nc4ccc(Oc5ccc6c(c5)nnn6C(F)F)c(C)c4)c3n2)C[C@H]1C] |
| 321 | 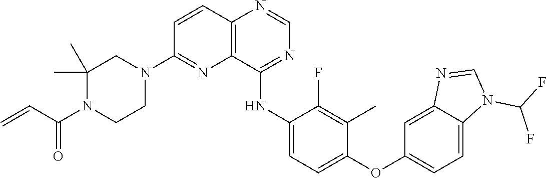 [SMILES: C=CC(=O)N1CCN(c2ccc3ncnc(Nc4ccc(Oc5ccc6c(c5)ncn6C(F)F)c(C)c4F)c3n2)CC1(C)C] |
| 322 | 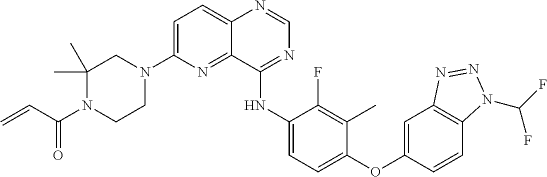 [SMILES: C=CC(=O)N1CCN(c2ccc3ncnc(Nc4ccc(Oc5ccc6c(c5)nnn6C(F)F)c(C)c4F)c3n2)CC1(C)C] |
| 323 | 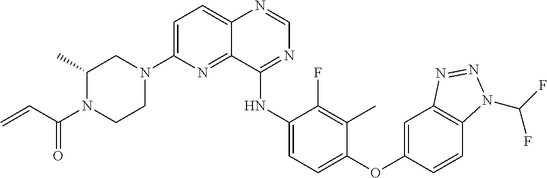 [SMILES: C=CC(=O)N1CCN(c2ccc3ncnc(Nc4ccc(Oc5ccc6c(c5)nnn6C(F)F)c(C)c4F)c3n2)C[C@H]1C] |
| 324 | 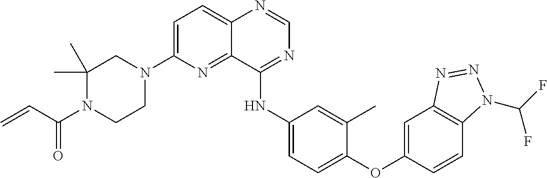 [SMILES: C=CC(=O)N1CCN(c2ccc3ncnc(Nc4ccc(Oc5ccc6c(c5)nnn6C(F)F)c(C)c4)c3n2)CC1(C)C] |
| 325 | 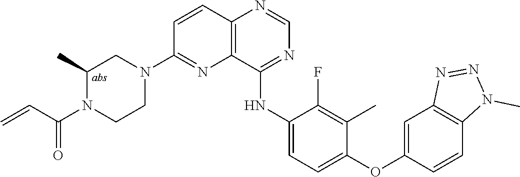 [SMILES: C=CC(=O)N1CCN(c2ccc3ncnc(Nc4ccc(Oc5ccc6c(c5)nnn6C)c(C)c4F)c3n2)C[C@@H]1C] |
| 326 | 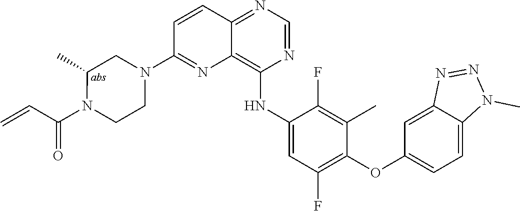 [SMILES: C=CC(=O)N1CCN(c2ccc3ncnc(Nc4cc(F)c(Oc5ccc6c(c5)nnn6C)c(C)c4F)c3n2)C[C@H]1C] |
| 327 | 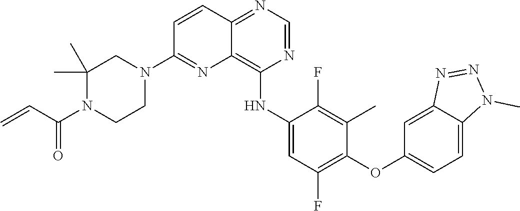 [SMILES: C=CC(=O)N1CCN(c2ccc3ncnc(Nc4cc(F)c(Oc5ccc6c(c5)nnn6C)c(C)c4F)c3n2)CC1(C)C] |
| 328 | 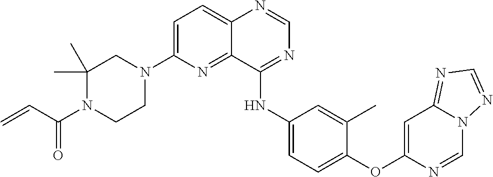 [SMILES: C=CC(=O)N1CCN(c2ccc3ncnc(Nc4ccc(Oc5cc6ncnn6cn5)c(C)c4)c3n2)CC1(C)C] |
| 329 | 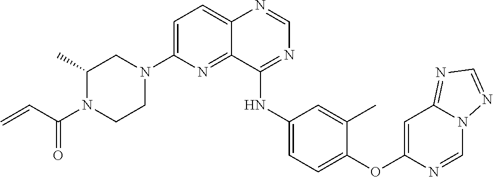 [SMILES: C=CC(=O)N1CCN(c2ccc3ncnc(Nc4ccc(Oc5cc6ncnn6cn5)c(C)c4)c3n2)C[C@H]1C] |
| 330 | 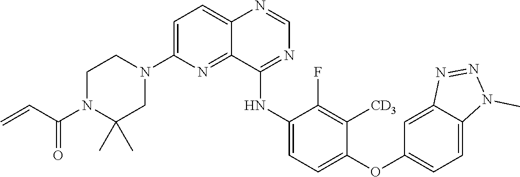 [SMILES: [2H]C([2H])([2H])c1c(Oc2ccc3c(c2)nnn3C)ccc(Nc2ncnc3ccc(N4CCN(C(=O)C=C)C(C)(C)C4)nc23)c1F] |
| 331 | 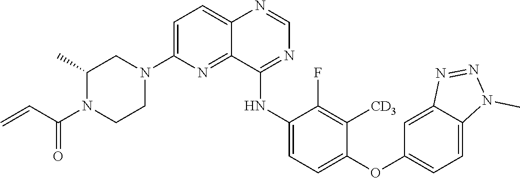 [SMILES: [2H]C([2H])([2H])c1c(Oc2ccc3c(c2)nnn3C)ccc(Nc2ncnc3ccc(N4CCN(C(=O)C=C)[C@H](C)C4)nc23)c1F] |
| 332 | 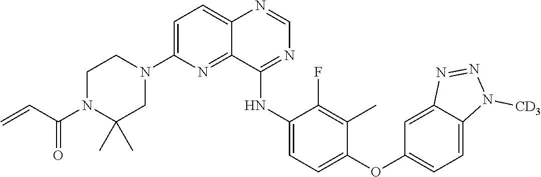 [SMILES: [2H]C([2H])([2H])n1nnc2cc(Oc3ccc(Nc4ncnc5ccc(N6CCN(C(=O)C=C)C(C)(C)C6)nc45)c(F)c3C)ccc21] |
| 333 | 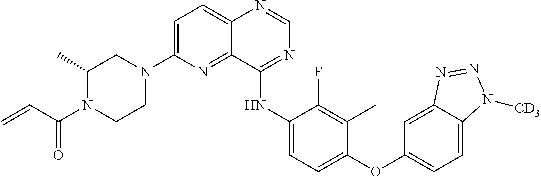 [SMILES: [2H]C([2H])([2H])n1nnc2cc(Oc3ccc(Nc4ncnc5ccc(N6CCN(C(=O)C=C)[C@H](C)C6)nc45)c(F)c3C)ccc21] |
| 334 | 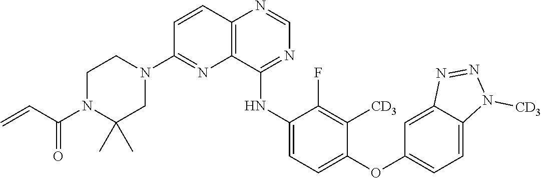 [SMILES: [2H]C([2H])([2H])c1c(Oc2ccc3c(c2)nnn3C([2H])([2H])[2H])ccc(Nc2ncnc3ccc(N4CCN(C(=O)C=C)C(C)(C)C4)nc23)c1F] |
| 335 | 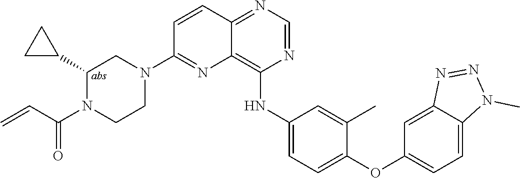 [SMILES: C=CC(=O)N1CCN(c2ccc3ncnc(Nc4ccc(Oc5ccc6c(c5)nnn6C)c(C)c4)c3n2)C[C@H]1C1CC1] |
| 336 | 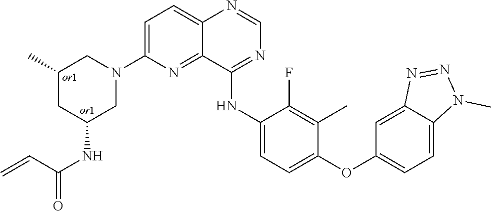 [SMILES: C=CC(=O)N[C@@H]1C[C@H](C)CN(c2ccc3ncnc(Nc4ccc(Oc5ccc6c(c5)nnn6C)c(C)c4F)c3n2)C1] |
| 337 | 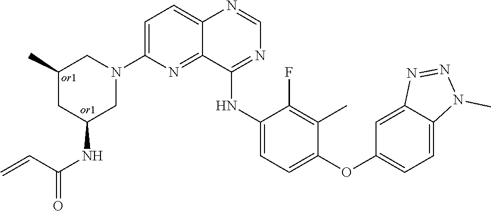 [SMILES: C=CC(=O)N[C@H]1C[C@@H](C)CN(c2ccc3ncnc(Nc4ccc(Oc5ccc6c(c5)nnn6C)c(C)c4F)c3n2)C1] |
| 338 | 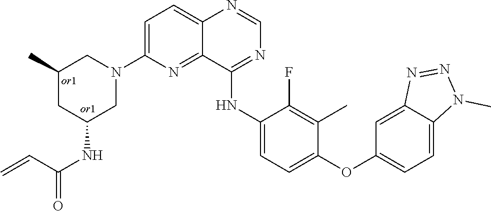 [SMILES: C=CC(=O)N[C@@H]1C[C@@H](C)CN(c2ccc3ncnc(Nc4ccc(Oc5ccc6c(c5)nnn6C)c(C)c4F)c3n2)C1] |
| 339 | 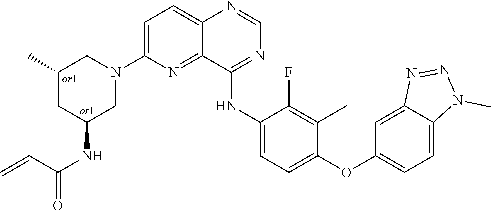 [SMILES: C=CC(=O)N[C@H]1C[C@H](C)CN(c2ccc3ncnc(Nc4ccc(Oc5ccc6c(c5)nnn6C)c(C)c4F)c3n2)C1] |
| 340 | 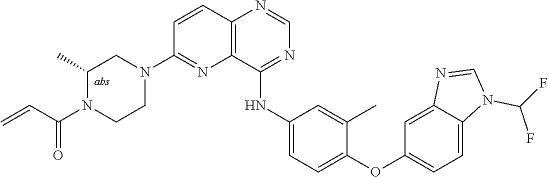 [SMILES: C=CC(=O)N1CCN(c2ccc3ncnc(Nc4ccc(Oc5ccc6c(c5)ncn6C(F)F)c(C)c4)c3n2)C[C@H]1C] |
| 341 | 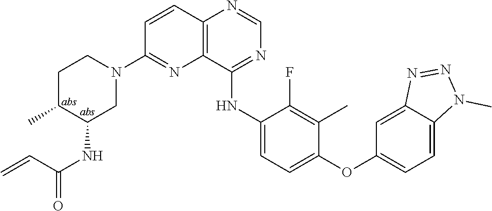 [SMILES: C=CC(=O)N[C@H]1CN(c2ccc3ncnc(Nc4ccc(Oc5ccc6c(c5)nnn6C)c(C)c4F)c3n2)CC[C@H]1C] |
| 342 | 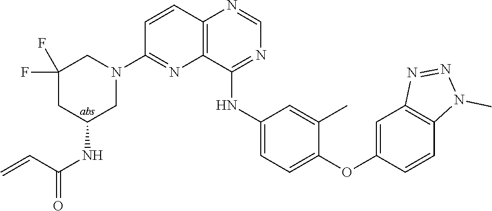 [SMILES: C=CC(=O)N[C@H]1CN(c2ccc3ncnc(Nc4ccc(Oc5ccc6c(c5)nnn6C)c(C)c4)c3n2)CC(F)(F)C1] |
| 343 | 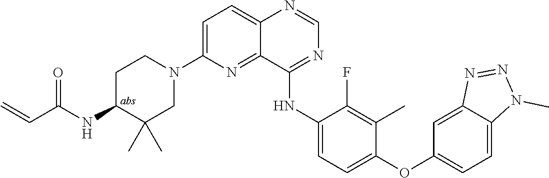 [SMILES: C=CC(=O)N[C@H]1CCN(c2ccc3ncnc(Nc4ccc(Oc5ccc6c(c5)nnn6C)c(C)c4F)c3n2)CC1(C)C] |
| 344 | 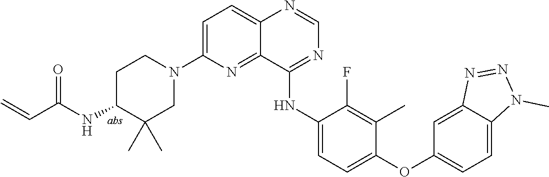 [SMILES: C=CC(=O)N[C@@H]1CCN(c2ccc3ncnc(Nc4ccc(Oc5ccc6c(c5)nnn6C)c(C)c4F)c3n2)CC1(C)C] |
| 345 | 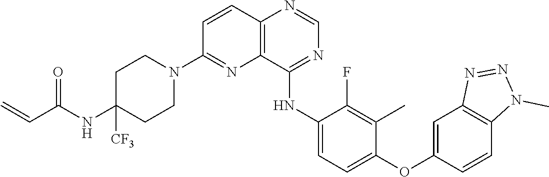 [SMILES: C=CC(=O)NC1(C(F)(F)F)CCN(c2ccc3ncnc(Nc4ccc(Oc5ccc6c(c5)nnn6C)c(C)c4F)c3n2)CC1] |
| 346 | 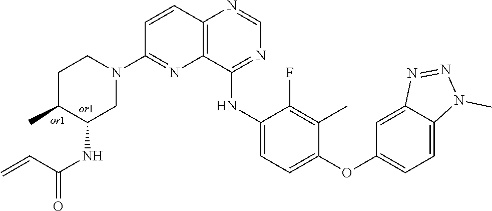 [SMILES: C=CC(=O)N[C@H]1CN(c2ccc3ncnc(Nc4ccc(Oc5ccc6c(c5)nnn6C)c(C)c4F)c3n2)CC[C@@H]1C] |
| 347 | 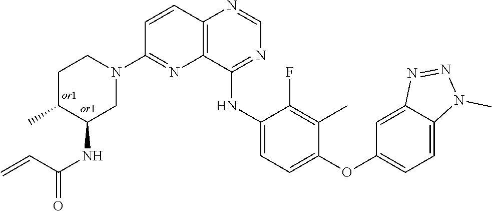 [SMILES: C=CC(=O)N[C@@H]1CN(c2ccc3ncnc(Nc4ccc(Oc5ccc6c(c5)nnn6C)c(C)c4F)c3n2)CC[C@H]1C] |
| 348 | 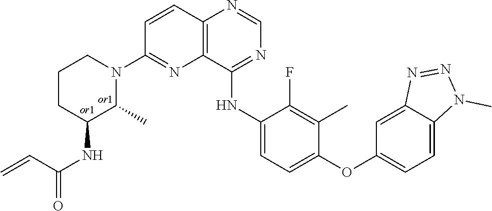 [SMILES: C=CC(=O)N[C@H]1CCCN(c2ccc3ncnc(Nc4ccc(Oc5ccc6c(c5)nnn6C)c(C)c4F)c3n2)[C@@H]1C] |
| 349 | 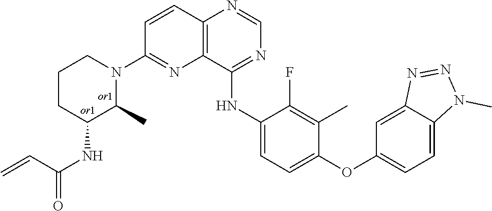 [SMILES: C=CC(=O)N[C@@H]1CCCN(c2ccc3ncnc(Nc4ccc(Oc5ccc6c(c5)nnn6C)c(C)c4F)c3n2)[C@H]1C] |
| 350 | 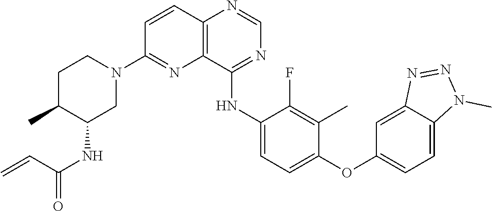 [SMILES: C=CC(=O)N[C@H]1CN(c2ccc3ncnc(Nc4ccc(Oc5ccc6c(c5)nnn6C)c(C)c4F)c3n2)CC[C@@H]1C] |
| 351 | 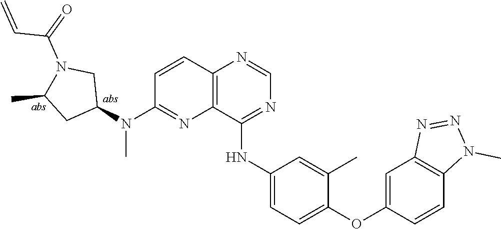 [SMILES: C=CC(=O)N1C[C@@H](N(C)c2ccc3ncnc(Nc4ccc(Oc5ccc6c(c5)nnn6C)c(C)c4)c3n2)C[C@H]1C] |
| 352 | 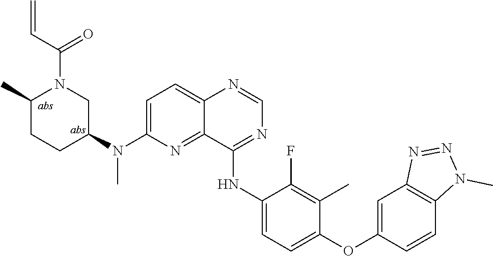 |
| 353 | 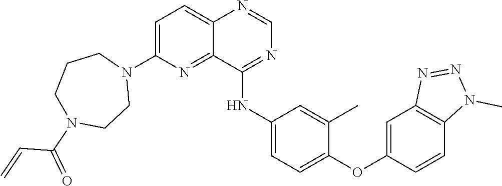 [SMILES: C=CC(=O)N1CCCN(c2ccc3ncnc(Nc4ccc(Oc5ccc6c(c5)nnn6C)c(C)c4)c3n2)CC1] |
| 354 | 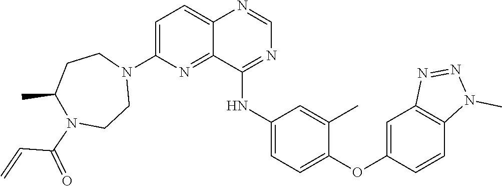 [SMILES: C=CC(=O)N1CCN(c2ccc3ncnc(Nc4ccc(Oc5ccc6c(c5)nnn6C)c(C)c4)c3n2)CC[C@@H]1C] |
| 355 | 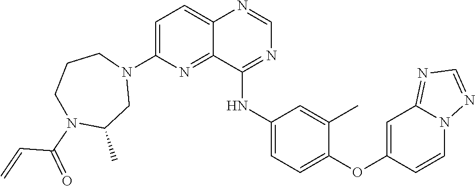 [SMILES: C=CC(=O)N1CCCN(c2ccc3ncnc(Nc4ccc(Oc5ccn6ncnc6c5)c(C)c4)c3n2)C[C@@H]1C] |
| 356 | 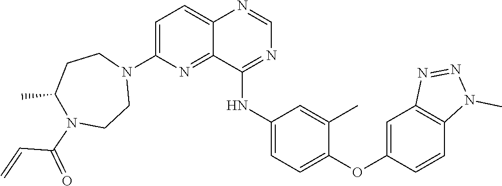 [SMILES: C=CC(=O)N1CCN(c2ccc3ncnc(Nc4ccc(Oc5ccc6c(c5)nnn6C)c(C)c4)c3n2)CC[C@H]1C] |
| 357 | 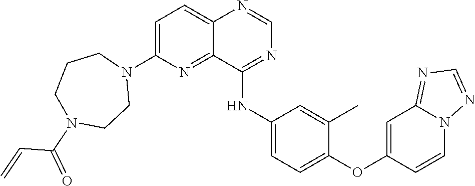 [SMILES: C=CC(=O)N1CCCN(c2ccc3ncnc(Nc4ccc(Oc5ccn6ncnc6c5)c(C)c4)c3n2)CC1] |
| 358 | 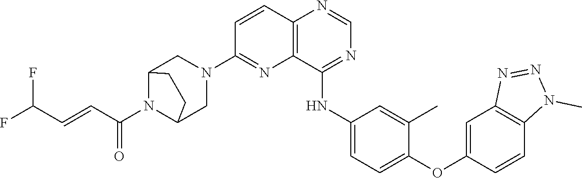 [SMILES: Cc1cc(Nc2ncnc3ccc(N4CC5CCC(C4)N5C(=O)/C=C/C(F)F)nc23)ccc1Oc1ccc2c(c1)nnn2C] |
| 359 | 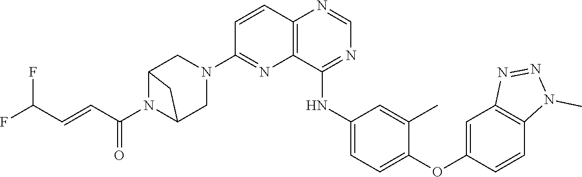 [SMILES: Cc1cc(Nc2ncnc3ccc(N4CC5CC(C4)N5C(=O)/C=C/C(F)F)nc23)ccc1Oc1ccc2c(c1)nnn2C] |
| 360 | 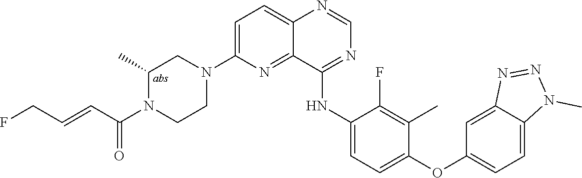 [SMILES: Cc1c(Oc2ccc3c(c2)nnn3C)ccc(Nc2ncnc3ccc(N4CCN(C(=O)/C=C/CF)[C@H](C)C4)nc23)c1F] |
| 361 | 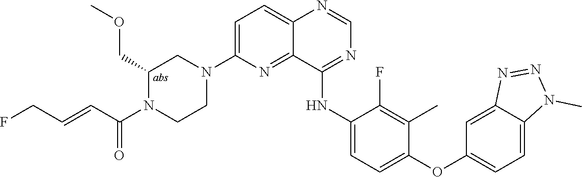 [SMILES: COC[C@@H]1CN(c2ccc3ncnc(Nc4ccc(Oc5ccc6c(c5)nnn6C)c(C)c4F)c3n2)CCN1C(=O)/C=C/CF] |
| 362 | 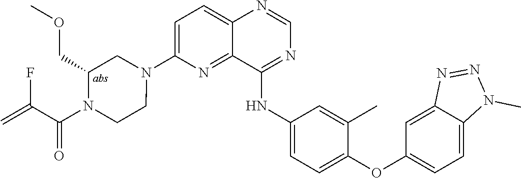 [SMILES: C=C(F)C(=O)N1CCN(c2ccc3ncnc(Nc4ccc(Oc5ccc6c(c5)nnn6C)c(C)c4)c3n2)C[C@H]1COC] |
| 363 | 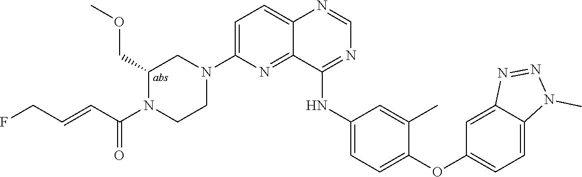 [SMILES: COC[C@@H]1CN(c2ccc3ncnc(Nc4ccc(Oc5ccc6c(c5)nnn6C)c(C)c4)c3n2)CCN1C(=O)/C=C/CF] |
| 364 | 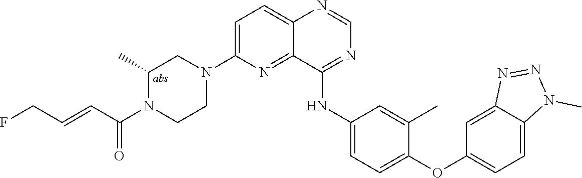 [SMILES: Cc1cc(Nc2ncnc3ccc(N4CCN(C(=O)/C=C/CF)[C@H](C)C4)nc23)ccc1Oc1ccc2c(c1)nnn2C] |
| 365 | 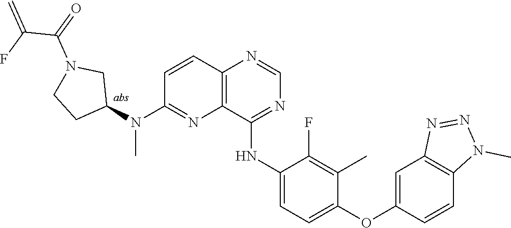 [SMILES: C=C(F)C(=O)N1CC[C@H](N(C)c2ccc3ncnc(Nc4ccc(Oc5ccc6c(c5)nnn6C)c(C)c4F)c3n2)C1] |
| 366 | 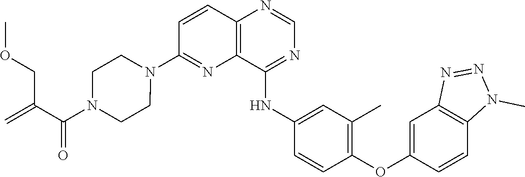 [SMILES: C=C(COC)C(=O)N1CCN(c2ccc3ncnc(Nc4ccc(Oc5ccc6c(c5)nnn6C)c(C)c4)c3n2)CC1] |
| 367 | 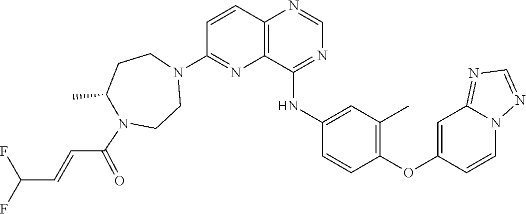 [SMILES: Cc1cc(Nc2ncnc3ccc(N4CC[C@@H](C)N(C(=O)/C=C/C(F)F)CC4)nc23)ccc1Oc1ccn2ncnc2c1] |
| 368 | 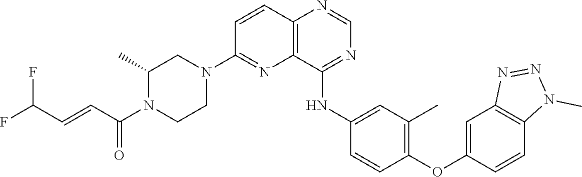 [SMILES: Cc1cc(Nc2ncnc3ccc(N4CCN(C(=O)/C=C/C(F)F)[C@H](C)C4)nc23)ccc1Oc1ccc2c(c1)nnn2C] |
| 369 | 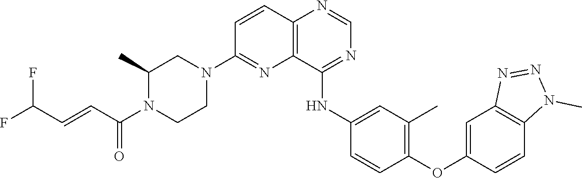 [SMILES: Cc1cc(Nc2ncnc3ccc(N4CCN(C(=O)/C=C/C(F)F)[C@@H](C)C4)nc23)ccc1Oc1ccc2c(c1)nnn2C] |
| 370 | 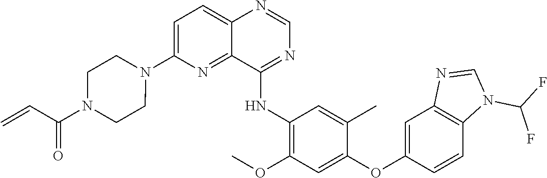 [SMILES: C=CC(=O)N1CCN(c2ccc3ncnc(Nc4cc(C)c(Oc5ccc6c(c5)ncn6C(F)F)cc4OC)c3n2)CC1] |
| 371 | 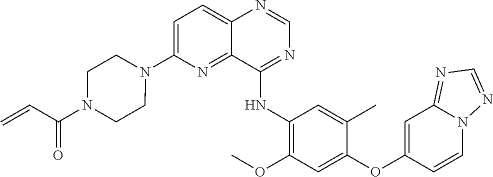 [SMILES: C=CC(=O)N1CCN(c2ccc3ncnc(Nc4cc(C)c(Oc5ccn6ncnc6c5)cc4OC)c3n2)CC1] |
| 372 | 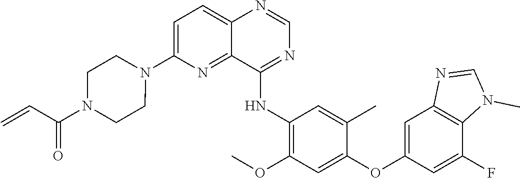 [SMILES: C=CC(=O)N1CCN(c2ccc3ncnc(Nc4cc(C)c(Oc5cc(F)c6c(c5)ncn6C)cc4OC)c3n2)CC1] |
| 373 | 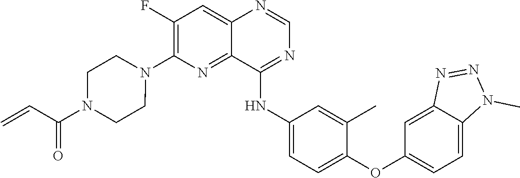 [SMILES: C=CC(=O)N1CCN(c2nc3c(Nc4ccc(Oc5ccc6c(c5)nnn6C)c(C)c4)ncnc3cc2F)CC1] |
| 374 | 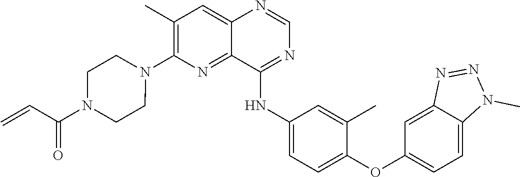 [SMILES: C=CC(=O)N1CCN(c2nc3c(Nc4ccc(Oc5ccc6c(c5)nnn6C)c(C)c4)ncnc3cc2C)CC1] |
| 375 | 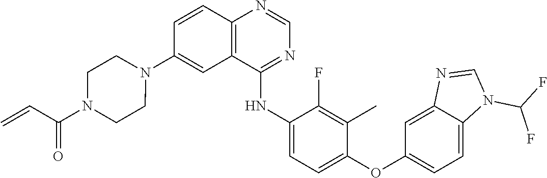 [SMILES: C=CC(=O)N1CCN(c2ccc3ncnc(Nc4ccc(Oc5ccc6c(c5)ncn6C(F)F)c(C)c4F)c3c2)CC1] |
| 376 | 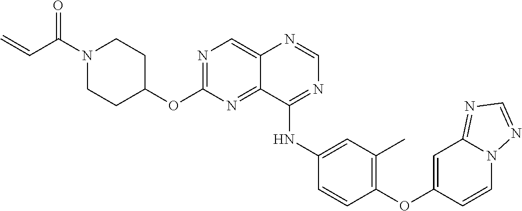 [SMILES: C=CC(=O)N1CCC(Oc2ncc3ncnc(Nc4ccc(Oc5ccn6ncnc6c5)c(C)c4)c3n2)CC1] |
| 377 | 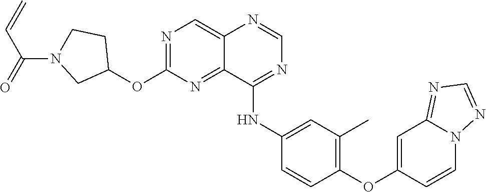 [SMILES: C=CC(=O)N1CCC(Oc2ncc3ncnc(Nc4ccc(Oc5ccn6ncnc6c5)c(C)c4)c3n2)C1] |
| 378 | 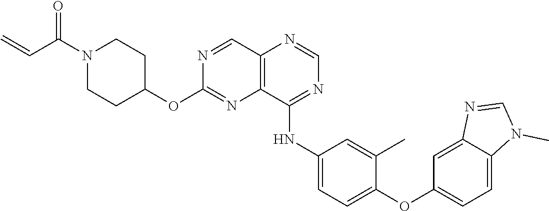 [SMILES: C=CC(=O)N1CCC(Oc2ncc3ncnc(Nc4ccc(Oc5ccc6c(c5)ncn6C)c(C)c4)c3n2)CC1] |
| 379 | 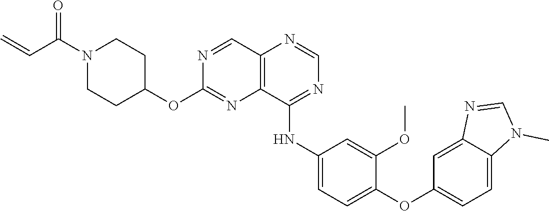 [SMILES: C=CC(=O)N1CCC(Oc2ncc3ncnc(Nc4ccc(Oc5ccc6c(c5)ncn6C)c(OC)c4)c3n2)CC1] |
| 380 | 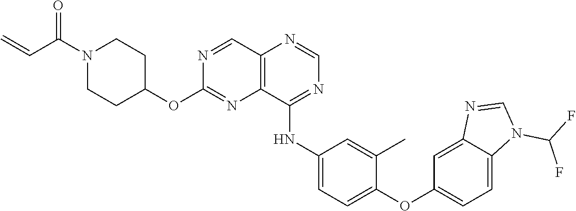 [SMILES: C=CC(=O)N1CCC(Oc2ncc3ncnc(Nc4ccc(Oc5ccc6c(c5)ncn6C(F)F)c(C)c4)c3n2)CC1] |
| 381 | 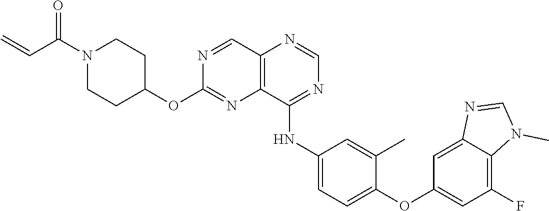 [SMILES: C=CC(=O)N1CCC(Oc2ncc3ncnc(Nc4ccc(Oc5cc(F)c6c(c5)ncn6C)c(C)c4)c3n2)CC1] |
| 382 | 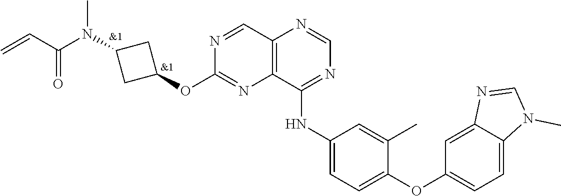 [SMILES: C=CC(=O)N(C)[C@H]1C[C@H](Oc2ncc3ncnc(Nc4ccc(Oc5ccc6c(c5)ncn6C)c(C)c4)c3n2)C1] |
| 383 | 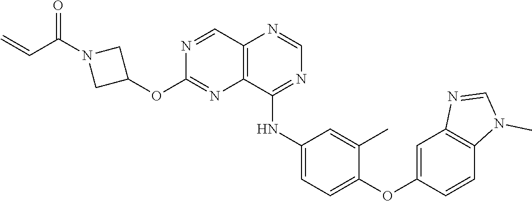 [SMILES: C=CC(=O)N1CC(Oc2ncc3ncnc(Nc4ccc(Oc5ccc6c(c5)ncn6C)c(C)c4)c3n2)C1] |
| 384 | 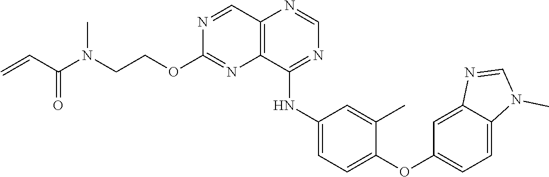 [SMILES: C=CC(=O)N(C)CCOc1ncc2ncnc(Nc3ccc(Oc4ccc5c(c4)ncn5C)c(C)c3)c2n1] |
| 385 | 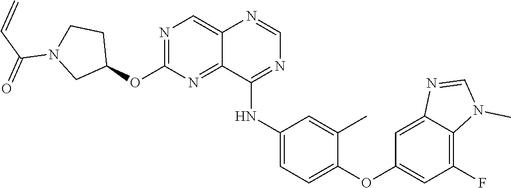 [SMILES: C=CC(=O)N1CC[C@@H](Oc2ncc3ncnc(Nc4ccc(Oc5cc(F)c6c(c5)ncn6C)c(C)c4)c3n2)C1] |
| 386 | 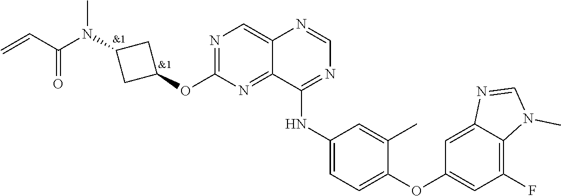 [SMILES: C=CC(=O)N(C)[C@H]1C[C@H](Oc2ncc3ncnc(Nc4ccc(Oc5cc(F)c6c(c5)ncn6C)c(C)c4)c3n2)C1] |
| 387 | 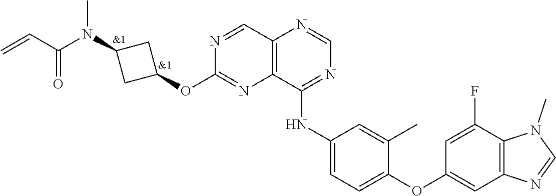 [SMILES: C=CC(=O)N(C)[C@H]1C[C@@H](Oc2ncc3ncnc(Nc4ccc(Oc5cc(F)c6c(c5)ncn6C)c(C)c4)c3n2)C1] |
| 388 | 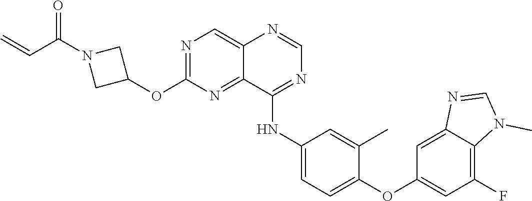 [SMILES: C=CC(=O)N1CC(Oc2ncc3ncnc(Nc4ccc(Oc5cc(F)c6c(c5)ncn6C)c(C)c4)c3n2)C1] |
| 389 | 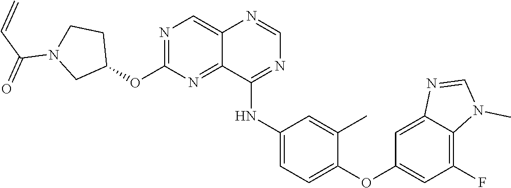 [SMILES: C=CC(=O)N1CC[C@H](Oc2ncc3ncnc(Nc4ccc(Oc5cc(F)c6c(c5)ncn6C)c(C)c4)c3n2)C1] |
| 390 | 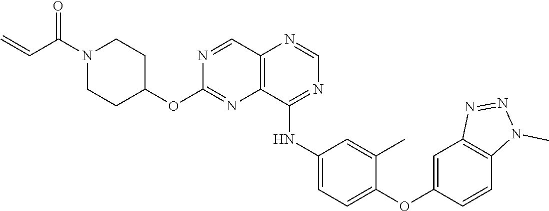 [SMILES: C=CC(=O)N1CCC(Oc2ncc3ncnc(Nc4ccc(Oc5ccc6c(c5)nnn6C)c(C)c4)c3n2)CC1] |
| 391 | 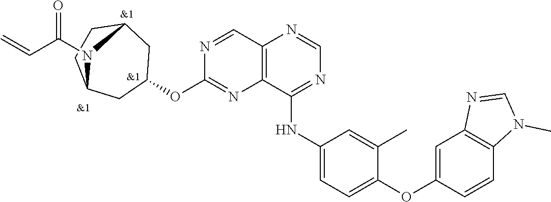 [SMILES: C=CC(=O)N1[C@@H]2CC[C@H]1C[C@@H](Oc1ncc3ncnc(Nc4ccc(Oc5ccc6c(c5)ncn6C)c(C)c4)c3n1)C2] |
| 392 | 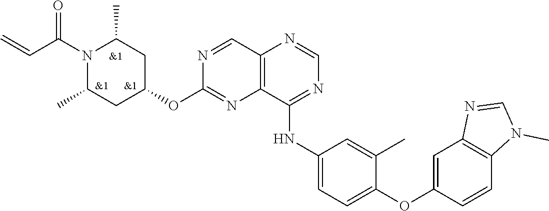 [SMILES: C=CC(=O)N1[C@H](C)C[C@H](Oc2ncc3ncnc(Nc4ccc(Oc5ccc6c(c5)ncn6C)c(C)c4)c3n2)C[C@@H]1C] |
| 393 | 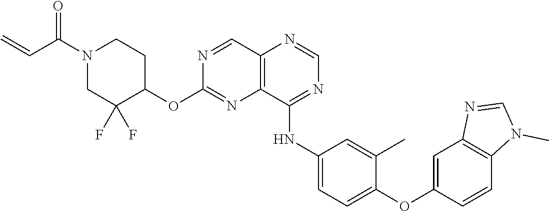 [SMILES: C=CC(=O)N1CCC(Oc2ncc3ncnc(Nc4ccc(Oc5ccc6c(c5)ncn6C)c(C)c4)c3n2)C(F)(F)C1] |
| 394 | 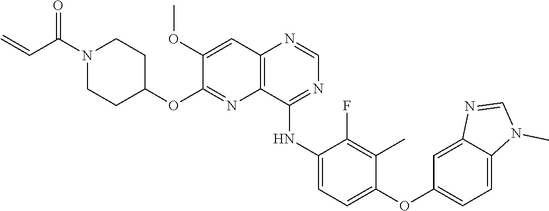 [SMILES: C=CC(=O)N1CCC(Oc2nc3c(Nc4ccc(Oc5ccc6c(c5)ncn6C)c(C)c4F)ncnc3cc2OC)CC1] |
| 395 | 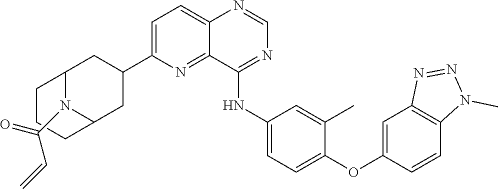 [SMILES: C=CC(=O)N1C2CCCC1CC(c1ccc3ncnc(Nc4ccc(Oc5ccc6c(c5)nnn6C)c(C)c4)c3n1)C2] |
| 396 | 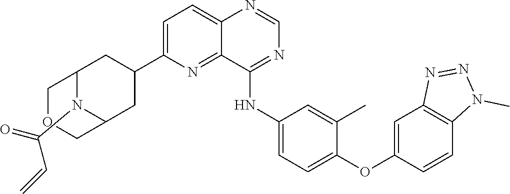 [SMILES: C=CC(=O)N1C2COCC1CC(c1ccc3ncnc(Nc4ccc(Oc5ccc6c(c5)nnn6C)c(C)c4)c3n1)C2] |
| 397 | 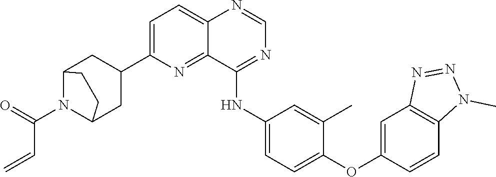 [SMILES: C=CC(=O)N1C2CCC1CC(c1ccc3ncnc(Nc4ccc(Oc5ccc6c(c5)nnn6C)c(C)c4)c3n1)C2] |
| 398 | 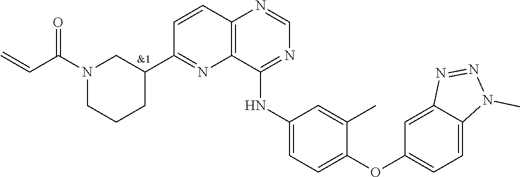 [SMILES: C=CC(=O)N1CCCC(c2ccc3ncnc(Nc4ccc(Oc5ccc6c(c5)nnn6C)c(C)c4)c3n2)C1] |
| 399 | 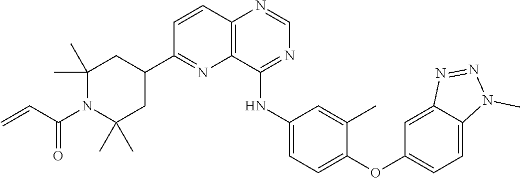 [SMILES: C=CC(=O)N1C(C)(C)CC(c2ccc3ncnc(Nc4ccc(Oc5ccc6c(c5)nnn6C)c(C)c4)c3n2)CC1(C)C] |
| 400 | 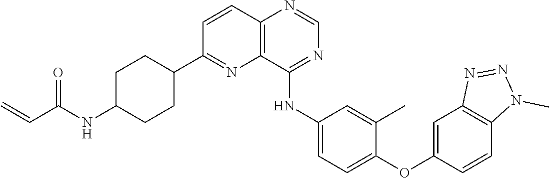 [SMILES: C=CC(=O)NC1CCC(c2ccc3ncnc(Nc4ccc(Oc5ccc6c(c5)nnn6C)c(C)c4)c3n2)CC1] |
| 401 | 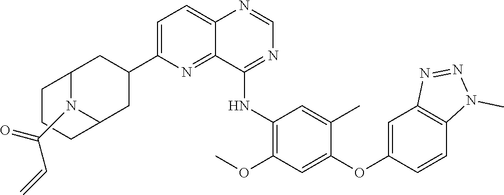 [SMILES: C=CC(=O)N1C2CCCC1CC(c1ccc3ncnc(Nc4cc(C)c(Oc5ccc6c(c5)nnn6C)cc4OC)c3n1)C2] |
| 402 | 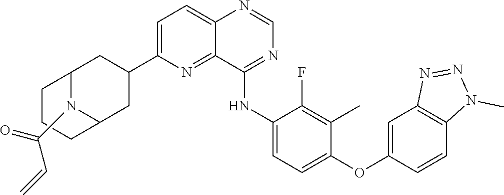 [SMILES: C=CC(=O)N1C2CCCC1CC(c1ccc3ncnc(Nc4ccc(Oc5ccc6c(c5)nnn6C)c(C)c4F)c3n1)C2] |
| 403 | 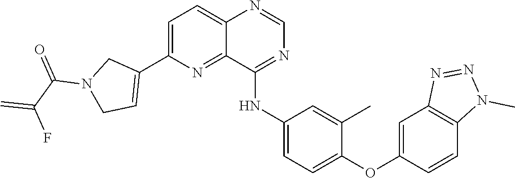 [SMILES: C=C(F)C(=O)N1CC=C(c2ccc3ncnc(Nc4ccc(Oc5ccc6c(c5)nnn6C)c(C)c4)c3n2)C1] |
| 404 | 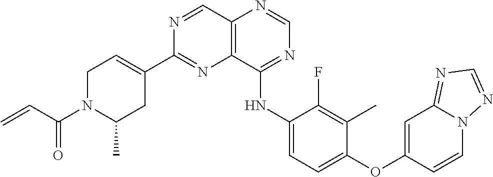 [SMILES: C=CC(=O)N1CC=C(c2ncc3ncnc(Nc4ccc(Oc5ccn6ncnc6c5)c(C)c4F)c3n2)C[C@@H]1C] |
| 405 | 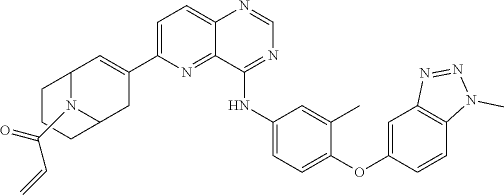 [SMILES: C=CC(=O)N1C2C=C(c3ccc4ncnc(Nc5ccc(Oc6ccc7c(c6)nnn7C)c(C)c5)c4n3)CC1CCC2] |
| 406 | 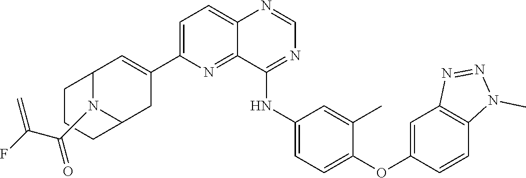 [SMILES: C=C(F)C(=O)N1C2C=C(c3ccc4ncnc(Nc5ccc(Oc6ccc7c(c6)nnn7C)c(C)c5)c4n3)CC1CCC2] |
| 407 | 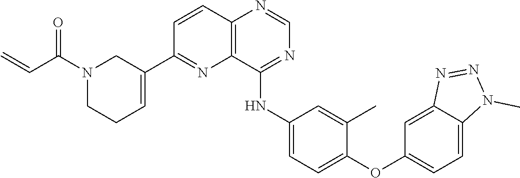 [SMILES: C=CC(=O)N1CCC=C(c2ccc3ncnc(Nc4ccc(Oc5ccc6c(c5)nnn6C)c(C)c4)c3n2)C1] |
| 408 | 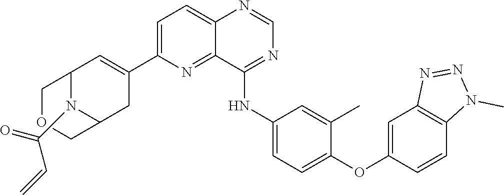 [SMILES: C=CC(=O)N1C2C=C(c3ccc4ncnc(Nc5ccc(Oc6ccc7c(c6)nnn7C)c(C)c5)c4n3)CC1COC2] |
| 409 | 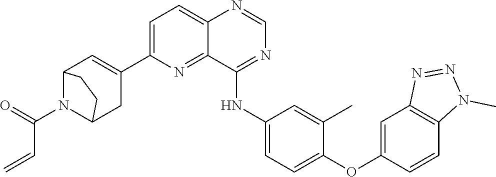 [SMILES: C=CC(=O)N1C2C=C(c3ccc4ncnc(Nc5ccc(Oc6ccc7c(c6)nnn7C)c(C)c5)c4n3)CC1CC2] |
| 410 | 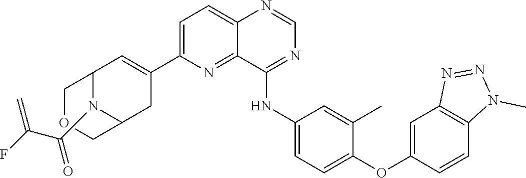 [SMILES: C=C(F)C(=O)N1C2C=C(c3ccc4ncnc(Nc5ccc(Oc6ccc7c(c6)nnn7C)c(C)c5)c4n3)CC1COC2] |
| 411 | 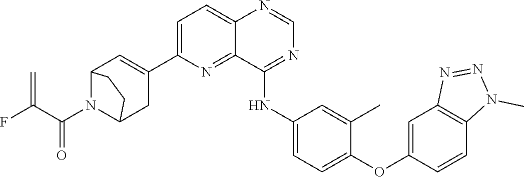 [SMILES: C=C(F)C(=O)N1C2C=C(c3ccc4ncnc(Nc5ccc(Oc6ccc7c(c6)nnn7C)c(C)c5)c4n3)CC1CC2] |
| 412 | 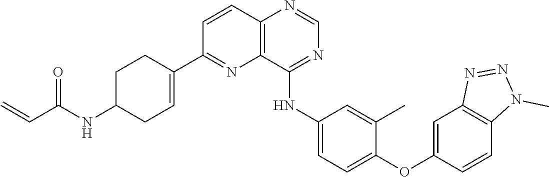 [SMILES: C=CC(=O)NC1CC=C(c2ccc3ncnc(Nc4ccc(Oc5ccc6c(c5)nnn6C)c(C)c4)c3n2)CC1] |
| 413 | 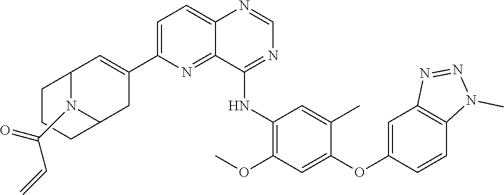 [SMILES: C=CC(=O)N1C2C=C(c3ccc4ncnc(Nc5cc(C)c(Oc6ccc7c(c6)nnn7C)cc5OC)c4n3)CC1CCC2] |
| 414 | 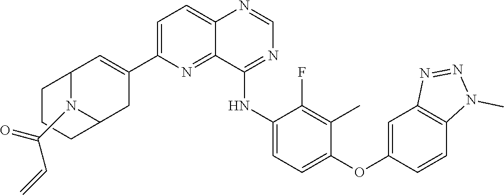 [SMILES: C=CC(=O)N1C2C=C(c3ccc4ncnc(Nc5ccc(Oc6ccc7c(c6)nnn7C)c(C)c5F)c4n3)CC1CCC2] |

As can be appreciated, the compounds described herein enable the development of new therapies for disease without the need for exotic chemistry or specialized reagents or manufacturing techniques.

### III Pharmaceutical Compositions

Other embodiments are directed to pharmaceutical compositions. In an embodiment, the pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such additional therapeutic agents are described herein below.

Suitable routes of administration include, but are not limited to, oral, intravenous, rectal, aerosol, parenteral, ophthalmic, pulmonary, transmucosal, transdermal, vaginal, optical, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with an organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Formula (I), (IA)-(ID), (IA-1), (IA-2), (IB-1), or (IB-2) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from about 0.001 to 0.1 mg, 0.01 to 0.1 mg, 0.5 to 5 mg, 0.5 to 10 mg, 0.01-10 mg, 0.1 to 10 mg, 10 to 5000 mg, 100 to 5000 mg, 1000 mg to 4000 mg per day, or 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. In an embodiment, the single dose is administered orally. In another embodiment, the single dose is administered by injection. However, other routes are used as appropriate. In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., an additional anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Provided herein are pharmaceutical compositions comprising one or more compounds of Formula (I), (IA)-(ID), (IA-1), (IA-2), (IB-1), or (IB-2), and a pharmaceutically acceptable carrier. Also provided herein are pharmaceutical compositions comprising one or more compounds selected from compounds of Formula (I)—(IL) and pharmaceutically acceptable diluent(s), excipient(s), and carrier(s). In certain embodiments, the compounds described are administered as pharmaceutical compositions in which one or more compounds selected from compounds of Formula (1), (IA)-(ID), (IA-1), (IA-2), (IB-1), or (113-2) are mixed with other active ingredients, as in combination therapy. Encompassed herein are all combinations of actives set forth in the combination therapies section below and throughout this disclosure. In specific embodiments, the pharmaceutical compositions include one or more compounds of Formula (1), (IA)-(ID), (IA-1), (IA-2), (IB-1), or (IB-2).

A pharmaceutical composition, as used herein, refers to a mixture of one or more compounds selected from compounds of Formula (I). (IA)-(ID), (IA-1), (IA-2), (IB-1), or (113-2) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Formula (I), (IA)-(ID), (IA-1), (IA-2), (IB-1), or (IB-2) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Formula (I), (IA)-(ID), (IA-I), (IA-2), (IB—I), or (IB-2) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Formula (I), (IA)-(ID), (IA-1), (IA-2), (IB-1), or (IB-2) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a pharmaceutically acceptable salt thereof such as sodium alginate.

In one embodiment, the oral dosage forms, such as a pill, capsule or tablet, comprises one or more suitable layers or coatings. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of one or more compounds selected from compounds of Formula (I), (IA)-(ID), (IA-1), (IA-2), (IB-1), or (IB-2) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Formula (I), (IA)-(ID), (IA-1), (IA-2), (IB-1), or (IB-2) as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, pharmaceutically acceptable salts for regulating the osmotic pressure, buffers, and/or other therapeutically valuable substances.

Methods for the preparation of compositions comprising the compounds described herein include formulating the compound(s) with one or more inert, pharmaceutically acceptable excipients or carriers to form a solid, semi-solid or liquid composition. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, ointments, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Formula (I), (IA)-(ID), (IA-1), (IA-2), (IB-1), or (IB-2) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Formula (I), (IA)-(ID), (IA-1) (IA-2), (IB-1), or (IB-2). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more pharmaceutically acceptable salts in an amount required to bring osmolality of the composition into an acceptable range. Such pharmaceutically acceptable salts include those having sodium, potassium or ammonium cations and chloride, citrate, ascorbate, borate, phosphate, bicarbonate, sulfate, thiosulfate or bisulfite anions; suitable pharmaceutically acceptable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained release system, such as semipermeable matrices of solid hydrophobic polymers containing the therapeutic agent. Various sustained release materials are useful herein. In some embodiments, sustained release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Formula (I), (IA)-(ID), (IA-1), (IA-2), (IB-1), or (IB-2) provided in the pharmaceutical compositions is greater than 90%, 80%, 75%, 70%, 65%, 60%, 55%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25%, 7%, 6.75%, 6.50%, 6.25% 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v. In another embodiment, the amount of a compound selected from compounds of Formula (I), (IA)-(ID), (IA-1), (IA-2), (IB-1), or (IB-2) in the pharmaceutical compositions is an amount between about any two of the values recited in the preceding sentence, for example, between about 2-70 w/w %, 3.5-80 w/w %, 1-30 w/w %, etc.

In some embodiments, the concentration of one or more compounds selected from compounds of Formula (I), (IA)-(ID), (IA-1), (IA-2), (IB-1), or (IB-2) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Formula (I), (IA)-(ID), (IA-1), (IA-2), (IB-1), or (IB-2) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Formula (I), (IA)-(ID), (IA-1), (IA-2), (IB-1), or (IB-2) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Alternatively, the pack or dispenser device is accompanied by instructions for administration, or the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

As mentioned above, the compounds and compositions of the disclosure will find utility in a broad range of diseases and conditions mediated by protein kinases, including diseases and conditions mediated by kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colon-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

In some embodiments, a pharmaceutical composition has a compound described above and a pharmaceutically acceptable carrier including, for example, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, a pharmaceutical composition comprising the compound of Formula (I), (IA)-(ID), (IA-1), (IA-2), (IB-1), or (IB-2) and an additional therapeutic agent is disclosed.

In one embodiment, the compound of Formula (I), (IA)-(ID), (IA-1), (IA-2), (IB-1), or (IB-2) or the pharmaceutical composition comprising the compound of Formula (I), (IA)-(ID), (IA-1), (IA-2), (IB-1), or (IB-2) and an additional therapeutic agent for use in treating a disease associated with mutations in a human epidermal growth factor receptor 2 (ErbB2) is disclosed.

In one embodiment, a method of treating a disease associated with mutations in ErbB2, comprises administering the compound of Formula (I), (IA)-(ID), (IA-1), (IA-2), (IB-1), or (IB-2) or the pharmaceutical composition comprising the compound of Formula (I), (IA)-(ID), (IA-1), (IA-2), (IB-1), or (IB-2) to a subject in need thereof. In one embodiment, the subject is an animal. In some embodiments, the subject is a human. In some embodiments, the disease associated with mutations in ErbB2 is a cancer. For example, the cancer associated with mutations in ErbB2 includes lung, glioma, esophageal, liver, stomach, uterine, cervical, biliary tract, skin, head and neck, salivary gland, breast, pancreatic, colorectal, renal, bladder, or prostate cancer. In some embodiments, the cancer associated with mutations in ErbB2 is lung cancer. In some embodiments, the cancer associated with mutations in ErbB2 is glioma cancer. In some embodiments, the cancer associated with mutations in ErbB2 is esophageal cancer. In some embodiments, the cancer associated with mutations in ErbB2 is liver cancer. In some embodiments, the cancer associated with mutations in ErbB2 is stomach cancer. In some embodiments, the cancer associated with mutations in ErbB2 is uterine cancer. In some embodiments, the cancer associated with mutations in ErbB2 is cervical cancer. In some embodiments, the cancer associated with mutations in ErbB2 is biliary tract cancer. In some embodiments, the cancer associated with mutations in ErbB2 is skin cancer. In some embodiments, the cancer associated with mutations in ErbB2 is head and neck cancer. In some embodiments, the cancer associated with mutations in ErbB2 is salivary gland cancer. In some embodiments, the cancer associated with mutations in ErbB2 is breast cancer. In some embodiments, the cancer associated with mutations in ErbB2 is pancreatic cancer. In some embodiments, the cancer associated with mutations in ErbB2 is colorectal cancer. In some embodiments, the cancer associated with mutations in ErbB2 is renal cancer. In some embodiments, the cancer associated with mutations in ErbB2 is bladder cancer. In some embodiments, the cancer associated with mutations in ErbB2 is prostate cancer. In some certain embodiments, the cancer is non-small cell lung cancer.

### IV Methods of Preparation

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

### EXAMPLES

The following section describes abbreviations used in the examples and includes examples for making intermediate compounds I1-I178 (Examples 1-88) and examples for making compounds 1-414 (Examples 89-104).

### Abbreviations

Sat.: Saturated
Aq.: Aqueous
TEA: Triethylamine
DIPEA: N,N-Diisopropylethylamine
MeCN: Acetonitrile
TFA: 2,2,2-Trifluoroacetic acid
FA: Formic acid
DCM: Dichloromethane
Pd/C: Palladium on activated carbon
PET: Petroleum ether
DMF: N,N-Dimethylformamide
IPA: Isopropyl alcohol
EtOH: Ethanol
MeOH: Methanol
n-ButOH: Butan-1-ol
Quant.: Quantitative
HATU: 2-(3H-[1,2,3]triazolo[4,5-b]pyridin-3-yl)-1,1,3,3-tetramethylisouronium
hexafluorophosphate(V)
K₂CO₃: Potassium Carbonate
Cs₂CO₃: Cesium Carbonate
Na₂SO₄: Sodium Sulfate
MgSO₄: Magnesium Sulfate
DMF-DMA: N,N-Dimethylformamide dimethyl acetal
Pd₂(dba)₃: Tris(dibenzylideneacetone)dipalladium(0)
XantPhos: 4,5-Bis(diphenylphosphino)-9,9-dimethylxanthene
MsOH: Methanesulfonic acid
LCMS: Liquid chromatography mass spectrometry
EtOAc: Ethyl acetate
THF: Tetrahydrofuran
MeI: Methyl iodide
KOAc: Potassium acetate
DMSO: Dimethyl sulfoxide
HPLC: High-performance liquid chromatography
CuCN: Copper (I) cyanide

### Syntheses of Intermediate Compounds

### Example 1 (I1)

### 1-methyl-1H-benzo[d][1,2,3]triazol-5-ol

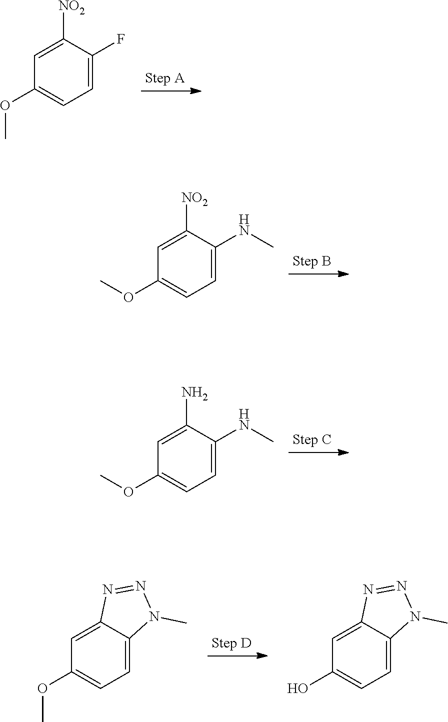

Step A. 4-methoxy-N-methyl-2-nitroaniline. Two batches in parallel each contained a solution of 1-fluoro-4-methoxy-2-nitrobenzene (45.0 g, 0.26 mol) and methylamine hydrochloride (81.7 g, 1.21 mol) in MeCN (800 mL). DIPEA (229 mL, 1.31 mol) was added and the reaction was stirred at 80° C. for 12 h. The mixture was concentrated under vacuum, and the resulting residue was diluted with water (1 L) and extracted with EtOAc (1 L×3). The organic layers were combined, washed with water (3×) and brine, dried over anhydrous Na₂SO₄, filtered, and concentrated in vacuo. The two batches were combined to provide 4-methoxy-N-methyl-2-nitro-aniline (93.6 g, 98% yield) as a red solid. LCMS (MM-ES+APCI, Pos): m/z 183.3 (M+H).

Step B. 4-methoxy-N¹-methylbenzene-1,2-diamine. To a solution of 4-methoxy-N-methyl-2-nitro-aniline (30.0 g, 165 mmol) in MeOH (400 mL) was added Pd/C (8.76 g, 8.23 mmol, 10% wt.), the reaction was stirred at 25° C. for 1 h under H₂ (3.4 atm). The mixture was filtered through diatomite and the cake was rinsed with MeOH (1 L). The filtrate was concentrated under vacuum and the residue was purified by silica gel column chromatography (10% to 15% EtOAc/PET) to give 4-methoxy-N¹-methyl-benzene-1,2-diamine (18.0 g, 72% yield) as a brown solid. LCMS (MM-ES+APCI, Pos): m/z 153.5 (M+H).

Step C. 5-methoxy-1-methyl-1H-benzo[d][1,2,3]triazole. To a solution of 4-methoxy-N¹-methyl-benzene-1,2-diamine (17.0 g, 112 mmol) in EtOH (200 mL) was added tert-butyl nitrite (15.9 mL, 134 mmol) and MsOH (15.9 mL, 223 mmol) dropwise at 0° C. The mixture was stirred at 25° C. for 1 h and concentrated in vacuo. The resulting residue was quenched by slow addition of sat. aq. NaHCO₃ (1 L), and the product was extracted with 3% MeOH/DCM (1 L×5). The combined organic layers were washed with brine (2×1 L), dried over anhydrous Na₂SO₄, filtered, and concentrated under reduced pressure. The crude material was purified by silica gel column chromatography (10% to 15% EtOAc/PET) to give 5-methoxy-1-methyl-benzotriazole (14.9 g, 82% yield) as a brown solid. LCMS (MM-ES+APCI, Pos): m/z 164.5 (M+H).

Step D. 1-methyl-1H-benzo[d][1,2,3]triazol-5-ol. To a solution of 5-methoxy-1-methyl-benzotriazole (14.9 g, 91.3 mmol) in DCM (200 mL) was added BBr₃ (13.2 mL, 137 mmol) at 0° C. The mixture was warmed to 25° C., stirred for 2 h, and quenched with water (200 mL). The mixture was neutralized (pH 7) using NaHCO₃ and extracted with DCM (200 mL×3). The combined organic layers were dried over anhydrous Na₂SO₄, filtered, and concentrated in vacuo to provide 1-methylbenzotriazol-5-ol (11.9 g, 87% yield) as a brown solid. LCMS (MM-ES+APCI, Pos): m/z 150.1 (M+H).

### Example 2 (I65)

### 1-ethyl-1H-benzo[d][1,2,3]triazol-5-ol

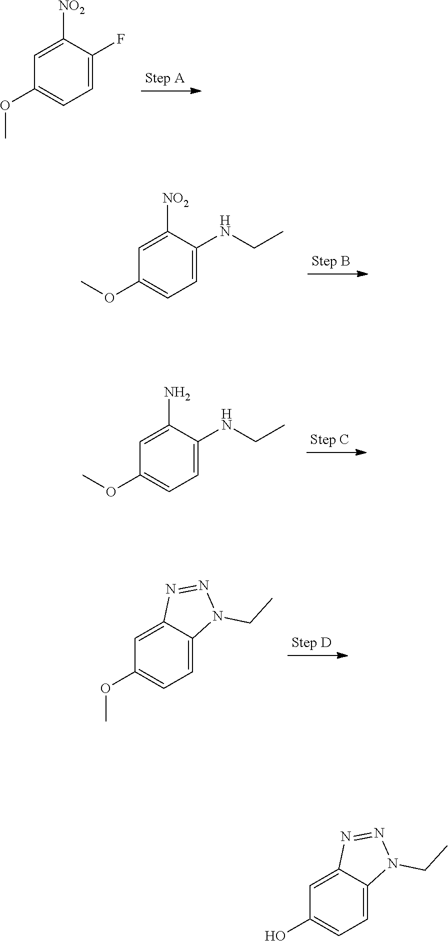

Steps A to D. 1-ethyl-1H-benzo[d][1,2,3]triazol-5-ol. Prepared according to Example 1, substituting ethanamine hydrochloride for methylamine hydrochloride to afford 1-ethyl-1H-benzo[d][1,2,3]triazol-5-ol (1.60 g, 57% yield) as a brown solid. LCMS (MM-ES+APCI, Pos): m/z 164.3 (M+H).

### Example 3 (I2)

### 7-fluoro-1-methyl-H-benzo[d][1,2,3]triazol-5-ol

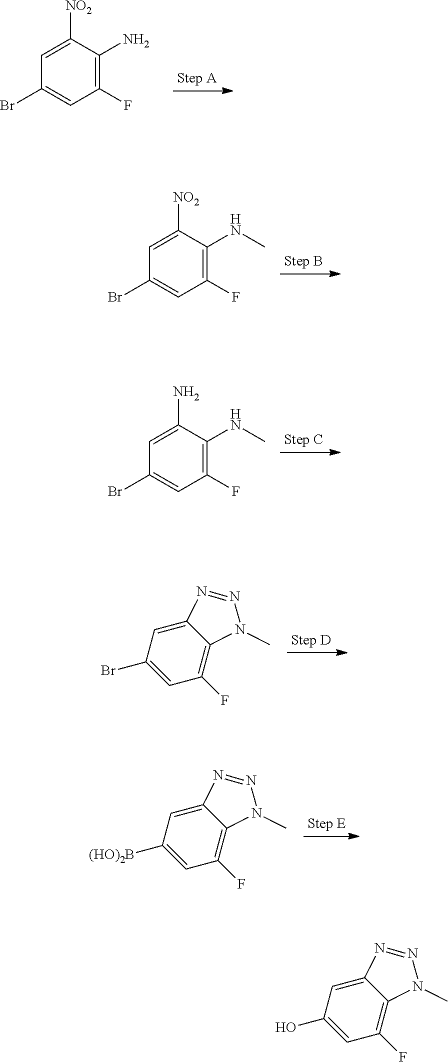

Step A. 4-bromo-2-fluoro-N-methyl-6-nitroaniline. To a solution of 4-bromo-2-fluoro-6-nitro-aniline (25.0 g, 106 mmol) in THF (250 mL) was added NaH (5.11 g, 127 mmol, 60% wt.) at 0° C. under N₂. The mixture was warmed to 25° C. for 30 min and Mel (9.93 mL, 160 mmol) was added. The reaction was stirred at 25° C. for 30 min and at 60° C. for 5 h. The reaction was quenched with sat. aq. NH₄Cl (50 mL) and extracted with EtOAc (100 mL×3). The combined organic layers were washed with brine (30 mL×3), dried over Na₂SO₄, filtered and concentrated under reduced pressure to give 4-bromo-2-fluoro-N-methyl-6-nitro-aniline (38.4 g, quant. yield) as a brown solid. LCMS (MM-ES+APCI, Pos): m/z 249.0 (M+H).

Step B. 4-bromo-6-fluoro-N¹-methylbenzene-1,2-diamine. To a solution of 4-bromo-2-fluoro-N-methyl-6-nitro-aniline (38.4 g, 77.1 mmol) in EtOH (200 mL) and H₂O (100 mL) was added NH₄Cl (41.2 g, 771 mmol) and iron powder (21.5 g, 385 mmol). The mixture was stirred at 80° C. for 2 h, cooled to 25° C., and filtered. Water (50 mL) was added to the filtrate and the product was extracted with EtOAc (500 mL×3). The combined organic layers were washed with brine (500 mL×3), dried over Na₂SO₄, filtered, and concentrated under reduced pressure. The crude residue was purified by silica gel column chromatography (15% EtOAc/PET) to afford 4-bromo-6-fluoro-N¹-methyl-benzene-1,2-diamine (14.0 g, 68% yield) as a brown oil. LCMS (MM-ES+APCI, Pos): m/z 219.0 (M+H).

Step C. 5-bromo-7-fluoro-1-methyl-1H-benzo[d][1,2,3]triazole. To a solution of 4-bromo-6-fluoro-N¹-methyl-benzene-1,2-diamine (3.00 g, 13.7 mmol) in HCl (10 mL, 12M) and water (5 mL) was added sodium nitrite (1.13 g, 16.4 mmol) at 0° C. The mixture was stirred at 25° C. for 1 h and neutralized using NaHCO₃ (pH 7). Following dilution with water (50 mL), the product was extracted with EtOAc (50 mL×3). The combined organic layers were dried over Na₂SO₄, filtered, and concentrated under reduced pressure to give 5-bromo-7-fluoro-1-methyl-benzotriazole (3.11 g, 31% yield) as brown oil. LCMS (MM-ES+APCI, Pos): m/z 230.0 (M+H).

Step D. (7-fluoro-1-methyl-1H-benzo[d][1,2,3]triazol-5-yl)boronic acid. To a solution of 5-bromo-7-fluoro-1-methyl-benzotriazole (1.00 g, 4.35 mmol) in 1,4-dioxane (20 mL) was added 4,4,5,5-tetramethyl-2-(4,4,5,5-tetramethyl-1,3,2-dioxaborolan-2-yl)-1,3,2-dioxaborolane (1.66 g, 6.52 mmol), KOAc (1.28 g, 13.0 mmol), and Pd(dppf)Cl₂ (318 mg, 0.44 mmol). The mixture was stirred at 100° C. for 2 h under N₂. The reaction mixture was filtered, and the filtrate was concentrated under reduced pressure to give a residue which was purified by preparative HPLC (30% to 35% MeCN/0.2% aqueous FA). After lyophilization, (7-fluoro-1-methyl-benzotriazol-5-yl)boronic acid (100 mg, 9% yield) was obtained as a gray solid. LCMS (MM-ES+APCI, Pos): m/z 196.2 (M+H).

Step E. 7-fluoro-1-methyl-1H-benzo[d][1,2,3]triazol-5-ol. To a solution of (7-fluoro-1-methyl-benzotriazol-5-yl) boronic acid (100 mg, 0.51 mmol) in MeCN (1 mL) was added H₂O₂ (148 μL, 1.54 mmol, 30% wt.) at 0° C. and the mixture was stirred at 25° C. for 2 h. The reaction was quenched with aqueous sodium sulfite (30 mL) and extracted with EtOAc (30 mL×3). The combined organic layers were washed with brine (30 mL×3), dried over anhydrous Na₂SO₄, filtered, and concentrated under reduced pressure to afford 7-fluoro-1-methyl-benzotriazol-5-ol (170 mg, quant. yield) as a gray. LCMS (MM-ES+APCI, Pos): m/z 167.8 (M+H).

### Example 3 (I66)

### 1-(methyl-d3)-1H-benzo[d][1,2,3]triazol-5-ol

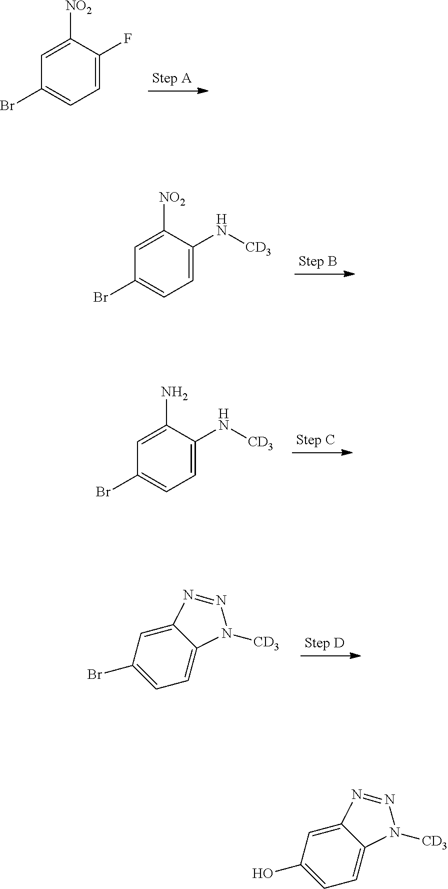 [SMILES: O=[N+]([O-])c1cc(Br)ccc1F ; [2H]C([2H])([2H])Nc1ccc(Br)cc1[N+](=O)[O-] ; [2H]C([2H])([2H])Nc1ccc(Br)cc1N ; [2H]C([2H])([2H])n1nnc2cc(Br)ccc21 ; [2H]C([2H])([2H])n1nnc2cc(O)ccc21]

Step A. 4-bromo-N-(methyl-d3)-2-nitroaniline. To a solution of 4-bromo-1-fluoro-2-nitrobenzene (195 g, 0.89 mol) and Cs₂CO₃ (289 g, 1.77 mol) in DMF (2 L) was added methyl-D3-amine hydrochloride (93.8 g, 1.33 mol). The reaction was heated to 80° C. for 1 h. The mixture was poured into water (6 L), stirred for 1 h, and filtered. The solid was collected and dried in vacuo to afford 4-bromo-N-(methyl-d3)-2-nitroaniline (203 g, 96% yield) as a red solid.

Step B. 4-bromo-N1-(methyl-d3)benzene-1,2-diamine. A solution of 4-bromo-N-(methyl-d3)-2-nitroaniline (170 g, 0.73 mol), iron (122 g, 2.18 mol), and NH₄Cl (117 g, 2.18 mol) in EtOH (1.7 L) and water (0.34 L) was heated to 80° C. for 3 h. The reaction was cooled to 30° C. and filtered over Celite, washing with EtOH (0.85 L). After concentrating in vacuo the mixture was basified (pH 9) with aq. NaOH (2M) and extracted with EtOAc (2×850 mL). The combined organics were dried over MgSO₄, filtered, and concentrated in vacuo to afford 4-bromo-N1-(methyl-d3)benzene-1,2-diamine (146 g, 85% yield).

Step C. 5-bromo-1-(methyl-d3)-1H-benzo[d][1,2,3]triazole. To a solution of 4-bromo-N1-(methyl-d3)benzene-1,2-diamine (23.7 g, 106 mmol) in water (237 mL) was added concentrated HCl (16 mL, 528 mmol). The reaction was cooled to 0° C. and a solution of NaNO₂ (10.9 g, 159 mmol) in water (237 mL) was added. The reaction was basified (pH 9) with aq. NaOH (2M) and extracted with DCM (3×75 mL). The combined organic layers were dried over MgSO₄, filtered, and concentrated in vacuo. The crude material was purified via silica gel column chromatography (10% to 35% EtOAc/PET) to afford 5-bromo-1-(methyl-d3)-1H-benzo[d][1,2,3]triazole (12.7 g, 35% yield) as a brown solid.

Step D. 1-(methyl-d3)-1H-benzo[d][1,2,3]triazol-5-ol. To a solution of Pd₂(dba)₃ (0.85 g, 0.93 mmol), tBuXPhos (0.79 g, 1.86 mmol), and KOH (2.97 g, 53.0 mmol) in 1,4-dioxane (40 mL) and water (8 mL) was added 5-bromo-1-(methyl-d3)-1H-benzo[d][1,2,3]triazole (2.00 g, 9.30 mmol). The reaction was heated to 85° C. for 2 h and cooled to ambient temperature. The mixture was acidified (pH 5) with aq. HCl and extracted with EtOAc (4×5 mL). The combined organic layers were dried over MgSO₄, filtered, and concentrated in vacuo. The crude material was purified via silica gel column chromatography (10% to 35% EtOAc/PET), then further purified via slurry (1:2 EtOAc:Heptanes) to afford 1-(methyl-d3)-1H-benzo[d][1,2,3]triazol-5-ol (200 mg, 12% yield) as a brown solid.

### Example 4 (I67)

### 7-methoxy-1-methyl-1H-benzo[d][1,2,3]triazol-5-ol

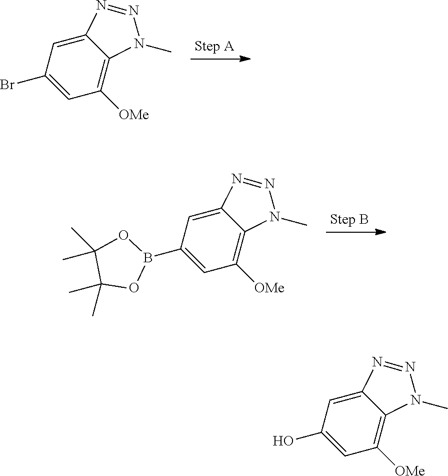

Step A. 5-bromo-7-methoxy-1-methyl-1H-benzo[d][1,2,3]triazole. To a solution of 5-bromo-7-methoxy-1-methyl-1H-benzo[d][1,2,3]triazole (1.00 g, 4.13 mmol), 4,4,5,5-tetramethyl-2-(4,4,5,5-tetramethyl-1,3,2-dioxaborolan-2-yl)-1,3,2-dioxaborolane (3.15 g, 12.4 mmol) and KOAc (1.22 g, 12.4 mmol) in 1,4-dioxane (20 mL) was added Pd(dppf)C₁₂ (302 mg, 0.41 mmol). The reaction was purged with N₂ and stirred for 2 h at 100° C. The mixture was filtered through celite eluting with EtOAc (50 mL). The filtrate was concentrated in vacuo and the residue was purified via silica gel column chromatography (0% to 100% EtOAc/PET) to provide 7-methoxy-1-methyl-5-(4,4,5,5-tetramethyl-1,3,2-dioxaborolan-2-yl)-1H-benzo[d][1,2,3]triazole (1.40 g, quant. yield) as an off-white solid. LCMS (MM-ES+APCI, Pos): m/z 290.2 (M+H).

Step B. 7-methoxy-1-methyl-1H-benzo[d][1,2,3]triazol-5-ol. To a solution of 7-methoxy-1-methyl-5-(4,4,5,5-tetramethyl-1,3,2-dioxaborolan-2-yl)-1H-benzo[d][1,2,3]triazole (1.30 g, 4.50 mmol) in THF (20 mL) and water (4 mL) was added NaBO₃ hydrate (1.73 mL 8.99 mmol). The mixture was stirred for 12 h at 25° C. The suspension was filtered, eluting with MeOH (150 mL). The filtrate was concentrated in vacuo and the residue was triturated by slurry in EtOAc (15 mL) and water (15 mL) at 25° C. for 0.5 h. After filtration, the cake was dried under vacuum to give 7-methoxy-1-methyl-1H-benzo[d][1,2,3]triazol-5-ol 3 (1.60 g, quant. yield) as an off-white solid. LCMS (MM-ES+APCI, Pos): m/z 180.1 (M+H).

### Example 5 (168)

### 6-fluoro-1-methyl-H-benzo[d][1,2,3]triazol-5-ol

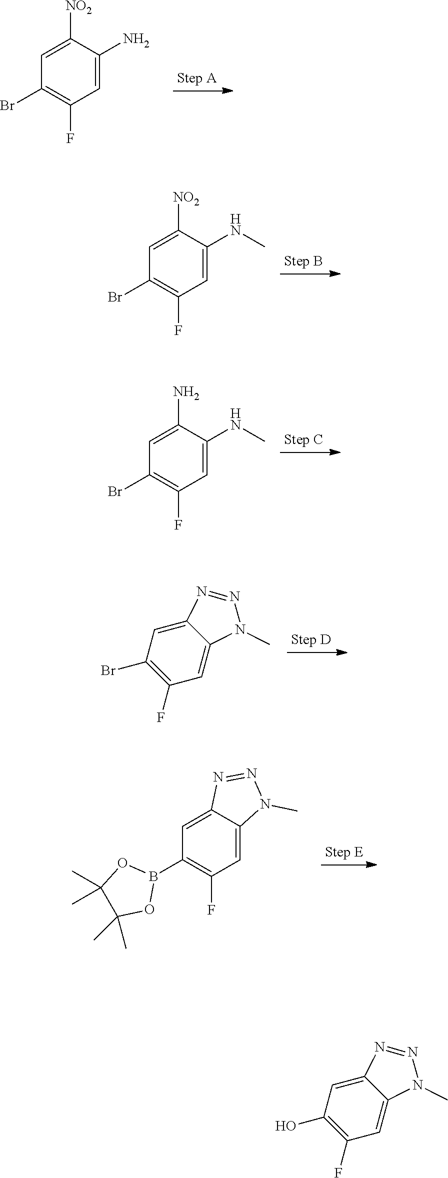

Steps A to C. 5-bromo-6-fluoro-1-methyl-H-benzo[d][1,2,3]triazole. Prepared according to Example 3 substituting 4-bromo-5-fluoro-2-nitroaniline for 4-bromo-2-fluoro-6-nitro-aniline to afford 5-bromo-6-fluoro-1-methyl-1H-benzo[d][1,2,3]triazole (580 mg, 51% yield over 3 steps) as a brown solid. LCMS (MM-ES+APCI, Pos): m/z 229.8 (M+H).

Steps D to E. 6-fluoro-1-methyl-1H-benzo[d][1,2,3]triazol-5-ol. Prepared according to Example 4 steps A to B, substituting for 5-bromo-6-fluoro-1-methyl-1H-benzo[d][1,2,3]triazole for 5-bromo-7-methoxy-1-methyl-H-benzo[d][1,2,3]triazole to afford 6-fluoro-1-methyl-1H-benzo[d][1,2,3]triazol-5-ol (450 mg, 88% yield over 2 steps) as a gray solid. LCMS (MM-ES+APCI, Pos): m/z 168.1 (M+H).

Intermediates 168 to 171 in Table 11 below were prepared according to Example 5 Steps A to E.

TABLE 11
A list of intermediate compounds I68-I71

| | | | MS |
|---|---|---|---|
| | | | API |
| Int. ID | Structure | Chemical Name | (m/z) |
| I68 | 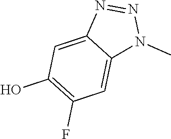 | 6-fluoro-1-methyl-1H-benzo [d][1,2,3]triazol-5-ol | 168.1 (M + H) |
| I69 | 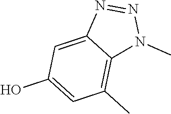 | 1,7-dimethyl-1H-benzo[d] [1,2,3]triazol-5-ol | 164.2 (M + H) |
| I70 | 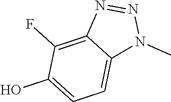 | 4-fluoro-1-methyl-1H-benzo [d][1,2,3]triazol-5-ol | 167.9 (M + H) |
| I71 | 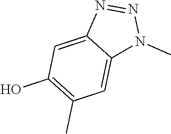 | 1,6-dimethyl-1H-benzo[d] [1,2,3]triazol-5-ol | 164.3 (M + H) |

### Example 6 (I72)

### 7-fluoro-1,6-dimethyl-1H-benzo[d][1,2,3]triazol-5-ol

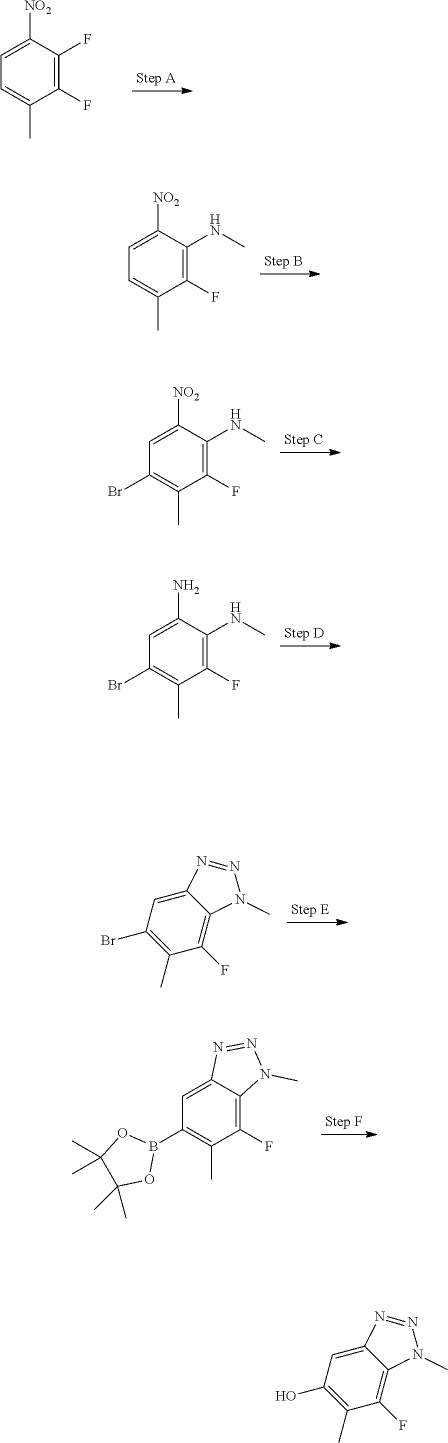

Step A. 2-fluoro-N,3-dimethyl-6-nitroaniline. To a solution of 2,3-difluoro-1-methyl-4-nitro-benzene (10.0 g, 57.8 mmol) and methanamine hydrochloride (5.85 g, 86.7 mmol) in DMF (130 mL) was added K₂CO₃ (7.98 g, 57.8 mmol). The mixture was stirred for 2 h at 25° C. The reaction was poured into water (300 mL) and the resulting precipitate was filtered, rinsing with water (250 mL). The solid was dried in vacuo, the triturated in 10% EtOAc/PET (110 mL) for 10 min. Filtration and drying afforded 2-fluoro-N,3-dimethyl-6-nitro-aniline (5.6 g, 52% yield) as a yellow solid. LCMS (MM-ES+APCI, Pos): m/z 185.1 (M+H).

Step B. 4-bromo-2-fluoro-N,3-dimethyl-6-nitroaniline. To a solution of 2-fluoro-N,3-dimethyl-6-nitro-aniline (5.6 g, 30.1 mmol) in MeCN (50 mL) was added NBS (5.36 g, 30.1 mmol). The mixture stirred for 2 h at 25° C. The reaction was quenched with water (30 mL) and the product extracted with DCM (40 mL×3). The combined organic layers were washed with brine (30 mL), dried over anhydrous sodium sulfate, filtered, and concentrated under reduced pressure to afford 4-bromo-2-fluoro-N,3-dimethyl-6-nitro-aniline (5.1 g, 64% yield) as a red solid. LCMS (MM-ES+APCI, Pos): m/z 263.0, 265.0 (M+H).

Step C to Step F. 7-fluoro-1,6-dimethyl-1H-benzo[d][1,2,3]triazol-5-ol. Prepared according to Example 4 steps B to E, substituting 4-bromo-2-fluoro-N,3-dimethyl-6-nitroaniline for 4-bromo-5-fluoro-2-nitroaniline to afford 7-fluoro-1,6-dimethyl-1H-benzo[d][1,2,3]triazol-5-ol (765 mg, 74% yield) as a brown solid. LCMS (MM-ES+APCI, Pos): m/z 182.3 (M+H).

### Example 7 (I73)

### 6-fluoro-1,7-dimethyl-1H-benzo[d][1,2,3]triazol-5-ol

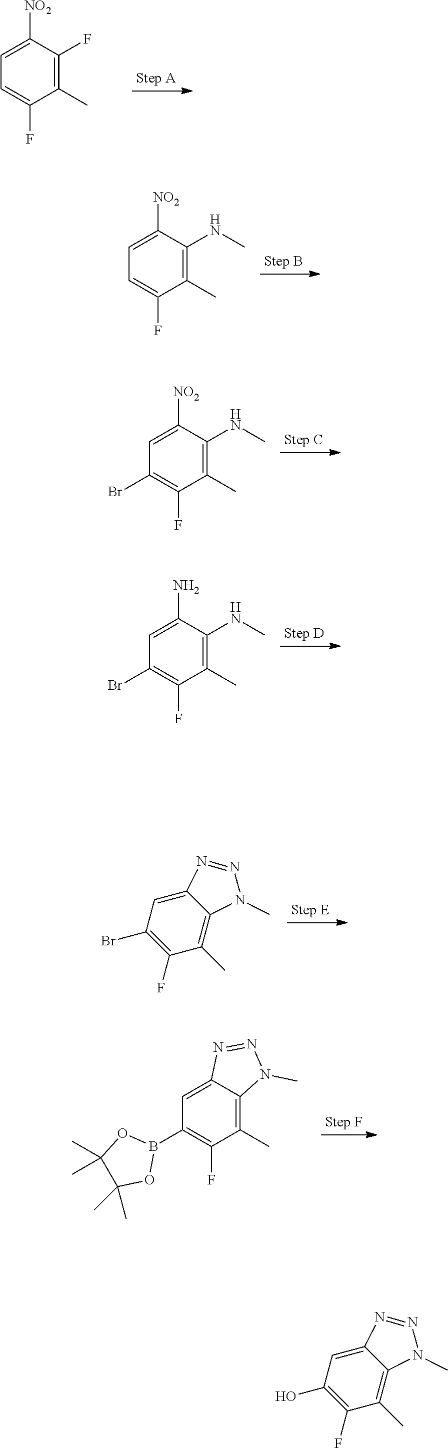

Steps A to F. 6-fluoro-1,7-dimethyl-1H-benzo[d][1,2,3]triazol-5-ol. Prepared according to Example 6 steps A to F, substituting 1,3-difluoro-2-methyl-4-nitrobenzene for 2,3-difluoro-1-methyl-4-nitro-benzene to afford 6-fluoro-1,7-dimethyl-1H-benzo[d][1,2,3]triazol-5-ol (2.2 g, 52% yield) as a brown solid. LCMS (MM-ES+APCI, Pos): m/z 182.0 (M+H).

### Example 8 (I74)

### 4-((3,5-dimethyl-3H-imidazo[4,5-b]pyridin-6-yl)oxy)-3-methylaniline

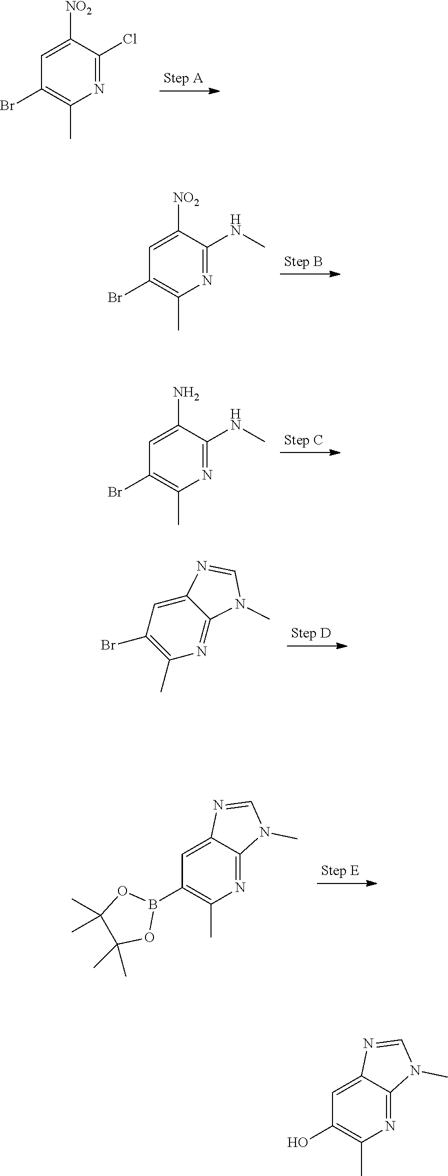

Step A. 5-bromo-N,6-dimethyl-3-nitropyridin-2-amine. To a solution of 5-bromo-2-chloro-6-methyl-3-nitro-pyridine (4.90 g, 19.5 mmol) and methanamine hydrochloride (2.63 g, 39.0 mmol) in acetonitrile (70 mL) was added DIPEA (13.6 mL, 77.9 mmol). The mixture was stirred at 50° C. for 12 h. The reaction was quenched with water and extracted with EtOAc (200 mL×3). The combined organic layers were washed with brine (200 mL), dried over anhydrous sodium sulfate, filtered, and concentrated under reduced pressure to afford 5-bromo-N,6-dimethyl-3-nitro-pyridin-2-amine (5.37 g, quant. yield) as a yellow solid. LCMS (MM-ES+APCI, Pos): m/z 245.8, 247.8 (M+H).

Step B. 5-bromo-N2,6-dimethylpyridine-2,3-diamine. To a solution of 5-bromo-N,6-dimethyl-3-nitro-pyridin-2-amine (5.37 g, 21.8 mmol) in EtOH (60 mL) and water (20 mL) were added NH₄Cl (11.6 g, 218 mmol) and Fe powder (8.53 g, 153 mmol). The mixture was stirred at 80° C. for 2 h. The reaction was filtered and the filtrate was concentrated under reduced pressure. The residue was partitioned between sat. aq. NaHCO₃ (200 mL) and EtOAc (200 mL×3). The combined organic layers were washed with brine (100 mL), dried over anhydrous sodium sulfate, filtered, and concentrated under reduced pressure to afford 5-bromo-N2,6-dimethyl-pyridine-2,3-diamine (4.48 g, 95% yield) as a yellow solid. LCMS (MM-ES+APCI, Pos): m/z 216.0, 218.0 (M+H).

Step C. 6-bromo-3,5-dimethyl-3H-imidazo[4,5-b]pyridine. A solution of 5-bromo-N2,6-dimethyl-pyridine-2,3-diamine 3 (4.48 g, 20.7 mmol) in trimethoxymethane (50 mL) was stirred at 110° C. for 1 h. The mixture was concentrated under reduced pressure and the residue was partitioned between sat. aq. NaHCO₃ (100 mL) and EtOAc (100 mL×3). The combined organic layers were washed with brine (100 mL), dried over anhydrous sodium sulfate, filtered, and concentrated in vacuo. The residue was purified by silica gel column chromatography (0% to 100% EtOAc/PET) to afford 6-bromo-3,5-dimethyl-imidazo[4,5-b]pyridine (3.72 g, 79% yield) as a pink solid. LCMS (MM-ES+APCI, Pos): m/z 226.1 (M+H).

Step D to E. 3,5-dimethyl-3H-imidazo[4,5-b]pyridin-6-ol. Prepared according to Example 4 steps D to E, substituting 6-bromo-3,5-dimethyl-3H-imidazo[4,5-b]pyridine for 5-bromo-6-fluoro-1-methyl-1H-benzo[d][1,2,3]triazole to afford 3,5-dimethyl-3H-imidazo[4,5-b]pyridin-6-ol (2.9 g, 72% yield) as an off-white solid. LCMS (MM-ES+APCI, Pos): m/z 164.0 (M+H).

### Example 9 (I75)

### 3-methyl-3H-[1,2,3]triazolo[4,5-c]pyridin-6-ol

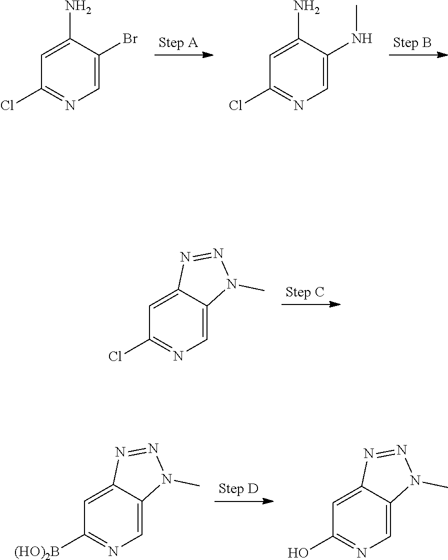

Step A. 6-chloro-N3-methylpyridine-3,4-diamine. To a solution of 5-bromo-2-chloro-pyridin-4-amine (2.00 g, 9.64 mmol), methanamine hydrochloride (6.51 g, 96.4 mmol) and 1-(2-pyridyl)ethanone oxime (0.53 g, 3.86 mmol) in 1-methylpyrrolidin-2-one (30 mL) were added K₂CO₃ (6.66 g, 48.2 mmol) and CuI (0.37 g, 1.93 mmol). The reaction was purged with N₂ and heated at 130° C. for 12 h in a sealed tube. The mixture was cooled to ambient temperature, diluted with water (200 mL) and extracted with EtOAc (200 mL×3). The combined organic layers were washed with brine (200 mL×3), dried over anhydrous sodium sulfate, filtered and concentrated under reduced pressure. The crude residue was purified by silica gel column chromatography (0% to 15% EtOAc/PET) too afford 6-chloro-N3-methyl-pyridine-3,4-diamine (1.70 g, 38% yield) as a yellow oil. LCMS (MM-ES+APCI, Pos): m/z 158.1 (M+H).

Step B. 6-chloro-3-methyl-3H-[1,2,3]triazolo[4,5-c]pyridine. A flask containing 6-chloro-N3-methyl-pyridine-3,4-diamine (1.70 g, 10.8 mmol) was placed under N₂ and cooled to 0° C. A solution of H₂SO₄ (1.61 mL, 30.2 mmol) in water (30 mL) was added, followed by NaNO₂ (1.49 g, 21.6 mmol) in water (20 mL). The mixture was stirred at 0° C. for 1 h. The reaction was neutralized with sat. aq. NH₄HCO₃ (pH=7) and extracted with EtOAc (50 mL×3). The combined organic layers were washed with brine (50 mL), dried over anhydrous sodium sulfate, filtered and concentrated in vacuo. The crude residue was purified by silica gel column chromatography (0% to 50% EtOAc/PET) to afford 6-chloro-3-methyl-triazolo[4,5-c]pyridine (530 mg, 27% yield) as a red solid. LCMS (MM-ES+APCI, Pos): m/z 169.2 (M+H).

Step C. (3-methyl-3H-[1,2,3]triazolo[4,5-c]pyridin-6-yl)boronic acid. To a solution of 6-chloro-3-methyl-triazolo[4,5-c]pyridine 4 (530 mg, 3.14 mmol) and 4,4,5,5-tetramethyl-2-(4,4,5,5-tetramethyl-1,3,2-dioxaborolan-2-yl)-1,3,2-dioxaborolane (2.40 g, 9.43 mmol) in 1,4-dioxane (10 mL) were added KOAc (617 mg, 6.29 mmol), SPhos (258 mg, 0.63 mmol) and Pd₂(dba)₃ (287 mg, 0.31 mmol). The reaction was purged with N₂ and heated to 100° C. for 12 h. The mixture was concentrated in vacuo to provide (3-methyltriazolo[4,5-c]pyridin-6-yl)boronic acid (500 mg, 89% yield). LCMS (MM-ES+APCI, Pos): m/z 178.9 (M+H).

Step D. 3-methyl-3H-[1,2,3]triazolo[4,5-c]pyridin-6-ol. To a solution of (3-methyltriazolo[4,5-c]pyridin-6-yl)boronic acid (500 mg, 2.81 mmol) in THF (10 mL) and water (5 mL) was added sodium borate tetrahydrate (1.08 mL, 5.62 mmol). The reaction was stirred at 50° C. for 1 h. The mixture was filtered and the filtrate concentrated in vacuo. The residue was purified via preparative HPLC (0.1% aqueous NH₄OH) and lyophilized to afford 3-methyltriazolo[4,5-c]pyridin-6-ol 6 (400 mg, 95% yield) as a yellow solid. LCMS (MM-ES+APCI, Pos): m/z 152.1 (M+H).

### Example 10 (I3)

### 4-((7-fluoro-1-methyl-1H-benzo[d]imidazol-5-yl)oxy)-3-methylaniline

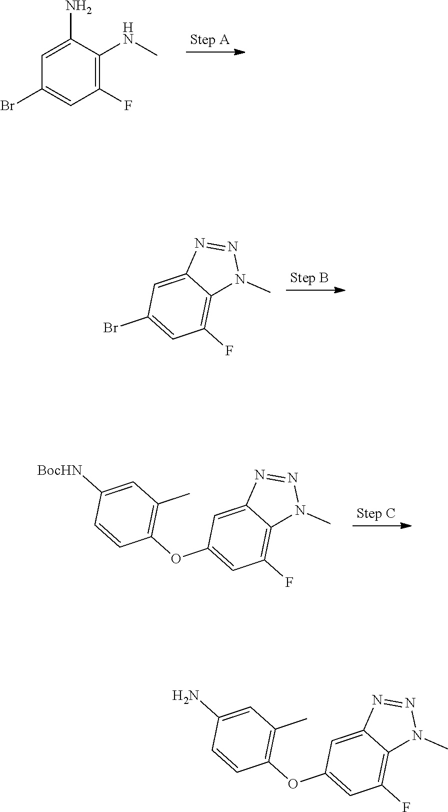 [SMILES: CNc1c(N)cc(Br)cc1F ; Cn1nnc2cc(Br)cc(F)c21 ; Cc1cc(NC(=O)OC(C)(C)C)ccc1Oc1cc(F)c2c(c1)nnn2C ; Cc1cc(N)ccc1Oc1cc(F)c2c(c1)nnn2C]

Step A. 5-bromo-7-fluoro-1-methyl-1H-benzo[d]imidazole formate. To a solution of 4-bromo-6-fluoro-N¹-methylbenzene-1,2-diamine (2.40 g, 10.9 mmol) in formic acid (20 mL) was added trimethoxymethane (1.20 mL, 10.9 mmol). The mixture was stirred at 100° C. for 1 h and concentrated in vacuo to afford 5-bromo-7-fluoro-1-methyl-1H-benzo[d]imidazole formate (2.00 g, 67%) as a yellow oil. LCMS (MM-ES+APCI, Pos): m/z 228.7, 230.7 (M+H-FA).

Step B. tert-butyl (4-((7-fluoro-1-methyl-1H-benzo[d]imidazol-5-yl)oxy)-3-methylphenyl)carbamate. To a solution of 5-bromo-7-fluoro-1-methyl-1H-benzo[d]imidazole formate (2.00 g, 8.73 mmol) and tert-butyl N-(4-hydroxy-3-methyl-phenyl)carbamate (1.95 g, 8.73 mmol) in DMSO (20 mL) was added CuI (0.67 g, 3.49 mmol), pyridine-2-carboxylic acid (0.43 g, 3.49 mmol) and K₂CO₃ (3.62 g, 26.2 mmol). The reaction was stirred under N₂ at 100° C. for 12 h. The mixture was filtered, concentrated under reduced pressure, and purified by preparative HPLC (30% to 65% MeCN/0.05% aqueous FA) to yield tert-butyl (4-((7-fluoro-1-methyl-1H-benzo[d]imidazol-5-yl)oxy)-3-methylphenyl)carbamate (1.40 g, 40% yield) as a black-brown solid. LCMS (MM-ES+APCI, Pos): m/z 372.1 (M+H).

Step C. 4-((7-fluoro-1-methyl-1H-benzo[d]imidazol-5-yl)oxy)-3-methylaniline hydrochloride. To a solution of tert-butyl (4-((7-fluoro-1-methyl-1H-benzo[d]imidazol-5-yl)oxy)-3-methylphenyl)carbamate (1.40 g, 3.77 mmol) in MeOH (5 mL) was added HCl (4M in 1,4-dioxane, 20.3 mL, 81.2 mmol). The mixture was stirred for 2 h and concentrated in vacuo to afford 4-(7-fluoro-1-methyl-benzimidazol-5-yl)oxy-3-methyl-aniline hydrochloride (1.00 g, 86% yield) as a black brown solid. LCMS (MM-ES+APCI, Pos): m/z 272.0 (M+H—HCl).

### Example 11 (I76)

### 7-fluoro-1-methyl-1H-benzo[d]imidazol-5-ol

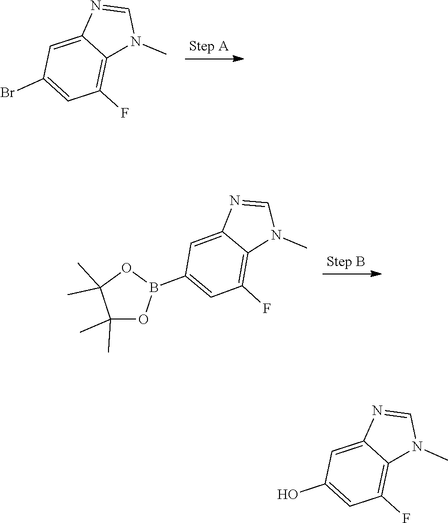

Step A. 7-fluoro-1-methyl-5-(4,4,5,5-tetramethyl-1,3,2-dioxaborolan-2-yl)-1H-benzo[d]imidazole. To a flask containing 5-bromo-7-fluoro-1-methyl-benzimidazole (7.0 g, 29 mmol), 4,4,5,5-tetramethyl-2-(4,4,5,5-tetramethyl-1,3,2-dioxaborolan-2-yl)-1,3,2-dioxaborolane (11 g, 44 mmol), KOAc (5.8 g, 59 mmol) and Pd(dppf)Cl₂ (1.1 g, 1.5 mmol) was added 1,4-dioxane (200 mL) at 25° C. The mixture was purged with N₂ and heated to 90° C. for 2 h. The reaction was quenched slowly with water (200 mL), and the aqueous layer mixture was extracted with EtOAc (100 mL×3). The combined organic layers were washed with brine (150 mL), dried over anhydrous sodium sulfate, filtered, and concentrated under reduced pressure. The crude residue was purified by silica gel column chromatography (0% to 20% EtOAc/PET) to afford 7-fluoro-1-methyl-5-(4,4,5,5-tetramethyl-1,3,2-dioxaborolan-2-yl) benzimidazole (7.6 g, 74% yield) as a brown solid. LCMS (MM-ES+APCI, Pos): m/z 276.8 (M+H).

Step B. 7-fluoro-1-methyl-1H-benzo[d]imidazol-5-ol. To a solution of 7-fluoro-1-methyl-5-(4,4,5,5-tetramethyl-1,3,2-dioxaborolan-2-yl) benzimidazole (7.3 g, 21 mmol) in MeCN (60 mL) and water (60 mL) was added Oxone (6.4 g, 10 mmol). The mixture was heated to 50° C. for 3 h. The reaction was quenched slowly with water (200 mL) and the aqueous layer mixture was extracted with EtOAc (100 mL×3). The combined organic layers were washed with sat. aq. Na₂SO₃ (300 mL) and brine (200 mL), dried over anhydrous sodium sulfate, filtered, and concentrated under reduced pressure. The crude product was purified by silica gel column chromatography (0% to 5% MeOH/DCM) to afford 7-fluoro-1-methyl-benzimidazol-5-ol (3.2 g, 85% yield) as a gray solid. LCMS (MM-ES+APCI, Pos): m/z 167.0 (M+H).

### Example 12 (I4)

### 1-(difluoromethyl)-1H-benzo[d][1,2,3]triazol-5-ol

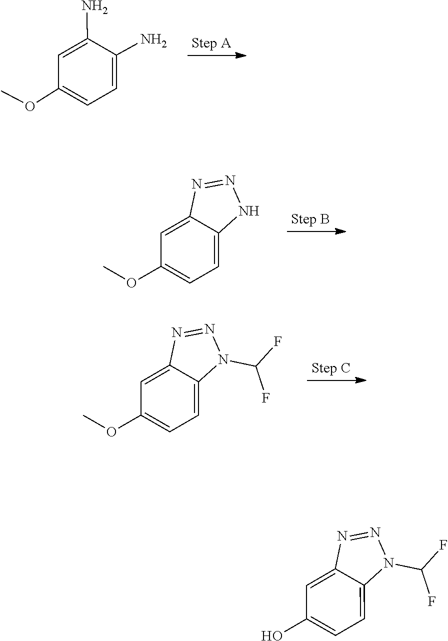

Step A. 5-methoxy-1H-benzo[d][1,2,3]triazole. Prepared according to Example 1 Step C, substituting 4-methoxybenzene-1,2-diamine for 4-methoxy-N¹-methyl-benzene-1,2-diamine to afford 5-methoxy-1H-benzotriazole (5.03 g, 48% yield) as a yellow solid. LCMS (MM-ES+APCI, Pos): m/z 150.3 (M+H).

Step B. 1-(difluoromethyl)-5-methoxy-1H-benzo[d][1,2,3]triazole. To a solution of 5-methoxy-1H-benzotriazole (5.27 g, 35.3 mmol) in DMF (50 mL) was added sodium chlorodifluoroacetate (10.8 g, 70.7 mmol) and Cs₂CO₃ (23.0 g, 70.7 mmol). The reaction mixture was stirred at 80° C. for 1 h, diluted with water (400 mL) and extracted with EtOAc (100 mL×4). The combined organic layers were washed with brine (200 mL×2), dried over anhydrous Na₂SO₄, filtered, and concentrated under reduced pressure to afford 1-(difluoromethyl)-5-methoxy-benzotriazole (6.49 g, 73% yield) as a yellow solid. LCMS (MM-ES+APCI, Pos): m/z 200.1 (M+H).

Step C. 1-(difluoromethyl)-1H-benzo[d][1,2,3]triazol-5-ol. Prepared according to Example 1 Step D, substituting 1-(difluoromethyl)-5-methoxy-benzotriazole for 5-methoxy-1-methyl-benzotriazole to provide 1-(difluoromethyl) benzotriazol-5-ol (6.12 g, 89% yield) as a brown solid. LCMS (MM-ES+APCI, Pos): m/z 186.2 (M+H).

### Example 13 (I5)

### 1-(difluoromethyl)-1H-benzo[d]imidazol-5-ol

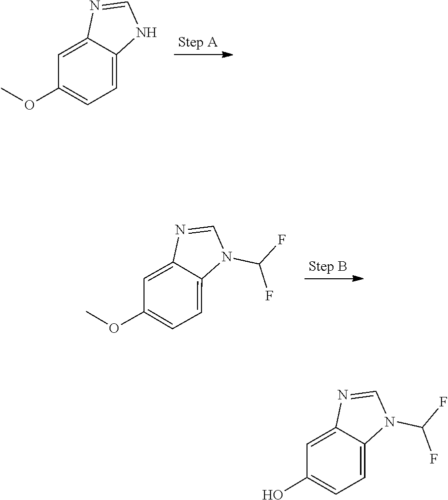

1-(difluoromethyl)-1H-benzo[d]imidazol-5-ol. Prepared according to Example 12 Steps B to C, substituting 5-methoxy-1H-benzotriazole for 5-methoxy-1H-benzo[d]imidazole (3.00 g, 20.25 mmol) to afford 1-(difluoromethyl)benzimidazol-5-ol (2.04 g, 81% yield) as a white solid. LCMS (MM-ES+APCI, Pos): m/z 185.1 (M+H).

### Example 14 (I77)

### 1-(trifluoromethyl)-1H-benzo[d][1,2,3]triazol-5-ol

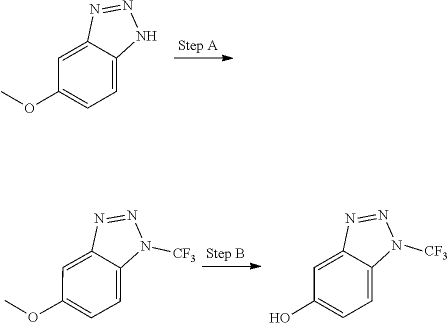

Step A. 5-methoxy-1-(trifluoromethyl)-1H-benzo[d][1,2,3]triazole. Two parallel batches each contained a solution of 5-methoxy-1H-benzotriazole (250 mg, 1.68 mmol), 3,3-dimethyl-1-(trifluoromethyl)-1,2-benziodoxole (830 mg, 2.51 mmol), and 1,1,1-trifluoro-N-(trifluoromethylsulfonyl)methanesulfonamide (47.1 mg, 0.17 mmol) in CS₂ (10 mL). The mixture was stirred at 60° C. for 1 h and concentrated in vacuo. The two batches were combined and purified by silica gel column chromatography (0% to 20% EtOAc in PET) to afford 5-methoxy-1-(trifluoromethyl)benzotriazole (120 mg, 15% yield) as a light yellow solid. LCMS (MM-ES+APCI, Pos): m/z 218.0 (M+H).

Step B. 1-(trifluoromethyl)-1H-benzo[d][1,2,3]triazol-5-ol. Prepared according to Example 1 Step D, substituting 5-methoxy-1-(trifluoromethyl)-1H-benzo[d][1,2,3]triazole for 5-methoxy-1-methyl-benzotriazole to provide 1-(trifluoromethyl)-1H-benzo[d][1,2,3]triazol-5-ol (150 mg, 67% yield) as a pale yellow solid. LCMS (MM-ES+APCI, Pos): m/z 204.0 (M+H).

### Example 15 (I78)

### 3-(difluoromethyl)-3H-imidazo[4,5-b]pyridin-6-ol

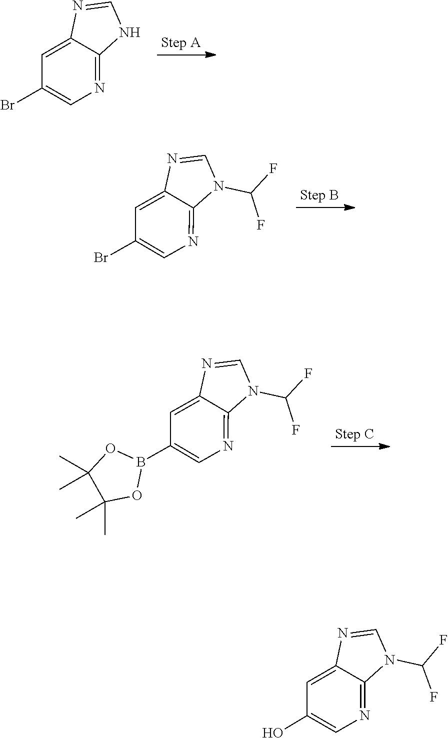 [SMILES: Brc1cnc2[nH]cnc2c1 ; FC(F)n1cnc2cc(Br)cnc21 ; CC1(C)OB(c2cnc3c(c2)ncn3C(F)F)OC1(C)C ; Oc1cnc2c(c1)ncn2C(F)F]

Step A. 6-bromo-3-(difluoromethyl)-3H-imidazo[4,5-b]pyridine. To a solution of 6-bromo-3H-imidazo[4,5-b]pyridine (22.0 g, 111 mmol) and sodium 2-chloro-2,2-difluoroacetate (25.4 g, 167 mmol) in DMF (300 mL) was added Cs₂CO₃ (72.4 g, 222 mmol). The reaction was stirred at 80° C. for 2 h. The mixture was partitioned between EtOAc (500 mL) and water (500 mL). The aqueous phase was extracted with EtOAc (500 mL×2), and the combined organic layers were washed with brine (500 mL×2), dried over sodium sulfate, filtered, and concentrated under vacuum. The residue was purified by silica gel column chromatography (0% to 100% EtOAc/PET) to afford 6-bromo-3-(difluoromethyl)-3H-imidazo[4,5-b]pyridine (3.00 g, 11% yield) as a light yellow solid. LCMS (MM-ES+APCI, Pos): m/z 247.9 (M+H).

Steps B to C. 3-(difluoromethyl)-3H-imidazo[4,5-b]pyridin-6-ol. Prepared according to Example 4 steps A to B, substituting 6-bromo-3-(difluoromethyl)-3H-imidazo[4,5-b]pyridine for 5-bromo-7-methoxy-1-methyl-H-benzo[d][1,2,3]triazole to afford 3-(difluoromethyl)-3H-imidazo[4,5-b]pyridin-6-ol (1.63 g, 67% yield) as a white solid. LCMS (MM-ES+APCI, Pos): m/z 186.2 (M+H).

### Example 16 (I79)

### 2-methyl-[1,2,4]triazolo[1,5-a]pyridin-7-ol

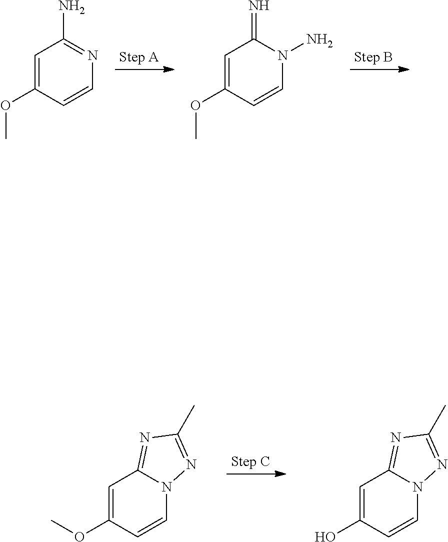

Step A. 2-imino-4-methoxypyridin-1(2H)-amine. To a solution of 4-methoxypyridin-2-amine (2.00 g, 16.1 mmol) in DCM (30 mL) was added O-(mesitylsulfonyl) hydroxylamine trifluoroacetate (8.30 g, 25.2 mmol). The mixture was stirred at 25° C. for 12 h. The reaction was filtered, and the filter cake was dried under vacuum to afford 2-imino-4-methoxypyridin-1(2H)-amine (2.00 g, 89% yield) as a white solid. LCMS (MM-ES+APCI, Pos): m/z 140.2 (M+H).

Step B. 7-methoxy-2-methyl-[1,2,4]triazolo[1,5-a]pyridine. To a solution of 2-imino-4-methoxypyridin-1(2H)-amine (2.00 g, 14.4 mmol) in pyridine (20 mL) was added acetyl chloride (2.04 mL, 28.7 mmol). The mixture was stirred at 100° C. for 12 h. The reaction was cooled to ambient temperature, diluted with water (100 mL), and extracted with EtOAc (50 mL×3). The combined organic layers were washed with brine (50 mL×2), dried over anhydrous sodium sulfate, filtered and concentrated in vacuo. The crude residue was purified by silica gel column chromatography (0% to 35% EtOAc/PET) to afford 7-methoxy-2-methyl-[1,2,4]triazolo[1,5-a]pyridine (0.73 g, 31% yield) as a light yellow solid. LCMS (MM-ES+APCI, Pos): m/z 164.2 (M+H).

Step C. 2-methyl-[1,2,4]triazolo[1,5-a]pyridin-7-ol. A sealed tube containing 7-methoxy-2-methyl-[1,2,4]triazolo[1,5-a]pyridine (930 mg, 5.70 mmol) and HBr (33% in AcOH, 10 mL) was stirred at 100° C. for 24 h. The mixture was concentrated under vacuum and the residue was diluted with sat. aq. NaHCO₃ (100 mL). The aqueous mixture was extracted with EtOAc (30 mL×3), and the aqueous phase was lyophilized to afford 2-methyl-[1,2,4]triazolo[1,5-a]pyridin-7-ol (610 mg, 72% yield) as a yellow solid. LCMS (MM-ES+APCI, Pos): m/z 150.2 (M+H).

### Example 17 (I80)

### 6-methyl-[1,2,4]triazolo[1,5-a]pyridin-7-ol

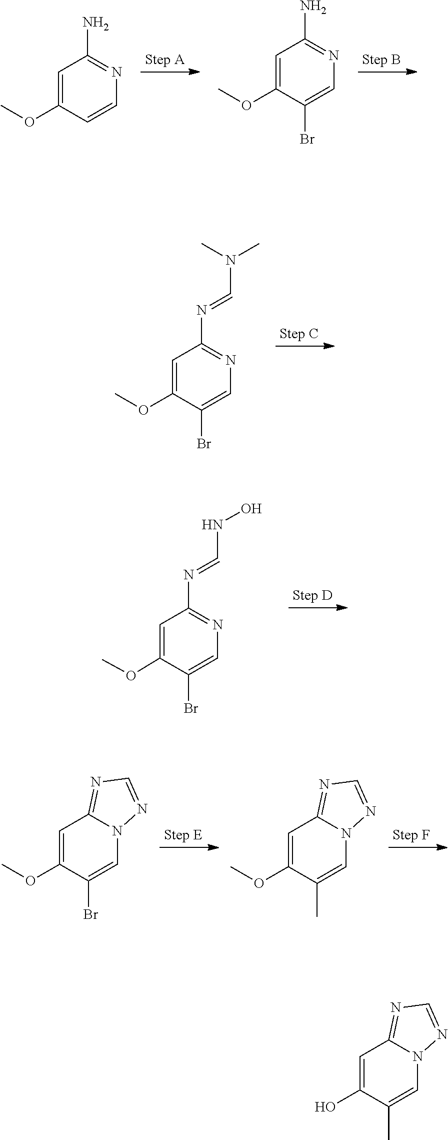

Step A. 5-bromo-4-methoxypyridin-2-amine. To a solution of 4-methoxypyridin-2-amine (15.0 g, 121 mmol) in MeCN (400 mL) was added NBS (21.5 g, 121 mmol). The reaction was stirred at 20° C. for 3 h. The mixture was concentrated in vacuo and the residue was partitioned between H₂O (100 mL) and EtOAc (100 mL×3). The combined organic layers were washed with brine (100 mL), dried over anhydrous sodium sulfate, filtered, and concentrated under reduced pressure to afford 5-bromo-4-methoxy-pyridin-2-amine (21.0 g, 85% yield) was obtained as a brown solid. LCMS (MM-ES+APCI, Pos): m/z 203.2, 205.2 (M+H).

Step B. (E)-N′-(5-bromo-4-methoxypyridin-2-yl)-N,N-dimethylformimidamide. To a solution of 5-bromo-4-methoxy-pyridin-2-amine (20.0 g, 98.5 mmol) in MeOH (200 mL) was added DMF-DMA (15.7 mL, 1118 mmol). The reaction was stirred at 75° C. for 3 h. The mixture was concentrated under reduced pressure to afford N′-(5-bromo-4-methoxy-2-pyridyl)-N,N-dimethyl-formamidine (21.0 g, 83% yield) as a brown oil. LCMS (MM-ES+APCI, Pos): m/z 258.2, 260.2 (M+H).

Step C. (E)-N′-(5-bromo-4-methoxypyridin-2-yl)-N-hydroxyformimidamide. To a solution of N′-(5-bromo-4-methoxy-2-pyridyl)-N,N-dimethyl-formamidine (21.0 g, 81.4 mmol) in MeOH (220 mL) was added hydroxylamine hydrochloride (11.3 g, 163 mmol). The reaction was stirred at 70° C. for 1 h. The mixture was filtered and the filter cake was washed with MeOH (20 mL). The filter cake was dried under vacuum to give N′-(5-bromo-4-methoxy-2-pyridyl)-N-hydroxy-formamidine (17.8 g, 88% yield) as a yellow solid. LCMS (MM-ES+APCI, Pos): m/z 246.2, 248.2 (M+H).

Step D. 6-bromo-7-methoxy-[1,2,4]triazolo[1,5-a]pyridine. To a solution of N′-(5-bromo-4-methoxy-2-pyridyl)-N-hydroxy-formamidine (17.8 g, 71.3 mmol) in THF (200 mL) was added trifluoroacetic anhydride (12.9 mL, 92.7 mmol). The mixture was stirred at 20° C. for 12 h. The reaction was quenched with water (50 mL) and extracted with EtOAc (30 mL×3). The combined organic layers were washed with brine (60 mL), dried over anhydrous sodium sulfate, filtered, and concentrated under reduced pressure. The crude residue was purified by silica gel column chromatography (0% to 65% EtOAc/PET) to provide 6-bromo-7-methoxy-[1,2,4]triazolo[1,5-a]pyridine (3.0 g, 18% yield) as a yellow solid. LCMS (MM-ES+APCI, Pos): m/z 228.2, 230.2 (M+H).

Step E. 7-methoxy-6-methyl-[1,2,4]triazolo[1,5-a]pyridine. To a solution of 6-bromo-7-methoxy-[1,2,4]triazolo[1,5-a]pyridine (3.0 g, 13.2 mmol) in 1,4-dioxane (30 mL) and water (6 mL) followed by the addition of dioxane (30 mL) and H2O (6 mL) were added Pd(dppf)Cl₂ (0.96 g, 1.32 mmol), K₂CO₃ (5.45 g, 39.5 mmol) and methylboronic acid (1.57 g, 26.3 mmol). The mixture was purged with N₂ and stirred at 110° C. for 2 h. The reaction was quenched with water (20 mL) and extracted with EtOAc (30 mL×3). The combined organic layers were washed with brine (30 mL), dried over anhydrous sodium sulfate, filtered, and concentrated under reduced pressure. The crude residue was purified by silica gel column chromatography (0% to 65% EtOAc/PET) to provide 7-methoxy-6-methyl-[1,2,4]triazolo[1,5-a]pyridine (900 mg, 27% yield) as a yellow solid. LCMS (MM-ES+APCI, Pos): m/z 164.4 (M+H).

Step F. 6-methyl-[1,2,4]triazolo[1,5-a]pyridin-7-ol. To a solution of 7-methoxy-6-methyl-[1,2,4]triazolo[1,5-a]pyridine (850 mg, 3.33 mmol) was added pyridine hydrochloride (7.71 g, 66.7 mmol). The mixture was purged with N₂ and heated to 145° C. for 2 h. The reaction was cooled to ambient temperature, quenched with sat. aq. NH₄Cl (20 mL) and extracted with EtOAc (30 mL×3). The combined organic layers were washed with brine (60 mL), dried over anhydrous sodium sulfate, filtered, and concentrated under reduced pressure to afford 6-methyl-[1,2,4]triazolo[1,5-a]pyridin-7-ol (380 mg, 77% yield) as a white solid. LCMS (MM-ES+APCI, Pos): m/z 150.4 (M+H).

### Example 18 (I81)

### imidazo[1,2-b]pyridazin-7-ol

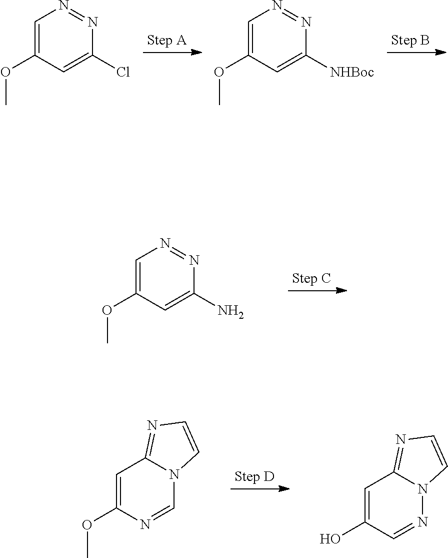

Step A. tert-butyl (5-methoxypyridazin-3-yl)carbamate. Two batches in parallel contained 3-chloro-5-methoxy-pyridazine (5.0 g, 35 mmol) and tert-butyl carbamate (8.10 g, 69 mmol) in 1,4-dioxane (100 mL). Xantphos (2.0 g, 3.5 mmol), Cs₂CO₃ (22.5 g, 69 mmol), and Pd(OAc)₂ (0.78 g, 3.5 mmol) were added and the mixture was purged with N₂. The reaction was heated at 90° C. for 5 h. The mixture was diluted with water (100 mL) and extracted with EtOAc (150 mL×3). The combined organic layers were washed with brine (150 mL), dried over anhydrous sodium sulfate, filtered, and concentrated under reduced pressure. The crude solid was purified by silica gel column chromatography (1% to 50% EtOAc/PET) and the batches combined to provide tert-butyl N-(5-methoxypyridazin-3-yl)carbamate (10.64 g, 68% yield) as a white solid. LCMS (MM-ES+APCI, Pos): m/z 226.1 (M+H).

Step B. 5-methoxypyridazin-3-amine. To a solution of tert-butyl N-(5-methoxypyridazin-3-yl)carbamate (10.64 g, 47.2 mmol) in DCM (50 mL) was added TFA (53.2 mL). The reaction was stirred at 25° C. for 4 h. The mixture was concentrated in vacuo and combined with another batch (12.96 g, 57.5 mmol). The mixture was slowly diluted with water (200 mL) and neutralized (pH=7 to 8) with sat. aq. NaHCO₃. The aqueous solution was extracted with 25% IPA/DCM (150 mL×20) and the combined organic layers were washed with brine (1000 mL), dried over anhydrous sodium sulfate, filtered, and concentrated in vacuo to afford 5-methoxypyridazin-3-amine (9.34 g, 71% yield) as a yellow solid. LCMS (MM-ES+APCI, Pos): m/z 126.1 (M+H).

Step C. 7-methoxyimidazo[1,2-b]pyridazine. To a solution of 5-methoxypyridazin-3-amine (3.79 g, 30.3 mmol) in IPA (70 mL) was added 2-chloroacetaldehyde (13.7 mL, 212 mmol). The reaction was heated to 80° C. and stirred for 20 h. A second batch of 5-methoxypyridazin-3-amine (5.55 g, 44.4 mmol) and 2-chloroacetaldehyde (20 mL, 310 mmol) in IPA (80 mL) was stirred at 80° C. and stirred for 16 h. The two batches were combined and the mixture was concentrated under reduced pressure. The crude residue was purified via trituration in EtOAc (20 mL) at 25° C. for 1 h. Filtration yielded the solid product and drying under vacuum afforded 7-methoxyimidazo[1,2-b]pyridazine 14 (12.0 g, quant. yield) as a brown solid. LCMS (MM-ES+APCI, Pos): m/z 150.1 (M+H). Step D. imidazo[1,2-b]pyridazin-7-ol. Two batches in parallel containing 7-methoxyimidazo[1,2-b]pyridazine (6.0 g, 40.2 mmol) and DCE (120 mL) were cooled to 0° C. BBr₃ (27.1 mL, 282 mmol) was added dropwise and the mixture was heated to 80° C. for 6 h. The reaction was quenched slowly with ice water (50 mL) and concentrated in vacuo. The residue was diluted with 30% IPA/DCM (120 mL) and stirred for 30 min. The suspension was filtered and rinsed with 30% IPA/DCM (20 mL). The filtrate was concentrated under reduced pressure and the crude residue was purified via trituration in 30% IPA/DCM (80 mL). Filtration provided solid product. The filtrate was concentrated, water was added, and the mixture lyophilized to provide additional solid product. The two batches were combined to afford imidazo[1,2-b]pyridazin-7-ol (10.0 g, 92% yield) as a brown solid. LCMS (MM-ES+APCI, Pos): m/z 136.1 (M+H).

### Example 19 (I82)

### 4-bromo-2-fluoro-3-(methyl-d3)aniline

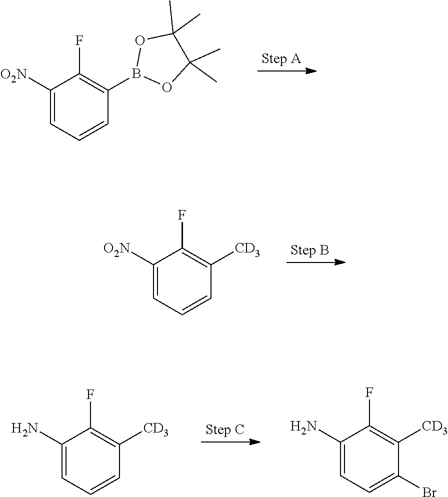 [SMILES: CC1(C)OB(c2cccc([N+](=O)[O-])c2F)OC1(C)C ; [2H]C([2H])([2H])c1cccc([N+](=O)[O-])c1F ; [2H]C([2H])([2H])c1cccc(N)c1F ; [2H]C([2H])([2H])c1c(Br)ccc(N)c1F]

Step A. 2-fluoro-1-(methyl-d3)-3-nitrobenzene. A solution of 2-(2-fluoro-3-nitrophenyl)-4,4,5,5-tetramethyl-1,3,2-dioxaborolane (14.5 g, 54.3 mmol), CsF (28.9 g, 190 mmol), Pd(Amphos)Cl₂ (1.92 g, 2.71 mmol) in DMF (167 mL) and water (102 mL) was purged with N₂. CD₃I (6.8 mL, 109 mmol) was added, and the reaction was heated to 45° C. for 2 h. The mixture was diluted with water (45 mL) and extracted with EtOAc (2×75 mL). The combined organic layers were dried over MgSO₄, filtered, and concentrated to dryness. The crude material was purified via silica gel column chromatography (10% EtOAc/Heptanes) to afford 2-fluoro-1-(methyl-d3)-3-nitrobenzene (6.77 g, 78% yield) as a yellow solid.

Step B. 2-fluoro-3-(methyl-d3)aniline. To a solution of 2-fluoro-1-(methyl-d3)-3-nitrobenzene (6.80 g, 43.0 mmol) in EtOH (102 mL) and water (34 mL) was added Fe powder (7.20 g, 129 mmol) and NH₄Cl (6.90 g, 129 mmol). The mixture was stirred at 80° C. for 2 h and cooled to 30° C. The suspension was filtered and rinsed with EtOH (35 mL). The filtrate was concentrated under reduced pressure, diluted with water (35 mL), and extracted with EtOAc (2×70 mL). The combined organics were washed with brine, dried over MgSO₄, filtered, and concentrated to afford 2-fluoro-3-(methyl-d3)aniline (7.0 g, 88% yield) as a pale yellow oil.

Step C. 4-bromo-2-fluoro-3-(methyl-d3)aniline. A solution of 2-fluoro-3-(methyl-d3)aniline (4.56 g, 35.6 mmol) in MeCN (23 mL) was placed under a N₂ atmosphere and cooled to −20° C. A solution of NBS (6.33 g, 35.6 mmol) in MeCN (46 mL) was added dropwise. The mixture was stirred at −20° C. for 5 min, quenched with sat. NaHSO₃ (23 mL), and extracted with MTBE (9 mL). The organic phase was concentrated to dryness, and the residue was redissolved in MTBE (46 mL). The organic mixture was washed with 10% NaOH (23 mL), brine (23 mL), dried over MgSO₄, and filtered. After concentration, the residue was purified via slurry with heptanes (14 mL) at −20° C. for 2 h. Filtration and drying in vacuo afforded 4-bromo-2-fluoro-3-(methyl-d3)aniline (3.9 g, 53% yield) as a pink solid. LCMS (MM-ES+APCI, Pos): m/z 207.0, 209.0 (M+H).

### Example 20 (I6)

### 2,3-dimethyl-4-((1-methyl-1H-benzo[d][1,2,3]triazol-5-yl)oxy)aniline

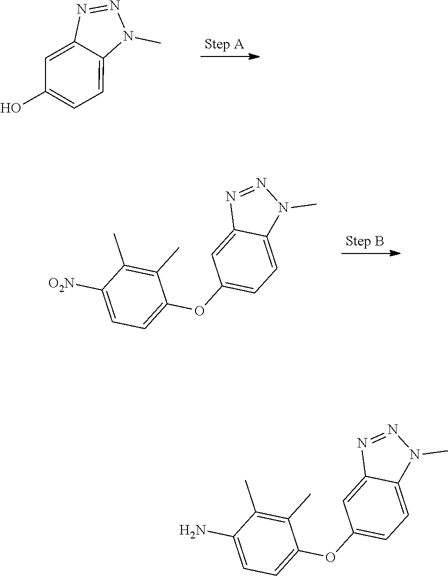 [SMILES: Cn1nnc2cc(O)ccc21 ; Cc1c(Oc2ccc3c(c2)nnn3C)ccc([N+](=O)[O-])c1C ; Cc1c(N)ccc(Oc2ccc3c(c2)nnn3C)c1C]

Step A. 5-(2,3-dimethyl-4-nitrophenoxy)-1-methyl-H-benzo[d][1,2,3]triazole. To a solution of 1-fluoro-2,3-dimethyl-4-nitrobenzene (500 mg, 2.96 mmol) in DMF (10 mL) was added Cs₂CO₃ (1.93 g, 5.91 mmol) and 1-methylbenzotriazol-5-ol (371 mg, 2.36 mmol). The mixture was stirred at 50° C. for 2 h, diluted with water (10 mL), and extracted with EtOAc (20 mL×3). The combined organic layers were washed with brine (20 mL), dried over anhydrous Na₂SO₄, filtered and concentrated under reduced pressure. The crude residue was purified by silica gel column chromatography (5% to 25% EtOAc/PET) to provide 5-(2,3-dimethyl-4-nitro-phenoxy)-1-methyl-benzotriazole (800 mg, quant.) as a brown oil. LCMS (MM-ES+APCI, Pos): m/z 299.1 (M+H).

Step B. 2,3-dimethyl-4-((1-methyl-1H-benzo[d][1,2,3]triazol-5-yl)oxy)aniline. To a solution of 5-(2,3-dimethyl-4-nitro-phenoxy)-1-methyl-benzotriazole (800 mg, 2.47 mmol) in EtOH (10 mL) and water (5 mL) was added Fe powder (551 mg, 9.87 mmol) and NH₄Cl (528 mg, 9.87 mmol). The mixture was stirred at 85° C. for 1 h and cooled to ambient temperature. The suspension was filtered and rinsed with EtOH (10 mL). The filtrate was concentrated under reduced pressure to afford 2,3-dimethyl-4-(1-methylbenzotriazol-5-yl)oxy-aniline (500 mg, 69% yield) as a yellow solid. LCMS (MM-ES+APCI, Pos): m/z 269.0 (M+H). Intermediates I7-I23, I83-I102 in Table 2 below were prepared according to Example 20 Steps A to B. Step A can be carried out using Cs₂CO₃ or K₂CO₃ as base, and DMF or MeCN as solvent.

TABLE 2
A list of intermediate compounds I7-I23, I83-I102

| | | | MS |
|---|---|---|---|
| | | | API |
| Int. ID | Structure | Chemical Name | (m/z) |
| I7 | 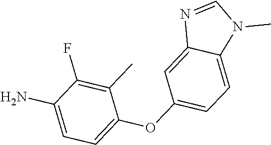 [SMILES: Cc1c(Oc2ccc3c(c2)ncn3C)ccc(N)c1F] | 2-fluoro-3-methyl-4-((1-meth- yl-1H-benzo[d]imidazol-5-yl)- oxy)aniline | 272.0 (M + H) |
| I8 | 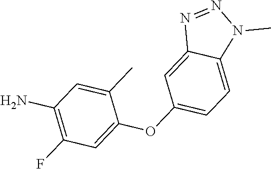 [SMILES: Cc1cc(N)c(F)cc1Oc1ccc2c(c1)nnn2C] | 2-fluoro-5-methyl-4-((1-meth- yl-1H-benzo[d][1,2,3]triazol- 5-yl)oxy)aniline | 273.1 (M + H) |
| I9 | 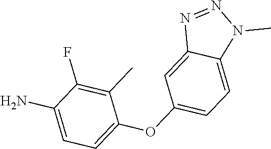 [SMILES: Cc1c(Oc2ccc3c(c2)nnn3C)ccc(N)c1F] | 2-fluoro-3-methyl-4-((1-meth- yl-1H-benzo[d][1,2,3]triazol- 5-yl)oxy)aniline | 273.0 (M + H) |
| I10 | 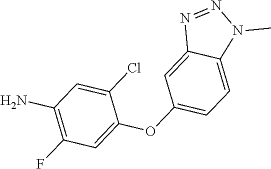 [SMILES: Cn1nnc2cc(Oc3cc(F)c(N)cc3Cl)ccc21] | 5-chloro-2-fluoro-4-((1-meth- yl-1H-benzo[d][1,2,3]triazol- 5-yl)oxy)aniline | 293.0 (M + H) |
| I11 | 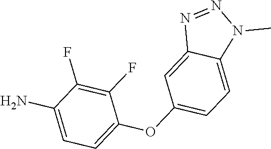 [SMILES: Cn1nnc2cc(Oc3ccc(N)c(F)c3F)ccc21] | 2,3-difluoro-4-((1-methyl-1H- benzo[d][1,2,3]triazol-5-yl)- oxy)aniline | 277.1 (M + H) |
| I12 | 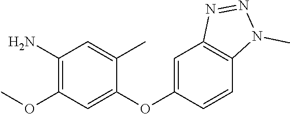 | 2-methoxy-5-methyl-4-((1- methyl-1H-benzo[d][1,2,3] triazol-5-yl)oxy)aniline | 285.1 (M + H) |
| I13 | 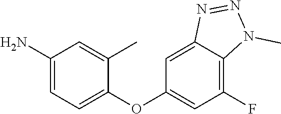 [SMILES: Cc1cc(N)ccc1Oc1cc(F)c2c(c1)nnn2C] | 4-((7-fluoro-1-methyl-1H- benzo[d][1,2,3]triazol-5-yl)- oxy)-3-methylaniline | 273.1 (M + H) |
| I14 | 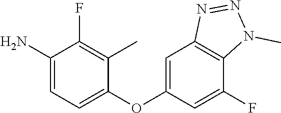 [SMILES: Cc1c(Oc2cc(F)c3c(c2)nnn3C)ccc(N)c1F] | 2-fluoro-4-((7-fluoro-1-meth- yl-1H-benzo[d][1,2,3]triazol- 5-yl)oxy)-3-methylaniline | 291.3 (M + H) |
| I15 | 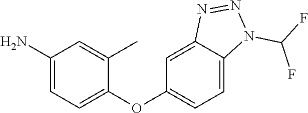 [SMILES: Cc1cc(N)ccc1Oc1ccc2c(c1)nnn2C(F)F] | 4-((1-(difluoromethyl)-1H- benzo[d][1,2,3]triazol-5-yl)- oxy)-3-methylaniline | 291.1 (M + H) |
| I16 | 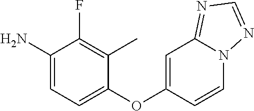 [SMILES: Cc1c(Oc2ccn3ncnc3c2)ccc(N)c1F] | 4-([1,2,4]triazolo[1,5-a]pyri- din-7-yloxy)-2-fluoro-3-meth- ylaniline | 259.4 (M + H) |
| I17 | 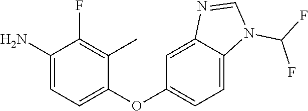 [SMILES: Cc1c(Oc2ccc3c(c2)ncn3C(F)F)ccc(N)c1F] | 4-((1-(difluoromethyl)-1H- benzo[d]imidazol-5-yl)oxy)- 2-fluoro-3-methylaniline | 307.9 (M + H) |
| I18 | 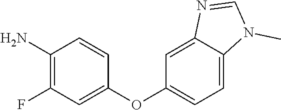 [SMILES: Cn1cnc2cc(Oc3ccc(N)c(F)c3)ccc21] | 2-fluoro-4-((1-methyl-1H- benzo[d][1,2,3]triazol-5-yl)- oxy)aniline | 259.1 (M + H) |
| I19 | 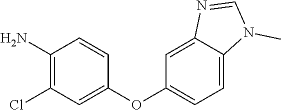 [SMILES: Cn1cnc2cc(Oc3ccc(N)c(Cl)c3)ccc21] | 2-chloro-4-((1-methyl-1H- benzo[d][1,2,3]triazol-5-yl)- oxy)aniline | 275.3 (M + H) |
| I20 | 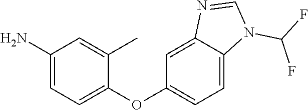 [SMILES: Cc1cc(N)ccc1Oc1ccc2c(c1)ncn2C(F)F] | 4-((1-(difluoromethyl)-1H- benzo[d]imidazol-5-yl)oxy)- 3-methylaniline | 290.1 (M + H) |
| I21 | 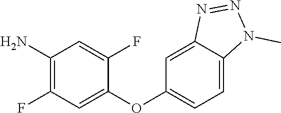 [SMILES: Cn1nnc2cc(Oc3cc(F)c(N)cc3F)ccc21] | 2,5-difluoro-4-((1-methyl-1H- benzo[d][1,2,3]triazol-5-yl)- oxy)aniline | 277.2 (M + H) |
| I22 | 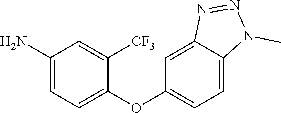 [SMILES: Cn1nnc2cc(Oc3ccc(N)cc3C(F)(F)F)ccc21] | 4-((1-methyl-1H-benzo[d] [1,2,3]triazol-5-yl)oxy)-3- (trifluoromethyl)aniline | 309.1 (M + H) |
| I23 | 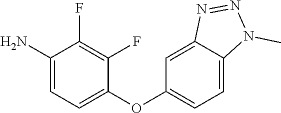 [SMILES: Cn1nnc2cc(Oc3ccc(N)c(F)c3F)ccc21] | 2,3-difluoro-4-((1-methyl-1H- benzo[d][1,2,3]triazol-5-yl)- oxy)aniline | 277.2 (M + H) |
| I83 | 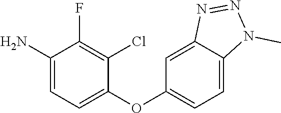 [SMILES: Cn1nnc2cc(Oc3ccc(N)c(F)c3Cl)ccc21] | 3-chloro-2-fluoro-4-((1-meth- yl-1H-benzo[d][1,2,3]triazol- 5-yl)oxy)aniline | 293.0 (M + H) |
| I84 | 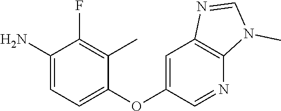 [SMILES: Cc1c(Oc2cnc3c(c2)ncn3C)ccc(N)c1F] | 2-fluoro-3-methyl-4-((3-meth- yl-3H-imidazo[4,5-b]pyridin- 6-yl)oxy)aniline | 273.2 (M + H) |
| I85 | 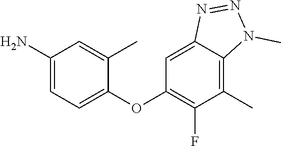 [SMILES: Cc1cc(N)ccc1Oc1cc2nnn(C)c2c(C)c1F] | 4-((6-fluoro-1,7-dimethyl-1H- benzo[d][1,2,3]triazol-5-yl)- oxy)-3-methylaniline | 287.0 (M + H) |
| I86 | 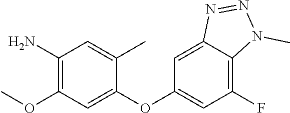 | 4-((7-fluoro-1-methyl-1H- benzo[d][1,2,3]triazol-5-yl)- oxy)-2-methoxy-5-methyl- aniline | 303.1 (M + H) |
| I87 | 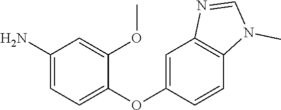 [SMILES: COc1cc(N)ccc1Oc1ccc2c(c1)ncn2C] | 3-methoxy-4-((1-methyl-1H- benzo[d]imidazol-5-yl)oxy)- aniline | 270.0 (M + H) |
| I88 | 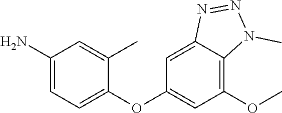 [SMILES: COc1cc(Oc2ccc(N)cc2C)cc2nnn(C)c12] | 4-((7-methoxy-1-methyl-1H- benzo[d][1,2,3]triazol-5-yl)- oxy)-3-methylaniline | 285.2 (M + H) |
| I89 | 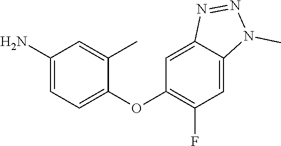 [SMILES: Cc1cc(N)ccc1Oc1cc2nnn(C)c2cc1F] | 4-((6-fluoro-1-methyl-1H- benzo[d][1,2,3]triazol-5-yl)- oxy)-3-methylaniline | 273.2 (M + H) |
| I90 | 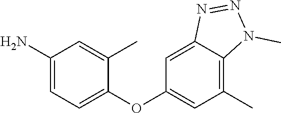 [SMILES: Cc1cc(N)ccc1Oc1cc(C)c2c(c1)nnn2C] | 4-((1,7-dimethyl-1H-benzo [d][1,2,3]triazol-5-yl)oxy)-3- methylaniline | 269.2 (M + H) |
| I91 | 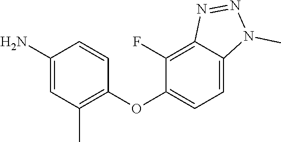 [SMILES: Cc1cc(N)ccc1Oc1ccc2c(nnn2C)c1F] | 4-((4-fluoro-1-methyl-1H- benzo[d][1,2,3]triazol-5-yl)- oxy)-3-methylaniline | 273.0 (M + H) |
| I92 | 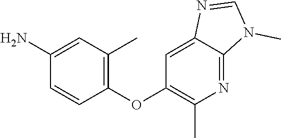 [SMILES: Cc1cc(N)ccc1Oc1cc2ncn(C)c2nc1C] | 4-((3,5-dimethyl-3H-imidazo [4,5-b]pyridin-6-yl)oxy)-3- methylaniline | 269.0 (M + H) |
| I93 | 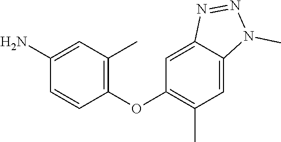 [SMILES: Cc1cc(N)ccc1Oc1cc2nnn(C)c2cc1C] | 4-((1,6-dimethyl-1H-benzo [d][1,2,3]triazol-5-yl)oxy)-3- methylaniline | 269.3 (M + H) |
| I94 | 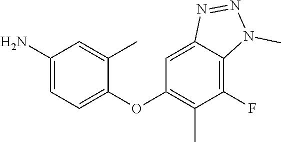 [SMILES: Cc1cc(N)ccc1Oc1cc2nnn(C)c2c(F)c1C] | 4-((7-fluoro-1,6-dimethyl-1H- benzo[d][1,2,3]triazol-5-yl)- oxy)-3-methylaniline | 287.3 (M + H) |
| I95 | 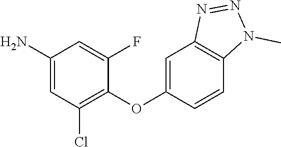 [SMILES: Cn1nnc2cc(Oc3c(F)cc(N)cc3Cl)ccc21] | 3-chloro-5-fluoro-4-((1-meth- yl-1H-benzo[d][1,2,3]triazol- 5-yl)oxy)aniline | 293.1 (M + H) |
| I96 | 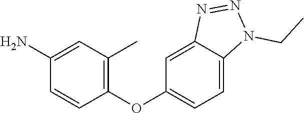 [SMILES: CCn1nnc2cc(Oc3ccc(N)cc3C)ccc21] | 4-((1-ethyl-1H-benzo[d][1,2,3] triazol-5-yl)oxy)-3-methyl- aniline | 269.2 (M + H) |
| I97 | 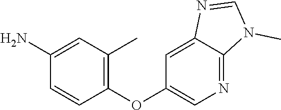 [SMILES: Cc1cc(N)ccc1Oc1cnc2c(c1)ncn2C] | 3-methyl-4-((3-methyl-3H- imidazo[4,5-b]pyridin-6-yl)- oxy)aniline | 255.2 (M + H) |
| I98 | 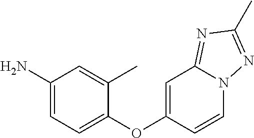 [SMILES: Cc1nc2cc(Oc3ccc(N)cc3C)ccn2n1] | 3-methyl-4-((2-methyl-[1,2,4] triazolo[1,5-a]pyridin-7-yl)- oxy)aniline | 255.3 (M + H) |
| I99 | 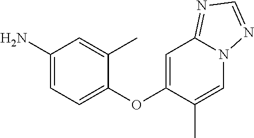 [SMILES: Cc1cc(N)ccc1Oc1cc2ncnn2cc1C] | 3-methyl-4-((6-methyl-[1,2,4] triazolo[1,5-a]pyridin-7-yl)- oxy)aniline | 255.3 (M + H) |
| I100 | 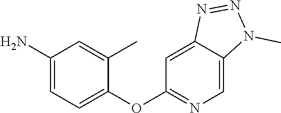 [SMILES: Cc1cc(N)ccc1Oc1cc2nnn(C)c2cn1] | 3-methyl-4-((3-methyl-3H- [1,2,3]triazolo[4,5-c]pyridin- 6-yl)oxy)aniline | 256.0 (M + H) |
| I101 | 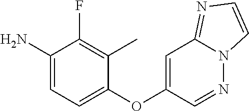 [SMILES: Cc1c(Oc2cnn3ccnc3c2)ccc(N)c1F] | 2-fluoro-4-(imidazo[1,2-b] pyridazin-7-yloxy)-3-methyl- aniline | 259.3 (M + H) |
| I102 | 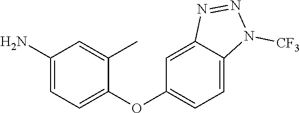 [SMILES: Cc1cc(N)ccc1Oc1ccc2c(c1)nnn2C(F)(F)F] | 3-methyl-4-((1-(trifluorometh- yl)-1H-benzo[d][1,2,3]triazol- 5-yl)oxy)aniline | 309.0 (M + H) |

### Example 21 (124)

### 3-methyl-4-((1-methyl-1H-benzo[1d][1,2,3]triazol-5-yl)oxy)aniline

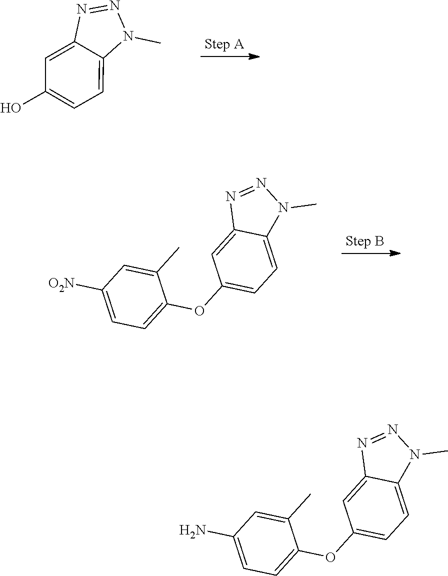 [SMILES: Cn1nnc2cc(O)ccc21 ; Cc1cc([N+](=O)[O-])ccc1Oc1ccc2c(c1)nnn2C ; Cc1cc(N)ccc1Oc1ccc2c(c1)nnn2C]

Step A. 1-methyl-5-(2-methyl-4-nitrophenoxy)-1H-benzo[d][1,2,3]triazole. 1-methyl-1H-benzo[d][1,2,3]triazol-5-ol (14.6 g, 98.3 mmol), 1-fluoro-2-methyl-4-nitro-benzene (15.3 g, 98.3 mmol) and K₂CO₃ (27.2 g, 196.6 mmol) were taken up in DMF (164 mL, 98.3 mmol) and heated to 90° C. for 40 min under N₂. The reaction was cooled to ambient temperature, diluted with 250 mL water and ice, stirred for 30 min, and filtered. The beige solid was rinsed with water and dried in vacuo to give 1-methyl-5-(2-methyl-4-nitrophenoxy)-1H-benzo[d][1,2,3]triazole (27.9 g, 98% yield). LCMS (MM-ES+APCI, Pos): m/z 285.1 (M+H).

Step B. 3-methyl-4-((1-methyl-1H-benzo[d][1,2,3]triazol-5-yl)oxy)aniline. A flask containing 1-methyl-5-(2-methyl-4-nitrophenoxy)-1H-benzo[d][1,2,3]triazole (1.00 g, 3.52 mmol) and Pd/C (749 mg, 0.35 mmol, 5% wt.) was sealed and purged with N₂. MeOH (13 mL) and ammonium formate (1.11 g, 17.6 mmol) were added, and the reaction was stirred for 1 h. The mixture was filtered over diatomite, eluted with MeOH, and the filtrate was concentrated in vacuo to afford 3-methyl-4-((1-methyl-1H-benzo[d][1,2,3]triazol-5-yl)oxy)aniline (833 mg, 93% yield) as an off-white powder. LCMS (MM-ES+APCI, Pos): m/z 255.2 (M+H).

### Example 22 (I25)

### N-(2-methoxy-5-methyl-4-((1-methyl-1H-benzo[d]imidazol-5-yl)oxy)phenyl)-6-(methylsulfinyl)pyrimido[5,4-d]pyrimidin-4-amine

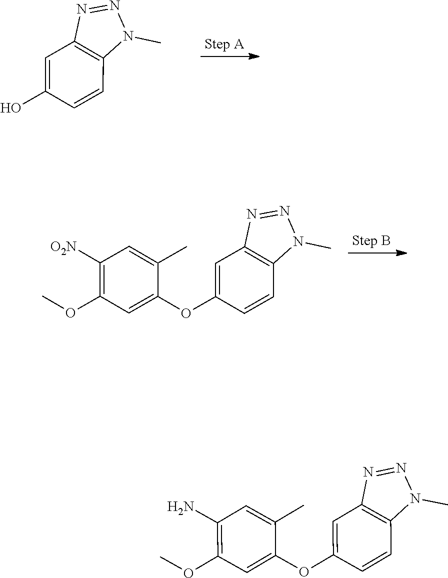

Step A. 6-(5-methoxy-2-methyl-4-nitrophenoxy)-1-methyl-1H-benzo[d]imidazole. A solution of 1-fluoro-5-methoxy-2-methyl-4-nitrobenzene (0.969 g, 5.23 mmol), 1-methyl-1H-benzo[d]imidazol-6-ol (775 mg, 5.23 mmol), Cs₂CO₃ (2.05 g, 6.28 mmol), and IPA (10.0 mL) was heated to 85° C. for 3 h. The mixture was filtered and concentrated in vacuo. The crude material was purified by silica gel chromatography (0% to 100% EtOAc/Hexanes) to afford 6-(5-methoxy-2-methyl-4-nitrophenoxy)-1-methyl-1H-benzo[d]imidazole (1.10 g, 66% yield). LCMS (MM-ES+APCI, Pos): m/z 314.1 (M+H).

Step B. 2-methoxy-5-methyl-4-((1-methyl-1H-benzo[d]imidazol-5-yl)oxy)aniline. 5-(5-methoxy-2-methyl-4-nitrophenoxy)-1-methyl-1H-benzo[d]imidazole (950 mg, 3.03 mmol) and 5% Pd/Al₂O₃ (645 mg, 303 μmol) were suspended in MeOH (15.2 mL) and sparged with H₂ gas for 10 min. The reaction was sealed, stirred under H₂ (1 atm) at 25° C. for 16 h, and filtered to afford 2-methoxy-5-methyl-4-((1-methyl-1H-benzo[d]imidazol-5-yl)oxy)aniline (453 mg, 53% yield). LCMS (MM-ES+APCI, Pos): m/z 284.1 (M+H).

### Example 23 (I26)

### 2,3,6-trifluoro-4-((1-methyl-1H-benzo[d][1,2,3]triazol-5-yl)oxy)aniline

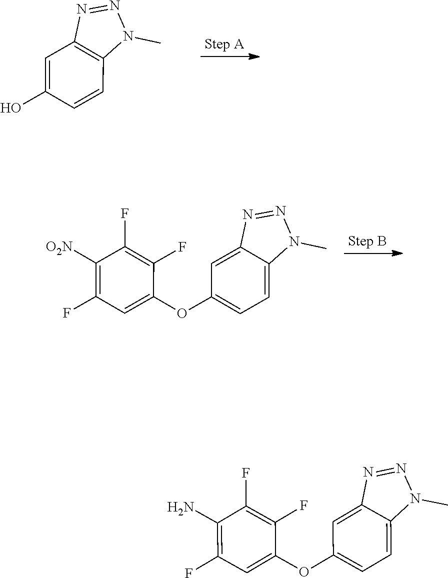 [SMILES: Cn1nnc2cc(O)ccc21 ; Cn1nnc2cc(Oc3cc(F)c([N+](=O)[O-])c(F)c3F)ccc21 ; Cn1nnc2cc(Oc3cc(F)c(N)c(F)c3F)ccc21]

Step A. 1-methyl-5-(2,3,5-trifluoro-4-nitrophenoxy)-1H-benzo[d][1,2,3]triazole. To a solution of 1,2,3,5-tetrafluoro-4-nitrobenzene (500 mg, 2.56 mmol), 1-methyl-1H-benzo[d][1,2,3]triazol-5-ol (382 mg, 2.56 mmol), and tetrabutylammonium hydrogen sulfate (87 mg, 0.26 mmol) in DCM (12.8 mL) was added NaOH (2M aq., 1.3 mL). The reaction was stirred at 23° C. for 16 h. The solution was washed with brine, dried over Na₂SO₄, filtered, and concentrated in vacuo. The resulting residue was purified via silica gel chromatography to obtain 1-methyl-5-(2,3,5-trifluoro-4-nitrophenoxy)-1H-benzo[d][1,2,3]triazole (143 mg, 17%). LCMS (MM-ES+APCI, Pos): m/z 325.0 (M+H).

Step B. 2,3,6-trifluoro-4-((1-methyl-1H-benzo[d][1,2,3]triazol-5-yl)oxy)aniline. A slurry of 1-methyl-5-(2,3,5-trifluoro-4-nitrophenoxy)-1H-benzo[d][1,2,3]triazole (143 mg, 0.44 mmol) and Pd/C (141 mg, 0.13 mmol, 10% wt.) in MeOH (4.4 mL) was sparged with H₂ for 10 min. The reaction was sealed and stirred at 23° C. under H₂ (1 atm) for 17.5 h. The reaction was filtered over celite, and the pad was washed with MeOH (15 mL). The filtrate was concentrated in vacuo to obtain 2,3,6-trifluoro-4-((1-methyl-1H-benzo[d][1,2,3]triazol-5-yl)oxy)aniline (125 mg, 96%) as an off-white solid. LCMS (MM-ES+APCI, Pos): m/z 295.1 (M+H).

### Example 24 (I27)

### 2,6-difluoro-4-((1-methyl-1H-benzo[d][1,2,3]triazol-5-yl)oxy)aniline

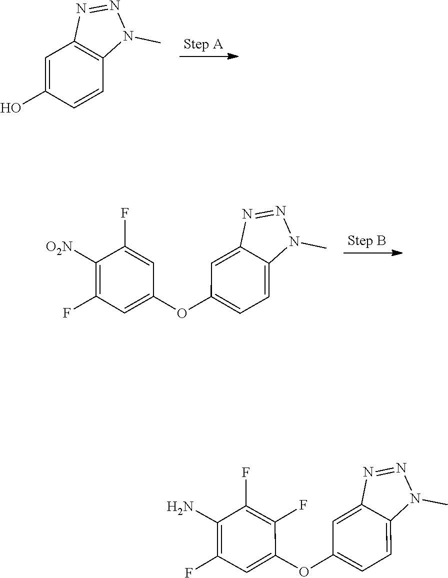 [SMILES: Cn1nnc2cc(O)ccc21 ; Cn1nnc2cc(Oc3cc(F)c([N+](=O)[O-])c(F)c3)ccc21 ; Cn1nnc2cc(Oc3cc(F)c(N)c(F)c3F)ccc21]

2,6-difluoro-4-((1-methyl-1H-benzo[d][1,2,3]triazol-5-yl)oxy)aniline. Prepared according to Example 23 Steps A to B, substituting 1,3,5-trifluoro-2-nitrobenzene for 1,2,3,5-tetrafluoro-4-nitrobenzene to afford 2,6-difluoro-4-((1-methyl-1H-benzo[d][1,2,3]triazol-5-yl)oxy)aniline (56 mg, 11%). LCMS (MM-ES+APCI, Pos): m/z 277.1 (M+H).

### Example 25 (I28)

### 2-fluoro-3-methoxy-4-((1-methyl-1H-benzo[d][1,2,3]triazol-5-yl)oxy)aniline

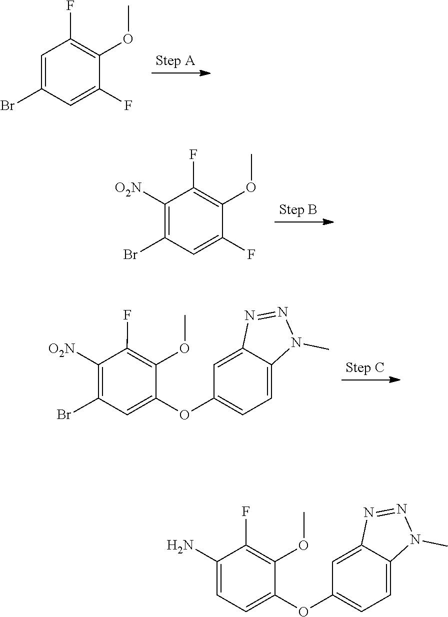 [SMILES: COc1c(F)cc(Br)cc1F ; COc1c(F)cc(Br)c([N+](=O)[O-])c1F ; COc1c(Oc2ccc3c(c2)nnn3C)cc(Br)c([N+](=O)[O-])c1F ; COc1c(Oc2ccc3c(c2)nnn3C)ccc(N)c1F]

Step A. 1-bromo-3,5-difluoro-4-methoxy-2-nitrobenzene. A solution of 5-bromo-1,3-difluoro-2-methoxy-benzene (4.0 g, 17.9 mmol) in H₂SO₄ (30 mL) was cooled to 0° C., and KNO₃ (2.18 g, 21.5 mmol) was added portion wise. The mixture was stirred for 1 h at 25° C. and slowly poured into H₂O (100 mL) at 0° C. The aqueous solution was neutralized (pH 7) using NaHCO₃ and the product was extracted with EtOAc (100 mL×3). The combined organic layers were washed with brine (150 mL), dried over anhydrous Na₂SO₄, filtered, and concentrated under reduced pressure to afford 1-bromo-3,5-difluoro-4-methoxy-2-nitro-benzene (4.2 g, 88% yield) as a yellow oil.

Step B. 5-(5-bromo-3-fluoro-2-methoxy-4-nitrophenoxy)-1-methyl-1H-benzo[d][1,2,3]triazole. To a solution of 1-bromo-3,5-difluoro-4-methoxy-2-nitro-benzene (2.00 g, 7.46 mmol) and 1-methylbenzotriazol-5-ol (0.67 g, 4.48 mmol) in DMF (20 mL) was added K₂CO₃ (2.06 g, 14.9 mmol). The mixture was stirred at 80° C. for 1 h, diluted with water (50 mL), and extracted with EtOAc (50 mL×3). The combined organic layers were washed with brine (50 mL×3), dried over anhydrous Na₂SO₄, filtered, and concentrated under reduced pressure. The crude oil was purified by preparative HPLC to afford 5-(5-bromo-3-fluoro-2-methoxy-4-nitro-phenoxy)-1-methyl-benzotriazole (450 mg, 1.13 mmol, 25% yield) as a yellow solid. LCMS (MM-ES+APCI, Pos): m/z 397.0, 398.9 (M+H).

Step C. 2-fluoro-3-methoxy-4-((1-methyl-1H-benzo[d][1,2,3]triazol-5-yl)oxy)aniline. To a solution of 5-(5-bromo-3-fluoro-2-methoxy-4-nitro-phenoxy)-1-methyl-benzotriazole (200 mg, 0.50 mmol) in THF (50 mL) was added 10% Pd/C (200 mg, 25 μmol). The flask was evacuated and backfilled with H₂ thrice. The mixture was stirred for 2 h at 25° C. under an atmosphere of H₂ (3 atm), and the suspension was filtered through a pad of Celite eluting with MeOH (50 mL). The crude material was purified by preparative TLC (10% MeOH/DCM) to afford 2-fluoro-3-methoxy-4-(1-methylbenzotriazol-5-yl)oxy-aniline (50 mg, 34% yield) as a yellow solid. LCMS (MM-ES+APCI, Pos): m/z 289.0 (M+H)

### Example 26 (I103)

### 3-chloro-5-methyl-4-((1-methyl-1H-benzo[d][1,2,3]triazol-5-yl)oxy)aniline

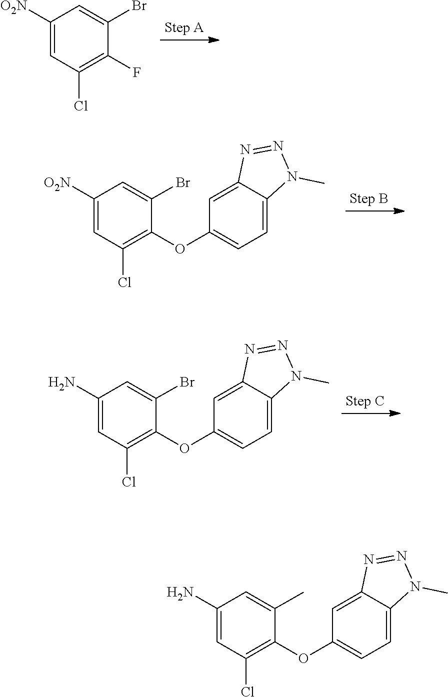 [SMILES: O=[N+]([O-])c1cc(Cl)c(F)c(Br)c1 ; Cn1nnc2cc(Oc3c(Cl)cc([N+](=O)[O-])cc3Br)ccc21 ; Cn1nnc2cc(Oc3c(Cl)cc(N)cc3Br)ccc21 ; Cc1cc(N)cc(Cl)c1Oc1ccc2c(c1)nnn2C]

Step A. 5-(2-bromo-6-chloro-4-nitrophenoxy)-1-methyl-H-benzo[d][1,2,3]triazole. To a solution of 1-bromo-3-chloro-2-fluoro-5-nitro-benzene (200 mg, 0.79 mmol) and 1-methylbenzotriazol-5-ol (117 mg, 0.79 mmol) in DMF (2.5 mL) was added Cs₂CO₃ (512 mg, 1.57 mmol). The reaction was stirred at 25° C. for 1.5 h. The mixture was diluted with water, the layers were separated, and the aqueous phase was extracted with EtOAc (8 mL×3). The combined organic layers were washed with brine (6 mL×3), dried over anhydrous sodium sulfate, and filtered. The filtrate was concentrated under reduced pressure to give 5-(2-bromo-6-chloro-4-nitro-phenoxy)-1-methyl-benzotriazole (290 mg, 94% yield) as a brown solid. LCMS (MM-ES+APCI, Pos): m/z 383.0, 384.9 (M+H).

Step B. 3-bromo-5-chloro-4-((1-methyl-1H-benzo[d][1,2,3]triazol-5-yl)oxy)aniline. To a solution of 5-(2-bromo-6-chloro-4-nitro-phenoxy)-1-methyl-benzotriazole (290 mg, 0.74 mmol) in EtOH (6 mL) and water (2 mL) was added NH₄Cl (238 mg, 4.45 mmol). The mixture was heated to 60° C. and Fe (207 mg, 3.70 mmol) was added. The reaction mixture was stirred at 80° C. for 1 h, diluted with MeOH (20 mL), and filtered through celite. The filtrate was concentrated under reduced pressure. The crude product was diluted with water (20 mL) and extracted with EtOAc (12 mL×3). The organic layers were dried over anhydrous sodium sulfate, filtered, and concentrated under reduced pressure to give 3-bromo-5-chloro-4-(1-methylbenzotriazol-5-yl)oxy-aniline 4 (255 mg, 93% yield) as a brown oil. LCMS (MM-ES+APCI, Pos): m/z 353.0, 355.0 (M+H).

Step C. 3-chloro-5-methyl-4-((1-methyl-1H-benzo[d][1,2,3]triazol-5-yl)oxy)aniline. To a solution of 3-bromo-5-chloro-4-(1-methylbenzotriazol-5-yl)oxy-aniline (255 mg, 0.69 mmol) and methylboronic acid (41 mg, 0.69 mmol) in 1,4-dioxane (3 mL) and water (0.3 mL) were added K₂CO₃ (191 mg, 1.38 mmol) and Pd(dppf)Cl₂ (51 mg, 69 μmol). The reaction was purged with N₂ and stirred at 100° C. for 3 h. The mixture was transferred to a separatory funnel, diluted with water (15 mL) and extracted with EtOAc (8 mL×3). The combined organic layers were washed with brine (6 mL×3), dried over anhydrous sodium sulfate, filtered, and concentrated in vacuo. The residue was purified by silica gel column chromatography (25% to 50% EtOAc/PET) to afford product 3-chloro-5-methyl-4-(1-methylbenzotriazol-5-yl)oxy-aniline (115 mg, 55% yield) as a brown solid. LCMS (MM-ES+APCI, Pos): m/z 289.2 (M+H).

### Example 27 (I104)

### 2-fluoro-6-methoxy-3-methyl-4-((1-methyl-H-benzo[d][1,2,3]triazol-5-yl)oxy)aniline

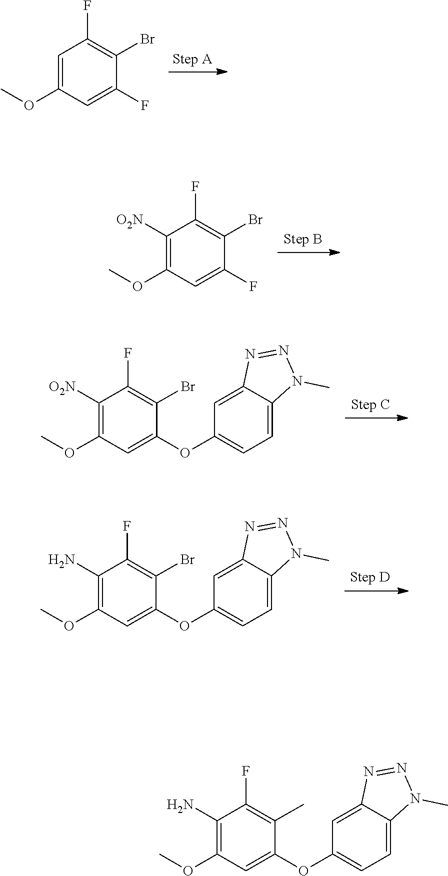

Step A. 2-bromo-1,3-difluoro-5-methoxy-4-nitrobenzene. To an ice cold solution of 2-bromo-1,3-difluoro-5-methoxy-benzene (8.00 g, 35.9 mmol) in DCM (80 mL) was added fuming HNO₃ (8.99 mL, 199 mmol) dropwise at 0° C. under N₂ atmosphere. The mixture was warmed to ambient temperature and stirred for 2 h. The reaction mixture was diluted with ice water (200 mL) and basified with sat. aq. NaHCO₃ (pH=8). The product was extracted with DCM (60 mL×3), and the combined organic layers were washed with brine (50 mL), dried over anhydrous sodium sulfate, filtered and concentrated in vacuo. The residue was purified by silica gel column chromatography (0% to 4% EtOAc/PET) to provide 2-bromo-1,3-difluoro-5-methoxy-4-nitro-benzene (9.0 g, 94% yield) as a white solid.

Steps B to D. 2-fluoro-6-methoxy-3-methyl-4-((1-methyl-1H-benzo[d][1,2,3]triazol-5-yl)oxy)aniline. Prepared according to Example 26 steps A to C, substituting 2-bromo-1,3-difluoro-5-methoxy-4-nitrobenzene for 1-bromo-3-chloro-2-fluoro-5-nitro-benzene to afford 2-fluoro-6-methoxy-3-methyl-4-((1-methyl-1H-benzo[d][1,2,3]triazol-5-yl)oxy)aniline (180 mg, 64% yield) as a brown solid. LCMS (MM-ES+APCI, Pos): m/z 302.8 (M+H).

### Example 28 (I105)

### 2-fluoro-4-((7-fluoro-1-methyl-1H-benzo[d]imidazol-5-yl)oxy)-6-methoxy-3-methylaniline

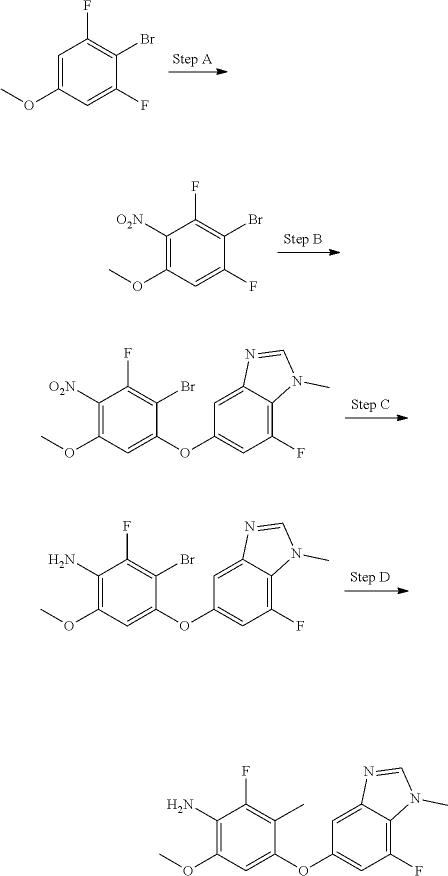

Steps A to D. 2-fluoro-4-((7-fluoro-1-methyl-1H-benzo[d]imidazol-5-yl)oxy)-6-methoxy-3-methylaniline. Prepared according to Example 27 steps A to D, substituting 7-fluoro-1-methyl-1H-benzo[d]imidazol-5-ol for 1-methylbenzotriazol-5-ol to afford 2-fluoro-4-((7-fluoro-1-methyl-1H-benzo[d]imidazol-5-yl)oxy)-6-methoxy-3-methylaniline (225 mg, 45% yield) as a yellow solid. LCMS (MM-ES+APCI, Pos): m/z 320.0 (M+H).

### Example 29 (I106)

### 3-fluoro-5-methyl-4-((1-methyl-1H-benzo[d][1,2,3]triazol-5-yl)oxy)aniline

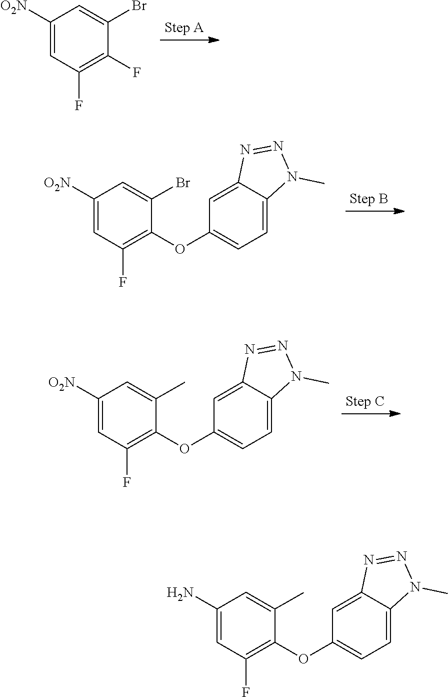 [SMILES: O=[N+]([O-])c1cc(F)c(F)c(Br)c1 ; Cn1nnc2cc(Oc3c(F)cc([N+](=O)[O-])cc3Br)ccc21 ; Cc1cc([N+](=O)[O-])cc(F)c1Oc1ccc2c(c1)nnn2C ; Cc1cc(N)cc(F)c1Oc1ccc2c(c1)nnn2C]

Step A. 5-(2-bromo-6-fluoro-4-nitrophenoxy)-1-methyl-1H-benzo[d][1,2,3]triazole. To a solution of 1-bromo-2,3-difluoro-5-nitro-benzene (200 mg, 0.84 mmol) and 1-methylbenzotriazol-5-ol (139 mg, 0.84 mmol) in DMF (8 mL) was added K₂CO₃ (290 mg, 2.10 mmol). The reaction was stirred at 80° C. for 1 h and diluted with water (100 mL). The mixture was transferred to a separatory funnel and the aqueous layer mixture was extracted with EtOAc (100 mL×2). The combined organic layers were washed with brine (100 mL×3), dried over anhydrous sodium sulfate, filtered, and concentrated under reduced pressure. The residue was purified by silica gel column chromatography (10% to 50% EtOAc/PET and 2% MeOH/DCM) to give 5-(2-bromo-6-fluoro-4-nitro-phenoxy)-1-methyl-benzotriazole (1.55 g, 70% yield) as a brown solid. LCMS (MM-ES+APCI, Pos): m/z 367.0, 368.9 (M+H).

Step B. To a solution of 5-(2-bromo-6-fluoro-4-nitro-phenoxy)-1-methyl-benzotriazole (300 mg, 0.80 mmol) and methylboronic acid (57 mg, 0.96 mmol) in 1,4-dioxane (5 mL) and H₂O (1 mL) were added Pd(dppf)C₁₂ (58 mg, 80 μmol) and K₂CO₃ (275 mg, 1.99 mmol). The reaction was purged with N₂, stirred at 90° C. for 2 h, and quenched with H₂O (100 mL). The biphasic mixture was separated, and the aqueous layer was extracted with EtOAc (100 mL×3). The combined organic layers were dried over anhydrous sodium sulfate, filtered, and concentrated under reduced pressure. The residue was purified by silica gel column chromatography (20% to 50% EtOAc/PET) to afford 5-(2-fluoro-6-methyl-4-nitro-phenoxy)-1-methyl-benzotriazole (300 mg, 23% yield) as a yellow solid. LCMS (MM-ES+APCI, Pos): m/z 303.0 (M+H).

Step C. To a solution of 5-(2-fluoro-6-methyl-4-nitro-phenoxy)-1-methyl-benzotriazole (230 mg, 0.71 mmol) in EtOH (2 mL) and H₂O (2 mL) were added Fe (157 mg, 2.81 mmol) and NH₄Cl (151 mg, 2.82 mmol). The mixture was stirred at 60° C. for 1 h, and the suspension was filtered eluting with EtOH (20 mL). The filtrate was concentrated in vacuo to afford 3-fluoro-5-methyl-4-(1-methylbenzotriazol-5-yl)oxy-aniline (360 mg, quant. yield) as an off-white solid. LCMS (MM-ES+APCI, Pos): m/z 273.1 (M+H).

### Example 30 (I107)

### 2,5-difluoro-3-methyl-4-((1-methyl-1H-benzo[d][1,2,3]triazol-5-yl)oxy)aniline

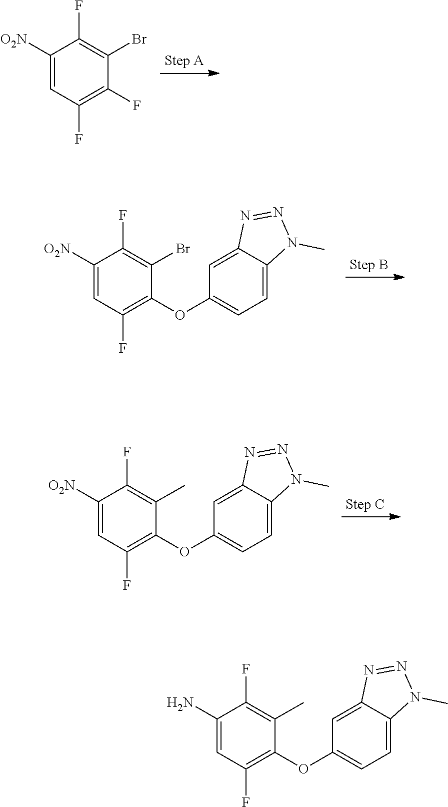 [SMILES: O=[N+]([O-])c1cc(F)c(F)c(Br)c1F ; Cn1nnc2cc(Oc3c(F)cc([N+](=O)[O-])c(F)c3Br)ccc21 ; Cc1c(F)c([N+](=O)[O-])cc(F)c1Oc1ccc2c(c1)nnn2C ; Cc1c(F)c(N)cc(F)c1Oc1ccc2c(c1)nnn2C]

Steps A to C. 2,5-difluoro-3-methyl-4-((1-methyl-1H-benzo[d][1,2,3]triazol-5-yl)oxy)aniline. Prepared according to Example 29 steps A to C, substituting 3-bromo-1,2,4-trifluoro-5-nitrobenzene for 1-bromo-2,3-difluoro-5-nitrobenzene to afford 2,5-difluoro-3-methyl-4-((1-methyl-1H-benzo[d][1,2,3]triazol-5-yl)oxy)aniline (1.60 g, 77% yield) as a pink solid. LCMS (MM-ES+APCI, Pos): m/z 291.1 (M+H).

### Example 31 (I108)

### 2-fluoro-4-((7-fluoro-1-methyl-1H-benzo[d]imidazol-5-yl)oxy)-3-methylaniline

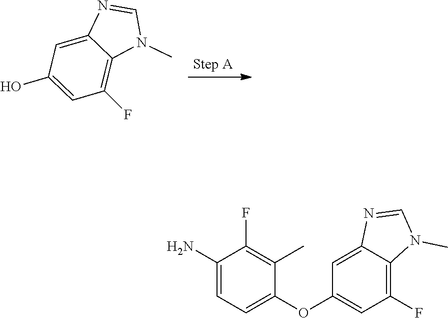 [SMILES: Cn1cnc2cc(O)cc(F)c21 ; Cc1c(Oc2cc(F)c3c(c2)ncn3C)ccc(N)c1F]

Step A. 2-fluoro-4-((7-fluoro-1-methyl-1H-benzo[d]imidazol-5-yl)oxy)-3-methylaniline. To a flask containing 7-fluoro-1-methyl-benzimidazol-5-ol (772 mg, 4.29 mmol), 2-fluoro-4-iodo-3-methyl-aniline (1 g, 3.57 mmol), Cui (204 mg, 1.07 mmol), picolinic acid (88 mg, 0.72 mmol) and K₃PO₄ (1.52 g, 7.14 mmol) was added DMSO (15 mL) at 25° C. The mixture was purged with N₂ and heated to 80° C. for 16 h. The reaction was quenched by with H₂O (50 mL), and the aqueous layer mixture was extracted with EtOAc (30 mL×3). The combined organic layers were washed with water (150 mL) and brine (80 mL), dried over anhydrous sodium sulfate, filtered, and concentrated under reduced pressure. The residue was purified by silica gel column chromatography (0% to 75% EtOAc/PET) to afford 2-fluoro-4-(7-fluoro-1-methyl-benzimidazol-5-yl) oxy-3-methyl-aniline (500 mg, 45% yield) as a pink solid. LCMS (MM-ES+APCI, Pos): m/z 290.2 (M+H).

Intermediates I108-I115 in Table 12 below were prepared according to Example 31 Step A.

TABLE 12
A list of intermediate compounds I108 to I115

| | | | MS |
|---|---|---|---|
| Int. | | | API |
| ID | Structure | Chemical Name | (m/z) |
| I108 | 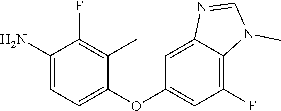 [SMILES: Cc1c(Oc2cc(F)c3c(c2)ncn3C)ccc(N)c1F] | 2-fluoro-4-((7-fluoro-1-methyl-1H- benzo[d]imidazol-5-yl)oxy)-3-meth- ylaniline | 290.2 (M + H) |
| I109 | 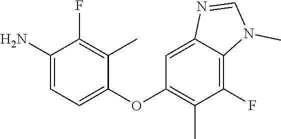 [SMILES: Cc1c(Oc2cc3ncn(C)c3c(F)c2C)ccc(N)c1F] | 2-fluoro-4-((7-fluoro-1,6-dimethyl- 1H-benzo[d][1,2,3]triazol-5-yl)- oxy)-3-methylaniline | 305.1 (M + H) |
| I110 | 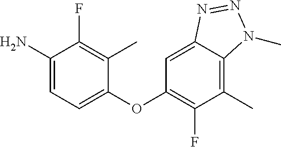 [SMILES: Cc1c(Oc2cc3nnn(C)c3c(C)c2F)ccc(N)c1F] | 2-fluoro-4-((6-fluoro-1,7-dimethyl- 1H-benzo[d][1,2,3]triazol-5-yl)- oxy)-3-methylaniline | 305.0 (M + H) |
| I111 | 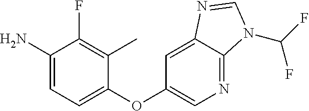 [SMILES: Cc1c(Oc2cnc3c(c2)ncn3C(F)F)ccc(N)c1F] | 4-((3-(difluoromethyl)-3H-imidazo [4,5-b]pyridin-6-yl)oxy)-2-fluoro- 3-methylaniline | 308.9 (M + H) |
| I112 | 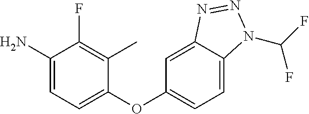 [SMILES: Cc1c(Oc2ccc3c(c2)nnn3C(F)F)ccc(N)c1F] | 4-((1-(difluoromethyl)-1H-benzo [d][1,2,3]triazol-5-yl)oxy)-2-fluoro- 3-methylaniline | 309.1 (M + H) |
| I113 | 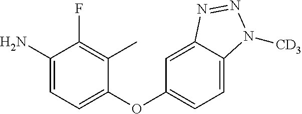 [SMILES: [2H]C([2H])([2H])n1nnc2cc(Oc3ccc(N)c(F)c3C)ccc21] | 2-fluoro-3-methyl-4-((1-(methyl- d3)-1H-benzo[d][1,2,3]triazol-5-yl)- oxy)aniline | 276.1 (M + H) |
| I114 | 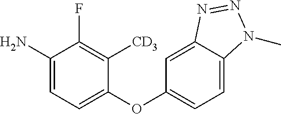 [SMILES: [2H]C([2H])([2H])c1c(Oc2ccc3c(c2)nnn3C)ccc(N)c1F] | 2-fluoro-3-(methyl-d3)-4-((1-meth- yl-1H-benzo[d][1,2,3]triazol-5-yl)- oxy)aniline | 276.1 (M + H) |
| I115 | 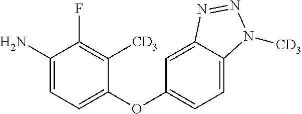 [SMILES: [2H]C([2H])([2H])c1c(Oc2ccc3c(c2)nnn3C([2H])([2H])[2H])ccc(N)c1F] | 2-fluoro-3-(methyl-d3)-4-((1-(meth- yl-d3)-1H-benzo[d][1,2,3]triazol-5- yl)oxy)aniline | |

### Example 32 (I116)

### 3-ethyl-4-((1-methyl-1H-benzo[d][1,2,3]triazol-5-yl)oxy)aniline

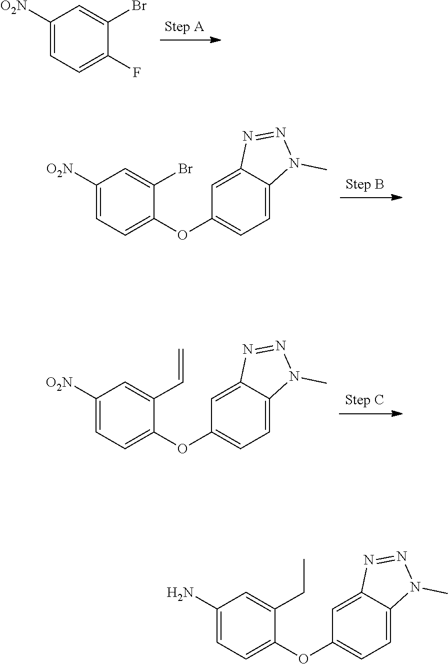 [SMILES: O=[N+]([O-])c1ccc(F)c(Br)c1 ; Cn1nnc2cc(Oc3ccc([N+](=O)[O-])cc3Br)ccc21 ; C=Cc1cc([N+](=O)[O-])ccc1Oc1ccc2c(c1)nnn2C ; CCc1cc(N)ccc1Oc1ccc2c(c1)nnn2C]

Step A. 5-(2-bromo-4-nitrophenoxy)-1-methyl-1H-benzo[d][1,2,3]triazole. To a solution of 2-bromo-1-fluoro-4-nitro-benzene (1.00 g, 4.55 mmol) in DMF (10 mL) were added K₂CO₃ (1.26 g, 9.09 mmol) and 1-methylbenzotriazol-5-ol (0.68 g, 4.55 mmol). The mixture was stirred at 25° C. for 2 h. The reaction was quenched with H₂O (50 mL) and extracted with EtOAc (50 mL×3). The combined organic layers were washed with water (50 mL×2) and brine (50 mL), dried over anhydrous sodium sulfate, filtered, and concentrated under reduced pressure. The crude residue was purified by silica gel column chromatography (0% to 25% EtOAc/PET) to afford 5-(2-bromo-4-nitro-phenoxy)-1-methyl-benzotriazole 3 (800 mg, 50% yield) as a brown solid. LCMS (MM-ES+APCI, Pos): m/z 349.0, 351.0 (M+H).

Step B. 1-methyl-5-(4-nitro-2-vinylphenoxy)-1H-benzo[d][1,2,3]triazole. To a solution of 5-(2-bromo-4-nitro-phenoxy)-1-methyl-benzotriazole (700 mg, 2.00 mmol) in 1,4-dioxane (10 mL) and water (2 mL) were added Pd(dppf)C₁₂ (145 mg, 0.20 mmol), Na₂CO₃ (425 mg, 4.01 mmol) and potassium vinyltrifluoroborate (322 mg, 2.41 mmol). The reaction was stirred at 110° C. for 2 h. The mixture was concentrated under vacuum and the crude residue was purified by silica gel column chromatography (0% to 25% EtOAc/PET) to provide 1-methyl-5-(4-nitro-2-vinyl-phenoxy)benzotriazole (500 mg, 79% yield) as a yellow solid. LCMS (MM-ES+APCI, Pos): m/z 297.2 (M+H).

Step C. 3-ethyl-4-((1-methyl-1H-benzo[d][1,2,3]triazol-5-yl)oxy)aniline. To a solution of 1-methyl-5-(4-nitro-2-vinyl-phenoxy)benzotriazole (500 mg, 1.57 mmol) in MeOH was added 10% Pd/C (1 g). The mixture was degassed and purged with H₂ three times. The reaction was stirred at 20° C. for 30 min and filtered through Celite eluting with MeOH (30 mL). The filtrate was concentrated and the residue was purified via silica gel column chromatography (1% to 30% EtOAc/PET) to give 3-ethyl-4-(1-methylbenzotriazol-5-yl)oxy-aniline 5 (230 mg, 52% yield) as a colorless oil. LCMS (MM-ES+APCI, Pos): m/z 269.3 (M+H).

### Example 33 (I117)

### 4-([1,2,4]triazolo[1,5-c]pyrimidin-7-yloxy)-3-methylaniline

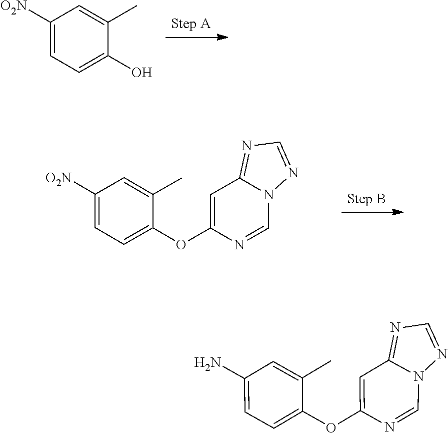 [SMILES: Cc1cc([N+](=O)[O-])ccc1O ; Cc1cc([N+](=O)[O-])ccc1Oc1cc2ncnn2cn1 ; Cc1cc(N)ccc1Oc1cc2ncnn2cn1]

Step A. 7-(2-methyl-4-nitrophenoxy)-[1,2,4]triazolo[1,5-c]pyrimidine. A solution of 2-methyl-4-nitrophenol (5.94 g, 38.8 mmol), 7-chloro-[1,2,4]triazolo[1,5-c]pyrimidine (5.00 g, 32.3 mmol), and K₂CO₃ (11.2 g, 80.9 mmol) in MeCN (50 mL) was heated at 80° C. for 3 h. The mixture was concentrated in vacuo, diluted with water (50 mL), and extracted with DCM (25 mL). The organic phase was washed with 2M aq. NaOH (25 mL), dried over anhydrous MgSO₄, and filtered. Concentrating the product to dryness afforded 7-(2-methyl-4-nitrophenoxy)-[1,2,4]triazolo[1,5-c]pyrimidine (2.87 g, 31% yield) as a yellow solid.

Step B. 4-([1,2,4]triazolo[1,5-c]pyrimidin-7-yloxy)-3-methylaniline. To a solution of 7-(2-methyl-4-nitrophenoxy)-[1,2,4]triazolo[1,5-c]pyrimidine (2.00 g, 7.37 mmol) in EtOH (30 mL) and water (10 mL) were added Fe (1.24 g, 22.1 mmol) and NH₄Cl (1.97 g, 36.9 mmol). The reaction was heated at 60° C. for 3 h, and cooled to ambient temperature. The mixture was filtered over Celite, washed with EtOH (10 mL), and volatiles were removed in vacuo. Water (20 mL) was added, the product was extracted with EtOAc (10 mL×3), and the combined organics were concentrated under reduced pressure. The crude material was purified via silica gel column chromatography (25% to 60% EtOAc/Heptanes) to afford 4-([1,2,4]triazolo[1,5-c]pyrimidin-7-yloxy)-3-methylaniline (0.92 g, 51% yield) as a yellow solid. LCMS (MM-ES+APCI, Pos): m/z 242.3 (M+H).

### Example 34 (I29)

### N-(4-((7-fluoro-1-methyl-H-benzo[d]1[1,2,3]triazol-5-yl)oxy)-3-methylphenyl)-6-(methylthio)pyrimido[5,4-d]pyrimidin-4-amine

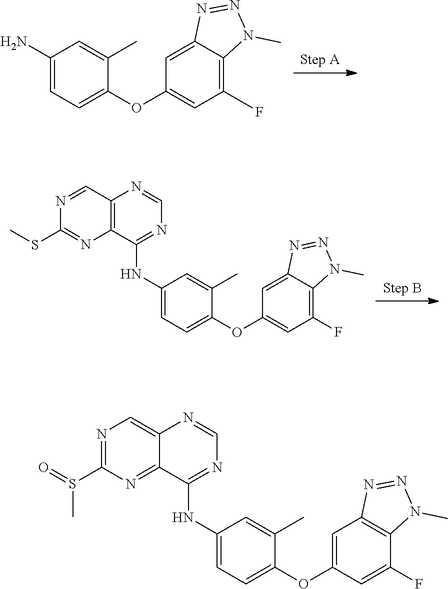

Step A. N-(4-((7-fluoro-1-methyl-1H-benzo[d][1,2,3]triazol-5-yl)oxy)-3-methylphenyl)-6-(methylthio)pyrimido[5,4-d]pyrimidin-4-amine. To a solution of 4-(7-fluoro-1-methyl-benzotriazol-5-yl)oxy-3-methyl-aniline (130 mg, 0.48 mmol) and 4-chloro-6-methylsulfanyl-pyrimido[5,4-d]pyrimidine (203 mg, 0.96 mmol) in IPA (5 mL) was added DIPEA (250 μL, 1.43 mmol). The mixture was stirred at 25° C. for 12 h and concentrated under reduced pressure. The residue was triturated with MeCN (5 mL) at 25° C. for 30 min. Filtration afforded N-[4-(7-fluoro-1-methyl-benzotriazol-5-yl)oxy-3-methyl-phenyl]-6-methylsulfanyl-pyrimido[5,4-d]pyrimidin-4-amine (190 mg, 84% yield) as a gray solid. LCMS (MM-ES+APCI, Pos): m/z 449.2 (M+H).

Step B. N-(4-((7-fluoro-1-methyl-1H-benzo[d][1,2,3]triazol-5-yl)oxy)-3-methylphenyl)-6-(methylsulfinyl)pyrimido[5,4-d]pyrimidin-4-amine. To a solution of N-[4-(7-fluoro-1-methyl-benzotriazol-5-yl)oxy-3-methyl-phenyl]-6-methylsulfanyl-pyrimido[5,4-d]pyrimidin-4-amine (270 mg, 0.60 mmol) in MeCN (3 mL) and H₂O (3 mL) was added Oxone (242 mg, 1.44 mmol). The reaction mixture was stirred at 50° C. for 1.5 h, diluted with sat. aq. NaHCO₃ (15 mL), and extracted with DCM (15 mL×3). The combined organic layers were washed with brine (20 mL), dried over anhydrous Na₂SO₄, filtered, and concentrated in vacuo to provide N-[4-(7-fluoro-1-methyl-benzotriazol-5-yl)oxy-3-methyl-phenyl]-6-methylsulfinyl-pyrimido[5,4-d]pyrimidin-4-amine (190 mg, 68% yield) as a yellow gum. LCMS (MM-ES+APCI, Pos): m/z 465.1 (M+H).

Intermediates I29-I46, I118-I144 in Table 3 below were prepared according to Example 34 Steps A to B. Step A can be accomplished using DIPEA, TEA, or Cs₂CO₃ as base. Depending on the substrate, either the sulfoxide or sulfone was isolated. Both oxidation states are competent in subsequent reactions.

TABLE 3
A list of intermediate compounds I29-I46, I117-I144

| | | | MS |
|---|---|---|---|
| Int. | | | API |
| ID | Structure | Chemical Name | (m/z) |
| I29 | 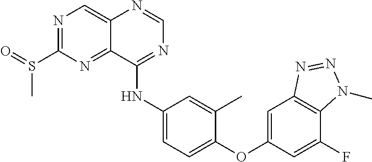 [SMILES: Cc1cc(Nc2ncnc3cnc(S(C)=O)nc23)ccc1Oc1cc(F)c2c(c1)nnn2C] | N-(4-((7-fluoro-1-methyl-1H- benzo[d][1,2,3]triazol-5-yl)- oxy)-3-methylphenyl)-6-(meth- ylthio)pyrimido[5,4-d]pyrim- idin-4-amine | 465.1 (M + H) |
| I30 | 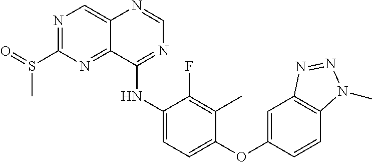 [SMILES: Cc1c(Oc2ccc3c(c2)nnn3C)ccc(Nc2ncnc3cnc(S(C)=O)nc23)c1F] | N-(2-fluoro-3-methyl-4-((1- methyl-1H-benzo[d][1,2,3] triazol-5-yl)oxy)phenyl)-6- (methylsulfinyl)pyrimido[5,4- d]pyrimidin-4-amine | 465.0 (M + H) |
| I31 | 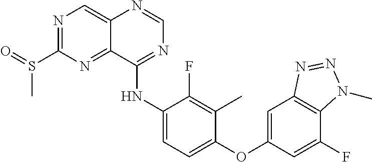 [SMILES: Cc1c(Oc2cc(F)c3c(c2)nnn3C)ccc(Nc2ncnc3cnc(S(C)=O)nc23)c1F] | N-(2-fluoro-4-((7-fluoro-1- methyl-1H-benzo[d][1,2,3] triazol-5-yl)oxy)-3-methyl- phenyl)-6-(methylsulfinyl)- pyrimido[5,4-d]pyrimidin-4- amine | 483.3 (M + H) |
| I32 | 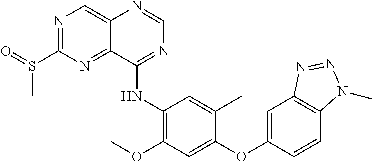 [SMILES: COc1cc(Oc2ccc3c(c2)nnn3C)c(C)cc1Nc1ncnc2cnc(S(C)=O)nc12] | N-(2-methoxy-5-methyl-4-((1- methyl-1H-benzo[d][1,2,3] triazol-5-yl)oxy)phenyl)-6- (methylsulfinyl)pyrimido[5,4- d]pyrimidin-4-amine | 477.1 (M + H) |
| I33 | 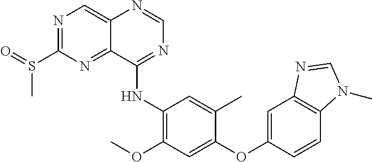 [SMILES: COc1cc(Oc2ccc3c(c2)ncn3C)c(C)cc1Nc1ncnc2cnc(S(C)=O)nc12] | N-(2-methoxy-5-methyl-4-((1- methyl-1H-benzo[d]imidazol- 5-yl)oxy)phenyl)-6-(methyl- sulfinyl)pyrimido[5,4-d]pyr- imidin-4-amine | 476.2 (M + H) |
| I34 | 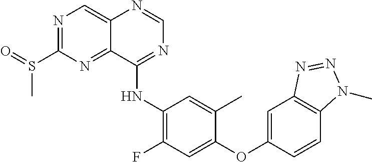 [SMILES: Cc1cc(Nc2ncnc3cnc(S(C)=O)nc23)c(F)cc1Oc1ccc2c(c1)nnn2C] | N-(2-fluoro-5-methyl-4-((1- methyl-1H-benzo[d][1,2,3] triazol-5-yl)oxy)phenyl)-6- (methylsulfinyl)pyrimido[5,4- d]pyrimidin-4-amine | 465.0 (M + H) |
| I35 | 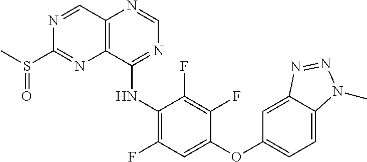 | 6-(methylsulfinyl)-N-(2,3,6- trifluoro-4-((1-methyl-1H- benzo[d][1,2,3]triazol-5-yl)- oxy)phenyl)pyrimido[5,4-d]- pyrimidin-4-amine | 487.1 (M + H) |
| I36 | 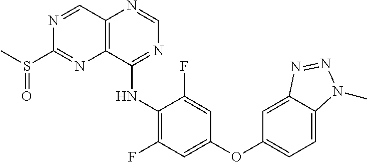 | N-(2,6-difluoro-4-((1-methyl- 1H-benzo[d][1,2,3]triazol-5- yl)oxy)phenyl)-6-(methylsul- finyl)pyrimido[5,4-d]pyrim- idin-4-amine | 469.1 (M + H) |
| I37 | 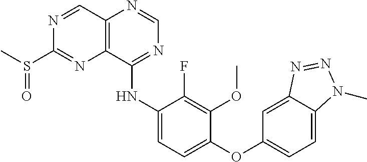 | N-(2-fluoro-3-methoxy-4-((1- methyl-1H-benzo[d][1,2,3] triazol-5-yl)oxy)phenyl)-6- (methylsulfinyl)pyrimido[5,4- d]pyrimidin-4-amine | 481.1 (M + H) |
| I38 | 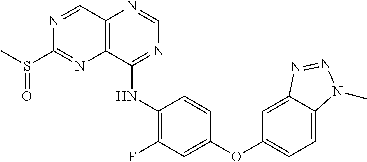 | N-(2-fluoro-4-((1-methyl-1H- benzo[d][1,2,3]triazol-5-yl)- oxy)phenyl)-6-(methylsulfin- yl)pyrimido[5,4-d]pyrimidin- 4-amine | 451.0 (M + H) |
| I39 | 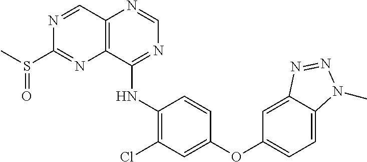 | N-(2-chloro-4-((1-methyl-1H- benzo[d][1,2,3]triazol-5-yl)- oxy)phenyl)-6-(methylsulfin- yl)pyrimido[5,4-d]pyrimidin- 4-amine | 467.2 (M + H) |
| 140 | 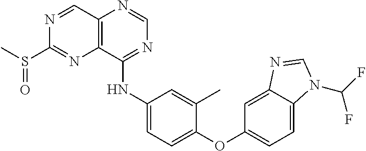 [SMILES: Cc1cc(Nc2ncnc3cnc(S(C)=O)nc23)ccc1Oc1ccc2c(c1)ncn2C(F)F] | N-(4-((1-(difluoromethyl)-1H- benzo[d]imidazol-5-yl)oxy)-3- methylphenyl)-6-(methylsul- finyl)pyrimido[5,4-d]pyrim- idin-4-amine | 482.3 (M + H) |
| I41 | 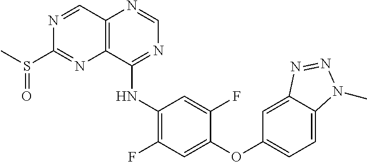 | N-(2,5-difluoro-4-((1-methyl- 1H-benzo[d][1,2,3]triazol-5- yl)oxy)phenyl)-6-(methylsul- finyl)pyrimido[5,4-d]pyrim- idin-4-amine | 469.1 (M + H) |
| I42 | 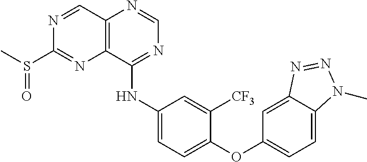 | N-(4-((1-methyl-1H-benzo[d] [1,2,3]triazol-5-yl)oxy)-3- (trifluoromethyl)phenyl)-6- (methylsulfinyl)pyrimido[5,4- d]pyrimidin-4-amine | 501.1 (M + H) |
| I43 | 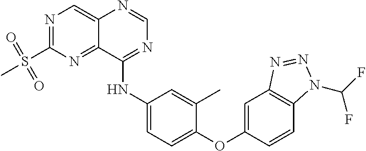 [SMILES: Cc1cc(Nc2ncnc3cnc(S(C)(=O)=O)nc23)ccc1Oc1ccc2c(c1)nnn2C(F)F] | N-(4-((1-(difluoromethyl)-1H- benzo[d][1,2,3]triazol-5-yl)- oxy)-3-methylphenyl)-6-(meth- ylsulfonyl)pyrimido[5,4-d] pyrimidin-4-amine | 499.1 (M + H) |
| I44 | 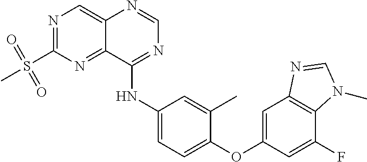 | N-(4-((7-fluoro-1-methyl-1H- benzo[d]imidazol-5-yl)oxy)-3- methylphenyl)-6-(methylsul- fonyl)pyrimido[5,4-d]pyrim- idin-4-amine | 480.2 (M + H) |
| I45 | 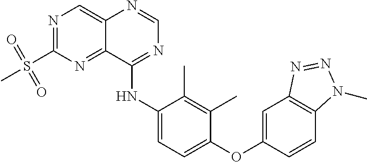 | N-(2,3-dimethyl-4-((1-methyl- 1H-benzo[d][1,2,3]triazol-5- yl)oxy)phenyl)-6-(methylsul- fonyl)pyrimido[5,4-d]pyrim- idin-4-amine | 477.0 (M + H) |
| I46 | 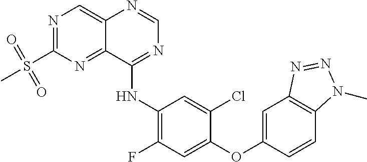 | N-(5-chloro-2-fluoro-4-((1- methyl-1H-benzo[d][1,2,3] triazol-5-yl)oxy)phenyl)-6- (methylsulfonyl)pyrimido[5,4- d]pyrimidin-4-amine | 501.3 (M + H) |
| I118 | 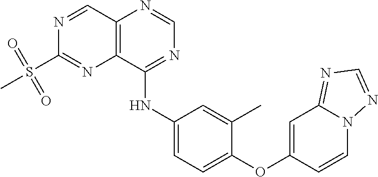 [SMILES: Cc1cc(Nc2ncnc3cnc(S(C)(=O)=O)nc23)ccc1Oc1ccn2ncnc2c1] | N-(4-([1,2,4]triazolo[1,5-a] pyridin-7-yloxy)-3-methyl- phenyl)-6-methylsulfonyl)- pyrimido[5,4-d]pyrimidin-4- amine | 449.2 (M + H) |
| I119 | 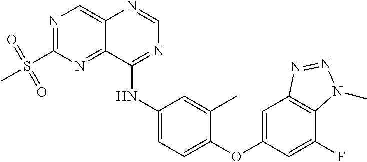 | N-(4-((7-fluoro-1-methyl-1H- benzo[d][1,2,3]triazol-5-yl)- oxy)-3-methylphenyl)-6- (methylsulfonyl)pyrimido[5,4- d]pyrimidin-4-amine | 481.0 (M + H) |
| I120 | 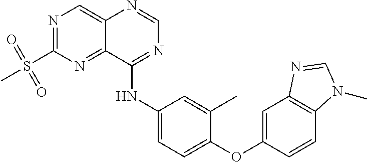 | N-(3-methyl-4-((1-methyl-1H- benzo[d]imidazol-5-yl)oxy)- phenyl)-6-(methylsulfonyl)- pyrimido[5,4-d]pyrimidin-4- amine | 462.1 (M + H) |
| I121 | 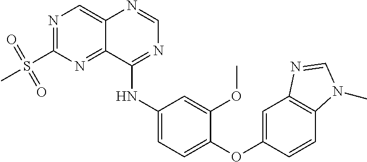 | N-(3-methoxy-4-((1-methyl- 1H-benzo[d]imidazol-5-yl)- oxy)phenyl)-6-methylsulfon- yl)pyrimido[5,4-d]pyrimidin- 4-amine | 478.1 (M + H) |
| I122 | 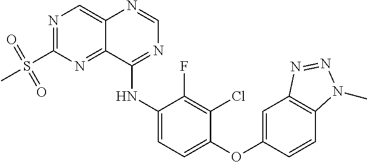 | N-(3-chloro-2-fluoro-4-((1- methyl-1H-benzo[d][1,2,3] triazol-5-yl)oxy)phenyl)-6- (methylsulfonyl)pyrimido[5,4- d]pyrimidin-4-amine | 501.0 (M + H) |
| I123 | 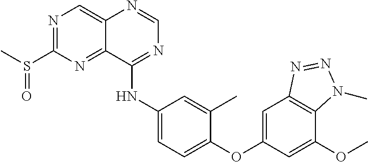 | N-(4-((7-methoxy-1-methyl- 1H-benzo[d][1,2,3]triazol-5- yl)oxy)-3-methylphenyl)-6- (methylsulfinyl)pyrimido[5,4- d]pyrimidin-4-amine | 477.2 (M + H) |
| I124 | 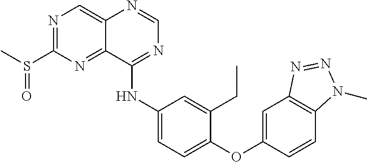 | N-(3-ethyl-4-((1-methyl-1H- benzo[d][1,2,3]triazol-5-yl)- oxy)phenyl)-6-(methylsulfin- yl)pyrimido[5,4-d]pyrimidin- 4-amine | 461.1 (M + H) |
| I125 | 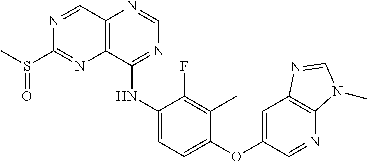 | N-(2-fluoro-3-methyl-4-((3- methyl-3H-imidazo[4,5-b]pyr- idin-6-yl)oxy)phenyl)-6-(meth- ylsulfinyl)pyrimido[5,4-d]pyr- imidin-4-amine | 465.3 (M + H) |
| I126 | 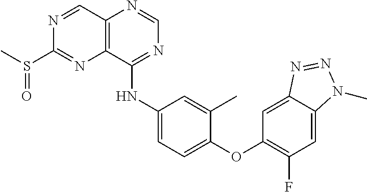 | N-(4-((6-fluoro-1-methyl-1H- benzo[d][1,2,3]triazol-5-yl)- oxy)-3-methylphenyl)-6- (methylsulfonyl)pyrimido[5,4- d]pyrimidin-4-amine | 465.1 (M + H) |
| I127 | 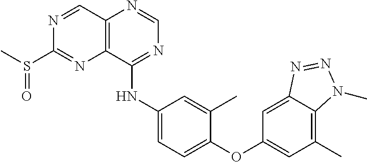 | N-(4-((1,7-dimethyl-1H-benzo [d][1,2,3]triazol-5-yl)oxy)-3- methylphenyl)-6-(methylsul- finyl)pyrimido[5,4-d]pyrim- idin-4-amine | 461.2 (M + H) |
| I128 | 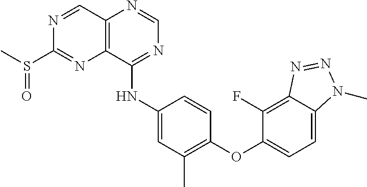 | N-(4-((4-fluoro-1-methyl-1H- benzo[d][1,2,3]triazol-5-yl)- oxy)-3-methylphenyl)-6- (methylsulfonyl)pyrimido[5,4- d]pyrimidin-4-amine | 465.2 (M + H) |
| I129 | 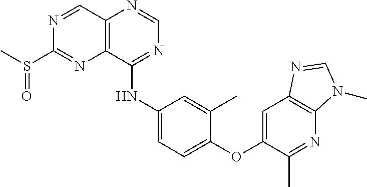 | N-(4-((3,5-dimethyl-3H-imid- azo[4,5-b]pyridin-6-yl)oxy)-3- methylphenyl)-6-(methylsul- finyl)pyrimido[5,4-d]pyrim- idin-4-amine | 461.1 (M + H) |
| I130 | 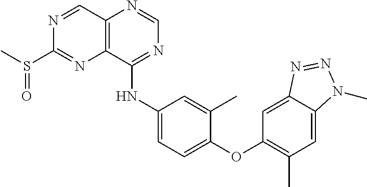 | N-(4-((1,6-dimethyl-1H-benzo [d][1,2,3]triazol-5-yl)oxy)-3- methylphenyl)-6-(methylsul- finyl)pyrimido[5,4-d]pyrim- idin-4-amine | 461.2 (M + H) |
| I131 | 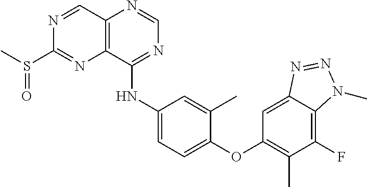 | N-(4-((7-fluoro-1,6-dimethyl- 1H-benzo[d][1,2,3]triazol-5- yl)oxy)-3-methylphenyl)-6- (methylsulfinyl)pyrimido[5,4- d]pyrimidin-4-amine | 479.2 (M + H) |
| I132 | 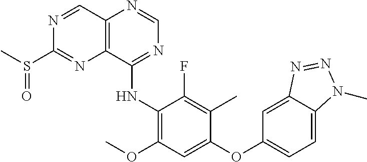 | N-(2-fluoro-6-methoxy-3- methyl-4-((1-methyl-1H- benzo[d][1,2,3]triazol-5-yl)- oxy)phenyl)-6-(methylsulfin- yl)pyrimido[5,4-d]pyrimidin- 4-amine | 495.1 (M + H) |
| I133 | 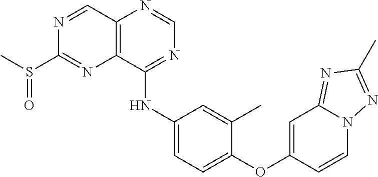 | N-(3-methyl-4-((2-methyl- [1,2,4]triazolo[1,5-a]pyridin- 7-yl)oxy)phenyl)-6-(methyl- sulfinyl)pyrimido[5,4-d]pyr- imidin-4-amine | 447.2 (M + H) |
| I134 | 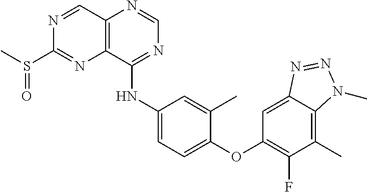 | N-(4-((6-fluoro-1,7-dimethyl- 1H-benzo[d][1,2,3]triazol-5- yl)oxy)-3-methylphenyl)-6- (methylsulfinyl)pyrimido[5,4- d]pyrimidin-4-amine | 479.1 (M + H) |
| I135 | 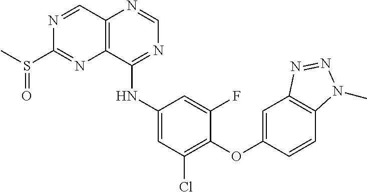 | N-(3-chloro-5-fluoro-4-((1- methyl-1H-benzo[d][1,2,3] triazol-5-yl)oxy)phenyl)-6- (methylsulfinyl)pyrimido[5,4- d]pyrimidin-4-amine | 485.2 (M + H) |
| I136 | 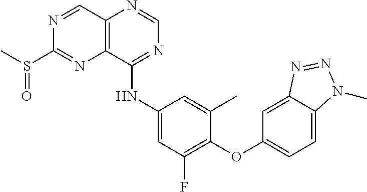 | N-(3-fluoro-5-methyl-4-((1- methyl-1H-benzo[d][1,2,3] triazol-5-yl)oxy)phenyl)-6- (methylsulfinyl)pyrimido[5,4- d]pyrimidin-4-amine | 465.2 (M + H) |
| I137 | 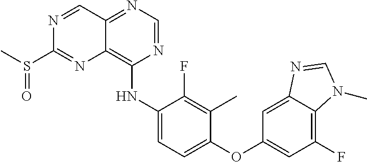 | N-(2-fluoro-4-((7-fluoro-1- methyl-1H-benzo[d]imidazol- 5-yl)oxy)-3-methylphenyl)-6- (methylsulfinyl)pyrimido[5,4- d]pyrimidin-4-amine | 482.3 (M + H) |
| I138 | 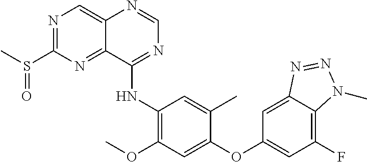 | N-(4-((7-fluoro-1-methyl-1H- benzo[d][1,2,3]triazol-5-yl)- oxy)-2-methoxy-5-methyl- phenyl)-6-(methylsulfinyl)- pyrimido[5,4-d]pyrimidin-4- amine | 495.1 (M + H) |
| I139 | 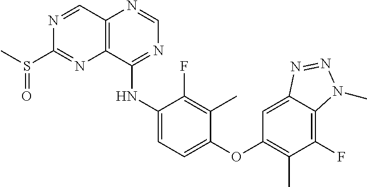 | N-(2-fluoro-4-((7-fluoro-1,6- dimethyl-1H-benzo[d][1,2,3] triazol-5-yl)oxy)-3-methyl- phenyl)-6-(methylsulfinyl)- pyrimido[5,4-d]pyrimidin-4- amine | 497.0 (M + H) |
| I140 | 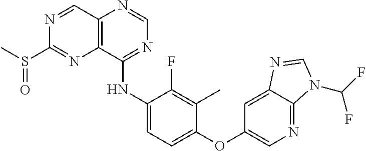 [SMILES: Cc1c(Oc2cnc3c(c2)ncn3C(F)F)ccc(Nc2ncnc3cnc(S(C)=O)nc23)c1F] | N-(4-((3-(difluoromethyl)-3H- imidazo[4,5-b]pyridin-6-yl)- oxy)-2-fluoro-3-methylphen- yl)-6-(methylsulfinyl)pyrim- ido[5,4-d]pyrimidin-4-amine | 500.9 (M + H) |
| I141 | 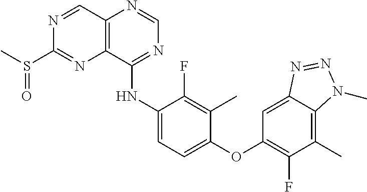 | N-(2-fluoro-4-((6-fluoro-1,7- dimethyl-1H-benzo[d][1,2,3] triazol-5-yl)oxy)-3-methyl- phenyl)-6-(methylsulfinyl)- pyrimido[5,4-d]pyrimidin-4- amine | 496.9 (M + H) |
| I142 | 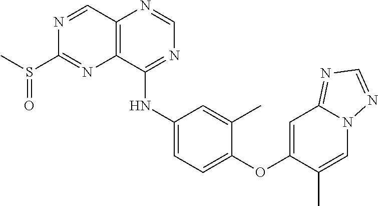 [SMILES: Cc1cc(Nc2ncnc3cnc(S(C)=O)nc23)ccc1Oc1cc2ncnn2cc1C] | N-(3-methyl-4-((6-methyl- [1,2,4]triazolo[1,5-a]pyridin- 7-yl)oxy)phenyl)-6-(methyl- sulfinyl)pyrimido[5,4-d]pyr- imidin-4-amine | 447.3 (M + H) |
| I143 | 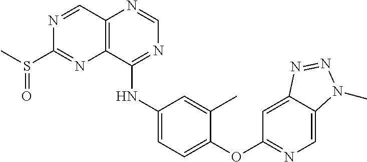 | N-(3-methyl-4-((3-methyl-3H- [1,2,3]triazolo[4,5-c]pyridin- 6-yl)oxy)phenyl)-6-(methyl- sulfinyl)pyrimido[5,4-d]pyr- imidin-4-amine | 448.1 (M + H) |
| I144 | 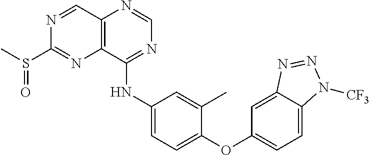 [SMILES: Cc1cc(Nc2ncnc3cnc(S(C)=O)nc23)ccc1Oc1ccc2c(c1)nnn2C(F)(F)F] | N-(3-methyl-4-((1-(trifluoro- methyl)-1H-benzo[d][1,2,3] triazol-5-yl)oxy)phenyl)-6- (methylsulfinyl)pyrimido[5,4- d]pyrimidin-4-amine | 501.1 (M + H) |

### Example 35 (I47)

### N-(3-methyl-4-((1-methyl-1H-benzo[d][1,2,3]triazol-5-yl)oxy)phenyl)-6-(methylsulfinyl)pyrimido[5,4-d]pyrimidin-4-amine

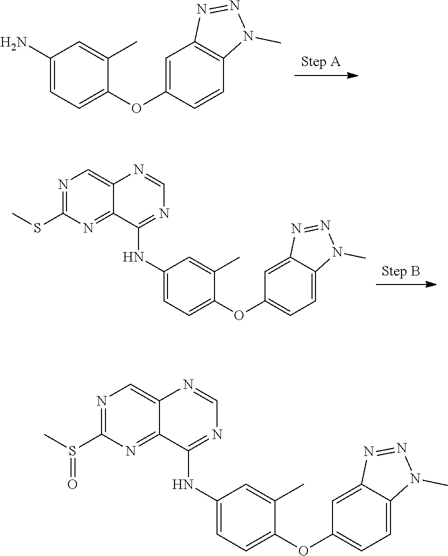

Step A. N-(3-methyl-4-((1-methyl-1H-benzo[d][1,2,3]triazol-5-yl)oxy)phenyl)-6-(methylthio)pyrimido[5,4-d]pyrimidin-4-amine. To a solution of 3-methyl-4-((1-methyl-1H-benzo[d][1,2,3]triazol-5-yl)oxy)aniline (21.0 g, 82.6 mmol) and 8-chloro-2-(methylthio)pyrimido[5,4-d]pyrimidine (26.3 g, 124 mmol) in DMF (413 mL) was added TEA (13.8 mL, 99.1 mmol). The reaction was heated at 45° C. for 2.5 h, cooled to 0° C., and water (500 mL) was added. After stirring 15 min, the mixture was filtered and the solid rinsed with water. The solids were collected and dried under vacuum at 40° C. to provide N-(3-methyl-4-((1-methyl-1H-benzo[d][1,2,3]triazol-5-yl)oxy)phenyl)-6-(methylthio)pyrimido[5,4-d]pyrimidin-4-amine (33.6 g, 95% yield). LCMS (MM-ES+APCI, Pos): m/z 431.2 (M+H).

Step B. N-(3-methyl-4-((1-methyl-1H-benzo[d][1,2,3]triazol-5-yl)oxy)phenyl)-6-(methylsulfinyl)pyrimido[5,4-d]pyrimidin-4-amine. A solution of N-(3-methyl-4-((1-methyl-1H-benzo[d][1,2,3]triazol-5-yl)oxy)phenyl)-6-(methylthio)pyrimido[5,4-d]pyrimidin-4-amine (51.9 g, 121 mmol) in DCM (603 mL) was cooled to 0° C. A solution of mCPBA (29.1 g, 127 mmol, 75% wt.) in DCM (300 mL) was added slowly over 2 h. The ice bath was removed and the reaction was stirred at 25° C. for 30 min. Sodium thiosulfate (1M, 1 L) was slowly added and the reaction was stirred for 10 min. MeOH (300 mL) was added slowly to solubilize solids and the layers were separated. The organics were washed with sat. aq. NaHCO₃ and brine, dried over Na₂SO₄, filtered and concentrated to give N-(3-methyl-4-((1-methyl-1H-benzo[d][1,2,3]triazol-5-yl)oxy)phenyl)-6-(methylsulfinyl)pyrimido[5,4-d]pyrimidin-4-amine (47.8 g, 89% yield). LCMS (MM-ES+APCI, Pos): m/z 447.2 (M+H).

### Example 36 (I48)

### N-(2-fluoro-3-methyl-4-((1-methyl-1H-benzo[d]imidazol-5-yl)oxy)phenyl)-6-(methylsulfinyl)pyrimido[5,4-d]pyrimidin-4-amine

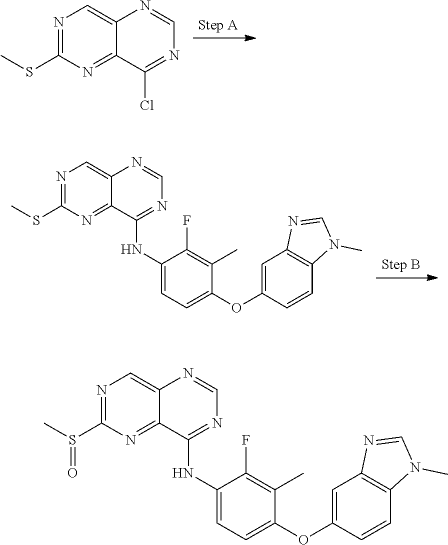

Step A. N-(2-fluoro-3-methyl-4-((1-methyl-1H-benzo[d]imidazol-5-yl)oxy)phenyl)-6-(methylthio)pyrimido[5,4-d]pyrimidin-4-amine. To a solution of 2-fluoro-3-methyl-4-(1-methylbenzimidazol-5-yl)oxy-aniline (250 mg, 0.92 mmol) and 4-chloro-6-methylsulfanyl-pyrimido[5,4-d]pyrimidine (600 mg, 2.82 mmol) in n-BuOH (5 mL) was added TFA (0.21 mL, 2.83 mmol). The mixture was stirred at 80° C. for 2 h and concentrated in vacuo. The crude material was purified by preparative silica gel TLC (10% MeOH/DCM) to afford N-[2-fluoro-3-methyl-4-(1-methylbenzimidazol-5-yl)oxy-phenyl]-6-methylsulfanyl-pyrimido[5,4-d]pyrimidin-4-amine (210 mg, 51% yield) as a yellow solid. LCMS (MM-ES+APCI, Pos): m/z 448.1 (M+H).

Step B. N-(2-fluoro-3-methyl-4-((1-methyl-1H-benzo[d]imidazol-5-yl)oxy)phenyl)-6-(methylsulfinyl)pyrimido[5,4-d]pyrimidin-4-amine. Prepared according to Example 34 Step B, substituting N-[2-fluoro-3-methyl-4-(1-methylbenzimidazol-5-yl)oxy-phenyl]-6-methylsulfanyl-pyrimido[5,4-d]pyrimidin-4-amine for N-[4-(7-fluoro-1-methyl-benzotriazol-5-yl)oxy-3-methyl-phenyl]-6-methylsulfanyl-pyrimido[5,4-d]pyrimidin-4-amine to give N-[2-fluoro-3-methyl-4-(1-methylbenzimidazol-5-yl)oxy-phenyl]-6-methylsulfinyl-pyrimido[5,4-d]pyrimidin-4-amine (150 mg, 72% yield) as a yellow solid. LCMS (MM-ES+APCI, Pos): m/z 464.0 (M+H).

Intermediates I48-I50, I145-I148 in Table 4 below were prepared according to Example 36 Steps A to B.

TABLE 4
A list of intermediate compounds I48-I50, I144-I147

| | | | MS |
|---|---|---|---|
| Int. | | | API |
| ID | Structure | Chemical Name | (m/z) |
| I48 | 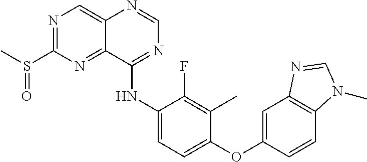 | N-(2-fluoro-3-methyl-4-((1- methyl-1H-benzo[d]imid- azol-5-yl)oxy)phenyl)-6- (methylsulfinyl)pyrimido [5,4-d]pyrimidin-4-amine | 464.0 |
| I49 | 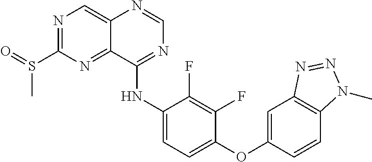 [SMILES: Cn1nnc2cc(Oc3ccc(Nc4ncnc5cnc(S(C)=O)nc45)c(F)c3F)ccc21] | N-(2,3-difluoro-4-((1-meth- yl-1H-benzo[d][1,2,3]triazol- 5-yl)oxy)phenyl)-6-(methyl- sulfinyl)pyrimido[5,4-d] pyrimidin-4-amine | 469.1 |
| I50 | 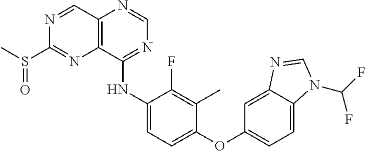 [SMILES: Cc1c(Oc2ccc3c(c2)ncn3C(F)F)ccc(Nc2ncnc3cnc(S(C)=O)nc23)c1F] | N-(4-((1-(difluoromethyl)- 1H-benzo[d]imidazol-5-yl)- oxy)-2-fluoro-3-methyl- phenyl)-6-(methylsulfinyl)- pyrimido[5,4-d]pyrimidin- 4-amine | 500.0 |
| I145 | 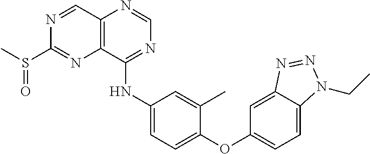 [SMILES: CCn1nnc2cc(Oc3ccc(Nc4ncnc5cnc(S(C)=O)nc45)cc3C)ccc21] | N-(4-((1-ethyl-1H-benzo[d] [1,2,3]triazol-5-yl)oxy)-3- methylphenyl)-6-(methyl- sulfinyl)pyrimido[5,4-d] pyrimidin-4-amine | 461.2 (M + H) |
| I146 | 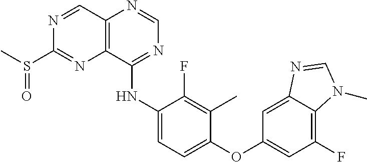 | N-(2-fluoro-4-((7-fluoro-1- methyl-1H-benzo[d]imid- azol-5-yl)oxy)-3-methyl- phenyl)-6-(methylsulfinyl)- pyrimido[5,4-d]pyrimidin- 4-amine | 482.1 (M + H) |
| I147 | 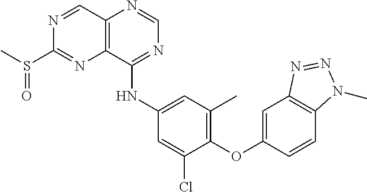 | N-(3-chloro-5-methyl-4-((1- methyl-1H-benzo[d][1,2,3] triazol-5-yl)oxy)phenyl)-6- (methylsulfinyl)pyrimido [5,4-d]pyrimidin-4-amine | 481.1 (M + H) |
| I148 | 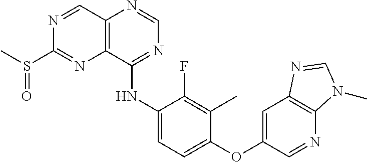 | N-(2-fluoro-3-methyl-4-((3- methyl-3H-imidazo[4,5-b] pyridin-6-yl)oxy)phenyl)-6- (methylsulfinyl)pyrimido [5,4-d]pyrimidin-4-amine | 465.3 (M + H) |

### Example 37 (I51)

### 6-chloro-N-(3-methyl-4-((1-methyl-H-benzo[d]i[1,2,3]triazol-5-yl)oxy)phenyl)pyrido[3,2-d]pyrimidin-4-amine

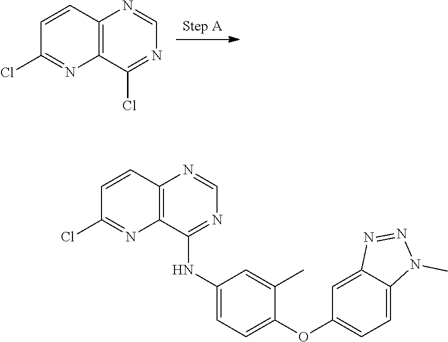 [SMILES: Clc1ccc2ncnc(Cl)c2n1 ; Cc1cc(Nc2ncnc3ccc(Cl)nc23)ccc1Oc1ccc2c(c1)nnn2C]

4,6-dichloropyrido[3,2-d]pyrimidine (2.36 g, 11.8 mmol) and 3-methyl-4-((1-methyl-1H-benzo[d][1,2,3]triazol-5-yl)oxy)aniline (3.00 g, 11.8 mmol) were suspended in IPA (29.5 mL) and heated to 85° C. for 40 min. The reaction was cooled to ambient temperature, the slurry was vacuum filtered, and the precipitate was washed with MTBE. The filtrate was concentrated in vacuo and the solid was washed with MTBE. The two crops of solid were combined to provide 6-chloro-N-(3-methyl-4-((1-methyl-1H-benzo[d][1,2,3]triazol-5-yl)oxy)phenyl)pyrido[3,2-d]pyrimidin-4-amine (5.16 g, quant. yield) as a fine yellow powder. LCMS (MM-ES+APCI, Pos): m/z 418.1 (M+H).

### Example 38 (I52)

### 6-chloro-N-(2-fluoro-4-((7-fluoro-1-methyl-H-benzo[d][1,2,3]triazol-5-yl)oxy)-3-methylphenyl)pyrido[3,2-d]pyrimidin-4-amine

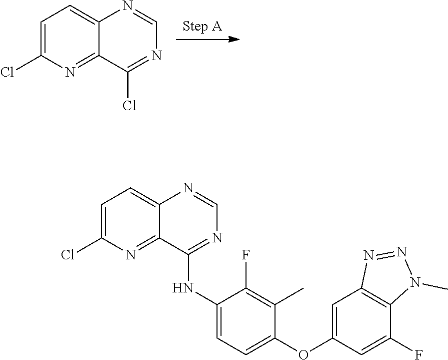 [SMILES: Clc1ccc2ncnc(Cl)c2n1 ; Cc1c(Oc2cc(F)c3c(c2)nnn3C)ccc(Nc2ncnc3ccc(Cl)nc23)c1F]

Step A. 6-chloro-N-(2-fluoro-4-((7-fluoro-1-methyl-1H-benzo[d][1,2,3]triazol-5-yl)oxy)-3-methylphenyl)pyrido[3,2-d]pyrimidin-4-amine. To a solution of 4,6-dichloropyrido[3,2-d]pyrimidine (55 mg, 0.28 mmol) and 2-fluoro-4-(7-fluoro-1-methyl-benzotriazol-5-yl)oxy-3-methyl-aniline (80 mg, 0.28 mmol) in IPA (5 mL) was added DIPEA (0.14 mL, 0.83 mmol). The mixture was stirred at 80° C. for 1 h and concentrated in vacuo. The crude material was purified by slurry in EtOAc (5 mL) at 25° C. for 10 m. After filtration, the solid was dried to provide 6-chloro-N-[2-fluoro-4-(7-fluoro-1-methyl-benzotriazol-5-yl)oxy-3-methyl-phenyl]pyridoy[3,2-d]pyrimidin-4-amine (135 mg, 79% yield) as a yellow solid. LCMS (MM-ES+APCI, Pos): m/z 454.3 (M+H).

Intermediates I52, I149-I164 in Table 13 below were prepared according to Example 38 Step A.

TABLE 13
A list of intermediate compounds I52, I149-I164

| | | | MS |
|---|---|---|---|
| Int. | | | API |
| ID | Structure | Chemical Name | (m/z) |
| I52 | 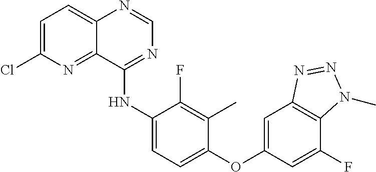 [SMILES: Cc1c(Oc2cc(F)c3c(c2)nnn3C)ccc(Nc2ncnc3ccc(Cl)nc23)c1F] | 6-chloro-N-(2-fluoro-4-((7-fluoro-1- methyl-1H-benzo[d][1,2,3]triazol-5- yl)oxy)-3-methylphenyl)pyrido[3,2- d]pyrimidin-4-amine | 454.3 (M + H) |
| I149 | 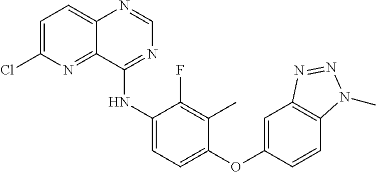 [SMILES: Cc1c(Oc2ccc3c(c2)nnn3C)ccc(Nc2ncnc3ccc(Cl)nc23)c1F] | 6-chloro-N-(2-fluoro-3-methyl-4- ((1-methyl-1H-benzo[d][1,2,3]tri- azol-5-yl)oxy)phenyl)pyrido[3,2- d]pyrimidin-4-amine | 436.2 (M + H) |
| I150 | 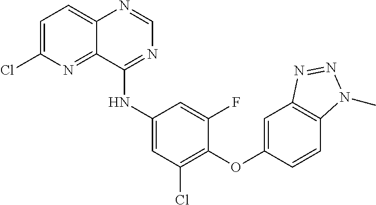 [SMILES: Cn1nnc2cc(Oc3c(F)cc(Nc4ncnc5ccc(Cl)nc45)cc3Cl)ccc21] | 6-chloro-N-(3-chloro-5-fluoro-4-((1- methyl-1H-benzo[d][1,2,3]triazol-5- yl)oxy)phenyl)pyrido[3,2-d]pyrim- idin-4-amine | 456.1 (M + H) |
| I151 | 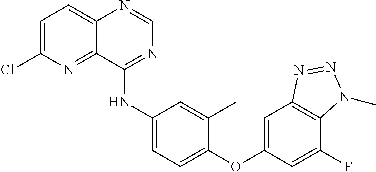 [SMILES: Cc1cc(Nc2ncnc3ccc(Cl)nc23)ccc1Oc1cc(F)c2c(c1)nnn2C] | 6-chloro-N-(4-((7-fluoro-1-methyl- 1H-benzo[d][1,2,3]triazol-5-yl)oxy)- 3-methylphenyl)pyrido[3,2-d]pyr- imidin-4-amine | 436.1 (M + H) |
| I152 | 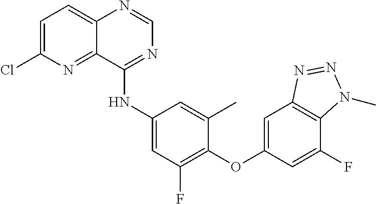 [SMILES: Cc1cc(Nc2ncnc3ccc(Cl)nc23)cc(F)c1Oc1cc(F)c2c(c1)nnn2C] | 6-chloro-N-(3-fluoro-4-((7-fluoro-1- methyl-1H-benzo[d][1,2,3]triazol-5- yl)oxy)-5-methylphenyl)pyrido[3,2- d]pyrimidin-4-amine | 436.2 (M + H) |
| I153 | 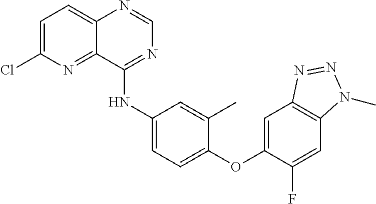 [SMILES: Cc1cc(Nc2ncnc3ccc(Cl)nc23)ccc1Oc1cc2nnn(C)c2cc1F] | 6-chloro-N-(4-((6-fluoro-1-methyl- 1H-benzo[d][1,2,3]triazol-5-yl)- oxy)-3-methylphenyl)pyrido[3,2-d] pyrimidin-4-amine | 436.2 (M + H) |
| I154 | 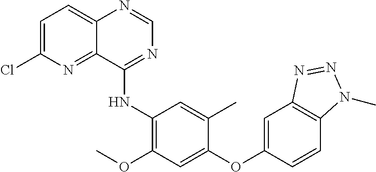 [SMILES: COc1cc(Oc2ccc3c(c2)nnn3C)c(C)cc1Nc1ncnc2ccc(Cl)nc12] | 6-chloro-N-(2-methoxy-5-methyl-4- ((1-methyl-1H-benzo[d][1,2,3]tri- azol-5-yl)oxy)phenyl)pyrido[3,2-d] pyrimidin-4-amine | 448.1 (M + H) |
| I155 | 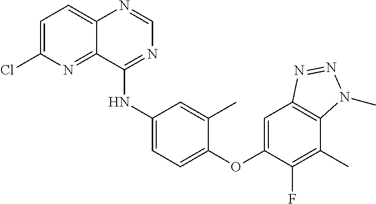 [SMILES: Cc1cc(Nc2ncnc3ccc(Cl)nc23)ccc1Oc1cc2nnn(C)c2c(C)c1F] | 6-chloro-N-(4-((6-fluoro-1,7-dimeth- yl-1H-benzo[d][1,2,3]triazol-5-yl)- oxy)-3-methylphenyl)pyrido[3,2-d] pyrimidin-4-amine | 450.2 (M + H) |
| I156 | 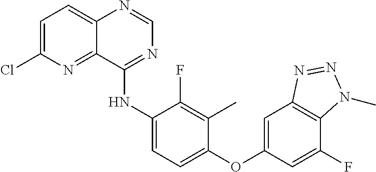 [SMILES: Cc1c(Oc2cc(F)c3c(c2)nnn3C)ccc(Nc2ncnc3ccc(Cl)nc23)c1F] | 6-chloro-N-(2-fluoro-4-((7-fluoro-1- methyl-1H-benzo[d][1,2,3]triazol-5- yl)oxy)-3-methylphenyl)pyrido[3,2- d]pyrimidin-4-amine | 454.1 (M + H) |
| I157 | 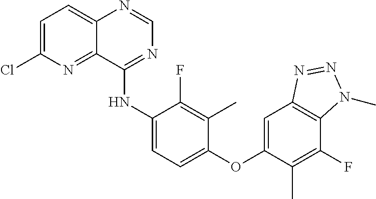 [SMILES: Cc1c(Oc2cc3nnn(C)c3c(F)c2C)ccc(Nc2ncnc3ccc(Cl)nc23)c1F] | 6-chloro-N-(2-fluoro-4-((7-fluoro- 1,6-dimethyl-1H-benzo[d][1,2,3] triazol-5-yl)oxy)-3-methylphenyl)- pyrido[3,2-d]pyrimidin-4-amine | 468.0 (M + H) |
| I158 | 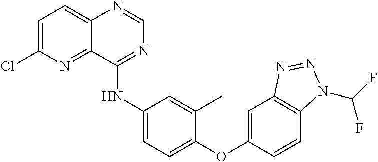 [SMILES: Cc1cc(Nc2ncnc3ccc(Cl)nc23)ccc1Oc1ccc2c(c1)nnn2C(F)F] | 6-chloro-N-(4-((1-(difluoromethyl)- 1H-benzo[d][1,2,3]triazol-5-yl)oxy)- 3-methylphenyl)pyrido[3,2-d]pyr- imidin-4-amine | 454.1 (M + H) |
| I159 | 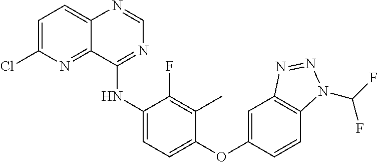 [SMILES: Cc1c(Oc2ccc3c(c2)nnn3C(F)F)ccc(Nc2ncnc3ccc(Cl)nc23)c1F] | 6-chloro-N-(4-((1-(difluoromethyl)- 1H-benzo[d][1,2,3]triazol-5-yl)oxy)- 2-fluoro-3-methylphenyl)pyrido[3,2- d]pyrimidin-4-amine | 472.1 (M + H) |
| I160 | 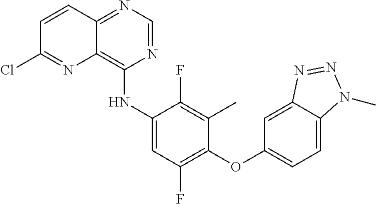 [SMILES: Cc1c(F)c(Nc2ncnc3ccc(Cl)nc23)cc(F)c1Oc1ccc2c(c1)nnn2C] | 6-chloro-N-(2,5-difluoro-3-methyl- 4-((1-methyl-1H-benzo[d][1,2,3] triazol-5-yl)oxy)phenyl)pyrido[3,2- d]pyrimidin-4-amine | 454.1 (M + H) |
| I161 | 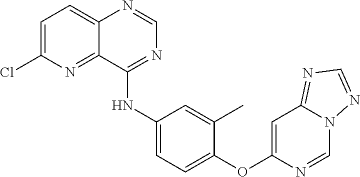 [SMILES: Cc1cc(Nc2ncnc3ccc(Cl)nc23)ccc1Oc1cc2ncnn2cn1] | N-(4-([1,2,4]triazolo[1,5-c]pyrim- idin-7-yloxy)-3-methylphenyl)-6- chloropyrido[3,2-d]pyrimidin-4- amine | 405.1 (M + H) |
| I162 | 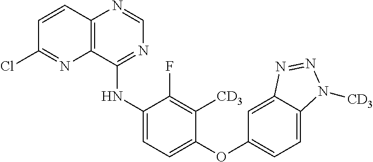 [SMILES: [2H]C([2H])([2H])c1c(Oc2ccc3c(c2)nnn3C([2H])([2H])[2H])ccc(Nc2ncnc3ccc(Cl)nc23)c1F] | 6-chloro-N-(2-fluoro-3-(methyl-d3)- 4-((1-(methyl-d3)-1H-benzo[d][1,2,3] triazol-5-yl)oxy)phenyl)pyrido[3,2- d]pyrimidin-4-amine | |
| I163 | 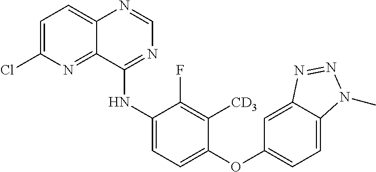 [SMILES: [2H]C([2H])([2H])c1c(Oc2ccc3c(c2)nnn3C)ccc(Nc2ncnc3ccc(Cl)nc23)c1F] | 6-chloro-N-(2-fluoro-3-(methyl-d3)- 4-((1-methyl-1H-benzo[d][1,2,3] triazol-5-yl)oxy)phenyl)pyrido[3,2- d]pyrimidin-4-amine | 439.1 (M + H) |
| I164 | 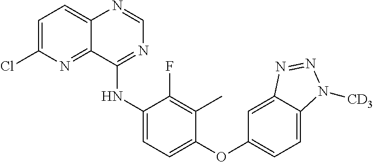 [SMILES: [2H]C([2H])([2H])n1nnc2cc(Oc3ccc(Nc4ncnc5ccc(Cl)nc45)c(F)c3C)ccc21] | 6-chloro-N-(2-fluoro-3-methyl-4- ((1-(methyl-d3)-1H-benzo[d][1,2,3] triazol-5-yl)oxy)phenyl)pyrido[3,2- d]pyrimidin-4-amine | 439.1 (M + H) |

### Example 39 (I53)

### 6-chloro-N-(2,3-difluoro-4-((1-methyl-1H-benzo[d][1,2,3]triazol-5-yl)oxy)phenyl)pyrido[3,2-d]pyrimidin-4-amine

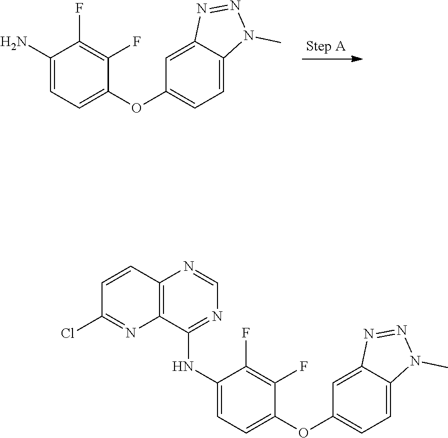 [SMILES: Cn1nnc2cc(Oc3ccc(N)c(F)c3F)ccc21 ; Cn1nnc2cc(Oc3ccc(Nc4ncnc5ccc(Cl)nc45)c(F)c3F)ccc21]

6-chloro-N-(2,3-difluoro-4-((1-methyl-11H-benzo[d][1,2,3]triazol-5-yl)oxy)phenyl)pyrido[3,2-d]pyrimidin-4-amine. To a solution of 2,3-difluoro-4-(1-methylbenzotriazol-5-yl)oxy-aniline (140 mg, 0.51 mmol) and 4,6-dichloropyrido[3,2-d]pyrimidine (101 mg, 0.51 mmol) in n-BuOH (5 mL) was added TFA (38 μL, 0.51 mmol). The mixture was stirred at 100° C. for 1 h and concentrated in vacuo. The residue was diluted with water (5 mL), basified with sat. aq. NaHCO₃ (5 mL) and extracted with EtOAc (20 mL×2). The combined organic layers were dried over anhydrous Na₂SO₄, filtered and concentrated under vacuum. The crude product was purified by silica gel column chromatography (15% EtOAc/PET) to give 6-chloro-N-[2,3-difluoro-4-(1-methylbenzotriazol-5-yl)oxy-phenyl]pyrido[3,2-d]pyrimidin-4-amine (128 mg, 55% yield) as a yellow oil. LCMS (MM-ES+APCI, Pos): m/z 440.0 (M+H).

Intermediates I53, I165-I172 in Table 14 below were prepared according to Example 39 Step A.

TABLE 14
A list of intermediate compounds 153, 1165-1172

| | | | MS |
|---|---|---|---|
| Int. | | | API |
| ID | Structure | Chemical Name | (m/z) |
| I53 | 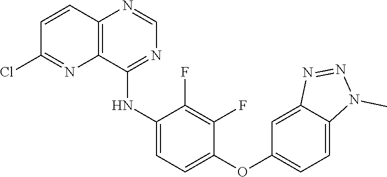 [SMILES: Cn1nnc2cc(Oc3ccc(Nc4ncnc5ccc(Cl)nc45)c(F)c3F)ccc21] | 6-chloro-N-(2,3-difluoro-4-((1-methyl- 1H-benzo[d][1,2,3]triazol-5- yl)oxy)phenyl)pyrido[3,2-d]pyrimidin-4- amine | 440.0 (M + H) |
| I165 | 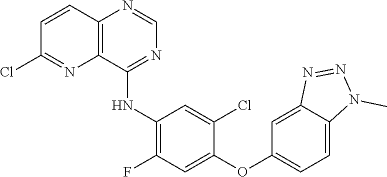 [SMILES: Cn1nnc2cc(Oc3cc(F)c(Nc4ncnc5ccc(Cl)nc45)cc3Cl)ccc21] | 6-chloro-N-(5-chloro-2-fluoro-4-((1- methyl-1H-benzo[d][1,2,3]triazol-5- yl)oxy)phenyl)pyrido[3,2-d]pyrimidin-4- amine | 456.1 (M + H) |
| I166 | 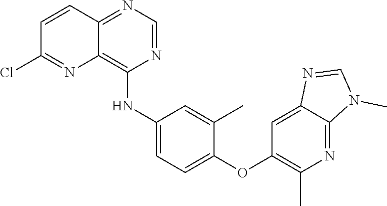 [SMILES: Cc1cc(Nc2ncnc3ccc(Cl)nc23)ccc1Oc1cc2ncn(C)c2nc1C] | 6-chloro-N-(4-((3,5-dimethyl-3H- imidazo[4,5-b]pyridin-6-yl)oxy)-3- methylphenyl)pyrido[3,2-d]pyrimidin-4- amine | 432.1 (M + H) |
| I167 | 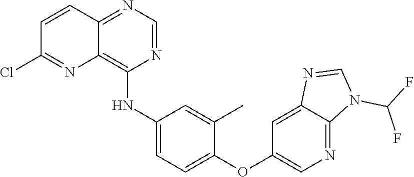 [SMILES: Cc1cc(Nc2ncnc3ccc(Cl)nc23)ccc1Oc1cnc2c(c1)ncn2C(F)F] | 6-chloro-N-(4-((3-(difluoromethyl)-3H- imidazo[4,5-b]pyridin-6-yl)oxy)-3- methylphenyl)pyrido[3,2-d]pyrimidin-4- amine | 454.1 (M + H) |
| I168 | 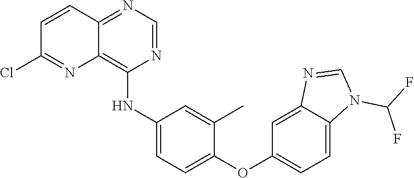 [SMILES: Cc1cc(Nc2ncnc3ccc(Cl)nc23)ccc1Oc1ccc2c(c1)ncn2C(F)F] | 6-chloro-N-(4-((1-(difluoromethyl)-1H- benzo[d]imidazol-5-yl)oxy)-3- methylphenyl)pyrido[3,2-d]pyrimidin-4- amine | 452.9 (M + H) |
| I169 | 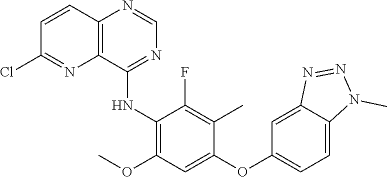 [SMILES: COc1cc(Oc2ccc3c(c2)nnn3C)c(C)c(F)c1Nc1ncnc2ccc(Cl)nc12] | 6-chloro-N-(2-fluoro-6-methoxy-3- methyl-4-((1-methyl-1H- benzo[d][1,2,3]triazol-5- yl)oxy)phenyl)pyrido[3,2-d]pyrimidin-4- amine | 466.1 (M + H) |
| I170 | 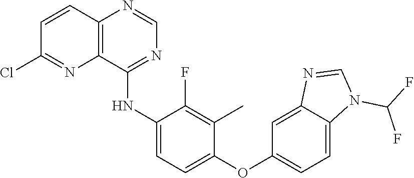 [SMILES: Cc1c(Oc2ccc3c(c2)ncn3C(F)F)ccc(Nc2ncnc3ccc(Cl)nc23)c1F] | 6-chloro-N-(4-((1-(difluoromethyl)-1H- benzo[d]imidazol-5-yl)oxy)-2-fluoro-3- methylphenyl)pyrido[3,2-d]pyrimidin-4- amine | 471.1 (M + H) |
| I171 | 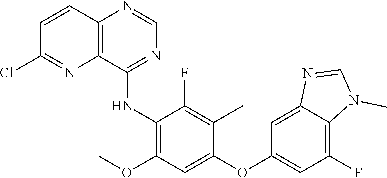 [SMILES: COc1cc(Oc2cc(F)c3c(c2)ncn3C)c(C)c(F)c1Nc1ncnc2ccc(Cl)nc12] | 6-chloro-N-(2-fluoro-4-((7-fluoro-1- methyl-1H-benzo[d]imidazol-5-yl)oxy)- 6-methoxy-3-methylphenyl)pyrido[3,2- d]pyrimidin-4-amine | 483.2 (M + H) |
| I172 | 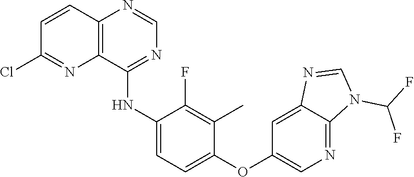 [SMILES: Cc1c(Oc2cnc3c(c2)ncn3C(F)F)ccc(Nc2ncnc3ccc(Cl)nc23)c1F] | 6-chloro-N-(4-((3-(difluoromethyl)-3H- imidazo[4,5-b]pyridin-6-yl)oxy)-2- fluoro-3-methylphenyl)pyrido[3,2- d]pyrimidin-4-amine | 472.2 (M + H) |

### Example 40 (I173)

### 6-fluoro-N-(2-fluoro-3-methyl-4-((1-methyl-H-benzo[d][1,2,3]triazol-5-yl)oxy)phenyl)pyrido[3,2-d]pyrimidin-4-amine

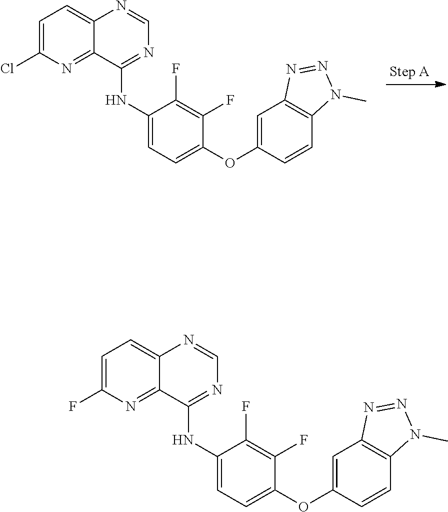 [SMILES: Cn1nnc2cc(Oc3ccc(Nc4ncnc5ccc(Cl)nc45)c(F)c3F)ccc21 ; Cn1nnc2cc(Oc3ccc(Nc4ncnc5ccc(F)nc45)c(F)c3F)ccc21]

Step A. 6-fluoro-N-(2-fluoro-3-methyl-4-((1-methyl-1H-benzo[d][1,2,3]triazol-5-yl)oxy)phenyl)pyrido[3,2-d]pyrimidin-4-amine. To a solution of 6-chloro-N-[2-fluoro-3-methyl-4-(1-methylbenzotriazol-5-yl)oxy-phenyl]pyrido[3,2-d]pyrimidin-4-amine (140 mg, 0.32 mmol) in DMSO (6 mL) were added KF (93.3 mg, 1.61 mmol) and DIPEA (0.28 mL, 1.61 mmol). The reaction was stirred at 100° C. for 4 h. The mixture was concentrated under reduced pressure to afford 6-fluoro-N-[2-fluoro-3-methyl-4-(1-methylbenzotriazol-5-yl)oxy-phenyl]pyrido[3,2-d]pyramid in-4-amine 7 (200 mg, quant. yield) as a light yellow oil. LCMS (MM-ES+APCI, Pos): m/z 420.1 (M+H).

### Example 41 (I54)

### tert-butyl 4-(4-chloropyrido[3,2-d]pyrimidin-6-yl)piperazine-1-carboxylate

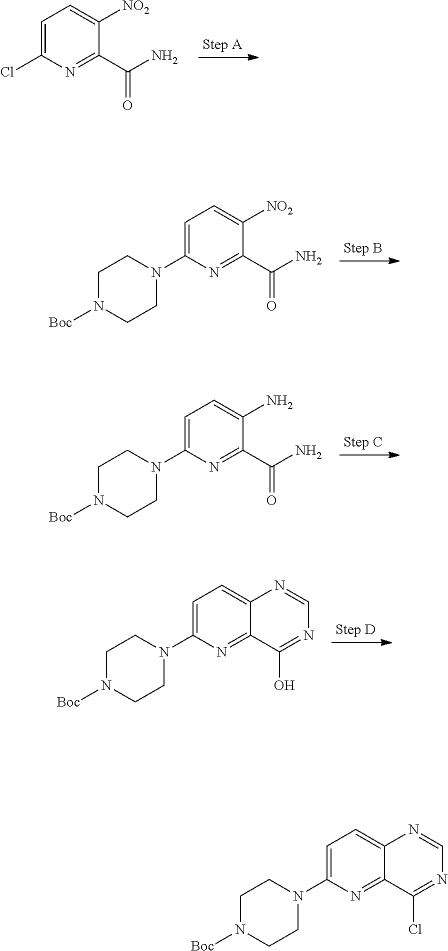 [SMILES: NC(=O)c1nc(Cl)ccc1[N+](=O)[O-] ; CC(C)(C)OC(=O)N1CCN(c2ccc([N+](=O)[O-])c(C(N)=O)n2)CC1 ; CC(C)(C)OC(=O)N1CCN(c2ccc(N)c(C(N)=O)n2)CC1 ; CC(C)(C)OC(=O)N1CCN(c2ccc3ncnc(O)c3n2)CC1 ; CC(C)(C)OC(=O)N1CCN(c2ccc3ncnc(Cl)c3n2)CC1]

Step A. tert-butyl 4-(6-carbamoyl-5-nitropyridin-2-yl)piperazine-1-carboxylate. To a solution of 6-chloro-3-nitro-pyridine-2-carboxamide (1.00 g, 4.96 mmol) in DMSO (30 mL) was added DIPEA (2.59 mL, 14.9 mmol) and tert-butyl piperazine-1-carboxylate (1.39 g, 7.44 mmol). The mixture was stirred at 90° C. for 1 h, quenched with H₂O (100 mL), and extracted with EtOAc (100 mL×3). The combined organic layers were washed with brine (100 mL), dried over anhydrous Na₂SO₄, filtered, and concentrated under reduced pressure to afford tert-butyl 4-(6-carbamoyl-5-nitro-2-pyridyl)piperazine-1-carboxylate (2.0 g, quant. yield) as a white solid. LCMS (MM-ES+APCI, Pos): m/z 352.0 (M+H).

Step B. tert-butyl 4-(5-amino-6-carbamoylpyridin-2-yl)piperazine-1-carboxylate. To a solution of tert-butyl 4-(6-carbamoyl-5-nitro-2-pyridyl)piperazine-1-carboxylate (1.8 g, 5.12 mmol) in EtOH (20 mL) and H₂O (10 mL) was added Fe powder (1.14 g, 20.5 mmol) and NH₄Cl (1.10 g, 20.5 mmol). The mixture was stirred at 80° C. for 1 h. The suspension was filtered and the filter cake was washed with MeOH (20 mL). The filtrate was concentrated in vacuo to provide tert-butyl 4-(5-amino-6-carbamoyl-2-pyridyl)piperazine-1-carboxylate 4 (1.8 g, quant.) as a gray solid. LCMS (MM-ES+APCI, Pos): m/z 322.1 (M+H).

Step C. tert-butyl 4-(8-hydroxypyrimido[5,4-d]pyrimidin-2-yl)piperazine-1-carboxylate. A solution of tert-butyl 4-(5-amino-6-carbamoyl-2-pyridyl)piperazine-1-carboxylate (1.7 g, 5.29 mmol) in CH(OEt)₃ (20 mL) was heated to 100° C. for 2 h. The reaction was quenched with water (50 mL) and extracted with EtOAc (50 mL×3). The combined organic layers were washed with brine (50 mL), dried over anhydrous Na₂SO₄, filtered, and concentrated under reduced pressure. The crude material was purified by silica gel column chromatography (25% to 35% EtOAc/PET) to provide tert-butyl 4-(4-hydroxypyrido[3,2-d]pyrimidin-6-yl)piperazine-1-carboxylate (1.1 g, 60% yield) as a brown solid. LCMS (MM-ES+APCI, Pos): m/z 332.1 (M+H).

Step D. tert-butyl 4-(8-chloropyrimido[5,4-d]pyrimidin-2-yl)piperazine-1-carboxylate. A solution of tert-butyl 4-(4-hydroxypyrido[3,2-d]pyrimidin-6-yl)piperazine-1-carboxylate (527 mg, 1.51 mmol) in toluene (10 mL) was cooled to 0° C., followed by addition of POCl₃ (0.18 mL, 1.96 mmol) and DIPEA (1.31 mL, 7.54 mmol). The mixture was stirred at 80° C. for 2 h and concentrated in vacuo to afford tert-butyl 4-(4-chloropyrido[3,2-d]pyrimidin-6-yl)piperazine-1-carboxylate (800 mg, quant.) as a brown oil. LCMS (MM-ES+APCI, Pos): m/z 350.0 (M+H).

### Example 42 (I55)

### tert-butyl 3-(4-chloropyrido[3,2-d]pyrimidin-6-yl)-3,6-diazabicyclo[3.1.1]heptane-6-carboxylate

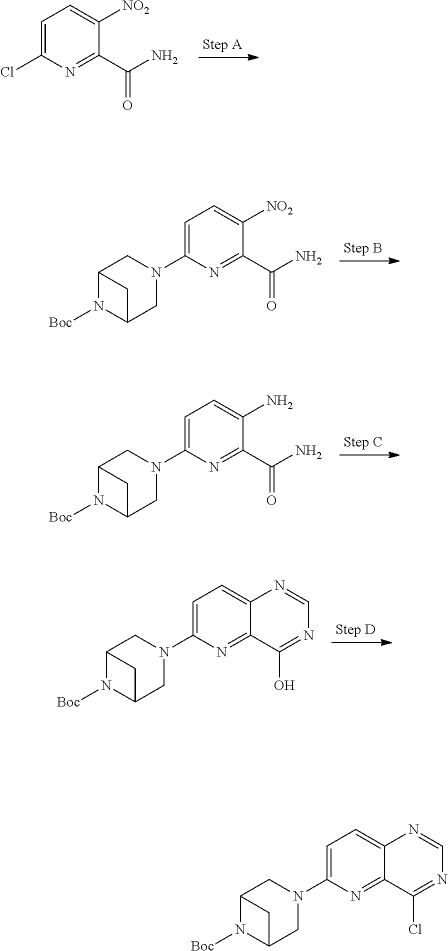 [SMILES: NC(=O)c1nc(Cl)ccc1[N+](=O)[O-] ; CC(C)(C)OC(=O)N1C2CC1CN(c1ccc([N+](=O)[O-])c(C(N)=O)n1)C2 ; CC(C)(C)OC(=O)N1C2CC1CN(c1ccc(N)c(C(N)=O)n1)C2 ; CC(C)(C)OC(=O)N1C2CC1CN(c1ccc3ncnc(O)c3n1)C2 ; CC(C)(C)OC(=O)N1C2CC1CN(c1ccc3ncnc(Cl)c3n1)C2]

tert-butyl 3-(4-chloropyrido[3,2-d]pyrimidin-6-yl)-3,6-diazabicyclo[3.1.1]heptane-6-carboxylate. Prepared according to Example 41 Steps A to D, substituting tert-butyl 3,6-diazabicyclo[3.1.1]heptane-6-carboxylate for tert-butyl piperazine-1-carboxylate to provide tert-butyl 3-(4-chloropyrido[3,2-d]pyrimidin-6-yl)-3,6-diazabicyclo[3.1.1]heptane-6-carboxylate (350 mg, quant. yield) as a black oil. LCMS (MM-ES+APCI, Pos): m/z 362.0 (M+H).

### Example 43 (I56)

### tert-butyl 6-(4-chloropyrido[3,2-d]pyrimidin-6-yl)-3,6-diazabicyclo[3.1.1]heptane-3-carboxylate

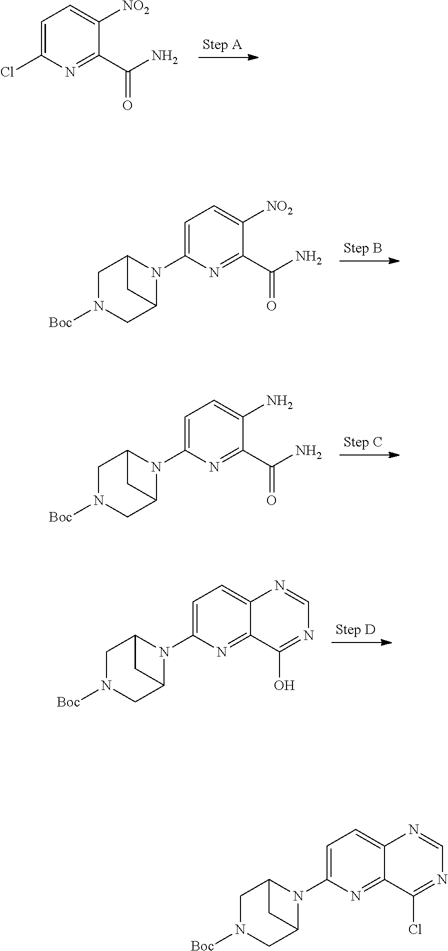 [SMILES: NC(=O)c1nc(Cl)ccc1[N+](=O)[O-] ; CC(C)(C)OC(=O)N1CC2CC(C1)N2c1ccc([N+](=O)[O-])c(C(N)=O)n1 ; CC(C)(C)OC(=O)N1CC2CC(C1)N2c1ccc(N)c(C(N)=O)n1 ; CC(C)(C)OC(=O)N1CC2CC(C1)N2c1ccc2ncnc(O)c2n1 ; CC(C)(C)OC(=O)N1CC2CC(C1)N2c1ccc2ncnc(Cl)c2n1]

tert-butyl 6-(4-chloropyrido[3,2-d]pyrimidin-6-yl)-3,6-diazabicyclo[3.1.1]heptane-3-carboxylate. Prepared according to Example 41 Steps A to D, substituting tert-butyl 3,6-diazabicyclo[3.1.1]heptane-3-carboxylate for tert-butyl piperazine-1-carboxylate to provide tert-butyl 6-(4-chloropyrido[3,2-d]pyrimidin-6-yl)-3,6-diazabicyclo[3.1.1]heptane-3-carboxylate (420 mg, quant. yield) as a brown oil. LCMS (MM-ES+APCI, Pos): m/z 362.0 (M+H).

### Example 44 (I57)

### tert-butyl 4-(4-chloro-5-fluoropyrido[3,4-d]pyrimidin-6-yl)piperazine-1-carboxylate

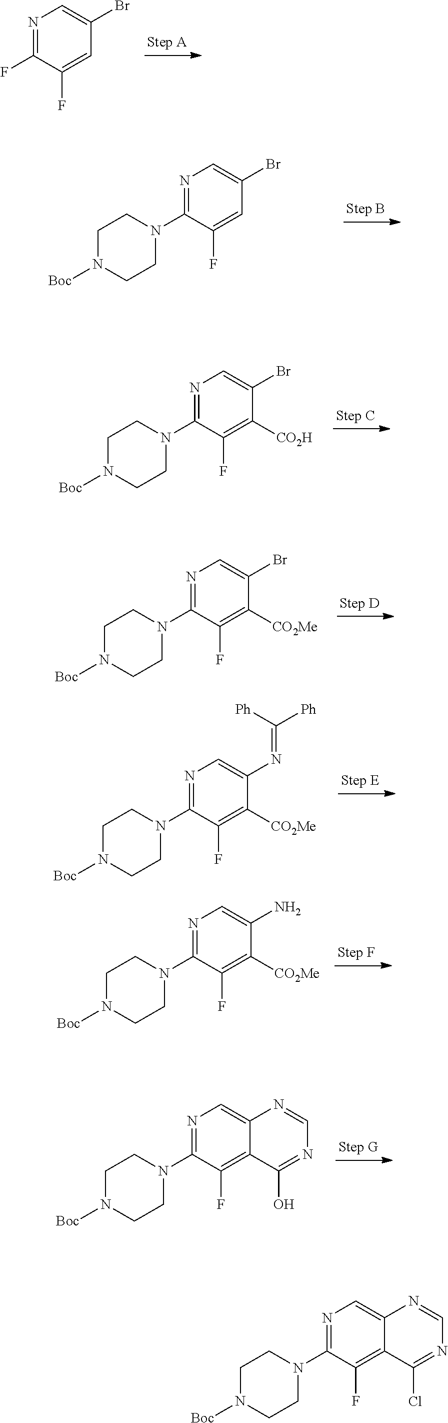

Step A. tert-butyl 4-(5-bromo-3-fluoropyridin-2-yl)piperazine-1-carboxylate. To a solution of 5-bromo-2,3-difluoro-pyridine (10.0 g, 51.6 mmol) in DMF (100 mL) were added K₂CO₃ (9.26 g, 67.0 mmol) and tert-butyl piperazine-1-carboxylate (9.60 g, 51.6 mmol). The reaction was stirred at 100° C. for 2 h, quenched slowly with water (100 mL), and extracted with EtOAc (100 mL×3). The combined organic layers were washed with water (100 mL×2) and brine (100 mL), dried over anhydrous Na₂SO₄, filtered and concentrated in vacuo. The crude oil was purified by silica gel column chromatography (1% to 30% EtOAc/PET) to give tert-butyl 4-(5-bromo-3-fluoro-2-pyridyl) piperazine-1-carboxylate (11 g, 59% yield) as a white solid. LCMS (MM-ES+APCI, Pos): m/z 304.1 (M+H-^{t}Bu).

Step B. 5-bromo-2-(4-(tert-butoxycarbonyl)piperazin-1-yl)-3-fluoroisonicotinic acid. A solution of tert-butyl 4-(5-bromo-3-fluoro-2-pyridyl)piperazine-1-carboxylate (10.0 g, 27.8 mmol) in THF (30 mL) was cooled to −78° C. LDA (2M in THF, 20.8 mL, 41.6 mmol) was added dropwise, and the reaction was stirred for 30 min. Solid CO₂ (1.22 g, 27.8 mmol) was added, and stirring continued at −78° C. for 2 h. The mixture was quenched slowly with sat. aq. NH₄Cl (50 mL), warmed to 25° C., and extracted with EtOAc (50 mL×3). The combined organic layers were washed with brine (30 mL), dried over anhydrous Na₂SO₄, filtered and concentrated in vacuo. The crude oil was purified by silica gel column chromatography (0% to 90% EtOAc/PET) to afford 5-bromo-2-(4-tert-butoxycarbonylpiperazin-1-yl)-3-fluoro-pyridine-4-carboxylic acid (8.0 g, 71% yield) as a yellow oil. LCMS (MM-ES+APCI, Pos): m/z 348.0 (M+H-^{t}Bu).

Step C. tert-butyl 4-(5-bromo-3-fluoro-4-(methoxycarbonyl)pyridin-2-yl)piperazine-1-carboxylate. To a solution of 5-bromo-2-(4-tert-butoxycarbonylpiperazin-1-yl)-3-fluoro-pyridine-4-carboxylic acid (8.0 g, 19.8 mmol) in DMF (80 mL) were added K₂CO₃ (5.47 g, 39.6 mmol) and Mel (6.16 mL, 99.0 mmol). The mixture was stirred at 20° C. for 2 h, quenched with water (30 mL), and extracted with EtOAc (40 mL×3). The combined organic layers were washed with water (100 mL×2) and brine (100 mL), dried over anhydrous Na₂SO₄, filtered and concentrated under reduced pressure. The crude oil was purified by silica gel column chromatography (1% to 20% EtOAc/PET) to give tert-butyl 4-(5-bromo-3-fluoro-4-methoxycarbonyl-2-pyridyl) piperazine-1-carboxylate (6.0 g, 72% yield) as a yellow oil. LCMS (MM-ES+APCI, Pos): m/z 364.1 (M+H-^{t}Bu).

Step D. tert-butyl 4-(5-((diphenylmethylene)amino)-3-fluoro-4-(methoxycarbonyl)pyridin-2-yl)piperazine-1-carboxylate. A solution of tert-butyl 4-(5-bromo-3-fluoro-4-methoxycarbonyl-2-pyridyl)piperazine-1-carboxylate (5.0 g, 12.0 mmol) in 1,4-dioxane (50 mL) was purged with N₂. Pd₂(dba)₃ (1.09 g, 1.20 mmol), Xantphos (1.04 g, 1.79 mmol), Cs₂CO₃ (9.74 g, 30.0 mmol) and diphenylmethanimine (2.41 mL, 14.4 mmol) were added and the mixture was heated to 100° C. for 2 h. The reaction was quenched with water (20 mL) and extracted with EtOAc (30 mL×3). The combined organic layers were washed with brine (30 mL), dried over anhydrous Na₂SO₄, filtered and concentrated under reduced pressure. The crude solid was purified by silica gel column chromatography (1% to 50% EtOAc/PET) to afford tert-butyl 4-[5-(benzhydrylideneamino)-3-fluoro-4-methoxycarbonyl-2-pyridyl]piperazine-1-carboxylate (5.8 g, 94% yield) as a yellow solid. LCMS (MM-ES+APCI, Pos): m/z 519.5 (M+H).

Step E. tert-butyl 4-(5-amino-3-fluoro-4-(methoxycarbonyl)pyridin-2-yl)piperazine-1-carboxylate. To a solution of tert-butyl 4-[5-(benzhydrylideneamino)-3-fluoro-4-methoxycarbonyl-2-pyridyl]piperazine-1-carboxylate (5.8 g, 11.2 mmol) in MeOH (20 mL) and water (20 mL) was added 2M HCl (11.2 mL, 22.4 mmol). The mixture was stirred at 20° C. for 2 h, neutralized (pH 7) with sat. aq. NaHCO₃ (200 mL) and extracted with EtOAc (60 mL×3). The combined organic layers were washed with brine (60 mL), dried over Na₂SO₄, filtered, and concentrated in vacuo. The crude residue was purified was purified by silica gel column chromatography (1% to 50% EtOAc/PET) to afford tert-butyl 4-(5-amino-3-fluoro-4-methoxycarbonyl-2-pyridyl) piperazine-1-carboxylate (3.1 g, 78% yield) as a yellow oil. LCMS (MM-ES+APCI, Pos): m/z 355.2 (M+H).

Step F. tert-butyl 4-(5-fluoro-4-hydroxypyrido[3,4-d]pyrimidin-6-yl)piperazine-1-carboxylate. To a solution of tert-butyl 4-(5-amino-3-fluoro-4-methoxycarbonyl-2-pyridyl)piperazine-1-carboxylate (3.0 g, 8.47 mmol) in 2-methoxyethanol (30 mL) were added NaOAc (2.08 g, 25.4 mmol) and formamidine hydrochloride (2.73 g, 33.9 mmol). The mixture was stirred for 2 h at 110° C., quenched with water (30 mL), and extracted with EtOAc (40 mL×3). The combined organic layers were washed with brine (30 mL), dried over anhydrous Na₂SO₄, filtered, and concentrated under reduced pressure. The crude oil was purified by silica gel column chromatography (1% to 50% EtOAc/PET) to provide tert-butyl 4-(5-fluoro-4-hydroxy-pyrido [3,4-d]pyrimidin-6-yl) piperazine-1-carboxylate (1.9 g, 64% yield) as a yellow solid. LCMS (MM-ES+APCI, Pos): m/z 294.0 (M+H-^{t}Bu).

Step G. tert-butyl 4-(4-chloro-5-fluoropyrido[3,4-d]pyrimidin-6-yl)piperazine-1-carboxylate. To a solution of tert-butyl 4-(5-fluoro-4-hydroxy-pyrido[3,4-d]pyrimidin-6-yl)piperazine-1-carboxylate (1.8 g, 5.15 mmol) in PhMe (20 mL) were added DIPEA (4.5 mL, 25.8 mmol) and POCl₃ (0.58 mL, 6.18 mmol). The mixture was stirred for 2 h at 100° C., quenched slowly with sat. aq. NaHCO₃ (30 mL) and extracted with EtOAc (30 mL×3). The combined organic layers were washed with brine (30 mL), dried over anhydrous Na₂SO₄, filtered and concentrated under reduced pressure. The crude solid was purified by silica gel column chromatography (1% to 50% EtOAc/PET) to afford tert-butyl 4-(4-chloro-5-fluoro-pyrido [3,4-d]pyrimidin-6-yl) piperazine-1-carboxylate (1.10 g, 58% yield) as a yellow solid. LCMS (MM-ES+APCI, Pos): m/z 368.2 (M+H).

### Example 45 (I58)

### tert-butyl 4-(4-chloro-7-methoxypyrido[3,2-d]pyrimidin-6-yl)piperazine-1-carboxylate

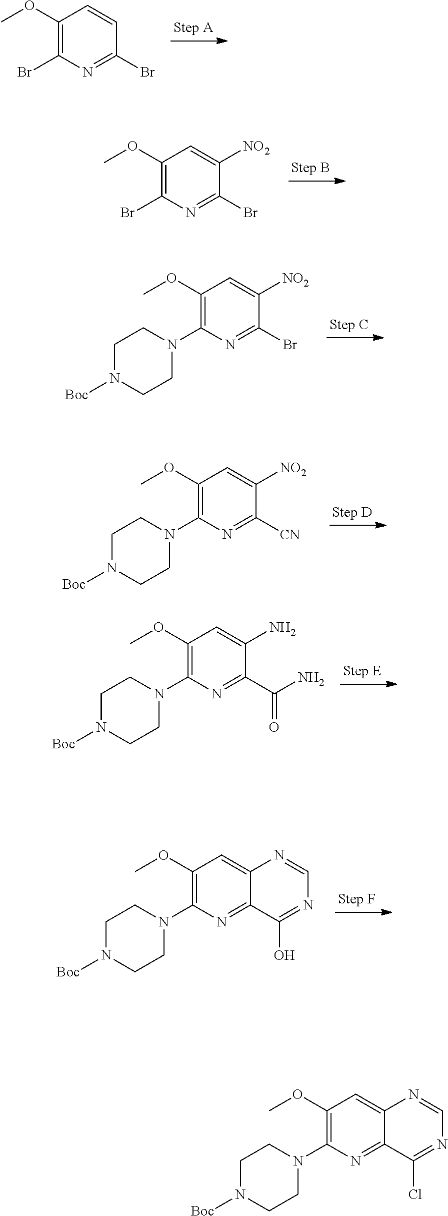

Step A. 2,6-dibromo-3-methoxy-5-nitropyridine. Nitric acid (32.9 mL, 732 mmol) was added dropwise to ice cold sulfuric acid (30 mL), followed by portion wise addition of 2,6-dibromo-3-methoxy-pyridine (7.50 g, 28.1 mmol). The mixture was stirred for 20 min at 0° C., 10 min at 10° C., and 2.5 h at 60° C. The reaction was slowly poured into ice water (200 mL), and the yellow precipitate was collected, washed with water (800 mL), and dried in vacuo to afford 2,6-dibromo-3-methoxy-5-nitro-pyridine (3.32 g, 36% yield) as a light-yellow solid. LCMS (MM-ES+APCI, Pos): m/z 312.6 (M+H).

Step B. tert-butyl 4-(6-bromo-3-methoxy-5-nitropyridin-2-yl)piperazine-1-carboxylate. To a solution of 2,6-dibromo-3-methoxy-5-nitro-pyridine (1.00 g, 3.21 mmol) in DMF (20 mL) were added K₂CO₃ (1.33 g, 9.62 mmol) and tert-butyl piperazine-1-carboxylate (597 mg, 3.21 mmol). The mixture was stirred at 25° C. for 1 h, diluted with water (50 mL), and extracted with EtOAc (40 mL×2). The combined organic layers were washed with brine (40 mL), dried over anhydrous Na₂SO₄, filtered and concentrated in vacuo to afford tert-butyl 4-(6-bromo-3-methoxy-5-nitro-2-pyridyl)piperazine-1-carboxylate (1.70 g, 88% yield) as a yellow solid. LCMS (MM-ES+APCI, Pos): m/z 439.0 (M+Na⁺).

Step C. tert-butyl 4-(6-cyano-3-methoxy-5-nitropyridin-2-yl)piperazine-1-carboxylate. To a solution of tert-butyl 4-(6-bromo-3-methoxy-5-nitro-2-pyridyl)piperazine-1-carboxylate (1.70 g, 4.07 mmol) in DMF (40 mL) was added CuCN (438 mg, 4.89 mmol). The mixture was stirred at 100° C. for 1 h, diluted with water (60 mL), and extracted with EtOAc (50 mL×2). The combined organic layers were washed with brine (60 mL×2), dried over anhydrous Na₂SO₄, filtered and concentrated in vacuo to afford tert-butyl 4-(6-cyano-3-methoxy-5-nitro-2-pyridyl)piperazine-1-carboxylate (1.22 g, 68% yield) as a yellow solid. LCMS (MM-ES+APCI, Pos): m/z 386.0 (M+Na⁺).

Step D. tert-butyl 4-(5-amino-6-carbamoyl-3-methoxypyridin-2-yl)piperazine-1-carboxylate. To a solution of tert-butyl 4-(6-cyano-3-methoxy-5-nitro-2-pyridyl)piperazine-1-carboxylate (1.22 g, 3.36 mmol) in EtOH (30 mL) and water (10 mL) were added Fe powder (937 mg, 16.8 mmol) and NH₄Cl (1.80 g, 33.6 mmol). The mixture was stirred at 80° C. for 1 h and filtered. The filtrate was concentrated in vacuo, diluted with water (40 mL), and extracted with EtOAc (30 mL×3). The combined organic layers were dried over anhydrous Na₂SO₄, filtered, and concentrated under reduced pressure to afford tert-butyl 4-(5-amino-6-carbamoyl-3-methoxy-2-pyridyl)piperazine-1-carboxylate (1.14 g, 73% yield) as a brown solid. LCMS (MM-ES+APCI, Pos): m/z 352.1 (M+H).

Step E. tert-butyl 4-(4-hydroxy-7-methoxypyrido[3,2-d]pyrimidin-6-yl)piperazine-1-carboxylate. A solution of tert-butyl 4-(5-amino-6-carbamoyl-3-methoxy-2-pyridyl)piperazine-1-carboxylate (1.14 g, 3.24 mmol) in CH(OEt)₃ (15 mL) was heated to 100° C. and stirred for 2 h. The mixture was concentrated in vacuo and the residue purified by silica gel column chromatography (5% to 10% MeOH/DCM) to provide tert-butyl 4-(4-hydroxy-7-methoxy-pyrido[3,2-d]pyrimidin-6-yl)piperazine-1-carboxylate (350 mg, 27% yield) as a brown solid. LCMS (MM-ES+APCI, Pos): m/z 362.3 (M+H).

Step F. tert-butyl 4-(4-chloro-7-methoxypyrido[3,2-d]pyrimidin-6-yl)piperazine-1-carboxylate hydrochloride. To a solution of tert-butyl 4-(4-hydroxy-7-methoxy-pyrido[3,2-d]pyrimidin-6-yl)piperazine-1-carboxylate (145 mg, 0.40 mmol) in toluene (1 mL) were added DIPEA (0.28 mL, 1.60 mmol) and POCl₃ (0.37 mL, 4.01 mmol). The reaction mixture was stirred at 90° C. for 1 h and concentrated in vacuo to afford tert-butyl 4-(4-chloro-7-methoxy-pyrido[3,2-d]pyrimidin-6-yl)piperazine-1-carboxylate hydrochloride (167 mg, quant. yield) as a brown solid. LCMS (MM-ES+APCI, Pos): m/z 380.3 (M+H—HCl).

### Example 46 (I59)

### 1-(4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one

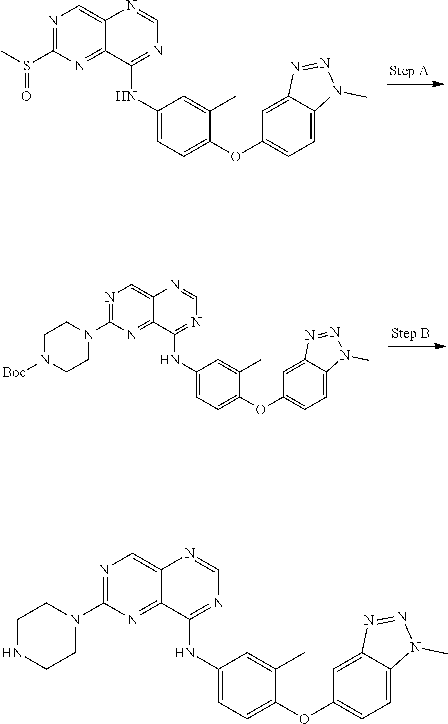

Step A. tert-butyl 4-(8-((3-methyl-4-((1-methyl-1H-benzo[d][1,2,3]triazol-5-yl)oxy)phenyl)amino)pyrimido[5,4-d]pyrimidin-2-yl)piperazine-1-carboxylate. To a solution of N-(3-methyl-4-((1-methyl-1H-benzo[d][1,2,3]triazol-5-yl)oxy)phenyl)-6-(methylsulfinyl)pyrimido[5,4-d]pyrimidin-4-amine (5.10 g, 11.4 mmol) and tert-butyl piperazine-1-carboxylate (4.26 g, 22.8 mmol) in 1,4-dioxane (62 mL) was added DIPEA (3.98 mL, 22.8 mmol). The reaction was heated to 80° C. for 2 h and concentrated in vacuo. The solids were purified using silica gel column chromatography (0% to 100% EtOAc/DCM) to give tert-butyl 4-(8-((3-methyl-4-((1-methyl-1H-benzo[d][1,2,3]triazol-5-yl)oxy)phenyl)amino)pyrimido[5,4-d]pyrimidin-2-yl)piperazine-1-carboxylate (6.41 g, 99% yield). LCMS (MM-ES+APCI, Pos): m/z 569.3 (M+H).

Step B. N-(3-methyl-4-((1-methyl-1H-benzo[d][1,2,3]triazol-5-yl)oxy)phenyl)-6-(piperazin-1-yl)pyrimido[5,4-d]pyrimidin-4-amine. To a solution of tert-butyl 4-(8-((3-methyl-4-((1-methyl-1H-benzo[d]1[1,2,3]triazol-5-yl)oxy)phenyl)amino)pyrimido[5,4-d]pyrimidin-2-yl)piperazine-1-carboxylate (48.1 g, 84.6 mmol) in DCM (423 mL) was added TFA (65.2 mL, 846 mmol). The reaction was stirred at ambient temperature for 15 h and concentrated in vacuo. The mixture was dissolved in 20% MeOH/DCM and neutralized (pH 7) with 1M NaOH. The layers were separated and the organics were washed with sat. aq. NaHCO₃ (2×), water, and brine. The organics were dried over Na₂SO₄, filtered, and concentrated in vacuo. The crude material was purified using silica gel column chromatography (0% to 20% MeOH/DCM) to give N-(3-methyl-4-((1-methyl-H-benzo[d][1,2,3]triazol-5-yl)oxy)phenyl)-6-(piperazin-1-yl)pyrimido[5,4-d]pyrimidin-4-amine (35.2 g, 89% yield). LCMS (MM-ES+APCI, Pos): m/z 469.3 (M+H).

Alternative Step B. N-(3-methyl-4-((1-methyl-H-benzo[d][1,2,3]triazol-5-yl)oxy)phenyl)-6-(piperazin-1-yl)pyrimido[5,4-d]pyrimidin-4-amine hydrochloride. To an ice cold solution of tert-butyl 4-[4-[3-methyl-4-(1-methylbenzotriazol-5-yl)oxy-anilino]pyrimido[5,4-d]pyrimidin-6-yl]piperazine-1-carboxylate (410 mg, 0.72 mmol) in DCM (10 mL) was added HCl (4M in EtOAc, 4 mL, 16 mmol). The mixture was stirred at 0° C. for 1 h and concentrated in vacuo to provide N-[3-methyl-4-(1-methylbenzotriazol-5-yl)oxy-phenyl]-6-piperazin-1-yl-pyrimido[5,4-d]pyrimidin-4-amine hydrochloride (435 mg, quant. yield) as a yellow solid. LCMS (MM-ES+APCI, Pos): m/z 469.1 (M+H).

### Example 47 (I60)

### (E)-4-(3-fluoroazetidin-1-yl)but-2-enoic acid

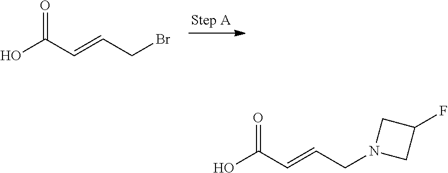 [SMILES: O=C(O)/C=C/CBr ; O=C(O)/C=C/CN1CC(F)C1]

Step A. (E)-4-(3-fluoroazetidin-1-yl)but-2-enoic acid. To a solution of 3-fluoroazetidine hydrochloride (500 mg, 4.48 mmol) and (E)-4-bromobut-2-enoic acid (740 mg, 4.48 mmol) in DMF (10 mL) was added DIPEA (3.12 mL, 7.93 mmol) dropwise at 25° C. The mixture was heated to 60° C. and stirred for 1 h and concentrated in vacuo to afford (E)-4-(3-fluoroazetidin-1-yl)but-2-enoic acid (945 mg, quant.) as yellow oil. LCMS (MM-ES+APCI, Neg): m/z 158.2 (M−H⁺).

### Example 48 (I61)

### 1-(4-hydroxypiperidin-1-yl)prop-2-en-1-one

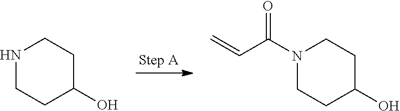

Step A. 1-(4-hydroxypiperidin-1-yl)prop-2-en-1-one. To a solution of piperidin-4-ol (10.0 g, 98.9 mmol) in DCM (15 mL) was added TEA (27.5 mL, 198 mmol) and acryloyl chloride (8.1 mL, 98.9 mmol) at 0° C. The reaction was stirred under N₂ for 2 h at 0° C. and for 12 h at 25° C. The mixture was concentrated under reduced pressure and the crude material was purified by preparative HPLC (20% MeCN/0.05% aqueous NH₄OH) to afford 1-(4-hydroxypiperidin-1-yl)prop-2-en-1-one (5.5 g, 32% yield) as a yellow oil. LCMS (MM-ES+APCI, Pos): m/z 156.2 (M+H).

### Example 49 (I174)

### 1-(3-hydroxypyrrolidin-1-yl)prop-2-en-1-one

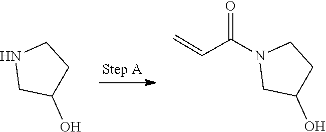

Step A. 1-(3-hydroxypyrrolidin-1-yl)prop-2-en-1-one. Prepared according to example 48 substituting pyrrolidin-3-ol for piperidin-4-ol to afford 1-(3-hydroxypyrrolidin-1-yl)prop-2-en-1-one (1.02 g, 52% yield) as a white solid. LCMS (MM-ES+APCI, Pos): m/z 283.2 (2×M+H).

### Example 50 (I62)

### 1-(3-(methylamino)azetidin-1-yl)prop-2-en-1-one 2,2,2-trifluoroacetate

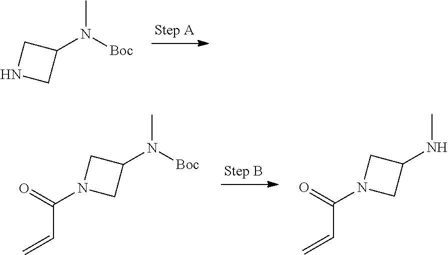

Step A. tert-butyl (1-acryloylazetidin-3-yl)(methyl)carbamate. To a solution of tert-butyl N-(azetidin-3-yl)-N-methyl-carbamate hydrochloride (300 mg, 1.35 mmol) in THF (5 mL) was added sat. aq. NaHCO₃ (0.15 mL, 4.04 mmol). After addition, the mixture was cooled to 0° C. and prop-2-enoyl chloride (0.22 mL, 2.69 mmol) was added dropwise. The reaction was stirred at 20° C. for 0.5 h and quenched slowly with sat. aq. NaHCO₃ (10 mL). The mixture was extracted with DCM (15 mL×3), and the combined organic layers were washed with brine (15 mL), dried over anhydrous Na₂SO₄, filtered, and concentrated under reduced pressure to afford tert-butyl N-methyl-N-(1-prop-2-enoylazetidin-3-yl)carbamate (300 mg, 93% yield) as a light yellow oil.

Step B. 1-(3-(methylamino)azetidin-1-yl)prop-2-en-1-one 2,2,2-trifluoroacetate. To a solution of tert-butyl N-methyl-N-(1-prop-2-enoylazetidin-3-yl)carbamate (300 mg, 1.25 mmol) in DCM (3 mL) was added TFA (1.00 mL, 13.5 mmol). The mixture was stirred at 20° C. for 0.5 h and concentrated in vacuo to provide 1-[3-(methylamino)azetidin-1-yl]prop-2-en-I-one 2,2,2-trifluoroacetate (300 mg, 1.18 mmol, 95% yield) as a light yellow oil. LCMS (MM-ES+APCI, Pos): m/z 141.0 (M+H-TFA).

### Example 51 (I63)

### (E)-4-(pyrrolidin-1-yl)but-2-enoic acid

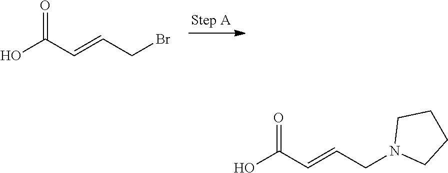 [SMILES: O=C(O)/C=C/CBr ; O=C(O)/C=C/CN1CCCC1]

Step A. (E)-4-(pyrrolidin-1-yl)but-2-enoic acid. To a solution of pyrrolidine (0.33 mL, 3.97 mmol) and (E)-4-bromoprop-2-enoic acid (400 mg, 2.65 mmol) in DMF (10 mL) was added DIPEA (1.38 mL, 7.95 mmol). The mixture was heated to 60° C., stirred for 1 h, and concentrated in vacuo to afford (E)-4-pyrrolidin-1-ylbut-2-enoic acid (1.4 g, quant. yield) as a yellow oil. LCMS (MM-ES+APCI, Neg): m/z 154.2 (M−H⁺).

### Example 52 (I64)

### (E)-4-hydroxybut-2-enoic acid

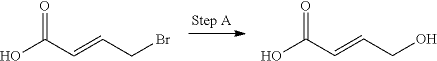 [SMILES: O=C(O)/C=C/CBr ; O=C(O)/C=C/CO]

Step A. (E)-4-hydroxybut-2-enoic acid. To a solution of KOH (408 mg, 7.27 mmol) in H₂O (4 mL) was added (E)-4-bromobut-2-enoic acid (300 mg, 1.82 mmol). The mixture was heated to 60° C., stirred for 1 h, and concentrated in vacuo to afford (E)-4-hydroxybut-2-enoic acid (510 mg, quant. yield) as a white solid. LCMS (MM-ES+APCI, Neg): m/z 101.1 (M−H⁺).

### Example 53 (I175)

### (E)-4-fluorobut-2-enoic acid

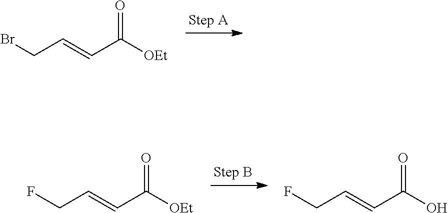 [SMILES: CCOC(=O)/C=C/CBr ; CCOC(=O)/C=C/CF ; O=C(O)/C=C/CF]

Step A. ethyl (E)-4-fluorobut-2-enoate. To a solution of AgF (986 mg, 7.77 mmol) followed in MeCN (10 mL) was added a solution of ethyl (E)-4-bromobut-2-enoate (0.36 mL, 2.59 mmol) in MeCN (6 mL). The vial was purged with N₂ and the reaction was stirred in the dark at 25° C. for 24 h. The mixture was filtered and the filter cake was washed with DCM (20 mL). The filtrate was concentrated in vacuo to provide ethyl (E)-4-fluorobut-2-enoate 7 (340 mg, 99% yield) as a brown oil.

Step B. (E)-4-fluorobut-2-enoic acid. To a solution of ethyl (E)-4-fluorobut-2-enoate 7 (300 mg, 2.27 mmol) in THF (3 mL) and water (3 mL) was added LiOH·H₂O (286 mg, 6.81 mmol). The reaction was stirred at 25° C. for 2.5 h. The mixture was acidified with 1M HCl (15 mL) to pH=4. The aqueous mixture was extracted with DCM (20 mL×3). The combined organic phases were washed with brine (20 mL), dried over sodium sulfate and concentrated in vacuo to give (E)-4-fluorobut-2-enoic acid 5 (60 mg, 25% yield) as a yellow oil. (MM-ES+APCI, Neg): m/z 103.0 (M−H⁺).

### Example 54 (I176)

### tert-butyl (2S,3S)-2-methyl-3-(methylamino)pyrrolidine-1-carboxylate

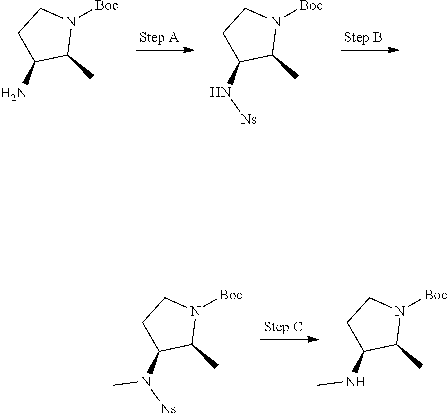

Step A. tert-butyl (2S,3S)-2-methyl-3-((4-nitrophenyl)sulfonamido)pyrrolidine-1-carboxylate. To a solution of tert-butyl (2S,3S)-3-amino-2-methyl-pyrrolidine-1-carboxylate (430 mg, 2.15 mmol) in THF (5 mL) at 0° C. were added TEA (0.80 mL, 5.75 mmol) and 2-nitrobenzenesulfonyl chloride (550 mg, 2.48 mmol) sequentially. The reaction was stirred at 25° C. for 1 h. The mixture was poured into water (30 mL) and extracted with EtOAc (30 mL×3). The combined organic layers were washed with brine (30 mL), dried over anhydrous sodium sulfate, filtered, and concentrated under reduced pressure to afford tert-butyl (2S, 3S)-2-methyl-3-[(2-nitrophenyl)sulfonylamino]pyrrolidine-1-carboxylate (1.20 g, quant. yield) as a yellow oil. LCMS (MM-ES+APCI, Pos): m/z 285.9 (M+H-Boc).

Step B. tert-butyl (2S,3S)-2-methyl-3-((N-methyl-4-nitrophenyl)sulfonamido)pyrrolidine-1-carboxylate. To a solution of tert-butyl (2S, 3S)-2-methyl-3-[(2-nitrophenyl)sulfonylamino]pyrrolidine-1-carboxylate (600 mg, 1.56 mmol) in DMF (6 mL) were added K₂CO₃ (500 mg, 3.62 mmol) and Mel (0.15 mL, 2.41 mmol). The reaction was stirred at 25° C. for 1 h. The mixture was poured into water (30 mL) and extracted with EtOAc (30 mL×3). The combined organic layers were washed with brine (30 mL), dried over anhydrous sodium sulfate, filtered, and concentrated under reduced pressure to afford tert-butyl (2S, 3S)-2-methyl-3-[methyl-(2-nitrophenyl)sulfonyl-amino]pyrrolidine-1-carboxylate 4 (710 mg, quant. yield) as a yellow oil. LCMS (MM-ES+APCI, Pos): m/z 299.8 (M+H-Boc).

Step C. tert-butyl (2S,3S)-2-methyl-3-(methylamino)pyrrolidine-1-carboxylate. To a solution of tert-butyl (2S, 3S)-2-methyl-3-[methyl-(2-nitrophenyl)sulfonyl-amino]pyrrolidine-1-carboxylate (700 mg, 1.75 mmol) in MeCN (5 mL) were added K₂CO₃ (700 mg, 5.06 mmol) and 2-fluorobenzenethiol (0.38 mL, 3.55 mmol). The reaction was stirred at 20° C. for 2 h. The mixture was poured into water (20 mL) and extracted with EtOAc (20 mL×3). The combined organic layers were washed with brine (20 mL), dried over anhydrous sodium sulfate, filtered, and concentrated under reduced pressure. The crude residue was purified by silica gel column chromatography (1% to 20% MeOH/DCM) to afford tert-butyl (2S, 3S)-2-methyl-3-(methylamino) pyrrolidine-1-carboxylate (340 mg, 82% yield) as a yellow oil. LCMS (MM-ES+APCI, Pos): m/z 159.1 (M+H-^{t}Bu).

Intermediates I176-I178 in Table 15 below were prepared according to Example 54 Steps A to C.

TABLE 15
A list of intermediates 1176-1178

| Int. | | | MS API |
|---|---|---|---|
| ID | Structure | Chemical Name | (m/z) |
| I176 | 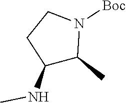 | tert-butyl (2S,3S)-2-methyl-3- (methylamino)pyrrolidine-1- carboxylate | 159.1 (M + H-^{t}Bu) |
| I177 | 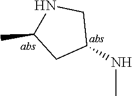 | tert-butyl (2R,4R)-2-methyl-4- (methylamino)pyrrolidine-1- carboxylate | 159.1 (M + H-^{t}Bu) |
| I178 | 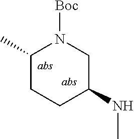 | tert-butyl (2S,5S)-2-methyl-5- (methylamino)piperidine-1- carboxylate | 229.1 (M + H) |

Syntheses for the compounds of Formula (I), (IA)-(ID), (IA-1), (IA-2), (IB-1), or (IB-2) are described below.

### Example 55

### (R)-1-(2-methyl-4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one (compound 1)

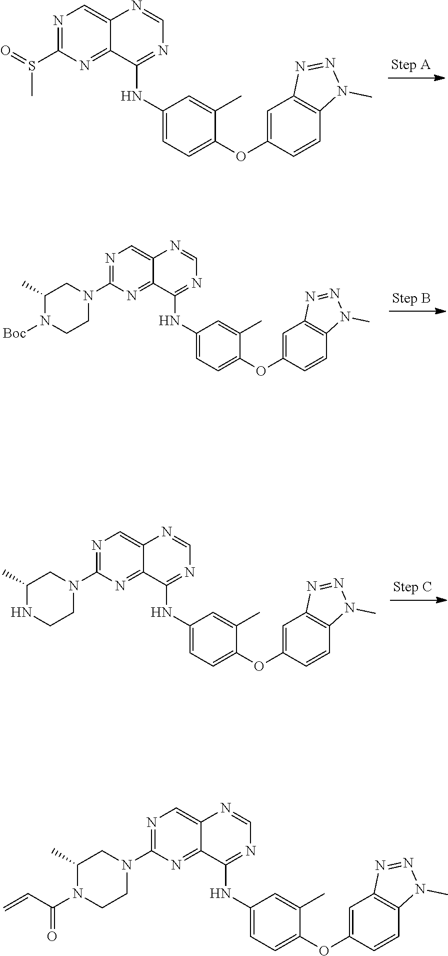

Step A. tert-butyl (R)-2-methyl-4-(8-((3-methyl-4-((1-methyl-1H-benzo[d][1,2,3]triazol-5-yl)oxy)phenyl)amino)pyrimido[5,4-d]pyrimidin-2-yl)piperazine-1-carboxylate. To a solution of N-[3-methyl-4-(1-methylbenzotriazol-5-yl)oxy-phenyl]-6-methylsulfinyl-pyrimido[5,4-d]pyrimidin-4-amine (80 mg, 0.14 mmol) and tert-butyl (R)-2-methylpiperazine-1-carboxylate (42 mg, 0.21 mmol) in IPA (1.5 mL) was added DIPEA (45 mg, 0.35 mmol). The mixture was stirred at 80° C. for 16 h and concentrated under reduced pressure. The crude product was purified by silica gel column chromatography (10% to 100% EtOAc/PET) to afford tert-butyl (R)-2-methyl-4-[4-[3-methyl-4-(1-methylbenzotriazol-5-yl)oxy-anilino]pyrimido[5,4-d]pyrimidin-6-yl]piperazine-1-carboxylate (40 mg, 41% yield) as a yellow oil. LCMS (MM-ES+APCI, Pos): m/z 583.3 (M+H).

Step B. (R)—N-(3-methyl-4-((1-methyl-1H-benzo[d][1,2,3]triazol-5-yl)oxy)phenyl)-6-(3-methylpiperazin-1-yl)pyrimido[5,4-d]pyrimidin-4-amine hydrochloride. To a solution of tert-butyl (R)-2-methyl-4-[4-[3-methyl-4-(1-methylbenzotriazol-5-yl)oxy-anilino]pyrimido[5,4-d]pyrimidin-6-yl]piperazine-1-carboxylate (35 mg, 50 μmol) in DCM (1 mL) was added HCl (4M in 1,4-dioxane, 1 mL, 4.0 mmol). The mixture was stirred at 25° C. for 1 h and concentrated under reduced pressure to afford (R)—N-(3-methyl-4-((1-methyl-1H-benzo[d][1,2,3]triazol-5-yl)oxy)phenyl)-6-(3-methylpiperazin-1-yl)pyrimido[5,4-d]pyrimidin-4-amine hydrochloride (40 mg, quant. yield) as a yellow oil. LCMS (MM-ES+APCI, Pos): m/z 483.3 (M+H—HCl).

Step C. (R)-1-(2-methyl-4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one. To a solution of (R)—N-(3-methyl-4-((1-methyl-1H-benzo[d][1,2,3]triazol-5-yl)oxy)phenyl)-6-(3-methylpiperazin-1-yl)pyrimido[5,4-d]pyrimidin-4-amine hydrochloride (30 mg, 58 μmol) in DMF (1 mL) was added TEA (16 μL, 116 μmol). The mixture was cooled to 0° C. and acryloyl chloride (4.7 μL, 58 μmol) was added dropwise. The reaction was warmed to 25° C. and stirred for 0.5 h, quenched with sat. aq. NaHCO₃ (0.2 mL) and filtered. The filtrate was concentrated in vacuo, and the residue was purified by preparative HPLC (30% to 60% MeCN/0.05% aqueous NH₄HCO₃). Lyophilization afforded (R)-1-(2-methyl-4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one (10 mg, 25% yield) as a white solid. LCMS (MM-ES+APCI, Pos): m/z 537.1 (M+H).

Compounds 1-49 and 111-195 in Table 4 below were prepared according to Example 55 Steps A to C.

Step A can be carried out using IPA, DMF, DMSO, or 1,4-dioxane as solvent. The Boc-deprotection (Step B) can be accomplished equally well using HCl (4M in 1,4-dioxane or EtOAc) or TFA. Step C can employ DMF or DCM interchangeably as solvent. Options for purification include C₁₈ preparative HPLC, silica gel chromatography, and/or preparative TLC.

TABLE 4
A list of compounds 1-49, 111-195

| | | | MS |
|---|---|---|---|
| Cmp. | | | API |
| ID | Structure | Chemical Name | (m/z) |
| 1 | 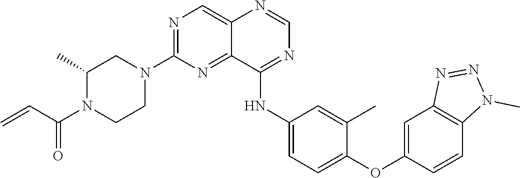 [SMILES: C=CC(=O)N1CCN(c2ncc3ncnc(Nc4ccc(Oc5ccc6c(c5)nnn6C)c(C)c4)c3n2)C[C@H]1C] | (R)-1-(2-methyl-4-(8-((3-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one | 537.1 (M + H) |
| 2 | 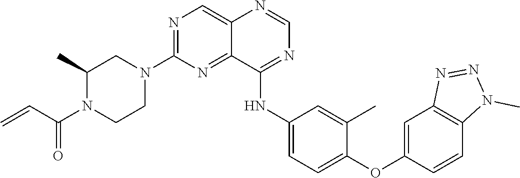 [SMILES: C=CC(=O)N1CCN(c2ncc3ncnc(Nc4ccc(Oc5ccc6c(c5)nnn6C)c(C)c4)c3n2)C[C@@H]1C] | (S)-1-(2-methyl-4-(8-((3-methyl-4- ((1-methyl-1H- benzo[d][1,2,3]triazol-5- yl)oxy)phenyl)amino)pyrimido[5,4- d]pyrimidin-2-y1)piperazin-1- yl)prop-2-en-1-one | 537.1 (M + H) |
| 3 | 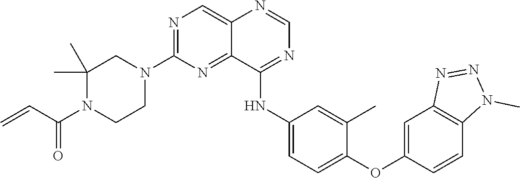 [SMILES: C=CC(=O)N1CCN(c2ncc3ncnc(Nc4ccc(Oc5ccc6c(c5)nnn6C)c(C)c4)c3n2)CC1(C)C] | 1-(2,2-dimethyl-4-(8-((3-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.4 (M + H) |
| 4 | 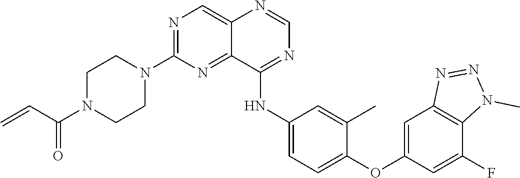 [SMILES: C=CC(=O)N1CCN(c2ncc3ncnc(Nc4ccc(Oc5cc(F)c6c(c5)nnn6C)c(C)c4)c3n2)CC1] | 1-(4-(8-((4-((7-fluoro-1-methyl-1H- benzo[d][1,2,3]triazol-5-yl)oxy)-3- methylphenyl)amino)pyrimido[5,4- d]pyrimidin-2-yl)piperazin-1- yl)prop-2-en-1-one | 541.1 (M + H) |
| 5 | 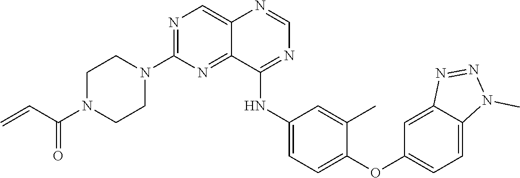 [SMILES: C=CC(=O)N1CCN(c2ncc3ncnc(Nc4ccc(Oc5ccc6c(c5)nnn6C)c(C)c4)c3n2)CC1] | 1-(4-(8-((3-methyl-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 523.3 (M + H) |
| 6 | 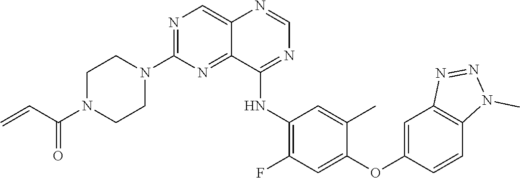 [SMILES: C=CC(=O)N1CCN(c2ncc3ncnc(Nc4cc(C)c(Oc5ccc6c(c5)nnn6C)cc4F)c3n2)CC1] | 1-(4-(8-((2-fluoro-5-methyl-4-((1- methyl-1H-benzo[d][1,2,3]triazol- 5-yl)oxy)phenyl)amino)pyrimido [5,4-d]pyrimidin-2-yl)piperazin-1- yl)prop-2-en-1-one | 541.2 (M + H) |
| 7 | 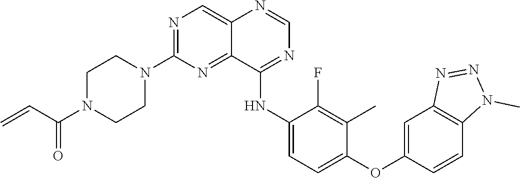 [SMILES: C=CC(=O)N1CCN(c2ncc3ncnc(Nc4ccc(Oc5ccc6c(c5)nnn6C)c(C)c4F)c3n2)CC1] | 1-(4-(8-((2-fluoro-3-methyl-4-((1- methyl-1H-benzo[d][1,2,3]triazol- 5-yl)oxy)phenyl)amino)pyrimido [5,4-d]pyrimidin-2-y1)piperazin-1- yl)prop-2-en-1-one | 541.1 (M + H) |
| 8 | 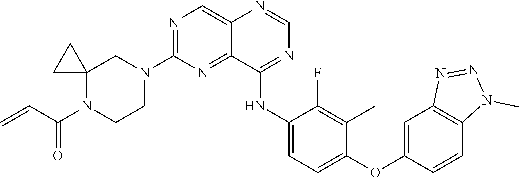 [SMILES: C=CC(=O)N1CCN(c2ncc3ncnc(Nc4ccc(Oc5ccc6c(c5)nnn6C)c(C)c4F)c3n2)CC12CC2] | 1-(7-(8-((2-fluoro-3-methyl-4-((1- methyl-1H-benzo[d][1,2,3]triazol- 5-yl)oxy)phenyl)amino)pyrimido [5,4-d]pyrimidin-2-yl)-4,7- diazaspiro[2.5]octan-4-yl)prop-2- en-1-one | 567.2 (M + H) |
| 9 | 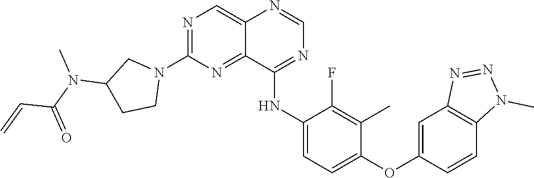 [SMILES: C=CC(=O)N(C)C1CCN(c2ncc3ncnc(Nc4ccc(Oc5ccc6c(c5)nnn6C)c(C)c4F)c3n2)C1] | N-(1-(8-((2-fluoro-3-methyl-4-((1- methyl-1H-benzo[d][1,2,3]triazol- 5-yl)oxy)phenyl)amino)pyrimido [5,4-d]pyrimidin-2-yl)pyrrolidin- 3-y1)-N-methylacrylamide | 555.1 (M + H) |
| 10 | 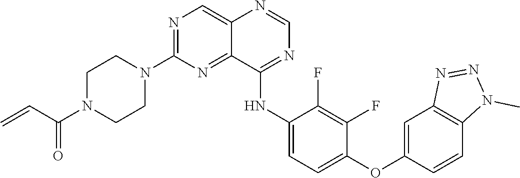 [SMILES: C=CC(=O)N1CCN(c2ncc3ncnc(Nc4ccc(Oc5ccc6c(c5)nnn6C)c(F)c4F)c3n2)CC1] | 1-(4-(8-((2,3-difluoro-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 545.1 (M + H) |
| 11 | 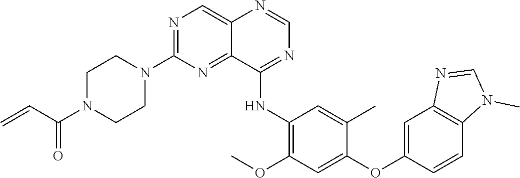 [SMILES: C=CC(=O)N1CCN(c2ncc3ncnc(Nc4cc(C)c(Oc5ccc6c(c5)ncn6C)cc4OC)c3n2)CC1] | 1-(4-(8-((2-methoxy-5-methyl-4- ((1-methyl-1H-benzo[d]imidazol-5- yl)oxy)phenyl)amino)pyrimido[5,4- d]pyrimidin-2-yl)piperazin-1- yl)prop-2-en-1-one 2,2,2- trifluoroacetate | 552.3 (M + H − TFA) |
| 12 | 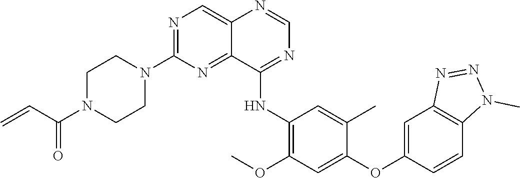 [SMILES: C=CC(=O)N1CCN(c2ncc3ncnc(Nc4cc(C)c(Oc5ccc6c(c5)nnn6C)cc4OC)c3n2)CC1] | 1-(4-(8-((2-methoxy-5-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one 2,2,2- trifluoroacetate | 553.2 (M + H − TFA) |
| 13 | 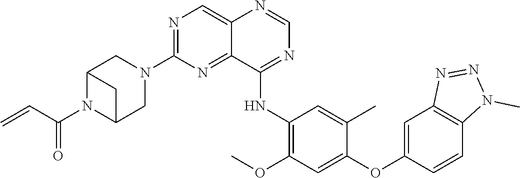 [SMILES: C=CC(=O)N1C2CC1CN(c1ncc3ncnc(Nc4cc(C)c(Oc5ccc6c(c5)nnn6C)cc4OC)c3n1)C2] | 1-(3-(8-((2-methoxy-5-methyl-4- ((1-methyl-1H- benzo[d][1,2,3]triazol-5- yl)oxy)phenyl)amino)pyrimido[5,4- d]pyrimidin-2-y1)-3,6- diazabicyclo[3.1.1]heptan-6- yl)prop-2-en-1-one | 565.3 (M + H) |
| 14 | 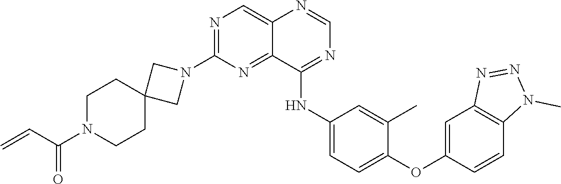 [SMILES: C=CC(=O)N1CCC2(CC1)CN(c1ncc3ncnc(Nc4ccc(Oc5ccc6c(c5)nnn6C)c(C)c4)c3n1)C2] | 1-(2-(8-((3-methyl-4-((1-methyl- 1H-benzo[d][1,2,3]triazol-5- yl)oxy)phenyl)amino)pyrimido[5,4- d]pyrimidin-2-y1)-2,7- diazaspiro[3.5]nonan-7-yl)prop-2- en-1-one | 563.3 (M + H) |
| 15 | 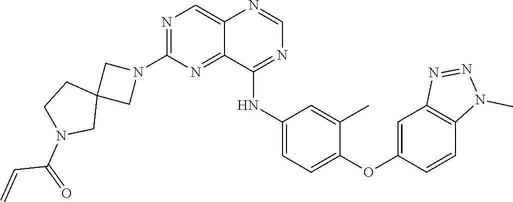 [SMILES: C=CC(=O)N1CCC2(C1)CN(c1ncc3ncnc(Nc4ccc(Oc5ccc6c(c5)nnn6C)c(C)c4)c3n1)C2] | 1-(2-(8-((3-methyl-4-((1-methyl- 1H-benzo[d][1,2,3]triazol-5- yl)oxy)phenyl)amino)pyrimido[5,4- d]pyrimidin-2-yl)-2,6- diazaspiro[3.4]octan-6-yl)prop-2- en-1-one | 549.3 (M + H) |
| 16 | 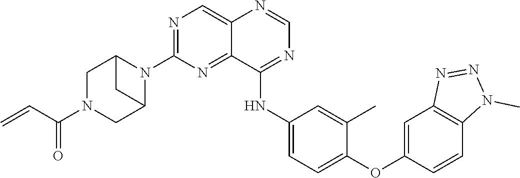 [SMILES: C=CC(=O)N1CC2CC(C1)N2c1ncc2ncnc(Nc3ccc(Oc4ccc5c(c4)nnn5C)c(C)c3)c2n1] | 1-(6-(8-((3-methyl-4-((1-methyl- 1H-benzo[d][1,2,3]triazol-5- yl)oxy)phenyl)amino)pyrimido[5,4- d]pyrimidin-2-yl)-3,6- diazabicyclo[3.1.1]heptan-3- yl)prop-2-en-1-one | 535.2 (M + H) |
| 17 | 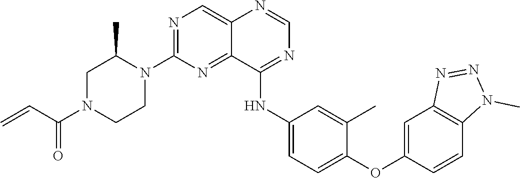 [SMILES: C=CC(=O)N1CCN(c2ncc3ncnc(Nc4ccc(Oc5ccc6c(c5)nnn6C)c(C)c4)c3n2)[C@H](C)C1] | (R)-1-(3-methyl-4-(8-((3-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one | 537.3 (M + H) |
| 18 | 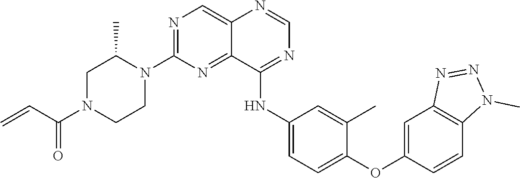 [SMILES: C=CC(=O)N1CCN(c2ncc3ncnc(Nc4ccc(Oc5ccc6c(c5)nnn6C)c(C)c4)c3n2)[C@@H](C)C1] | (S)-1-(3-methyl-4-(8-((3-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one 2,2,2- trifluoroacetate | 537.3 (M + H − TFA) |
| 19 | 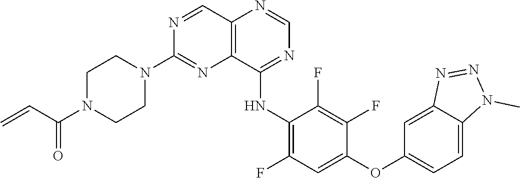 [SMILES: C=CC(=O)N1CCN(c2ncc3ncnc(Nc4c(F)cc(Oc5ccc6c(c5)nnn6C)c(F)c4F)c3n2)CC1] | 1-(4-(8-((2,3,6-trifluoro-4-((1- methyl-1H-benzo[d][1,2,3]triazol- 5-yl)oxy)phenyl)amino)pyrimido [5,4-d]pyrimidin-2-yl)piperazin-1- yl)prop-2-en-1-one | 563.2 (M + H) |
| 20 | 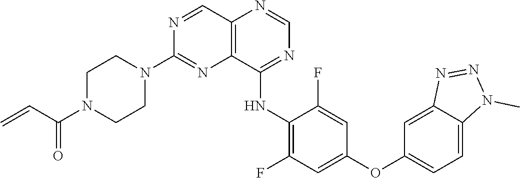 [SMILES: C=CC(=O)N1CCN(c2ncc3ncnc(Nc4c(F)cc(Oc5ccc6c(c5)nnn6C)cc4F)c3n2)CC1] | 1-(4-(8-((2,6-difluoro-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 545.2 (M + H) |
| 21 | 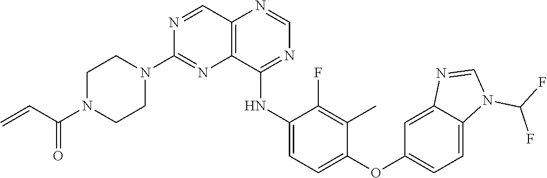 [SMILES: C=CC(=O)N1CCN(c2ncc3ncnc(Nc4ccc(Oc5ccc6c(c5)ncn6C(F)F)c(C)c4F)c3n2)CC1] | 1-(4-(8-((4-((1-(difluoromethyl)- 1H-benzo[d]imidazol-5-y1)oxy)-2- fluoro-3- methylphenyl)amino)pyrimido[5,4- d]pyrimidin-2-yl)piperazin-1- yl)prop-2-en-1-one | 576.4 (M + H) |
| 22 | 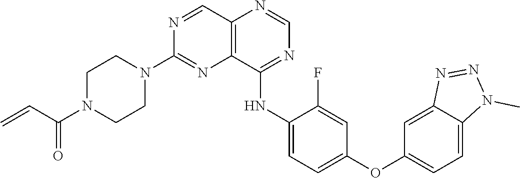 [SMILES: C=CC(=O)N1CCN(c2ncc3ncnc(Nc4ccc(Oc5ccc6c(c5)nnn6C)cc4F)c3n2)CC1] | 1-(4-(8-((2-fluoro-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 527.2 (M + H) |
| 23 | 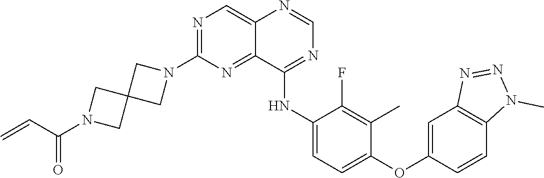 [SMILES: C=CC(=O)N1CC2(C1)CN(c1ncc3ncnc(Nc4ccc(Oc5ccc6c(c5)nnn6C)c(C)c4F)c3n1)C2] | 1-(6-(8-((2-fluoro-3-methyl-4-((1- methyl-1H-benzo[d][1,2,3]triazol- 5-yl)oxy)phenyl)amino)pyrimido [5,4-d]pyrimidin-2-yl)-2,6- diazaspiro[3.3]heptan-2-yl)prop-2- en-1-one | 553.3 (M + H) |
| 24 | 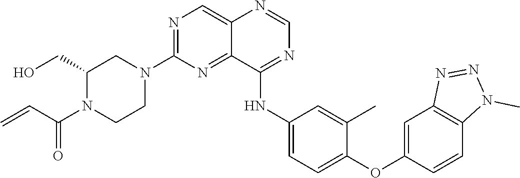 [SMILES: C=CC(=O)N1CCN(c2ncc3ncnc(Nc4ccc(Oc5ccc6c(c5)nnn6C)c(C)c4)c3n2)C[C@H]1CO] | (S)-1-(2-(hydroxymethyl)-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 553.4 (M + H) |
| 25 | 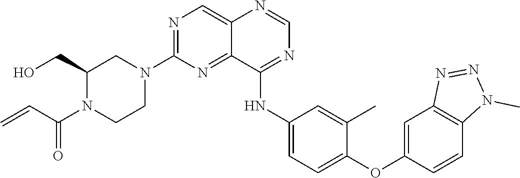 [SMILES: C=CC(=O)N1CCN(c2ncc3ncnc(Nc4ccc(Oc5ccc6c(c5)nnn6C)c(C)c4)c3n2)C[C@@H]1CO] | (R)-1-(2-(hydroxymethyl)-4-(8-((3- methyl-4-((1-methyl-1H- benzo[d][1,2,3]triazol-5- yl)oxy)phenyl)amino)pyrimido[5,4- d]pyrimidin-2-y1)piperazin-1- yl)prop-2-en-1-one | 553.4 (M + H) |
| 26 | 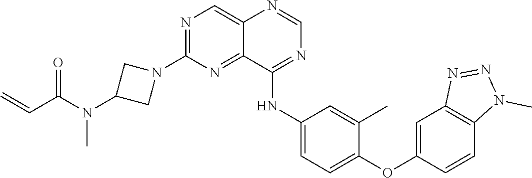 [SMILES: C=CC(=O)N(C)C1CN(c2ncc3ncnc(Nc4ccc(Oc5ccc6c(c5)nnn6C)c(C)c4)c3n2)C1] | N-methyl-N-(1-(8-((3-methyl-4- ((1-methyl-1H- benzo[d][1,2,3]triazol-5- yl)oxy)phenyl)amino)pyrimido[5,4- d]pyrimidin-2-yl)azetidin-3- yl)acrylamide | 523.3 (M + H) |
| 27 | 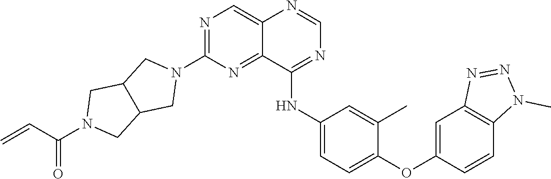 [SMILES: C=CC(=O)N1CC2CN(c3ncc4ncnc(Nc5ccc(Oc6ccc7c(c6)nnn7C)c(C)c5)c4n3)CC2C1] | 1-(5-(8-((3-methyl-4-((1-methyl- 1H-benzo[d][1,2,3]triazol-5- yl)oxy)phenyl)amino)pyrimido[5,4- d]pyrimidin-2- yl)hexahydropyrrolo[3,4-c]pyrrol- 2(1H)-y1)prop-2-en-1-one | 549.2 (M + H) |
| 28 | 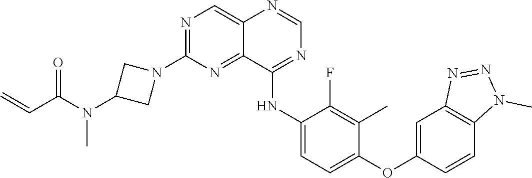 [SMILES: C=CC(=O)N(C)C1CN(c2ncc3ncnc(Nc4ccc(Oc5ccc6c(c5)nnn6C)c(C)c4F)c3n2)C1] | N-(1-(8-((2-fluoro-3-methyl-4-((1- methyl-1H-benzo[d][1,2,3]triazol- 5-yl)oxy)phenyl)amino)pyrimido [5,4-d]pyrimidin-2-yl)azetidin- 3-yl)-N-methylacrylamide | 541.2 (M + H) |
| 29 | 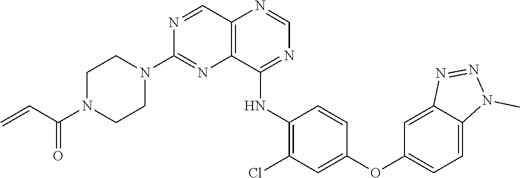 [SMILES: C=CC(=O)N1CCN(c2ncc3ncnc(Nc4ccc(Oc5ccc6c(c5)nnn6C)cc4Cl)c3n2)CC1] | 1-(4-(8-((2-chloro-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 543.3 (M + H) |
| 30 | 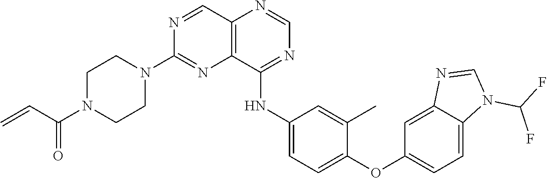 [SMILES: C=CC(=O)N1CCN(c2ncc3ncnc(Nc4ccc(Oc5ccc6c(c5)ncn6C(F)F)c(C)c4)c3n2)CC1] | 1-(4-(8-((4-((1-(difluoromethyl)- 1H-benzo[d]imidazol-5-yl)oxy)-3- methylphenyl)amino)pyrimido[5,4- d]pyrimidin-2-y1)piperazin-1- yl)prop-2-en-1-one | 558.4 (M + H) |
| 31 | 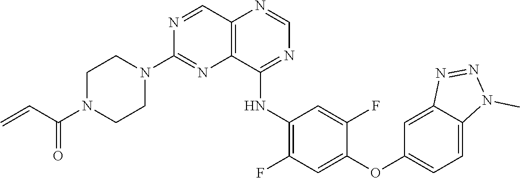 [SMILES: C=CC(=O)N1CCN(c2ncc3ncnc(Nc4cc(F)c(Oc5ccc6c(c5)nnn6C)cc4F)c3n2)CC1] | 1-(4-(8-((2,5-difluoro-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 545.2 (M + H) |
| 32 | 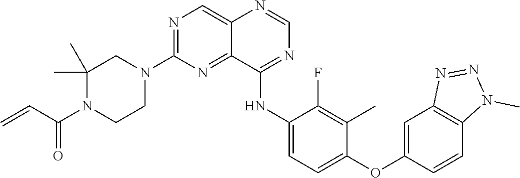 [SMILES: C=CC(=O)N1CCN(c2ncc3ncnc(Nc4ccc(Oc5ccc6c(c5)nnn6C)c(C)c4F)c3n2)CC1(C)C] | 1-(4-(8-((2-fluoro-3-methyl-4-((1- methyl-1H-benzo[d][1,2,3]triazol- 5-yl)oxy)phenyl)amino)pyrimido [5,4-d]pyrimidin-2-yl)-2,2- dimethylpiperazin-1-yl)prop-2-en- 1-one | 569.3 (M + H) |
| 33 | 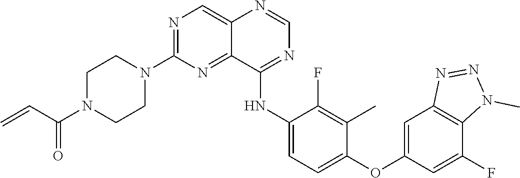 [SMILES: C=CC(=O)N1CCN(c2ncc3ncnc(Nc4ccc(Oc5cc(F)c6c(c5)nnn6C)c(C)c4F)c3n2)CC1] | 1-(4-(8-((2-fluoro-4-((7-fluoro-1- methyl-1H-benzo[d][1,2,3]triazol- 5-yl)oxy)-3- methylphenyl)amino)pyrimido [5,4-d]pyrimidin-2-yl)piperazin-1- yl)prop-2-en-1-one | 559.2 (M + H) |
| 34 | 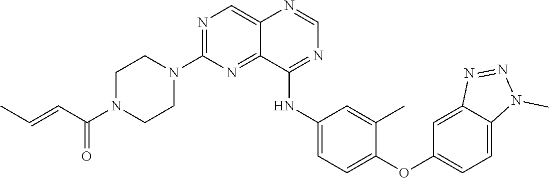 [SMILES: C/C=C/C(=O)N1CCN(c2ncc3ncnc(Nc4ccc(Oc5ccc6c(c5)nnn6C)c(C)c4)c3n2)CC1] | (E)-1-(4-(8-((3-methyl-4-((1- methyl-1H-benzo[d][1,2,3]triazol- 5-yl)oxy)phenyl)amino)pyrimido [5,4-d]pyrimidin-2-yl)piperazin-1- yl)but-2-en-1-one | 537.5 (M + H) |
| 35 | 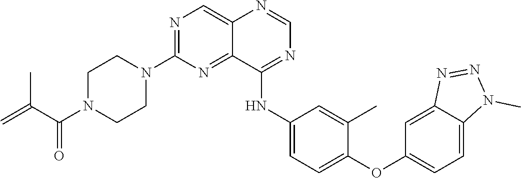 [SMILES: C=C(C)C(=O)N1CCN(c2ncc3ncnc(Nc4ccc(Oc5ccc6c(c5)nnn6C)c(C)c4)c3n2)CC1] | 2-methyl-1-(4-(8-((3-methyl-4-((1- methyl-1H-benzo[d][1,2,3]triazol- 5-yl)oxy)phenyl)amino)pyrimido [5,4-d]pyrimidin-2-y1)piperazin-1- yl)prop-2-en-1-one | 537.5 (M + H) |
| 36 | 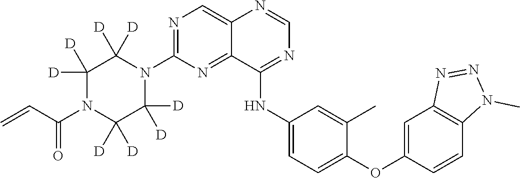 [SMILES: [2H]C1([2H])N(C(=O)C=C)C([2H])([2H])C([2H])([2H])N(c2ncc3ncnc(Nc4ccc(Oc5ccc6c(c5)nnn6C)c(C)c4)c3n2)C1([2H])[2H]] | 1-(4-(8-((3-methyl-4-((1-methyl- 1H-benzo[d][1,2,3]triazol-5- yl)oxy)phenyl)amino)pyrimido[5,4- d]pyrimidin-2-yl)piperazin-1-yl- 2,2,3,3,5,5,6,6-d8)prop-2-en-1-one | 531.4 (M + H) |
| 37 | 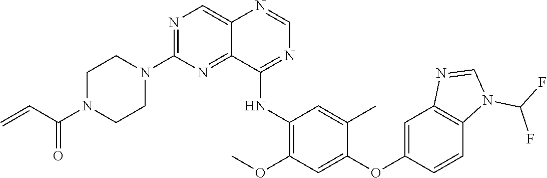 [SMILES: C=CC(=O)N1CCN(c2ncc3ncnc(Nc4cc(C)c(Oc5ccc6c(c5)ncn6C(F)F)cc4OC)c3n2)CC1] | 1-(4-(8-((4-((1-(difluoromethyl)- 1H-benzo[d]imidazol-5-y1)oxy)-2- methoxy-5- methylphenyl)amino)pyrimido[5,4- d]pyrimidin-2-yl)piperazin-1- yl)prop-2-en-1-one 2,2,2- trifluoroacetate | 588.2 (M + H − TFA) |
| 38 | 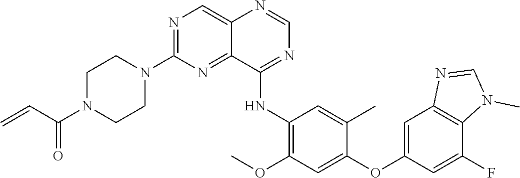 [SMILES: C=CC(=O)N1CCN(c2ncc3ncnc(Nc4cc(C)c(Oc5cc(F)c6c(c5)ncn6C)cc4OC)c3n2)CC1] | 1-(4-(8-((4-((7-fluoro-1-methyl-1H- benzo[d]imidazol-5-y1)oxy)-2- methoxy-5- methylphenyl)amino)pyrimido[5,4- d]pyrimidin-2-yl)piperazin-1- yl)prop-2-en-1-one 2,2,2- trifluoroacetate | 570.3 (M + H − TFA) |
| 39 | 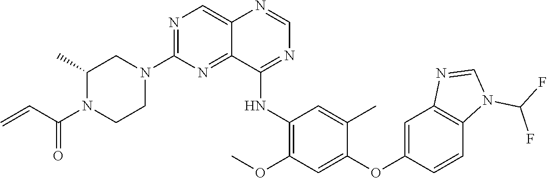 [SMILES: C=CC(=O)N1CCN(c2ncc3ncnc(Nc4cc(C)c(Oc5ccc6c(c5)ncn6C(F)F)cc4OC)c3n2)C[C@H]1C] | (R)-1-(4-(8-((4-((1- (difluoromethyl)-1H- benzo[d]imidazol-5-yl)oxy)-2- methoxy-5- methylphenyl)amino)pyrimido[5,4- d]pyrimidin-2-y1)-2- methylpiperazin-1-yl)prop-2-en-1- one | 602.3 (M + H) |
| 40 | 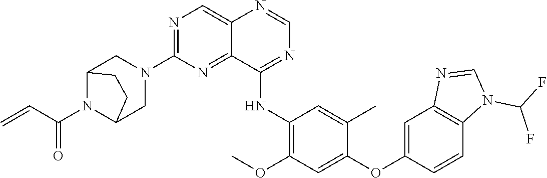 [SMILES: C=CC(=O)N1C2CCC1CN(c1ncc3ncnc(Nc4cc(C)c(Oc5ccc6c(c5)ncn6C(F)F)cc4OC)c3n1)C2] | 1-(3-(8-((4-((1-(difluoromethyl)- 1H-benzo[d]imidazol-5-yl)oxy)-2- methoxy-5- methylphenyl)amino)pyrimido[5,4- d]pyrimidin-2-yl)-3,8- diazabicyclo[3.2.1]octan-8-yl)prop- 2-en-1-one 2,2,2-trifluoroacetate | 614.3 (M + H − TFA) |
| 41 | 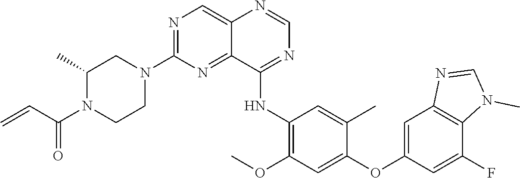 [SMILES: C=CC(=O)N1CCN(c2ncc3ncnc(Nc4cc(C)c(Oc5cc(F)c6c(c5)ncn6C)cc4OC)c3n2)C[C@H]1C] | (R)-1-(4-(8-((4-((7-fluoro-1- methyl-1H-benzo[d]imidazol-5- yl)oxy)-2-methoxy-5- methylphenyl)amino)pyrimido[5,4- d]pyrimidin-2-y1)-2- methylpiperazin-1-y1)prop-2-en-1- one | 584.3 (M + H) |
| 42 | 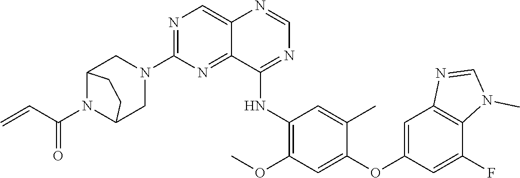 [SMILES: C=CC(=O)N1C2CCC1CN(c1ncc3ncnc(Nc4cc(C)c(Oc5cc(F)c6c(c5)ncn6C)cc4OC)c3n1)C2] | 1-(3-(8-((4-((7-fluoro-1-methyl-1H- benzo[d]imidazol-5-yl)oxy)-2- methoxy-5- methylphenyl)amino)pyrimido[5,4- d]pyrimidin-2-y1)-3,8- diazabicyclo[3.2.1]octan-8-yl)prop- 2-en-1-one | 596.3 (M + H) |
| 43 | 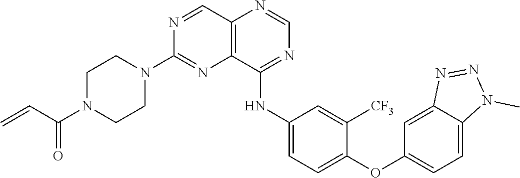 [SMILES: C=CC(=O)N1CCN(c2ncc3ncnc(Nc4ccc(Oc5ccc6c(c5)nnn6C)c(C(F)(F)F)c4)c3n2)CC1] | 1-(4-(8-((4-((1-methyl-1H- benzo[d][1,2,3]triazol-5-yl)oxy)-3- (trifluoromethyl)phenyl)amino)pyri mido[5,4-d]pyrimidin-2- yl)piperazin-1-yl)prop-2-en-1-one | 577.3 (M + H) |
| 44 | 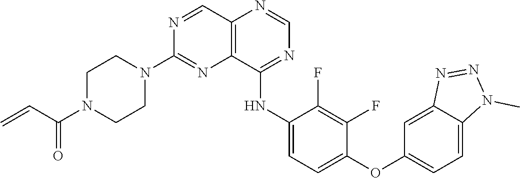 [SMILES: C=CC(=O)N1CCN(c2ncc3ncnc(Nc4ccc(Oc5ccc6c(c5)nnn6C)c(F)c4F)c3n2)CC1] | 1-(4-(8-((2,3-difluoro-4-((1-methyl- 1H-benzo[d][1,2,3]triazol-5- yl)oxy)phenyl)amino)pyrimido[5,4- d]pyrimidin-2-yl)piperazin-1- yl)prop-2-en-1-one | 545.2 (M + H) |
| 45 | 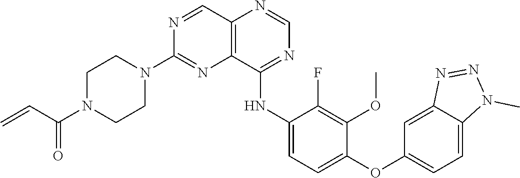 [SMILES: C=CC(=O)N1CCN(c2ncc3ncnc(Nc4ccc(Oc5ccc6c(c5)nnn6C)c(OC)c4F)c3n2)CC1] | 1-(4-(8-((2-fluoro-3-methoxy-4-((1- methyl-1H-benzo[d][1,2,3]triazol- 5-yl)oxy)phenyl)amino)pyrimido [5,4-d]pyrimidin-2-yl)piperazin-1- yl)prop-2-en-1-one | 557.3 (M + H) |
| 46 | 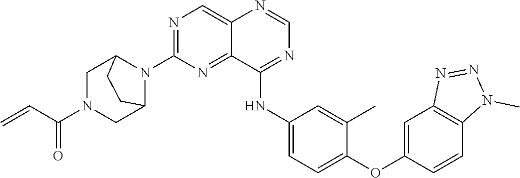 [SMILES: C=CC(=O)N1CC2CCC(C1)N2c1ncc2ncnc(Nc3ccc(Oc4ccc5c(c4)nnn5C)c(C)c3)c2n1] | 1-(8-(8-((3-methyl-4-((1-methyl- 1H-benzo[d][1,2,3]triazol-5- yl)oxy)phenyl)amino)pyrimido[5,4- d]pyrimidin-2-yl)-3,8- diazabicyclo[3.2.1]octan-3-yl)prop- 2-en-1-one | 549.2 (M + H) |
| 47 | 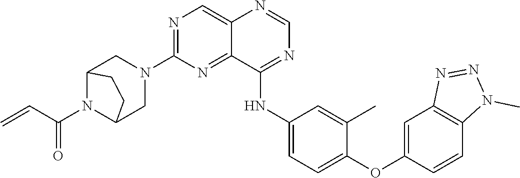 [SMILES: C=CC(=O)N1C2CCC1CN(c1ncc3ncnc(Nc4ccc(Oc5ccc6c(c5)nnn6C)c(C)c4)c3n1)C2] | 1-(3-(8-((3-methyl-4-((1-methyl- 1H-benzo[d][1,2,3]triazol-5- yl)oxy)phenyl)amino)pyrimido[5,4- d]pyrimidin-2-yl)-3,8- diazabicyclo[3.2.1]octan-8-yl)prop- 2-en-1-one | 549.3 (M + H) |
| 48 | 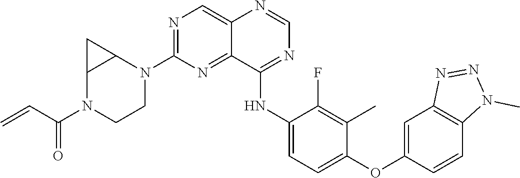 [SMILES: C=CC(=O)N1CCN(c2ncc3ncnc(Nc4ccc(Oc5ccc6c(c5)nnn6C)c(C)c4F)c3n2)C2CC21] | 1-(5-(8-((2-fluoro-3-methyl-4-((1- methyl-1H-benzo[d][1,2,3]triazol- 5- yl)oxy)phenyl)amino)pyrimido[5,4- d]pyrimidin-2-yl)-2,5- diazabicyclo[4.1.0]heptan-2- yl)prop-2-en-1-one | 553.3 (M + H) |
| 49 | 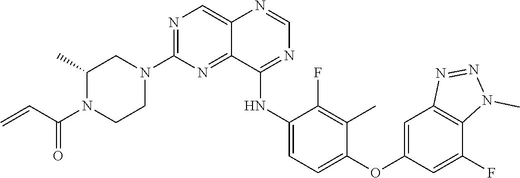 [SMILES: C=CC(=O)N1CCN(c2ncc3ncnc(Nc4ccc(Oc5cc(F)c6c(c5)nnn6C)c(C)c4F)c3n2)C[C@H]1C] | (R)-1-(4-(8-((2-fluoro-4-((7-fluoro- 1-methyl-1H- benzo[d][1,2,3]triazol-5-yl)oxy)-3- methylphenyl)amino)pyrimido[5,4- d]pyrimidin-2-yl)-2- methylpiperazin-1-yl)prop-2-en-1- one | 573.3 (M + H) |
| 111 | 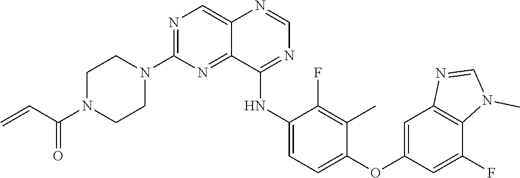 [SMILES: C=CC(=O)N1CCN(c2ncc3ncnc(Nc4ccc(Oc5cc(F)c6c(c5)ncn6C)c(C)c4F)c3n2)CC1] | 1-(4-(8-((2-fluoro-4-((7-fluoro-1- methyl-1H-benzo[d]imidazol-5- yl)oxy)-3- methylphenyl)amino)pyrimido[5,4- d]pyrimidin-2-yl)piperazin-1- y1)prop-2-en-1-one | 558.1 (M + H) |
| 112 | 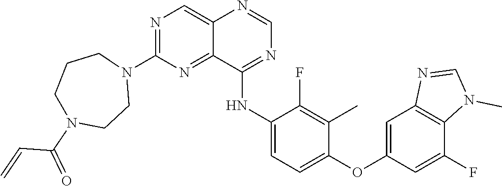 [SMILES: C=CC(=O)N1CCCN(c2ncc3ncnc(Nc4ccc(Oc5cc(F)c6c(c5)ncn6C)c(C)c4F)c3n2)CC1] | 1-(4-(8-((2-fluoro-4-((7-fluoro-1- methyl-1H-benzo[d]imidazol-5- yl)oxy)-3- methylphenyl)amino)pyrimido[5,4- d]pyrimidin-2-yl)-1,4-diazepan-1- yl)prop-2-en-1-one | 572.2 (M + H) |
| 113 | 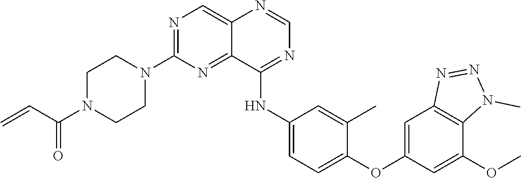 [SMILES: C=CC(=O)N1CCN(c2ncc3ncnc(Nc4ccc(Oc5cc(OC)c6c(c5)nnn6C)c(C)c4)c3n2)CC1] | 1-(4-(8-((4-((7-methoxy-1-methyl- 1H-benzo[d][1,2,3]triazol-5- yl)oxy)-3- methylphenyl)amino)pyrimido[5,4- d]pyrimidin-2-yl)piperazin-1- yl)prop-2-en-1-one | 553.4 (M + H) |
| 114 | 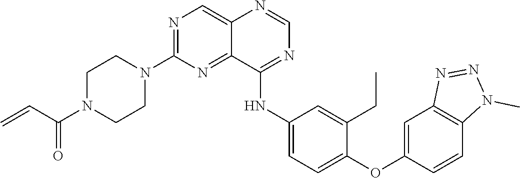 [SMILES: C=CC(=O)N1CCN(c2ncc3ncnc(Nc4ccc(Oc5ccc6c(c5)nnn6C)c(CC)c4)c3n2)CC1] | 1-(4-(8-((3-ethyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 537.3 (M + H) |
| 115 | 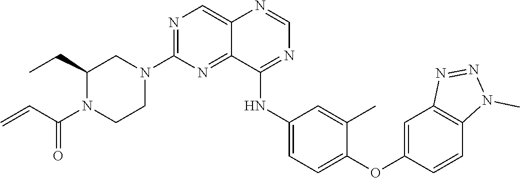 [SMILES: C=CC(=O)N1CCN(c2ncc3ncnc(Nc4ccc(Oc5ccc6c(c5)nnn6C)c(C)c4)c3n2)C[C@@H]1CC] | (S)-1-(2-ethyl-4-(8-((3-methyl-4- ((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 116 | 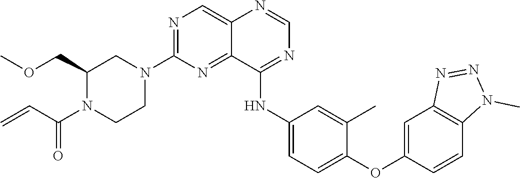 [SMILES: C=CC(=O)N1CCN(c2ncc3ncnc(Nc4ccc(Oc5ccc6c(c5)nnn6C)c(C)c4)c3n2)C[C@@H]1COC] | (R)-1-(2-(methoxymethyl)-4-(8-((3- methyl-4-((1-methyl-1H- benzo[d][1,2,3]triazol-5- yl)oxy)phenyl)amino)pyrimido[5,4- d]pyrimidin-2-y1)piperazin-1- yl)prop-2-en-1-one | 567.2 (M + H) |
| 117 | 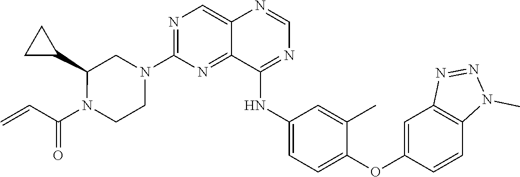 [SMILES: C=CC(=O)N1CCN(c2ncc3ncnc(Nc4ccc(Oc5ccc6c(c5)nnn6C)c(C)c4)c3n2)C[C@@H]1C1CC1] | (S)-1-(2-cyclopropyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 563.3 (M + H) |
| 118 | 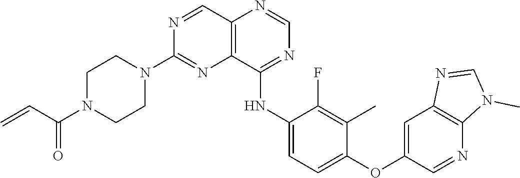 [SMILES: C=CC(=O)N1CCN(c2ncc3ncnc(Nc4ccc(Oc5cnc6c(c5)ncn6C)c(C)c4F)c3n2)CC1] | 1-(4-(8-((2-fluoro-3-methyl-4-((3- methyl-3H-imidazo[4,5-b]pyridin- 6-yl)oxy)phenyl)amino)pyrimido [5,4-d]pyrimidin-2-y1)piperazin-1- yl)prop-2-en-1-one | 541.4 (M + H) |
| 119 | 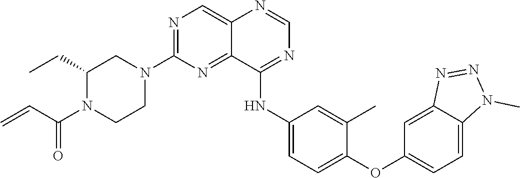 [SMILES: C=CC(=O)N1CCN(c2ncc3ncnc(Nc4ccc(Oc5ccc6c(c5)nnn6C)c(C)c4)c3n2)C[C@H]1CC] | (R)-1-(2-ethyl-4-(8-((3-methyl-4- ((1-methyl-1H- benzo[d][1,2,3]triazol-5- yl)oxy)phenyl)amino)pyrimido[5,4- d]pyrimidin-2-y1)piperazin-1- yl)prop-2-en-1-one | 551.5 (M + H) |
| 120 | 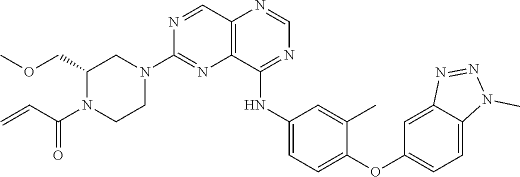 [SMILES: C=CC(=O)N1CCN(c2ncc3ncnc(Nc4ccc(Oc5ccc6c(c5)nnn6C)c(C)c4)c3n2)C[C@H]1COC] | (S)-1-(2-(methoxymethyl)-4-(8-((3- methyl-4-((1-methyl-1H- benzo[d][1,2,3]triazol-5- yl)oxy)phenyl)amino)pyrimido[5,4- d]pyrimidin-2-y1)piperazin-1- yl)prop-2-en-1-one | 567.3 (M + H) |
| 121 | 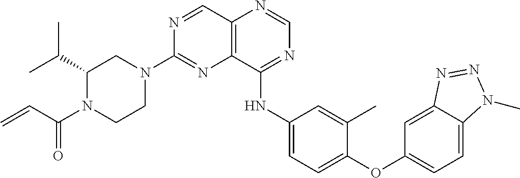 [SMILES: C=CC(=O)N1CCN(c2ncc3ncnc(Nc4ccc(Oc5ccc6c(c5)nnn6C)c(C)c4)c3n2)C[C@H]1C(C)C] | (R)-1-(2-isopropyl-4-(8-((3-methyl- 4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 565.2 (M + H) |
| 122 | 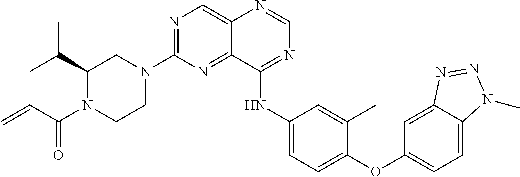 [SMILES: C=CC(=O)N1CCN(c2ncc3ncnc(Nc4ccc(Oc5ccc6c(c5)nnn6C)c(C)c4)c3n2)C[C@@H]1C(C)C] | (S)-1-(2-isopropyl-4-(8-((3-methyl- 4-((1-methyl-1H- benzo[d][1,2,3]triazol-5- yl)oxy)phenyl)amino)pyrimido[5,4- d]pyrimidin-2-yl)piperazin-1- y1)prop-2-en-1-one | 565.2 (M + H) |
| 123 | 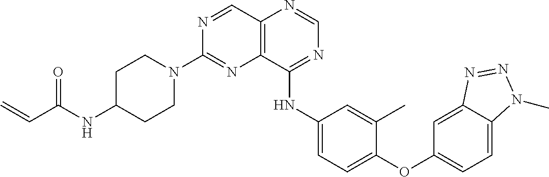 [SMILES: C=CC(=O)NC1CCN(c2ncc3ncnc(Nc4ccc(Oc5ccc6c(c5)nnn6C)c(C)c4)c3n2)CC1] | N-(1-(8-((3-methyl-4-((1-methyl- 1H-benzo[d][1,2,3]triazol-5- yl)oxy)phenyl)amino)pyrimido[5,4- d]pyrimidin-2-yl)piperidin-4- yl)acrylamide | 537.4 (M + H) |
| 124 | 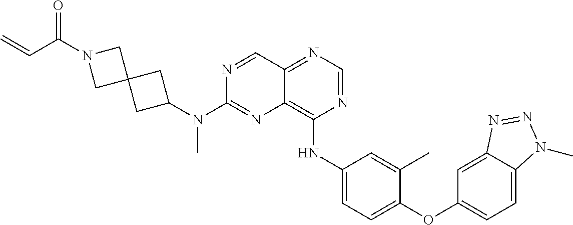 [SMILES: C=CC(=O)N1CC2(CC(N(C)c3ncc4ncnc(Nc5ccc(Oc6ccc7c(c6)nnn7C)c(C)c5)c4n3)C2)C1] | 1-(6-(methyl(8-((3-methyl-4-((1- methyl-1H-benzo[d][1,2,3]triazol- 5-yl)oxy)phenyl)amino)pyrimido [5,4-d]pyrimidin-2-yl)amino)-2- azaspiro[3.3]heptan-2-yl)prop-2-en- 1-one | 563.2 (M + H) |
| 125 | 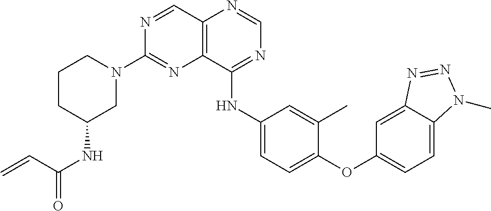 [SMILES: C=CC(=O)N[C@@H]1CCCN(c2ncc3ncnc(Nc4ccc(Oc5ccc6c(c5)nnn6C)c(C)c4)c3n2)C1] | (R)-N-(1-(8-((3-methyl-4-((1- methyl-1H-benzo[d][1,2,3]triazol- 5-yl)oxy)phenyl)amino)pyrimido [5,4-d]pyrimidin-2-y1)piperidin-3- yl)acrylamide | 537.5 (M + H) |
| 126 | 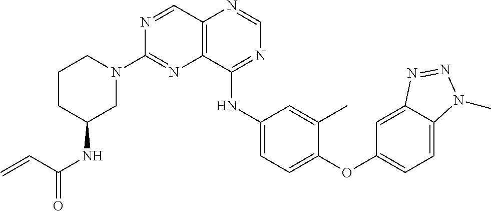 [SMILES: C=CC(=O)N[C@H]1CCCN(c2ncc3ncnc(Nc4ccc(Oc5ccc6c(c5)nnn6C)c(C)c4)c3n2)C1] | (S)-N-(1-(8-((3-methyl-4-((1- methyl-1H-benzo[d][1,2,3]triazol- 5-yl)oxy)phenyl)amino)pyrimido [5,4-d]pyrimidin-2-yl)piperidin-3- yl)acrylamide | 537.3 (M + H) |
| 127 | 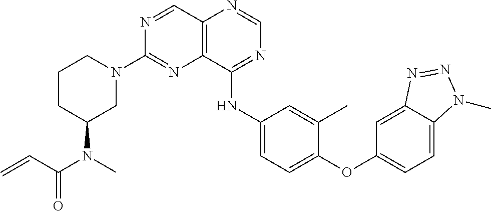 [SMILES: C=CC(=O)N(C)[C@H]1CCCN(c2ncc3ncnc(Nc4ccc(Oc5ccc6c(c5)nnn6C)c(C)c4)c3n2)C1] | (S)-N-methyl-N-(1-(8-((3-methyl- 4-((1-methyl-1H- benzo[d][1,2,3]triazol-5- yl)oxy)phenyl)amino)pyrimido[5,4- d]pyrimidin-2-y1)piperidin-3- yl)acrylamide | 551.2 (M + H) |
| 128 | 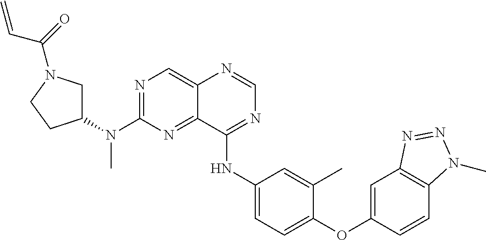 | (R)-1-(3-(methyl(8-((3-methyl-4- ((1-methyl-1H- benzo[d][1,2,3]triazol-5- yl)oxy)phenyl)amino)pyrimido[5,4- d]pyrimidin-2-yl)amino)pyrrolidin- 1-yl)prop-2-en-1-one | 537.3 (M + H) |
| 129 | 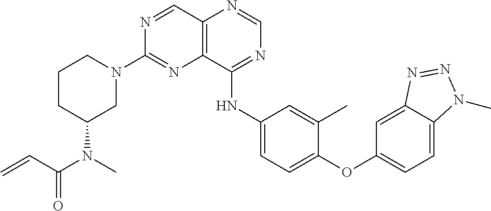 [SMILES: C=CC(=O)N(C)[C@@H]1CCCN(c2ncc3ncnc(Nc4ccc(Oc5ccc6c(c5)nnn6C)c(C)c4)c3n2)C1] | (R)-N-methyl-N-(1-(8-((3-methyl- 4-((1-methyl-1H- benzo[d][1,2,3]triazol-5- yl)oxy)phenyl)amino)pyrimido[5,4- d]pyrimidin-2-y1)piperidin-3- yl)acrylamide | 551.3 (M + H) |
| 130 | 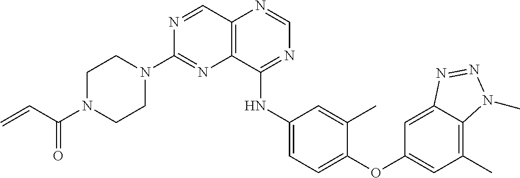 [SMILES: C=CC(=O)N1CCN(c2ncc3ncnc(Nc4ccc(Oc5cc(C)c6c(c5)nnn6C)c(C)c4)c3n2)CC1] | 1-(4-(8-((4-((1,7-dimethyl-1H- benzo[d][1,2,3]triazol-5-yl)oxy)-3- methylphenyl)amino)pyrimido[5,4- d]pyrimidin-2-yl)piperazin-1- yl)prop-2-en-1-one | 537.3 (M + H) |
| 131 | 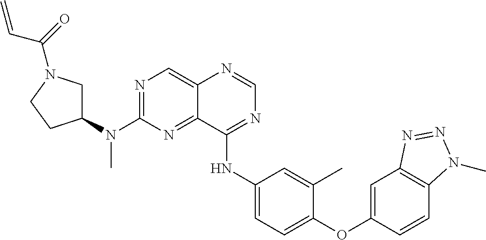 [SMILES: C=CC(=O)N1CC[C@H](N(C)c2ncc3ncnc(Nc4ccc(Oc5ccc6c(c5)nnn6C)c(C)c4)c3n2)C1] | (S)-1-(3-(methyl(8-((3-methyl-4- ((1-methyl-1H- benzo[d][1,2,3]triazol-5- yl)oxy)phenyl)amino)pyrimido[5,4- d]pyrimidin-2-yl)amino)pyrrolidin- 1-yl)prop-2-en-1-one | 537.3 (M + H) |
| 132 | 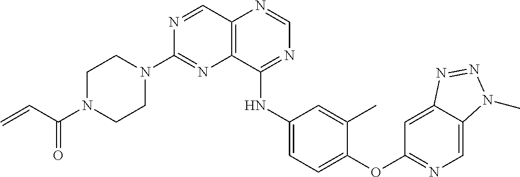 [SMILES: C=CC(=O)N1CCN(c2ncc3ncnc(Nc4ccc(Oc5cc6nnn(C)c6cn5)c(C)c4)c3n2)CC1] | 1-(4-(8-((3-methyl-4-((3-methyl- 3H-[1,2,3]triazolo[4,5-c]pyridin-6- yl)oxy)phenyl)amino)pyrimido[5,4- d]pyrimidin-2-yl)piperazin-1- yl)prop-2-en-1-one | 524.2 (M + H) |
| 133 | 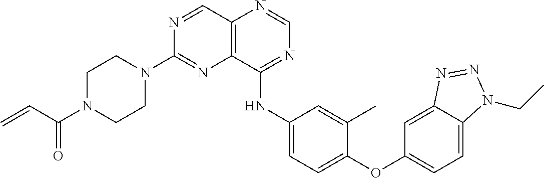 [SMILES: C=CC(=O)N1CCN(c2ncc3ncnc(Nc4ccc(Oc5ccc6c(c5)nnn6CC)c(C)c4)c3n2)CC1] | 1-(4-(8-((4-((1-ethyl-1H- benzo[d][1,2,3]triazol-5-yl)oxy)-3- methylphenyl)amino)pyrimido[5,4- d]pyrimidin-2-y1)piperazin-1- yl)prop-2-en-1-one | 537.3 (M + H) |
| 134 | 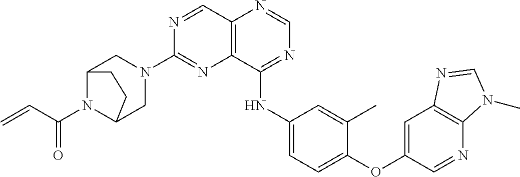 [SMILES: C=CC(=O)N1C2CCC1CN(c1ncc3ncnc(Nc4ccc(Oc5cnc6c(c5)ncn6C)c(C)c4)c3n1)C2] | 1-(3-(8-((3-methyl-4-((3-methyl- 3H-imidazo[4,5-b]pyridin-6- yl)oxy)phenyl)amino)pyrimido[5,4- d]pyrimidin-2-yl)-3,8- diazabicyclo[3.2.1]octan-8-yl)prop- 2-en-1-one | 549.5 (M + H) |
| 135 | 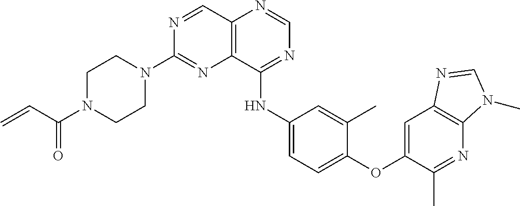 [SMILES: C=CC(=O)N1CCN(c2ncc3ncnc(Nc4ccc(Oc5cc6ncn(C)c6nc5C)c(C)c4)c3n2)CC1] | 1-(4-(8-((4-((3,5-dimethyl-3H- imidazo[4,5-b]pyridin-6-yl)oxy)-3- methylphenyl)amino)pyrimido[5,4- d]pyrimidin-2-yl)piperazin-1- yl)prop-2-en-1-one | 537.3 (M + H) |
| 136 | 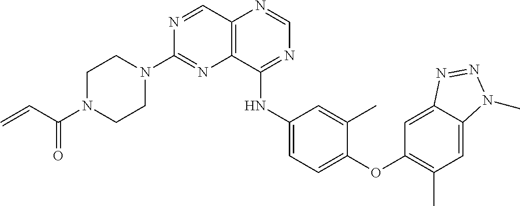 [SMILES: C=CC(=O)N1CCN(c2ncc3ncnc(Nc4ccc(Oc5cc6nnn(C)c6cc5C)c(C)c4)c3n2)CC1] | 1-(4-(8-((4-((1,6-dimethyl-1H- benzo[d][1,2,3]triazol-5-y1)oxy)-3- methylphenyl)amino)pyrimido[5,4- d]pyrimidin-2-yl)piperazin-1- yl)prop-2-en-1-one | 537.2 (M + H) |
| 137 | 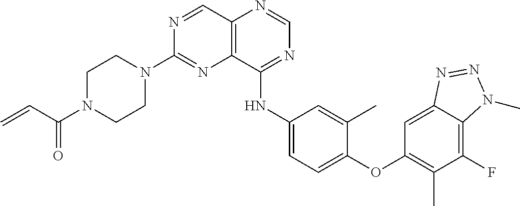 [SMILES: C=CC(=O)N1CCN(c2ncc3ncnc(Nc4ccc(Oc5cc6nnn(C)c6c(F)c5C)c(C)c4)c3n2)CC1] | 1-(4-(8-((4-((7-fluoro-1,6-dimethyl- 1H-benzo[d][1,2,3]triazol-5- yl)oxy)-3- methylphenyl)amino)pyrimido[5,4- d]pyrimidin-2-yl)piperazin-1- yl)prop-2-en-1-one | 555.2 (M + H) |
| 138 | 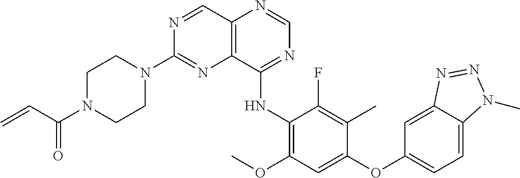 [SMILES: C=CC(=O)N1CCN(c2ncc3ncnc(Nc4c(OC)cc(Oc5ccc6c(c5)nnn6C)c(C)c4F)c3n2)CC1] | 1-(4-(8-((2-fluoro-6-methoxy-3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 571.3 (M + H) |
| 139 | 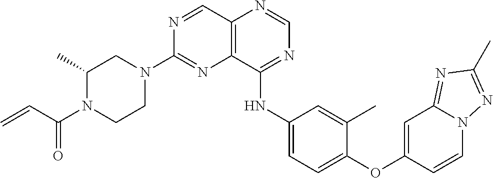 [SMILES: C=CC(=O)N1CCN(c2ncc3ncnc(Nc4ccc(Oc5ccn6nc(C)nc6c5)c(C)c4)c3n2)C[C@H]1C] | (R)-1-(2-methyl-4-(8-((3-methyl-4- ((2-methyl-[1,2,4]triazolo[1,5- alpyridin-7- yl)oxy)phenyl)amino)pyrimido[5,4- d]pyrimidin-2-yl)piperazin-1- yl)prop-2-en-1-one | 537.3 (M + H) |
| 140 | 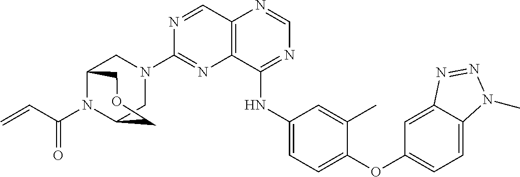 [SMILES: C=CC(=O)N1[C@@H]2COC[C@H]1CN(c1ncc3ncnc(Nc4ccc(Oc5ccc6c(c5)nnn6C)c(C)c4)c3n1)C2] | 1-((1R,5S)-7-(8-((3-methyl-4-((1- methyl-1H-benzo[d][1,2,3]triazol- 5-yl)oxy)phenyl)amino)pyrimido [5,4-d]pyrimidin-2-yl)-3-oxa-7,9- diazabicyclo[3.3.1]nonan-9- yl)prop-2-en-1-one | 565.3 (M + H) |
| 141 | 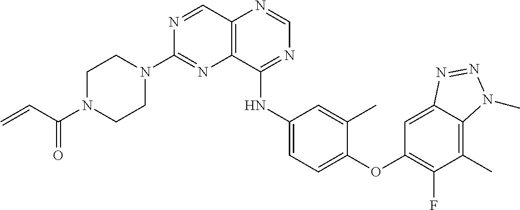 [SMILES: C=CC(=O)N1CCN(c2ncc3ncnc(Nc4ccc(Oc5cc6nnn(C)c6c(C)c5F)c(C)c4)c3n2)CC1] | 1-(4-(8-((4-((6-fluoro-1,7-dimethyl- 1H-benzo[d][1,2,3]triazol-5- yl)oxy)-3- methylphenyl)amino)pyrimido[5,4- d]pyrimidin-2-yl)piperazin-1- yl)prop-2-en-1-one | 555.3 (M + H) |
| 142 | 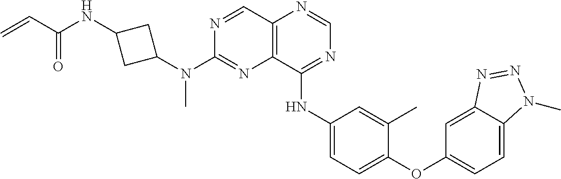 [SMILES: C=CC(=O)NC1CC(N(C)c2ncc3ncnc(Nc4ccc(Oc5ccc6c(c5)nnn6C)c(C)c4)c3n2)C1] | N-(3-(methyl(8-((3-methyl-4-((1- methyl-1H-benzo[d][1,2,3]triazol- 5-yl)oxy)phenyl)amino)pyrimido [5,4-d]pyrimidin-2- yl)amino)cyclobutyl)acrylamide | 537.2 (M + H) |
| 143 | 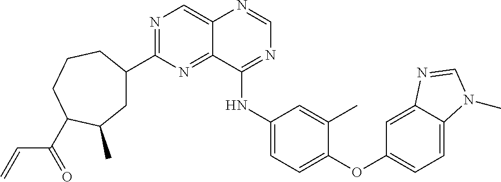 [SMILES: C=CC(=O)C1CCCC(c2ncc3ncnc(Nc4ccc(Oc5ccc6c(c5)ncn6C)c(C)c4)c3n2)C[C@H]1C] | (R)-1-(2-methyl-4-(8-((3-methyl-4- ((1-methyl-1H-benzo[d]imidazol-5- yl)oxy)phenyl)amino)pyrimido[5,4- d]pyrimidin-2-yl)-1,4-diazepan-1- yl)prop-2-en-1-one | 550.3 (M + H) |
| 144 | 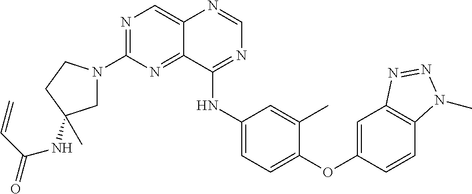 [SMILES: C=CC(=O)N[C@]1(C)CCN(c2ncc3ncnc(Nc4ccc(Oc5ccc6c(c5)nnn6C)c(C)c4)c3n2)C1] | (R)-N-(3-methyl-1-(8-((3-methyl-4- ((1-methyl-1H- benzo[d][1,2,3]triazol-5- yl)oxy)phenyl)amino)pyrimido[5,4- d]pyrimidin-2-yl)pyrrolidin-3- yl)acrylamide | 537.3 (M + H) |
| 145 | 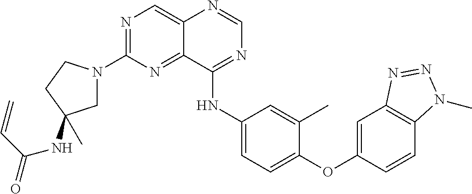 [SMILES: C=CC(=O)N[C@@]1(C)CCN(c2ncc3ncnc(Nc4ccc(Oc5ccc6c(c5)nnn6C)c(C)c4)c3n2)C1] | (S)-N-(3-methyl-1-(8-((3-methyl-4- ((1-methyl-1H- benzo[d][1,2,3]triazol-5- yl)oxy)phenyl)amino)pyrimido[5,4- d]pyrimidin-2-yl)pyrrolidin-3- yl)acrylamide | 537.4 (M + H) |
| 146 | 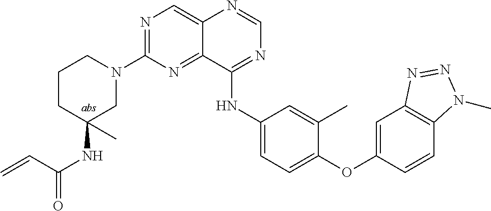 [SMILES: C=CC(=O)N[C@@]1(C)CCCN(c2ncc3ncnc(Nc4ccc(Oc5ccc6c(c5)nnn6C)c(C)c4)c3n2)C1] | (S)-N-(3-methyl-1-(8-((3-methyl-4- ((1-methyl-1H- benzo[d][1,2,3]triazol-5- yl)oxy)phenyl)amino)pyrimido[5,4- d]pyrimidin-2-yl)piperidin-3- yl)acrylamide | 551.3 (M + H) |
| 147 | 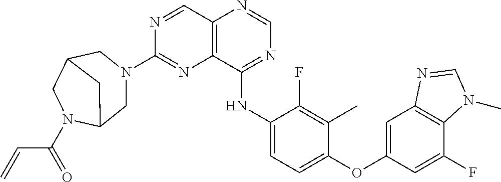 [SMILES: C=CC(=O)N1CC2CC1CN(c1ncc3ncnc(Nc4ccc(Oc5cc(F)c6c(c5)ncn6C)c(C)c4F)c3n1)C2] | 1-(3-(8-((2-fluoro-4-((7-fluoro-1- methyl-1H-benzo[d]imidazol-5- yl)oxy)-3- methylphenyl)amino)pyrimido[5,4- d]pyrimidin-2-y1)-3,6- diazabicyclo[3.2.1]octan-6-yl)prop- 2-en-1-one | 584.4 (M + H) |
| 148 | 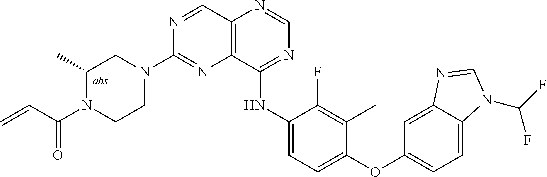 [SMILES: C=CC(=O)N1CCN(c2ncc3ncnc(Nc4ccc(Oc5ccc6c(c5)ncn6C(F)F)c(C)c4F)c3n2)C[C@H]1C] | (R)-1-(4-(8-((4-((1- (difluoromethyl)-1H- benzo[d]imidazol-5-yl)oxy)-2- fluoro-3- methylphenyl)amino)pyrimido[5,4- d]pyrimidin-2-yl)-2- methylpiperazin-1-yl)prop-2-en-1- one | 590.3 (M + H) |
| 149 | 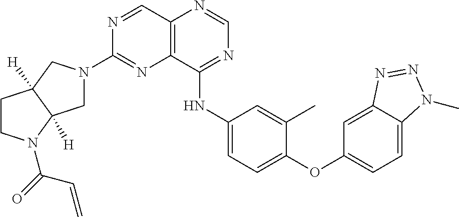 [SMILES: C=CC(=O)N1CC[C@H]2CN(c3ncc4ncnc(Nc5ccc(Oc6ccc7c(c6)nnn7C)c(C)c5)c4n3)C[C@H]21] | 1-((3aS,6aS)-5-(8-((3-methyl-4-((1- methyl-1H-benzo[d][1,2,3]triazol- 5-yl)oxy)phenyl)amino)pyrimido [5,4-d]pyrimidin-2- yl)hexahydropyrrolo[3,4-b]pyrrol- 1(2H)-y1)prop-2-en-1-one | 549.3 (M + H) |
| 150 | 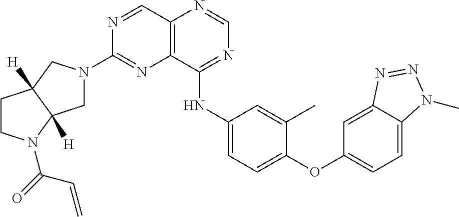 [SMILES: C=CC(=O)N1CC[C@@H]2CN(c3ncc4ncnc(Nc5ccc(Oc6ccc7c(c6)nnn7C)c(C)c5)c4n3)C[C@@H]21] | 1-((3aR,6aR)-5-(8-((3-methyl-4- ((1-methyl-1H- benzo[d][1,2,3]triazol-5- yl)oxy)phenyl)amino)pyrimido[5,4- d]pyrimidin-2- yl)hexahydropyrrolo[3,4-b]pyrrol- 1(2H)-yl)prop-2-en-1-one | 549.2 (M + H) |
| 151 | 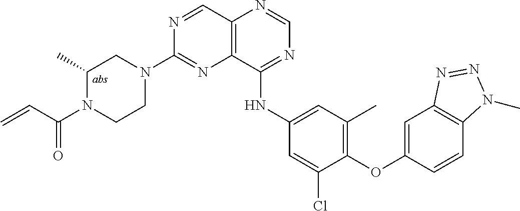 [SMILES: C=CC(=O)N1CCN(c2ncc3ncnc(Nc4cc(C)c(Oc5ccc6c(c5)nnn6C)c(Cl)c4)c3n2)C[C@H]1C] | (R)-1-(4-(8-((3-chloro-5-methyl-4- ((1-methyl-1H- benzo[d][1,2,3]triazol-5- yl)oxy)phenyl)amino)pyrimido[5,4- d]pyrimidin-2-yl)-2- methylpiperazin-1-yl)prop-2-en-1- one | 571.2 (M + H) |
| 152 | 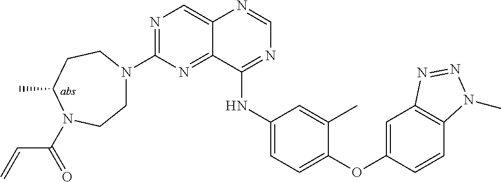 [SMILES: C=CC(=O)N1CCN(c2ncc3ncnc(Nc4ccc(Oc5ccc6c(c5)nnn6C)c(C)c4)c3n2)CC[C@H]1C] | (R)-1-(7-methyl-4-(8-((3-methyl-4- ((1-methyl-1H- benzo[d][1,2,3]triazol-5- yl)oxy)phenyl)amino)pyrimido[5,4- d]pyrimidin-2-y1)-1,4-diazepan-1- yl)prop-2-en-1-one | 551.2 (M + H) |
| 153 | 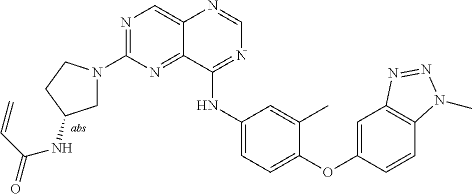 [SMILES: C=CC(=O)N[C@@H]1CCN(c2ncc3ncnc(Nc4ccc(Oc5ccc6c(c5)nnn6C)c(C)c4)c3n2)C1] | (R)-N-(1-(8-((3-methyl-4-((1- methyl-1H-benzo[d][1,2,3]triazol- 5-yl)oxy)phenyl)amino)pyrimido [5,4-d]pyrimidin-2-yl)pyrrolidin-3- yl)acrylamide | 523.2 (M + H) |
| 154 | 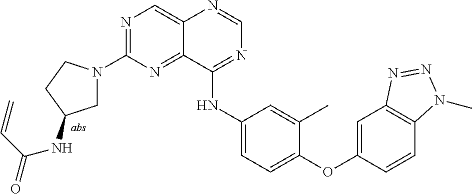 [SMILES: C=CC(=O)N[C@H]1CCN(c2ncc3ncnc(Nc4ccc(Oc5ccc6c(c5)nnn6C)c(C)c4)c3n2)C1] | (S)-N-(1-(8-((3-methyl-4-((1- methyl-1H-benzo[d][1,2,3]triazol- 5-yl)oxy)phenyl)amino)pyrimido [5,4- d]pyrimidin-2-yl)pyrrolidin-3- yl)acrylamide | 523.2 (M + H) |
| 155 | 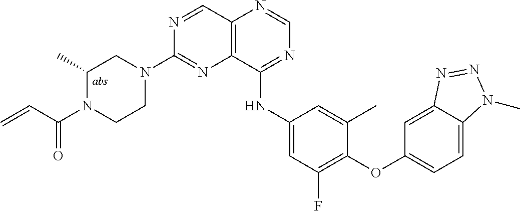 [SMILES: C=CC(=O)N1CCN(c2ncc3ncnc(Nc4cc(C)c(Oc5ccc6c(c5)nnn6C)c(F)c4)c3n2)C[C@H]1C] | (R)-1-(4-(8-((3-fluoro-5-methyl-4- ((1-methyl-1H- benzo[d][1,2,3]triazol-5- yl)oxy)phenyl)amino)pyrimido[5,4- d]pyrimidin-2-y1)-2- methylpiperazin-1-yl)prop-2-en-1- one | 555.3 (M + H) |
| 156 | 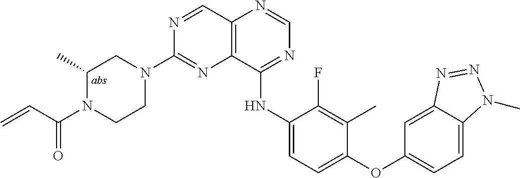 [SMILES: C=CC(=O)N1CCN(c2ncc3ncnc(Nc4ccc(Oc5ccc6c(c5)nnn6C)c(C)c4F)c3n2)C[C@H]1C] | (R)-1-(4-(8-((2-fluoro-3-methyl-4- ((1-methyl-1H- benzo[d][1,2,3]triazol-5- yl)oxy)phenyl)amino)pyrimido[5,4- d]pyrimidin-2-y1)-2- methylpiperazin-1-yl)prop-2-en-1- one | 555.3 (M + H) |
| 157 | 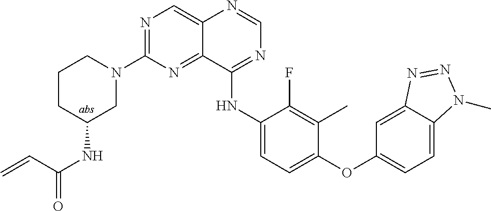 [SMILES: C=CC(=O)N[C@@H]1CCCN(c2ncc3ncnc(Nc4ccc(Oc5ccc6c(c5)nnn6C)c(C)c4F)c3n2)C1] | (R)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)piperidin-3- yl)acrylamide | 555.3 (M + H) |
| 158 | 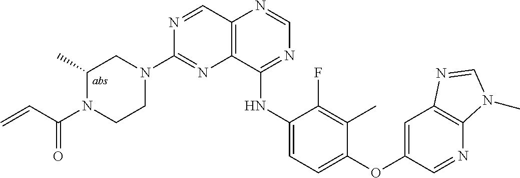 [SMILES: C=CC(=O)N1CCN(c2ncc3ncnc(Nc4ccc(Oc5cnc6c(c5)ncn6C)c(C)c4F)c3n2)C[C@H]1C] | (R)-1-(4-(8-((2-fluoro-3-methyl-4- ((3-methyl-3H-imidazo[4,5- b]pyridin-6- yl)oxy)phenyl)amino)pyrimido[5,4- d]pyrimidin-2-y1)-2- methylpiperazin-1-yl)prop-2-en-1- one | 555.1 (M + H) |
| 159 | 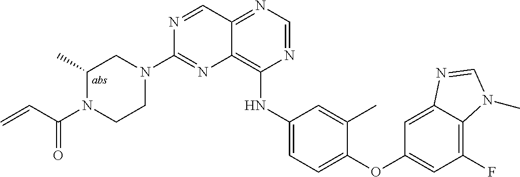 [SMILES: C=CC(=O)N1CCN(c2ncc3ncnc(Nc4ccc(Oc5cc(F)c6c(c5)ncn6C)c(C)c4)c3n2)C[C@H]1C] | (R)-1-(4-(8-((4-((7-fluoro-1- methyl-1H-benzo[d]imidazol-5- yl)oxy)-3- methylphenyl)amino)pyrimido[5,4- d]pyrimidin-2-yl)-2- methylpiperazin-1-yl)prop-2-en-1- one | 554.2 (M + H) |
| 160 | 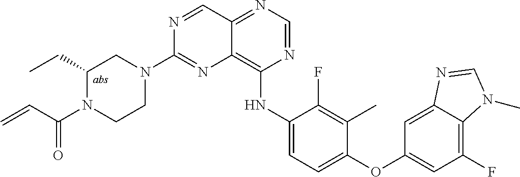 [SMILES: C=CC(=O)N1CCN(c2ncc3ncnc(Nc4ccc(Oc5cc(F)c6c(c5)ncn6C)c(C)c4F)c3n2)C[C@H]1CC] | (R)-1-(2-ethyl-4-(8-((2-fluoro-4- ((7-fluoro-1-methyl-1H- benzo[d]imidazol-5-yl)oxy)-3- methylphenyl)amino)pyrimido[5,4- d]pyrimidin-2-yl)piperazin-1- yl)prop-2-en-1-one | 586.3 (M + H) |
| 161 | 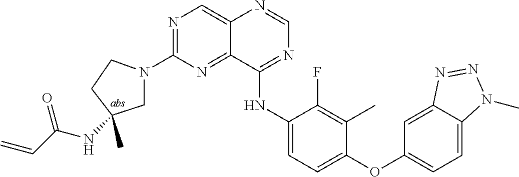 [SMILES: C=CC(=O)N[C@]1(C)CCN(c2ncc3ncnc(Nc4ccc(Oc5ccc6c(c5)nnn6C)c(C)c4F)c3n2)C1] | (R)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)-3- methylpyrrolidin-3-yl)acrylamide | 555.3 (M + H) |
| 162 | 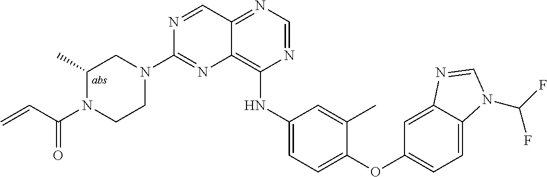 [SMILES: C=CC(=O)N1CCN(c2ncc3ncnc(Nc4ccc(Oc5ccc6c(c5)ncn6C(F)F)c(C)c4)c3n2)C[C@H]1C] | (R)-1-(4-(8-((4-((1- (difluoromethyl)-1H- benzo[d]imidazol-5-yl)oxy)-3- methylphenyl)amino)pyrimido[5,4- d]pyrimidin-2-y1)-2- methylpiperazin-1-yl)prop-2-en-1- one | 572.4 (M + H) |
| 163 | 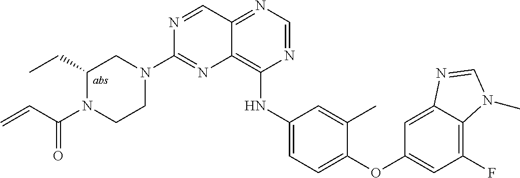 [SMILES: C=CC(=O)N1CCN(c2ncc3ncnc(Nc4ccc(Oc5cc(F)c6c(c5)ncn6C)c(C)c4)c3n2)C[C@H]1CC] | (R)-1-(2-ethyl-4-(8-((4-((7-fluoro- 1-methyl-1H-benzo[d]imidazol-5- yl)oxy)-3- methylphenyl)amino)pyrimido[5,4- d]pyrimidin-2-yl)piperazin-1- yl)prop-2-en-1-one | 568.3 (M + H) |
| 164 | 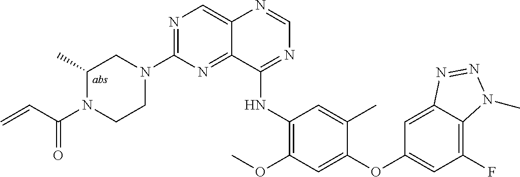 [SMILES: C=CC(=O)N1CCN(c2ncc3ncnc(Nc4cc(C)c(Oc5cc(F)c6c(c5)nnn6C)cc4OC)c3n2)C[C@H]1C] | (R)-1-(4-(8-((4-((7-fluoro-1- methyl-1H-benzo[d][1,2,3]triazol- 5-yl)oxy)-2-methoxy-5- methylphenyl)amino)pyrimido[5,4- d]pyrimidin-2-y1)-2- methylpiperazin-1-yl)prop-2-en-1- one | 585.2 (M + H) |
| 165 | 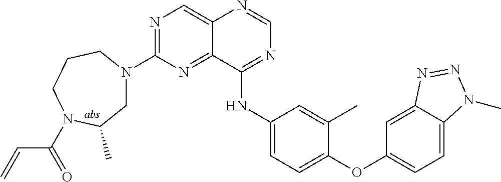 [SMILES: C=CC(=O)N1CCCN(c2ncc3ncnc(Nc4ccc(Oc5ccc6c(c5)nnn6C)c(C)c4)c3n2)C[C@@H]1C] | (S)-1-(2-methyl-4-(8-((3-methyl-4- ((1-methyl-1H- benzo[d][1,2,3]triazol-5- yl)oxy)phenyl)amino)pyrimido[5,4- d]pyrimidin-2-y1)-1,4-diazepan-1- yl)prop-2-en-1-one | 551.2 (M + H) |
| 166 | 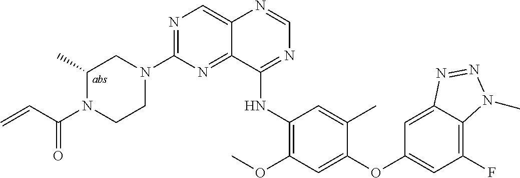 [SMILES: C=CC(=O)N1CCN(c2ncc3ncnc(Nc4cc(C)c(Oc5cc(F)c6c(c5)nnn6C)cc4OC)c3n2)C[C@H]1C] | (R)-1-(4-(8-((4-((7-fluoro-1- methyl-1H-benzo[d][1,2,3]triazol- 5-yl)oxy)-2-methoxy-5- methylphenyl)amino)pyrimido[5,4- d]pyrimidin-2-y1)-2- methylpiperazin-1-yl)prop-2-en-1- one | 585.2 (M + H) |
| 167 | 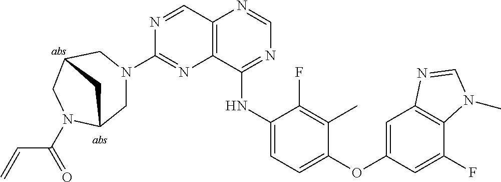 [SMILES: C=CC(=O)N1C[C@H]2C[C@@H]1CN(c1ncc3ncnc(Nc4ccc(Oc5cc(F)c6c(c5)ncn6C)c(C)c4F)c3n1)C2] | 1-((1S,5R)-3-(8-((2-fluoro-4-((7- fluoro-1-methyl-1H- benzo[d]imidazol-5-y1)oxy)-3- methylphenyl)amino)pyrimido[5,4- d]pyrimidin-2-yl)-3,6- diazabicyclo[3.2.1]octan-6-yl)prop- 2-en-1-one | 584.2 (M + H) |
| 168 | 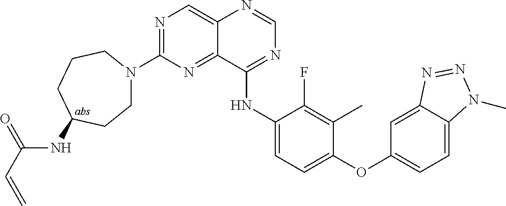 [SMILES: C=CC(=O)N[C@H]1CCCN(c2ncc3ncnc(Nc4ccc(Oc5ccc6c(c5)nnn6C)c(C)c4F)c3n2)CC1] | (S)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)azepan-4- yl)acrylamide | 569.2 (M + H) |
| 169 | 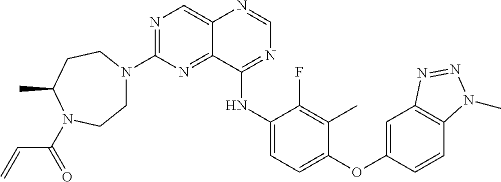 [SMILES: C=CC(=O)N1CCN(c2ncc3ncnc(Nc4ccc(Oc5ccc6c(c5)nnn6C)c(C)c4F)c3n2)CC[C@@H]1C] | (S)-1-(4-(8-((2-fluoro-3-methyl-4- ((1-methyl-1H- benzo[d][1,2,3]triazol-5- yl)oxy)phenyl)amino)pyrimido[5,4- d]pyrimidin-2-y1)-7-methyl-1,4- diazepan-1-yl)prop-2-en-1-one | 569.2 (M + H) |
| 170 | 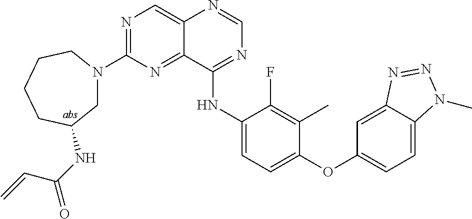 [SMILES: C=CC(=O)N[C@@H]1CCCCN(c2ncc3ncnc(Nc4ccc(Oc5ccc6c(c5)nnn6C)c(C)c4F)c3n2)C1] | (R)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)azepan-3- yl)acrylamide | 569.2 (M + H) |
| 171 | 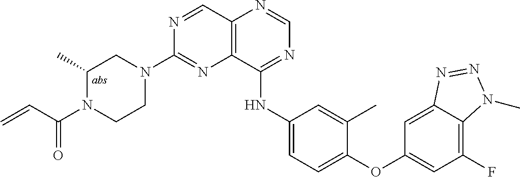 [SMILES: C=CC(=O)N1CCN(c2ncc3ncnc(Nc4ccc(Oc5cc(F)c6c(c5)nnn6C)c(C)c4)c3n2)C[C@H]1C] | (R)-1-(4-(8-((4-((7-fluoro-1- methyl-1H-benzo[d][1,2,3]triazol- 5-yl)oxy)-3- methylphenyl)amino)pyrimido[5,4- d]pyrimidin-2-y1)-2- methylpiperazin-1-yl)prop-2-en-1- one | 555.2 (M + H) |
| 172 | 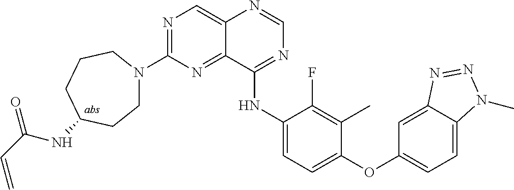 [SMILES: C=CC(=O)N[C@@H]1CCCN(c2ncc3ncnc(Nc4ccc(Oc5ccc6c(c5)nnn6C)c(C)c4F)c3n2)CC1] | (R)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)azepan-4- yl)acrylamide | 569.2 (M + H) |
| 173 | 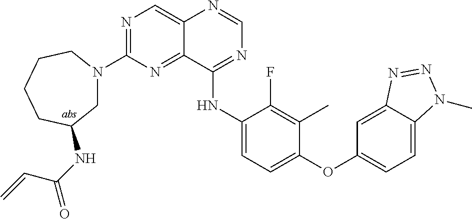 [SMILES: C=CC(=O)N[C@H]1CCCCN(c2ncc3ncnc(Nc4ccc(Oc5ccc6c(c5)nnn6C)c(C)c4F)c3n2)C1] | (S)-N-(1-(8-((2-fluoro-3-methyl-4- ((1-methyl-1H- benzo[d][1,2,3]triazol-5- yl)oxy)phenyl)amino)pyrimido[5,4- d]pyrimidin-2-yl)azepan-3- yl)acrylamide | 569.2 (M + H) |
| 174 | 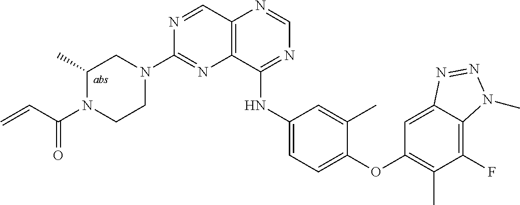 [SMILES: C=CC(=O)N1CCN(c2ncc3ncnc(Nc4ccc(Oc5cc6nnn(C)c6c(F)c5C)c(C)c4)c3n2)C[C@H]1C] | (R)-1-(4-(8-((4-((7-fluoro-1,6- dimethyl-1H- benzo[d][1,2,3]triazol-5-yl)oxy)-3- methylphenyl)amino)pyrimido[5,4- d]pyrimidin-2-yl)-2- methylpiperazin-1-yl)prop-2-en-1- one | 569.2 (M + H) |
| 175 | 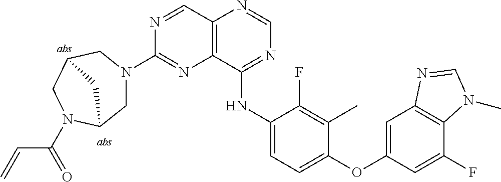 [SMILES: C=CC(=O)N1C[C@@H]2C[C@H]1CN(c1ncc3ncnc(Nc4ccc(Oc5cc(F)c6c(c5)ncn6C)c(C)c4F)c3n1)C2] | 1-((1R,5S)-3-(8-((2-fluoro-4-((7- fluoro-1-methyl-1H- benzo[d]imidazol-5-y1)oxy)-3- methylphenyl)amino)pyrimido[5,4- d]pyrimidin-2-yl)-3,6- diazabicyclo[3.2.1]octan-6-yl)prop- 2-en-1-one | 584.2 (M + H) |
| 176 | 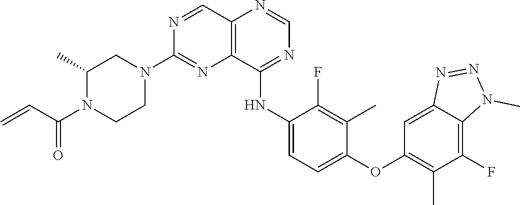 [SMILES: C=CC(=O)N1CCN(c2ncc3ncnc(Nc4ccc(Oc5cc6nnn(C)c6c(F)c5C)c(C)c4F)c3n2)C[C@H]1C] | (R)-1-(4-(8-((2-fluoro-4-((7-fluoro- 1,6-dimethyl-1H- benzo[d][1,2,3]triazol-5-yl)oxy)-3- methylphenyl)amino)pyrimido[5,4- d]pyrimidin-2-yl)-2- methylpiperazin-1-yl)prop-2-en-1- one | 587.2 (M + H) |
| 177 | 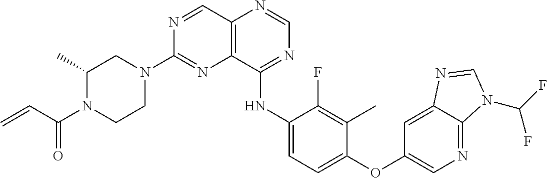 [SMILES: C=CC(=O)N1CCN(c2ncc3ncnc(Nc4ccc(Oc5cnc6c(c5)ncn6C(F)F)c(C)c4F)c3n2)C[C@H]1C] | (R)-1-(4-(8-((4-((3- (difluoromethyl)-3H-imidazo[4,5- b]pyridin-6-yl)oxy)-2-fluoro-3- methylphenyl)amino)pyrimido[5,4- d]pyrimidin-2-y1)-2- methylpiperazin-1-yl)prop-2-en-1- one | 591.2 (M + H) |
| 178 | 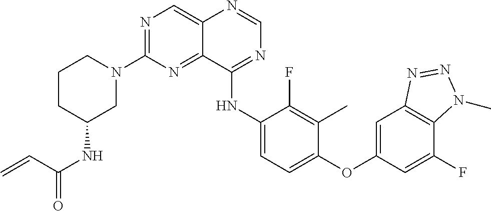 [SMILES: C=CC(=O)N[C@@H]1CCCN(c2ncc3ncnc(Nc4ccc(Oc5cc(F)c6c(c5)nnn6C)c(C)c4F)c3n2)C1] | (R)-N-(1-(8-((2-fluoro-4-((7-fluoro- 1-methyl-1H- benzo[d][1,2,3]triazol-5-yl)oxy)-3- methylphenyl)amino)pyrimido[5,4- d]pyrimidin-2-yl)piperidin-3- yl)acrylamide | 573.1 (M + H) |
| 179 | 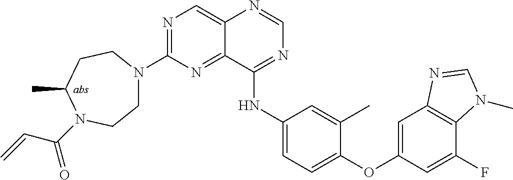 [SMILES: C=CC(=O)N1CCN(c2ncc3ncnc(Nc4ccc(Oc5cc(F)c6c(c5)ncn6C)c(C)c4)c3n2)CC[C@@H]1C] | (S)-1-(4-(8-((4-((7-fluoro-1-methyl- 1H-benzo[d]imidazol-5-yl)oxy)-3- methylphenyl)amino)pyrimido[5,4- d]pyrimidin-2-y1)-7-methyl-1,4- diazepan-1-yl)prop-2-en-1-one | 568.1 (M + H) |
| 180 | 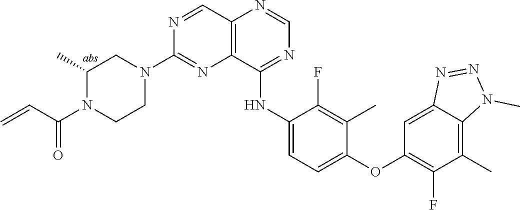 [SMILES: C=CC(=O)N1CCN(c2ncc3ncnc(Nc4ccc(Oc5cc6nnn(C)c6c(C)c5F)c(C)c4F)c3n2)C[C@H]1C] | (R)-1-(4-(8-((2-fluoro-4-((6-fluoro- 1,7-dimethyl-1H- benzo[d][1,2,3]triazol-5-yl)oxy)-3- methylphenyl)amino)pyrimido[5,4- d]pyrimidin-2-y1)-2- methylpiperazin-1-yl)prop-2-en-1- one | 587.0 (M + H) |
| 181 | 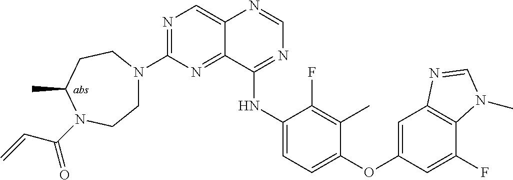 [SMILES: C=CC(=O)N1CCN(c2ncc3ncnc(Nc4ccc(Oc5cc(F)c6c(c5)ncn6C)c(C)c4F)c3n2)CC[C@@H]1C] | (S)-1-(4-(8-((2-fluoro-4-((7-fluoro- 1-methyl-1H-benzo[d]imidazol-5- yl)oxy)-3- methylphenyl)amino)pyrimido[5,4- d]pyrimidin-2-y1)-7-methyl-1,4- diazepan-1-yl)prop-2-en-1-one | 586.4 (M + H) |
| 182 | 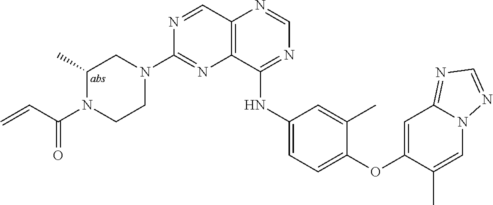 [SMILES: C=CC(=O)N1CCN(c2ncc3ncnc(Nc4ccc(Oc5cc6ncnn6cc5C)c(C)c4)c3n2)C[C@H]1C] | (R)-1-(2-methyl-4-(8-((3-methyl-4- ((6-methyl-[1,2,4]triazolo[1,5- alpyridin-7- yl)oxy)phenyl)amino)pyrimido[5,4- d]pyrimidin-2-yl)piperazin-1- yl)prop-2-en-1-one | 537.2 (M + H) |
| 183 | 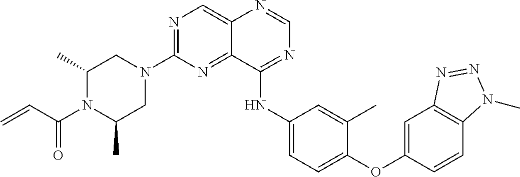 [SMILES: C=CC(=O)N1[C@H](C)CN(c2ncc3ncnc(Nc4ccc(Oc5ccc6c(c5)nnn6C)c(C)c4)c3n2)C[C@H]1C] | 1-((2R,6R)-2,6-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 184 | 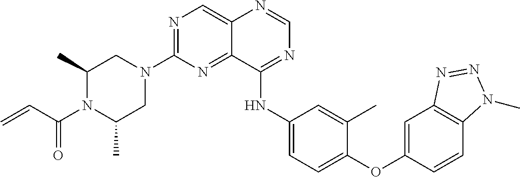 [SMILES: C=CC(=O)N1[C@@H](C)CN(c2ncc3ncnc(Nc4ccc(Oc5ccc6c(c5)nnn6C)c(C)c4)c3n2)C[C@@H]1C] | 1-((2S,6S)-2,6-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 185 | 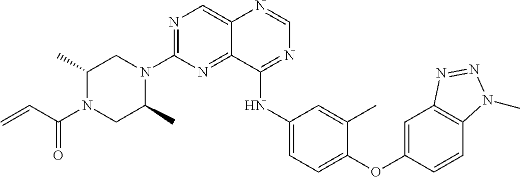 [SMILES: C=CC(=O)N1C[C@H](C)N(c2ncc3ncnc(Nc4ccc(Oc5ccc6c(c5)nnn6C)c(C)c4)c3n2)C[C@H]1C] | 1-((2S,6S)-2,6-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 186 | 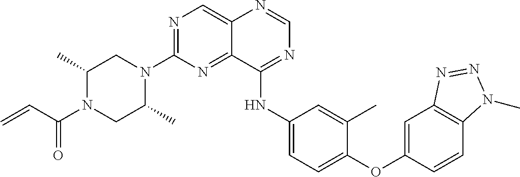 [SMILES: C=CC(=O)N1C[C@@H](C)N(c2ncc3ncnc(Nc4ccc(Oc5ccc6c(c5)nnn6C)c(C)c4)c3n2)C[C@H]1C] | 1-((2R,5R)-2,5-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 187 | 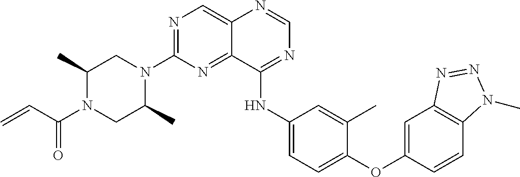 [SMILES: C=CC(=O)N1C[C@H](C)N(c2ncc3ncnc(Nc4ccc(Oc5ccc6c(c5)nnn6C)c(C)c4)c3n2)C[C@@H]1C] | 1-((2S,5S)-2,5-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 188 | 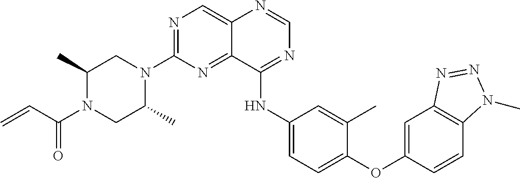 [SMILES: C=CC(=O)N1C[C@@H](C)N(c2ncc3ncnc(Nc4ccc(Oc5ccc6c(c5)nnn6C)c(C)c4)c3n2)C[C@@H]1C] | 1-((2S,5R)-2,5-dimethyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 551.3 (M + H) |
| 189 | 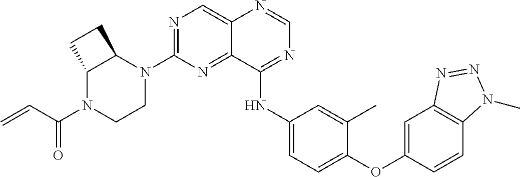 [SMILES: C=CC(=O)N1CCN(c2ncc3ncnc(Nc4ccc(Oc5ccc6c(c5)nnn6C)c(C)c4)c3n2)[C@@H]2CC[C@H]21] | 1-((1R,6R)-5-(8-((3-methyl-4-((1- methyl-1H-benzo[d][1,2,3]triazol- 5- yl)oxy)phenyl)amino)pyrimido[5,4- d]pyrimidin-2-y1)-2,5- diazabicyclo[4.2.0]octan-2-yl)prop- 2-en-1-one | 549.3 (M + H) |
| 190 | 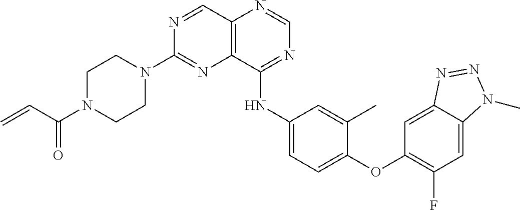 [SMILES: C=CC(=O)N1CCN(c2ncc3ncnc(Nc4ccc(Oc5cc6nnn(C)c6cc5F)c(C)c4)c3n2)CC1] | 1-(4-(8-((4-((6-fluoro-1-methyl-1H- benzo[d][1,2,3]triazol-5-yl)oxy)-3- methylphenyl)amino)pyrimido[5,4- d]pyrimidin-2-yl)piperazin-1- yl)prop-2-en-1-one | 541.5 (M + H) |
| 191 | 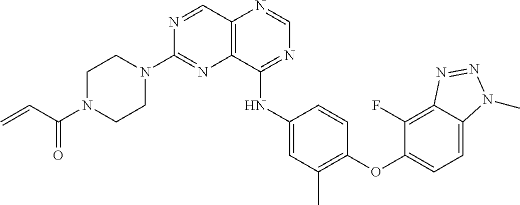 [SMILES: C=CC(=O)N1CCN(c2ncc3ncnc(Nc4ccc(Oc5ccc6c(nnn6C)c5F)c(C)c4)c3n2)CC1] | 1-(4-(8-((4-((4-fluoro-1-methyl-1H- benzo[d][1,2,3]triazol-5-yl)oxy)-3- methylphenyl)amino)pyrimido[5,4- d]pyrimidin-2-yl)piperazin-1- yl)prop-2-en-1-one | 541.2 (M + H) |
| 192 | 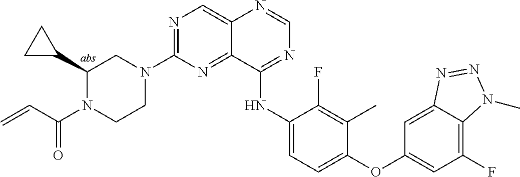 [SMILES: C=CC(=O)N1CCN(c2ncc3ncnc(Nc4ccc(Oc5cc(F)c6c(c5)nnn6C)c(C)c4F)c3n2)C[C@@H]1C1CC1] | (S)-1-(2-cyclopropyl-4-(8-((2- fluoro-4-((7-fluoro-1-methyl-1H- benzo[d][1,2,3]triazol-5-yl)oxy)-3- methylphenyl)amino)pyrimido[5,4- d]pyrimidin-2-y1)piperazin-1- yl)prop-2-en-1-one | 599.2 (M + H) |
| 193 | 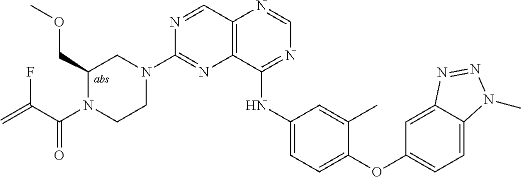 [SMILES: C=C(F)C(=O)N1CCN(c2ncc3ncnc(Nc4ccc(Oc5ccc6c(c5)nnn6C)c(C)c4)c3n2)C[C@@H]1COC] | (R)-2-fluoro-1-(2- (methoxymethyl)-4-(8-((3-methyl- 4-((1-methyl-1H- benzo[d][1,2,3]triazol-5- yl)oxy)phenyl)amino)pyrimido[5,4- d]pyrimidin-2-yl)piperazin-1- yl)prop-2-en-1-one | 585.4 (M + H) |
| 194 | 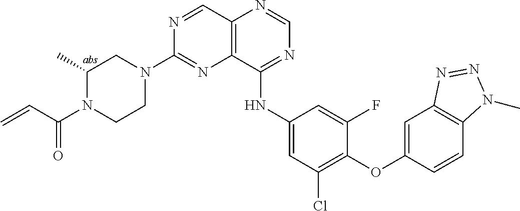 [SMILES: C=CC(=O)N1CCN(c2ncc3ncnc(Nc4cc(F)c(Oc5ccc6c(c5)nnn6C)c(Cl)c4)c3n2)C[C@H]1C] | (R)-1-(4-(8-((3-chloro-5-fluoro-4- ((1-methyl-1H- benzo[d][1,2,3]triazol-5- yl)oxy)phenyl)amino)pyrimido[5,4- d]pyrimidin-2-y1)-2- methylpiperazin-1-yl)prop-2-en-1- one | 575.3 (M + H) |
| 195 | 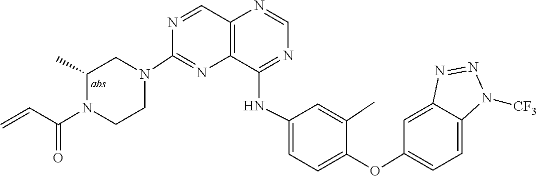 [SMILES: C=CC(=O)N1CCN(c2ncc3ncnc(Nc4ccc(Oc5ccc6c(c5)nnn6C(F)(F)F)c(C)c4)c3n2)C[C@H]1C] | (R)-1-(2-methyl-4-(8-((3-methyl-4- ((1-(trifluoromethyl)-1H- benzo[d][1,2,3]triazol-5- yl)oxy)phenyl)amino)pyrimido[5,4- d]pyrimidin-2-yl)piperazin-1- yl)prop-2-en-1-one | 591.3 (M + H) |

Compounds 196 to 208 in Table 16 below were prepared as a racemic mixture according to Example 55 Steps A to C. The enantiomers were separated using chiral supercritical fluid chromatography (SFC).

TABLE 16
Compounds 193-205

| | | | MS |
|---|---|---|---|
| Cmp. | | | API |
| ID | Structure | Chemical Name | (m/z) |
| 196 | 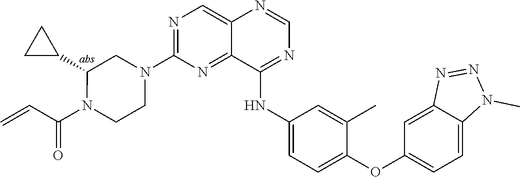 [SMILES: C=CC(=O)N1CCN(c2ncc3ncnc(Nc4ccc(Oc5ccc6c(c5)nnn6C)c(C)c4)c3n2)C[C@H]1C1CC1] | (R)-1-(2-cyclopropyl-4-(8-((3- methyl-4-((1-methyl-1H- benzo[d][1,2,3]triazol-5- yl)oxy)phenyl)amino)pyrimido[5,4- d]pyrimidin-2-yl)piperazin-1-yl)prop- 2-en-1-one | 563.4 (M + H) |
| 197 | 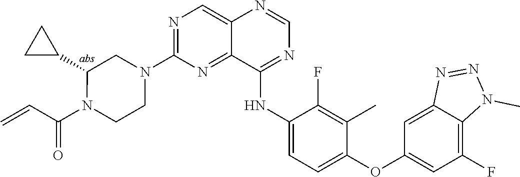 [SMILES: C=CC(=O)N1CCN(c2ncc3ncnc(Nc4ccc(Oc5cc(F)c6c(c5)nnn6C)c(C)c4F)c3n2)C[C@H]1C1CC1] | (R)-1-(2-cyclopropyl-4-(8-((2-fluoro- 4-((7-fluoro-1-methyl-1H- benzo[d][1,2,3]triazol-5-yl)oxy)-3- methylphenyl)amino)pyrimido[5,4- d]pyrimidin-2-yl)piperazin-1-yl)prop- 2-en-1-one | 599.2 (M + H) |
| 198 | 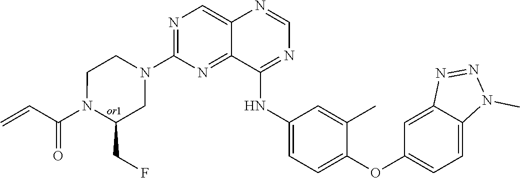 [SMILES: C=CC(=O)N1CCN(c2ncc3ncnc(Nc4ccc(Oc5ccc6c(c5)nnn6C)c(C)c4)c3n2)C[C@H]1CF] | (S)-1-(2-(fluoromethyl)-4-(8-((3- methyl-4-((1-methyl-1H- benzo[d][1,2,3]triazol-5- yl)oxy)phenyl)amino)pyrimido[5,4- d]pyrimidin-2-yl)piperazin-1-yl)prop- 2-en-1-one | 555.2 (M + H) |
| 199 | 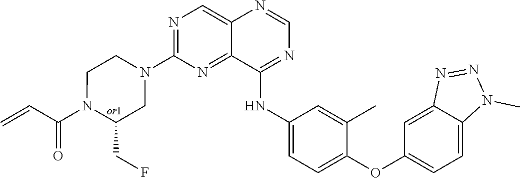 [SMILES: C=CC(=O)N1CCN(c2ncc3ncnc(Nc4ccc(Oc5ccc6c(c5)nnn6C)c(C)c4)c3n2)C[C@@H]1CF] | (R)-1-(2-(fluoromethyl)-4-(8-((3- methyl-4-((1-methyl-1H- benzo[d][1,2,3]triazol-5- yl)oxy)phenyl)amino)pyrimido[5,4- d]pyrimidin-2-yl)piperazin-1-yl)prop- 2-en-1-one | 555.3 (M + H) |
| 200 | 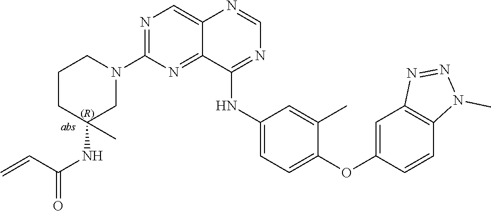 | (R)-N-(3-methyl-1-(8-((3-methyl-4- ((1-methyl-1H- benzo[d][1,2,3]triazol-5- yl)oxy)phenyl)amino)pyrimido[5,4- d]pyrimidin-2-yl)piperidin-3- yl)acrylamide | 551.3 (M + H) |
| 201 | 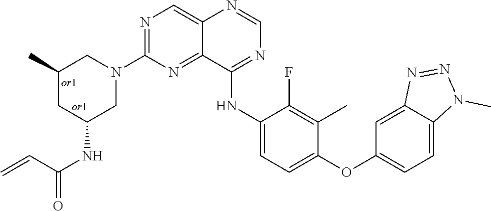 [SMILES: C=CC(=O)N[C@@H]1C[C@@H](C)CN(c2ncc3ncnc(Nc4ccc(Oc5ccc6c(c5)nnn6C)c(C)c4F)c3n2)C1] | N-((3R,5R)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-5-methylpiperidin- 3-yl)acrylamide | 569.3 (M + H) |
| 202 | 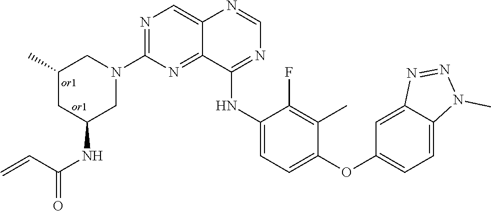 [SMILES: C=CC(=O)N[C@H]1C[C@H](C)CN(c2ncc3ncnc(Nc4ccc(Oc5ccc6c(c5)nnn6C)c(C)c4F)c3n2)C1] | N-((3S,5S)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-5-methylpiperidin- 3-yl)acrylamide | 569.3 (M + H) |
| 203 | 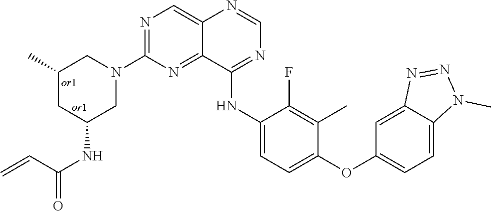 [SMILES: C=CC(=O)N[C@@H]1C[C@H](C)CN(c2ncc3ncnc(Nc4ccc(Oc5ccc6c(c5)nnn6C)c(C)c4F)c3n2)C1] | N-((3R,5S)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-5-methylpiperidin- 3-yl)acrylamide | 569.3 (M + H) |
| 204 | 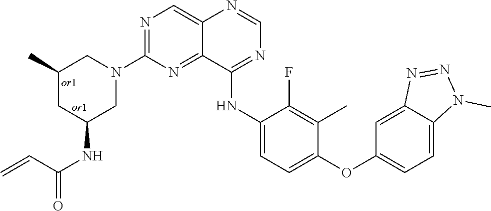 [SMILES: C=CC(=O)N[C@H]1C[C@@H](C)CN(c2ncc3ncnc(Nc4ccc(Oc5ccc6c(c5)nnn6C)c(C)c4F)c3n2)C1] | N-((3S,5R)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-5-methylpiperidin- 3-yl)acrylamide | 569.3 (M + H) |
| 205 | 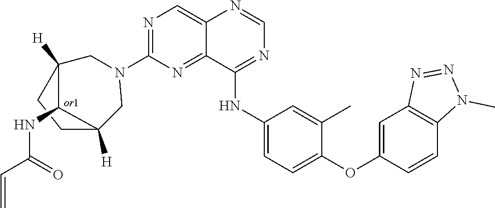 [SMILES: C=CC(=O)N[C@H]1[C@@H]2CC[C@H]1CN(c1ncc3ncnc(Nc4ccc(Oc5ccc6c(c5)nnn6C)c(C)c4)c3n1)C2] | N-((1R,5S,8r)-3-(8-((3-methyl-4-((1- methyl-1H-benzo[d][1,2,3]triazol-5- yl)oxy)phenyl)amino)pyrimido[5,4- d]pyrimidin-2-yl)-3- azabicyclo[3.2.1]octan-8- yl)acrylamide | 563.3 (M + H) |
| 206 | 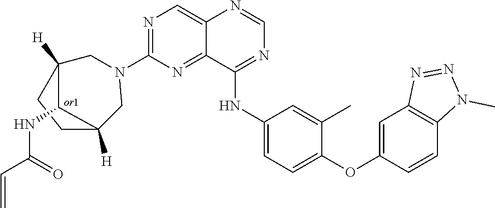 [SMILES: C=CC(=O)N[C@@H]1[C@@H]2CC[C@H]1CN(c1ncc3ncnc(Nc4ccc(Oc5ccc6c(c5)nnn6C)c(C)c4)c3n1)C2] | N-((1R,5S,8s)-3-(8-((3-methyl-4-((1- methyl-1H-benzo[d][1,2,3]triazol-5- yl)oxy)phenyl)amino)pyrimido[5,4- d]pyrimidin-2-yl)-3- azabicyclo[3.2.1]octan-8- yl)acrylamide | 563.3 (M + H) |
| 207 | 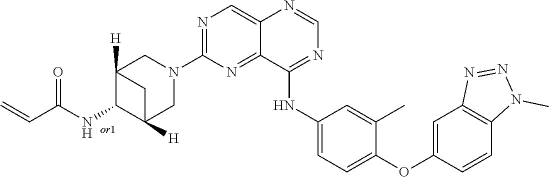 [SMILES: C=CC(=O)N[C@H]1[C@@H]2C[C@H]1CN(c1ncc3ncnc(Nc4ccc(Oc5ccc6c(c5)nnn6C)c(C)c4)c3n1)C2] | N-((1R,5S,6r)-3-(8-((3-methyl-4-((1- methyl-1H-benzo[d][1,2,3]triazol-5- yl)oxy)phenyl)amino)pyrimido[5,4- d]pyrimidin-2-yl)-3- azabicyclo[3.1.1]heptan-6- yl)acrylamide | 549.2 (M + H) |
| 208 | 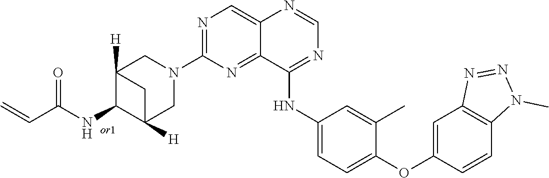 [SMILES: C=CC(=O)N[C@@H]1[C@@H]2C[C@H]1CN(c1ncc3ncnc(Nc4ccc(Oc5ccc6c(c5)nnn6C)c(C)c4)c3n1)C2] | N-((1R,5S,6s)-3-(8-((3-methyl-4-((1- methyl-1H-benzo[d][1,2,3]triazol-5- yl)oxy)phenyl)amino)pyrimido[5,4- d]pyrimidin-2-yl)-3- azabicyclo[3.1.1]heptan-6- yl)acrylamide | 549.3 (M + H) |

### Example 56

### 2-fluoro-1-(5-(8-((3-methyl-4-((1-methyl-1H-benzo[d][1,2,3]triazol-5-yl)oxy)phenyl)amino)pyrimido[5,4-d]pyrimidin-2-yl)-2,5-diazabicyclo[2.2.2]octan-2-yl)prop-2-en-1-one (compound 50)

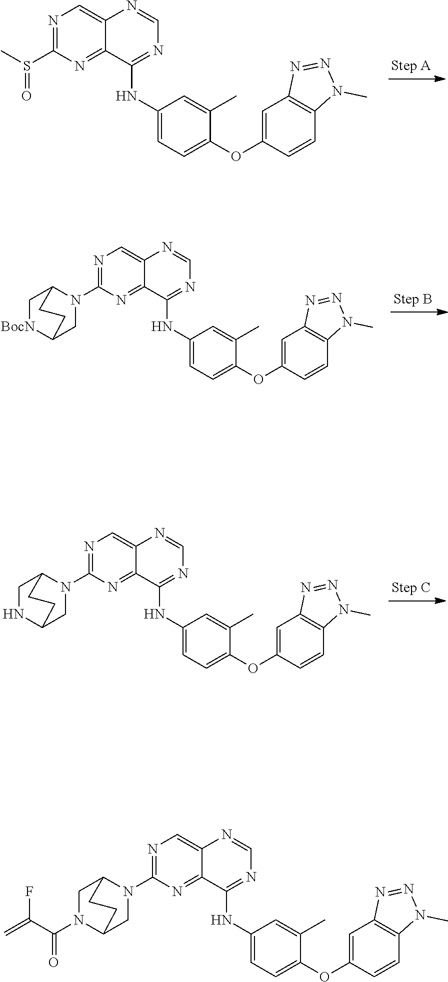

Step A. tert-butyl 5-(8-((3-methyl-4-((1-methyl-1H-benzo[d][11,2,3]triazol-5-yl)oxy)phenyl)amino)pyrimido[5,4-d]pyrimidin-2-yl)-2,5-diazabicyclo[2.2.2]octane-2-carboxylate. Prepared according to Example 55 Step A, substituting tert-butyl 2,5-diazabicyclo[2.2.2]octane-2-carboxylate for tert-butyl €-2-methylpiperazine-1-carboxylate tert-butyl 5-(8-((3-methyl-4-((1-methyl-1H-benzo[d][1,2,3]triazol-5-yl)oxy)phenyl)amino)pyrimido[5,4-d]pyrimidin-2-yl)-2,5-diazabicyclo[2.2.2]octane-2-carboxylate (44.8 mg, 80% yield) as a yellow solid. LCMS (MM-ES+APCI, Pos): m/z 595.3 (M+H).

Step B. 6-(2,5-diazabicyclo[2.2.2]octan-2-yl)-N-(3-methyl-4-((1-methyl-1H-benzo[d][1,2,3]triazol-5-yl)oxy)phenyl)pyrimido[5,4-d]pyrimidin-4-amine. Prepared according to Example 55 Step B, substituting tert-butyl 5-(8-((3-methyl-4-((1-methyl-1H-benzo[d][1,2,3]triazol-5-yl)oxy)phenyl)amino)pyrimido[5,4-d]pyrimidin-2-yl)-2,5-diazabicyclo[2.2.2]octane-2-carboxylate for tert-but€(R)-2-methyl-4-[4-[3-methyl-4-(1-methylbenzotriazol-5-yl)oxy-anilino]pyrimido[5,4-d]pyrimidin-6-yl]piperazine-1-carboxylate to afford 6-(2,5-diazabicyclo[2.2.2]octan-2-yl)-N-(3-methyl-4-((1-methyl-1H-benzo[d][1,2,3]triazol-5-yl)oxy)phenyl)pyrimido[5,4-d]pyrimidin-4-amine (38.1 mg, quant. yield). LCMS (MM-ES+APCI, Pos): m/z 495.3 (M+H).

Step C. 2-fluoro-1-(5-(8-((3-methyl-4-((1-methyl-1H-benzo[d][1,2,3]triazol-5-yl)oxy)phenyl)amino)pyrimido[5,4-d]pyrimidin-2-yl)-2,5-diazabicyclo[2.2.2]octan-2-yl)prop-2-en-1-one. 6-(2,5-diazabicyclo[2.2.2]octan-2-yl)-N-(3-methyl-4-((1-methyl-1H-benzo[d][1,2,3]triazol-5-yl)oxy)phenyl)pyrimido[5,4-d]pyrimidin-4-amine (38.4 mg, 0.078 mmol), 2-fluoroacrylic acid (15.4 mg, 0.171 mmol), DIPEA (0.07 mL, 0.4 mmol), and HATU (65.0 mg, 0.171 mmol) were diluted in DMF (1 mL). The reaction was stirred at room temperature for 1 h, partitioned between 20% MeOH/DCM (20 mL) and brine, and extracted with 20% MeOH/DCM (5×10 mL). The combined organic layers were washed with brine (3×10 mL), dried over anhydrous MgSO₄, and concentrated in vacuo. The crude residue was purified by silica gel chromatography (0% to 20% MeOH/DCM) to afford 2-fluoro-1-(5-(8-((3-methyl-4-((1-methyl-1H-benzo[d][1,2,3]triazol-5-yl)oxy)phenyl)amino)pyrimido[5,4-d]pyrimidin-2-yl)-2,5-diazabicyclo[2.2.2]octan-2-yl)prop-2-en-1-one (21.3 mg, 46% yield). LCMS (MM-ES+APCI, Pos): m/z 567.3 (M+H).

Compounds 50-59 in Table 5 below were prepared according to Example 56. *Racemic mixture separated by SFC.

TABLE 5
A list of compounds 50-59

| | | | MS |
|---|---|---|---|
| Cmp. | | | API |
| ID | Structure | Chemical Name | (m/z) |
| 50 | 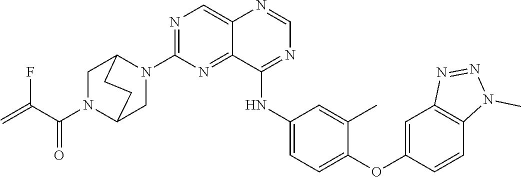 [SMILES: C=C(F)C(=O)N1CC2CCC1CN2c1ncc2ncnc(Nc3ccc(Oc4ccc5c(c4)nnn5C)c(C)c3)c2n1] | 2-fluoro-1-(5-(8- ((3-methyl-4- ((1-methyl-1H- benzo[d][1,2,3] triazol-5- yl)oxy)phenyl) amino)pyrimido [5,4-d]pyrimidin- 2-yl)-2,5- diazabicyclo [2.2.2]octan-2- yl)prop-2- en-1-one | 567.3 (M + H) |
| 51 | 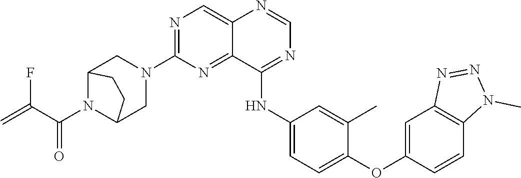 [SMILES: C=C(F)C(=O)N1C2CCC1CN(c1ncc3ncnc(Nc4ccc(Oc5ccc6c(c5)nnn6C)c(C)c4)c3n1)C2] | 2-fluoro-1-(3-(8- ((3-methyl-4- ((1-methyl-1H- benzo[d][1,2,3] triazol-5- yl)oxy)phenyl) amino)pyrimido [5,4-d]pyrimidin- 2-yl)-3,8- diazabicyclo [3.2.1]octan-8- yl)prop-2-en- 1-one 2,2,2- trifluoroacetate | 567.3 (M + H − TFA) |
| 52 | 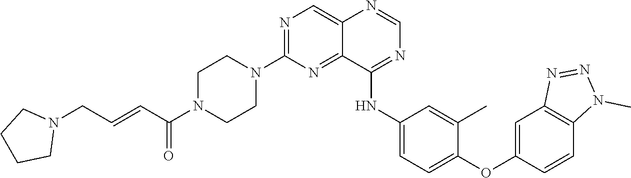 [SMILES: Cc1cc(Nc2ncnc3cnc(N4CCN(C(=O)/C=C/CN5CCCC5)CC4)nc23)ccc1Oc1ccc2c(c1)nnn2C] | (E)-1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1- y169yrrolidineolidin- 1-yl)but- 2-en-1-one | 606.3 (M + H) |
| 53 | 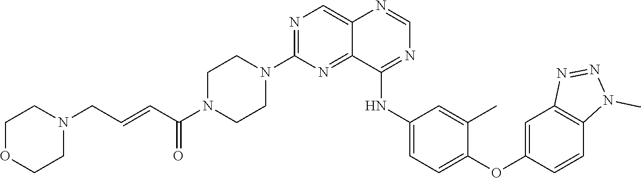 [SMILES: Cc1cc(Nc2ncnc3cnc(N4CCN(C(=O)/C=C/CN5CCOCC5)CC4)nc23)ccc1Oc1ccc2c(c1)nnn2C] | (E)-1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl)-4- morpholinobut- 2-en-1-one formate | 622.4 (M + H − FA) |
| 54 | 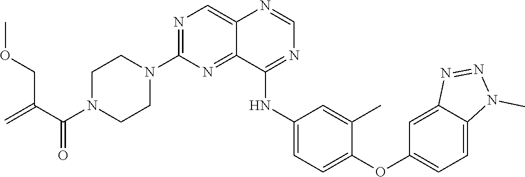 [SMILES: C=C(COC)C(=O)N1CCN(c2ncc3ncnc(Nc4ccc(Oc5ccc6c(c5)nnn6C)c(C)c4)c3n2)CC1] | 2-(methoxymethyl)- 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl) prop-2-en-1- one | 567.3 (M + H) |
| 55 | 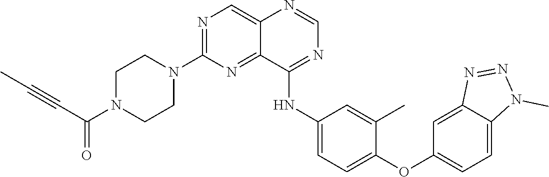 [SMILES: CC#CC(=O)N1CCN(c2ncc3ncnc(Nc4ccc(Oc5ccc6c(c5)nnn6C)c(C)c4)c3n2)CC1] | 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl) but-2-yn-1- one | 535.3 (M + H) |
| 56 | 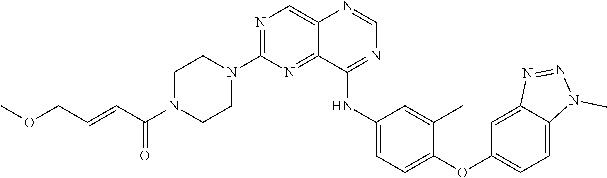 | (E)-4-methoxy- 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl) but-2-en-1- one | 567.3 (M + H) |
| 57 | 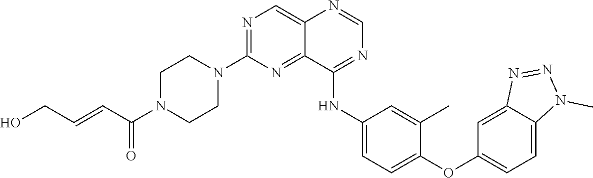 [SMILES: Cc1cc(Nc2ncnc3cnc(N4CCN(C(=O)/C=C/CO)CC4)nc23)ccc1Oc1ccc2c(c1)nnn2C] | (E)-4-hydroxy- 1-(4-(8-((3- methyl-4-((1- methyl-1H- benzo[d][1,2,3] triazol-5- yl)oxy)phenyl) amino)pyrimido [5,4-d] pyrimidin-2- yl)piperazin-1- yl)but-2-en-1- one | 553.3 (M + H) |
| 58 | 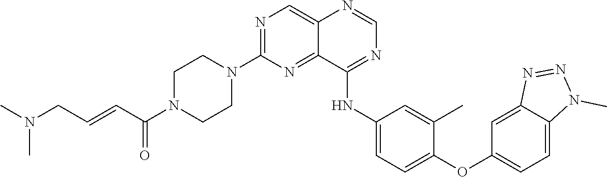 | (E)-4-(dimethyl- amino)-1-(4-(8- ((3-methyl-4- ((1-methyl-1H- benzo[d][1,2,3] triazol-5- yl)oxy)phenyl) amino)pyrimido [5,4-d]pyrimidin-2- yl)piperazin-1- yl)but-2-en-1- one | 580.4 (M + H) |
| 59 | 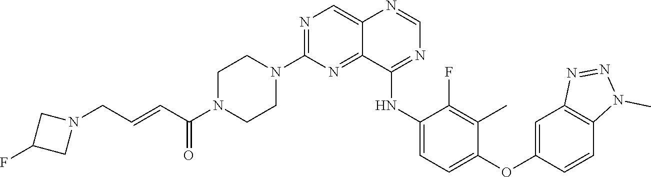 [SMILES: Cc1c(Oc2ccc3c(c2)nnn3C)ccc(Nc2ncnc3cnc(N4CCN(C(=O)/C=C/CN5CC(F)C5)CC4)nc23)c1F] | (E)-1-(4-(8- ((2-fluoro-3- methyl-4-((1- methyl-1H- benzo[d][1,2,3] triazol-5- yl)oxy)phenyl) amino)pyrimido [5,4-d]pyrimidin-2- yl)piperazin- 1-yl)-4-(3- fluoroazetidin-1- yl)but-2-en-1- one | 628.3 (M + H) |
| 209 | 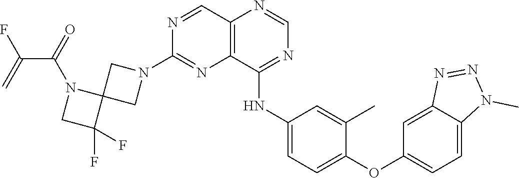 [SMILES: C=C(F)C(=O)N1CC(F)(F)C12CN(c1ncc3ncnc(Nc4ccc(Oc5ccc6c(c5)nnn6C)c(C)c4)c3n1)C2] | 1-(3,3-difluoro- 6-(8-((3- methyl-4-((1- methyl-1H- benzo[d][1,2,3] triazol-5- yl)oxy)phenyl) amino)pyrimido [5,4-d]pyrimidin- 2-yl)-1,6- diazaspiro[3.3] heptan-1-yl)-2- fluoroprop- 2-en-1-one | 589.2 (M + H) |
| 210 | 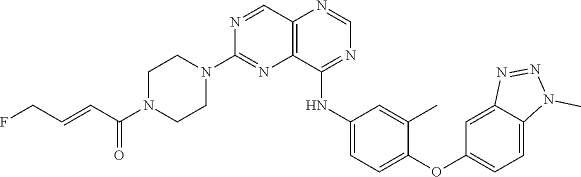 [SMILES: Cc1cc(Nc2ncnc3cnc(N4CCN(C(=O)/C=C/CF)CC4)nc23)ccc1Oc1ccc2c(c1)nnn2C] | (E)-4-fluoro- 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1- yl)but-2-en-1- one | 555.4 (M + H) |
| 211 | 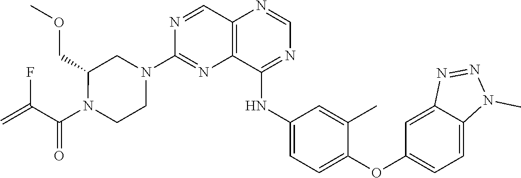 [SMILES: C=C(F)C(=O)N1CCN(c2ncc3ncnc(Nc4ccc(Oc5ccc6c(c5)nnn6C)c(C)c4)c3n2)C[C@H]1COC] | (S)-2-fluoro-1-(2- (methoxymethyl)- 4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl) prop-2-en-1- one | 585.4 (M + H) |
| 212 | 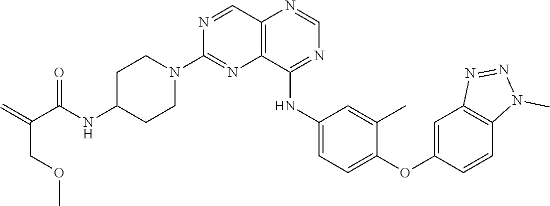 [SMILES: C=C(COC)C(=O)NC1CCN(c2ncc3ncnc(Nc4ccc(Oc5ccc6c(c5)nnn6C)c(C)c4)c3n2)CC1] | 2-(methoxymethyl)- N-(1-(8- ((3-methyl-4-((1- methyl-1H- benzo[d][1,2,3] triazol-5- yl)oxy)phenyl) amino)pyrimido [5,4-d]pyrimidin-2- yl)piperidin-4- yl)acrylamide | 581.3 (M + H) |
| 213 | 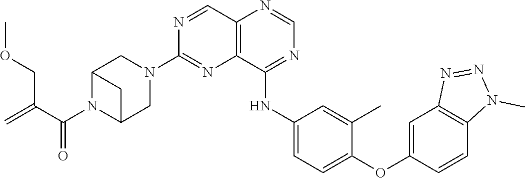 [SMILES: C=C(COC)C(=O)N1C2CC1CN(c1ncc3ncnc(Nc4ccc(Oc5ccc6c(c5)nnn6C)c(C)c4)c3n1)C2] | 2-(methoxymethyl)- 1-(3-(8-((3- methyl-4-((1- methyl-1H- benzo[d][1,2,3] triazol-5- yl)oxy)phenyl) amino)pyrimido [5,4-d]pyrimidin- 2-yl)-3,6- diazabicyclo[3.1.1] heptan-6- yl)prop-2- en-1-one | 579.4 (M + H) |
| 214 | 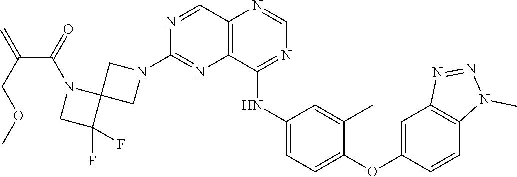 [SMILES: C=C(COC)C(=O)N1CC(F)(F)C12CN(c1ncc3ncnc(Nc4ccc(Oc5ccc6c(c5)nnn6C)c(C)c4)c3n1)C2] | 1-(3,3-difluoro- 6-(8-((3- methyl-4- ((1-methyl-1H- benzo[d][1,2,3] triazol-5- yl)oxy)phenyl) amino)pyrimido [5,4-d]pyrimidin- 2-yl)-1,6- diazaspiro[3.3] heptan-1-yl)-2- (methoxymethyl) prop-2-en-1- one | 615.2 (M + H) |
| 215 | 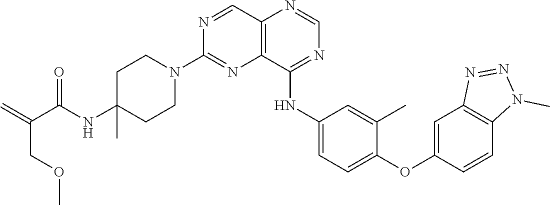 [SMILES: C=C(COC)C(=O)NC1(C)CCN(c2ncc3ncnc(Nc4ccc(Oc5ccc6c(c5)nnn6C)c(C)c4)c3n2)CC1] | 2-(methoxy- methyl)-N-(4- methyl-1-(8-((3- methyl-4-((1- methyl-1H- benzo[d][1,2,3] triazol-5- yl)oxy)phenyl) amino)pyrimido [5,4-d]pyrimidin-2- yl)piperidin-4- yl)acrylamide | 595.3 (M + H) |
| 216 | 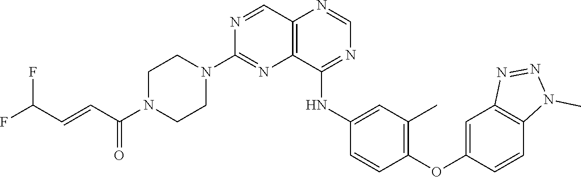 [SMILES: Cc1cc(Nc2ncnc3cnc(N4CCN(C(=O)/C=C/C(F)F)CC4)nc23)ccc1Oc1ccc2c(c1)nnn2C] | 4,4-difluoro- 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1- yl)but-2-en-1- one | 573.3 (M + H) |
| 217 | 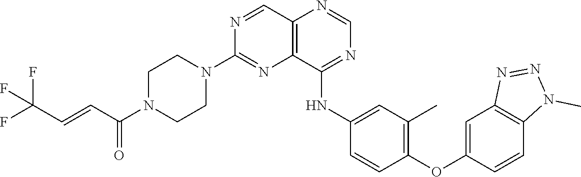 [SMILES: Cc1cc(Nc2ncnc3cnc(N4CCN(C(=O)/C=C/C(F)(F)F)CC4)nc23)ccc1Oc1ccc2c(c1)nnn2C] | 4,4,4-trifluoro- 1-(4-(8-((3- methyl-4-((1- methyl-1H- benzo[d][1,2,3] triazol-5- yl)oxy)phenyl) amino)pyrimido [5,4-d]pyrimidin-2- yl)piperazin-1-yl) but-2-en-1- one | 591.3 (M + H) |
| 218 | 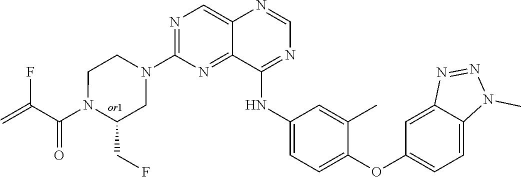 [SMILES: C=C(F)C(=O)N1CCN(c2ncc3ncnc(Nc4ccc(Oc5ccc6c(c5)nnn6C)c(C)c4)c3n2)C[C@@H]1CF] | (R)-2-fluoro-1-(2- (fluoromethyl)- 4-(8-((3- methyl-4-((1- methyl-1H- benzo[d][1,2,3] triazol-5- yl)oxy)phenyl) amino)pyrimido [5,4-d]pyrimidin-2- yl)piperazin-1- yl)prop-2-en-1- one* | 573.3 (M + H) |
| 219 | 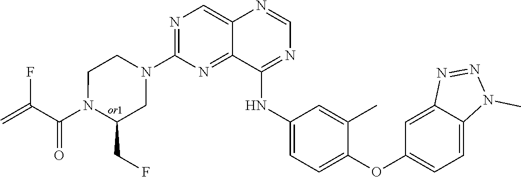 [SMILES: C=C(F)C(=O)N1CCN(c2ncc3ncnc(Nc4ccc(Oc5ccc6c(c5)nnn6C)c(C)c4)c3n2)C[C@H]1CF] | (S)-2-fluoro-1-(2- (fluoromethyl)- 4-(8-((3- methyl-4-((1- methyl-1H- benzo[d][1,2,3] triazol-5- yl)oxy)phenyl) amino)pyrimido [5,4-d] pyrimidin-2- yl)piperazin-1- yl)prop-2-en-1- one* | 573.3 (M + H) |

### Example 57

### 1-(3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptan-6-yl)prop-2-en-1-one (compound 60)

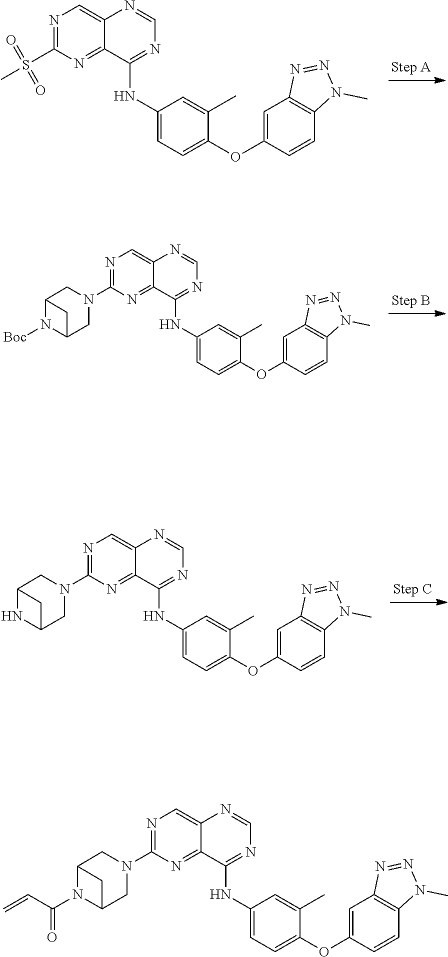

Step A. tert-butyl 3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptane-6-carboxylate. To a solution of N-(3-methyl-4-((1-methyl-1H-benzo[d][1,2,3]triazol-5-yl)oxy)phenyl)-6-(methylsulfonyl)pyrimido[5,4-d]pyrimidin-4-amine (200 mg, 0.43 mmol) and 6-(tert-butyloxycarbonyl)-3,6-diazabicyclo[3.1.1]heptane (171 mg, 0.86 mmol) in DMF (2.1 mL) was added K₂CO₃ (120 mg, 0.86 mmol). The mixture was stirred at 90° C. for 4 h, diluted with MeOH, and filtered. The filtrate was concentrated in vacuo and purified by preparative HPLC (5% to 95% MeCN/0.1% aqueous TFA). The purified product was neutralized with sat. aq. NaHCO₃ and extracted with 4:1 DCM:IPA to afford tert-butyl 3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptane-6-carboxylate (34 mg, 14% yield) as a yellow solid. LCMS (MM-ES+APCI, Pos): m/z 581.4 (M+H).

Step B: 6-(3,6-diazabicyclo[3.1.1]heptan-3-yl)-N-(3-methyl-4-((1-methyl-1H-benzo[d][1,2,3]triazol-5-yl)oxy)phenyl)pyrimido[5,4-d]pyrimidin-4-amine. To a solution of tert-butyl 3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptane-6-carboxylate (34 mg, 59 μmol) in DCM (1 mL) was added TFA (1 mL, 13 mmol). The mixture was stirred at 25° C. for 2 h and concentrated under reduced pressure. The residue was dissolved in 4:1 DCM:IPA and washed with sat. aq. NaHCO₃ (50 ml×2) and brine (50 ml×1). The organic layer was dried over anhydrous Na₂SO₄, filtered, and concentrated under reduced pressure. The crude material was purified by preparative HPLC (5% to 95% MeCN/0.1% aqueous TFA). The product was neutralized using sat. aq. NaHCO₃ and extracted with 4:1 DCM:IPA to afford 6-(3,6-diazabicyclo[3.1.1]heptan-3-yl)-N-(3-methyl-4-((1-methyl-1H-benzo[d][1,2,3]triazol-5-yl)oxy)phenyl)pyrimido[5,4-d]pyrimidin-4-amine (15 mg, 53%) as a yellow solid. LCMS (MM-ES+APCI, Pos): m/z 481.2 (M+H).

Step C. 1-(3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptan-6-yl)prop-2-en-1-one. To a solution of 6-(3,6-diazabicyclo[3.1.1]heptan-3-yl)-N-(3-methyl-4-((1-methyl-1H-benzo[d]1[1,2,3]triazol-5-yl)oxy)phenyl)pyrimido[5,4-d]pyrimidin-4-amine (15 mg, 31 μmol) in DCM (0.2 mL) was added TEA (8.7 μL, 62 μmol). The mixture was cooled to −78° C. and acryloyl chloride (5.6 μL, 69 μmol) was added dropwise. The reaction was warmed to 25° C., stirred for 0.5 h, and quenched with sat. aq. NaHCO₃ (0.2 mL). The reaction was diluted with additional sat. aq. NaHCO₃ and extracted with 4:1 DCM: IPA (50 ml×2). The combined organics were dried over anhydrous Na₂SO₄, filtered, and concentrated under reduced pressure. The residue was purified by preparative HPLC (5% to 95% MeCN/0.1% aqueous TFA). The product was neutralized using sat. aq. NaHCO₃ and extracted with 4:1 DCM:IPA to afford 1-(3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.1]heptan-6-yl)prop-2-en-1-one (6.9 mg, 41%) as a yellow solid. LCMS (MM-ES+APCI, Pos): m/z 535.3 (M+H).

Compounds 60-66 in Table 6 below were synthesized according to Example 57. Step A can be accomplished using K₂CO₃ or DIPEA as base, and DMF, DMSO, or IPA as solvent.

TABLE 6
A list of compounds 60-66

| | | | MS |
|---|---|---|---|
| Cmp. | | | API |
| ID | Structure | Chemical Name | (m/z) |
| 60 | 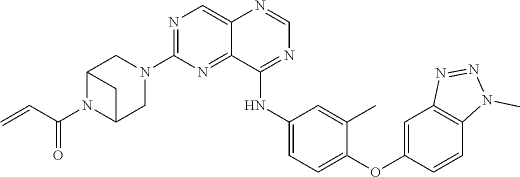 [SMILES: C=CC(=O)N1C2CC1CN(c1ncc3ncnc(Nc4ccc(Oc5ccc6c(c5)nnn6C)c(C)c4)c3n1)C2] | 1-(3-(8-((3-methyl-4-((1-methyl-1H- benzo[d][1,2,3]triazol-5- yl)oxy)phenyl)amino)pyrimido[5,4- d]pyrimidin-2-yl)-3,6- diazabicyclo[3.1.1]heptan-6-yl)prop- 2-en-1-one | 535.3 (M + H) |
| 61 | 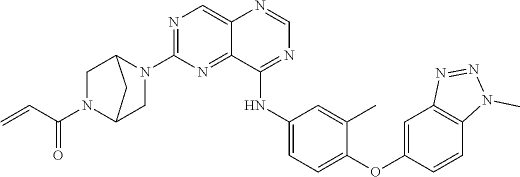 [SMILES: C=CC(=O)N1CC2CC1CN2c1ncc2ncnc(Nc3ccc(Oc4ccc5c(c4)nnn5C)c(C)c3)c2n1] | 1-(5-(8-((3-methyl-4-((1-methyl-1H- benzo[d][1,2,3]triazol-5- yl)oxy)phenyl)amino)pyrimido[5,4- d]pyrimidin-2-yl)-2,5- diazabicyclo[2.2.1 ]heptan-2-yl)prop- 2-en-1-one | 535.3 (M + H) |
| 62 | 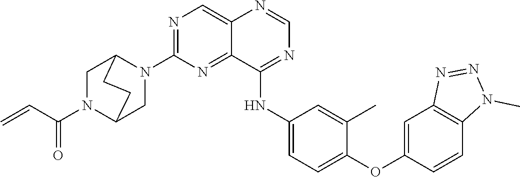 [SMILES: C=CC(=O)N1CC2CCC1CN2c1ncc2ncnc(Nc3ccc(Oc4ccc5c(c4)nnn5C)c(C)c3)c2n1] | 1-(5-(8-((3-methyl-4-((1-methyl-1H- benzo[d][1,2,3]triazol-5- yl)oxy)phenyl)amino)pyrimido[5,4- d]pyrimidin-2-yl)-2,5- diazabicyclo[2.2.2]octan-2-yl)prop- 2-en-1-one | 549.3 (M + H) |
| 63 | 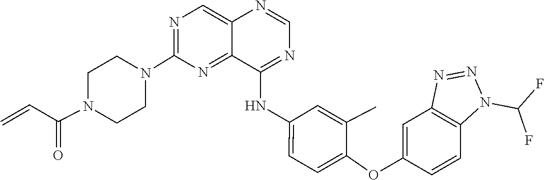 [SMILES: C=CC(=O)N1CCN(c2ncc3ncnc(Nc4ccc(Oc5ccc6c(c5)nnn6C(F)F)c(C)c4)c3n2)CC1] | 1-(4-(8-((4-((1-(difluoromethyl)-1H- benzo[d][1,2,3]triazol-5-yl)oxy)-3- methylphenyl)amino)pyrimido[5,4- d]pyrimidin-2-yl)piperazin-1- yl)prop-2-en-1-one | 559.4 (M + H) |
| 64 | 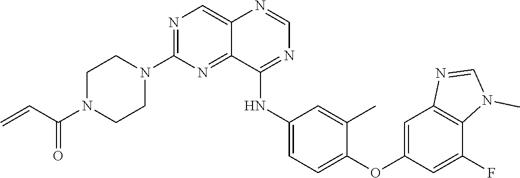 [SMILES: C=CC(=O)N1CCN(c2ncc3ncnc(Nc4ccc(Oc5cc(F)c6c(c5)ncn6C)c(C)c4)c3n2)CC1] | 1-(4-(8-((4-((7-fluoro-1-methyl-1H- benzo[d]imidazol-5-yl)oxy)-3- methylphenyl)amino)pyrimido[5,4- d]pyrimidin-2-yl)piperazin-1- yl)prop-2-en-1-one | 540.2 (M + H) |
| 65 | 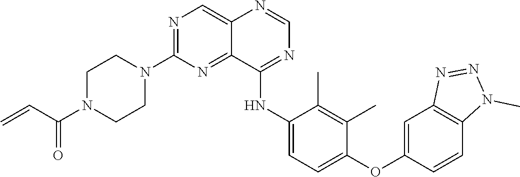 [SMILES: C=CC(=O)N1CCN(c2ncc3ncnc(Nc4ccc(Oc5ccc6c(c5)nnn6C)c(C)c4C)c3n2)CC1] | 1-[4-[4-[2,3-dimethyl-4-(1- methylbenzotriazol-5-yl)oxy- anilino]pyrimido[5,4-d]pyrimidin-6- yl]piperazin-1-yl] prop-2-en-1-one | 537.1 (M + H) |
| 66 | 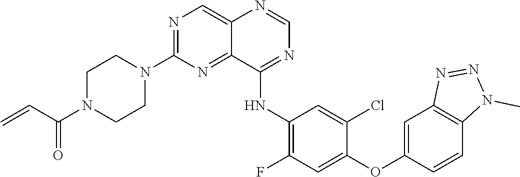 [SMILES: C=CC(=O)N1CCN(c2ncc3ncnc(Nc4cc(Cl)c(Oc5ccc6c(c5)nnn6C)cc4F)c3n2)CC1] | 1-(4-(8-((5-chloro-2-fluoro-4-((1- methyl-1H-benzo[d][1,2,3]triazol-5- yl)oxy)phenyl)amino)pyrimido[5,4- d]pyrimidin-2-yl)piperazin-1- yl)prop-2-en-1-one | 561.1 (M + H) |
| 220 | 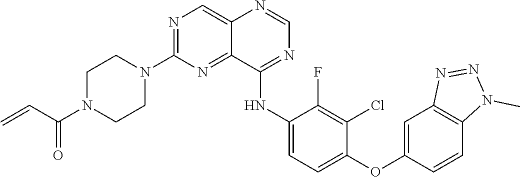 [SMILES: C=CC(=O)N1CCN(c2ncc3ncnc(Nc4ccc(Oc5ccc6c(c5)nnn6C)c(Cl)c4F)c3n2)CC1] | 1-(4-(8-((3-chloro-2-fluoro-4-((1- methyl-1H-benzo[d][1,2,3]triazol-5- yl)oxy)phenyl)amino)pyrimido[5,4- d]pyrimidin-2-yl)piperazin-1- yl)prop-2-en-1-one | 561.1 (M + H) |
| 221 | 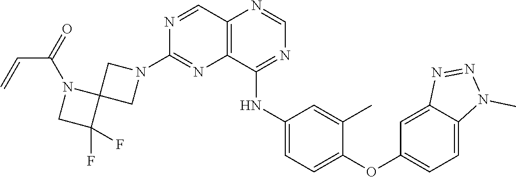 [SMILES: C=CC(=O)N1CC(F)(F)C12CN(c1ncc3ncnc(Nc4ccc(Oc5ccc6c(c5)nnn6C)c(C)c4)c3n1)C2] | 1-(3,3-difluoro-6-(8-((3-methyl-4- ((1-methyl-1H- benzo[d][1,2,3]triazol-5- yl)oxy)phenyl)amino)pyrimido[5,4- d]pyrimidin-2-yl)-1,6- diazaspiro[3.3]heptan-1-yl)prop-2- en-1-one | 571.2 (M + H) |
| 222 | 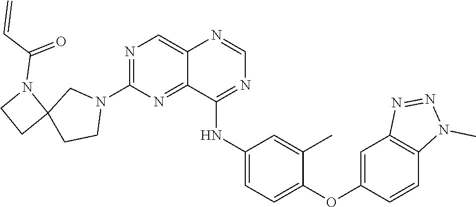 [SMILES: C=CC(=O)N1CCC12CCN(c1ncc3ncnc(Nc4ccc(Oc5ccc6c(c5)nnn6C)c(C)c4)c3n1)C2] | 1-(6-(8-((3-methyl-4-((1-methyl-1H- benzo[d][1,2,3]triazol-5- yl)oxy)phenyl)amino)pyrimido[5,4- d]pyrimidin-2-yl)-1,6- diazaspiro[3.4]octan-1-yl)prop-2-en- 1-one | 549.4 (M + H) |
| 223 | 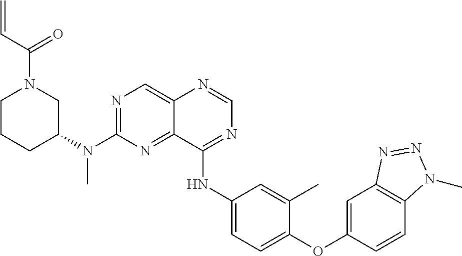 [SMILES: C=CC(=O)N1CCC[C@@H](N(C)c2ncc3ncnc(Nc4ccc(Oc5ccc6c(c5)nnn6C)c(C)c4)c3n2)C1] | (R)-1-(3-(methyl(8-((3-methyl-4-((1- methyl-1H-benzo[d][1,2,3]triazol-5- yl)oxy)phenyl)amino)pyrimido[5,4- d]pyrimidin-2-yl)amino)piperidin-1- yl)prop-2-en-1-one | 551.3 (M + H) |
| 224 | 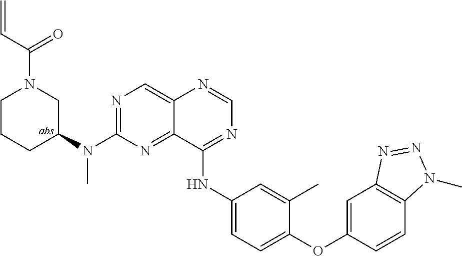 [SMILES: C=CC(=O)N1CCC[C@H](N(C)c2ncc3ncnc(Nc4ccc(Oc5ccc6c(c5)nnn6C)c(C)c4)c3n2)C1] | (S)-1-(3-(methyl(8-((3-methyl-4-((1- methyl-1H-benzo[d][1,2,3]triazol-5- yl)oxy)phenyl)amino)pyrimido[5,4- d]pyrimidin-2-yl)amino)piperidin-1- yl)prop-2-en-1-one | 551.4 (M + H) |

### Example 58

### N-(2-(8-((3-methyl-4-((1-methyl-1H-benzo[d][1,2,3]triazol-5-yl)oxy)phenyl)amino)pyrimido[5,4-d]pyrimidin-2-yl)-2-azaspiro[3.3]heptan-6-yl)acrylamide (compound 225)

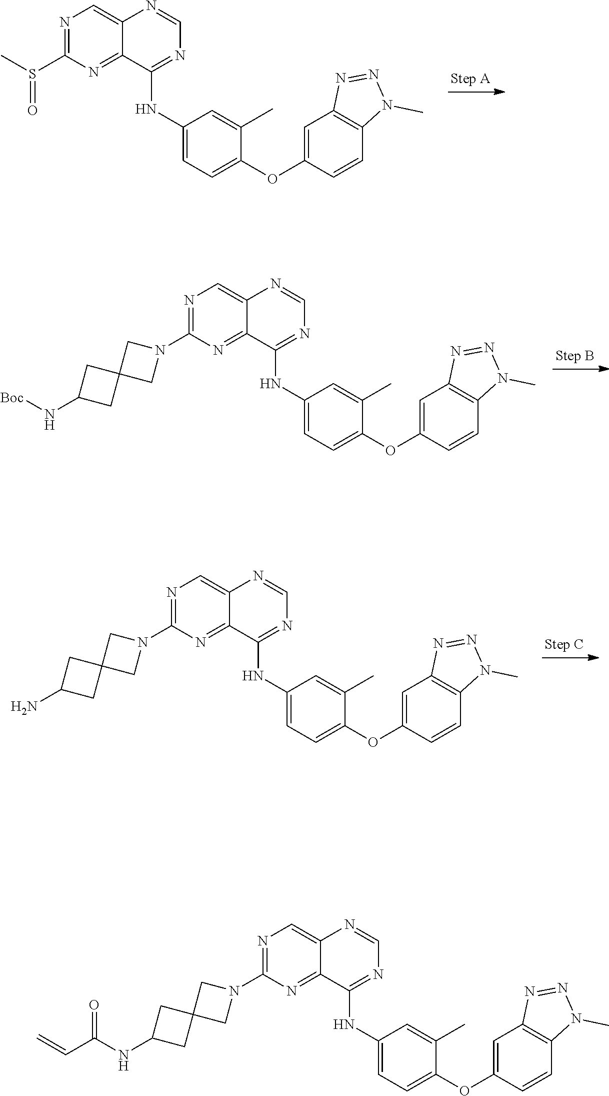

Step A. tert-butyl (2-(8-((3-methyl-4-((1-methyl-1H-benzo[d][1,2,3]triazol-5-yl)oxy)phenyl)amino)pyrimido[5,4-d]pyrimidin-2-yl)-2-azaspiro[3.3]heptan-6-yl)carbamate. To a solution of tert-butyl N-(2-azaspiro[3.3]heptan-6-yl)carbamate (200 mg, 0.94 mmol) and N-[3-methyl-4-(1-methylbenzotriazol-5-yl)oxy-phenyl]-6-methylsulfinyl-pyrimido[5,4-d]pyrimidin-4-amine (421 mg, 0.94 mmol) in IPA (10 mL) was added DIPEA (0.82 mL), 4.71 mmol). The reaction was heated to 80° C. for 15 h. The mixture was concentrated under reduced pressure and the residue was purified by silica gel column chromatography (50% to 100% EtOAc/PET) to afford tert-butyl N-[2-[4-[3-methyl-4-(1-methylbenzotriazol-5-yl)oxy-anilino]pyrimido[5,4-d]pyrimidin-6-yl]-2-azaspiro[3.3]heptan-6-yl]carbamate (328 mg, 59% yield) as a yellow solid. LCMS (MM-ES+APCI, Pos): m/z 595.6 (M+H).

Step B. 6-(6-amino-2-azaspiro[3.3]heptan-2-yl)-N-(3-methyl-4-((1-methyl-1H-benzo[d][1,2,3]triazol-5-yl)oxy)phenyl)pyrimido[5,4-d]pyrimidin-4-amine. To a solution of tert-butyl N-[2-[4-[3-methyl-4-(1-methylbenzotriazol-5-yl)oxy-anilino]pyrimido[5,4-d]pyrimidin-6-yl]-2-azaspiro[3.3]heptan-6-yl]carbamate (318 mg, 0.53 mmol) in DCM (10 mL) was added TFA (4 mL). The mixture was stirred at 20° C. for 20 min and concentrated in vacuo. The residue was dissolved in water (6 mL), neutralized (pH=7) with sat. aq. NaHCO₃ and extracted with EtOAc (20 mL×3). The combined organic layers were washed with brine (50 mL), dried over anhydrous sodium sulfate, filtered, and concentrated under reduced pressure to afford 6-(6-amino-2-azaspiro[3.3]heptan-2-yl)-N-[3-methyl-4-(1-methylbenzotriazol-5-yl)oxy-phenyl]pyrimido[5,4-d]pyrimidin-4-amine 4 (130 mg, 262.87 mol, 49.16% yield) was obtained as a yellow solid and was used into the next step without further purification.

### Example 59

### 1-(6-(8-((2-fluoro-3-methyl-4-((1-methyl-1H-benzo[d][1,2,3]triazol-5-yl)oxy)phenyl)amino)pyrimido[5,4-d]pyrimidin-2-yl)-1,6-diazaspiro[3.3]heptan-1-yl)prop-2-en-1-one (compound 67)

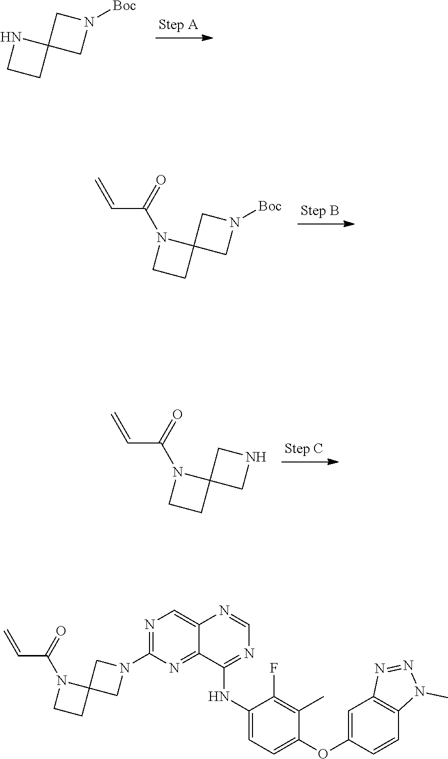 [SMILES: CC(C)(C)OC(=O)N1CC2(CCN2)C1 ; C=CC(=O)N1CCC12CN(C(=O)OC(C)(C)C)C2 ; C=CC(=O)N1CCC12CNC2 ; C=CC(=O)N1CCC12CN(c1ncc3ncnc(Nc4ccc(Oc5ccc6c(c5)nnn6C)c(C)c4F)c3n1)C2]

Step A. tert-butyl 1-acryloyl-1,6-diazaspiro[3.3]heptane-6-carboxylate. To a solution of tert-butyl 1,6-diazaspiro[3.3]heptane-6-carboxylate oxalate (80 mg, 0.28 mmol) in DCM (2 mL) was added TEA (0.12 mL, 0.83 mmol) at 25° C. The reaction was cooled to −40° C., prop-2-enoyl chloride (23 μL, 0.28 mmol) was added, and the mixture was stirred for 1 h. The reaction was quenched with sat. aq. NaHCO₃ (0.1 mL), extracted with DCM (10 mL×3), and the combined organic layers were dried over anhydrous Na₂SO₄, filtered, and concentrated under reduced pressure to afford tert-butyl 1-prop-2-enoyl-1,6-diazaspiro[3.3]heptane-6-carboxylate (60 mg, 86% yield) as a light yellow oil. LCMS (MM-ES+APCI, Pos): m/z 253.1 (M+H).

Step B. 1-(1,6-diazaspiro[3.3]heptan-1-yl)prop-2-en-1-one 2,2,2-trifluoroacetate. To a solution of tert-butyl 1-prop-2-enoyl-1, 6-diazaspiro[3.3]heptane-6-carboxylate (60 mg, 0.24 mmol) in DCM (1 mL) was added TFA (18 μL, 0.24 mmol). The mixture was stirred at 25° C. for 1 h and concentrated in vacuo to yield 1-(1,6-diazaspiro[3.3]heptan-1-yl)prop-2-en-1-one 2,2,2-trifluoroacetate (62 mg, quant. yield) as a light yellow oil. LCMS (MM-ES+APCI, Pos): m/z 153.1 (M+H-TFA).

Step C. 1-(6-(8-((2-fluoro-3-methyl-4-((1-methyl-1H-benzo[d][1,2,3]triazol-5-yl)oxy)phenyl)amino)pyrimido[5,4-d]pyrimidin-2-yl)-1,6-diazaspiro[3.3]heptan-1-yl)prop-2-en-1-one. To a solution of N-[2-fluoro-3-methyl-4-(1-methylbenzotriazol-5-yl)oxy-phenyl]-6-methylsulfinyl-pyrimido[5,4-d]pyrimidin-4-amine (50 mg, 0.11 mmol) and 1-(1,6-diazaspiro[3.3]heptan-1-yl)prop-2-en-1-one 2,2,2-trifluoroacetate (62 mg, 0.23 mmol) in IPA (3 mL) was added DIPEA (42 mg, 0.32 mmol). The mixture was stirred at 80° C. for 2 h and concentrated in vacuo. The crude material was purified by preparative HPLC (25% to 55% MeCN/0.08% aqueous NH₄HCO₃) to afford 1-[6-[4-[2-fluoro-3-methyl-4-(1-methylbenzotriazol-5-yl)oxy-anilino]pyrimido[5,4-d]pyrimidin-6-yl]-1,6-diazaspiro[3.3]heptan-1-yl]prop-2-en-1-one (13 mg, 22% yield) as a yellow solid. LCMS (MM-ES+APCI, Pos): m/z 553.2 (M+H).

Compounds 67, 68, 226-230 in Table 17 below were synthesized according to Example 59 steps A to C. *Racemic mixture separated by SFC.

TABLE 17
A list of compounds 67, 68, 226-230

| | | | MS |
|---|---|---|---|
| Cmp. | | | API |
| ID | Structure | Chemical Name | (m/z) |
| 67 | 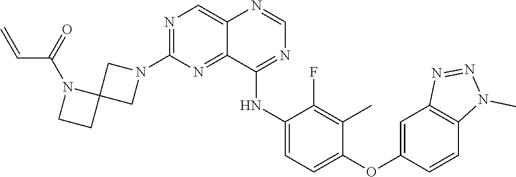 [SMILES: C=CC(=O)N1CCC12CN(c1ncc3ncnc(Nc4ccc(Oc5ccc6c(c5)nnn6C)c(C)c4F)c3n1)C2] | 1-(6-(8-((2-fluoro-3-methyl-4-((1- methyl-1H-benzo[d][1,2,3]triazol-5- yl)oxy)phenyl)amino)pyrimido[5,4- d]pyrimidin-2-yl)-1,6- diazaspiro[3.3]heptan-1-yl)prop-2-en- 1-one | 553.2 (M + H) |
| 68 | 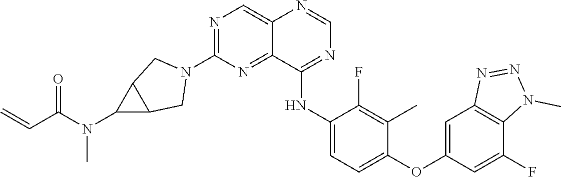 [SMILES: C=CC(=O)N(C)C1C2CN(c3ncc4ncnc(Nc5ccc(Oc6cc(F)c7c(c6)nnn7C)c(C)c5F)c4n3)CC21] | N-(3-(8-((2-fluoro-4-((7-fluoro-1- methyl-1H-benzo[d][1,2,3]triazol-5- yl)oxy)-3- methylphenyl)amino)pyrimido[5,4- d]pyrimidin-2-yl)-3- azabicyclo[3.1.0]hexan-6-yl)-N- methylacrylamide | 585.4 (M + H) |
| 226 | 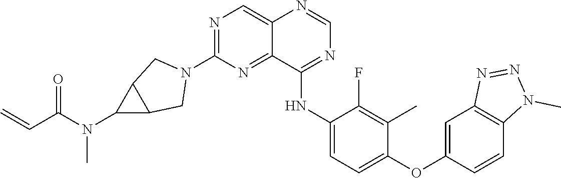 [SMILES: C=CC(=O)N(C)C1C2CN(c3ncc4ncnc(Nc5ccc(Oc6ccc7c(c6)nnn7C)c(C)c5F)c4n3)CC21] | N-(3-(8-((2-fluoro-3-methyl-4-((1- methyl-1H-benzo[d][1,2,3]triazol-5- yl)oxy)phenyl)amino)pyrimido[5,4- d]pyrimidin-2-yl)-3- azabicyclo[3.1.0]hexan-6-yl)-N- methylacrylamide | 567.3 (M + H) |
| 227 | 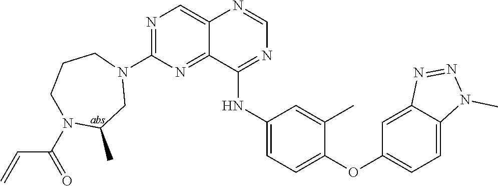 [SMILES: C=CC(=O)N1CCCN(c2ncc3ncnc(Nc4ccc(Oc5ccc6c(c5)nnn6C)c(C)c4)c3n2)C[C@H]1C] | (R)-1-(2-methyl-4-(8-((3-methyl-4- ((1-methyl-1H-benzo[d][1,2,3]triazol- 5-yl)oxy)phenyl)amino)pyrimido[5,4- d]pyrimidin-2-yl)-1,4-diazepan-1- yl)prop-2-en-1-one | 551.2 (M + H) |
| 228 | 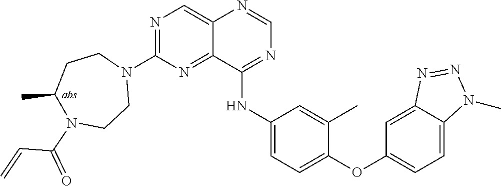 [SMILES: C=CC(=O)N1CCN(c2ncc3ncnc(Nc4ccc(Oc5ccc6c(c5)nnn6C)c(C)c4)c3n2)CC[C@@H]1C] | (S)-1-(7-methyl-4-(8-((3-methyl-4-((1- methyl-1H-benzo[d][1,2,3]triazol-5- yl)oxy)phenyl)amino)pyrimido[5,4- d]pyrimidin-2-yl)-1,4-diazepan-1- yl)prop-2-en-1-one | 551.0 (M + H) |
| 229 | 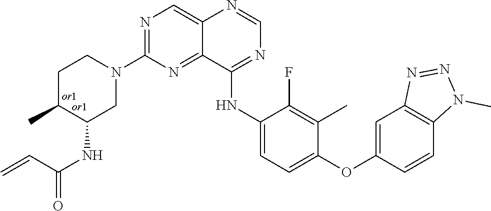 [SMILES: C=CC(=O)N[C@H]1CN(c2ncc3ncnc(Nc4ccc(Oc5ccc6c(c5)nnn6C)c(C)c4F)c3n2)CC[C@@H]1C] | N-((3R,4S)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-4-methylpiperidin- 3-yl)acrylamide* | 569.3 (M + H) |
| 230 | 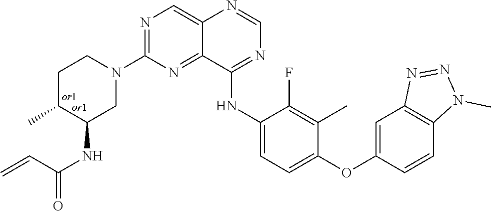 [SMILES: C=CC(=O)N[C@@H]1CN(c2ncc3ncnc(Nc4ccc(Oc5ccc6c(c5)nnn6C)c(C)c4F)c3n2)CC[C@H]1C] | N-((3S,4R)-1-(8-((2-fluoro-3-methyl- 4-((1-methyl-1H- benzo[d][1,2,3]triazol-5- yl)oxy)phenyl)amino)pyrimido[5,4- d]pyrimidin-2-yl)-4-methylpiperidin- 3-yl)acrylamide* | 569.3 (M + H) |

### Example 60

### (R)-2-fluoro-1l-(4-(8-((3-methyl-4-((1-methyl-1H-benzo[d][1,2,3]triazol-5-yl)oxy)phenyl)amino)pyrimido[5,4-d]pyrimidin-2-yl)-2-(trifluoromethyl)piperazin-1-yl)prop-2-en-1-one (compound 231)

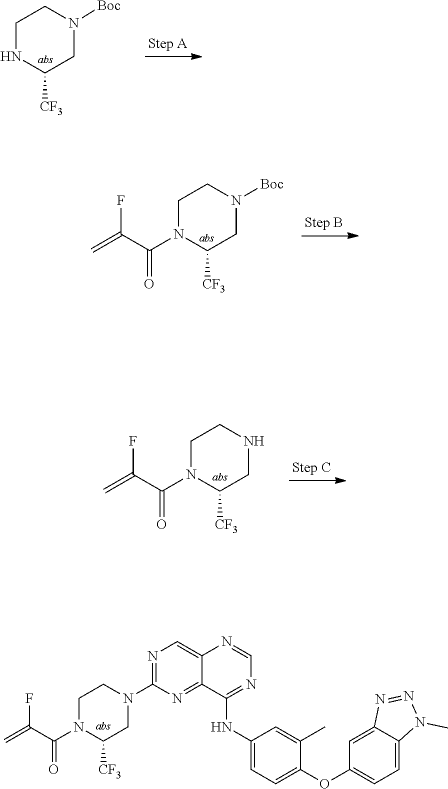 [SMILES: CC(C)(C)OC(=O)N1CCN[C@@H](C(F)(F)F)C1 ; C=C(F)C(=O)N1CCN(C(=O)OC(C)(C)C)C[C@@H]1C(F)(F)F ; C=C(F)C(=O)N1CCNC[C@@H]1C(F)(F)F ; C=C(F)C(=O)N1CCN(c2ncc3ncnc(Nc4ccc(Oc5ccc6c(c5)nnn6C)c(C)c4)c3n2)C[C@@H]1C(F)(F)F]

Step A. tert-butyl (R)-4-(2-fluoroacryloyl)-3-(trifluoromethyl)piperazine-1-carboxylate. To a solution of tert-butyl (3R)-3-(trifluoromethyl)piperazine-1-carboxylate (100 mg, 0.39 mmol) in DCM (2 mL) were added DIPEA (0.21 mL, 1.18 mmol) and 2-fluoroprop-2-enoyl chloride (43 mg, 0.39 mmol). The mixture was stirred at 25° C. for 1 h. The reaction was quenched with water (20 mL) and extracted with DCM (20 mL×3). The combined organic phases were washed with brine (20 mL), dried over anhydrous sodium sulfate, filtered, and concentrated under reduced. The crude residue was purified by preparative TLC (25% EtOAc/PET) to afford tert-butyl (3R)-4-(2-fluoroprop-2-enoyl)-3-(trifluoromethyl)piperazine-1-carboxylate 3 (122 mg, 95% yield) as a white solid. LCMS (MM-ES+APCI, Pos): m/z 271.1 (M+H-^{t}Bu).

Step B. (R)-2-fluoro-1-(2-(trifluoromethyl)piperazin-1-yl)prop-2-en-1-one. A solution of tert-butyl (3R)-4-(2-fluoroprop-2-enoyl)-3-(trifluoromethyl)piperazine-1-carboxylate (120 mg, 0.37 mmol) in DCM (2 mL) and TFA (1 mL) was stirred at 25° C. for 30 min. The mixture was concentrated under reduced pressure to afford 2-fluoro-1-[(2R)-2-(trifluoromethyl) piperazin-1-yl]prop-2-en-1-one trifluoroacetate (100 mg, 79% yield) as a yellow oil. LCMS (MM-ES+APCI, Pos): m/z 227.0 (M+H).

Step C. (R)-2-fluoro-1-(4-(8-((3-methyl-4-((1-methyl-1H-benzo[d][1,2,3]triazol-5-yl)oxy)phenyl)amino)pyrimido[5,4-d]pyrimidin-2-yl)-2-(trifluoromethyl)piperazin-1-yl)prop-2-en-1-one. To a solution of 6-chloro-N-[3-methyl-4-(1-methylbenzotriazol-5-yl)oxy-phenyl]pyrimido[5,4-d]pyrimidin-4-amine (163 mg, 0.36 mmol) and 2-fluoro-1-[(2R)-2-(trifluoromethyl)piperazin-1-yl]prop-2-en-1-one trifluoroacetate (81 mg, 0.24 mmol) in IPA (5 mL) were added DIPEA (0.19 mL, 1.07 mmol) and KF (42 mg, 0.72 mmol). The reaction was stirred at 80° C. for 12 h. The mixture was concentrated under reduced pressure, diluted with water (20 mL), and extracted with EtOAc (15 mL×3). The combined organic layers were washed with brine (20 mL), dried over anhydrous sodium sulfate, filtered, and concentrated under reduced pressure. The residue was purified by preparative HPLC (38% to 68% MeCN/0.08% aqueous NH₄HCO₃) to afford 2-fluoro-1-[(2R)-4-[4-[3-methyl-4-(1-methylbenzotriazol-5-yl)oxyanilino]pyrimido[5,4-d]pyrimidin-6-yl]-2 (trifluoromethyl)piperazin-1-yl]prop-2-en-1-one (55.2 mg, 24% yield) as a yellow solid. LCMS (MM-ES+APCI, Pos): m/z 609.3 (M+H).

### Example 61

### 1-(3-((8-((2-fluoro-3-methyl-4-((1-methyl-1H-benzo[d][1,2,3]triazol-5-yl)oxy)phenyl)amino)pyrimido[5,4-d]pyrimidin-2-yl)(methyl)amino)azetidin-1-yl)prop-2-en-1-one (compound 69)

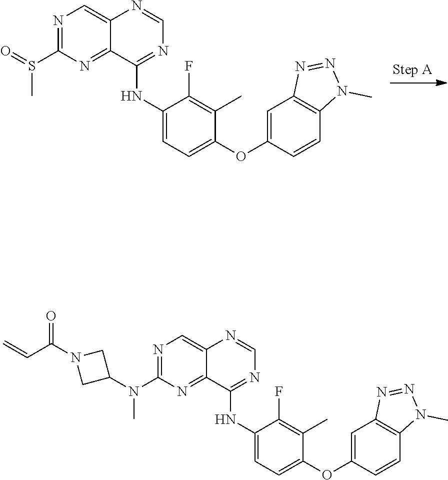

Step A. 1-(3-((8-((2-fluoro-3-methyl-4-((1-methyl-1H-benzo[d][1,2,3]triazol-5-yl)oxy)phenyl)amino)pyrimido[5,4-d]pyrimidin-2-yl)(methyl)amino)azetidin-1-yl)prop-2-en-1-one. Prepared according to Example 59 Step C, substituting 1-[3-(methylamino)azetidin-1-yl]prop-2-en-1-one 2,2,2-trifluoroacetate for 1-(1,6-diazaspiro[3.3]heptan-1-yl)prop-2-en-1-one 2,2,2-trifluoroacetate to provide 1-[3-[[4-[2-fluoro-3-methyl-4-(1-methylbenzotriazol-5-yl)oxy-anilino]pyrimido[5,4-d]pyrimidin-6-yl]-methyl-amino]azetidin-1-yl]prop-2-en-1-one (8 mg, 5% yield) as a yellow solid. LCMS (MM-ES+APCI, Pos): m/z 541.2 (M+H).

### Example 62

### N-methyl-N-(2-(8-((3-methyl-4-((1-methyl-H-benzo[d][1,2,3]triazol-5-yl)oxy)phenyl)amino)pyrimido[5,4-d]pyrimidin-2-yl)-2-azaspiro[3.3]heptan-6-yl)acrylamide (compound 232)

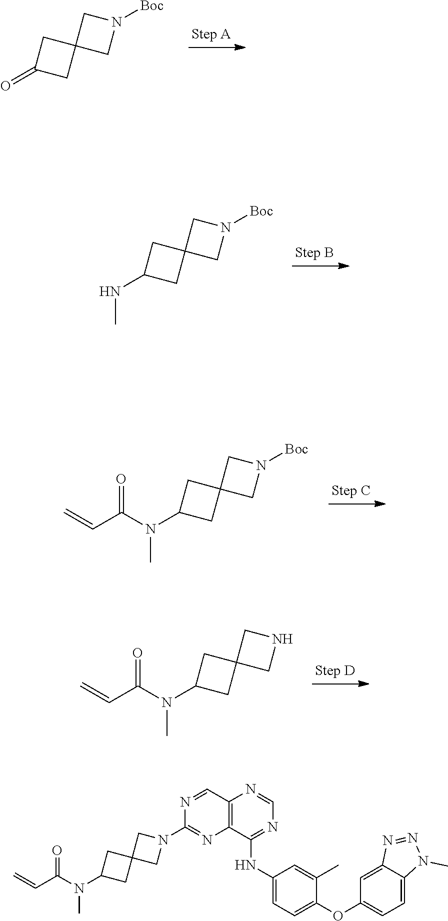 [SMILES: CC(C)(C)OC(=O)N1CC2(CC(=O)C2)C1 ; CNC1CC2(C1)CN(C(=O)OC(C)(C)C)C2 ; C=CC(=O)N(C)C1CC2(C1)CN(C(=O)OC(C)(C)C)C2 ; C=CC(=O)N(C)C1CC2(CNC2)C1 ; C=CC(=O)N(C)C1CC2(C1)CN(c1ncc3ncnc(Nc4ccc(Oc5ccc6c(c5)nnn6C)c(C)c4)c3n1)C2]

Step A. tert-butyl 6-(methylamino)-2-azaspiro[3.3]heptane-2-carboxylate. To a solution of methanamine hydrochloride (160 mg, 2.37 mmol) and TEA (0.49 mL, 3.55 mmol) in MeOH (10 mL) were added AcOH (7 μL, 0.12 mmol), tert-butyl 6-oxo-2-azaspiro[3.3]heptane-2-carboxylate (250 mg, 1.18 mmol) and NaBH₃CN (223 mg, 3.55 mmol). The mixture was stirred at 20° C. for 2 h. The reaction was quenched with water (20 mL) and extracted with EtOAc (30 mL×3). The combined organic layers were washed with brine (30 mL), dried over anhydrous sodium sulfate, filtered, and concentrated under reduced pressure to afford tert-butyl 6-(methylamino)-2-azaspiro[3.3]heptane-2-carboxylate 2 (215 mg, 80% yield) as a white solid. LCMS (MM-ES+APCI, Pos): m/z 227.4 (M+H).

Step B to Step D. N-methyl-N-(2-(8-((3-methyl-4-((1-methyl-1H-benzo[d][1,2,3]triazol-5-yl)oxy)phenyl)amino)pyrimido[5,4-d]pyrimidin-2-yl)-2-azaspiro[3.3]heptan-6-yl)acrylamide. Prepared according to Example 59 steps A to C, substituting tert-butyl 6-(methylamino)-2-azaspiro[3.3]heptane-2-carboxylate for tert-butyl 1,6-diazaspiro[3.3]heptane-6-carboxylate to afford N-methyl-N-(2-(8-((3-methyl-4-((1-methyl-1H-benzo[d][1,2,3]triazol-5-yl)oxy)phenyl)amino)pyrimido[5,4-d]pyrimidin-2-yl)-2-azaspiro[3.3]heptan-6-yl)acrylamide (15 mg, 12% yield) as a yellow solid. LCMS (MM-ES+APCI, Pos): m/z 563.4 (M+H).

### Example 63

### (E)-1-(4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)-4-(1H-pyrazol-1-yl)but-2-en-1-one (compound 233)

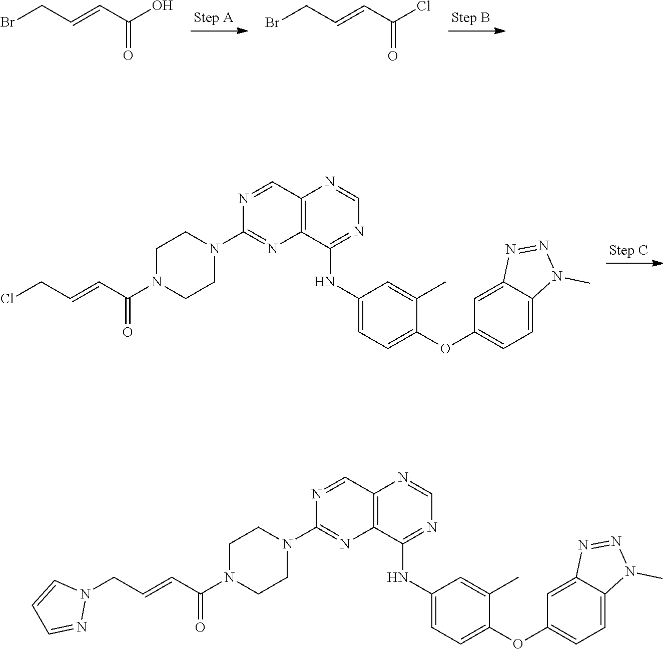 [SMILES: O=C(O)/C=C/CBr ; O=C(Cl)/C=C/CBr ; Cc1cc(Nc2ncnc3cnc(N4CCN(C(=O)/C=C/CCl)CC4)nc23)ccc1Oc1ccc2c(c1)nnn2C ; Cc1cc(Nc2ncnc3cnc(N4CCN(C(=O)/C=C/Cn5cccn5)CC4)nc23)ccc1Oc1ccc2c(c1)nnn2C]

Step A. (E)-4-bromobut-2-enoyl chloride. To a solution of (E)-4-bromobut-2-enoic acid 4 (200 mg, 1.21 mmol) in DCM (4 mL) was added oxalyl dichloride (0.12 mL, 1.33 mmol). The mixture was cooled to 0° C. and DMF (1 drop) was added. The mixture was allowed to warm to 20° C. and stirred for 2 h. The solution of (E)-4-bromobut-2-enoyl chloride 2 (222.36 mg, crude) in DCM (4 mL) was obtained as a brown liquid and used as is in step B.

Step B. (E)-4-chloro-1-(4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)but-2-en-1-one. To a solution of N-[3-methyl-4-(1-methylbenzotriazol-5-yl)oxy-phenyl]-6-piperazin-1-yl-pyrimido[5,4-d]pyrimidin-4-amine hydrochloride (100 mg, 0.20 mmol) in DCM (1 mL) was added DIPEA (86 μL, 0.50 mmol). The mixture was cooled to 0° C. and (E)-4-bromobut-2-enoyl chloride (73 mg, 0.40 mmol) was added dropwise. The reaction was warmed to ambient temperature, stirred for 2 h, and concentrated in vacuo. The residue was purified by preparatory silica gel TLC (5% MeOH/DCM) to afford (E)-4-chloro-1-[4-[4-[3-methyl-4-(1-methylbenzotriazol-5-yl)oxy-anilino]pyrimido[5,4-d]pyrimidin-6-yl]piperazin-1-yl]but-2-en-1-one (22 mg, 19% yield) as a yellow solid. LCMS (MM-ES+APCI, Pos): m/z 571.2 (M+H).

Step C. A solution of 1H-pyrazole (4.7 mg, 68 μmol) in THF (0.5 mL) was cooled to 0° C. and NaH (2.7 mg, 68 μmol, 60% w/w) was added. The mixture was warmed to ambient temperature and stirred for 30 min. (E)-4-chloro-1-[4-[4-[3-methyl-4-(1-methylbenzotriazol-5-yl)oxy-anilino]pyrimido[5,4-d]pyrimidi-n-6-yl]piperazin-1-yl]but-2-en-1-one (20.4 mg, 34 mol) was added and the mixture was stirred another 30 min. The reaction was quenched with sat. aq. NH₄Cl (0.2 mL), diluted with water, and extracted with EtOAc (2 mL×3). The combined organic layers were washed with brine (2 mL), dried over anhydrous sodium sulfate, filtered, and concentrated under reduced pressure. The crude residue was purified by preparative HPLC (30% to 60% MeCN/0.08% aqueous NH₄HCO₃) twice and lyophilized to afford (E)-1-[4-[4-[3-methyl-4-(1-methylbenzotriazol-5-yl)oxy-anilino]pyrimido[5,4-d]pyrimidin-6-yl]piperazin-1-yl]-4-pyrazol-1-yl-but-2-en-1-one (2.7 mg, 12% yield) as a yellow solid. LCMS (MM-ES+APCI, Pos): m/z 603.4 (M+H).

### Example 64

### 1-(4-(8-((4-(imidazo[1,2-b]pyridazin-7-yloxy)-3-methylphenyl)amino)pyrimido[5,4-d]pyrimidin-2-yl)piperazin-1-yl)prop-2-en-1-one (compound 234)

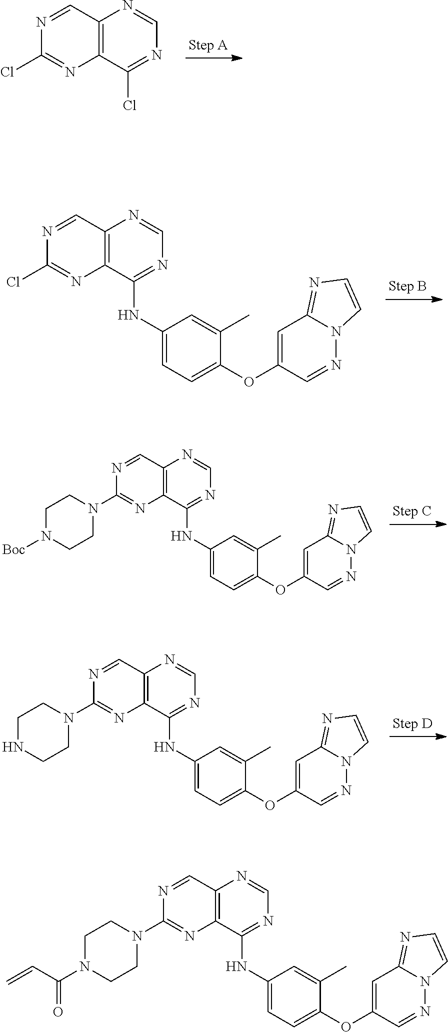 [SMILES: Clc1ncc2ncnc(Cl)c2n1 ; Cc1cc(Nc2ncnc3cnc(Cl)nc23)ccc1Oc1cnn2ccnc2c1 ; Cc1cc(Nc2ncnc3cnc(N4CCN(C(=O)OC(C)(C)C)CC4)nc23)ccc1Oc1cnn2ccnc2c1 ; Cc1cc(Nc2ncnc3cnc(N4CCNCC4)nc23)ccc1Oc1cnn2ccnc2c1 ; C=CC(=O)N1CCN(c2ncc3ncnc(Nc4ccc(Oc5cnn6ccnc6c5)c(C)c4)c3n2)CC1]

Step A. 6-chloro-N-(4-(imidazo[1,2-b]pyridazin-7-yloxy)-3-methylphenyl)pyrimido[5,4-d]pyrimidin-4-amine. A mixture of 4-imidazo[1,2-b]pyridazin-7-yloxy-3-methyl-aniline (85 mg, 0.35 mmol) and 4,6-dichloropyrimido[5,4-d]pyrimidine (78 mg, 0.39 mmol) in IPA was stirred at 30° C. for 1 h. The reaction was concentrated in vacuo to provide 6-chloro-N-(4-imidazo[1,2-b]pyridazin-7-yloxy-3-methyl-phenyl)pyrimido[5,4-d]pyrimidin-4-amine 3 (135 mg, 95% yield) as a yellow solid. LCMS (MM-ES+APCI, Pos): m/z 405.2 (M+H).

Steps B to D. 1-(4-(8-((4-(imidazo[1,2-b]pyridazin-7-yloxy)-3-methylphenyl)amino)pyrimido[5,4-d]pyrimidin-2-yl)piperazin-1-yl)prop-2-en-1-one. Prepared according to Example 55 steps A to C, substituting 6-chloro-N-(4-(imidazo[1,2-b]pyridazin-7-yloxy)-3-methylphenyl)pyrimido[5,4-d]pyrimidin-4-amine for N-[3-methyl-4-(1-methylbenzotriazol-5-yl)oxy-phenyl]-6-methylsulfinyl-pyrimido[5,4-d]pyrimidin-4-amine to afford 1-(4-(8-((4-(imidazo[1,2-b]pyridazin-7-yloxy)-3-methylphenyl)amino)pyrimido[5,4-d]pyrimidin-2-yl)piperazin-1-yl)prop-2-en-1-one (24.5 mg, 20% yield) as a yellow solid. LCMS (MM-ES+APCI, Pos): m/z 509.3 (M+H).

Compounds 234-237 in Table 18 below were synthesized according to Example 64 steps A to D.

TABLE 18
A list of compounds 234-237

| | | | MS |
|---|---|---|---|
| Cmp. | | | API |
| ID | Structure | Chemical Name | (m/z) |
| 234 | 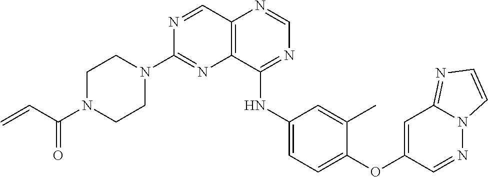 [SMILES: C=CC(=O)N1CCN(c2ncc3ncnc(Nc4ccc(Oc5cnn6ccnc6c5)c(C)c4)c3n2)CC1] | 1-(4-(8-((4-(imidazo[1,2- b]pyridazin-7-yloxy)-3- methylphenyl)amino)pyrimido[5,4- d]pyrimidin-2-yl)piperazin-1- yl)prop-2-en-1-one | 509.3 (M + H) |
| 235 | 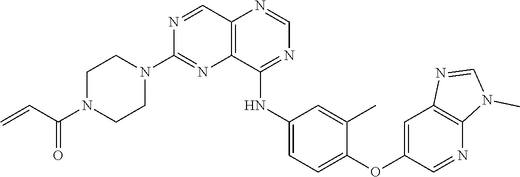 [SMILES: C=CC(=O)N1CCN(c2ncc3ncnc(Nc4ccc(Oc5cnc6c(c5)ncn6C)c(C)c4)c3n2)CC1] | 1-(4-(8-((3-methyl-4-((3-methyl-3H- imidazo[4,5-b]pyridin-6- yl)oxy)phenyl)amino)pyrimido[5,4- d]pyrimidin-2-yl)piperazin-1- yl)prop-2-en-1-one | 523.3 (M + H) |
| 236 | 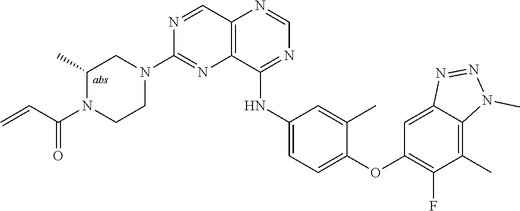 [SMILES: C=CC(=O)N1CCN(c2ncc3ncnc(Nc4ccc(Oc5cc6nnn(C)c6c(C)c5F)c(C)c4)c3n2)C[C@H]1C] | (R)-1-(4-(8-((4-((6-fluoro-1,7- dimethyl-1H-benzo[d][1,2,3]triazol- 5-yl)oxy)-3- methylphenyl)amino)pyrimido[5,4- d]pyrimidin-2-yl)-2- methylpiperazin-1-yl)prop-2-en-1- one | 569.3 (M + H) |
| 237 | 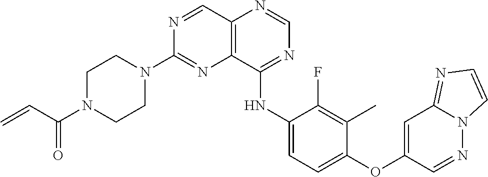 [SMILES: C=CC(=O)N1CCN(c2ncc3ncnc(Nc4ccc(Oc5cnn6ccnc6c5)c(C)c4F)c3n2)CC1] | 1-(4-(8-((2-fluoro-4-(imidazo[1,2- b]pyridazin-7-yloxy)-3- methylphenyl)amino)pyrimido[5,4- d]pyrimidin-2-yl)piperazin-1- yl)prop-2-en-1-one | 527.3 (M + H) |

### Example 65

### (R)-1′-(8-((4-([1,2,4]triazolo[1,5-a]pyridin-7-yloxy)-3-methylphenyl)amino)pyrimido[5,4-d]pyrimidin-2-yl)-3-methylene-[1,3′-bipyrrolidin]-2-one (compound 238)

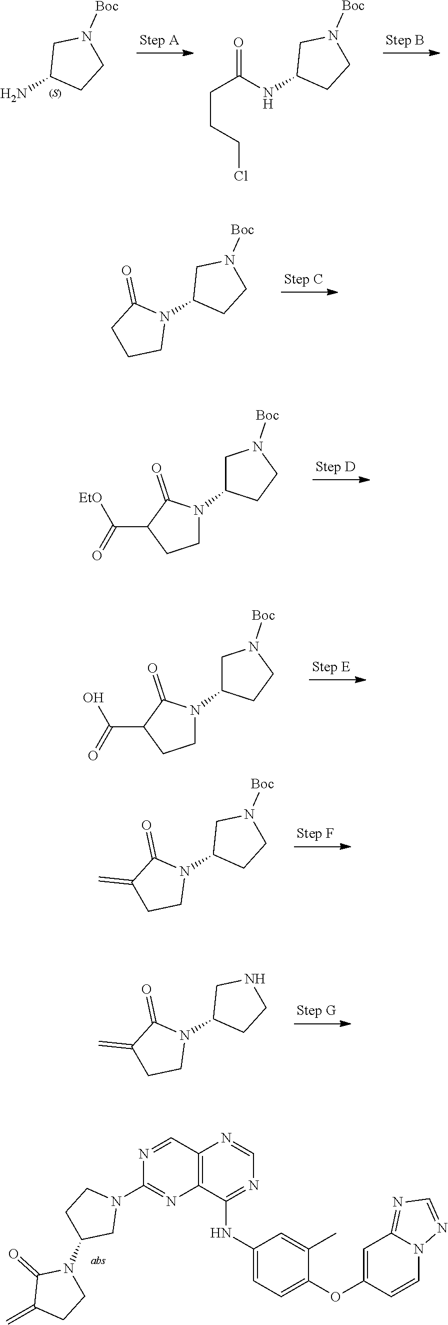

Step A. tert-butyl (S)-3-(4-chlorobutanamido)pyrrolidine-1-carboxylate. To a solution of tert-butyl (3S)-3-aminopyrrolidine-1-carboxylate (3.50 g, 18.8 mmol) in DCM (30 mL) was added TEA (2.62 mL, 18.8 mmol). The solution was cooled to 0° C. and 4-chlorobutanoyl chloride (2.10 mL, 18.8 mmol) was added. The mixture was stirred at 25° C. for 3 h. The reaction was quenched with water (120 mL) and filtered over Celite eluting with EtOAc (60 mL). The biphasic mixture was separated, and the aqueous layer was extracted with EtOAc (50 mL×3). The combined organic layers were washed with brine (60 mL), dried over anhydrous sodium sulfate, filtered, and concentrated under reduced pressure. The crude residue was purified by silica gel column chromatography (1% to 30% EtOAc/PET) to afford tert-butyl (3S)-3-(4-chlorobutanoylamino)pyrrolidine-1-carboxylate (4.70 g, 72% yield) as a white solid. LCMS (MM-ES+APCI, Pos): m/z 235.3 (M+H-^{t}Bu).

Step B. tert-butyl (S)-2-oxo-[1,3′-bipyrrolidine]-1′-carboxylate. To a solution of tert-butyl (3S)-3-(4-chlorobutanoylamino)pyrrolidine-1-carboxylate 3 (4.70 g, 13.5 mmol) in THF (80 mL) at 0° C. was added NaH (0.57 g, 14.1 mmol, 60% w/w) in portions. The mixture was stirred at 0° C. for 30 min and at 80° C. for 2 h. The reaction was quenched with sat. aq. NH₄Cl (30 mL), diluted with water (60 mL), and extracted with EtOAc (50 mL×3). The organic layers were combined, washed with water (50 mL×2) and brine (50 mL), dried over Na₂SO₄, filtered, and concentrated in vacuo. The crude residue was purified by silica gel column chromatography (1% to 20% EtOAc/PET) to provide tert-butyl (3S)-3-(2-oxopyrrolidin-1-yl)pyrrolidine-1-carboxylate (1.56 g, 46% yield) as a light yellow oil. LCMS (MM-ES+APCI, Pos): m/z 199.3 (M+H-^{t}Bu).

Step C. 1′-(tert-butyl) 3-ethyl (3'S)-2-oxo-[1,3′-bipyrrolidine]-1′,3-dicarboxylate. To a solution of tert-butyl (3S)-3-(2-oxopyrrolidin-1-yl)pyrrolidine-1-carboxylate 4 (1.40 g, 5.45 mmol) in THF (24 mL) was added LiHMDS (1 M, 10.9 mL, 10.9 mmol) at −70° C. under N₂ atmosphere. The mixture was stirred at −70° C. for 1 h, diethyl carbonate (1.32 mL, 10.9 mmol) in THF (2.4 mL) was added, and the solution was warmed to ambient temperature for 2 h. The reaction was quenched with brine (30 mL) and extracted with EtOAc (50 mL×2). The combined organic layers were dried over sodium sulfate, filtered, and concentrated in vacuo. The residue was purified by silica gel column chromatography (1% to 50% EtOAc/PET) to give ethyl 1-[(3S)-1-tert-butoxycarbonylpyrrolidin-3-yl]-2-oxo-pyrrolidine-3-carboxylate (1.25 g, 70% yield) as a yellow solid. LCMS (MM-ES+APCI, Pos): m/z 271.3 (M+H-^{t}Bu).

Step D. (3'S)-1′-(tert-butoxycarbonyl)-2-oxo-[1,3′-bipyrrolidine]-3-carboxylic acid. To a solution of ethyl 1-[(3S)-1-tert-butoxycarbonylpyrrolidin-3-yl]-2-oxo-pyrrolidine-3-carboxylate (1.00 g, 3.06 mmol) in MeOH (8 mL) and water (8 mL) was added NaOH (0.12 g, 3.06 mmol). The mixture was stirred at 25° C. for 3 h. The reaction was diluted with water (50 mL) and the suspension was filtered over Celite eluting with EtOAc. The layers were separated, and the aqueous layer was extracted with EtOAc (30 mL×3). The combined organic phases were washed with brine (50 mL), dried over anhydrous sodium sulfate, filtered, and concentrated under reduced pressure to afford 1-[(3S)-1-tert-butoxycarbonylpyrrolidin-3-yl]-2-oxo-pyrrolidine-3-carboxylic acid (575 mg, 53% yield) as a yellow solid. LCMS (MM-ES+APCI, Pos): m/z 243.3 (M+H-^{t}Bu).

Step E. tert-butyl (S)-3-methylene-2-oxo-[1,3′-bipyrrolidine]-1′-carboxylate. To a solution of 1-[(3S)-1-tert-butoxycarbonylpyrrolidin-3-yl]-2-oxo-pyrrolidine-3-carboxylic acid (600 mg, 2.01 mmol) in EtOAc (14 mL) was added (CHO)n (154 mg, 3.02 mmol). The mixture was cooled to 0° C. and Et₂NH (0.25 mL, 2.41 mmol) was added. The reaction was heated to 50° C. for 3 h. The mixture was diluted with water (80 mL) and filtered over Celite eluting with EtOAc (50 mL). The layers were partitioned and the aqueous layer was extracted with EtOAc (40 mL×3). The combined organic phases were washed with brine (80 mL), dried over anhydrous sodium sulfate, filtered, and concentrated under reduced pressure. The crude residue was purified by silica gel column chromatography (1% to 50% EtOAc/PET) to afford tert-butyl (3S)-3-(3-methylene-2-oxo-pyrrolidin-1-yl)pyrrolidine-1-carboxylate (400 mg, 72% yield) as a white solid. LCMS (MM-ES+APCI, Pos): m/z 211.3 (M+H-^{t}Bu).

Step F. (S)-3-methylene-[1,3′-bipyrrolidin]-2-one trifluoroacetate. To a solution of tert-butyl (3S)-3-(3-methylene-2-oxo-pyrrolidin-1-yl)pyrrolidine-1-carboxylate (400 mg, 1.44 mmol) in DCM (6 mL) was added TFA (1.93 mL). The reaction was stirred at 20° C. for 2 h. The mixture was concentrated under in vacuo to afford 3-methylene-1-[(3S)-pyrrolidin-3-yl]pyrrolidin-2-one trifluoroacetate (400 mg, 99% yield) as a light yellow oil. LCMS (MM-ES+APCI, Pos): m/z 167.1 (M+H).

Step G. (R)-1′-(8-((4-([1,2,4]triazolo[1,5-a]pyridin-7-yloxy)-3-methylphenyl)amino)pyrimido[5,4-d]pyrimidin-2-yl)-3-methylene-[1,3′-bipyrrolidin]-2-one. To a solution of 3-methylene-1-[(3S)-pyrrolidin-3-yl]pyrrolidin-2-one trifluoroacetate (200 mg, 0.71 mmol) in IPA (10 mL) were added DIPEA (0.93 mL, 5.35 mmol) and 6-methylsulfinyl-N-[3-methyl-4-([1,2,4]triazolo[1,5-a]pyridin-7-yloxy)phenyl]pyrimido[5,4-d]pyrimidin-4-amine (154 mg, 0.34 mmol). The reaction was heated to 80° C. and stirred for 2 h. The mixture was diluted with water (30 mL) and extracted with EtOAc (25 mL×3). The combined organic layers were washed with brine (30 mL), dried over anhydrous sodium sulfate, filtered, and concentrated under reduced pressure. The crude residue was purified by preparative HPLC (25% to 55% MeCN/0.05% aqueous NH₄OH) and lyophilized to afford 3-methylene-1-[(3R)-1-[4-[3-methyl-4-([1,2,4]triazolo[1,5-a]pyridin-7-yloxy)anilino]pyrimido[5,4-d]pyrimidin-6-yl]pyrrolidin-3-yl]pyrrolidin-2-one (12 mg, 6% yield) as a yellow solid. LCMS (MM-ES+APCI, Pos): m/z 535.2 (M+H).

Compounds 238 to 241 in Table 19 below were synthesized according to Example 65 steps A to G.

TABLE 19
A list of compounds 238-241

| | | | MS |
|---|---|---|---|
| Cmp. | | | API |
| ID | Structure | Chemical Name | (m/z) |
| 238 | 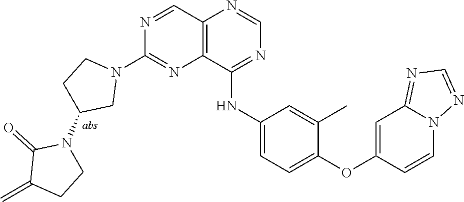 [SMILES: C=C1CCN([C@@H]2CCN(c3ncc4ncnc(Nc5ccc(Oc6ccn7ncnc7c6)c(C)c5)c4n3)C2)C1=O] | (R)-1′-(8-((4-([1,2,4]triazolo[1,5- a]pyridin-7-yloxy)-3- methylphenyl)amino)pyrimido[5,4- d]pyrimidin-2-yl)-3-methylene-[1,3′- bipyrrolidin]-2-one | 535.2 (M + H) |
| 239 | 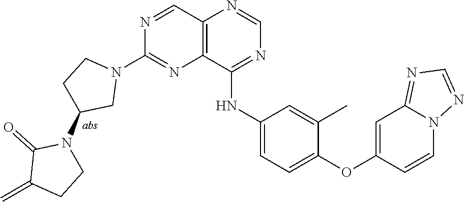 [SMILES: C=C1CCN([C@H]2CCN(c3ncc4ncnc(Nc5ccc(Oc6ccn7ncnc7c6)c(C)c5)c4n3)C2)C1=O] | (S)-1′-(8-((4-([1,2,4]triazolo[1,5- a]pyridin-7-yloxy)-3- methylphenyl)amino)pyrimido[5,4- d]pyrimidin-2-yl)-3-methylene-[1,3′- bipyrrolidin]-2-one | 535.2 (M + H) |
| 240 | 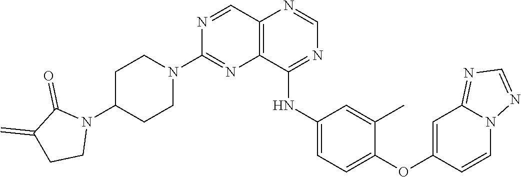 [SMILES: C=C1CCN(C2CCN(c3ncc4ncnc(Nc5ccc(Oc6ccn7ncnc7c6)c(C)c5)c4n3)CC2)C1=O] | 1-(1-(8-((4-([1,2,4]triazolo[1,5- a]pyridin-7-yloxy)-3- methylphenyl)amino)pyrimido[5,4- d]pyrimidin-2-yl)piperidin-4-yl)-3- methylenepyrrolidin-2-one | 549.4 (M + H) |
| 241 | 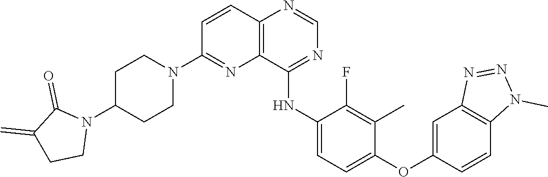 | 1-(1-(4-((2-fluoro-3-methyl-4-((1- methyl-1H-benzo[d][1,2,3]triazol-5- yl)oxy)phenyl)amino)pyrido[3,2- d]pyrimidin-6-yl)piperidin-4-yl)-3- methylenepyrrolidin-2-one | 580.5 (M + H) |

### Example 66

### 1-(6-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.0]hexan-3-yl)prop-2-en-1-one (compound 242)

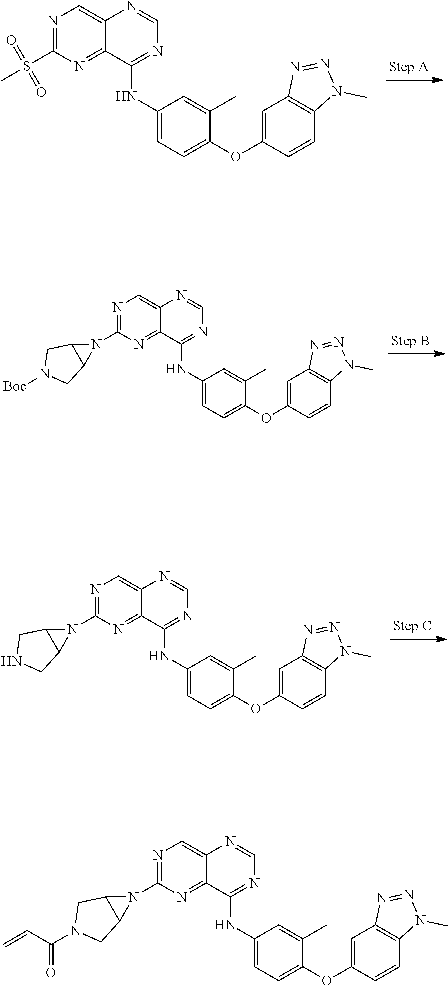

Step A. tert-butyl 6-(8-((3-methyl-4-((1-methyl-1H-benzo[d][11,2,3]triazol-5-yl)oxy)phenyl)amino)pyrimido[5,4-d]pyrimidin-2-yl)-3,6-diazabicyclo[3.1.0]hexane-3-carboxylate. To a solution of N-[3-methyl-4-(1-methylbenzotriazol-5-yl)oxy-phenyl]-6-methylsulfonyl-pyrimido[5,4-d]pyrimidin-4-amine (100 mg, 0.22 mmol) and tert-butyl 3,6-diazabicyclo[3.1.0]hexane-3-carboxylate (39.8 mg, 0.22 mmol) in DMSO (1.5 mL) was added DIPEA (75 μL, 0.43 mmol). The mixture was stirred at 80° C. for 12 h. The reaction was diluted with water (15 mL) and extracted with EtOAc (5 mL×4). The combined organic layers were washed with brine (5 mL×2), dried over anhydrous sodium sulfate, filtered, and concentrated under reduced pressure. The was purified by silica gel column chromatography (0% to 10% MeOH/DCM) to afford tert-butyl 6-[4-[3-methyl-4-(1-methylbenzotriazol-5-yl)oxy-anilino]pyrimido[5,4-d]pyrimidin-6-yl]-3,6-diazabicyclo[3.1.0]hexane-3-carboxylate (89.0 mg, 48% yield) as a yellow solid. LCMS (MM-ES+APCI, Pos): m/z 567.5 (M+H).

Step B. 6-(3,6-diazabicyclo[3.1.0]hexan-6-yl)-N-(3-methyl-4-((1-methyl-1H-benzo[d][1,2,3]triazol-5-yl)oxy)phenyl)pyrimido[5,4-d]pyrimidin-4-amine. A solution of tert-butyl 6-[4-1[3-methyl-4-(1-methylbenzotriazol-5-yl)oxy-anilino]pyrimido[5,4-d]pyrimidin-6-yl]-3,6-diazabicyclo[3.1.0]hexane-3-carboxylate (89.0 mg, 0.10 mmol) and 2,6-dimethylpyridine (72 μL, 0.62 mmol) in DCM was cooled to 0° C. TMSOTf (0.11 mL, 0.59 mmol) was added and the mixture was stirred at 0° C. for 2 h. The reaction was quenched with water (10 mL) and extracted with 10% MeOH/DCM (4 mL×3). The combined organic layers were washed with brine (4 mL×2), dried over anhydrous sodium sulfate, filtered, and concentrated under reduced pressure. The crude residue was purified by preparative TLC (10% MeOH/DCM) to afford 6-(3,6-diazabicyclo[3.1.0]hexan-6-yl)-N-[3-methyl-4-(1-methylbenzotriazol-5-yl)oxy-phenyl]pyrimido[5,4-d]pyrimidin-4-amine (40.0 mg, 71% yield) as a yellow solid. LCMS (MM-ES+APCI, Pos): m/z 467.3 (M+H).

Step C. 1-(6-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.1.0]hexan-3-yl)prop-2-en-1-one. To a solution of 6-(3,6-diazabicyclo[3.1.0]hexan-6-yl)-N-[3-methyl-4-(1-methylbenzotriazol-5-yl)oxy-phenyl]pyrimido[5,4-d]pyrimidin-4-amine (40.0 mg, 74 mol) in DCM (2 mL) was added TEA (31 μL, 221 μmol). The mixture was cooled to 0° C. and a solution of prop-2-enoyl chloride (6.0 μL, 74 μmol) in DCM (0.5 mL) was slowly added. The reaction was stirred at 0° C. for 1 h and concentrated in vacuo. MeOH (1 mL) and DMF (0.5 mL) were added to the crude residue, and the suspension was filtered. The filtrate was purified by preparative HPLC (20% to 40% MeCN/0.01% aqueous NH₄OH) and lyophilized to afford 1-[6-[4-[3-methyl-4-(1-methylbenzotriazol-5-yl)oxy-anilino]pyrimido[5,4-d]pyrimidin-6-yl]-3,6-diazabicyclo[3.1.0]hexan-3-yl]prop-2-en-1-one (7.1 mg, 18% yield) as a yellow solid. LCMS (MM-ES+APCI, Pos): m/z 521.3 (M+H).

### Example 67

### cis-1-((1r,5s)-3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.2.0]heptan-6-yl)prop-2-en-1-one (compound 243)

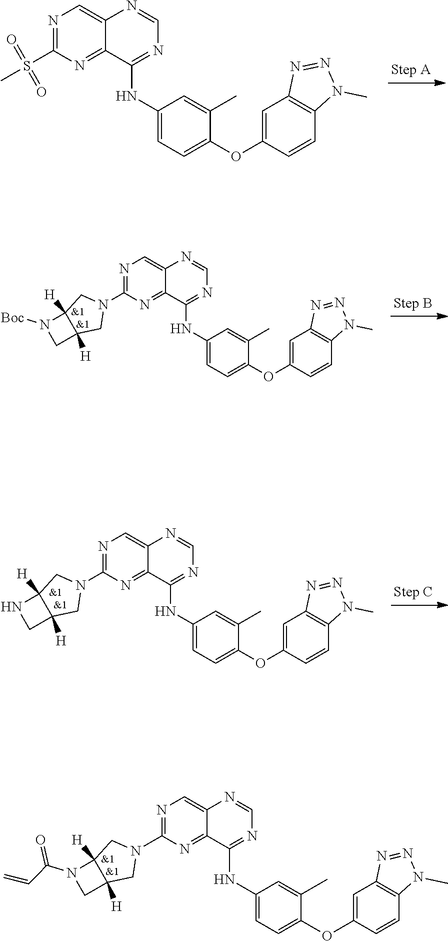

Step A. cis-tert-butyl (1r,5s)-3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.2.0]heptane-6-carboxylate. Prepared according to Example 66 step A substituting cis-tert-butyl (1r,5s)-3,6-diazabicyclo[3.2.0]heptane-6-carboxylate for and tert-butyl 3,6-diazabicyclo[3.1.0]hexane-3-carboxylate to afford cis-tert-butyl (1r,5s)-3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.2.0]heptane-6-carboxylate (100 mg, 75% yield) as a yellow solid. LCMS (MM-ES+APCI, Pos): m/z 581.3 (M+H).

Step B. cis-6-((1s,5s)-3,6-diazabicyclo[3.2.0]heptan-3-yl)-N-(3-methyl-4-((1-methyl-1H-benzo[d][1,2,3]triazol-5-yl)oxy)phenyl)pyrimido[5,4-d]pyrimidin-4-amine. To a solution of tert-butyl cis-(1r,5s)-3-[4-[3-methyl-4-(1-methylbenzotriazol-5-yl)oxy-anilino]pyrimido[5,4-d]pyrimidin-6-yl]-3,6-diazabicyclo[3.2.0]heptane-6-carboxylate (90 mg, 0.16 mmol) in DCM was added ZnBr2 (349 mg, 1.55 mmol). The reaction was stirred at 25° C. for 12 h. The mixture was added to water (10 mL), the suspension was filtered, and the filter cake was washed with EtOAc (20 mL). The solid was dried in vacuo to afford cis-6-((1s,5s)-3,6-diazabicyclo[3.2.0]heptan-3-yl)-N-(3-methyl-4-((1-methyl-1H-benzo[d][1,2,3]triazol-5-yl)oxy)phenyl)pyrimido[5,4-d]pyrimidin-4-amine (60.0 mg, 59% yield) as a yellow solid. LCMS (MM-ES+APCI, Pos): m/z 481.1 (M+H).

Step C. cis-1-((1r,5s)-3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.2.0]heptan-6-yl)prop-2-en-1-one. Prepared according to Example 66 step C, substituting cis-6-((1s,5s)-3,6-diazabicyclo[3.2.0]heptan-3-yl)-N-(3-methyl-4-((1-methyl-1H-benzo[d][1,2,3]triazol-5-yl)oxy)phenyl)pyrimido[5,4-d]pyrimidin-4-amine for 6-(3,6-diazabicyclo[3.1.0]hexan-6-yl)-N—[3-methyl-4-(1-methylbenzotriazol-5-yl)oxy-phenyl]pyrimido[5,4-d]pyrimidin-4-amine to afford cis-1-((1r,5s)-3-(8-((3-methyl-4-((1-methyl-1H-benzo[d][1,2,3]triazol-5-yl)oxy)phenyl)amino)pyrimido[5,4-d]pyrimidin-2-yl)-3,6-diazabicyclo[3.2.0]heptan-6-yl)prop-2-en-1-one (4.2 mg, 21% yield) as a yellow solid.

### Example 68

### 1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-1-one (compound 70)

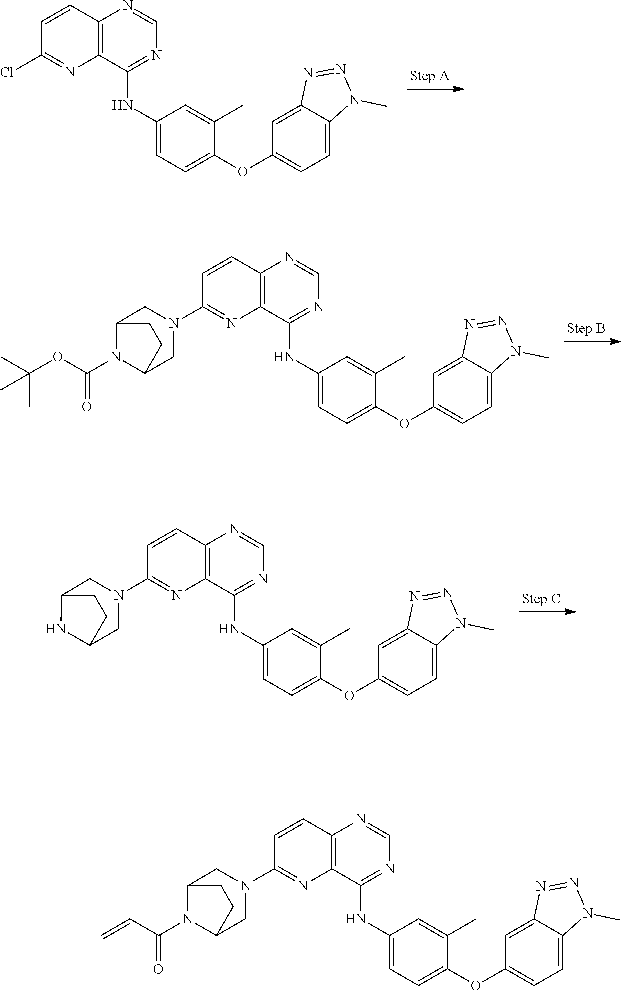

Step A. tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate. A solution of 6-chloro-N-(3-methyl-4-((1-methyl-1H-benzo[d][1,2,3]triazol-5-yl)oxy)phenyl)pyrido[3,2-d]pyrimidin-4-amine (100 mg, 0.24 mmol), tert-butyl 3,8-diazabicyclo[3.2.1]octane-8-carboxylate (102 mg, 0.48 mmol), and Cs₂CO₃ (234 mg, 3 Eq, 0.72 mmol) in DMSO (0.80 mL) was stirred at 95° C. for 16 h and at 120° C. for 15 h. The reaction was cooled to 25° C., diluted with EtOAc, and washed with water and brine. The organic phase was dried with Na₂SO₄, filtered, and concentrated in vacuo. The crude residue was purified via silica gel chromatography (0 to 20% MeOH in DCM) to obtain tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate (38.6 mg, 27%) as a yellow solid. LCMS (MM-ES+APCI, Pos): m/z 594.3 (M+H).

Step B. 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine. A solution of tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate (38.6 mg, 0.65 mmol) in DCM (0.65 mL) was added TFA (0.10 mL). The reaction was stirred for 1 h, quenched with sat. aq. NaHCO₃, and extracted with 4:1 CHCl₃:IPA. The organic phase was dried with Na₂SO₄, filtered, and concentrated in vacuo to obtain 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine (31.3 mg, 97%) as a yellow solid. LCMS (MM-ES+APCI, Pos): m/z 494.3 (M+H).

Step C. 1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-1-one. To a solution of 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine (31 mg, 0.63 mmol) and TEA (18 μL, 0.13 mmol) in DCM (0.80 mL) at −78° C. was added acryloyl chloride (5.6 μL, 0.70 mmol). The reaction was stirred at this temperature for 20 min, quenched with sat. aq. NaHCO₃ and extracted with DCM (3×). The combined organic phase was dried with Na₂SO₄, filtered, and concentrated in vacuo. The crude residue was purified via preparative HPLC (5 to 100% MeCN/0.1% aqueous TFA). The product was neutralized with sat. aq. NaHCO₃ and extracted with 20% MeOH/DCM to afford 1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-1-one (22.7 mg, 65%) as a yellow solid. LCMS (MM-ES+APCI, Pos): m/z 548.3 (M+H).

Compounds 70-82, 244-340 in Table 7 below were synthesized according to Example 68. Step A can be carried out in DMF or DMSO using inorganic or tertiary amine bases. The reaction time is shortened when using microwave heating. The Boc-deprotection (Step B) can be accomplished using HCl (4M in 1,4-dioxane or 2M in EtOAc) or TFA. For step C both DCM and THF are tolerated as solvent.

TABLE 7
A list of compounds 70-82, 244-340

| | | | MS |
|---|---|---|---|
| Cmp. | | | API |
| ID | Structure | Chemical Name | (m/z) |
| 70 | 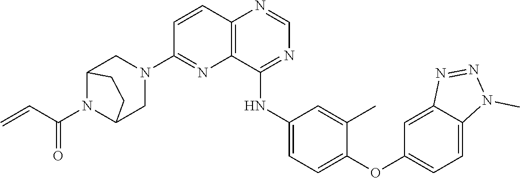 [SMILES: C=CC(=O)N1C2CCC1CN(c1ccc3ncnc(Nc4ccc(Oc5ccc6c(c5)nnn6C)c(C)c4)c3n1)C2] | 1-(3-(4-((3-methyl-4-((1-methyl- 1H-benzo[d][1,2,3]triazol-5- yl)oxy)phenyl)amino)pyrido[3,2- d]pyrimidin-6-yl)-3,8- diazabicyclo[3.2.1]octan-8-yl)prop- 2-en-1-one | 548.3 (M + H) |
| 71 | 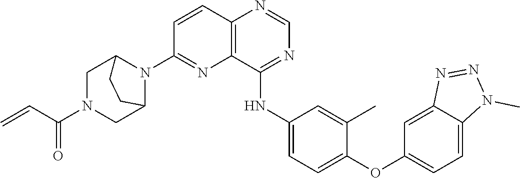 [SMILES: C=CC(=O)N1CC2CCC(C1)N2c1ccc2ncnc(Nc3ccc(Oc4ccc5c(c4)nnn5C)c(C)c3)c2n1] | 1-(8-(4-((3-methyl-4-((1-methyl- 1H-benzo[d][1,2,3]triazol-5- yl)oxy)phenyl)amino)pyrido[3,2- d]pyrimidin-6-yl)-3,8- diazabicyclo[3.2.1]octan-3-yl)prop- 2-en-1-one | 548.3 (M + H) |
| 72 | 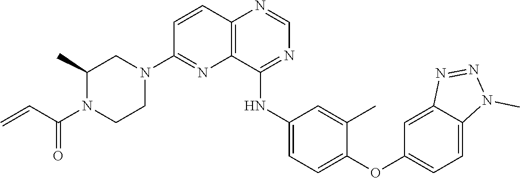 [SMILES: C=CC(=O)N1CCN(c2ccc3ncnc(Nc4ccc(Oc5ccc6c(c5)nnn6C)c(C)c4)c3n2)C[C@@H]1C] | (S)-1-(2-methyl-4-(4-((3-methyl-4- ((1-methyl-1H- benzo[d][1,2,3]triazol-5- yl)oxy)phenyl)amino)pyrido[3,2- d]pyrimidin-6-yl)piperazin-1- yl)prop-2-en-1-one | 536.3 (M + H) |
| 73 | 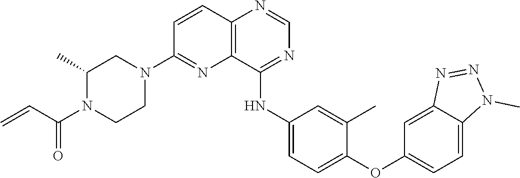 [SMILES: C=CC(=O)N1CCN(c2ccc3ncnc(Nc4ccc(Oc5ccc6c(c5)nnn6C)c(C)c4)c3n2)C[C@H]1C] | (R)-1-(2-methyl-4-(4-((3-methyl-4- ((1-methyl-1H- benzo[d][1,2,3]triazol-5- yl)oxy)phenyl)amino)pyrido[3,2- d]pyrimidin-6-yl)piperazin-1- yl)prop-2-en-1-one | 536.3 (M + H) |
| 74 | 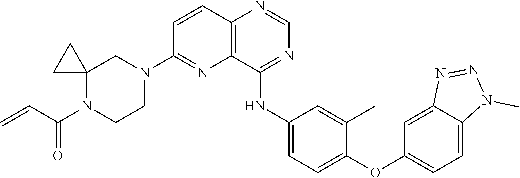 [SMILES: C=CC(=O)N1CCN(c2ccc3ncnc(Nc4ccc(Oc5ccc6c(c5)nnn6C)c(C)c4)c3n2)CC12CC2] | 1-(7-(4-((3-methyl-4-((1-methyl- 1H-benzo[d][1,2,3]triazol-5- yl)oxy)phenyl)amino)pyrido[3,2- d]pyrimidin-6-yl)-4,7- diazaspiro[2.5]octan-4-yl)prop-2- en-1-one | 548.3 (M + H) |
| 75 | 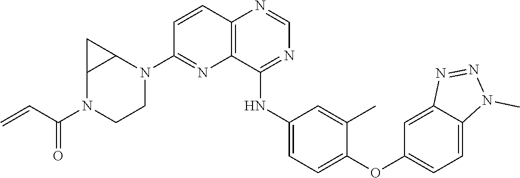 [SMILES: C=CC(=O)N1CCN(c2ccc3ncnc(Nc4ccc(Oc5ccc6c(c5)nnn6C)c(C)c4)c3n2)C2CC21] | 1-(5-(4-((3-methyl-4-((1-methyl- 1H-benzo[d][1,2,3]triazol-5- yl)oxy)phenyl)amino)pyrido[3,2- d]pyrimidin-6-yl)-2,5- diazabicyclo[4.1.0]heptan-2- yl)prop-2-en-1-one | 534.3 (M + H) |
| 76 | 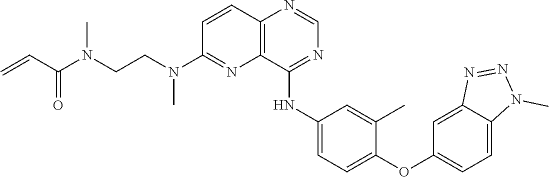 [SMILES: C=CC(=O)N(C)CCN(C)c1ccc2ncnc(Nc3ccc(Oc4ccc5c(c4)nnn5C)c(C)c3)c2n1] | N-methyl-N-(2-(methyl(4-((3- methyl-4-((1-methyl-1H- benzo[d][1,2,3]triazol-5- yl)oxy)phenyl)amino)pyrido[3,2- d]pyrimidin-6- yl)amino)ethyl)acrylamide | 524.3 (M + H) |
| 77 | 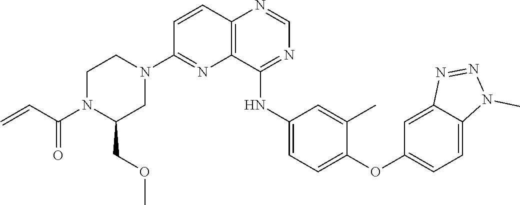 [SMILES: C=CC(=O)N1CCN(c2ccc3ncnc(Nc4ccc(Oc5ccc6c(c5)nnn6C)c(C)c4)c3n2)C[C@H]1COC] | (S)-1-(2-(methoxymethyl)-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 566.3 (M + H) |
| 78 | 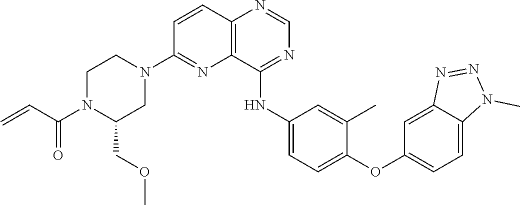 [SMILES: C=CC(=O)N1CCN(c2ccc3ncnc(Nc4ccc(Oc5ccc6c(c5)nnn6C)c(C)c4)c3n2)C[C@@H]1COC] | (R)-1-(2-(methoxymethyl)-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 566.3 (M + H) |
| 79 | 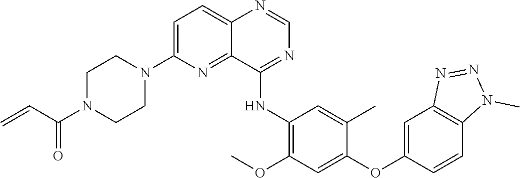 [SMILES: C=CC(=O)N1CCN(c2ccc3ncnc(Nc4cc(C)c(Oc5ccc6c(c5)nnn6C)cc4OC)c3n2)CC1] | 1-(4-(4-((2-methoxy-5-methyl-4- ((1-methyl-1H- benzo[d][1,2,3]triazol-5- yl)oxy)phenyl)amino)pyrido[3,2- d]pyrimidin-6-yl)piperazin-1- yl)prop-2-en-1-one | 552.4 (M + H) |
| 80 | 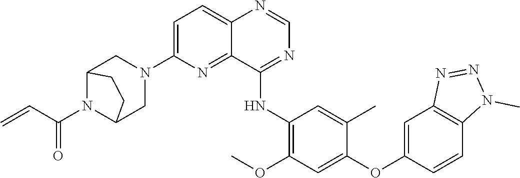 [SMILES: C=CC(=O)N1C2CCC1CN(c1ccc3ncnc(Nc4cc(C)c(Oc5ccc6c(c5)nnn6C)cc4OC)c3n1)C2] | 1-(3-(4-((2-methoxy-5-methyl-4- ((1-methyl-1H- benzo[d][1,2,3]triazol-5- yl)oxy)phenyl)amino)pyrido[3,2- d]pyrimidin-6-yl)-3,8- diazabicyclo[3.2.1]octan-8-yl)prop- 2-en-1-one | 578.4 (M + H) |
| 81 | 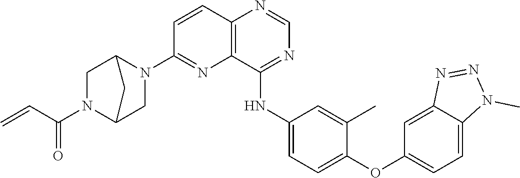 [SMILES: C=CC(=O)N1CC2CC1CN2c1ccc2ncnc(Nc3ccc(Oc4ccc5c(c4)nnn5C)c(C)c3)c2n1] | 1-(5-(4-((3-methyl-4-((1-methyl- 1H-benzo[d][1,2,3]triazol-5- yl)oxy)phenyl)amino)pyrido[3,2- d]pyrimidin-6-yl)-2,5- diazabicyclo[2.2.1]heptan-2- yl)prop-2-en-1-one | 534.3 (M + H) |
| 82 | 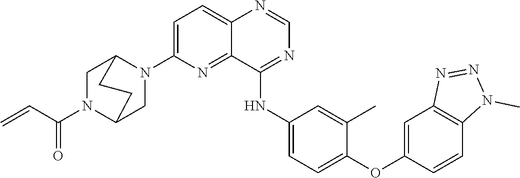 [SMILES: C=CC(=O)N1CC2CCC1CN2c1ccc2ncnc(Nc3ccc(Oc4ccc5c(c4)nnn5C)c(C)c3)c2n1] | 1-(5-(4-((3-methyl-4-((1-methyl- 1H-benzo[d][1,2,3]triazol-5- yl)oxy)phenyl)amino)pyrido[3,2- d]pyrimidin-6-yl)-2,5- diazabicyclo[2.2.2]octan-2-yl)prop- 2-en-1-one | 548.3 (M + H) |
| 244 | 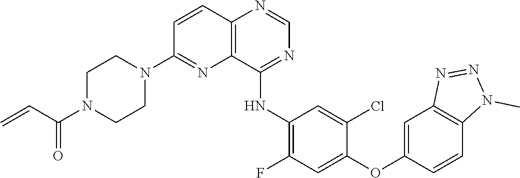 [SMILES: C=CC(=O)N1CCN(c2ccc3ncnc(Nc4cc(Cl)c(Oc5ccc6c(c5)nnn6C)cc4F)c3n2)CC1] | 1-(4-(4-((5-chloro-2-fluoro-4-((1- methyl-1H-benzo[d][1,2,3]triazol- 5-yl)oxy)phenyl)amino)pyrido[3,2- d]pyrimidin-6-yl)piperazin-1- yl)prop-2-en-1-one | 560.3 (M + H) |
| 245 | 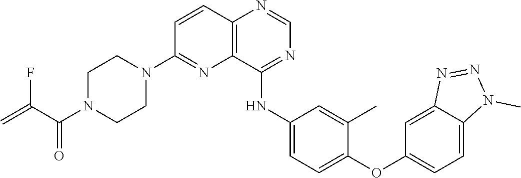 [SMILES: C=C(F)C(=O)N1CCN(c2ccc3ncnc(Nc4ccc(Oc5ccc6c(c5)nnn6C)c(C)c4)c3n2)CC1] | 2-fluoro-1-(4-(4-((3-methyl-4-((1- methyl-1H-benzo[d][1,2,3]triazol- 5-yl)oxy)phenyl)amino)pyrido[3,2- d]pyrimidin-6-yl)piperazin-1- yl)prop-2-en-1-one | 540.2 (M + H) |
| 246 | 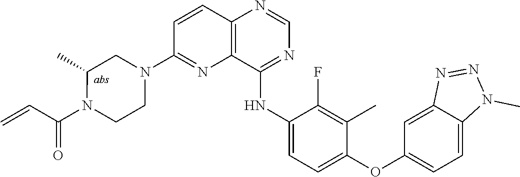 [SMILES: C=CC(=O)N1CCN(c2ccc3ncnc(Nc4ccc(Oc5ccc6c(c5)nnn6C)c(C)c4F)c3n2)C[C@H]1C] | (R)-1-(4-(4-((2-fluoro-3-methyl-4- ((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 554.2 (M + H) |
| 247 | 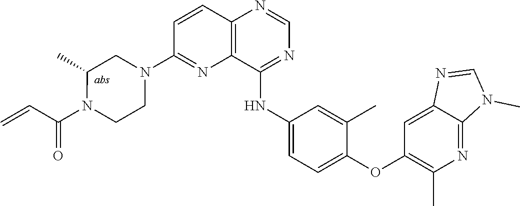 [SMILES: C=CC(=O)N1CCN(c2ccc3ncnc(Nc4ccc(Oc5cc6ncn(C)c6nc5C)c(C)c4)c3n2)C[C@H]1C] | (R)-1-(4-(4-((4-((3,5-dimethyl-3H- imidazo[4,5-b]pyridin-6-yl)oxy)-3- methylphenyl)amino)pyrido[3,2- d]pyrimidin-6-yl)-2- methylpiperazin-1-yl)prop-2-en-1- one | 550.4 (M + H) |
| 248 | 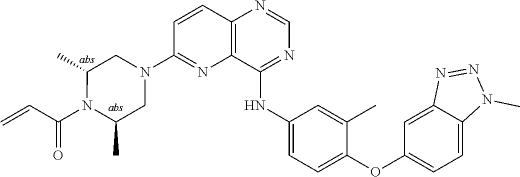 [SMILES: C=CC(=O)N1[C@H](C)CN(c2ccc3ncnc(Nc4ccc(Oc5ccc6c(c5)nnn6C)c(C)c4)c3n2)C[C@H]1C] | 1-((2R,6R)-2,6-dimethyl-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 550.3 (M + H) |
| 249 | 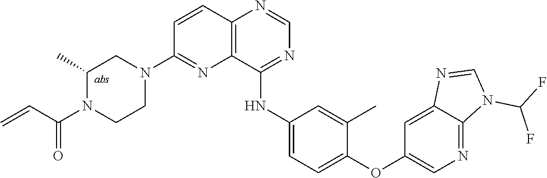 [SMILES: C=CC(=O)N1CCN(c2ccc3ncnc(Nc4ccc(Oc5cnc6c(c5)ncn6C(F)F)c(C)c4)c3n2)C[C@H]1C] | (R)-1-(4-(4-((4-((3- (difluoromethyl)-3H-imidazo[4,5- b]pyridin-6-yl)oxy)-3- methylphenyl)amino)pyrido[3,2- d]pyrimidin-6-yl)-2- methylpiperazin-1-yl)prop-2-en-1- one | 572.3 (M + H) |
| 250 | 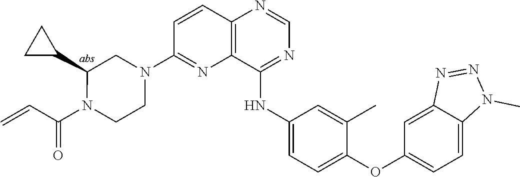 [SMILES: C=CC(=O)N1CCN(c2ccc3ncnc(Nc4ccc(Oc5ccc6c(c5)nnn6C)c(C)c4)c3n2)C[C@@H]1C1CC1] | (S)-1-(2-cyclopropyl-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 562.2 (M + H) |
| 251 | 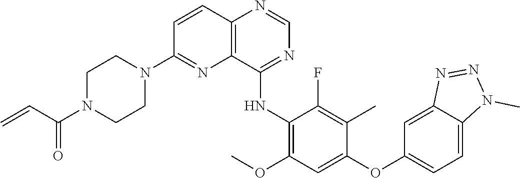 [SMILES: C=CC(=O)N1CCN(c2ccc3ncnc(Nc4c(OC)cc(Oc5ccc6c(c5)nnn6C)c(C)c4F)c3n2)CC1] | 1-(4-(4-((2-fluoro-6-methoxy-3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 570.3 (M + H) |
| 252 | 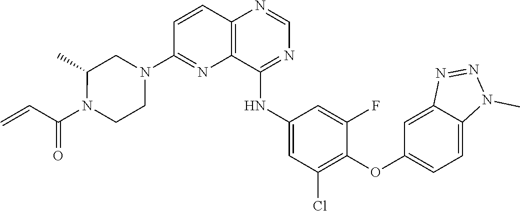 [SMILES: C=CC(=O)N1CCN(c2ccc3ncnc(Nc4cc(F)c(Oc5ccc6c(c5)nnn6C)c(Cl)c4)c3n2)C[C@H]1C] | (R)-1-(4-(4-((3-chloro-5-fluoro-4- ((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 574.2 (M + H) |
| 253 | 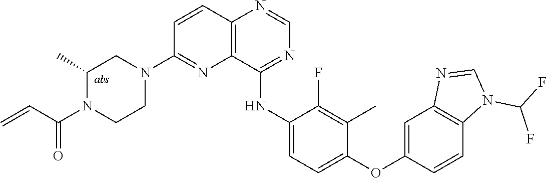 [SMILES: C=CC(=O)N1CCN(c2ccc3ncnc(Nc4ccc(Oc5ccc6c(c5)ncn6C(F)F)c(C)c4F)c3n2)C[C@H]1C] | (R)-1-(4-(4-((4-((1- (difluoromethyl)-1H- benzo[d]imidazol-5-yl)oxy)-2- fluoro-3- methylphenyl)amino)pyrido[3,2- d]pyrimidin-6-yl)-2- methylpiperazin-1-yl)prop-2-en-1- one | 589.2 (M + H) |
| 254 | 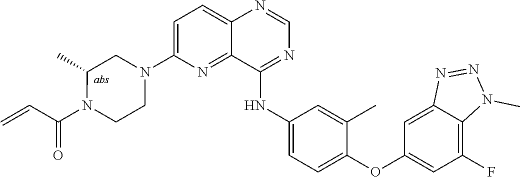 [SMILES: C=CC(=O)N1CCN(c2ccc3ncnc(Nc4ccc(Oc5cc(F)c6c(c5)nnn6C)c(C)c4)c3n2)C[C@H]1C] | (R)-1-(4-(4-((4-((7-fluoro-1- methyl-1H-benzo[d][1,2,3]triazol- 5-yl)oxy)-3- methylphenyl)amino)pyrido[3,2- d]pyrimidin-6-yl)-2- methylpiperazin-1-yl)prop-2-en-1- one | 554.3 (M + H) |
| 255 | 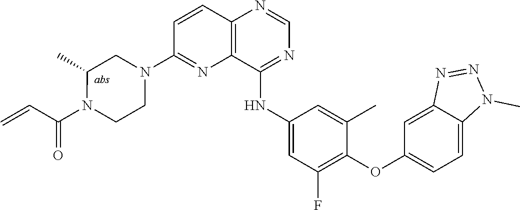 [SMILES: C=CC(=O)N1CCN(c2ccc3ncnc(Nc4cc(C)c(Oc5ccc6c(c5)nnn6C)c(F)c4)c3n2)C[C@H]1C] | (R)-1-(4-(4-((3-fluoro-5-methyl-4- ((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 554.4 (M + H) |
| 256 | 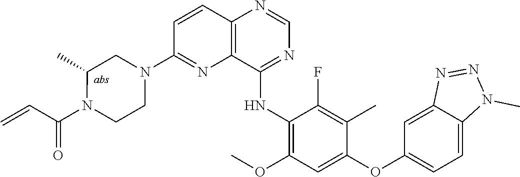 [SMILES: C=CC(=O)N1CCN(c2ccc3ncnc(Nc4c(OC)cc(Oc5ccc6c(c5)nnn6C)c(C)c4F)c3n2)C[C@H]1C] | (R)-1-(4-(4-((2-fluoro-6-methoxy- 3-methyl-4-((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 584.3 (M + H) |
| 257 | 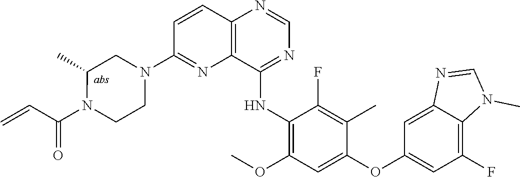 [SMILES: C=CC(=O)N1CCN(c2ccc3ncnc(Nc4c(OC)cc(Oc5cc(F)c6c(c5)ncn6C)c(C)c4F)c3n2)C[C@H]1C] | (R)-1-(4-(4-((2-fluoro-4-((7-fluoro- 1-methyl-1H-benzo[d]imidazol-5- yl)oxy)-6-methoxy-3- methylphenyl)amino)pyrido[3,2- d]pyrimidin-6-yl)-2- methylpiperazin-1-yl)prop-2-en-1- one | 601.4 (M + H) |
| 258 | 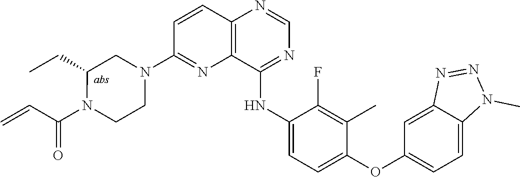 [SMILES: C=CC(=O)N1CCN(c2ccc3ncnc(Nc4ccc(Oc5ccc6c(c5)nnn6C)c(C)c4F)c3n2)C[C@H]1CC] | (R)-1-(2-ethyl-4-(4-((2-fluoro-3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 568.3 (M + H) |
| 259 | 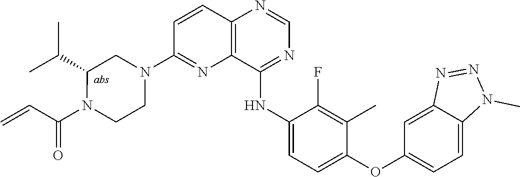 [SMILES: C=CC(=O)N1CCN(c2ccc3ncnc(Nc4ccc(Oc5ccc6c(c5)nnn6C)c(C)c4F)c3n2)C[C@H]1C(C)C] | (R)-1-(4-(4-((2-fluoro-3-methyl-4- ((1-methyl-1H- benzo[d][1,2,3]triazol-5- yl)oxy)phenyl)amino)pyrido[3,2- d]pyrimidin-6-yl)-2- isopropylpiperazin-1-yl)prop-2-en- 1-one | 582.4 (M + H) |
| 260 | 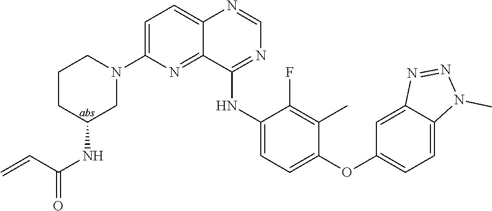 [SMILES: C=CC(=O)N[C@@H]1CCCN(c2ccc3ncnc(Nc4ccc(Oc5ccc6c(c5)nnn6C)c(C)c4F)c3n2)C1] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)piperidin-3- yl)acrylamide | 554.4 (M + H) |
| 261 | 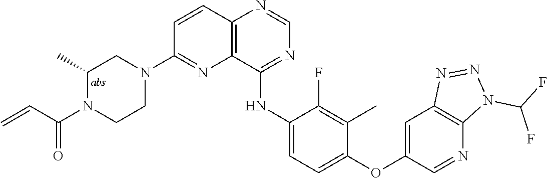 [SMILES: C=CC(=O)N1CCN(c2ccc3ncnc(Nc4ccc(Oc5cnc6c(c5)nnn6C(F)F)c(C)c4F)c3n2)C[C@H]1C] | (R)-1-(4-(4-((4-((3- (difluoromethyl)-3H-imidazo[4,5- b]pyridin-6-yl)oxy)-2-fluoro-3- methylphenyl)amino)pyrido[3,2- d]pyrimidin-6-yl)-2- methylpiperazin-1-yl)prop-2-en-1- one | 590.4 (M + H) |
| 262 | 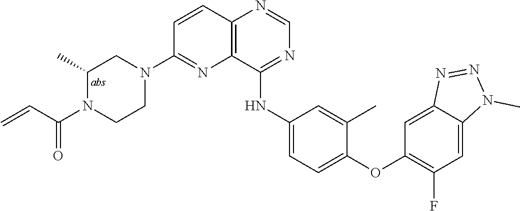 [SMILES: C=CC(=O)N1CCN(c2ccc3ncnc(Nc4ccc(Oc5cc6nnn(C)c6cc5F)c(C)c4)c3n2)C[C@H]1C] | (R)-1-(4-(4-((4-((6-fluoro-1- methyl-1H-benzo[d][1,2,3]triazol- 5-yl)oxy)-3- methylphenyl)amino)pyrido[3,2- d]pyrimidin-6-yl)-2- methylpiperazin-1-yl)prop-2-en-1- one | 554.3 (M + H) |
| 263 | 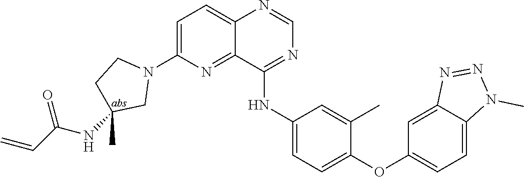 [SMILES: C=CC(=O)N[C@]1(C)CCN(c2ccc3ncnc(Nc4ccc(Oc5ccc6c(c5)nnn6C)c(C)c4)c3n2)C1] | (R)-N-(3-methyl-1-(4-((3-methyl-4- ((1-methyl-1H- benzo[d][1,2,3]triazol-5- yl)oxy)phenyl)amino)pyrido[3,2- d]pyrimidin-6-yl)pyrrolidin-3- yl)acrylamide | 536.3 (M + H) |
| 264 | 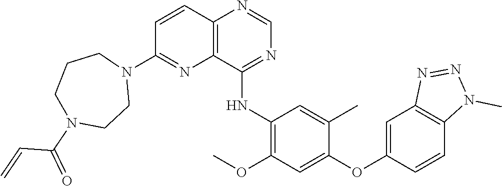 [SMILES: C=CC(=O)N1CCCN(c2ccc3ncnc(Nc4cc(C)c(Oc5ccc6c(c5)nnn6C)cc4OC)c3n2)CC1] | 1-(4-(4-((2-methoxy-5-methyl-4- ((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 566.2 (M + H) |
| 265 | 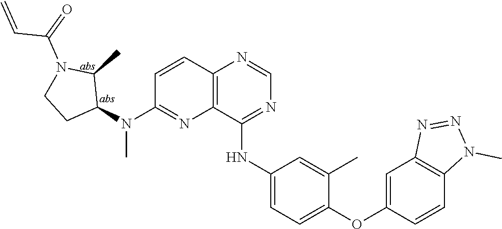 [SMILES: C=CC(=O)N1CC[C@H](N(C)c2ccc3ncnc(Nc4ccc(Oc5ccc6c(c5)nnn6C)c(C)c4)c3n2)[C@@H]1C] | 1-((2S,3S)-2-methyl-3-(methyl(4- ((3-methyl-4-((1-methyl-1H- benzo[d][1,2,3]triazol-5- yl)oxy)phenyl)amino)pyrido[3,2- d]pyrimidin-6-yl)amino)pyrrolidin- 1-yl)prop-2-en-1-one | 550.3 (M + H) |
| 266 | 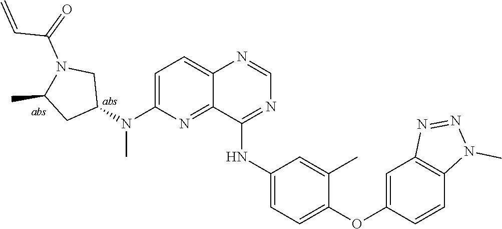 [SMILES: C=CC(=O)N1C[C@H](N(C)c2ccc3ncnc(Nc4ccc(Oc5ccc6c(c5)nnn6C)c(C)c4)c3n2)C[C@H]1C] | 1-((2R,4R)-2-methyl-4-(methyl(4- ((3-methyl-4-((1-methyl-1H- benzo[d][1,2,3]triazol-5- yl)oxy)phenyl)amino)pyrido[3,2- d]pyrimidin-6-yl)amino)pyrrolidin- 1-yl)prop-2-en-1-one | 550.3 (M + H) |
| 267 | 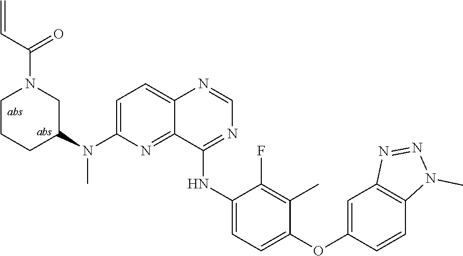 | 1-((2S,5S)-5-((4-((2-fluoro-3- methyl-4-((1-methyl-1H- benzo[d][1,2,3]triazol-5- yl)oxy)phenyl)amino)pyrido[3,2- d]pyrimidin-6-yl)(methyl)amino)- 2-methylpiperidin-1-yl)prop-2-en- 1-one | 582.3 (M + H) |
| 268 | 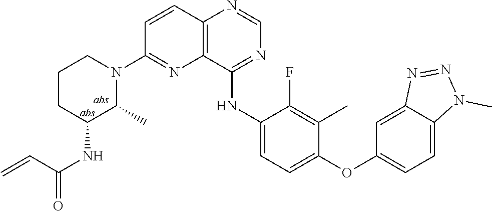 [SMILES: C=CC(=O)N[C@@H]1CCCN(c2ccc3ncnc(Nc4ccc(Oc5ccc6c(c5)nnn6C)c(C)c4F)c3n2)[C@@H]1C] | N-((2R,3R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-2- methylpiperidin-3-yl)acrylamide | 568.3 (M + H) |
| 269 | 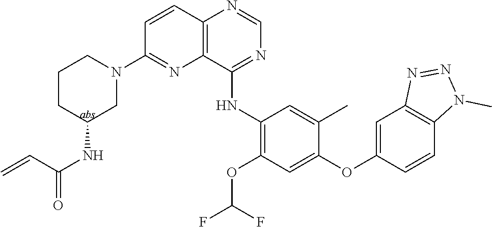 [SMILES: C=CC(=O)N[C@@H]1CCCN(c2ccc3ncnc(Nc4cc(C)c(Oc5ccc6c(c5)nnn6C)cc4OC(F)F)c3n2)C1] | (R)-N-(1-(4-((2-(difluoromethoxy)- 5-methyl-4-((1-methyl-1H- benzo[d][1,2,3]triazol-5- yl)oxy)phenyl)amino)pyrido[3,2- d]pyrimidin-6-yl)piperidin-3- yl)acrylamide | 602.2 (M + H) |
| 270 | 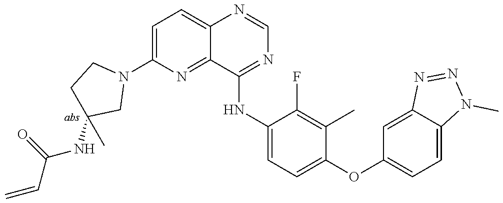 [SMILES: C=CC(=O)N[C@]1(C)CCN(c2ccc3ncnc(Nc4ccc(Oc5ccc6c(c5)nnn6C)c(C)c4F)c3n2)C1] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)-3- methylpyrrolidin-3-yl)acrylamide | 554.2 (M + H) |
| 271 | 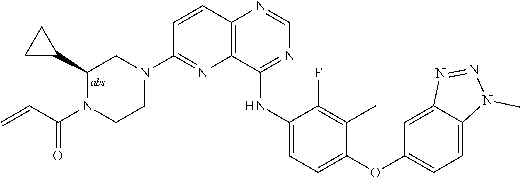 [SMILES: C=CC(=O)N1CCN(c2ccc3ncnc(Nc4ccc(Oc5ccc6c(c5)nnn6C)c(C)c4F)c3n2)C[C@@H]1C1CC1] | (S)-1-(2-cyclopropyl-4-(4-((2- fluoro-3-methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 580.2 (M + H) |
| 272 | 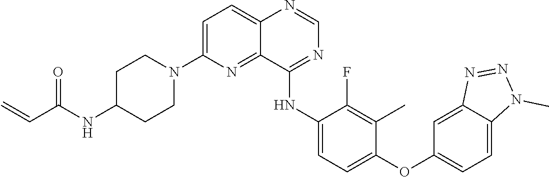 [SMILES: C=CC(=O)NC1CCN(c2ccc3ncnc(Nc4ccc(Oc5ccc6c(c5)nnn6C)c(C)c4F)c3n2)CC1] | N-(1-(4-((2-fluoro-3-methyl-4-((1- methyl-1H-benzo[d][1,2,3]triazol- 5-yl)oxy)phenyl)amino)pyrido[3,2- d]pyrimidin-6-yl)piperidin-4- yl)acrylamide | 554.2 (M + H) |
| 273 | 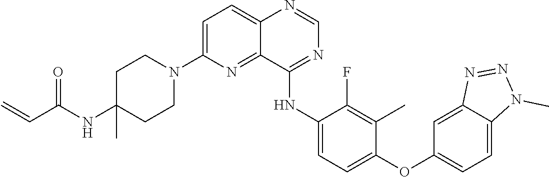 [SMILES: C=CC(=O)NC1(C)CCN(c2ccc3ncnc(Nc4ccc(Oc5ccc6c(c5)nnn6C)c(C)c4F)c3n2)CC1] | N-(1-(4-((2-fluoro-3-methyl-4-((1- methyl-1H-benzo[d][1,2,3]triazol- 5-yl)oxy)phenyl)amino)pyrido[3,2- d]pyrimidin-6-yl)-4- methylpiperidin-4-yl)acrylamide | 568.2 (M + H) |
| 274 | 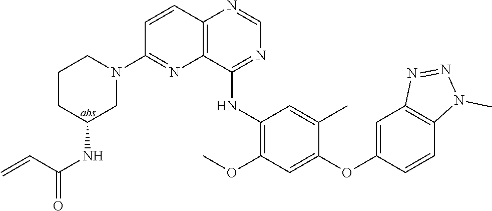 [SMILES: C=CC(=O)N[C@@H]1CCCN(c2ccc3ncnc(Nc4cc(C)c(Oc5ccc6c(c5)nnn6C)cc4OC)c3n2)C1] | (R)-N-(1-(4-((2-methoxy-5-methyl- 4-((1-methyl-1H- benzo[d][1,2,3]triazol-5- yl)oxy)phenyl)amino)pyrido[3,2- d]pyrimidin-6-yl)piperidin-3- yl)acrylamide | 566.3 (M + H) |
| 275 | 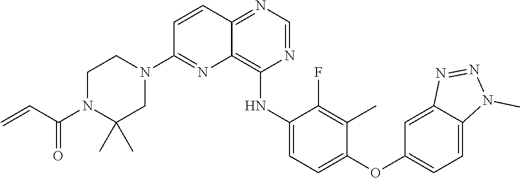 [SMILES: C=CC(=O)N1CCN(c2ccc3ncnc(Nc4ccc(Oc5ccc6c(c5)nnn6C)c(C)c4F)c3n2)CC1(C)C] | 1-(4-(4-((2-fluoro-3-methyl-4-((1- methyl-1H-benzo[d][1,2,3]triazol- 5-yl)oxy)phenyl)amino)pyrido[3,2- d]pyrimidin-6-yl)-2,2- dimethylpiperazin-1-yl)prop-2-en- 1-one | 568.2 (M + H) |
| 276 | 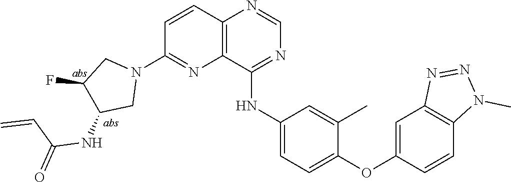 [SMILES: C=CC(=O)N[C@H]1CN(c2ccc3ncnc(Nc4ccc(Oc5ccc6c(c5)nnn6C)c(C)c4)c3n2)C[C@@H]1F] | N-((3S,4S)-4-fluoro-1-(4-((3- methyl-4-((1-methyl-1H- benzo[d][1,2,3]triazol-5- yl)oxy)phenyl)amino)pyrido[3,2- d]pyrimidin-6-yl)pyrrolidin-3- yl)acrylamide | 540.3 (M + H) |
| 277 | 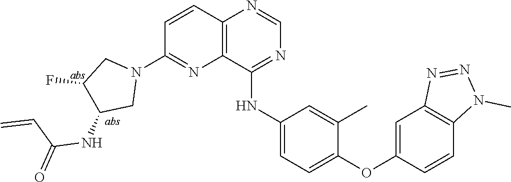 [SMILES: C=CC(=O)N[C@H]1CN(c2ccc3ncnc(Nc4ccc(Oc5ccc6c(c5)nnn6C)c(C)c4)c3n2)C[C@H]1F] | N-((3S,4R)-4-fluoro-1-(4-((3- methyl-4-((1-methyl-1H- benzo[d][1,2,3]triazol-5- yl)oxy)phenyl)amino)pyrido[3,2- d]pyrimidin-6-yl)pyrrolidin-3- yl)acrylamide | 540.3 (M + H) |
| 278 | 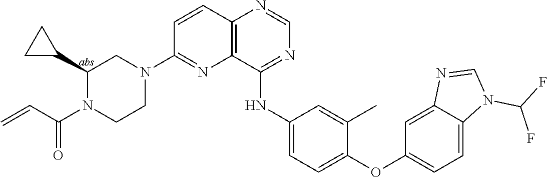 [SMILES: C=CC(=O)N1CCN(c2ccc3ncnc(Nc4ccc(Oc5ccc6c(c5)ncn6C(F)F)c(C)c4)c3n2)C[C@@H]1C1CC1] | (S)-1-(2-cyclopropyl-4-(4-((4-((1- (difluoromethyl)-1H- benzo[d]imidazol-5-yl)oxy)-3- methylphenyl)amino)pyrido[3,2- d]pyrimidin-6-yl)piperazin-1- yl)prop-2-en-1-one | 597.5 (M + H) |
| 279 | 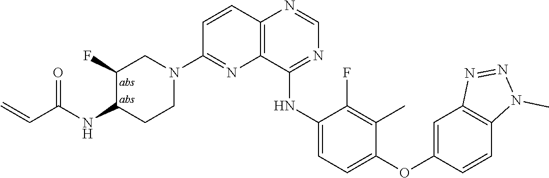 | N-((3S,4R)-3-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-4- yl)acrylamide | 572.3 (M + H) |
| 280 | 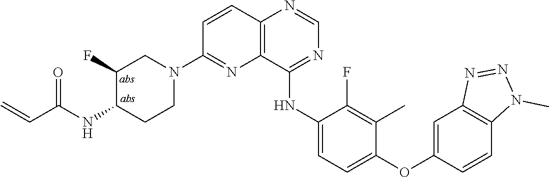 [SMILES: C=CC(=O)N[C@H]1CCN(c2ccc3ncnc(Nc4ccc(Oc5ccc6c(c5)nnn6C)c(C)c4F)c3n2)C[C@@H]1F] | N-((3S,4S)-3-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-4- yl)acrylamide | 572.3 (M + H) |
| 281 | 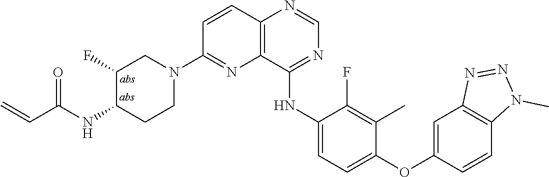 [SMILES: C=CC(=O)N[C@H]1CCN(c2ccc3ncnc(Nc4ccc(Oc5ccc6c(c5)nnn6C)c(C)c4F)c3n2)C[C@H]1F] | N-((3R,4S)-3-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-4- yl)acrylamide | 572.1 (M + H) |
| 282 | 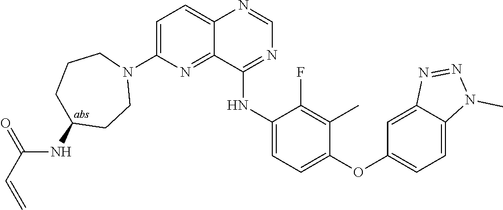 [SMILES: C=CC(=O)N[C@H]1CCCN(c2ccc3ncnc(Nc4ccc(Oc5ccc6c(c5)nnn6C)c(C)c4F)c3n2)CC1] | (S)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)azepan-4- yl)acrylamide | 568.2 (M + H) |
| 283 | 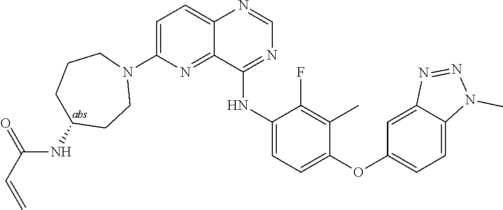 [SMILES: C=CC(=O)N[C@@H]1CCCN(c2ccc3ncnc(Nc4ccc(Oc5ccc6c(c5)nnn6C)c(C)c4F)c3n2)CC1] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)azepan-4- yl)acrylamide | 568.2 (M + H) |
| 284 | 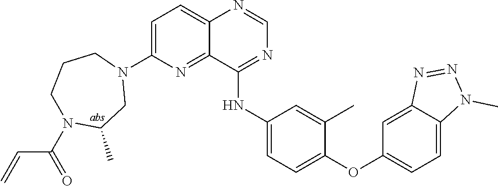 [SMILES: C=CC(=O)N1CCCN(c2ccc3ncnc(Nc4ccc(Oc5ccc6c(c5)nnn6C)c(C)c4)c3n2)C[C@@H]1C] | (S)-1-(2-methyl-4-(4-((3-methyl-4- ((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 550.2 (M + H) |
| 285 | 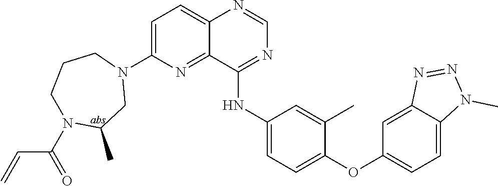 [SMILES: C=CC(=O)N1CCCN(c2ccc3ncnc(Nc4ccc(Oc5ccc6c(c5)nnn6C)c(C)c4)c3n2)C[C@H]1C] | (R)-1-(2-methyl-4-(4-((3-methyl-4- ((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 550.5 (M + H) |
| 286 | 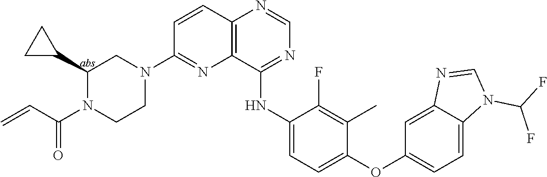 [SMILES: C=CC(=O)N1CCN(c2ccc3ncnc(Nc4ccc(Oc5ccc6c(c5)ncn6C(F)F)c(C)c4F)c3n2)C[C@@H]1C1CC1] | (S)-1-(2-cyclopropyl-4-(4-((4-((1- (difluoromethyl)-1H- benzo[d]imidazol-5-yl)oxy)-2- fluoro-3- methylphenyl)amino)pyrido[3,2- d]pyrimidin-6-yl)piperazin-1- yl)prop-2-en-1-one | 615.2 (M + H) |
| 287 | 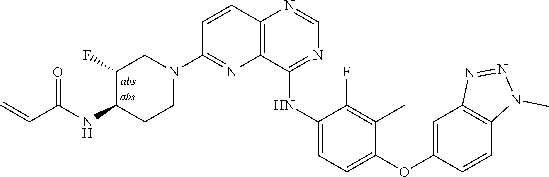 [SMILES: C=CC(=O)N[C@@H]1CCN(c2ccc3ncnc(Nc4ccc(Oc5ccc6c(c5)nnn6C)c(C)c4F)c3n2)C[C@H]1F] | N-((3R,4R)-3-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-4- yl)acrylamide | 572.2 (M + H) |
| 288 | 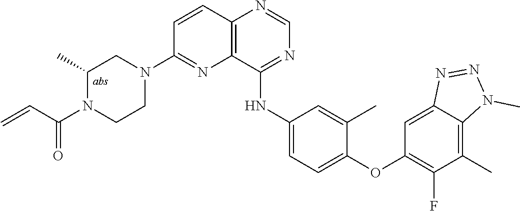 [SMILES: C=CC(=O)N1CCN(c2ccc3ncnc(Nc4ccc(Oc5cc6nnn(C)c6c(C)c5F)c(C)c4)c3n2)C[C@H]1C] | (R)-1-(4-(4-((4-((6-fluoro-1,7- dimethyl-1H- benzo[d][1,2,3]triazol-5-yl)oxy)-3- methylphenyl)amino)pyrido[3,2- d]pyrimidin-6-yl)-2- methylpiperazin-1-yl)prop-2-en-1- one | 568.0 (M + H) |
| 289 | 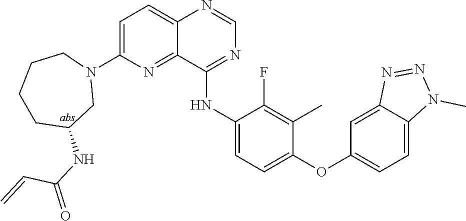 [SMILES: C=CC(=O)N[C@@H]1CCCCN(c2ccc3ncnc(Nc4ccc(Oc5ccc6c(c5)nnn6C)c(C)c4F)c3n2)C1] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)azepan-3- yl)acrylamide | 568.3 (M + H) |
| 290 | 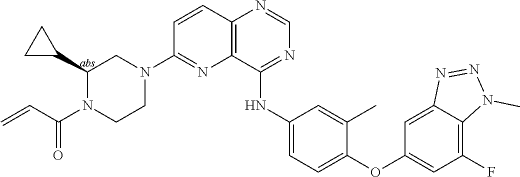 [SMILES: C=CC(=O)N1CCN(c2ccc3ncnc(Nc4ccc(Oc5cc(F)c6c(c5)nnn6C)c(C)c4)c3n2)C[C@@H]1C1CC1] | (S)-1-(2-cyclopropyl-4-(4-((4-((7- fluoro-1-methyl-1H- benzo[d][1,2,3]triazol-5-yl)oxy)-3- methylphenyl)amino)pyrido[3,2- d]pyrimidin-6-yl)piperazin-1- yl)prop-2-en-1-one | 580.2 (M + H) |
| 291 | 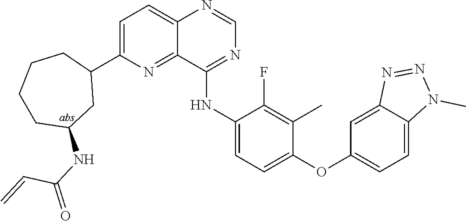 [SMILES: C=CC(=O)N[C@H]1CCCCC(c2ccc3ncnc(Nc4ccc(Oc5ccc6c(c5)nnn6C)c(C)c4F)c3n2)C1] | (S)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)azepan-3- yl)acrylamide | 568.3 (M + H) |
| 292 | 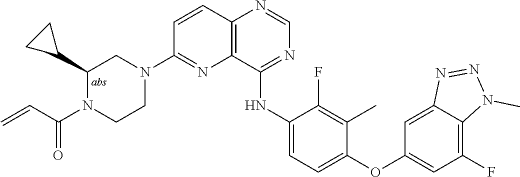 [SMILES: C=CC(=O)N1CCN(c2ccc3ncnc(Nc4ccc(Oc5cc(F)c6c(c5)nnn6C)c(C)c4F)c3n2)C[C@@H]1C1CC1] | (S)-1-(2-cyclopropyl-4-(4-((2- fluoro-4-((7-fluoro-1-methyl-1H- benzo[d][1,2,3]triazol-5-yl)oxy)-3- methylphenyl)amino)pyrido[3,2- d]pyrimidin-6-yl)piperazin-1- yl)prop-2-en-1-one | 598.2 (M + H) |
| 293 | 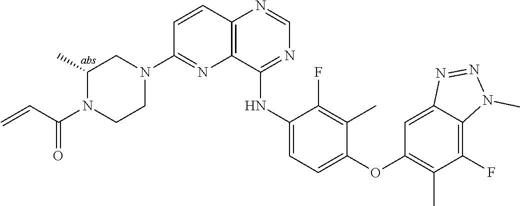 [SMILES: C=CC(=O)N1CCN(c2ccc3ncnc(Nc4ccc(Oc5cc6nnn(C)c6c(F)c5C)c(C)c4F)c3n2)C[C@H]1C] | (R)-1-(4-(4-((2-fluoro-4-((7-fluoro- 1,6-dimethyl-1H- benzo[d][1,2,3]triazol-5-yl)oxy)-3- methylphenyl)amino)pyrido[3,2- d]pyrimidin-6-yl)-2- methylpiperazin-1-yl)prop-2-en-1- one | 586.2 (M + H) |
| 294 | 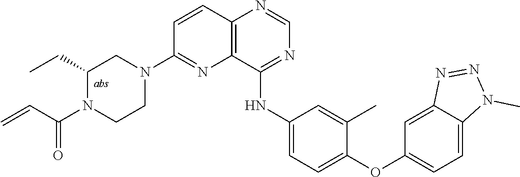 [SMILES: C=CC(=O)N1CCN(c2ccc3ncnc(Nc4ccc(Oc5ccc6c(c5)nnn6C)c(C)c4)c3n2)C[C@H]1CC] | (R)-1-(2-ethyl-4-(4-((3-methyl-4- ((1-methyl-1H- benzo[d][1,2,3]triazol-5- yl)oxy)phenyl)amino)pyrido[3,2- d]pyrimidin-6-yl)piperazin-1- yl)prop-2-en-1-one | 550.4 (M + H) |
| 295 | 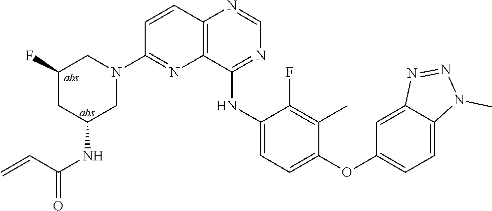 [SMILES: C=CC(=O)N[C@@H]1C[C@@H](F)CN(c2ccc3ncnc(Nc4ccc(Oc5ccc6c(c5)nnn6C)c(C)c4F)c3n2)C1] | N-((3R,5R)-5-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-3- yl)acrylamide | 572.3 (M + H) |
| 296 | 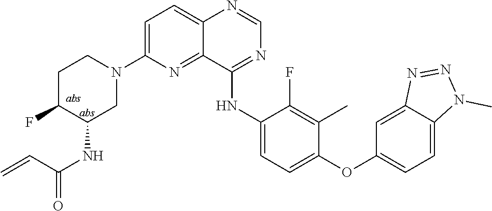 [SMILES: C=CC(=O)N[C@H]1CN(c2ccc3ncnc(Nc4ccc(Oc5ccc6c(c5)nnn6C)c(C)c4F)c3n2)CC[C@@H]1F] | N-((3S,4S)-4-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-3- yl)acrylamide | 572.3 (M + H) |
| 297 | 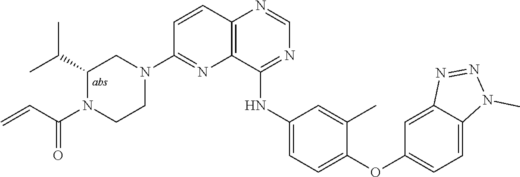 [SMILES: C=CC(=O)N1CCN(c2ccc3ncnc(Nc4ccc(Oc5ccc6c(c5)nnn6C)c(C)c4)c3n2)C[C@H]1C(C)C] | (R)-1-(2-isopropyl-4-(4-((3-methyl- 4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 564.2 (M + H) |
| 298 | 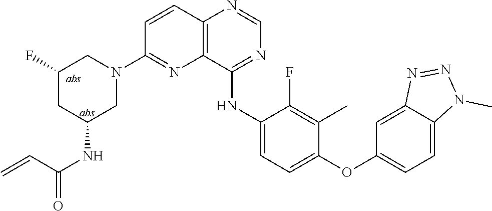 [SMILES: C=CC(=O)N[C@@H]1C[C@H](F)CN(c2ccc3ncnc(Nc4ccc(Oc5ccc6c(c5)nnn6C)c(C)c4F)c3n2)C1] | N-((3R,5S)-5-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)piperidin-3- yl)acrylamide | 572.1 (M + H) |
| 299 | 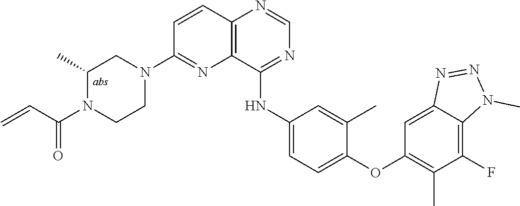 [SMILES: C=CC(=O)N1CCN(c2ccc3ncnc(Nc4ccc(Oc5cc6nnn(C)c6c(F)c5C)c(C)c4)c3n2)C[C@H]1C] | (R)-1-(4-(4-((4-((7-fluoro-1,6- dimethyl-1H- benzo[d][1,2,3]triazol-5-yl)oxy)-3- methylphenyl)amino)pyrido[3,2- d]pyrimidin-6-yl)-2- methylpiperazin-1-yl)prop-2-en-1- one | 568.2 (M + H) |
| 300 | 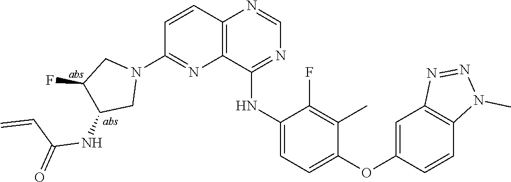 [SMILES: C=CC(=O)N[C@H]1CN(c2ccc3ncnc(Nc4ccc(Oc5ccc6c(c5)nnn6C)c(C)c4F)c3n2)C[C@@H]1F] | N-((3S,4S)-4-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)pyrrolidin-3- yl)acrylamide | 558.3 (M + H) |
| 301 | 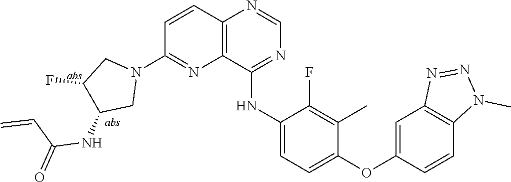 [SMILES: C=CC(=O)N[C@H]1CN(c2ccc3ncnc(Nc4ccc(Oc5ccc6c(c5)nnn6C)c(C)c4F)c3n2)C[C@H]1F] | N-((3S,4R)-4-fluoro-1-(4-((2- fluoro-3-methyl-4-((1-methyl-1H- benzo[d][1,2,3]triazol-5- yl)oxy)phenyl)amino)pyrido[3,2- d]pyrimidin-6-yl)pyrrolidin-3- yl)acrylamide | 558.3 (M + H) |
| 302 | 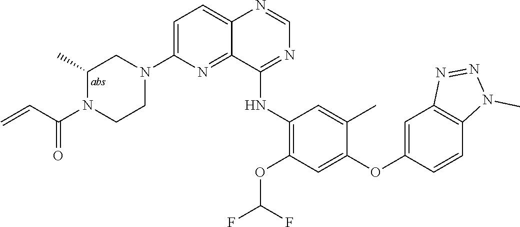 [SMILES: C=CC(=O)N1CCN(c2ccc3ncnc(Nc4cc(C)c(Oc5ccc6c(c5)nnn6C)cc4OC(F)F)c3n2)C[C@H]1C] | (R)-1-(4-(4-((2-(difluoromethoxy)- 5-methyl-4-((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 602.4 (M + H) |
| 303 | 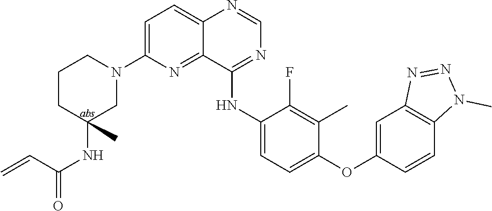 [SMILES: C=CC(=O)N[C@]1(C)CCCN(c2ccc3ncnc(Nc4ccc(Oc5ccc6c(c5)nnn6C)c(C)c4F)c3n2)C1] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)-3- methylpiperidin-3-yl)acrylamide | 568.3 (M + H) |
| 304 | 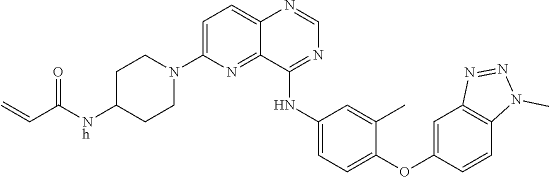 | N-(1-(4-((3-methyl-4-((1-methyl- 1H-benzo[d][1,2,3]triazol-5- yl)oxy)phenyl)amino)pyrido[3,2- d]pyrimidin-6-yl)piperidin-4- yl)acrylamide | 536.3 (M + H) |
| 305 | 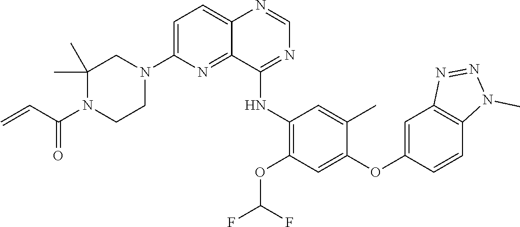 [SMILES: C=CC(=O)N1CCN(c2ccc3ncnc(Nc4cc(C)c(Oc5ccc6c(c5)nnn6C)cc4OC(F)F)c3n2)CC1(C)C] | 1-(4-(4-((2-(difluoromethoxy)-5- methyl-4-((1-methyl-1H- benzo[d][1,2,3]triazol-5- yl)oxy)phenyl)amino)pyrido[3,2- d]pyrimidin-6-yl)-2,2- dimethylpiperazin-1-yl)prop-2-en- 1-one | 616.3 (M + H) |
| 306 | 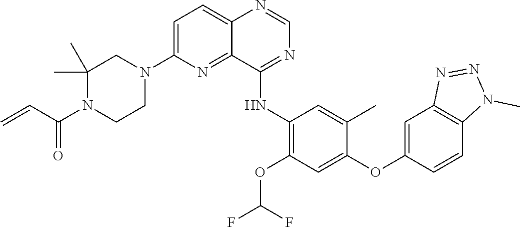 | 1-(2,2-dimethyl-4-(4-((5-methyl-4- ((1-methyl-1H- benzo[d][1,2,3]triazol-5-yl)oxy)-2- (trifluoromethoxy)phenyl)amino)py- rido[3,2-d]pyrimidin-6- yl)piperazin-1-yl)prop-2-en-1-one | 634.3 (M +H) |
| 307 | 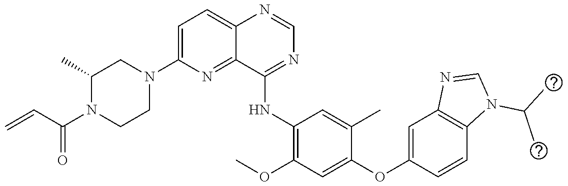 | (R)-1-(4-(4-((4-((1- (difluoromethyl)-1H- benzo[d]imidazol-5-yl)oxy)-2- methoxy-5- methylphenyl)amino)pyrido[3,2- d]pyrimidin-6-yl)-2- methylpiperazin-1-yl)prop-2-en-1- one | 601.3 (M + H) |
| 308 | 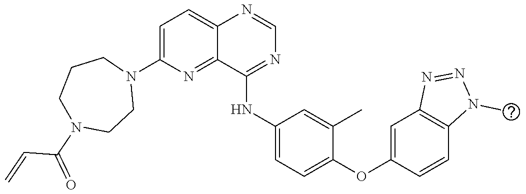 [SMILES: C=CC(=O)N1CCCN(c2ccc3ncnc(Nc4ccc(Oc5ccc6c(c5)nnn6C)c(C)c4)c3n2)CC1] | 1-(4-(4-((3-methyl-4-((1-methyl- 1H-benzo[d][1,2,3]triazol-5- yl)oxy)phenyl)amino)pyrido[3,2- d]pyrimidin-6-yl)-1,4-diazepan-1- yl)prop-2-en-1-one | 536.3 (M + H) |
| 309 | 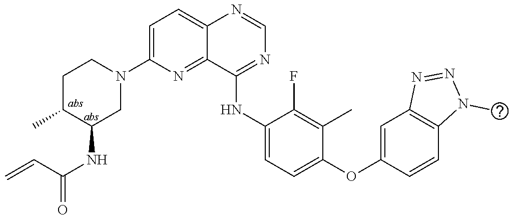 [SMILES: C=CC(=O)N[C@@H]1CN(c2ccc3ncnc(Nc4ccc(Oc5ccc6c(c5)nnn6C)c(C)c4F)c3n2)CC[C@H]1C] | N-((3S,4R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-4- methylpiperidin-3-y1)acrylamide | 568.3 (M + H) |
| 310 | 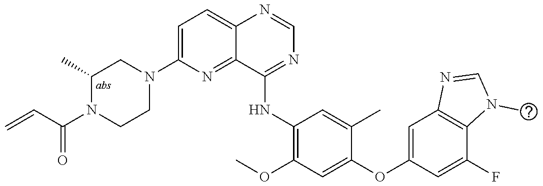 [SMILES: C=CC(=O)N1CCN(c2ccc3ncnc(Nc4cc(C)c(Oc5cc(F)c6c(c5)ncn6C)cc4OC)c3n2)C[C@H]1C] | (R)-1-(4-(4-((4-((7-fluoro-1- methyl-1H-benzo[d]imidazol-5- yl)oxy)-2-methoxy-5- methylphenyl)amino)pyrido[3,2- d]pyrimidin-6-yl)-2- methylpiperazin-1-yl)prop-2-en-1- one | 583.3 (M + H) |
| 311 | 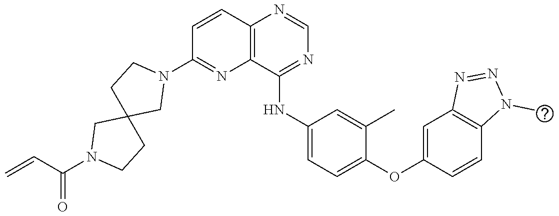 [SMILES: C=CC(=O)N1CCC2(CCN(c3ccc4ncnc(Nc5ccc(Oc6ccc7c(c6)nnn7C)c(C)c5)c4n3)C2)C1] | 1-(7-(4-((3-methyl-4-((1-methyl- 1H-benzo[d][1,2,3]triazol-5- yl)oxy)phenyl)amino)pyrido[3,2- d]pyrimidin-6-yl)-2,7- diazaspiro[4.4]nonan-2-yl)prop-2- en-1-one | 562.3 (M + H) |
| 312 | 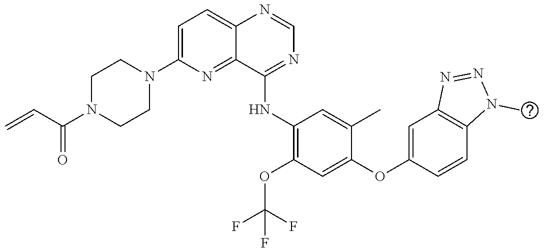 [SMILES: C=CC(=O)N1CCN(c2ccc3ncnc(Nc4cc(C)c(Oc5ccc6c(c5)nnn6C)cc4OC(F)(F)F)c3n2)CC1] | 1-(4-(4-((5-methyl-4-((1-methyl- 1H-benzo[d][1,2,3]triazol-5- yl)oxy)-2- (trifluoromethoxy)phenyl)amino)py- rido[3,2-d]pyrimidin-6- yl)piperazin-1-yl)prop-2-en-1-one | 606.2 (M +H) |
| 313 | 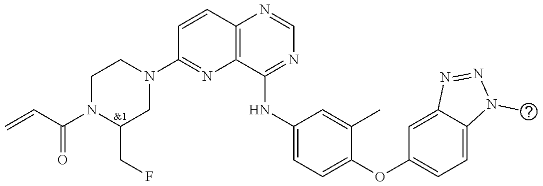 [SMILES: C=CC(=O)N1CCN(c2ccc3ncnc(Nc4ccc(Oc5ccc6c(c5)nnn6C)c(C)c4)c3n2)CC1CF] | 1-(2-(fluoromethyl)-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 554.3 (M + H) |
| 314 | 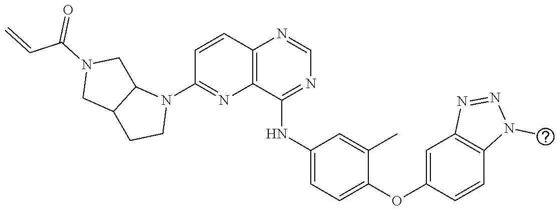 [SMILES: C=CC(=O)N1CC2CCN(c3ccc4ncnc(Nc5ccc(Oc6ccc7c(c6)nnn7C)c(C)c5)c4n3)C2C1] | 1-(1-(4-((3-methyl-4-((1-methyl- 1H-benzo[d][1,2,3]triazol-5- yl)oxy)phenyl)amino)pyrido[3,2- d]pyrimidin-6- yl)hexahydropyrrolo[3,4-b]pyrrol- 5(1H)-y1)prop-2-en-1-one | 548.2 (M + H) |
| 315 | 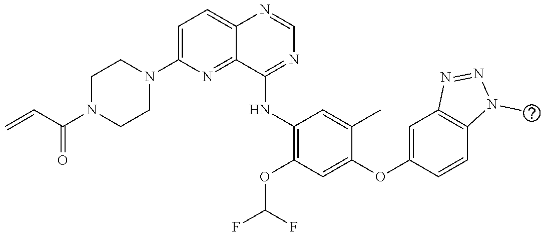 [SMILES: C=CC(=O)N1CCN(c2ccc3ncnc(Nc4cc(C)c(Oc5ccc6c(c5)nnn6C)cc4OC(F)F)c3n2)CC1] | 1-(4-(4-((2-(difluoromethoxy)-5- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 588.3 (M + H) |
| 316 | 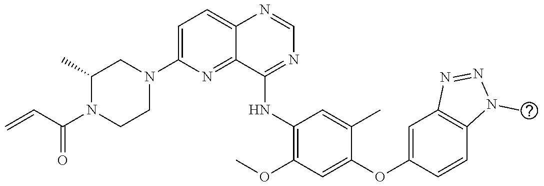 [SMILES: C=CC(=O)N1CCN(c2ccc3ncnc(Nc4cc(C)c(Oc5ccc6c(c5)nnn6C)cc4OC)c3n2)C[C@H]1C] | (R)-1-(4-(4-((2-methoxy-5-methyl- 4-((1-methyl-1H- benzo[d][1,2,3]triazol-5- yl)oxy)phenyl)amino)pyrido[3,2- d]pyrimidin-6-y1)-2- methylpiperazin-1-yl)prop-2-en-1- one | 566.3 (M + H) |
| 317 | 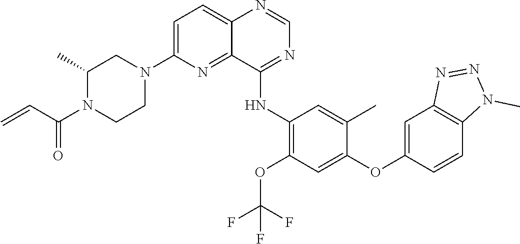 [SMILES: C=CC(=O)N1CCN(c2ccc3ncnc(Nc4cc(C)c(Oc5ccc6c(c5)nnn6C)cc4OC(F)(F)F)c3n2)C[C@H]1C] | (R)-1-(2-methyl-4-(4-((5-methyl-4- ((1-methyl-1H- benzo[d][1,2,3]triazol-5-yl)oxy)-2- (trifluoromethoxy)phenyl)amino)py- rido[3,2-d]pyrimidin-6- yl)piperazin-1-yl)prop-2-en-1-one | 620.2 (M + H) |
| 318 | 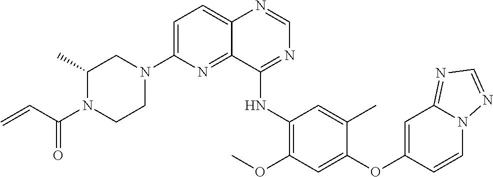 [SMILES: C=CC(=O)N1CCN(c2ccc3ncnc(Nc4cc(C)c(Oc5ccn6ncnc6c5)cc4OC)c3n2)C[C@H]1C] | (R)-1-(4-(4-((4-([1,2,4]triazolo[1,5- a]pyridin-7-yloxy)-2-methoxy-5- methylphenyl)amino)pyrido[3,2- d]pyrimidin-6-yl)-2- methylpiperazin-1-yl)prop-2-en-1- one | 552.3 (M + H) |
| 319 | 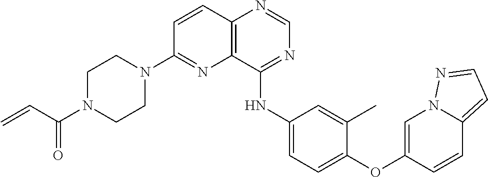 [SMILES: C=CC(=O)N1CCN(c2ccc3ncnc(Nc4ccc(Oc5ccc6ccnn6c5)c(C)c4)c3n2)CC1] | 1-(4-(4-((3-methyl-4-(pyrazolo[1,5- a]pyrimidin-6- yloxy)phenyl)amino)pyrido[3,2- d]pyrimidin-6-yl)piperazin-1- yl)prop-2-en-1-one | 508.2 (M + H) |
| 320 | 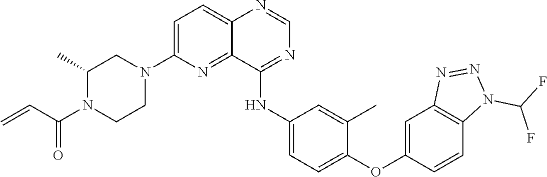 [SMILES: C=CC(=O)N1CCN(c2ccc3ncnc(Nc4ccc(Oc5ccc6c(c5)nnn6C(F)F)c(C)c4)c3n2)C[C@H]1C] | (R)-1-(4-(4-((4-((1- (difluoromethyl)-1H- benzo[d][1,2,3]triazol-5-yl)oxy)-3- methylphenyl)amino)pyrido[3,2- d]pyrimidin-6-yl)-2- methylpiperazin-1-yl)prop-2-en-1- one | 572.3 (M + H) |
| 321 | 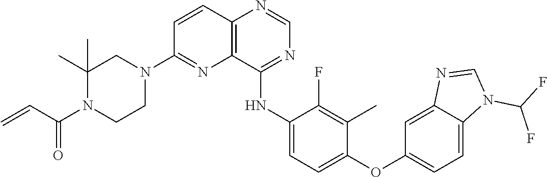 [SMILES: C=CC(=O)N1CCN(c2ccc3ncnc(Nc4ccc(Oc5ccc6c(c5)ncn6C(F)F)c(C)c4F)c3n2)CC1(C)C] | 1-(4-(4-((4-((1-(difluoromethyl)- 1H-benzo[d]imidazol-5-yl)oxy)-2- fluoro-3- methylphenyl)amino)pyrido[3,2- d]pyrimidin-6-y1)-2,2- dimethylpiperazin-1-yl)prop-2-en- 1-one | 603.4 (M + H) |
| 322 | 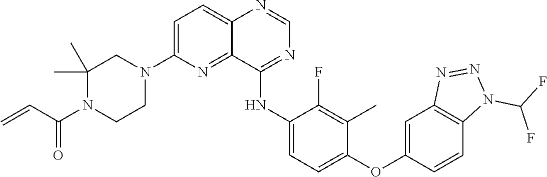 [SMILES: C=CC(=O)N1CCN(c2ccc3ncnc(Nc4ccc(Oc5ccc6c(c5)nnn6C(F)F)c(C)c4F)c3n2)CC1(C)C] | 1-(4-(4-((4-((1-(difluoromethyl)- 1H-benzo[d][1,2,3]triazol-5- yl)oxy)-2-fluoro-3- methylphenyl)amino)pyrido[3,2- d]pyrimidin-6-yl)-2,2- dimethylpiperazin-1-yl)prop-2-en- 1-one | 604.4 (M + H) |
| 323 | 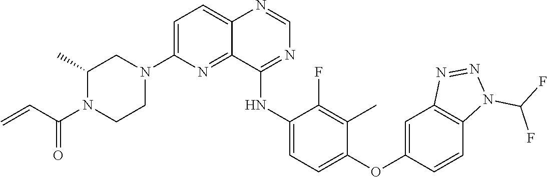 [SMILES: C=CC(=O)N1CCN(c2ccc3ncnc(Nc4ccc(Oc5ccc6c(c5)nnn6C(F)F)c(C)c4F)c3n2)C[C@H]1C] | (R)-1-(4-(4-((4-((1- (difluoromethyl)-1H- benzo[d][1,2,3]triazol-5-yl)oxy)-2- fluoro-3- methylphenyl)amino)pyrido[3,2- d]pyrimidin-6-yl)-2- methylpiperazin-1-yl)prop-2-en-1- one | 590.3 (M + H) |
| 324 | 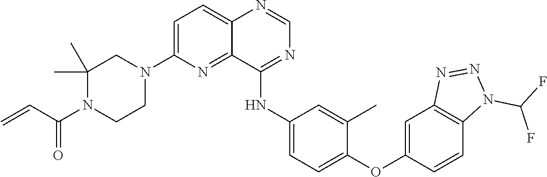 [SMILES: C=CC(=O)N1CCN(c2ccc3ncnc(Nc4ccc(Oc5ccc6c(c5)nnn6C(F)F)c(C)c4)c3n2)CC1(C)C] | 1-(4-(4-((4-((1-(difluoromethyl)- 1H-benzo[d][1,2,3]triazol-5- yl)oxy)-3- methylphenyl)amino)pyrido[3,2- d]pyrimidin-6-yl)-2,2- dimethylpiperazin-1-yl)prop-2-en- 1-one | 586.3 (M + H) |
| 325 | 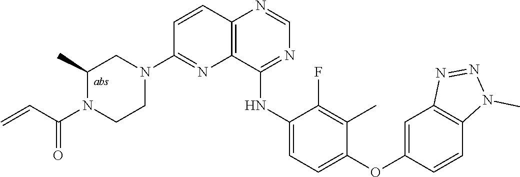 [SMILES: C=CC(=O)N1CCN(c2ccc3ncnc(Nc4ccc(Oc5ccc6c(c5)nnn6C)c(C)c4F)c3n2)C[C@@H]1C] | (S)-1-(4-(4-((2-fluoro-3-methyl-4- ((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 554.3 (M + H) |
| 326 | 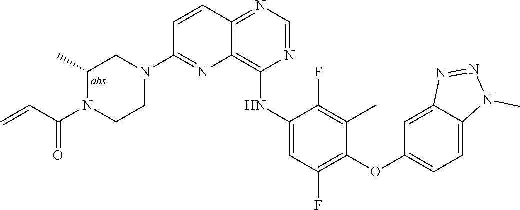 [SMILES: C=CC(=O)N1CCN(c2ccc3ncnc(Nc4cc(F)c(Oc5ccc6c(c5)nnn6C)c(C)c4F)c3n2)C[C@H]1C] | (R)-1-(4-(4-((2,5-difluoro-3- methyl-4-((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 572.4 (M + H) |
| 327 | 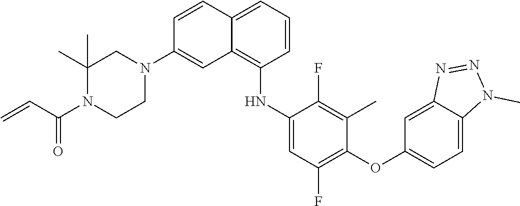 | 1-(4-(4-((2,5-difluoro-3-methyl-4- ((1-methyl-1H- benzo[d][1,2,3]triazol-5- yl)oxy)phenyl)amino)pyrido[3,2- d]pyrimidin-6-yl)-2,2- dimethylpiperazin-1-yl)prop-2-en- 1-one | 586.2 (M + H) |
| 328 | 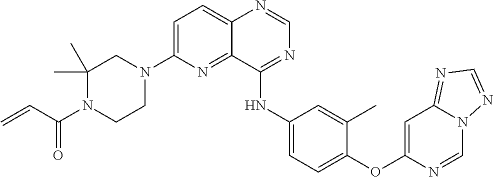 [SMILES: C=CC(=O)N1CCN(c2ccc3ncnc(Nc4ccc(Oc5cc6ncnn6cn5)c(C)c4)c3n2)CC1(C)C] | 1-(4-(4-((4-([1,2,4]triazolo[1,5- c]pyrimidin-7-yloxy)-3- methylphenyl)amino)pyrido[3,2- d]pyrimidin-6-yl)-2,2- dimethylpiperazin-1-yl)prop-2-en- 1-one | 537.3 (M + H) |
| 329 | 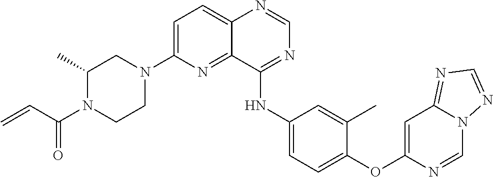 [SMILES: C=CC(=O)N1CCN(c2ccc3ncnc(Nc4ccc(Oc5cc6ncnn6cn5)c(C)c4)c3n2)C[C@H]1C] | (R)-1-(4-(4-((4-([1,2,4]triazolo[1,5- c]pyrimidin-7-yloxy)-3- methylphenyl)amino)pyrido[3,2- d]pyrimidin-6-yl)-2- methylpiperazin-1-yl)prop-2-en-1- one | 523.2 (M + H) |
| 330 | 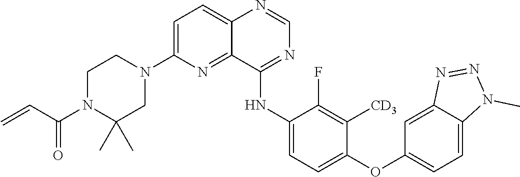 [SMILES: [2H]C([2H])([2H])c1c(Oc2ccc3c(c2)nnn3C)ccc(Nc2ncnc3ccc(N4CCN(C(=O)C=C)C(C)(C)C4)nc23)c1F] | 1-(4-(4-((2-fluoro-3-(methyl-d3)-4- ((1-methyl-1H- benzo[d][1,2,3]triazol-5- yl)oxy)phenyl)amino)pyrido[3,2- d]pyrimidin-6-yl)-2,2- dimethylpiperazin-1-yl)prop-2-en- 1-one | 571.3 (M + H) |
| 331 | 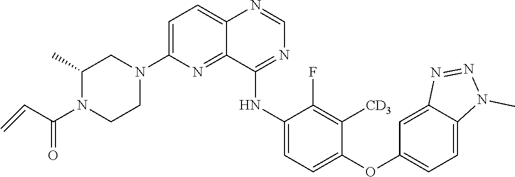 [SMILES: [2H]C([2H])([2H])c1c(Oc2ccc3c(c2)nnn3C)ccc(Nc2ncnc3ccc(N4CCN(C(=O)C=C)[C@H](C)C4)nc23)c1F] | (R)-1-(4-(4-((2-fluoro-3-(methyl- d3)-4-((1-methyl-1H- benzo[d][1,2,3]triazol-5- yl)oxy)phenyl)amino)pyrido[3,2- d]pyrimidin-6-yl)-2- methylpiperazin-1-yl)prop-2-en-1- one | 557.3 (M + H) |
| 332 | 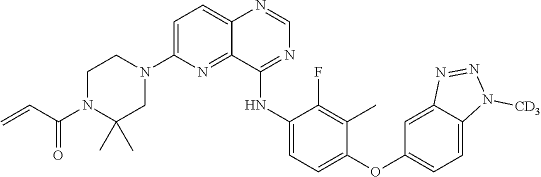 [SMILES: [2H]C([2H])([2H])n1nnc2cc(Oc3ccc(Nc4ncnc5ccc(N6CCN(C(=O)C=C)C(C)(C)C6)nc45)c(F)c3C)ccc21] | 1-(4-(4-((2-fluoro-3-methyl-4-((1- (methyl-d3)-1H- benzo[d][1,2,3]triazol-5- yl)oxy)phenyl)amino)pyrido[3,2- d]pyrimidin-6-yl)-2,2- dimethylpiperazin-1-yl)prop-2-en- 1-one | 571.3 (M + H) |
| 333 | 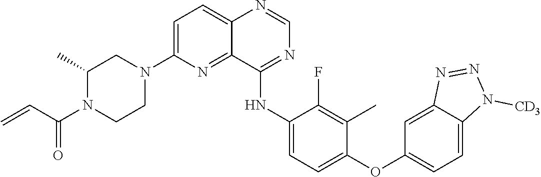 [SMILES: [2H]C([2H])([2H])n1nnc2cc(Oc3ccc(Nc4ncnc5ccc(N6CCN(C(=O)C=C)[C@H](C)C6)nc45)c(F)c3C)ccc21] | (R)-1-(4-(4-((2-fluoro-3-methyl-4- ((1-(methyl-d3)-1H- benzo[d][1,2,3]triazol-5- yl)oxy)phenyl)amino)pyrido[3,2- d]pyrimidin-6-yl)-2- methylpiperazin-1-yl)prop-2-en-1- one | 557.3 (M + H) |
| 334 | 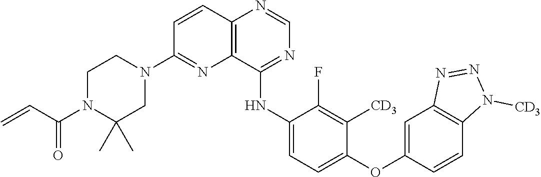 [SMILES: [2H]C([2H])([2H])c1c(Oc2ccc3c(c2)nnn3C([2H])([2H])[2H])ccc(Nc2ncnc3ccc(N4CCN(C(=O)C=C)C(C)(C)C4)nc23)c1F] | 1-(4-(4-((2-fluoro-3-(methyl-d3)-4- ((1-(methyl-d3)-1H- benzo[d][1,2,3]triazol-5- yl)oxy)phenyl)amino)pyrido[3,2- d]pyrimidin-6-yl)-2,2- dimethylpiperazin-1-yl)prop-2-en- 1-one | 574.4 (M + H) |

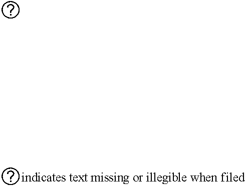

Compounds 335 to 340 in Table 20 below were prepared as a racemic mixture according to Example 68 Steps A to C. The enantiomers were separated using chiral supercritical fluid chromatography (SFC).

TABLE 20
335-340

| | | | MS |
|---|---|---|---|
| Cmp. | | | API |
| ID | Structure | Chemical Name | (m/z) |
| 335 | 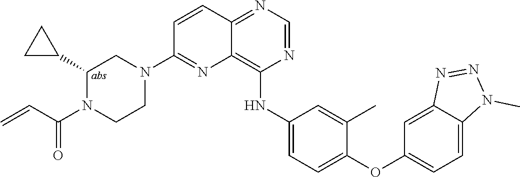 [SMILES: C=CC(=O)N1CCN(c2ccc3ncnc(Nc4ccc(Oc5ccc6c(c5)nnn6C)c(C)c4)c3n2)C[C@H]1C1CC1] | (R)-1-(2-cyclopropyl-4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 562.2 (M + H) |
| 336 | 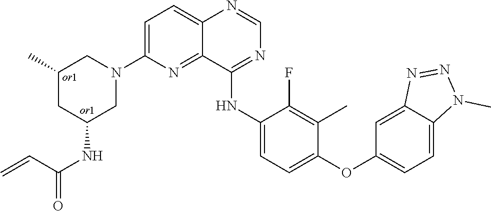 [SMILES: C=CC(=O)N[C@@H]1C[C@H](C)CN(c2ccc3ncnc(Nc4ccc(Oc5ccc6c(c5)nnn6C)c(C)c4F)c3n2)C1] | N-((3R,5S)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-5- methylpiperidin-3-yl)acrylamide | 568.3 (M + H) |
| 337 | 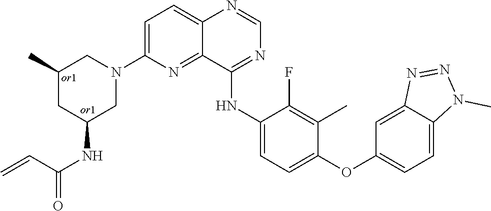 [SMILES: C=CC(=O)N[C@H]1C[C@@H](C)CN(c2ccc3ncnc(Nc4ccc(Oc5ccc6c(c5)nnn6C)c(C)c4F)c3n2)C1] | N-((3S,5R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-5- methylpiperidin-3-yl)acrylamide | 568.3 (M + H) |
| 338 | 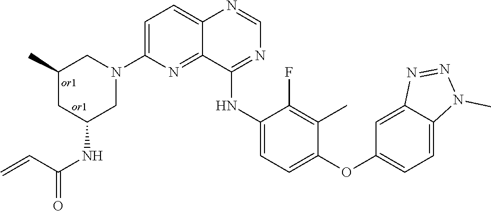 [SMILES: C=CC(=O)N[C@@H]1C[C@@H](C)CN(c2ccc3ncnc(Nc4ccc(Oc5ccc6c(c5)nnn6C)c(C)c4F)c3n2)C1] | N-((3R,5R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-5- methylpiperidin-3-yl)acrylamide | 568.2 (M + H) |
| 339 | 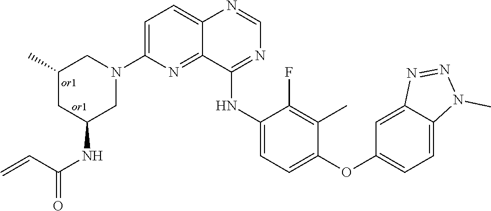 [SMILES: C=CC(=O)N[C@H]1C[C@H](C)CN(c2ccc3ncnc(Nc4ccc(Oc5ccc6c(c5)nnn6C)c(C)c4F)c3n2)C1] | N-((3S,5S)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-5- methylpiperidin-3-yl)acrylamide | 568.2 (M + H) |
| 340 | 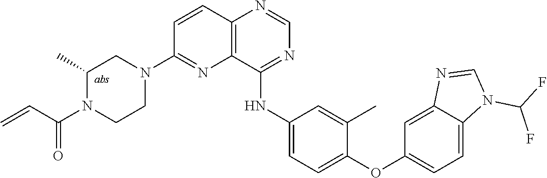 [SMILES: C=CC(=O)N1CCN(c2ccc3ncnc(Nc4ccc(Oc5ccc6c(c5)ncn6C(F)F)c(C)c4)c3n2)C[C@H]1C] | (R)-1-(4-(4-((4-((1-(difluoromethyl)- 1H-benzo[d]imidazol-5-yl)oxy)-3- methylphenyl)amino)pyrido[3,2- d]pyrimidin-6-yl)-2- methylpiperazin-1-yl)prop-2-en-1- one | 571.4 [M + H) |

### Example 69

### N-((3R,4R)-1-(4-((2-fluoro-3-methyl-4-((1-methyl-1H-benzo[d][1,2,3]triazol-5-yl)oxy)phenyl)amino)pyrido[3,2-d]pyrimidin-6-yl)-4-methylpiperidin-3-yl)acrylamide (compound 341)

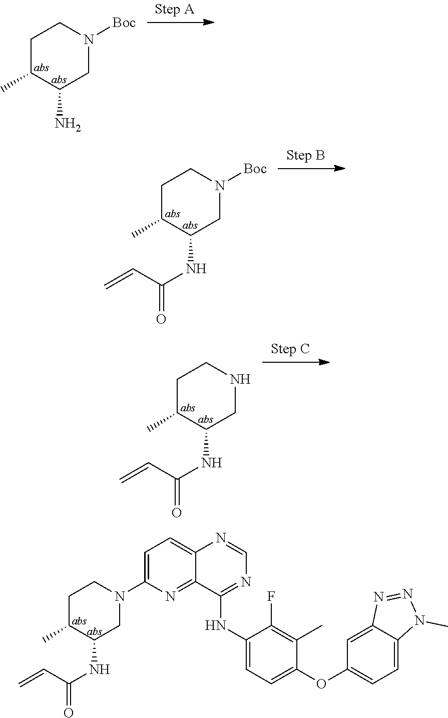

Step A. tert-butyl (3R,4R)-3-acrylamido-4-methylpiperidine-1-carboxylate. To solution of tert-butyl (3R,4R)-3-amino-4-methyl-piperidine-1-carboxylate (120 mg, 0.56 mmol) and TEA (0.24 mL, 1.72 mmol) in DCM (1 mL) was added prop-2-enoyl chloride (90 μL, 1.11 mmol). The reaction was stirred at 25° C. for 1 h. The mixture was concentrated under reduced pressure and the crude residue was purified by preparative TLC (50% EtOAc/PET) to afford tert-butyl (3R, 4R)-4-methyl-3-(prop-2-enoylamino) piperidine-1-carboxylate 3 (113 mg, 75% yield) as a light yellow solid. LCMS (MM-ES+APCI, Pos): m/z 169.3 (M+H-^{t}Bu).

Step B. N-((3R,4R)-4-methylpiperidin-3-yl)acrylamide trifluoroacetate. A solution of tert-butyl (3R, 4R)-4-methyl-3-(prop-2-enoylamino) piperidine-1-carboxylate (100 mg, 0.37 mmol) in DCM (2 mL) and TFA (1 mL) was stirred at 25° C. for 30 min. The reaction mixture was concentrated under reduced pressure to provide N-[(3R, 4R)-4-methyl-3-piperidyl]prop-2-enamide trifluoroacetate (150 mg, quant. yield) as a yellow oil. LCMS (MM-ES+APCI, Pos): m/z 168.9 (M+H).

Step C. N-((3R,4R)-1-(4-((2-fluoro-3-methyl-4-((1-methyl-1H-benzo[d][1,2,3]triazol-5-yl)oxy)phenyl)amino)pyrido[3,2-d]pyrimidin-6-yl)-4-methylpiperidin-3-yl)acrylamide. To solution of N-[(3R,4R)-4-methyl-3-piperidyl]prop-2-enamide trifluoroacetate (100 mg, 0.35 mmol) and 6-chloro-N-[2-fluoro-3-methyl-4-(1-methylbenzotriazol-5-yl)oxy-phenyl]pyrido[3,2-d]pyrimidin-4-amine (150 mg, 0.29 mmol) in DMSO (3 mL) were added KF (42 mg, 0.72 mmol) and DIPEA (0.24 mL, 1.37 mmol). The reaction was heated to 140° C. for 1 h. The mixture was filtered and the filtrate was purified by preparative HPLC (37% to 67% MeCN/0.08% aqueous NH₄HCO₃) and lyophilized to afford N-[(3R, 4R)-1-[4-[2-fluoro-3-methyl-4-(1-methylbenzotriazol-5-yl) oxy-anilino] pyrido[3, 2-d]pyrimidin-6-yl]-4-methyl-3-piperidyl] prop-2-enamide (81.5 mg, 40% yield) as a yellow solid. LCMS (MM-ES+APC), Pos): m/z 568.3 (M+H).

Compounds 341-350 in Table 21 below were synthesized according to Example 69 steps A to C. *Racemic mixture separated by SFC.

TABLE 21
Compounds 341-350

| | | | MS |
|---|---|---|---|
| Cmp. | | | API |
| ID | Structure | Chemical Name | (m/z) |
| 341 | 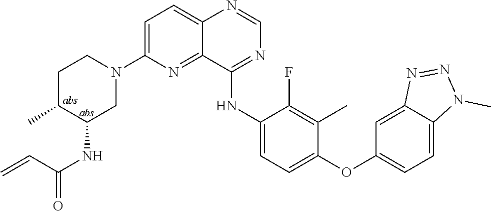 [SMILES: C=CC(=O)N[C@H]1CN(c2ccc3ncnc(Nc4ccc(Oc5ccc6c(c5)nnn6C)c(C)c4F)c3n2)CC[C@H]1C] | N-((3R,4R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-4- methylpiperidin-3-yl)acrylamide | 568.3 (M + H) |
| 342 | 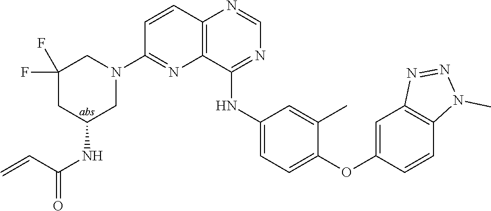 [SMILES: C=CC(=O)N[C@H]1CN(c2ccc3ncnc(Nc4ccc(Oc5ccc6c(c5)nnn6C)c(C)c4)c3n2)CC(F)(F)C1] | (R)-N-(5,5-difluoro-1-(4-((3-methyl- 4-((1-methyl-1H- benzo[d][1,2,3]triazol-5- yl)oxy)phenyl)amino)pyrido[3,2- d]pyrimidin-6-yl)piperidin-3- yl)acrylamide | 572.2 (M + H) |
| 343 | 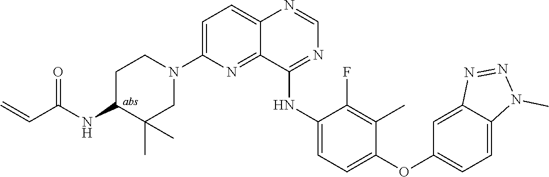 | (S)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)-3,3- dimethylpiperidin-4-yl)acrylamide | 582.2 (M + H) |
| 344 | 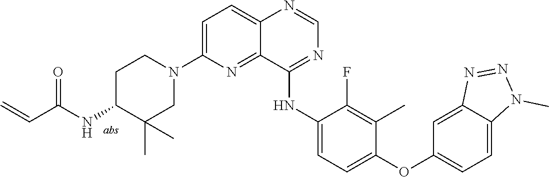 [SMILES: C=CC(=O)N[C@@H]1CCN(c2ccc3ncnc(Nc4ccc(Oc5ccc6c(c5)nnn6C)c(C)c4F)c3n2)CC1(C)C] | (R)-N-(1-(4-((2-fluoro-3-methyl-4- ((1-methyl-1H- benzo[d][1,2,3]triazol-5- yl)oxy)phenyl)amino)pyrido[3,2- d]pyrimidin-6-yl)-3,3- dimethylpiperidin-4-yl)acrylamide | 582.2 (M + H) |
| 345 | 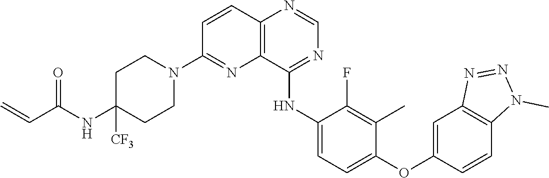 [SMILES: C=CC(=O)NC1(C(F)(F)F)CCN(c2ccc3ncnc(Nc4ccc(Oc5ccc6c(c5)nnn6C)c(C)c4F)c3n2)CC1] | N-(1-(4-((2-fluoro-3-methyl-4-((1- methyl-1H-benzo[d][1,2,3]triazol-5- yl)oxy)phenyl)amino)pyrido[3,2- d]pyrimidin-6-yl)-4- (trifluoromethyl)piperidin-4- yl)acrylamide | 622.1 (M + H) |
| 346 | 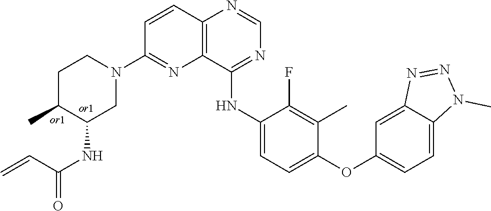 [SMILES: C=CC(=O)N[C@H]1CN(c2ccc3ncnc(Nc4ccc(Oc5ccc6c(c5)nnn6C)c(C)c4F)c3n2)CC[C@@H]1C] | N-((3R,4S)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-4- methylpiperidin-3-yl)acrylamide* | 568.3 (M + H) |
| 347 | 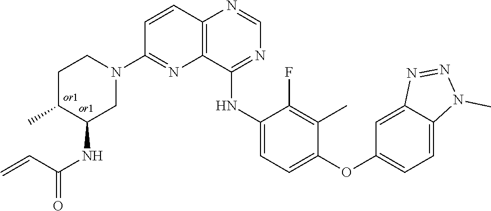 [SMILES: C=CC(=O)N[C@@H]1CN(c2ccc3ncnc(Nc4ccc(Oc5ccc6c(c5)nnn6C)c(C)c4F)c3n2)CC[C@H]1C] | N-((3S,4R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-4- methylpiperidin-3-yl)acrylamide* | 568.3 (M + H) |
| 348 | 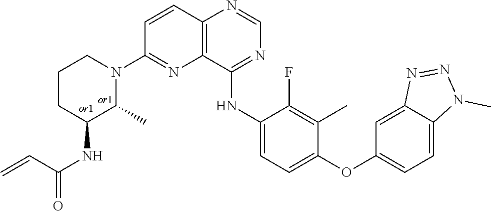 [SMILES: C=CC(=O)N[C@H]1CCCN(c2ccc3ncnc(Nc4ccc(Oc5ccc6c(c5)nnn6C)c(C)c4F)c3n2)[C@@H]1C] | N-((2R,3S)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-2- methylpiperidin-3-yl)acrylamide* | 568.2 (M + H) |
| 349 | 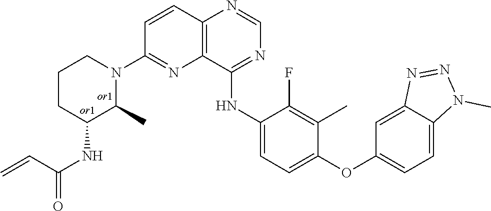 [SMILES: C=CC(=O)N[C@@H]1CCCN(c2ccc3ncnc(Nc4ccc(Oc5ccc6c(c5)nnn6C)c(C)c4F)c3n2)[C@H]1C] | N-((2S,3R)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-2- methylpiperidin-3-yl)acrylamide* | 568.2 (M + H) |
| 350 | 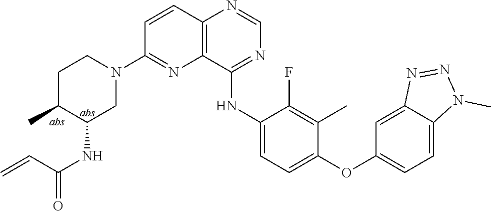 [SMILES: C=CC(=O)N[C@H]1CN(c2ccc3ncnc(Nc4ccc(Oc5ccc6c(c5)nnn6C)c(C)c4F)c3n2)CC[C@@H]1C] | N-((3R,4S)-1-(4-((2-fluoro-3- methyl-4-((1-methyl-1H- benzo[d][1,2,3]triazol-5- yl)oxy)phenyl)amino)pyrido[3,2- d]pyrimidin-6-yl)-4- methylpiperidin-3-yl)acrylamide | 568.2 (M + H) |

### Example 70

### 1-((2R,4S)-2-methyl-4-(methyl(4-((3-methyl-4-((1-methyl-H-benzo[d][1,2,3]triazol-5-yl)oxy)phenyl)amino)pyrido[3,2-d]pyrimidin-6-yl)amino)pyrrolidin-1-yl)prop-2-en-1-one (compound 351)

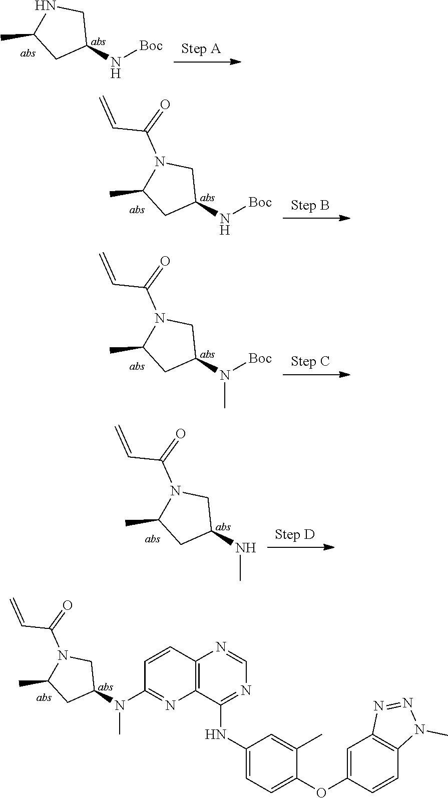

Step A. tert-butyl ((3S,5R)-1-acryloyl-5-methylpyrrolidin-3-yl)carbamate. Prepared according to Example 69 step A substituting tert-butyl ((3S,5R)-5-methylpyrrolidin-3-yl)carbamate for tert-butyl (3R,4R)-3-amino-4-methyl-piperidine-1-carboxylate to afford tert-butyl ((3S,5R)-1-acryloyl-5-methylpyrrolidin-3-yl)carbamate (120 mg, 95% yield) as a colorless oil.

Step B. tert-butyl ((3S,5R)-1-acryloyl-5-methylpyrrolidin-3-yl)(methyl)carbamate. To a solution of tert-butyl N-[(3S, 5R)-5-methyl-1-prop-2-enoyl-pyrrolidin-3-yl]carbamate (120 mg, 0.47 mmol) in THF (5 mL) at 0° C. were added NaH (28 mg, 0.71 mmol, 60% w/w) and iodomethane (59 μL, 0.94 mmol). The mixture was stirred at 25° C. for 0.5 h. The reaction was quenched with sat. aq. NH₄Cl (10 mL) and extracted with EtOAc (20 mL×3). The combined organic layers were washed with brine (10 mL), dried over anhydrous sodium sulfate, filtered, and concentrated under reduced pressure. The crude residue was purified by preparative TLC (100% EtOAc) to afford tert-butyl N-methyl-N-[(3S, 5R)-5-methyl-1-prop-2-enoyl-pyrrolidin-3-yl] carbamate (110 mg, 87% yield) as a yellow oil.

Steps C to D. 1-((2R,4S)-2-methyl-4-(methyl(4-((3-methyl-4-((1-methyl-1H-benzo[d][1,2,3]triazol-5-yl)oxy)phenyl)amino)pyrido[3,2-d]pyrimidin-6-yl)amino)pyrrolidin-1-yl)prop-2-en-1-one. Prepared according to Example 69 steps B to C, substituting tert-butyl ((3S,5R)-1-acryloyl-5-methylpyrrolidin-3-yl)(methyl)carbamate for tert-butyl (3R,4R)-3-acrylamido-4-methylpiperidine-1-carboxylate. LCMS (MM-ES+APCI, Pos): m/z 550.2 (M+H).

### Example 71

### 1-((2R,5S)-5-((4-((2-fluoro-3-methyl-4-((1-methyl-1H-benzo[d][1,2,3]triazol-5-yl)oxy)phenyl)amino)pyrido[3,2-d]pyrimidin-6-yl)(methyl)amino)-2-methylpiperidin-1-yl)prop-2-en-1-one (compound 352)

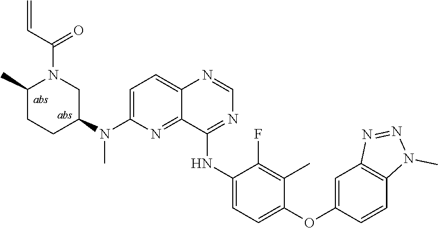

Steps A to D. 1-((2R,5S)-5-((4-((2-fluoro-3-methyl-4-((1-methyl-1H-benzo[d][1,2,3]triazol-5-yl)oxy)phenyl)amino)pyrido[3,2-d]pyrimidin-6-yl)(methyl)amino)-2-methylpiperidin-1-yl)prop-2-en-1-one. Prepared according to Example 70 steps A to D, substituting tert-butyl ((3S,6R)-6-methylpiperidin-3-yl)carbamate for tert-butyl ((3S,5R)-5-methylpyrrolidin-3-yl)carbamate and 6-fluoro-N-(2-fluoro-3-methyl-4-((1-methyl-1H-benzo[d][1,2,3]triazol-5-yl)oxy)phenyl)pyrido[3,2-d]pyrimidin-4-amine for 6-chloro-N-[2-fluoro-3-methyl-4-(1-methylbenzotriazol-5-yl)oxy-phenyl]pyrido[3,2-d]pyrimidin-4-amine to afford 1-((2R,5S)-5-((4-((2-fluoro-3-methyl-4-((1-methyl-1H-benzo[d][1,2,3]triazol-5-yl)oxy)phenyl)amino)pyrido[3,2-d]pyrimidin-6-yl)(methyl)amino)-2-methylpiperidin-1-yl)prop-2-en-1-one (38.8 mg, 14% yield) as a yellow solid. LCMS (MM-ES+APCI, Pos): m/z 582.4 (M+H).

### Example 72

### (S)-2-(1-acryloyl-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (compound 83)

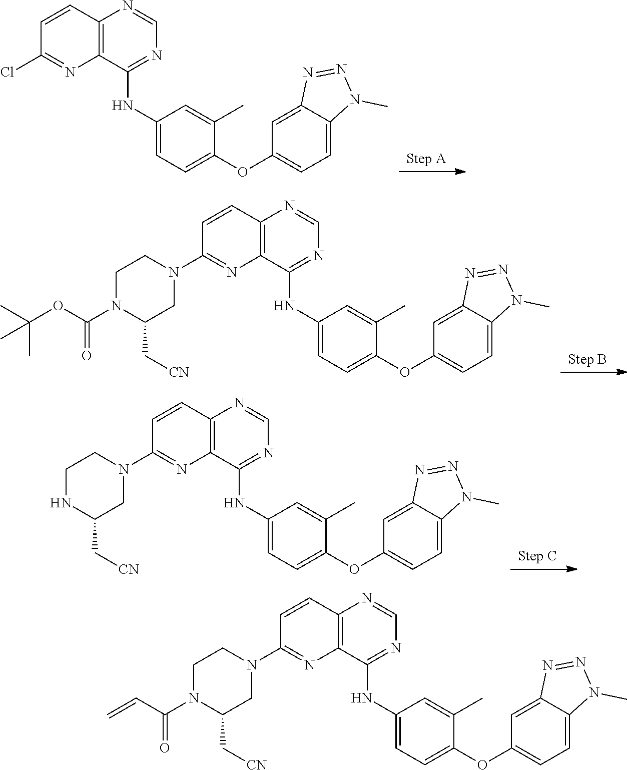

Step A. tert-butyl (S)-2-(cyanomethyl)-4-(4-((3-methyl-4-((1-methyl-1H-benzol[d][1,2,3]triazol-5-yl)oxy)phenyl)amino)pyrido[3,2-d]pyrimidin-6-yl)piperazine-1-carboxylate. A solution of 6-chloro-N-(3-methyl-4-((1-methyl-H-benzo[d][1,2,3]triazol-5-yl)oxy)phenyl)pyrido[3,2-d]pyrimidin-4-amine (20 mg, 48 μmol), tert-butyl (S)-2-(cyanomethyl)piperazine-1-carboxylate (13 mg, 57 μmol), Cs₂CO₃ (31 mg, 96 μmol), and dichloro[1,3-bis(2,6-di-3-pentylphenyl)imidazol-2-ylidene](3-chloropyridyl)palladium(II) (9 mg, 10 μmol) in 1,4-dioxane (0.24 mL) was heated to 90° C. for 16 h. The reaction mixture was concentrated in vacuo and the residue purified via silica gel chromatography (0 to 10% MeOH in DCM) to obtain tert-butyl (S)-2-(cyanomethyl)-4-(4-((3-methyl-4-((1-methyl-1H-benzo[d][1,2,3]triazol-5-yl)oxy)phenyl)amino)pyrido[3,2-d]pyrimidin-6-yl)piperazine-1-carboxylate (9.0 mg, 31%) as a yellow solid. LCMS (MM-ES+APCI, Pos): m/z 607.3 (M+H).

Step B. (S)-2-(4-(4-((3-methyl-4-((1-methyl-H-benzo[d][1,2,3]triazol-5-yl)oxy)phenyl)amino)pyrido[3,2-d]pyrimidin-6-yl)piperazin-2-yl)acetonitrile. Prepared according to Example 68 Step B, substituting tert-butyl (S)-2-(cyanomethyl)-4-(4-((3-methyl-4-((1-methyl-1H-benzo[d][1,2,3]triazol-5-yl)oxy)phenyl)amino)pyrido[3,2-d]pyrimidin-6-yl)piperazine-1-carboxylate for tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate to provide (S)-2-(4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (11.3 mg, 64%) as a yellow solid. LCMS (MM-ES+APCI, Pos): m/z 507.3 (M+H).

Step C. (S)-2-(1-acryloyl-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile. Prepared according to Example 68 Step C, substituting (S)-2-(4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile for 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine to give (S)-2-(1-acryloyl-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (3.9 mg, 31%) as a yellow solid. LCMS (MM-ES+APCI, Pos): m/z 561.3 (M+H).

Compounds 83, 353-357 in Table 22 below were synthesized according to Example 72 steps A to C.

TABLE 22
Compounds 83, 353-357

| | | | MS |
|---|---|---|---|
| Cmp. | | | API |
| ID | Structure | Chemical Name | (m/z) |
| 83 | 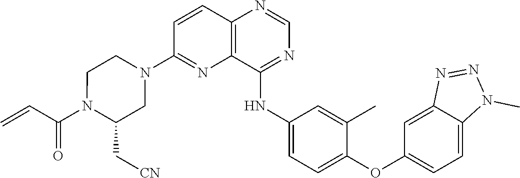 [SMILES: C=CC(=O)N1CCN(c2ccc3ncnc(Nc4ccc(Oc5ccc6c(c5)nnn6C)c(C)c4)c3n2)C[C@@H]1CC#N] | (S)-2-(1-acryloyl-4-(4-((3-methyl-4- ((1-methyl-1H- benzo[d][1,2,3]triazol-5- yl)oxy)phenyl)amino)pyrido[3,2- d]pyrimidin-6-yl)piperazin-2- yl)acetonitrile | 561.3 (M + H) |
| 353 | 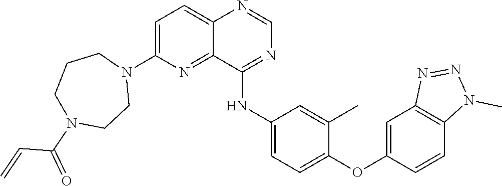 [SMILES: C=CC(=O)N1CCCN(c2ccc3ncnc(Nc4ccc(Oc5ccc6c(c5)nnn6C)c(C)c4)c3n2)CC1] | 1-(4-(4-((3-methyl-4-((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 536.3 (M + H) |
| 354 | 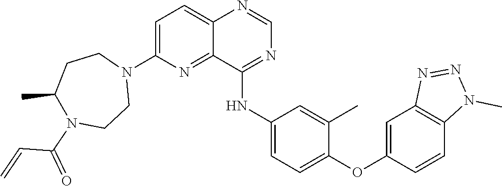 [SMILES: C=CC(=O)N1CCN(c2ccc3ncnc(Nc4ccc(Oc5ccc6c(c5)nnn6C)c(C)c4)c3n2)CC[C@@H]1C] | (S)-1-(7-methyl-4-(4-((3-methyl-4- ((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 550.3 (M + H) |
| 355 | 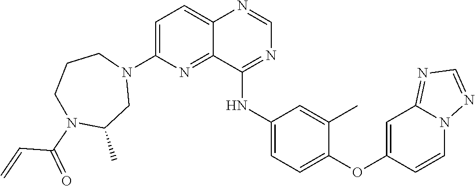 [SMILES: C=CC(=O)N1CCCN(c2ccc3ncnc(Nc4ccc(Oc5ccn6ncnc6c5)c(C)c4)c3n2)C[C@@H]1C] | (S)-1-(4-(4-((4-([1,2,4]triazolo[1,5- a]pyridin-7-yloxy)-3- methylphenyl)amino)pyrido[3,2- d]pyrimidin-6-yl)-2-methyl-1,4- diazepan-1-yl)prop-2-en-1-one | 536.3 |
| 356 | 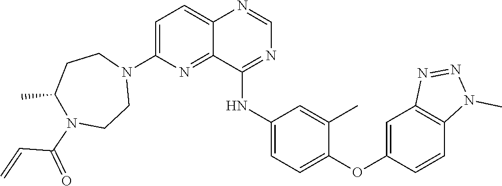 [SMILES: C=CC(=O)N1CCN(c2ccc3ncnc(Nc4ccc(Oc5ccc6c(c5)nnn6C)c(C)c4)c3n2)CC[C@H]1C] | (R)-1-(7-methyl-4-(4-((3-methyl-4- ((1-methyl-1H- benzo[d][1,2,3]triazol-5- yl)oxy)phenyl)amino)pyrido[3,2- d]pyrimidin-6-yl)-1,4-diazepan-1- yl)prop-2-en-1-one | 550.3 (M + H) |
| 357 | 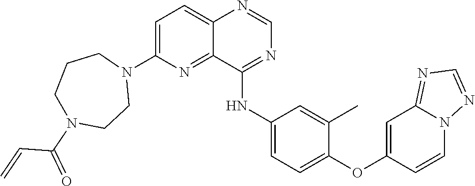 | 1-(4-(4-((4-([1,2,4]triazolo[1,5- a]pyridin-7-yloxy)-3- methylphenyl)amino)pyrido[3,2- d]pyrimidin-6-yl)-1,4-diazepan-1- yl)prop-2-en-1-one | 568.3 (M + H) |

### Example 73

### (R)-2-(1-acryloyl-4-(4-((3-methyl-4-((1-methyl-H-benzo[d][1,2,3]triazol-5-yl)oxy)phenyl)amino)pyrido[3,2-d]pyrimidin-6-yl)piperazin-2-yl)acetonitrile (compound 84)

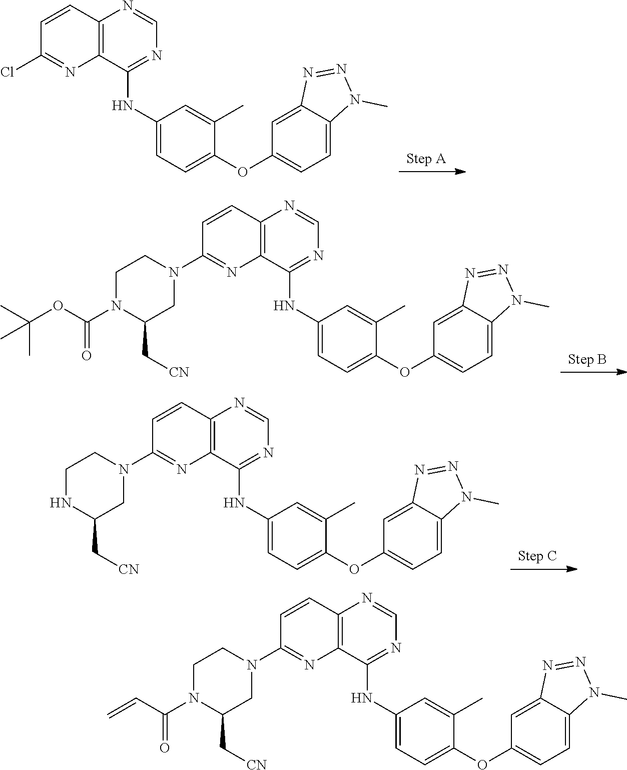

(R)-2-(1-acryloyl-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile. Prepared according to Example 72 Steps A to C, substituting tert-butyl (R)-2-(cyanomethyl)piperazine-1-carboxylate for tert-butyl (S)-2-(cyanomethyl)piperazine-1-carboxylate to afford (R)-2-(1-acryloyl-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (11.20 mg, 19%) as a yellow solid. LCMS (MM-ES+APCI, Pos): m/z 561.3 (M+H).

### Example 74

### (S)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (compound 85)

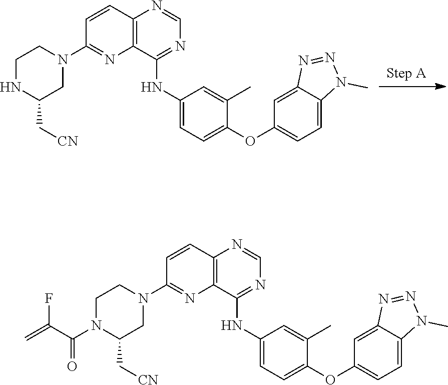

Step A. (S)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile. To a solution of (S)-2-(4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (60.0 mg, 0.12 mmol), 2-fluoroacrylic acid (24 mg, 0.26 mmol), and HATU (99 mg, 0.26 mmol) in DMF (1.2 mL) was added DIPEA (0.10 mL, 0.59 mmol). The reaction was stirred for 1.5 h, diluted with 25% IPA/CHCl₃, and washed with H₂O. The organic phase was dried with Na₂SO₄, filtered, and concentrated in vacuo. The resulting residue was purified via preparative HPLC (5% to 100% MeCN/0.1% aqueous TFA). The product was neutralized with sat. aq. NaHCO₃ and extracted with 25% IPA/CHCl₃ to provide (S)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (29.43 mg, 43%) as a yellow solid. LCMS (MM-ES+APCI, Pos): m/z 579.3 (M+H).

### Example 75

### (R)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (compound 86)

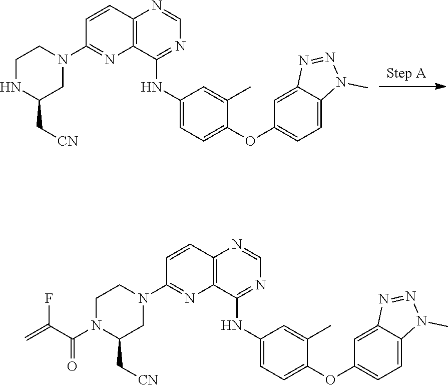

(R)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile. Prepared according to Example 74 Step A substituting (R)-2-(4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile for (S)-2-(4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile to yield (R)-2-(1-(2-fluoroacryloyl)-4-(4-((3-methyl-4-((1-methyl-1H-benzo[d][1,2,3]triazol-5-yl)oxy)phenyl)amino)pyrido[3,2-d]pyrimidin-6-yl)piperazin-2-yl)acetonitrile (37.7 mg, 47%) as a yellow solid. LCMS (MM-ES+APCI, Pos): m/z 579.3 (M+H).

### Example 76

### 2-fluoro-1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-1-one (compound 87)

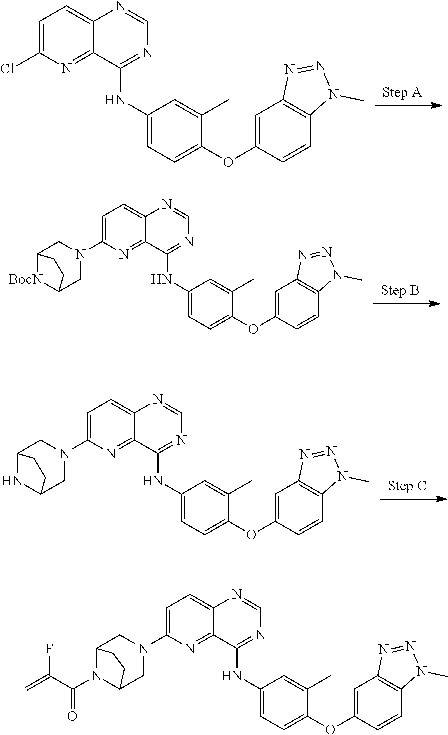

Step A. tert-butyl 3-(4-((3-methyl-4-((1-methyl-H-benzo[d][1,2,3]triazol-5-yl)oxy)phenyl)amino)pyrido[3,2-d]pyrimidin-6-yl)-3,8-diazabicyclo[3.2.1]octane-8-carboxylate. To a slurry of 6-chloro-N-(3-methyl-4-((1-methyl-1H-benzo[d][1,2,3]triazol-5-yl)oxy)phenyl)pyrido[3,2-d]pyrimidin-4-amine (40 mg, 0.01 mmol) and tert-butyl 3,8-diazabicyclo[3.2.1]octane-8-carboxylate (31 mg, 0.14 mmol) in 1,4-dioxane (1.0 mL) was added DIPEA (42 μL, 0.24 mmol). The reaction was heated to 110° C. and stirred for 2 h. DMSO (1 mL) was added and the reaction was heated to 140° C. for 14 h. The mixture was diluted with water (10 mL), extracted with 20% MeOH/DCM (10 mL×6), dried over anhydrous MgSO₄, filtered, and concentrated in vacuo. The crude product was purified by silica gel chromatography (0% to 20% MeOH/DCM) to afford tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate (38 mg, 67% yield). LCMS (MM-ES+APCI, Pos): m/z 594.4 (M+H).

Step B. 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine. Prepared according to Example 68 Step B, substituting tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate for tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate to give 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine (13.9 mg, 45% yield). LCMS (MM-ES+APCI, Pos): m/z 494.3 (M+H).

Step C. 2-fluoro-1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-1-one. Prepared according to Example 56 Step C, substituting 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine for 6-(2,5-diazabicyclo[2.2.2]octan-2-yl)-N-(3-methyl-4-((1-methyl-1H-benzo[d][1,2,3]triazol-5-yl)oxy)phenyl)pyrimido[5,4-d]pyrimidin-4-amine to yield 2-fluoro-1-(3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octan-8-yl)prop-2-en-a-one (8.7 mg, 52% yield). LCMS (MM-ES+APC, Pos): m/z 566.3 (M+H).

Compounds 87, 88, 358-369 in Table 23 below were synthesized according to Example 76 steps A to C.

TABLE 23
Compounds 87, 88, 358-369

| | | | MS |
|---|---|---|---|
| Cmp. | | | API |
| ID | Structure | Chemical Name | (m/z) |
| 87 | 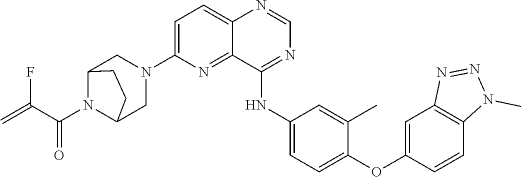 [SMILES: C=C(F)C(=O)N1C2CCC1CN(c1ccc3ncnc(Nc4ccc(Oc5ccc6c(c5)nnn6C)c(C)c4)c3n1)C2] | 2-fluoro-1-(3-(4-((3-methyl-4-((1- methyl-1H-benzo[d][1,2,3]triazol-5- yl)oxy)phenyl)amino)pyrido[3,2- d]pyrimidin-6-yl)-3,8- diazabicyclo[3.2.1]octan-8-yl)prop- 2-en-1-one | 566.3 (M + H) |
| 88 | 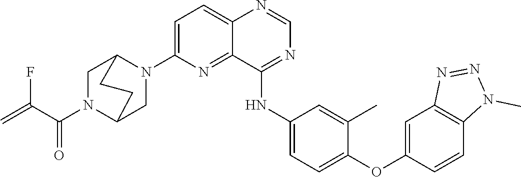 [SMILES: C=C(F)C(=O)N1CC2CCC1CN2c1ccc2ncnc(Nc3ccc(Oc4ccc5c(c4)nnn5C)c(C)c3)c2n1] | 2-fluoro-1-(5-(4-((3-methyl-4-((1- methyl-1H-benzo[d][1,2,3]triazol-5- yl)oxy)phenyl)amino)pyrido[3,2- d]pyrimidin-6-yl)-2,5- diazabicyclo[2.2.2]octan-2-yl)prop- 2-en-1-one | 566.3 (M + H) |
| 358 | 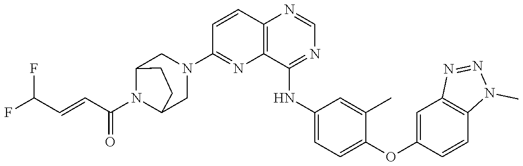 [SMILES: Cc1cc(Nc2ncnc3ccc(N4CC5CCC(C4)N5C(=O)/C=C/C(F)F)nc23)ccc1Oc1ccc2c(c1)nnn2C] | (E)-4,4-difluoro-1-(3-(4-((3-methyl- 4-((1-methyl-1H- benzo[d][1,2,3]triazol-5- yl)oxy)phenyl)amino)pyrido[3,2- d]pyrimidin-6-yl)-3,8- diazabicyclo[3.2.1]octan-8-yl)but-2- en-1-one | 598.3 (M + H) |
| 359 | 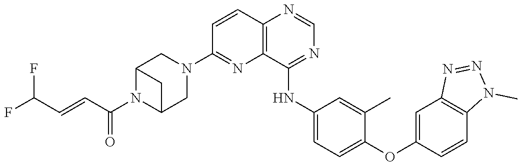 [SMILES: Cc1cc(Nc2ncnc3ccc(N4CC5CC(C4)N5C(=O)/C=C/C(F)F)nc23)ccc1Oc1ccc2c(c1)nnn2C] | (E)-4,4-difluoro-1-(3-(4-((3-methyl- 4-((1-methyl-1H- benzo[d][1,2,3]triazol-5- yl)oxy)phenyl)amino)pyrido[3,2- d]pyrimidin-6-yl)-3,6- diazabicyclo[3.1.1]heptan-6-yl)but- 2-en-1-one | 584.3 (M + H) |
| 360 | 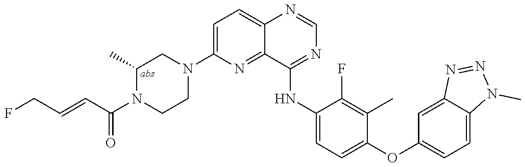 [SMILES: Cc1c(Oc2ccc3c(c2)nnn3C)ccc(Nc2ncnc3ccc(N4CCN(C(=O)/C=C/CF)[C@H](C)C4)nc23)c1F] | (R,E)-4-fluoro-1-(4-(4-((2-fluoro-3- methyl-4-((1-methyl-1H- benzo[d][1,2,3]triazol-5- yl)oxy)phenyl)amino)pyrido[3,2- d]pyrimidin-6-yl)-2- methylpiperazin-1-yl)but-2-en-1-one | 586.4 (M + H) |
| 361 | 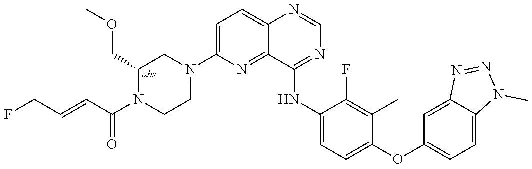 [SMILES: COC[C@@H]1CN(c2ccc3ncnc(Nc4ccc(Oc5ccc6c(c5)nnn6C)c(C)c4F)c3n2)CCN1C(=O)/C=C/CF] | (S,E)-4-fluoro-1-(4-(4-((2-fluoro-3- methyl-4-((1-methyl-1H- benzo[d][1,2,3]triazol-5- yl)oxy)phenyl)amino)pyrido[3,2- d]pyrimidin-6-yl)-2- (methoxymethyl)piperazin-1-yl)but- 2-en-1-one | 616.5 (M + H) |
| 362 | 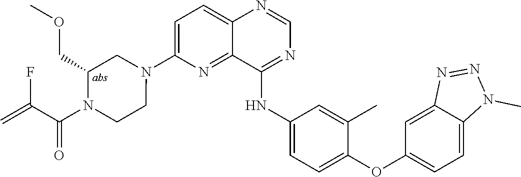 [SMILES: C=C(F)C(=O)N1CCN(c2ccc3ncnc(Nc4ccc(Oc5ccc6c(c5)nnn6C)c(C)c4)c3n2)C[C@H]1COC] | (S)-2-fluoro-1-(2-(methoxymethyl)- 4-(4-((3-methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 584.3 (M + H) |
| 363 | 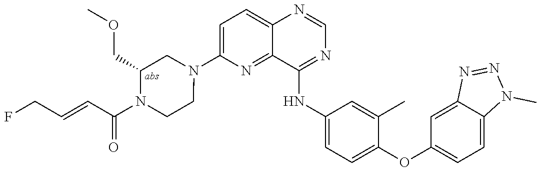 [SMILES: COC[C@@H]1CN(c2ccc3ncnc(Nc4ccc(Oc5ccc6c(c5)nnn6C)c(C)c4)c3n2)CCN1C(=O)/C=C/CF] | (S,E)-4-fluoro-1-(2- (methoxymethyl)-4-(4-((3-methyl-4- ((1-methyl-1H- benzo[d][1,2,3]triazol-5- yl)oxy)phenyl)amino)pyrido[3,2- d]pyrimidin-6-yl)piperazin-1-yl)but- 2-en-1-one | 598.2 (M + H) |
| 364 | 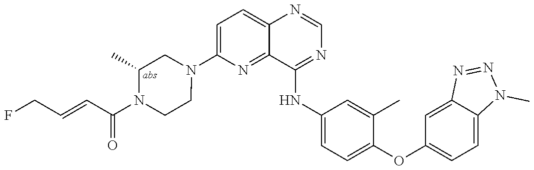 [SMILES: Cc1cc(Nc2ncnc3ccc(N4CCN(C(=O)/C=C/CF)[C@H](C)C4)nc23)ccc1Oc1ccc2c(c1)nnn2C] | (R,E)-4-fluoro-1-(2-methyl-4-(4-((3- methyl-4-((1-methyl-1H- benzo[d][1,2,3]triazol-5- yl)oxy)phenyl)amino)pyrido[3,2- d]pyrimidin-6-yl)piperazin-1-yl)but- 2-en-1-one | 568.2 (M + H) |
| 365 | 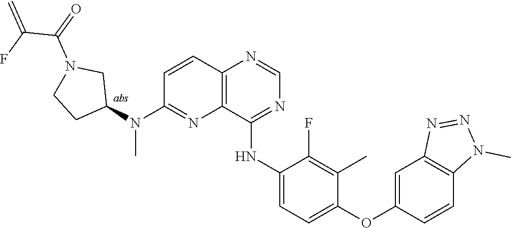 [SMILES: C=C(F)C(=O)N1CC[C@H](N(C)c2ccc3ncnc(Nc4ccc(Oc5ccc6c(c5)nnn6C)c(C)c4F)c3n2)C1] | (S)-2-fluoro-1-(3-((4-((2-fluoro-3- methyl-4-((1-methyl-1H- benzo[d][1,2,3]triazol-5- yl)oxy)phenyl)amino)pyrido[3,2- d]pyrimidin-6- yl)(methyl)amino)pyrrolidin-1- yl)prop-2-en-1-one | 572.2 (M + H) |
| 366 | 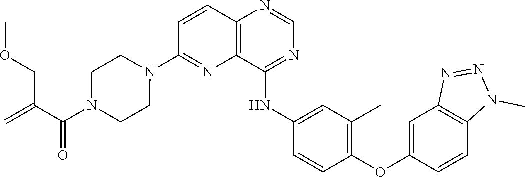 [SMILES: C=C(COC)C(=O)N1CCN(c2ccc3ncnc(Nc4ccc(Oc5ccc6c(c5)nnn6C)c(C)c4)c3n2)CC1] | 2-(methoxymethyl)-1-(4-(4-((3- methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 565.3 (M + H) |
| 367 | 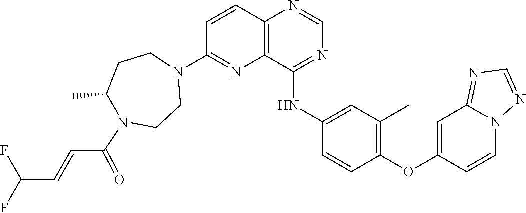 [SMILES: Cc1cc(Nc2ncnc3ccc(N4CC[C@@H](C)N(C(=O)/C=C/C(F)F)CC4)nc23)ccc1Oc1ccn2ncnc2c1] | (R,E)-1-(4-(4-((4- ([1,2,4]triazolo[1,5-a]pyridin-7- yloxy)-3- methylphenyl)amino)pyrido[3,2- d]pyrimidin-6-yl)-7-methyl-1,4- diazepan-1-y1)-4,4-difluorobut-2-en- 1-one | 586.3 (M + H) |
| 368 | 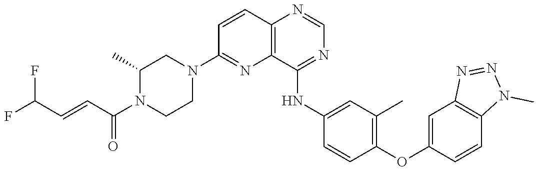 [SMILES: Cc1cc(Nc2ncnc3ccc(N4CCN(C(=O)/C=C/C(F)F)[C@H](C)C4)nc23)ccc1Oc1ccc2c(c1)nnn2C] | (R,E)-4,4-difluoro-1-(2-methyl-4-(4- ((3-methyl-4-((1-methyl-1H- benzo[d][1,2,3]triazol-5- yl)oxy)phenyl)amino)pyrido[3,2- d]pyrimidin-6-yl)piperazin-1-yl)but- 2-en-1-one | 586.3 (M + H) |
| 369 | 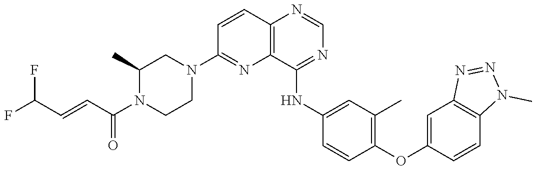 [SMILES: Cc1cc(Nc2ncnc3ccc(N4CCN(C(=O)/C=C/C(F)F)[C@@H](C)C4)nc23)ccc1Oc1ccc2c(c1)nnn2C] | (S,E)-4,4-difluoro-1-(2-methyl-4-(4- ((3-methyl-4-((1-methyl-1H- benzo[d][1,2,3]triazol-5- yl)oxy)phenyl)amino)pyrido[3,2- d]pyrimidin-6-yl)piperazin-1-yl)but- 2-en-1-one | 586.3 (M + H) |

### Example 77

### 1-(2,2-dimethyl-4-(4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (compound 89)

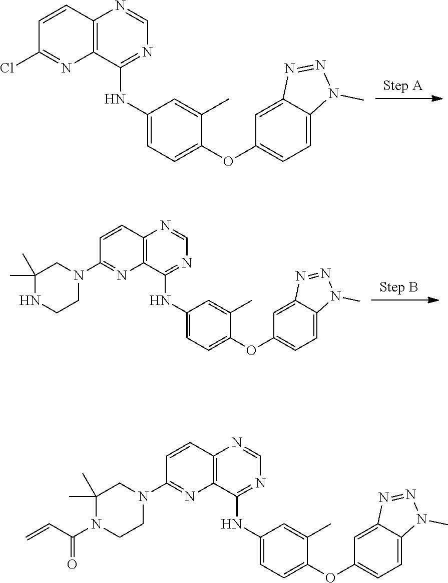

Step A. 6-(3,3-dimethylpiperazin-1-yl)-N-(3-methyl-4-((1-methyl-1H-benzo[d][1,2,3]triazol-5-yl)oxy)phenyl)pyrido[3,2-d]pyrimidin-4-amine. A mixture of 6-chloro-N-(3-methyl-4-((1-methyl-1H-benzo[d][1,2,3]triazol-5-yl)oxy)phenyl)pyrido[3,2-d]pyrimidin-4-amine (100 mg, 0.24 mmol), tert-butyl 2,2-dimethylpiperazine-1-carboxylate (103 mg, 0.48 mmol), and Cs₂CO₃ (234 mg, 0.72 mmol) in DMF (0.8 mL) was heated to 110° C. for 18 h. The reaction was diluted with EtOAc and washed with H₂O and brine. The organic phase was dried over Na₂SO₄, filtered, and concentrated in vacuo. The crude residue was purified via silica gel chromatography (0% to 20% MeOH/DCM) to obtain 6-(3,3-dimethylpiperazin-1-yl)-N-(3-methyl-4-((1-methyl-1H-benzo[d][1,2,3]triazol-5-yl)oxy)phenyl)pyrido[3,2-d]pyrimidin-4-amine (8.6 mg, 7%). LCMS (MM-ES+APCI, Pos): m/z 496.3 (M+H).

Step B. 1-(2,2-dimethyl-4-(4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one. To a solution of 6-(3,3-dimethylpiperazin-1-yl)-N-(3-methyl-4-((1-methyl-1H-benzo[d][1,2,3]triazol-5-yl)oxy)phenyl)pyrido[3,2-d]pyrimidin-4-amine (8.6 mg, 17 μmol) and TEA (5 μL, 35 μmol) in DCM (0.17 mL) at 0° C. was added acryloyl chloride (0.2M in DCM, 0.10 mL, 20 μmol). The reaction was stirred for 10 min, quenched with sat. aq. NaHCO₃ and extracted with DCM. The combined organic phases were dried over Na₂SO₄, filtered, and concentrated in vacuo. The residue was purified via preparative HPLC (5% to 100% MeCN/0.1% aqueous TFA). The product was neutralized with sat. aq. NaHCO₃ and extracted with 20% MeOH in DCM to afford 1-(2,2-dimethyl-4-(4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (5.7 mg, 59%) as a yellow solid. LCMS (MM-ES+APCI, Pos): m/z 550.3 (M+H).

### Example 78

### (S)-1-(3-methyl-4-(4-((3-methyl-4-((1-methyl-H-benzo[d][1,2,3]triazol-5-yl)oxy)phenyl)amino)pyrido[3,2-d]pyrimidin-6-yl)piperazin-1-yl)prop-2-en-1-one 2,2,2-trifluoroacetate (compound 90)

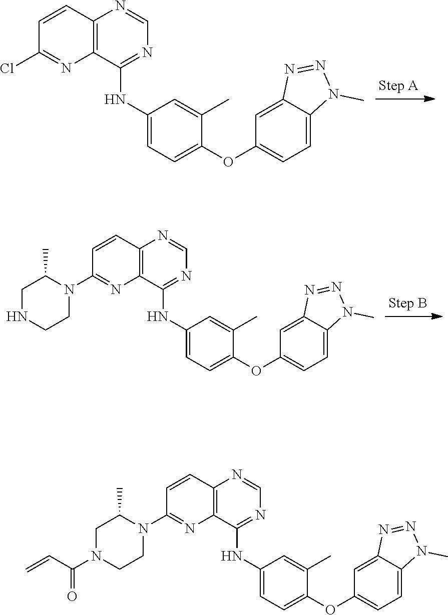

Step A. (S)—N-(3-methyl-4-((1-methyl-1H-benzo[d][1,2,3]triazol-5-yl)oxy)phenyl)-6-(2-methylpiperazin-1-yl)pyrido[3,2-d]pyrimidin-4-amine. 6-chloro-N-(3-methyl-4-((1-methyl-1H-benzo[d][1,2,3]triazol-5-yl)oxy)phenyl)pyrido[3,2-d]pyrimidin-4-amine (50 mg, 0.12 mmol), tert-butyl (S)-3-methylpiperazine-1-carboxylate (96 mg, 0.48 mmol) and Cs₂CO₃ (39 mg, 0.12 mmol) were dissolved in DMSO (1.1 mL). The reaction was stirred for 2 h at 115° C. and for 16 h at 150° C. The mixture was concentrated in vacuo and purified by silica gel chromatography (0% to 20% MeOH/DCM) to afford tert-butyl (S)-3-methyl-4-(4-((3-methyl-4-((1-methyl-1H-benzo[d][1,2,3]triazol-5-yl)oxy)phenyl)amino)pyrido[3,2-d]pyrimidin-6-yl)piperazine-1-carboxylate (60 mg, 77% yield). LCMS (MM-ES+APCI, Pos): m/z 482.3 (M+H).

Step B. (S)-1-(3-methyl-4-(4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one 2,2,2-trifluoroacetate. Prepared according to Example 77 Step B, substituting (S)—N-(3-methyl-4-((1-methyl-1H-benzo[d][1,2,3]triazol-5-yl)oxy)phenyl)-6-(2-methylpiperazin-1-yl)pyrido[3,2-d]pyrimidin-4-amine (25 mg, 52 μmol) for 6-(3,3-dimethylpiperazin-1-yl)-N-(3-methyl-4-((1-methyl-1H-benzo[d][1,2,3]triazol-5-yl)oxy)phenyl)pyrido[3,2-d]pyrimidin-4-amine to afford (S)-1-(3-methyl-4-(4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one 2,2,2-trifluoroacetate (8.5 mg, 24% yield). LCMS (MM-ES+APCI, Pos): m/z 536.3 (M+H).

### Example 79

### 1-(4-(4-((2-fluoro-4-((7-fluoro-1-methyl-1H-benzo[d][1,2,3]triazol-5-yl)oxy)-3-methylphenyl)amino)pyrido[3,2-d]pyrimidin-6-yl)piperazin-1-yl)prop-2-en-1-one (compound 91)

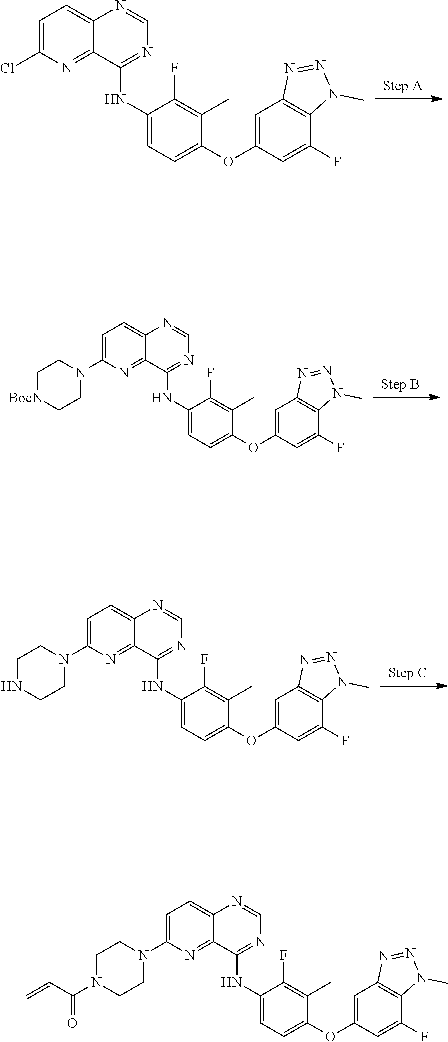

Step A. tert-butyl 4-(4-((2-fluoro-4-((7-fluoro-1-methyl-H-benzo[d][1,2,3]triazol-5-yl)oxy)-3-methylphenyl)amino)pyrido[3,2-d]pyrimidin-6-yl)piperazine-1-carboxylate. To a solution of 6-chloro-N-[2-fluoro-4-(7-fluoro-1-methyl-benzotriazol-5-yl)oxy-3-methyl-phenyl]pyrido[3,2-d]pyrimidin-4-amine (95 mg, 0.21 mmol) and tert-butyl piperazine-1-carboxylate (39 mg, 0.21 mmol) in DMF (5 mL) was added DIPEA (0.11 mL, 0.63 mmol). The mixture was stirred at 120° C. for 6 h, diluted with water (10 mL), and extracted with EtOAc (15 mL×3). The combined organic layers were washed with brine (20 mL), dried over anhydrous Na₂SO₄, filtered and concentrated in vacuo to provide tert-butyl 4-[4-[2-fluoro-4-(7-fluoro-1-methyl-benzotriazol-5-yl)oxy-3-methyl-anilino]pyrido[3,2-d]pyrimidin-6-yl]piperazine-1-carboxylate (92 mg, 73% yield) as a yellow oil. LCMS (MM-ES+APCI, Pos): m/z 604.4 (M+H).

Step B. N-(2-fluoro-4-((7-fluoro-1-methyl-1H-benzo[d][1,2,3]triazol-5-yl)oxy)-3-methylphenyl)-6-(piperazin-1-yl)pyrido[3,2-d]pyrimidin-4-amine 2,2,2-trifluoroacetate. Prepared according to Example 68 Step B, substituting tert-butyl 4-[4-[2-fluoro-4-(7-fluoro-1-methyl-benzotriazol-5-yl)oxy-3-methyl-anilino]pyrido[3,2-d]pyrimidin-6-yl]piperazine-1-carboxylate for of tert-butyl 3-(4-((3-methyl-4-((1-methyl-1H-benzo[d][1,2,3]triazol-5-yl)oxy)phenyl)amino)pyrido[3,2-d]pyrimidin-6-yl)-3,8-diazabicyclo[3.2.1]octane-8-carboxylate to afford N-[2-fluoro-4-(7-fluoro-1-methyl-benzotriazol-5-yl)oxy-3-methyl-phenyl]-6-piperazin-1-yl-pyrido[3,2-d]pyrimidin-4-amine 2,2,2-trifluoroacetate (94 mg, quant. yield) as a yellow oil. LCMS (MM-ES+APCI, Pos): m/z 504.2 (M+H-TFA).

Step C. 1-(4-(4-((2-fluoro-4-((7-fluoro-1-methyl-1H-benzo[d][1,2,3]triazol-5-yl)oxy)-3-methylphenyl)amino)pyrido[3,2-d]pyrimidin-6-yl)piperazin-1-yl)prop-2-en-1-one. Prepared according to Example 68 Step C, substituting N-[2-fluoro-4-(7-fluoro-1-methyl-benzotriazol-5-yl)oxy-3-methyl-phenyl]-6-piperazin-1-yl-pyrido[3,2-d]pyrimidin-4-amine 2,2,2-trifluoroacetate (76 mg, 0.12 mmol) for 6-(3,8-diazabicyclo[3.2.1]octan-3-yl)-N-(3-methyl-4-((1-methyl-1H-benzo[d][1,2,3]triazol-5-yl)oxy)phenyl)pyrido[3,2-d]pyrimidin-4-amine to give 1-[4-[4-[2-fluoro-4-(7-fluoro-1-methyl-benzotriazol-5-yl)oxy-3-methyl-anilino]pyrido[3,2-d]pyrimidin-6-yl]piperazin-1-yl]prop-2-en-1-one (4.7 mg, 7% yield) as an off-white solid. LCMS (MM-ES+APCI, Pos): m/z 558.3 (M+H).

### Example 80

### 1-(4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one (compound 92)

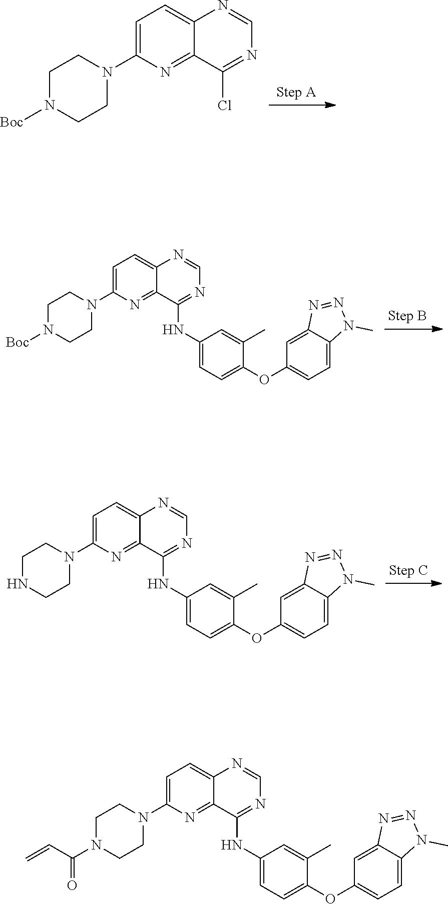

Step A. tert-butyl 4-(8-((3-methyl-4-((1-methyl-1H-benzo[d][1,2,3]triazol-5-yl)oxy)phenyl)amino)pyrimido[5,4-d]pyrimidin-2-yl)piperazine-1-carboxylate. To a solution of tert-butyl 4-(4-chloropyrido[3,2-d]pyrimidin-6-yl)piperazine-1-carboxylate (310 mg, 0.88 mmol) in IPA (5 mL) was added 3-methyl-4-(1-methylbenzotriazol-5-yl)oxy-aniline (152 mg, 0.59 mmol). The mixture was stirred at 80° C. for 2 h, quenched with H₂O (10 mL), and extracted with EtOAc (10 mL×3). The combined organic layers were washed with brine (10 mL), dried over anhydrous Na₂SO₄, filtered, and concentrated under reduced pressure. The crude residue was purified by preparative HPLC (45% to 75% MeCN/10 mM aqueous NH₄HCO₃) to provide tert-butyl 4-[4-[3-methyl-4-(1-methylbenzotriazol-5-yl)oxy-anilino]pyrido[3,2-d]pyrimidin-6-yl]piperazine-1-carboxylate (150 mg, 43% yield) as a white solid. LCMS (MM-ES+APCI, Pos): m/z 568.1 (M+H).

Step B. N-(3-methyl-4-((1-methyl-1H-benzo[d][1,2,3]triazol-5-yl)oxy)phenyl)-6-(piperazin-1-yl)pyrimido[5,4-d]pyrimidin-4-amine 2,2,2-trifluoroacetate. To a solution of tert-butyl 4-[4-[3-methyl-4-(1-methylbenzotriazol-5-yl)oxy-anilino]pyrido[3,2-d]pyrimidin-6-yl]piperazine-1-carboxylate (140 mg, 0.24 mmol) in DCM (2 mL) was added TFA (2 mL, 27.0 mmol). The mixture was stirred at 25° C. for 1 h and concentrated in vacuo to provide N-[3-methyl-4-(1-methylbenzotriazol-5-yl)oxy-phenyl]-6-piperazin-1-yl-pyrido[3,2-d]pyrimidin-4-amine 2,2,2-trifluoroacetate (140 mg, quant. yield) as a yellow oil LCMS (MM-ES+APCI, Pos): m/z 468.1 (M+H-TFA).

Step C. 1-(4-(8-((3-methyl-4-((1-methyl-1H-benzo[d][1,2,3]triazol-5-yl)oxy)phenyl)amino)pyrimido[5,4-d]pyrimidin-2-yl)piperazin-1-yl)prop-2-en-1-one. A solution of N-[3-methyl-4-(1-methylbenzotriazol-5-yl)oxy-phenyl]-6-piperazin-1-yl-pyrido[3,2-d]pyrimidin-4-amine 2,2,2-trifluoroacetate (140 mg, 0.24 mmol) in DCM (3 mL) was purged with N₂ and cooled to −40° C. TEA (101 μL, 0.72 mmol) and prop-2-enoyl chloride (24 μL, 0.29 mmol) were added dropwise and the mixture was stirred at −40° C. for 1 h. The reaction was quenched with sat. aq. NaHCO₃ (5 mL) and extracted with EtOAc (5 mL×3). The combined organic layers were washed with brine (5 mL), dried over anhydrous Na₂SO₄, filtered, and concentrated under reduced pressure. The crude material was purified by preparative HPLC (25% to 55% MeCN/10 mM aqueous NH₄HCO₃) to afford 1-[4-[4-[3-methyl-4-(1-methylbenzotriazol-5-yl)oxy-anilino]pyrido[3,2-d]pyrimidin-6-yl]piperazin-1-yl]prop-2-en-1-one (50 mg, 39% yield) as a yellow solid. LCMS (MM-ES+APCI, Pos): m/z 522.1 (M+H).

Compounds 92, 370-372 in Table 24 below were synthesized according to Example 80 steps A to C.

TABLE 24
Compounds 92, 370-372

| | | | MS |
|---|---|---|---|
| Cmp. | | | API |
| ID | Structure | Chemical Name | (m/z) |
| 92 | 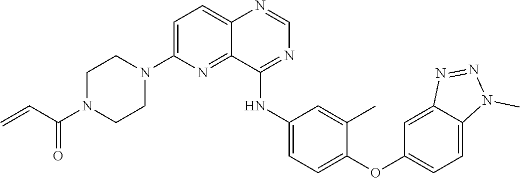 [SMILES: C=CC(=O)N1CCN(c2ccc3ncnc(Nc4ccc(Oc5ccc6c(c5)nnn6C)c(C)c4)c3n2)CC1] | 1-(4-(4-((3-methyl-4-((1-methyl-1H- benzo[d][1,2,3]triazol-5- yl)oxy)phenyl)amino)pyrido[3,2- d]pyrimidin-6-yl)piperazin-1- yl)prop-2-en-1-one | 522.1 (M + H) |
| 370 | 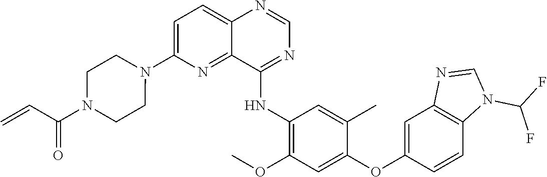 [SMILES: C=CC(=O)N1CCN(c2ccc3ncnc(Nc4cc(C)c(Oc5ccc6c(c5)ncn6C(F)F)cc4OC)c3n2)CC1] | 1-(4-(4-((4-((1-(difluoromethyl)-1H- benzo[d]imidazol-5-yl)oxy)-2- methoxy-5- methylphenyl)amino)pyrido[3,2- d]pyrimidin-6-yl)piperazin-1- yl)prop-2-en-1-one | 587.3 (M + H) |
| 371 | 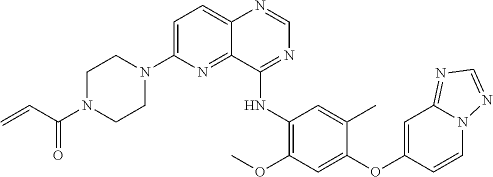 [SMILES: C=CC(=O)N1CCN(c2ccc3ncnc(Nc4cc(C)c(Oc5ccn6ncnc6c5)cc4OC)c3n2)CC1] | 1-(4-(4-((4-([1,2,4]triazolo[1,5- a]pyridin-7-yloxy)-2-methoxy-5- methylphenyl)amino)pyrido[3,2- d]pyrimidin-6-yl)piperazin-1- yl)prop-2-en-1-one | 538.2 (M + H) |
| 372 | 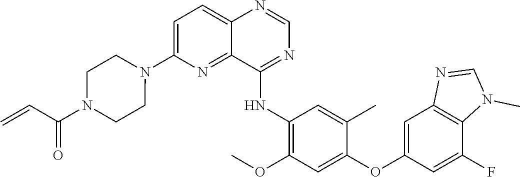 [SMILES: C=CC(=O)N1CCN(c2ccc3ncnc(Nc4cc(C)c(Oc5cc(F)c6c(c5)ncn6C)cc4OC)c3n2)CC1] | 1-(4-(4-((4-((7-fluoro-1-methyl-1H- benzo[d]imidazol-5-yl)oxy)-2- methoxy-5- methylphenyl)amino)pyrido[3,2- d]pyrimidin-6-yl)piperazin-1- yl)prop-2-en-1-one | 569.3 (M + H) |

### Example 81

### 1-(3-(4-((3-methyl-4-((1-methyl-1H-benzo[d][1,2,3]triazol-5-yl)oxy)phenyl)amino)pyrido[3,2-d]pyrimidin-6-yl)-3,6-diazabicyclo[3.1.1]heptan-6-yl)prop-2-en-1-one (compound 93)

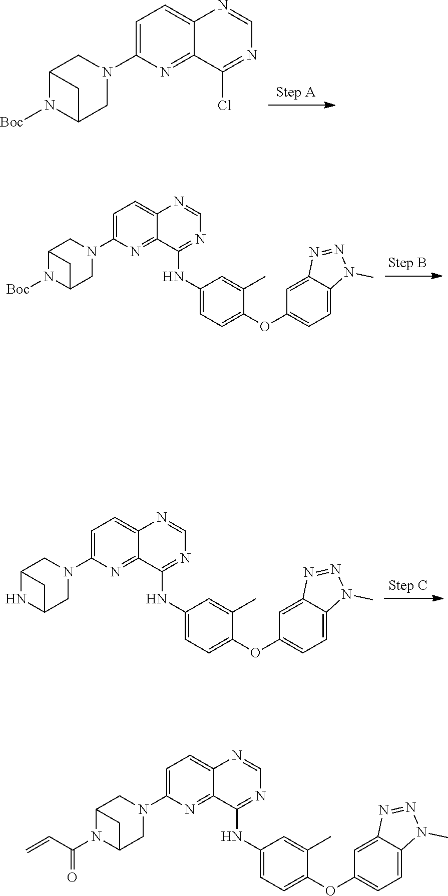

1-(3-(4-((3-methyl-4-((1-methyl-1H-benzo[d][1,2,3]triazol-5-yl)oxy)phenyl)amino)pyrido[3,2-d]pyrimidin-6-yl)-3,6-diazabicyclo[3.1.1]heptan-6-yl)prop-2-en-1-one. Prepared according to Example 80 Steps A to C, substituting of tert-butyl 3-(4-chloropyrido[3,2-d]pyrimidin-6-yl)-3,6-diazabicyclo[3.1.1]heptane-6-carboxylate for tert-butyl 4-(4-chloropyrido[3,2-d]pyrimidin-6-yl)piperazine-1-carboxylate to give 1-[3-[4-[3-methyl-4-(1-methylbenzotriazol-5-yl)oxy-anilino]pyrido[3,2-d]pyrimidin-6-yl]-3,6-diazabicyclo[3.1.1]heptan-6-yl]prop-2-en-1-one (22 mg, 6% yield) as a yellow solid. LCMS (MM-ES+APCI, Pos): m/z 534.4 (M+H).

### Example 82

### 1-(4-(4-((2-fluoro-5-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (compound 94)

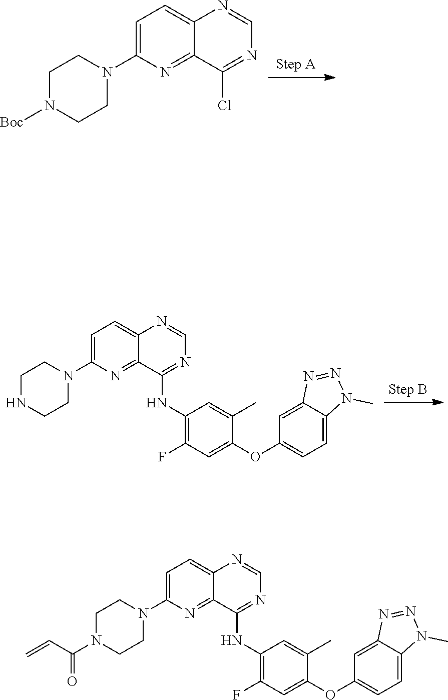

Step A. N-(2-fluoro-5-methyl-4-((1-methyl-H-benzo[d][1,2,3]triazol-5-yl)oxy)phenyl)-6-(piperazin-1-yl)pyrido[3,2-d]pyrimidin-4-amine. Prepared according to Example 80 Step A, substituting 2-fluoro-5-methyl-4-(1-methylbenzotriazol-5-yl)oxy-aniline for 3-methyl-4-(1-methylbenzotriazol-5-yl)oxy-aniline to provide N-[2-fluoro-5-methyl-4-(1-methylbenzotriazol-5-yl)oxy-phenyl]-6-piperazin-1-yl-pyrido[3,2-d]pyrimidin-4-amine (150 mg, 77%) as a light yellow solid. LCMS (MM-ES+APCI, Pos): m/z 486.2 (M+H).

Step B. 1-(4-(4-((2-fluoro-5-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one. Prepared according to Example 80 Step C, substituting N-[2-fluoro-5-methyl-4-(1-methylbenzotriazol-5-yl)oxy-phenyl]-6-piperazin-1-yl-pyrido[3,2-d]pyrimidin-4-amine for tert-butyl 4-[4-1[3-methyl-4-(1-methylbenzotriazol-5-yl)oxy-anilino]pyrido[3,2-d]pyrimidin-6-yl]piperazine-1-carboxylate to yield 1-[4-[4-[2-fluoro-5-methyl-4-(1-methylbenzotriazol-5-yl)oxy-anilino]pyrido[3,2-d]pyrimidin-6-yl]piperazin-1-yl]prop-2-en-1-one (70 mg, 46% yield) as a white solid. LCMS (MM-ES+APCI, Pos): m/z 540.3 (M+H).

### Example 83

### 1-(4-(4-((2-fluoro-3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (compound 95)

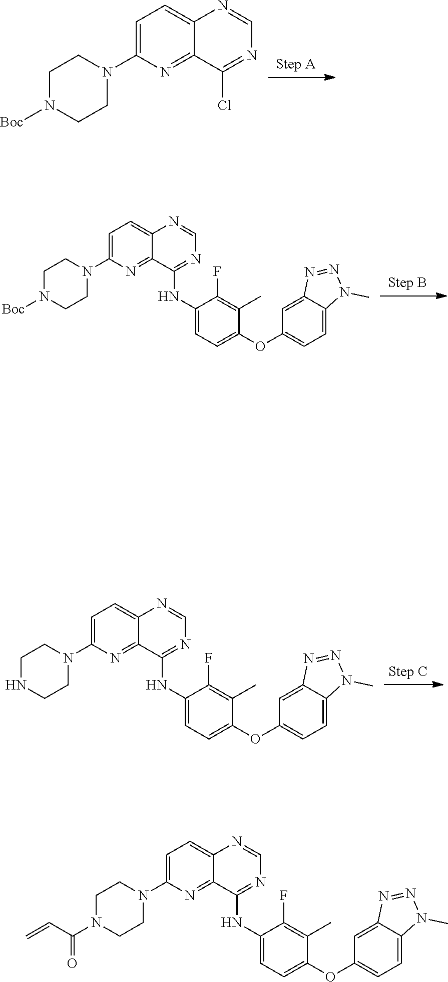

Step A. tert-butyl 4-(4-((2-fluoro-3-methyl-4-((1-methyl-1H-benzo[d][1,2,3]triazol-5-yl)oxy)phenyl)amino)pyrido[3,2-d]pyrimidin-6-yl)piperazine-1-carboxylate. To a solution of 2-fluoro-3-methyl-4-(1-methylbenzotriazol-5-yl)oxy-aniline (100 mg, 0.37 mmol) in IPA (6 mL) was added DIPEA (0.19 mL, 1.10 mmol) and tert-butyl 4-(4-chloropyrido[3,2-d]pyrimidin-6-yl)piperazine-1-carboxylate (193 mg, 0.55 mmol). The reaction was stirred at 90° C. for 2 h, diluted with water (20 mL), and extracted with 10% MeOH/DCM (20 mL×3). The combined organic layers were washed with brine (30 mL), dried over anhydrous Na₂SO₄, filtered, and concentrated under reduced pressure. The crude solid was purified by silica gel column chromatography (0% to 75% EtOAc/PET) to afford tert-butyl 4-[4-[2-fluoro-3-methyl-4-(1-methylbenzotriazol-5-yl)oxy-anilino]pyrido[3,2-d]pyrimidin-6-yl]piperazine-1-carboxylate (100 mg, 35% yield) as a yellow solid. LCMS (MM-ES+APCI, Pos): m/z 586.3 (M+H).

Step B. N-(2-fluoro-3-methyl-4-((1-methyl-H-benzo[d][1,2,3]triazol-5-yl)oxy)phenyl)-6-(piperazin-1-yl)pyrido[3,2-d]pyrimidin-4-amine hydrochloride. To a solution of tert-butyl 4-[4-[2-fluoro-3-methyl-4-(1-methylbenzotriazol-5-yl)oxy-anilino]pyrido[3,2-d]pyrimidin-6-yl]piperazine-1-carboxylate (100 mg, 0.13 mmol) in DCM (2.5 mL) was added HCl (4M in 1,4-dioxane, 2.5 mL, 10 mmol). The mixture was stirred at 25° C. for 1 h and concentrated in vacuo to afford N-[2-fluoro-3-methyl-4-(1-methylbenzotriazol-5-yl)oxy-phenyl]-6-piperazin-1-yl-pyrido[3,2-d]pyrimidin-4-amine hydrochloride (105 mg, quant.) as a yellow solid. LCMS (MM-ES+APCI, Pos): m/z 486.2 (M+H—HCl).

Step C. 1-(4-(4-((2-fluoro-3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one. To a solution of N-[2-fluoro-3-methyl-4-(1-methylbenzotriazol-5-yl)oxy-phenyl]-6-piperazin-1-yl-pyrido[3,2-d]pyrimidin-4-amine hydrochloride (100 mg, 0.19 mmol) in DMF (8 mL) was added DIPEA (0.10 mL, 0.57 mmol). The solution was cooled to −50° C., followed by dropwise addition of prop-2-enoyl chloride (16 μL, 0.19 mmol) in DMF (0.5 mL). The mixture was stirred for 1 h at −50° C. and quenched with sat. aq. NaHCO₃ (0.5 mL). The crude material was purified by preparative HPLC (24% to 54% MeCN/0.08% aqueous NH₄HCO₃) to afford 1-[4-[4-[2-fluoro-3-methyl-4-(1-methylbenzotriazol-5-yl)oxy-anilino]pyrido[3,2-d]pyrimidin-6-yl]piperazin-1-yl]prop-2-en-1-one (49 mg, 46% yield) as a yellow solid. LCMS (MM-ES+APCI, Pos): m/z 540.3 (M+H).

### Example 84

### 1-(6-(4-((3-methyl-4-((1-methyl-1H-benzo[d][1,2,3]triazol-5-yl)oxy)phenyl)amino)pyrido[3,2-d]pyrimidin-6-yl)-3,6-diazabicyclo[3.1.1]heptan-3-yl)prop-2-en-1-one (compound 96)

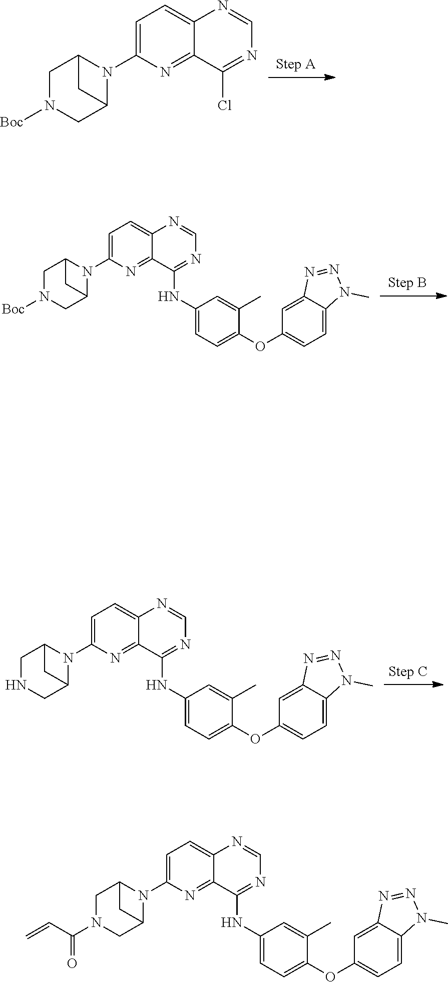

Step A. tert-butyl 6-(4-((3-methyl-4-((1-methyl-1H-benzo[d][1,2,3]triazol-5-yl)oxy)phenyl)amino)pyrido[3,2-d]pyrimidin-6-yl)-3,6-diazabicyclo[3.1.1]heptane-3-carboxylate. To a solution of tert-butyl 6-(4-chloropyrido[3,2-d]pyrimidin-6-yl)-3,6-diazabicyclo[3.1.1]heptane-3-carboxylate (410 mg, 1.13 mmol) in IPA (3 mL) were added 3-methyl-4-(1-methylbenzotriazol-5-yl)oxy-aniline (288 mg, 1.13 mmol) and DIPEA (0.59 mL, 3.40 mmol). The mixture was stirred at 80° C. for 8 h and concentrated in vacuo. The crude residue was purified by silica gel column chromatography (5% to 10% MeOH/DCM) to give tert-butyl 6-[4-[3-methyl-4-(1-methylbenzotriazol-5-yl)oxy-anilino]pyrido[3,2-d]pyrimidin-6-yl]-3,6-diazabicyclo[3.1.1]heptane-3-carboxylate (140 mg, 21% yield) as a white solid. LCMS (MM-ES+APCI, Pos): m/z 580.3 (M+H).

Step B. 6-(3,6-diazabicyclo[3.1.1]heptan-6-yl)-N-(3-methyl-4-((1-methyl-1H-benzo[d][1,2,3]triazol-5-yl)oxy)phenyl)pyrido[3,2-d]pyrimidin-4-amine. To a solution of tert-butyl 6-[4-[3-methyl-4-(1-methylbenzotriazol-5-yl)oxy-anilino]pyrido[3,2-d]pyrimidin-6-yl]-3,6-diazabicyclo[3.1.1]heptane-3-carboxylate (120 mg, 0.21 mmol) in DCM (2 mL) was added TFA (2 mL, 27 mmol). The mixture was stirred at 25° C. for 1 h, quenched slowly with sat. aq. NaHCO₃ (10 mL), and extracted with EtOAc (10 mL×3). The combined organic layers were washed with brine (10 mL), dried over anhydrous Na₂SO₄, filtered, and concentrated under reduced pressure. The crude residue was purified by preparative TLC (10% MeOH/DCM) to afford 6-(3,6-diazabicyclo[3.1.1]heptan-6-yl)-N-[3-methyl-4-(1-methylbenzotriazol-5-yl)oxy-phenyl]pyrido[3,2-d]pyrimidin-4-amine (60 mg, 58% yield) as a yellow solid. LCMS (MM-ES+APCI, Pos): m/z 480.1 (M+H).

Step C. 1-(6-(4-((3-methyl-4-((1-methyl-1H-benzo[d][1,2,3]triazol-5-yl)oxy)phenyl)amino)pyrido[3,2-d]pyrimidin-6-yl)-3,6-diazabicyclo[3.1.1]heptan-3-yl)prop-2-en-I-one. A solution of 6-(3,6-diazabicyclo[3.1.1]heptan-6-yl)-N-[3-methyl-4-(1-methylbenzotriazol-5-yl)oxy-phenyl]pyrido[3,2-d]pyrimidin-4-amine (50 mg, 0.10 mmol) in DCM (2 mL) was placed under N₂ and cooled to 0° C. TEA (42 μL, 0.30 mmol) and prop-2-enoyl chloride (9.8 μL, 0.12 mmol) were added dropwise. The mixture was stirred at 0° C. for 1 h, quenched with sat. aq. NaHCO₃ (10 mL) and extracted with EtOAc (10 mL×3). The combined organic layers were washed with brine (10 mL), dried over anhydrous Na₂SO₄, filtered, and concentrated under reduced pressure. The crude residue was purified by preparative HPLC (27% to 57% MeCN/0.08% aqueous NH₄HCO₃) to afford 1-[6-[4-[3-methyl-4-(1-methylbenzotriazol-5-yl)oxy-anilino]pyrido[3,2-d]pyrimidin-6-yl]-3,6-diazabicyclo[3.1.1]heptan-3-yl]prop-2-en-1-one (12 mg, 22% yield) as a white solid. LCMS (MM-ES+APCI, Pos): m/z 534.2 (M+H).

### Example 85

### 1-(4-(4-((4-((7-fluoro-1-methyl-1H-benzo[d][1,2,3]triazol-5-yl)oxy)-3-methylphenyl)amino)pyrido[3,2-d]pyrimidin-6-yl)piperazin-1-yl)prop-2-en-1-one

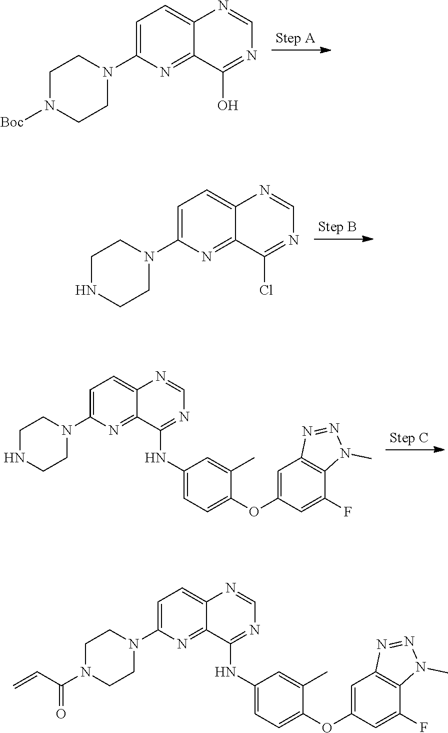

Step A. 4-chloro-6-(piperazin-1-yl)pyrido[3,2-d]pyrimidine. To a solution of tert-butyl 4-(4-hydroxypyrido[3,2-d]pyrimidin-6-yl)piperazine-1-carboxylate (120 mg, 0.36 mmol) in toluene (1.2 mL) were added DIPEA (0.13 mL, 0.72 mmol) and POCl₃ (0.34 mL, 3.62 mmol) at ambient temperature. The mixture was heated to 100° C., stirred for 1 h, and concentrated under reduced pressure to afford 4-chloro-6-(piperazin-1-yl)pyrido[3,2-d]pyrimidine (90 mg, 20% purity) as a brown solid. LCMS (MM-ES+APCI, Pos): m/z 250.0 (M+H).

Step B. N-(4-((7-fluoro-1-methyl-1H-benzo[d][1,2,3]triazol-5-yl)oxy)-3-methylphenyl)-6-(piperazin-1-yl)pyrido[3,2-d]pyrimidin-4-amine. To a solution of 4-chloro-6-(piperazin-1-yl)pyrido[3,2-d]pyrimidine (86 mg, 0.34 mmol) in IPA (2.0 mL) was added 4-((7-fluoro-1-methyl-1H-benzo[d][1,2,3]triazol-5-yl)oxy)-3-methylaniline (56 mg, 0.21 mmol). The reaction was stirred at 80° C. for 1 h and concentrated in vacuo. The residue was diluted with water (50 mL), basified with sat. aq. NaHCO₃ (pH 8) and extracted with DCM (50 mL×3). The combined organic layers were dried over anhydrous Na₂SO₄, filtered and concentrated under reduced pressure. The crude material was purified by silica gel column chromatography (1% to 40% MeOH/DCM) to afford N-(4-((7-fluoro-1-methyl-1H-benzo[d][1,2,3]triazol-5-yl)oxy)-3-methylphenyl)-6-(piperazin-1-yl)pyrido[3,2-d]pyrimidin-4-amine (70 mg, 60% yield) as a yellow solid. LCMS (MM-ES+APCI, Pos): m/z 486.4 (M+H).

Step C. 1-(4-(4-((4-((7-fluoro-1-methyl-1H-benzo[d][1,2,3]triazol-5-yl)oxy)-3-methylphenyl)amino)pyrido[3,2-d]pyrimidin-6-yl)piperazin-1-yl)prop-2-en-1-one. To a solution of N-(4-((7-fluoro-1-methyl-1H-benzo[d][1,2,3]triazol-5-yl)oxy)-3-methylphenyl)-6-(piperazin-1-yl)pyrido[3,2-d]pyrimidin-4-amine (50 mg, 0.10 mmol) in DCM (1.0 mL) was added TEA (43 μL, 0.31 mmol). The mixture was cooled to 0° C. and prop-2-enoyl chloride (8 μL, 0.10 mmol) was added dropwise. The reaction was stirred at 0° C. for 0.5 h, and concentrated in vacuo. The crude solid was purified by preparative HPLC (30% to 60% MeCN/10 mM aqueous NH₄HCO₃) to afford 1-(4-(4-((4-((7-fluoro-1-methyl-1H-benzo[d][1,2,3]triazol-5-yl)oxy)-3-methylphenyl) amino)pyrido[3,2-d]pyrimidin-6-yl)piperazin-1-yl)prop-2-en-1-one (12 mg, 19% yield) as a yellow solid. LCMS (MM-ES+APCI, Pos): m/z 540.3 (M+H).

### Example 86

### 1-(4-(7-methoxy-4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (compound 98)

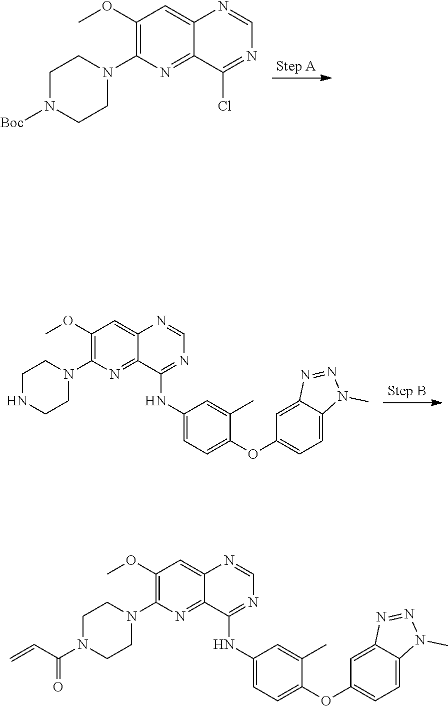

Step A. 7-methoxy-N-(3-methyl-4-((1-methyl-1H-benzo[d][1,2,3]triazol-5-yl)oxy)phenyl)-6-(piperazin-1-yl)pyrido[3,2-d]pyrimidin-4-amine. To a solution of tert-butyl 4-(4-chloro-7-methoxy-pyrido[3,2-d]pyrimidin-6-yl)piperazine-1-carboxylate hydrochloride (157 mg, 0.38 mmol) in IPA (2 mL) was added 3-methyl-4-(1-methylbenzotriazol-5-yl)oxy-aniline (58 mg, 0.23 mmol). The reaction was stirred at 80° C. for 1 h and neutralized (pH 7) with sat. aq. NaHCO₃. The mixture was concentrated in vacuo and the residue was purified by silica gel column chromatography (20% to 30% MeOH/DCM) to provide 7-methoxy-N-[3-methyl-4-(1-methylbenzotriazol-5-yl)oxy-phenyl]-6-piperazin-1-yl-pyrido[3,2-d]pyrimidin-4-amine (125 mg, quant. yield) as a yellow oil. LCMS (MM-ES+APCI, Pos): m/z 498.3 (M+H).

Step B. 1-(4-(7-methoxy-4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one. To a solution of 7-methoxy-N-[3-methyl-4-(1-methylbenzotriazol-5-yl)oxy-phenyl]-6-piperazin-1-yl-pyrido[3,2-d]pyrimidin-4-amine (125 mg, 0.25 mmol) in DCM (15 mL) was added TEA (0.10 mL, 0.75 mmol). The reaction was cooled to 0° C. and prop-2-enoyl chloride (41 μL, 0.50 mmol) was added. After stirring at 0° C. for 30 min, the mixture was concentrated in vacuo. The crude residue was purified by preparative HPLC (28% to 58% MeCN/10 mM aqueous NH₄HCO₃) to afford 1-[4-[7-methoxy-4-[3-methyl-4-(1-methylbenzotriazol-5-yl)oxy-anilino]pyrido[3,2-d]pyrimidin-6-yl]piperazin-1-yl]prop-2-en-1-one (38.5 mg, 28% yield) as a yellow solid. LCMS (MM-ES+APCI, Pos): m/z 552.2 (M+H).

### Example 87

### 1-(4-(4-((2-fluoro-4-((7-fluoro-1-methyl-1H-benzo[d][1,2,3]triazol-5-yl)oxy)-3-methylphenyl)amino)-7-methoxypyrido[3,2-d]pyrimidin-6-yl)piperazin-1-yl)prop-2-en-1-one (compound 99)

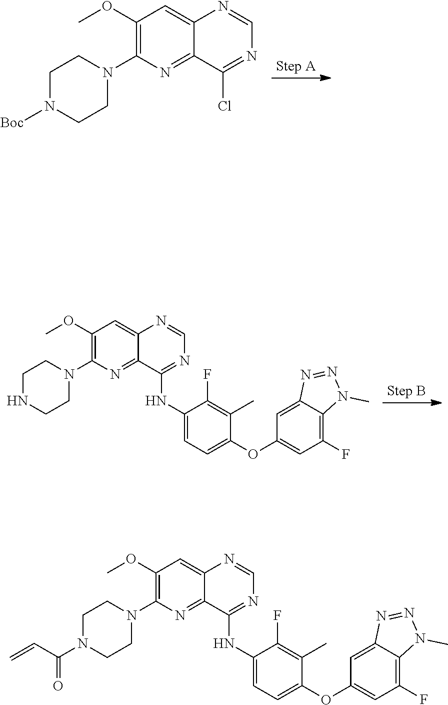

1-(4-(4-((2-fluoro-4-((7-fluoro-1-methyl-1H-benzo[d][1,2,3]triazol-5-yl)oxy)-3-methylphenyl)amino)-7-methoxypyrido[3,2-d]pyrimidin-6-yl)piperazin-1-yl)prop-2-en-1-one. Prepared according to Example 86 Steps A to B, substituting 2-fluoro-4-(7-fluoro-1-methyl-benzotriazol-5-yl)oxy-3-methyl-aniline for 3-methyl-4-(1-methylbenzotriazol-5-yl)oxy-aniline to provide 1-[4-[4-[2-fluoro-4-(7-fluoro-1-methyl-benzotriazol-5-yl)oxy-3-methyl-anilino]-7-methoxy-pyrido[3,2-d]pyrimidin-6-yl]piperazin-1-yl]prop-2-en-1-one (6.5 mg, 6% yield) as a yellow solid. LCMS (MM-ES+APCI, Pos): m/z 588.3 (M+H).

### Example 88

### 1-(4-(7-fluoro-4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (compound 373)

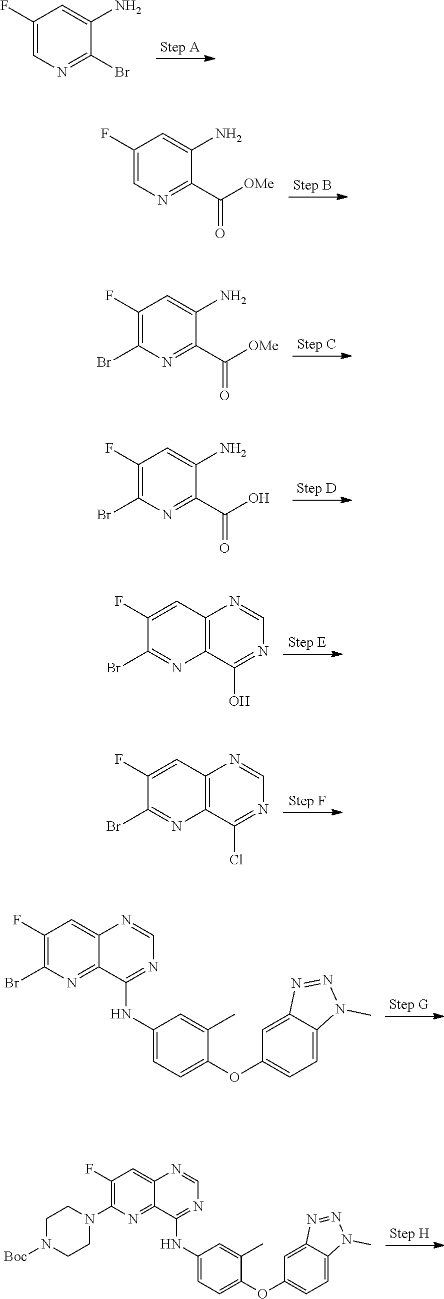

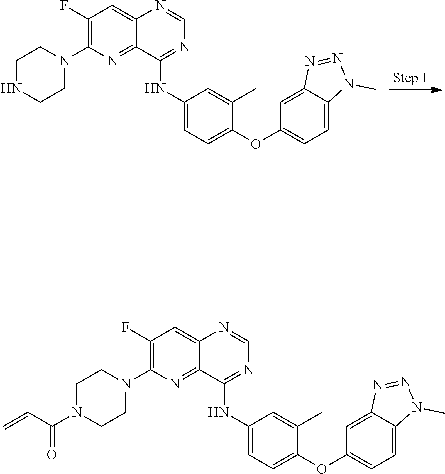

Step A. methyl 3-amino-5-fluoropicolinate. To a solution of 2-bromo-5-fluoro-pyridin-3-amine (2.0 g, 10.5 mmol) in MeOH (50 mL) were added Pd(dppf)Cl₂ (0.38 g 0.52 mmol) and TEA (2.91 mL, 20.9 mmol). The suspension was degassed under vacuum and purged with CO several times. The mixture was heated to 60° C. for 48 h under CO (50 psi). The suspension was filtered through a pad of Celite, eluting with MeOH (50 mL). The filtrate was concentrated under reduced pressure, and the crude product was purified by preparative HPLC (3% to 33% MeCN/0.05% aqueous NH₄HCO₃). Lyophilization afforded methyl 3-amino-5-fluoro-pyridine-2-carboxylate (650 mg, 36% yield) as a white solid. LCMS (MM-ES+APCI, Pos): m/z 171.2 (M+H).

Step B. methyl 3-amino-6-bromo-5-fluoropicolinate. To a solution of methyl 3-amino-5-fluoro-pyridine-2-carboxylate (550 mg, 3.23 mmol) in MeCN (10 mL) was added NBS (690 mg, 3.88 mmol). The mixture was stirred at 25° C. for 2 h. The reaction was diluted with water (30 mL) and extracted with EtOAc (30 mL×3). The combined organic layers were washed with brine (30 mL), dried over anhydrous sodium sulfate, filtered, and concentrated under reduced pressure. The crude product was purified by silica gel column chromatography (20% to 25% EtOAc/PET) to afford methyl 3-amino-6-bromo-5-fluoro-pyridine-2-carboxylate (750 mg, 92% yield) as a white solid. LCMS (MM-ES+APCI, Pos): m/z 249.0, 251.0 (M+H).

Step C. 3-amino-6-bromo-5-fluoropicolinic acid. To a solution of methyl 3-amino-6-bromo-5-fluoro-pyridine-2-carboxylate (300 mg, 1.19 mmol) in THF (2 mL) and water (0.4 mL) was added NaOH (192 mg, 4.80 mmol). The reaction was stirred at 60° C. for 5 h. The mixture was diluted with water (10 mL), acidified with 1M HCl (pH=4). and extracted with EtOAc (10 mL×3). The combined organic layers were washed with brine (10 mL), dried over anhydrous sodium sulfate, filtered, and concentrated under reduced pressure to afford 3-amino-6-bromo-5-fluoropicolinic acid (230 mg, 82% yield) as a gray solid. LCMS (MM-ES+APCI, Pos): m/z 234.9, 236.9 (M+H).

Step D. 6-bromo-7-fluoropyrido[3,2-d]pyrimidin-4-ol. To a solution of 3-amino-6-bromo-5-fluoropicolinic acid (210 mg, 0.89 mmol) in EtOH (3 mL) was added formamidine acetate (200 mg, 1.92 mmol). The reaction was heated in the microwave at 120° C. for 2 h. The mixture was diluted with water (10 mL) and extracted with EtOAc (10 mL×3). The combined organic layers were washed with brine (5 mL), dried over anhydrous sodium sulfate, filtered, and concentrated under reduced pressure to afford 6-bromo-7-fluoro-pyrido[3,2-d]pyrimidin-4-ol (205 mg, 94% yield) as a gray solid. LCMS (MM-ES+APCI, Pos): m/z 244.0, 246.0 (M+H).

Step E. 6-bromo-4-chloro-7-fluoropyrido[3,2-d]pyrimidine. To a solution of 6-bromo-7-fluoro-pyrido[3,2-d]pyrimidin-4-ol (150 mg, 0.61 mmol) in PhMe (3 mL) were added POCl₃ (0.12 mL, 1.24 mmol) and DIPEA (0.54 mL, 3.09 mmol) at 0° C. The reaction was heated to 110° C. for 2 h. The mixture was concentrated under reduced pressure, the crude product was dissolved in EtOAc (10 mL), and sat. aq. NaHCO₃ (5 mL) was added. The layers were separated and the aqueous layer was extracted with EtOAc (10 mL×3). The combined organic layers were washed with brine (5 mL), dried over anhydrous sodium sulfate, filtered, and concentrated under reduced pressure to afford 6-bromo-4-chloro-7-fluoro-pyrido[3,2-d]pyrimidine (180 mg, quant. yield) as a yellow solid.

Step F. 6-bromo-7-fluoro-N-(3-methyl-4-((1-methyl-H-benzo[d][1,2,3]triazol-5-yl)oxy)phenyl)pyrido[3,2-d]pyrimidin-4-amine. To a solution of 6-bromo-4-chloro-7-fluoro-pyrido[3,2-d]pyrimidine (160 mg, 0.61 mmol) in IPA (4 mL) was added 3-methyl-4-(1-methylbenzotriazol-5-yl)oxy-aniline (160 mg, 0.63 mmol). The reaction was stirred at 80° C. for 2 h. The mixture was diluted with water (10 mL) and extracted with EtOAc (10 mL×3). The combined organic layers were washed with brine (10 mL), dried over anhydrous sodium sulfate, filtered, and concentrated under reduced pressure. The crude product was purified by silica gel column chromatography (50% to 65% EtOAc/PET) to provide 6-bromo-7-fluoro-N-[3-methyl-4-(1-methylbenzotriazol-5-yl)oxy-phenyl]pyrido[3,2-d]pyrimidin-4-amine (170 mg, 40% yield) as a yellow solid. LCMS (MM-ES+APCI, Pos): m/z 480.0, 482.0 (M+H).

Step G. tert-butyl 4-(7-fluoro-4-((3-methyl-4-((1-methyl-H-benzo[d][1,2,3]triazol-5-yl)oxy)phenyl)amino)pyrido[3,2-d]pyrimidin-6-yl)piperazine-1-carboxylate. A mixture of tert-butyl piperazine-1-carboxylate (122 mg, 0.66 mmol), 6-bromo-7-fluoro-N-[3-methyl-4-(1-methylbenzotriazol-5-yl)oxy-phenyl]pyrido[3,2-d]pyrimidin-4-amine (150 mg, 0.22 mmol), RuPhos Pd G3 (20 mg, 24 μmol) and Cs₂CO₃ (210 mg, 0.64 mmol) in 1,4-dioxane (3 mL) was degassed and purged with N₂. The reaction was heated to 100° C. for 2.5 h under N₂. The mixture was diluted with water (5 mL) and extracted with EtOAc (10 mL×3). The combined organic layers were washed with brine (5 mL), dried over anhydrous sodium sulfate, filtered, and concentrated under reduced pressure. The crude product was purified by preparative TLC (5% MeOH/DCM) to provide tert-butyl 4-[7-fluoro-4-[3-methyl-4-(1-methylbenzotriazol-5-yl)oxy-anilino]pyrido[3,2-d]pyrimidin-6-yl]piperazine-1-carboxylate (64 mg, 33% yield) as a yellow solid. LCMS (MM-ES+APCI, Pos): m/z 586.3 (M+H).

Step H. 7-fluoro-N-(3-methyl-4-((1-methyl-1H-benzo[d][1,2,3]triazol-5-yl)oxy)phenyl)-6-(piperazin-1-yl)pyrido[3,2-d]pyrimidin-4-amine. To a solution of tert-butyl 4-[7-fluoro-4-[3-methyl-4-(1-methylbenzotriazol-5-yl)oxy-anilino]pyrido[3,2-d]pyrimidin-6-yl]piperazine-1-carboxylate (60 mg, 68 μmol) in DCM (1 mL) was added TFA (1 mL). The reaction was stirred at 25° C. for 2 h and concentrated in vacuo. The mixture was diluted with sat. aq. NaHCO₃ (5 mL) and extracted with EtOAc (10 mL×3). The combined organic layers were washed with brine (5 mL), dried over anhydrous sodium sulfate, filtered, and concentrated under reduced pressure. The crude product was purified by preparative TLC (10% MeOH/DCM) to provide 7-fluoro-N-[3-methyl-4-(1-methylbenzotriazol-5-yl)oxy-phenyl]-6-piperazin-1-yl-pyrido[3,2-d]pyrimidin-4-amine (31 mg, 92% yield) as a yellow solid. LCMS (MM-ES+APCI, Pos): m/z 486.2 (M+H).

Step I. 1-(4-(7-fluoro-4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one. A mixture of 7-fluoro-N-[3-methyl-4-(1-methylbenzotriazol-5-yl)oxy-phenyl]-6-piperazin-1-yl-pyrido[3,2-d]pyrimidin-4-amine (28 mg, 57 μmol) and TEA (28 μL, 198 μmol) in DCM (2 mL) was purged with N₂ and cooled to 0° C. Prop-2-enoyl chloride (5.4 μL, 66 μmol) was added dropwise and the reaction was stirred at 0° C. for 1 h. The mixture was quenched with sat. aq. NaHCO₃ and extracted with EtOAc (10 mL×3). The combined organic layers were washed with brine (5 mL), dried over anhydrous sodium sulfate, filtered, and concentrated under reduced pressure. The crude residue was purified by silica gel column chromatography (7% to 10% MeOH/DCM) to afford 1-[4-[7-fluoro-4-[3-methyl-4-(1-methylbenzotriazol-5-yl)oxy-anilino]pyrido[3,2-d]pyrimidin-6-yl]piperazin-1-yl]prop-2-en-1-one (24 mg, 75% yield) as a yellow solid. LCMS (MM-ES+APCI, Pos): m/z 540.3 (M+H).

### Example 89

### 1-(4-(7-methyl-4-((3-methyl-4-((1-methyl-H-benzo[d][1,2,3]triazol-5-yl)oxy)phenyl)amino)pyrido[3,2-d]pyrimidin-6-yl)piperazin-1-yl)prop-2-en-1-one

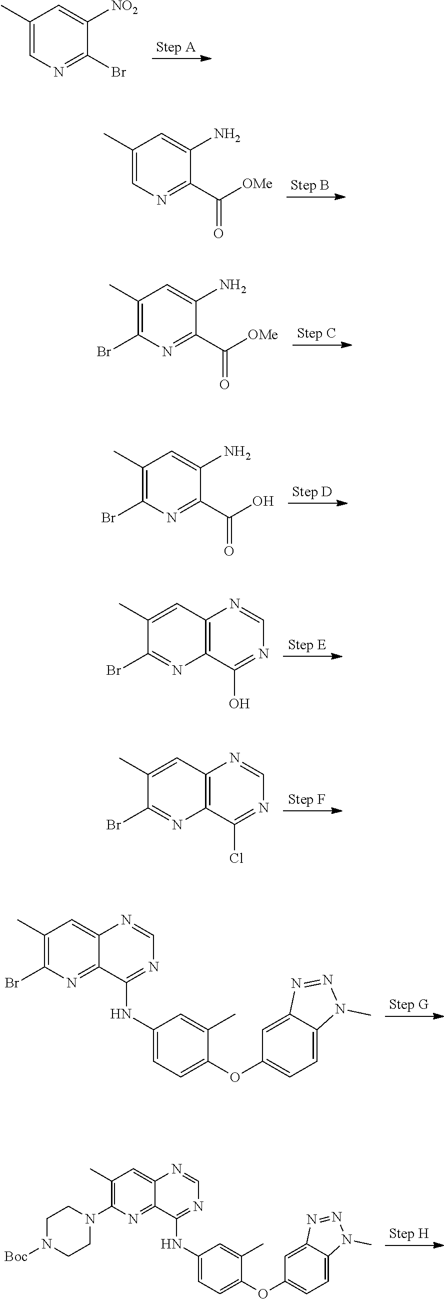

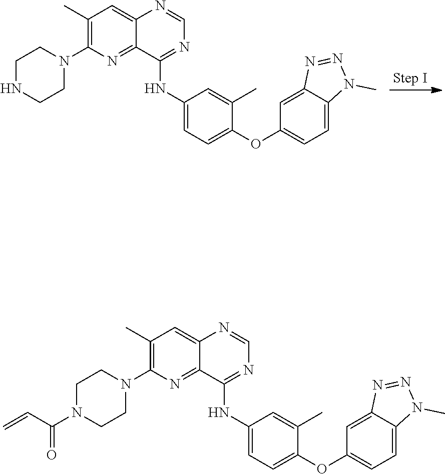

Steps A to D. 6-bromo-7-methylpyrido[3,2-d]pyrimidin-4-ol. Prepared according to Example 88 steps A to D, substituting 2-bromo-5-methyl-3-nitropyridine for 2-bromo-5-fluoro-pyridin-3-amine to afford 6-bromo-7-methylpyrido[3,2-d]pyrimidin-4-ol (200 mg, quant. yield) as a white solid. LCMS (MM-ES+APCI, Pos): m/z 239.8, 241.8 (M+H). Step E. 4,6-dichloro-7-methylpyrido[3,2-d]pyrimidine. To a solution of 6-bromo-7-methyl-pyrido[3,2-d]pyrimidin-4-ol (120 mg, 0.50 mmol) in SOCl₂ (5 mL) was added DMF (4 μL, 0.05 mmol). The mixture was heated at 90° C. for 2 h and concentrated in vacuo to afford 4,6-dichloro-7-methyl-pyrido[3,2-d]pyrimidine (160 mg, quant. yield) as a yellow oil. LCMS (MM-ES+APCI, Pos): m/z 214.1 (M+H).

Step F. 6-chloro-7-methyl-N-(3-methyl-4-((1-methyl-1H-benzo[d][1,2,3]triazol-5-yl)oxy)phenyl)pyrido[3,2-d]pyrimidin-4-amine. Prepared according to Example 88 step F, substituting 4,6-dichloro-7-methyl-pyrido[3,2-d]pyrimidine for 6-bromo-4-chloro-7-fluoro-pyrido[3,2-d]pyrimidine to afford 6-chloro-7-methyl-N-(3-methyl-4-((1-methyl-1H-benzo[d][1,2,3]triazol-5-yl)oxy)phenyl)pyrido[3,2-d]pyrimidin-4-amine (120 mg, 35% yield) as a yellow solid. LCMS (MM-ES+APCI, Pos): m/z 432.2 (M+H).

Step G. tert-butyl 4-(7-methyl-4-((3-methyl-4-((1-methyl-1H-benzo[d][1,2,3]triazol-5-yl)oxy)phenyl)amino)pyrido[3,2-d]pyrimidin-6-yl)piperazine-1-carboxylate. A mixture of 6-chloro-7-methyl-N-[3-methyl-4-(1-methylbenzotriazol-5-yl)oxy-phenyl]pyrido[3,2-d]pyrimidin-4-amine (60 mg, 0.12 mmol), tert-butyl piperazine-1-carboxylate (330 mg, 1.77 mmol), and DIPEA (64 μL, 0.37 mmol) in DMSO (2 mL) was microwaved at 150° C. for 2 h in a sealed tube. The reaction was diluted with water (10 mL) and extracted with EtOAc (10 mL×3). The combined organic layers were washed with brine (10 mL×3), dried over anhydrous sodium sulfate, filtered, and concentrated under reduced pressure. The crude residue was purified by preparative TLC (10% MeOH/DCM) to afford tert-butyl 4-[7-methyl-4-[3-methyl-4-(1-methylbenzotriazol-5-yl)oxy-anilino]pyrido[3,2-d]pyrimidin-6-yl]piperazine-1-carboxylate (40 mg, 53% yield) as a yellow solid. LCMS (MM-ES+APCI, Pos): m/z 582.4 (M+H).

Steps H to I. 1-(4-(7-methyl-4-((3-methyl-4-((1-methyl-H-benzo[d][1,2,3]triazol-5-yl)oxy)phenyl)amino)pyrido[3,2-d]pyrimidin-6-yl)piperazin-1-yl)prop-2-en-1-one. Prepared according to Example 88 steps H to I, substituting tert-butyl 4-[7-methyl-4-[3-methyl-4-(1-methylbenzotriazol-5-yl)oxy-anilino]pyrido[3,2-d]pyrimidin-6-yl]piperazine-1-carboxylate for tert-butyl 4-[7-fluoro-4-[3-methyl-4-(1-methylbenzotriazol-5-yl)oxy-anilino]pyrido[3,2-d]pyrimidin-6-yl]piperazine-1-carboxylate to afford 1-(4-(7-methyl-4-((3-methyl-4-((1-methyl-1H-benzo[d][1,2,3]triazol-5-yl)oxy)phenyl)amino)pyrido[3,2-d]pyrimidin-6-yl)piperazin-1-yl)prop-2-en-1-one (7 mg, 25% yield) as a yellow solid. LCMS (MM-ES+APCI, Pos): m/z 536.3 (M+H).

### Example 90

### 1-(4-(4-((4-([1,2,4]triazolo[1,5-a]pyridin-7-yloxy)-3-methylphenyl)amino)-5-fluoropyrido[3,4-d]pyrimidin-6-yl)piperazin-1-yl)prop-2-en-1-one (compound 100)

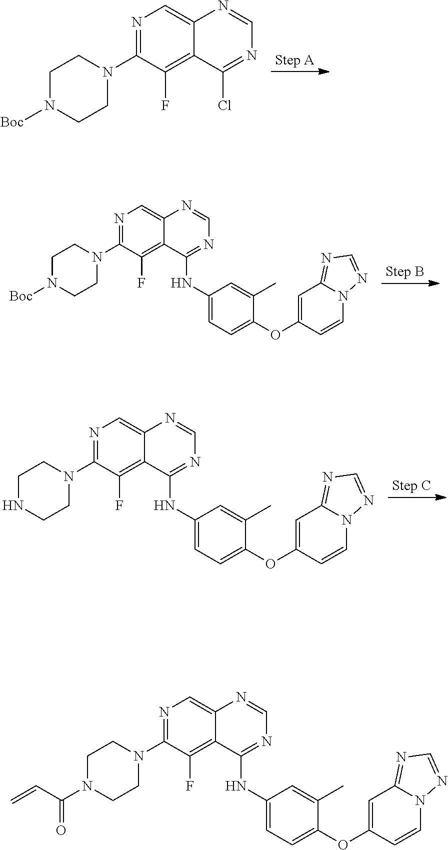 [SMILES: CC(C)(C)OC(=O)N1CCN(c2ncc3ncnc(Cl)c3c2F)CC1 ; Cc1cc(Nc2ncnc3cnc(N4CCN(C(=O)OC(C)(C)C)CC4)c(F)c23)ccc1Oc1ccn2ncnc2c1 ; Cc1cc(Nc2ncnc3cnc(N4CCNCC4)c(F)c23)ccc1Oc1ccn2ncnc2c1 ; C=CC(=O)N1CCN(c2ncc3ncnc(Nc4ccc(Oc5ccn6ncnc6c5)c(C)c4)c3c2F)CC1]

Step A. tert-butyl 4-(4-((4-([1,2,4]triazolo[1,5-a]pyridin-7-yloxy)-3-methylphenyl)amino)-5-fluoropyrido[3,4-d]pyrimidin-6-yl)piperazine-1-carboxylate. To a solution of tert-butyl 4-(4-chloro-5-fluoro-pyrido[3,4-d]pyrimidin-6-yl)piperazine-1-carboxylate (90 mg, 0.24 mmol) in IPA (10 mL) were added DIPEA (0.11 mL, 0.61 mmol) and 3-methyl-4-([1,2,4]triazolo[1,5-a]pyridin-7-yloxy)aniline (49.0 mg, 0.20 mmol). The mixture was stirred for 2 h at 80° C., quenched with water (30 mL), and extracted with EtOAc (40 mL×3). The combined organic layers were washed with brine (30 mL), dried over anhydrous Na₂SO₄, filtered and concentrated under reduced pressure. The crude oil was purified by silica gel column chromatography (1% to 30% EtOAc/PET) to afford tert-butyl 4-[5-fluoro-4-[3-methyl-4-([1,2,4]triazolo[1,5-a]pyridin-7-yloxy)anilino]pyrido[3,4-d]pyrimidin-6-yl]piperazine-1-carboxylate (110 mg, 96% yield) as a yellow solid. LCMS (MM-ES+APCI, Pos): m/z 572.4 (M+H).

Step B. N-(4-([1,2,4]triazolo[1,5-a]pyridin-7-yloxy)-3-methylphenyl)-5-fluoro-6-(piperazin-1-yl)pyrido[3,4-d]pyrimidin-4-amine 2,2,2-trifluoroacetate. To a solution of tert-butyl 4-[5-fluoro-4-[3-methyl-4-([1,2,4]triazolo[1,5-a]pyridin-7-yloxy)anilino]pyrido[3,4-d]pyrimidin-6-yl]piperazine-1-carboxylate (110 mg, 0.19 mmol) in DCM (5 mL) was added TFA (5 mL, 67.3 mmol). The mixture was stirred at 20° C. for 2 h and concentrated in vacuo to afford 5-fluoro-N-[3-methyl-4-([1,2,4]triazolo[1,5-a]pyridin-7-yloxy)phenyl]-6-piperazin-1-yl-pyrido[3,4-d]pyrimidin-4-amine 2,2,2-trifluoroacetate (90 mg, 81% yield) as a yellow solid. LCMS (MM-ES+APCI, Pos): m/z 472.2 (M+H-TFA).

Step C. 1-(4-(4-((4-([1,2,4]triazolo[1,5-a]pyridin-7-yloxy)-3-methylphenyl)amino)-5-fluoropyrido[3,4-d]pyrimidin-6-yl)piperazin-1-yl)prop-2-en-1-one. To a solution of 5-fluoro-N-[3-methyl-4-([1,2,4]triazolo[1,5-a]pyridin-7-yloxy)phenyl]-6-piperazin-1-yl-pyrido[3,4-d]pyrimidin-4-amine 2,2,2-trifluoroacetate (85 mg, 0.15 mmol) in DCM (3 mL) was added DIPEA (94 μL, 0.54 mmol). The mixture was cooled to −78° C., prop-2-enoyl chloride (15 L, 0.18 mmol) was added dropwise, and the reaction was stirred at −78° C. for 5 min. The reaction was quenched with water (10 mL) and extracted with EtOAc (20 mL×3). The combined organic layers were washed with brine (10 mL), dried over anhydrous Na₂SO₄, filtered, and concentrated in vacuo. The crude solid was purified by preparative HPLC (20% to 59% MeCN/0.05% aqueous NH₄OH) to afford 1-(4-(4-((4-([1,2,4]triazolo[1,5-a]pyridin-7-yloxy)-3-methylphenyl)amino)-5-fluoropyrido[3,4-d]pyrimidin-6-yl)piperazin-1-yl)prop-2-en-1-one (38 mg, 40% yield) as a yellow solid. LCMS (MM-ES+APCI, Pos): m/z 526.3 (M+H).

Compounds 100-103 in Table 8 were prepared according to Example 90 steps A to C.

TABLE 8
A list of compounds 101-103

| | | | MS |
|---|---|---|---|
| Cmp. | | | API |
| ID | Structure | Chemical Name | (m/z) |
| 100 | 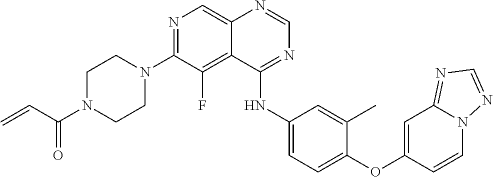 [SMILES: C=CC(=O)N1CCN(c2ncc3ncnc(Nc4ccc(Oc5ccn6ncnc6c5)c(C)c4)c3c2F)CC1] | 1-(4-(4-((4-([1,2,4]triazolo[1,5- a]pyridin-7-yloxy)-3- methylphenyl)amino)-5- fluoropyrido[3,4-d]pyrimidin-6- yl)piperazin-1-yl)prop-2-en-1-one | 526.3 (M + H) |
| 101 | 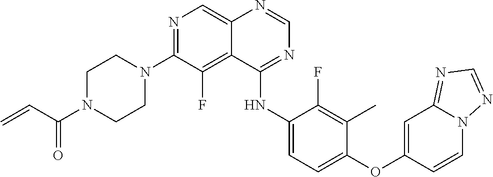 [SMILES: C=CC(=O)N1CCN(c2ncc3ncnc(Nc4ccc(Oc5ccn6ncnc6c5)c(C)c4F)c3c2F)CC1] | 1-(4-(4-((4-([1,2,4]triazolo[1,5- a]pyridin-7-yloxy)-2-fluoro-3- methylphenyl)amino)-5- fluoropyrido[3,4-d]pyrimidin-6- yl)piperazin-1-yl)prop-2-en-1-one | 544.4 (M + H) |
| 102 | 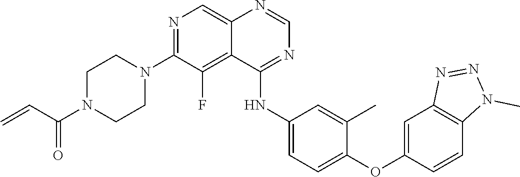 [SMILES: C=CC(=O)N1CCN(c2ncc3ncnc(Nc4ccc(Oc5ccc6c(c5)nnn6C)c(C)c4)c3c2F)CC1] | 1-(4-(5-fluoro-4-((3-methyl-4-((1- methyl-1H-benzo[d][1,2,3]triazol-5- yl)oxy)phenyl)amino)pyrido[3,4- d]pyrimidin-6-yl)piperazin-1-yl)prop- 2-en-1-one | 540.1 (M + H) |
| 103 | 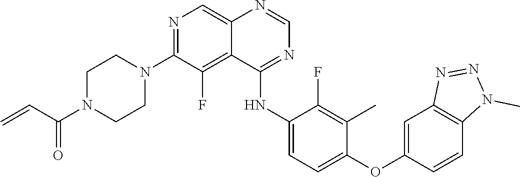 [SMILES: C=CC(=O)N1CCN(c2ncc3ncnc(Nc4ccc(Oc5ccc6c(c5)nnn6C)c(C)c4F)c3c2F)CC1] | 1-(4-(5-fluoro-4-((2-fluoro-3-methyl-4- ((1-methyl-1H-benzo[d][1,2,3]triazol- 5-yl)oxy)phenyl)amino)pyrido[3,4- d]pyrimidin-6-yl)piperazin-1-yl)prop- 2-en-1-one | 558.3 (M + H) |

### Example 91

### 1-(4-(5-fluoro-4-((3-methyl-4-((1-methyl-1H-benzo[d][1,2,3]triazol-5-yl)oxy)phenyl)amino)quinazolin-6-yl)piperazin-1-yl)prop-2-en-1-one (compound 104)

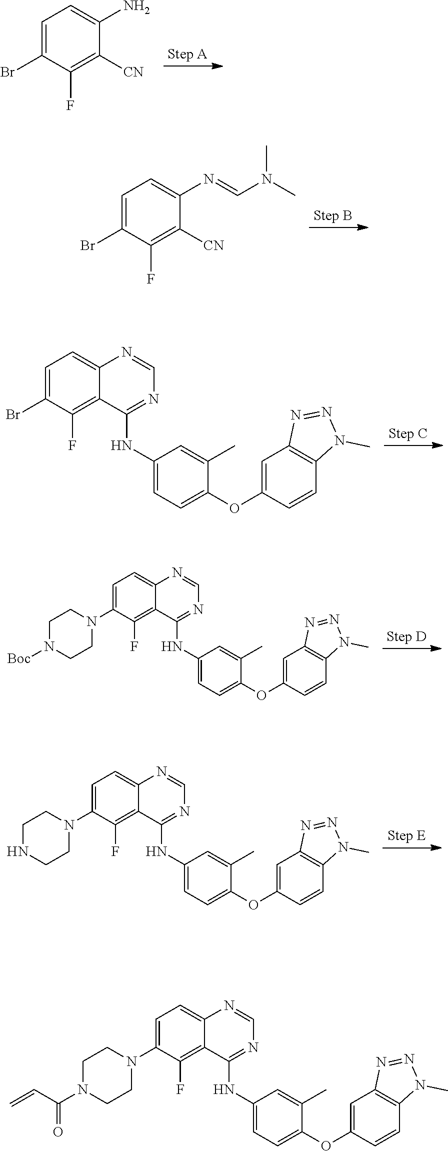

Step A. N′-(4-bromo-2-cyano-3-fluorophenyl)-N,N-dimethylformimidamide. To a solution of 6-amino-3-bromo-2-fluoro-benzonitrile (400 mg, 1.86 mmol) in 1,4-dioxane (4 mL) was added DMF-DMA (0.49 mL, 3.72 mmol). The mixture was heated to 75° C. and stirred for 30 min and concentrated in vacuo to provide N′-(4-bromo-2-cyano-3-fluorophenyl)-N,N-dimethylformimidamide (500 mg, 96% yield) as a red solid. LCMS (MM-ES+APCI, Pos): m/z 270.0 (M+H).

Step B. 6-bromo-5-fluoro-N-(3-methyl-4-((1-methyl-H-benzo[d][1,2,3]triazol-5-yl)oxy)phenyl)quinazolin-4-amine. To a solution of N′-(4-bromo-2-cyano-3-fluorophenyl)-N,N-dimethylformimidamide (300 mg, 1.07 mmol) in AcOH (12 mL) was added 3-methyl-4-((1-methyl-1H-benzo[d][1,2,3]triazol-5-yl)oxy)aniline (274 mg, 1.07 mmol). The mixture was heated to 85° C., stirred for 2 h, and quenched with sat. aq. NaHCO₃. This reaction was combined with another batch (50 mg, 0.17 mmol) and the suspension was filtered. The filter cake was washed with water (3 mL) and the solid was dried in vacuo to provide 6-bromo-5-fluoro-N-(3-methyl-4-((1-methyl-1H-benzo[d][1,2,3]triazol-5-yl)oxy)phenyl)quinazolin-4-amine (415 mg, 70% yield) as an off-white solid. LCMS (MM-ES+APCI, Pos): m/z 479.0 (M+H).

Step C. tert-butyl 4-(5-fluoro-4-((3-methyl-4-((1-methyl-H-benzo[d][1,2,3]triazol-5-yl)oxy)phenyl)amino)quinazolin-6-yl)piperazine-1-carboxylate. To a solution of 6-bromo-5-fluoro-N-(3-methyl-4-((1-methyl-1H-benzo[d][1,2,3]triazol-5-yl)oxy)phenyl)quinazolin-4-amine (175 mg, 0.36 mmol) and tert-butyl piperazine-1-carboxylate (201 mg, 1.08 mmol) in 1,4-dioxane (5 mL) were added Cs₂CO₃ (352 mg, 1.08 mmol), Pd₂(dba)₃ (33 mg, 36 μmol) and Xantphos (42 mg, 72 μmol). The mixture was heated to 100° C., stirred for 4 h, and quenched with water (5 mL). This batch was combined with another quenched batch (200 mg, 0.42 mmol). The aqueous mixture was extracted with EtOAc (5 mL×3), and the combined organic layers were washed with brine (10 mL), dried over anhydrous Na₂SO₄, filtered, and concentrated under reduced pressure. The crude oil was purified by silica gel column chromatography (0% to 80% EtOAc/PET) to afford tert-butyl 4-(5-fluoro-4-((3-methyl-4-((1-methyl-1H-benzo[d][1,2,3]triazol-5-yl)oxy)phenyl)amino)quinazolin-6-yl)piperazine-1-carboxylate (390 mg, 86% yield) as a yellow solid. LCMS (MM-ES+APCI, Pos): m/z 585.4 (M+H).

Step D. 5-fluoro-N-(3-methyl-4-((1-methyl-H-benzo[d][1,2,3]triazol-5-yl)oxy)phenyl)-6-(piperazin-1-yl)quinazolin-4-amine hydrochloride. A solution of tert-butyl 4-(5-fluoro-4-((3-methyl-4-((1-methyl-H-benzo[d][1,2,3]triazol-5-yl)oxy)phenyl)amino)quinazolin-6-yl)piperazine-1-carboxylate (150 mg, 0.23 mmol) in DCM (1 mL) was cooled to 0° C., followed by dropwise addition of HCl (4M in EtOAc, 1 mL, 4.0 mmol). The mixture was stirred at 0° C. for 0.5 h and concentrated in vacuo to afford 5-fluoro-N-(3-methyl-4-((1-methyl-1H-benzo[d][1,2,3]triazol-5-yl)oxy)phenyl)-6-(piperazin-1-yl)quinazolin-4-amine hydrochloride (113 mg, 94% yield) as a yellow solid. LCMS (MM-ES+APCI, Pos): m/z 485.3 (M+H—HCl).

Step E. 1-(4-(5-fluoro-4-((3-methyl-4-((1-methyl-1H-benzo[d][1,2,3]triazol-5-yl)oxy)phenyl)amino)quinazolin-6-yl)piperazin-1-yl)prop-2-en-1-one. Two batches in parallel each contained a solution of 5-fluoro-N-(3-methyl-4-((1-methyl-1H-benzo[d][1,2,3]triazol-5-yl)oxy)phenyl)-6-(piperazin-1-yl)quinazolin-4-amine (50 mg, 91 mol) and DIPEA (79 μL, 453 μmol) in DCM (2 mL). The solution was cooled to −78° C. and prop-2-enoyl chloride (7.4 μL, 91 μmol) was added dropwise. The mixture was stirred at −78° C. for 5 min, quenched with sat. aq. NaHCO₃ (5 mL) and extracted with DCM (5 mL×3). The combined organic layers were washed with brine (15 mL), dried over anhydrous Na₂SO₄, filtered, and concentrated under reduced pressure. The crude residue was purified by preparative HPLC (24% to 44% MeCN/2.25% aqueous TFA) and the two batches were combined to afford 1-(4-(5-fluoro-4-((3-methyl-4-((1-methyl-H-benzo[d][1,2,3]triazol-5-yl)oxy)phenyl)amino)quinazolin-6-yl)piperazin-1-yl)prop-2-en-1-one (34 mg, 35% yield) was obtained as a yellow solid. LCMS (MM-ES+APCI, Pos): m/z 539.3 (M+H).

### Example 92

### 1-(4-(5-methoxy-4-((3-methyl-4-((1-methyl-H-benzo[d][1,2,3]triazol-5-yl)oxy)phenyl)amino)quinazolin-6-yl)piperazin-1-yl)prop-2-en-1-one (compound 105)

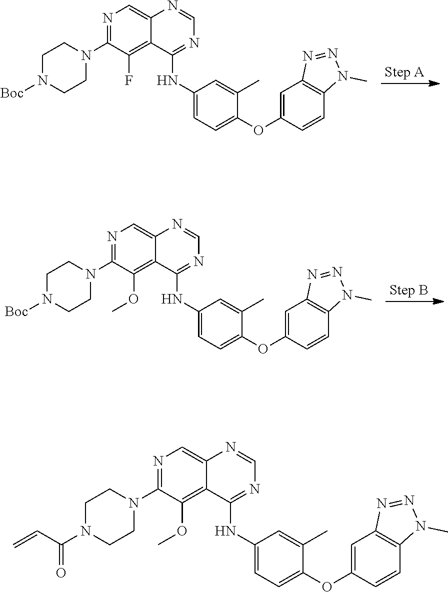

Step A. tert-butyl 4-(5-methoxy-4-((3-methyl-4-((1-methyl-H-benzo[d][1,2,3]triazol-5-yl)oxy)phenyl)amino)quinazolin-6-yl)piperazine-1-carboxylate. To a solution of tert-butyl 4-[5-fluoro-4-[3-methyl-4-(1-methylbenzotriazol-5-yl)oxy-anilino]pyrido[3,4-d]pyrimidin-6-yl]piperazine-1-carboxylate (100 mg, 0.17 mmol) in MeOH (5 mL) was added NaOMe (9.2 mg, 0.17 mmol). The mixture was heated at 70° C. for 2 h and concentrated in vacuo. The crude material was purified by silica gel column chromatography (1% to 50% EtOAc/PET) to afford tert-butyl 4-[5-methoxy-4-[3-methyl-4-(1-methylbenzotriazol-5-yl)oxy-anilino]pyrido[3,4-d]pyrimidin-6-yl]piperazine-1-carboxylate (80 mg, 78% yield) as a yellow solid. LCMS (MM-ES+APCI, Pos): m/z 598.5 (M+H).

Step B. 1-(4-(5-methoxy-4-((3-methyl-4-((1-methyl-H-benzo[d][1,2,3]triazol-5-yl)oxy)phenyl)amino)quinazolin-6-yl)piperazin-1-yl)prop-2-en-1-one. To a solution of tert-butyl 4-[5-methoxy-4-[3-methyl-4-(1-methylbenzotriazol-5-yl)oxy-anilino]pyrido[3,4-d]pyrimidin-6-yl]piperazine-1-carboxylate (70 mg, 0.12 mmol) in DCM (3 mL) was added TFA (3 mL, 40.4 mmol). The mixture was stirred for 2 h at 20° C. and concentrated in vacuo. The crude residue was suspended in DCM (5 mL) and cooled to −78° C. DIPEA (53 μL, 0.30 mmol) and prop-2-enoyl chloride (8.2 μL, 0.10 mmol) were added. The mixture was stirred at −78° C. for 5 min, quenched with water (10 mL), and extracted with EtOAc (20 mL×3). The combined organic layers were washed with brine (10 mL), dried over anhydrous Na₂SO₄, filtered, and concentrated under reduced pressure. The crude solid was purified by preparative HPLC (36% to 66% MeCN/0.05% aqueous NH₄OH) to afford 1-[4-[5-methoxy-4-[3-methyl-4-(1-methylbenzotriazol-5-yl)oxy-anilino]pyrido[3,4-d]pyrimidin-6-yl]piperazin-1-yl]prop-2-en-1-one (18 mg, 32% yield) was obtained as yellow solid. LCMS (MM-ES+APCI, Pos): m/z 552.3 (M+H).

### Example 93

### 1-(4-(4-((4-((1-(difluoromethyl)-1H-benzo[d]imidazol-5-yl)oxy)-2-fluoro-3-methylphenyl)amino)quinazolin-6-yl)piperazin-1-yl)prop-2-en-1-one (compound 375)

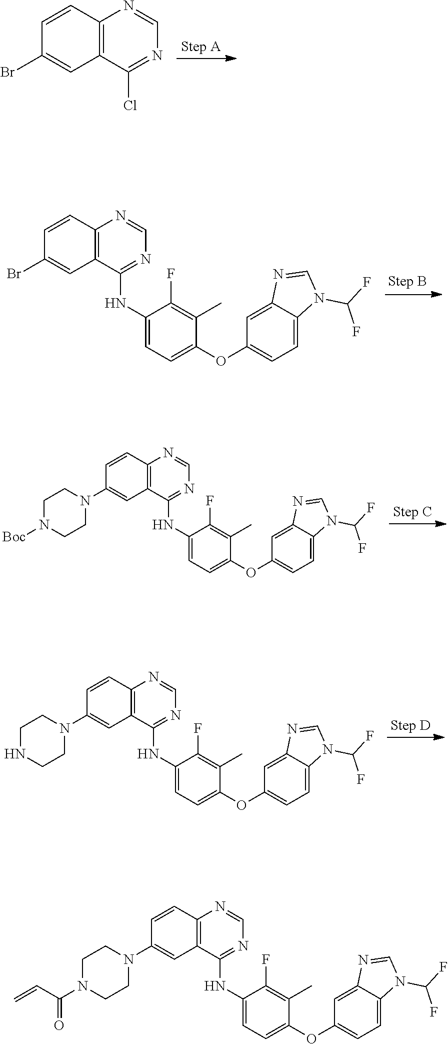 [SMILES: Clc1ncnc2ccc(Br)cc12 ; Cc1c(Oc2ccc3c(c2)ncn3C(F)F)ccc(Nc2ncnc3ccc(Br)cc23)c1F ; Cc1c(Oc2ccc3c(c2)ncn3C(F)F)ccc(Nc2ncnc3ccc(N4CCN(C(=O)OC(C)(C)C)CC4)cc23)c1F ; Cc1c(Oc2ccc3c(c2)ncn3C(F)F)ccc(Nc2ncnc3ccc(N4CCNCC4)cc23)c1F ; C=CC(=O)N1CCN(c2ccc3ncnc(Nc4ccc(Oc5ccc6c(c5)ncn6C(F)F)c(C)c4F)c3c2)CC1]

Step A. 6-bromo-N-(4-((1-(difluoromethyl)-1H-benzo[d]imidazol-5-yl)oxy)-2-fluoro-3-methylphenyl)quinazolin-4-amine. Two batches run in parallel each contained a solution of 4-[1-(difluoromethyl)249midazole249ole-5-yl]oxy-2-fluoro-3-methyl-aniline (400 mg, 1.04 mmol) and 6-bromo-4-chloro-quinazoline (279 mg, 1.2 mmol) in n-BuOH (5 mL). TFA (0.23 mL, 3.1 mmol) was added dropwise, and the mixture was stirred at 90° C. for 2 h. The batches were combined, water (20 mL) was added, and the product was extracted with EtOAc (15 mL×3). The combined organic layers were washed with brine (15 mL×3), dried over anhydrous sodium sulfate, filtered, and concentrated in vacuo. The crude material was purified by silica gel column chromatography (65% to 100% EtOAc/PET) to afford 6-bromo-N-[4-[1-(difluoromethyl)249midazole249ole-5-yl]oxy-2-fluoro-3-methyl-phenyl]quinazolin-4-amine (800 mg, 61% yield) as a brown oil. LCMS (MM-ES+APCI, Pos): m/z 514.1, 516.1 (M+H).

Step B. tert-butyl 4-(4-((4-((1-(difluoromethyl)-1H-benzo[d]250midazole-5-yl)oxy)-2-fluoro-3-methylphenyl)amino)quinazolin-6-yl)piperazine-1-carboxylate. Two batches run in parallel each contained a solution of 6-bromo-N-[4-[1-(difluoromethyl)benzimidazol-5-yl]oxy-2-fluoro-3-methyl-phenyl]quinazolin-4-amine (150 mg, 0.24 mmol) and tert-butyl piperazine-1-carboxylate (267 mg, 1.43 mmol) in 1,4-dioxane (8 mL). Sodium tert-butoxide (0.60 mL, 2 M in THF, 1.20 mmol) and 1,3-bis[2,6-bis(1-ethylpropyl)phenyl]-4,5-dichloro-2H-imidazol-1-ium-2-ide; 3-chloropyridine; dichloropalladium (41 mg, 0.05 mmol) were added, the flask was purged with N₂, and the reaction was stirred at 105° C. for 12 h. The two batches were combined, quenched with water (15 mL) and extracted with EtOAc (10 mL×3). The combined organic layers were washed with brine (20 mL), dried over anhydrous sodium sulfate, filtered, and concentrated under reduced pressure. The crude material was purified by silica gel column chromatography (50% to 100% EtOAc/PET) to afford tert-butyl 4-[4-[4-[1-(difluoromethyl)benzimidazol-5-yl]oxy-2-fluoro-3-methyl-anilino]quinazolin-6-yl]piperazine-1-carboxylate (155 mg, 35% yield) as a yellow oil. LCMS (MM-ES+APCI, Pos): m/z 620.3 (M+H).

Step C. N-(4-((1-(difluoromethyl)-1H-benzo[d]imidazol-5-yl)oxy)-2-fluoro-3-methylphenyl)-6-(piperazin-1-yl)quinazolin-4-amine hydrochloride. To a solution of tert-butyl 4-[4-[4-[1-(difluoromethyl) benzimidazol-5-yl]oxy-2-fluoro-3-methyl-anilino]quinazolin-6-yl]piperazine-1-carboxylate (155 mg, 0.22 mmol) in EtOAc (2 mL) was added HCl (3.6 mL, 2M in EtOAc, 7.2 mmol). The mixture was stirred at 25° C. for 0.5 h and concentrated in vacuo to provide N-[4-[1-(difluoromethyl)benzimidazol-5-yl]oxy-2-fluoro-3-methyl-phenyl]-6-piperazin-1-yl-quinazolin-4-amine hydrochloride (139 mg, 98% yield) as a yellow solid. LCMS (MM-ES+APCI, Pos): m/z 520.3 (M+H—HCl).

Step D. 1-(4-(4-((4-((1-(difluoromethyl)-1H-benzo[d]imidazol-5-yl)oxy)-2-fluoro-3-methylphenyl)amino)quinazolin-6-yl)piperazin-1-yl)prop-2-en-1-one. A solution of N-[4-[1-(difluoromethyl)benzimidazol-5-yl]oxy-2-fluoro-3-methyl-phenyl]-6-piperazin-1-yl-quinazolin-4-amine hydrochloride (130 mg, 0.23 mmol) and DIPEA (81 μL, 0.47 mmol) in DCM (5 mL) was cooled to −78° C. Prop-2-enoyl chloride (19 μL, 0.23 mmol) in DCM (1 mL) was added dropwise and the mixture was stirred for 15 min at −78° C. The reaction was quenched with sat. aq. NaHCO₃ (2 mL) and extracted with EtOAc (10 mL×3). The combined organic layers were dried over anhydrous sodium sulfate, filtered, and concentrated under reduced pressure. The crude product was purified by preparative HPLC (25% MeCN/0.23% aqueous formic acid) and lyophilized to afford 1-[4-[4-[4-[1-(difluoromethyl)benzimidazol-5-yl] oxy-2-fluoro-3-methyl-anilino] quinazolin-6-yl]piperazin-1-yl]prop-2-en-1-one (45 mg, 33% yield) as a yellow solid. LCMS (MM-ES+APCI, Pos): m/z 574.3 (M+H).

### Example 94

### 1-(4-((8-((4-((7-fluoro-1-methyl-1H-benzo[d][1,2,3]triazol-5-yl)oxy)-3-methylphenyl)amino)pyrimido[5,4-d]pyrimidin-2-yl)oxy)piperidin-1-yl)prop-2-en-1-one

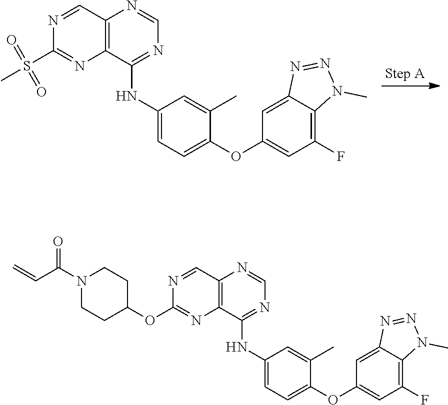

Step A. 1-(4-((8-((4-((7-fluoro-1-methyl-1H-benzo[d][1,2,3]triazol-5-yl)oxy)-3-methylphenyl)amino)pyrimido[5,4-d]pyrimidin-2-yl)oxy)piperidin-1-yl)prop-2-en-1-one. A solution of 1-(4-hydroxy-1-piperidyl)prop-2-en-1-one (43.1 mg, 278 μmol) in THF (3 mL) was cooled to 0° C., followed by addition of NaH (11 mg, 0.28 mmol, 60% wt.). The reaction was stirred at 25° C. for 30 min and N-[4-(7-fluoro-1-methyl-benzotriazol-5-yl)oxy-3-methyl-phenyl]-6-methylsulfonyl-pyrimido[5,4-d]pyrimidin-4-amine (89 mg, 0.19 mmol) was added. After another 30 min the reaction was quenched with sat. aq. NH₄Cl (20 mL) and extracted with EtOAc (30 mL×3). The combined organic layers were washed with brine (30 mL×3), dried over Na₂SO₄, filtered, and concentrated in vacuo. The crude material was purified by preparative HPLC (36% to 66% MeCN/0.1% aqueous FA) to provide 1-[4-[4-[4-(7-fluoro-1-methyl-benzotriazol-5-yl)oxy-3-methyl-anilino]pyrimido[5,4-d]pyrimidin-6-yl]oxy-1-piperidyl]prop-2-en-1-one (35 mg, 32% yield) as a yellow solid. LCMS (MM-ES+APCI, Pos): m/z 556.2 (M+H).

Compounds 106, 376-381 in Table 25 were prepared according to Example 94 step A.

TABLE 25
A list of compounds 106, 376-381

| | | | MS |
|---|---|---|---|
| Cpd. | | | API |
| ID | Structure | Chemical Name | (m/z) |
| 106 | 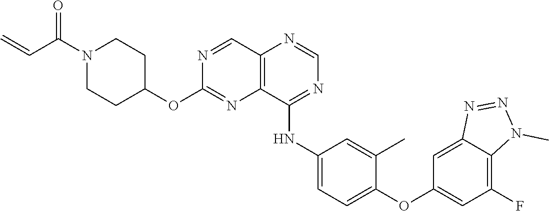 [SMILES: C=CC(=O)N1CCC(Oc2ncc3ncnc(Nc4ccc(Oc5cc(F)c6c(c5)nnn6C)c(C)c4)c3n2)CC1] | 1-(4-((8-((4-((7-fluoro-1-methyl- 1H-benzo[d][1,2,3]triazol-5- yl)oxy)-3- methylphenyl)amino)pyrimido[5,4- d]pyrimidin-2-yl)oxy)piperidin-1- yl)prop-2-en-1-one | 556.2 (M + H) |
| 376 | 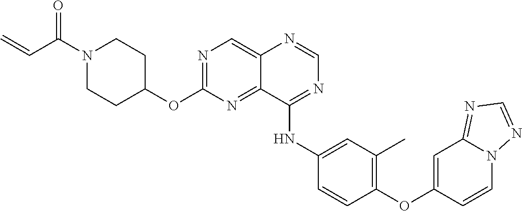 [SMILES: C=CC(=O)N1CCC(Oc2ncc3ncnc(Nc4ccc(Oc5ccn6ncnc6c5)c(C)c4)c3n2)CC1] | 1-(4-((8-((4-([1,2,4]triazolo[1,5- a]pyridin-7-yloxy)-3- methylphenyl)amino)pyrimido[5,4- d]pyrimidin-2-yl)oxy)piperidin-1- yl)prop-2-en-1-one | 524.2 (M + H) |
| 377 | 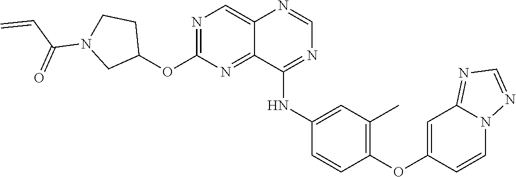 [SMILES: C=CC(=O)N1CCC(Oc2ncc3ncnc(Nc4ccc(Oc5ccn6ncnc6c5)c(C)c4)c3n2)C1] | 1-(3-((8-((4-([1,2,4]triazolo[1,5- a]pyridin-7-yloxy)-3- methylphenyl)amino)pyrimido[5,4- d]pyrimidin-2-yl)oxy)pyrrolidin-1- yl)prop-2-en-1-one | 510.2 (M + H) |
| 378 | 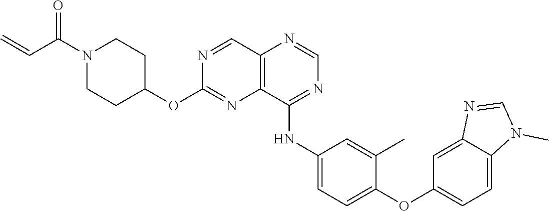 [SMILES: C=CC(=O)N1CCC(Oc2ncc3ncnc(Nc4ccc(Oc5ccc6c(c5)ncn6C)c(C)c4)c3n2)CC1] | 1-(4-((8-((3-methyl-4-((1-methyl- 1H-benzo[d]imidazol-5- yl)oxy)phenyl)amino)pyrimido[5,4- d]pyrimidin-2-yl)oxy)piperidin-1- yl)prop-2-en-1-one | 537.3 (M + H) |
| 379 | 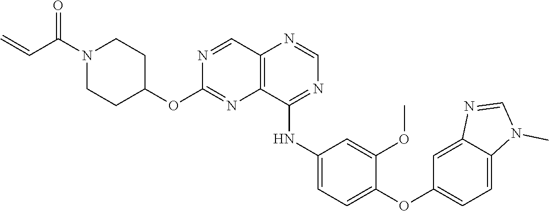 [SMILES: C=CC(=O)N1CCC(Oc2ncc3ncnc(Nc4ccc(Oc5ccc6c(c5)ncn6C)c(OC)c4)c3n2)CC1] | 1-(4-((8-((3-methoxy-4-((1-methyl- 1H-benzo[d]imidazol-5- yl)oxy)phenyl)amino)pyrimido[5,4- d]pyrimidin-2-yl)oxy)piperidin-1- yl)prop-2-en-1-one | 553.3 (M + H) |
| 380 | 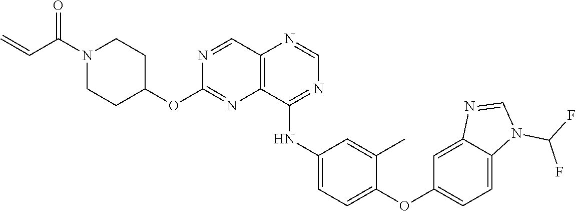 [SMILES: C=CC(=O)N1CCC(Oc2ncc3ncnc(Nc4ccc(Oc5ccc6c(c5)ncn6C(F)F)c(C)c4)c3n2)CC1] | 1-(4-((8-((4-((1-(difluoromethyl)- 1H-benzo[d]imidazol-5-yl)oxy)-3- methylphenyl)amino)pyrimido[5,4- d]pyrimidin-2-y1)oxy)piperidin-1- yl)prop-2-en-1-one | 573.3 (M + H) |
| 381 | 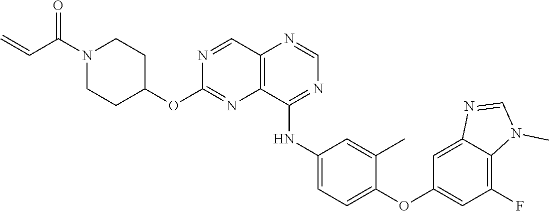 [SMILES: C=CC(=O)N1CCC(Oc2ncc3ncnc(Nc4ccc(Oc5cc(F)c6c(c5)ncn6C)c(C)c4)c3n2)CC1] | 1-(4-((8-((4-((7-fluoro-1-methyl- 1H-benzo[d]imidazol-5-yl)oxy)-3- methylphenyl)amino)pyrimido[5,4- d]pyrimidin-2-yl)oxy)piperidin-1- yl)prop-2-en-1-one | 555.3 (M + H) |

### Example 95

### N-methyl-N-((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)acrylamide (compound 382)

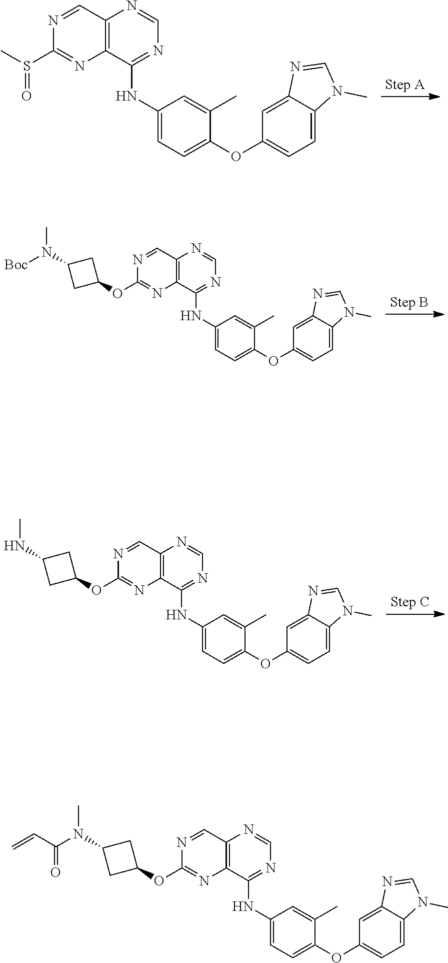

Step A. tert-butyl methyl((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)carbamate. To a solution of tert-butyl ((1R,3R)-3-hydroxycyclobutyl)(methyl)carbamate (108 mg, 0.54 mmol) in THF (5 mL) at 0° C. was added NaH (35.9 mg, 0.90 mmol, 60% w/w). The mixture was stirred 30 min and N-(3-methyl-4-((1-methyl-1H-benzo[d]imidazol-5-yl)oxy)phenyl)-6-(methylsulfinyl)pyrimido[5,4-d]pyrimidin-4-amine (200 mg, 0.45 mmol) was added at 0° C. The mixture was warmed to 25° C. for 1 h. The reaction was quenched with sat. aq. NH₄Cl (30 mL) and extracted with DCM (30 mL×2). The combined organic phases were washed with water (20 mL×3), dried over anhydrous Na₂SO₄, filtered and concentrated under reduced pressure. The crude residue was purified by silica gel column chromatography (0% to 10% MeOH/DCM) to afford tert-butyl methyl((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)carbamate (200 mg, 66% yield) as a yellow solid. LCMS (MM-ES+APCI, Pos): m/z 583.4 (M+H).

Step B. N-(3-methyl-4-((1-methyl-1H-benzo[d]imidazol-5-yl)oxy)phenyl)-6-((1R,3R)-3-(methylamino)cyclobutoxy)pyrimido[5,4-d]pyrimidin-4-amine. A mixture of tert-butyl methyl((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)carbamate (175 mg, 0.30 mmol) and TFA (1 mL, 13.5 mmol) in DCM (4 mL) was stirred at 25° C. for 10 min. The mixture was concentrated under reduced pressure to give N-(3-methyl-4-((1-methyl-1H-benzol[d]imidazol-5-yl)oxy)phenyl)-6-((1R,3R)-3-(methylamino)cyclobutoxy)pyrimido[5,4-d]pyrimidin-4-amine trifluoroacetate (179 mg, quant. yield) as a yellow oil. LCMS (MM-ES+APCI, Pos): m/z 483.1 (M+H-TFA).

Step C. N-methyl-N-((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)acrylamide. To a solution of N-(3-methyl-4-((1-methyl-1H-benzo[d]imidazol-5-yl)oxy)phenyl)-6-((1R,3R)-3-(methylamino)cyclobutoxy)pyrimido[5,4-d]pyrimidin-4-amine trifluoroacetate (170 mg, 0.29 mmol) in DCM (4 mL) was added TEA (0.20 mL, 1.42 mmol). The mixture was cooled to −40° C. and acryloyl chloride (35 uL, 0.43 mmol) in DCM (2 mL) was added dropwise. After stirring for 30 min the mixture was concentrated under reduced pressure. The crude residue was purified by preparative HPLC (25% to 55% MeCN/0.1% NH₄HCO₃) and lyophilized to afford N-methyl-N-((1R,3R)-3-((8-((3-methyl-4-((1-methyl-1H-benzo[d]imidazol-5-yl)oxy)phenyl)amino)pyrimido[5,4-d]pyrimidin-2-yl)oxy)cyclobutyl)acrylamide (46.2 mg, 30% yield) as a yellow solid. LCMS (MM-ES+APCI, Pos): m/z 537.3 (M+H).

Compounds 382 to 393 in Table 26 were prepared according to Example 95 steps A to C.

TABLE 26
A list of compounds 382-393

| | | | MS |
|---|---|---|---|
| Cpd. | | | API |
| ID | Structure | Chemical Name | (m/z) |
| 382 | 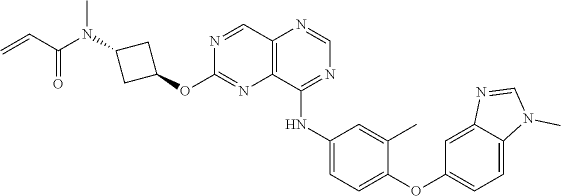 [SMILES: C=CC(=O)N(C)[C@H]1C[C@H](Oc2ncc3ncnc(Nc4ccc(Oc5ccc6c(c5)ncn6C)c(C)c4)c3n2)C1] | N-methyl-N-((1r,3r)-3-((8-((3- methyl-4-((1-methyl-1H- benzo[d]imidazol-5- yl)oxy)phenyl)amino)pyrimido[5,4- d]pyrimidin-2- yl)oxy)cyclobutyl)acrylamide | 537.3 (M + H) |
| 383 | 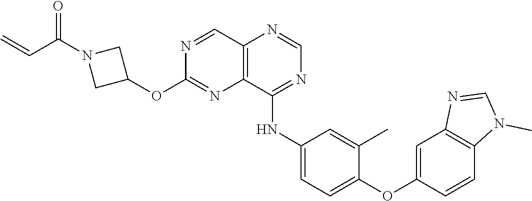 [SMILES: C=CC(=O)N1CC(Oc2ncc3ncnc(Nc4ccc(Oc5ccc6c(c5)ncn6C)c(C)c4)c3n2)C1] | 1-(3-((8-((3-methyl-4-((1-methyl- 1H-benzo[d]imidazol-5- yl)oxy)phenyl)amino)pyrimido[5,4- d]pyrimidin-2-yl)oxy)azetidin-1- yl)prop-2-en-1-one | 509.1 (M + H) |
| 384 | 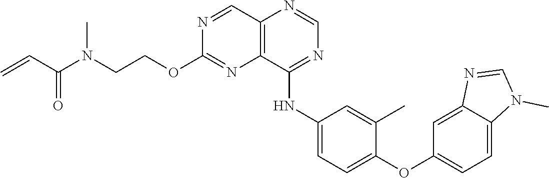 [SMILES: C=CC(=O)N(C)CCOc1ncc2ncnc(Nc3ccc(Oc4ccc5c(c4)ncn5C)c(C)c3)c2n1] | N-methyl-N-(2-((8-((3-methyl-4- ((1-methyl-1H-benzo[d]imidazol-5- yl)oxy)phenyl)amino)pyrimido[5,4- d]pyrimidin-2- yl)oxy)ethyl)acrylamide | 511.1 (M + H) |
| 385 | 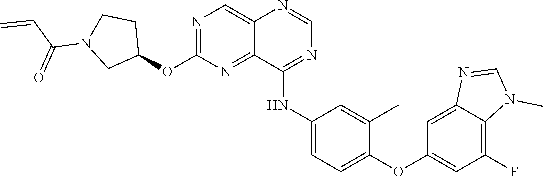 [SMILES: C=CC(=O)N1CC[C@@H](Oc2ncc3ncnc(Nc4ccc(Oc5cc(F)c6c(c5)ncn6C)c(C)c4)c3n2)C1] | (R)-1-(3-((8-((4-((7-fluoro-1- methyl-1H-benzo[d]imidazol-5- yl)oxy)-3- methylphenyl)amino)pyrimido[5,4- d]pyrimidin-2-yl)oxy)pyrrolidin-1- yl)prop-2-en-1-one | 541.3 (M + H) |
| 386 | 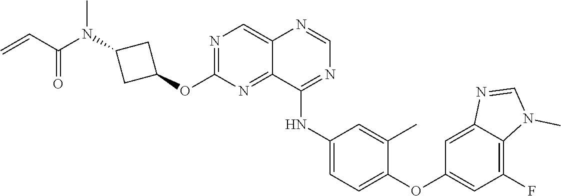 [SMILES: C=CC(=O)N(C)[C@H]1C[C@H](Oc2ncc3ncnc(Nc4ccc(Oc5cc(F)c6c(c5)ncn6C)c(C)c4)c3n2)C1] | N-((1r,3r)-3-((8-((4-((7-fluoro-1- methyl-1H-benzo[d]imidazol-5- yl)oxy)-3- methylphenyl)amino)pyrimido[5,4- d]pyrimidin-2-yl)oxy)cyclobutyl)- N-methylacrylamide | 555.3 (M + H) |
| 387 | 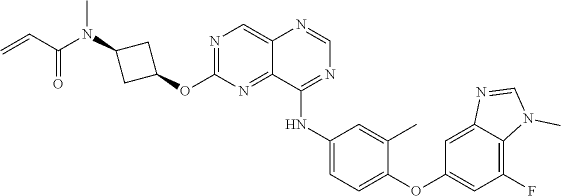 [SMILES: C=CC(=O)N(C)[C@H]1C[C@@H](Oc2ncc3ncnc(Nc4ccc(Oc5cc(F)c6c(c5)ncn6C)c(C)c4)c3n2)C1] | N-((1s,3s)-3-((8-((4-((7-fluoro-1- methyl-1H-benzo[d]imidazol-5- yl)oxy)-3- methylphenyl)amino)pyrimido[5,4- d]pyrimidin-2-yl)oxy)cyclobutyl)- N-methylacrylamide | 555.1 (M + H) |
| 388 | 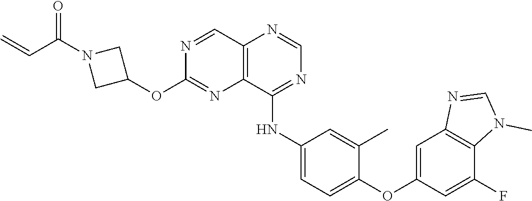 [SMILES: C=CC(=O)N1CC(Oc2ncc3ncnc(Nc4ccc(Oc5cc(F)c6c(c5)ncn6C)c(C)c4)c3n2)C1] | 1-(3-((8-((4-((7-fluoro-1-methyl- 1H-benzo[d]imidazol-5-yl)oxy)-3- methylphenyl)amino)pyrimido[5,4- d]pyrimidin-2-yl)oxy)azetidin-1- yl)prop-2-en-1-one | 527.3 (M + H) |
| 389 | 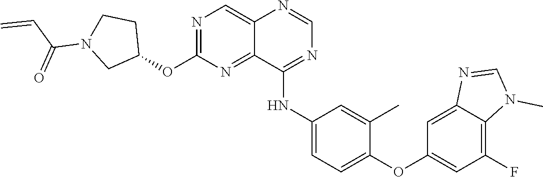 [SMILES: C=CC(=O)N1CC[C@H](Oc2ncc3ncnc(Nc4ccc(Oc5cc(F)c6c(c5)ncn6C)c(C)c4)c3n2)C1] | (S)-1-(3-((8-((4-((7-fluoro-1- methyl-1H-benzo[d]imidazol-5- yl)oxy)-3- methylphenyl)amino)pyrimido[5,4- d]pyrimidin-2-yl)oxy)pyrrolidin-1- yl)prop-2-en-1-one | 541.2 (M + H) |
| 390 | 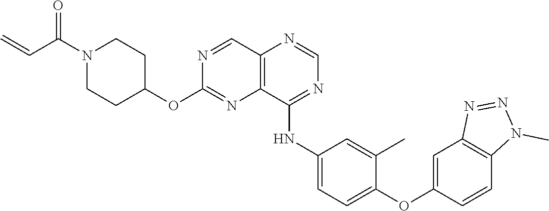 [SMILES: C=CC(=O)N1CCC(Oc2ncc3ncnc(Nc4ccc(Oc5ccc6c(c5)nnn6C)c(C)c4)c3n2)CC1] | 1-(4-((8-((3-methyl-4-((1-methyl- 1H-benzo[d][1,2,3]triazol-5- yl)oxy)phenyl)amino)pyrimido[5,4- d]pyrimidin-2-yl)oxy)piperidin-1- yl)prop-2-en-1-one | 538.3 (M + H) |
| 391 | 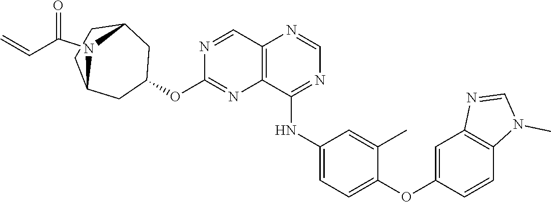 [SMILES: C=CC(=O)N1[C@@H]2CC[C@H]1C[C@@H](Oc1ncc3ncnc(Nc4ccc(Oc5ccc6c(c5)ncn6C)c(C)c4)c3n1)C2] | 1-((1R,3r,5S)-3-((8-((3-methyl-4- ((1-methyl-1H-benzo[d]imidazol-5- yl)oxy)phenyl)amino)pyrimido[5,4- d]pyrimidin-2-yl)oxy)-8- azabicyclo[3.2.1]octan-8-yl)prop-2- en-1-one | 563.3 (M + H) |
| 392 | 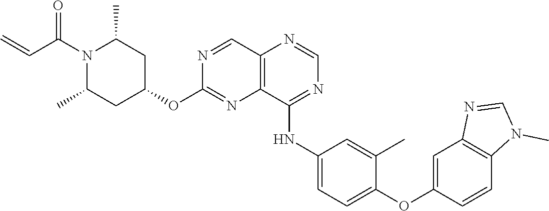 [SMILES: C=CC(=O)N1[C@H](C)C[C@H](Oc2ncc3ncnc(Nc4ccc(Oc5ccc6c(c5)ncn6C)c(C)c4)c3n2)C[C@@H]1C] | 1-((2R,4r,6S)-2,6-dimethyl-4-((8- ((3-methyl-4-((1-methyl-1H- benzo[d]imidazol-5- yl)oxy)phenyl)amino)pyrimido[5,4- d]pyrimidin-2-yl)oxy)piperidin-1- yl)prop-2-en-1-one | 565.3 (M + H) |
| 393 | 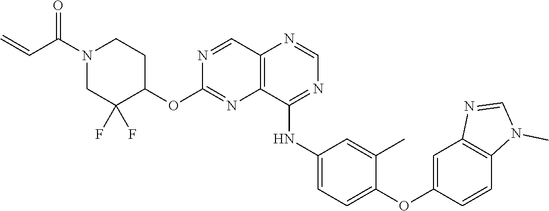 [SMILES: C=CC(=O)N1CCC(Oc2ncc3ncnc(Nc4ccc(Oc5ccc6c(c5)ncn6C)c(C)c4)c3n2)C(F)(F)C1] | 1-(3,3-difluoro-4-((8-((3-methyl-4- ((1-methyl-1H-benzo[d]imidazol-5- yl)oxy)phenyl)amino)pyrimido[5,4- d]pyrimidin-2-y1)oxy)piperidin-1- yl)prop-2-en-1-one | 573.2 (M + H) |

### Example 96

### 1-(4-((4-((2-fluoro-3-methyl-4-((1-methyl-1H-benzo[1d]imidazol-5-yl)oxy)phenyl)amino)-7-methoxypyrido[3,2-d]pyrimidin-6-yl)oxy)piperidin-1-yl)prop-2-en-1-one (compound 394)

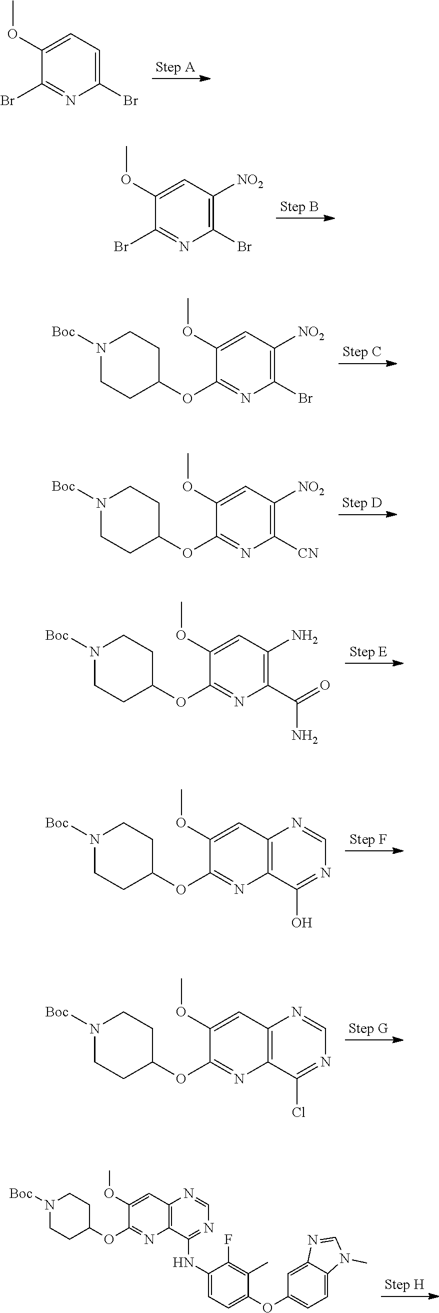

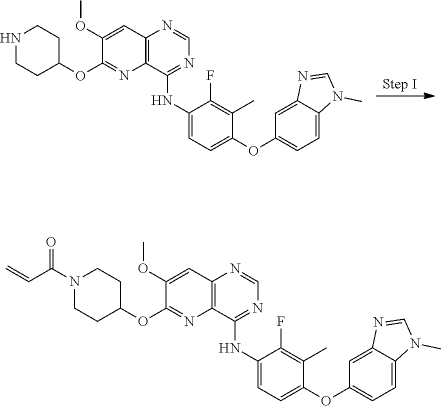

Step A. 2,6-dibromo-3-methoxy-5-nitropyridine. A solution of HNO₃ (10 mL) and H₂SO₄ (10 mL) was cooled to 0° C. and 2,6-dibromo-3-methoxy-pyridine (2.00 g, 7.49 mmol) was added in portions. The reaction was heated to 60° C. for 2 h. The mixture was cooled to 25° C. and slowly poured into ice water at 0-5° C. The aqueous mixture was extracted with EtOAc (50 mL×3), and the combined organic layers were washed with brine (50 mL), dried over anhydrous sodium sulfate, filtered and concentrated under reduced pressure. The crude residue was purified by silica gel column chromatography (10% to 20% EtOAc/PET) to afford 2,6-dibromo-3-methoxy-5-nitro-pyridine (1.1 g, 47% yield) as a white solid.

Step B. tert-butyl 4-((6-bromo-3-methoxy-5-nitropyridin-2-yl)oxy)piperidine-1-carboxylate. A solution of tert-butyl 4-hydroxypiperidine-1-carboxylate (637 mg, 3.16 mmol) in THF (10 mL) was placed under N₂ and cooled to 0° C. NaH (253 mg, 6.33 mmol, 60% w/w) was added and the reaction was stirred for 30 min before 2,6-dibromo-3-methoxy-5-nitro-pyridine 2 (1.00 g, 3.16 mmol) in THF (5 mL) was added dropwise at 0° C. The resulting mixture was allowed to warm to 25° C. and stir for 2 h. The reaction was quenched with sat. aq. NH₄Cl and extracted with EtOAc (50 mL×3). The combined organic layers were washed with brine (50 mL), dried over anhydrous sodium sulfate, filtered and concentrated under reduced pressure. The crude residue was purified by silica gel column chromatography (10% to 20% EtOAc/PET) to afford tert-butyl 4-[(6-bromo-3-methoxy-5-nitro-2-pyridyl)oxy]piperidine-1-carboxylate (1.05 g, 76% yield) as a white solid. LCMS (MM-ES+APCI, Pos): m/z 376.0, 378.0 (M+H-^{t}Bu).

Step C. tert-butyl 4-((6-cyano-3-methoxy-5-nitropyridin-2-yl)oxy)piperidine-1-carboxylate. To a solution of tert-butyl 4-[(6-bromo-3-methoxy-5-nitro-2-pyridyl)oxy]piperidine-1-carboxylate (1.0 g, 2.31 mmol) in DMF (10 mL) was added CuCN (0.41 g, 4.61 mmol). The mixture was stirred at 100° C. for 4 h. The reaction was quenched with water (30 mL) and extracted with EtOAc (30 mL×3). The combined organic layers were washed with brine (60 mL), dried over anhydrous sodium sulfate, filtered, and concentrated under reduced pressure to afford tert-butyl 4-[(6-cyano-3-methoxy-5-nitro-2-pyridyl)oxy]piperidine-1-carboxylate 5 (1.0 g, quant. yield) as a gray solid. LCMS (MM-ES+APCI, Pos): m/z 323.1 (M+H-^{t}Bu).

Step D. tert-butyl 4-((5-amino-6-carbamoyl-3-methoxypyridin-2-yl)oxy)piperidine-1-carboxylate. To a solution of tert-butyl 4-[(6-cyano-3-methoxy-5-nitro-2-pyridyl)oxy]piperidine-1-carboxylate (950 mg, 2.51 mmol) in EtOH (10 mL) and water (1 mL) was added Fe powder (561 mg, 10.0 mmol) and NH₄Cl (537 mg, 10.0 mmol). The mixture was stirred at 80° C. for 4 h. The suspension was filtered and the filter cake was washed with MeOH (20 mL). The filtrate was concentrated under reduced pressure affording tert-butyl 4-[(5-amino-6-carbamoyl-3-methoxy-2-pyridyl)oxy]piperidine-1-carboxylate (860 mg, 93% yield) as a gray solid. LCMS (MM-ES+APCI, Pos): m/z 367.2 (M+H).

Step E. tert-butyl 4-((4-hydroxy-7-methoxypyrido[3,2-d]pyrimidin-6-yl)oxy)piperidine-1-carboxylate. A mixture of tert-butyl 4-[(5-amino-6-carbamoyl-3-methoxy-2-pyridyl)oxy]piperidine-1-carboxylate (400 mg, 1.09 mmol) and CH(OEt)₃ (10 mL) was heated to 100° C. and stirred for 4 h. The mixture was concentrated under reduced pressure, and the crude residue was purified by silica gel column chromatography (5% to 10% MeOH/DCM) to give tert-butyl 4-(4-hydroxy-7-methoxy-pyrido[3,2-d]pyrimidin-6-yl)oxypiperidine-1-carboxylate (200 mg, 48% yield) as a white solid. LCMS (MM-ES+APCI, Pos): m/z 377.0 (M+H).

Steps F to G. tert-butyl 4-((4-((2-fluoro-3-methyl-4-((1-methyl-1H-benzo[d]imidazol-5-yl)oxy)phenyl)amino)-7-methoxypyrido[3,2-d]pyrimidin-6-yl)oxy)piperidine-1-carboxylate. Prepared according to Example 88 steps E to F, substituting tert-butyl 4-((4-hydroxy-7-methoxypyrido[3,2-d]pyrimidin-6-yl)oxy)piperidine-1-carboxylate for 6-bromo-7-fluoropyrido[3,2-d]pyrimidin-4-ol to afford tert-butyl 4-((4-((2-fluoro-3-methyl-4-((1-methyl-1H-benzo[d]imidazol-5-yl)oxy)phenyl)amino)-7-methoxypyrido[3,2-d]pyrimidin-6-yl)oxy)piperidine-1-carboxylate (110 mg, 69% yield) as a brown oil. LCMS (MM-ES+APCI, Pos): m/z 530.3 (M+H-Boc).

Steps H to I. 1-(4-((4-((2-fluoro-3-methyl-4-((1-methyl-1H-benzo[d]imidazol-5-yl)oxy)phenyl)amino)-7-methoxypyrido[3,2-d]pyrimidin-6-yl)oxy)piperidin-1-yl)prop-2-en-1-one. Prepared according to Example 88 steps H to I, substituting tert-butyl 4-((4-((2-fluoro-3-methyl-4-((1-methyl-1H-benzo[d]imidazol-5-yl)oxy)phenyl)amino)-7-methoxypyrido[3,2-d]pyrimidin-6-yl)oxy)piperidine-1-carboxylate for tert-butyl 4-(7-fluoro-4-((3-methyl-4-((1-methyl-1H-benzo[d][1,2,3]triazol-5-yl)oxy)phenyl)amino)pyrido[3,2-d]pyrimidin-6-yl)piperazine-1-carboxylate to afford 1-(4-((4-((2-fluoro-3-methyl-4-((1-methyl-1H-benzo[d]imidazol-5-yl)oxy)phenyl)amino)-7-methoxypyrido[3,2-d]pyrimidin-6-yl)oxy)piperidin-1-yl)prop-2-en-1-one (15 mg, 20% yield) as a white solid. LCMS (MM-ES+APCI, Pos): m/z 584.2 (M+H).

### Example 97

### 1-(4-((8-((2-fluoro-3-methyl-4-((1-methyl-1H-benzo[d]imidazol-5-yl)oxy)phenyl)amino)pyrimido[5,4-d]pyrimidin-2-yl)thio)piperidin-1-yl)prop-2-en-1-one (compound 107)

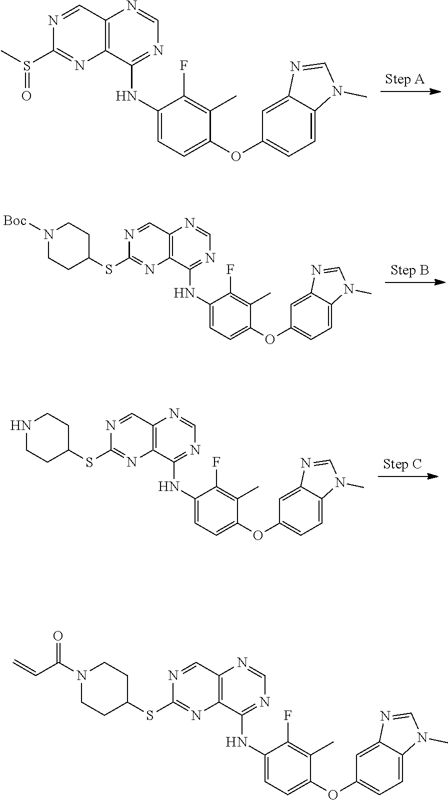

Step A. tert-butyl 4-((8-((2-fluoro-3-methyl-4-((1-methyl-1H-benzo[d]imidazol-5-yl)oxy)phenyl)amino)pyrimido[5,4-d]pyrimidin-2-yl)thio)piperidine-1-carboxylate. To a solution of N-[2-fluoro-3-methyl-4-(1-methylbenzimidazol-5-yl)oxy-phenyl]-6-methylsulfinyl-pyrimido[5,4-d]pyrimidin-4-amine (150 mg, 0.32 mmol) in THF (2 mL) were added tert-butyl 4-sulfanylpiperidine-1-carboxylate (90 mg, 0.41 mmol) and Cs₂CO₃ (320 mg, 0.98 mmol). The mixture was stirred at 60° C. for 2 h and quenched with H₂O (20 mL). The layers were partitioned, and the aqueous layer was extracted with DCM (20 mL×3). The combined organic layers were washed with brine (20 mL), dried over anhydrous Na₂SO₄, filtered, and concentrated under reduced pressure to afford tert-butyl 4-[4-[2-fluoro-3-methyl-4-(1-methylbenzimidazol-5-yl)oxy-anilino]pyrimido[5,4-d]pyrimidin-6-yl]sulfanylpiperidine-1-carboxylate (260 mg, quant. yield) as a yellow solid. LCMS (MM-ES+APCI, Pos): m/z 617.2 (M+H).

Step B. N-(2-fluoro-3-methyl-4-((1-methyl-1H-benzo[d]imidazol-5-yl)oxy)phenyl)-6-(piperidin-4-ylthio)pyrimido[5,4-d]pyrimidin-4-amine hydrochloride. To a solution of tert-butyl 4-[4-[2-fluoro-3-methyl-4-(1-methylbenzimidazol-5-yl)oxy-anilino]pyrimido[5,4-d]pyrimidin-6-yl]sulfanylpiperidine-1-carboxylate (160 mg, 0.26 mmol) in DCM (2 mL) was added HCl (4 M in 1,4-dioxane, 0.62 mL, 2.48 mmol). The mixture was stirred at 25° C. for 2 h and concentrated in vacuo to afford N-[2-fluoro-3-methyl-4-(1-methylbenzimidazol-5-yl)oxy-phenyl]-6-(4-piperidylsulfanyl)pyrimido[5,4-d]pyrimidin-4-amine hydrochloride (120 mg, 83% yield) as a yellow solid. LCMS (MM-ES+APCI, Pos): m/z 517.2 (M+H—HCl).

Step C. 1-(4-((8-((2-fluoro-3-methyl-4-((1-methyl-1H-benzo[d]imidazol-5-yl)oxy)phenyl)amino)pyrimido[5,4-d]pyrimidin-2-yl)thio)piperidin-1-yl)prop-2-en-1-one. To a solution of N-[2-fluoro-3-methyl-4-(1-methylbenzimidazol-5-yl)oxy-phenyl]-6-(4-piperidylsulfanyl)pyrimido[5,4-d]pyrimidin-4-amine hydrochloride (100 mg, 0.18 mmol) in DCM (1.5 mL) was added TEA (76 μL, 0.54 mmol). The solution was cooled to −40° C. and prop-2-enoyl chloride (15 μL, 0.18 mmol) was added. The mixture was stirred at −40° C. for 1 h and concentrated under reduced pressure. The crude material was purified by preparative HPLC (31% to 61% MeCN/0.08% aqueous NH₄HCO₃) to provide 1-[4-[4-[2-fluoro-3-methyl-4-(1-methylbenzimidazol-5-yl)oxy-anilino]pyrimido[5,4-d]pyrimidin-6-yl]sulfanyl-1-piperidyl]prop-2-en-1-one (25 mg, 24% yield) as a yellow solid. LCMS (MM-ES+APCI, Pos): m/z 571.1 (M+H).

### Example 98

### 1-(4-(4-((3-methyl-4-((1-methyl-1H-benzo[d][1,2,3]triazol-5-yl)oxy)phenyl)amino)pyrido[3,2-d]pyrimidin-6-yl)piperidin-1-yl)prop-2-en-1-one (compound 108)

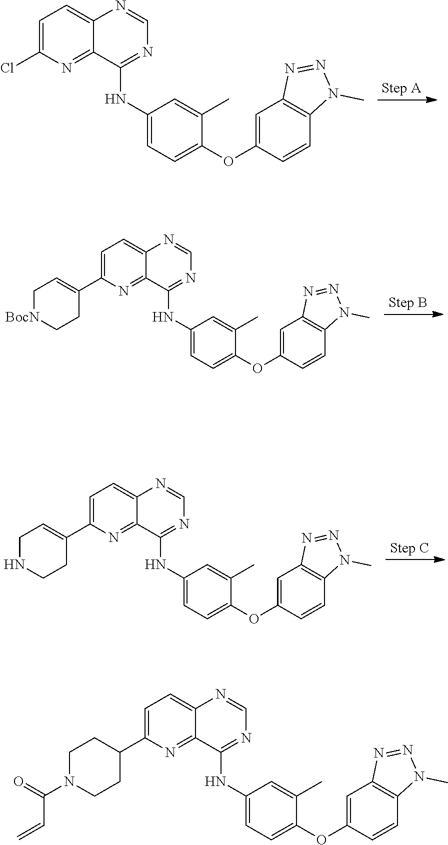 [SMILES: Cc1cc(Nc2ncnc3ccc(Cl)nc23)ccc1Oc1ccc2c(c1)nnn2C ; Cc1cc(Nc2ncnc3ccc(C4=CCN(C(=O)OC(C)(C)C)CC4)nc23)ccc1Oc1ccc2c(c1)nnn2C ; Cc1cc(Nc2ncnc3ccc(C4=CCNCC4)nc23)ccc1Oc1ccc2c(c1)nnn2C ; C=CC(=O)N1CCC(c2ccc3ncnc(Nc4ccc(Oc5ccc6c(c5)nnn6C)c(C)c4)c3n2)CC1]

Step A. tert-butyl 4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridine-1(2H)-carboxylate. To a slurry of 6-chloro-N-(3-methyl-4-((1-methyl-H-benzo[d][1,2,3]triazol-5-yl)oxy)phenyl)pyrido[3,2-d]pyrimidin-4-amine (100 mg, 0.24 mmol), K₂CO₃ (132 mg, 0.96 mmol), and tert-butyl 4-(4,4,5,5-tetramethyl-1,3,2-dioxaborolan-2-yl)-3,6-dihydropyridine-1(2H)-carboxylate (81.4 mg, 0.26 mmol) in 1,4-dioxane (1.00 mL) and water (0.25 mL) was added Pd(PPh₃)₄ (27.7 mg, 23.9 μmol). The reaction vessel was sealed and heated to 100° C. for 3 h. The mixture was filtered over celite and concentrated in vacuo. The crude reaction was purified by silica gel chromatography (0% to 20% MeOH/DCM) to afford tert-butyl 4-(4-((3-methyl-4-((1-methyl-H-benzo[d][1,2,3]triazol-5-yl)oxy)phenyl)amino)pyrido[3,2-d]pyrimidin-6-yl)-3,6-dihydropyridine-1(2H)-carboxylate (140.4 mg, 88% yield). LCMS (MM-ES+APCI, Pos): m/z 565.3 (M+H).

Step B. N-(3-methyl-4-((1-methyl-1H-benzo[d][1,2,3]triazol-5-yl)oxy)phenyl)-6-(piperidin-4-yl)pyrido[3,2-d]pyrimidin-4-amine. To a slurry of tert-butyl 4-(4-((3-methyl-4-((1-methyl-1H-benzo[d]1[1,2,3]triazol-5-yl)oxy)phenyl)amino)pyrido[3,2-d]pyrimidin-6-yl)-3,6-dihydropyridine-1(2H)-carboxylate (85.0 mg, 0.15 mmol) and Pd/Al₂O₃ (5% wt., 25.6 mg, 2.0 μmol) in MeOH (2.00 mL) was added ammonium formate (47.5 mg, 0.75 mmol). The reaction was heated to reflux for 2 h, filtered over celite, and concentrated in vacuo. The crude residue was dissolved in DCM (1.00 mL) and TFA (10 μL, 0.15 mmol). The reaction was stirred for 1 h, concentrated in vacuo, and purified by preparative HPLC (0% to 100% [0.1% TFA in MeCN]/0.1% aqueous TFA). The product was neutralized with sat. aq. NaHCO₃ and extracted with 20% MeOH/DCM to afford N-(3-methyl-4-((1-methyl-1H-benzo[d]1[1,2,3]triazol-5-yl)oxy)phenyl)-6-(piperidin-4-yl)pyrido[3,2-d]pyrimidin-4-amine (47.5 mg, 68% yield). LCMS (MM-ES+APCI, Pos): m/z 467.3 (M+H).

Step C. 1-(4-(4-((3-methyl-4-((1-methyl-H-benzo[d][1,2,3]triazol-5-yl)oxy)phenyl)amino)pyrido[3,2-d]pyrimidin-6-yl)piperidin-1-yl)prop-2-en-1-one. A slurry of N-(3-methyl-4-((1-methyl-H-benzo[d][1,2,3]triazol-5-yl)oxy)phenyl)-6-(piperidin-4-yl)pyrido[3,2-d]pyrimidin-4-amine (48 mg, 0.10 mmol) in THF (1 mL) was cooled to −78° C., after which TEA (28 μL, 0.20 mmol) was added. The solution was stirred at −78° C. for 5 min and acryloyl chloride (9.9 μL, 0.12 mmol) was added. After 15 min at −78° C., the reaction was removed from the dry ice bath and stirred an additional 15 min before being quenched with sat. aq. NaHCO₃ (5 drops). The reaction was warmed to ambient temperature and concentrated in vacuo. The crude product was purified by silica gel chromatography (0% to 20% MeOH/DCM) to afford 1-(4-(4-((3-methyl-4-((1-methyl-1H-benzo[d]1[1,2,3]triazol-5-yl)oxy)phenyl)amino)pyrido[3,2-d]pyrimidin-6-yl)piperidin-1-yl)prop-2-en-1-one (25 mg, 45% yield) as a white solid. LCMS (MM-ES+APCI, Pos): m/z 521.3 (M+H).

Compounds 108, 395-402 in Table 27 were prepared according to Example 98 steps A to C.

TABLE 27
A list of compounds 108, 395-402

| | | | MS |
|---|---|---|---|
| Cpd. | | | API |
| ID | Structure | Chemical Name | (m/z) |
| 108 | 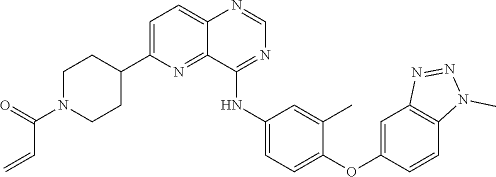 [SMILES: C=CC(=O)N1CCC(c2ccc3ncnc(Nc4ccc(Oc5ccc6c(c5)nnn6C)c(C)c4)c3n2)CC1] | 1-(4-(4-((3-methyl-4-((1-methyl- 1H-benzo[d][1,2,3]triazol-5- yl)oxy)phenyl)amino)pyrido[3,2- d]pyrimidin-6-yl)piperidin-1- yl)prop-2-en-1-one | 521.3 (M + H) |
| 395 | 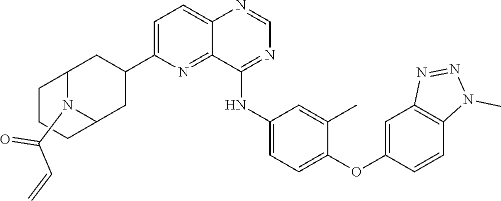 [SMILES: C=CC(=O)N1C2CCCC1CC(c1ccc3ncnc(Nc4ccc(Oc5ccc6c(c5)nnn6C)c(C)c4)c3n1)C2] | 1-(3-(4-((3-methyl-4-((1-methyl- 1H-benzo[d][1,2,3]triazol-5- yl)oxy)phenyl)amino)pyrido[3,2- d]pyrimidin-6-yl)-9- azabicyclo[3.3.1]nonan-9- yl)prop-2-en-1-one trifluoroacetate | 561.3 (M + H − TFA) |
| 396 | 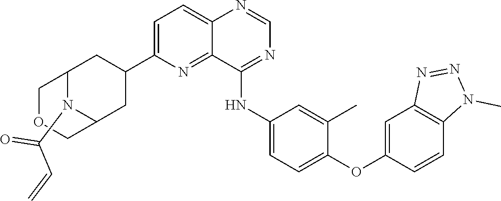 [SMILES: C=CC(=O)N1C2COCC1CC(c1ccc3ncnc(Nc4ccc(Oc5ccc6c(c5)nnn6C)c(C)c4)c3n1)C2] | 1-(7-(4-((3-methyl-4-((1-methyl- 1H-benzo[d][1,2,3]triazol-5- yl)oxy)phenyl)amino)pyrido[3,2- d]pyrimidin-6-y1)-3-oxa-9- azabicyclo[3.3.1]nonan-9- yl)prop-2-en-1-one trifluoroacetate | 563.3 (M + H − TFA) |
| 397 | 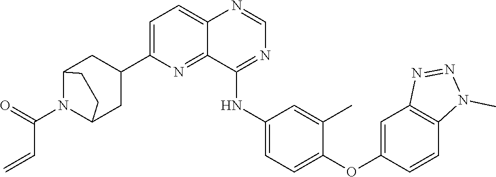 [SMILES: C=CC(=O)N1C2CCC1CC(c1ccc3ncnc(Nc4ccc(Oc5ccc6c(c5)nnn6C)c(C)c4)c3n1)C2] | 1-(3-(4-((3-methyl-4-((1-methyl- 1H-benzo[d][1,2,3]triazol-5- yl)oxy)phenyl)amino)pyrido[3,2- d]pyrimidin-6-y1)-8- azabicyclo[3.2.1]octan-8- yl)prop-2-en-1-one trifluoroacetate | 547.3 (M + H − TFA) |
| 398 | 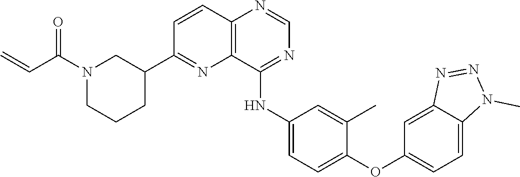 [SMILES: C=CC(=O)N1CCCC(c2ccc3ncnc(Nc4ccc(Oc5ccc6c(c5)nnn6C)c(C)c4)c3n2)C1] | 1-(3-(4-((3-methyl-4-((1-methyl- 1H-benzo[d][1,2,3]triazol-5- yl)oxy)phenyl)amino)pyrido[3,2- d]pyrimidin-6-yl)piperidin-1- yl)prop-2-en-1-one | 521.3 (M + H) |
| 399 | 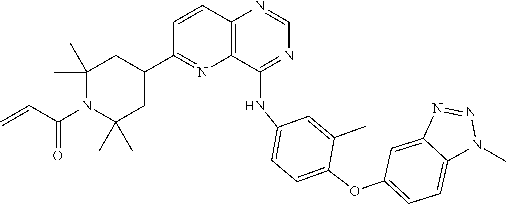 [SMILES: C=CC(=O)N1C(C)(C)CC(c2ccc3ncnc(Nc4ccc(Oc5ccc6c(c5)nnn6C)c(C)c4)c3n2)CC1(C)C] | 1-(2,2,6,6-tetramethyl-4-(4-((3- methyl-4-((1-methyl-1H- benzo[d][1,2,3]triazol-5- yl)oxy)phenyl)amino)pyrido[3,2- d]pyrimidin-6-yl)piperidin-1- yl)prop-2-en-1-one | 577.3 (M + H) |
| 400 | 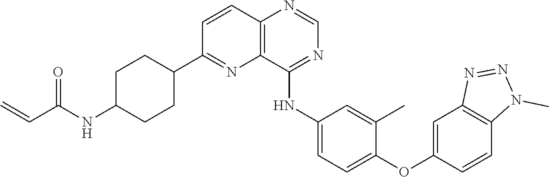 [SMILES: C=CC(=O)NC1CCC(c2ccc3ncnc(Nc4ccc(Oc5ccc6c(c5)nnn6C)c(C)c4)c3n2)CC1] | N-(4-(4-((3-methyl-4-((1- methyl-1H- benzo[d][1,2,3]triazol-5- yl)oxy)phenyl)amino)pyrido[3,2- d]pyrimidin-6- yl)cyclohexyl)acrylamide | 535.3 (M + H) |
| 401 | 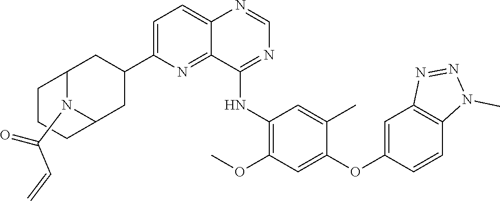 [SMILES: C=CC(=O)N1C2CCCC1CC(c1ccc3ncnc(Nc4cc(C)c(Oc5ccc6c(c5)nnn6C)cc4OC)c3n1)C2] | 1-(3-(4-((2-methoxy-5-methyl-4- ((1-methyl-1H- benzo[d][1,2,3]triazol-5- yl)oxy)phenyl)amino)pyrido[3,2- d]pyrimidin-6-yl)-9- azabicyclo[3.3.1]nonan-9- yl)prop-2-en-1-one trifluoroacetatexc | 591.3 (M + H − TFA) |
| 402 | 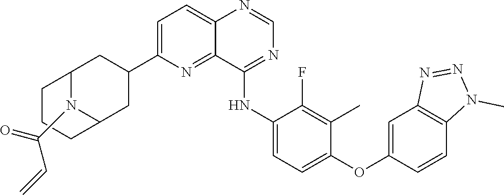 [SMILES: C=CC(=O)N1C2CCCC1CC(c1ccc3ncnc(Nc4ccc(Oc5ccc6c(c5)nnn6C)c(C)c4F)c3n1)C2] | 1-(3-(4-((2-fluoro-3-methyl-4- ((1-methyl-1H- benzo[d][1,2,3]triazol-5- yl)oxy)phenyl)amino)pyrido[3,2- d]pyrimidin-6-yl)-9- azabicyclo[3.3.1]nonan-9- yl)prop-2-en-1-one trifluoroacetate | 579.3 (M + H − TFA) |

### Example 99

### 1-(4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridin-1(2H)-yl)prop-2-en-1-one (compound 109)

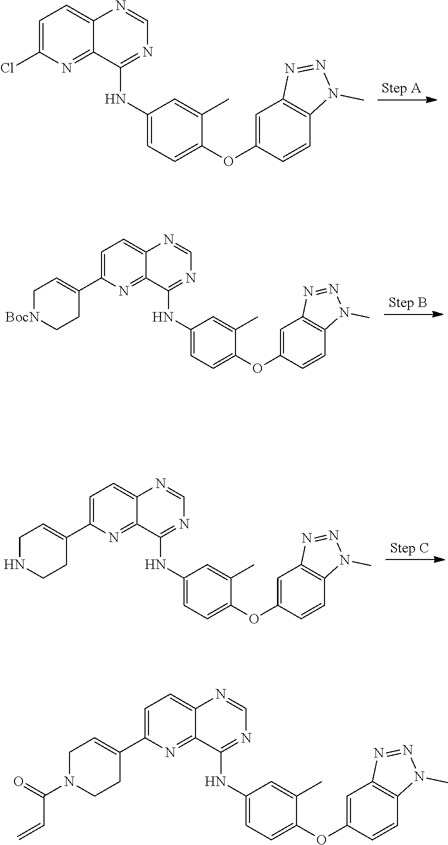 [SMILES: Cc1cc(Nc2ncnc3ccc(Cl)nc23)ccc1Oc1ccc2c(c1)nnn2C ; Cc1cc(Nc2ncnc3ccc(C4=CCN(C(=O)OC(C)(C)C)CC4)nc23)ccc1Oc1ccc2c(c1)nnn2C ; Cc1cc(Nc2ncnc3ccc(C4=CCNCC4)nc23)ccc1Oc1ccc2c(c1)nnn2C ; C=CC(=O)N1CC=C(c2ccc3ncnc(Nc4ccc(Oc5ccc6c(c5)nnn6C)c(C)c4)c3n2)CC1]

Step A. tert-butyl 4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridine-1(2H)-carboxylate. To a slurry of 6-chloro-N-(3-methyl-4-((1-methyl-1H-benzo[d][1,2,3]triazol-5-yl)oxy)phenyl)pyrido[3,2-d]pyrimidin-4-amine (100 mg, 0.24 mmol), K₂CO₃ (132 mg, 0.96 mmol), and tert-butyl 4-(4,4,5,5-tetramethyl-1,3,2-dioxaborolan-2-yl)-3,6-dihydropyridine-1(2H)-carboxylate (81.4 mg, 0.26 mmol) in 1,4-dioxane (1.00 mL) and water (0.25 mL) was added Pd(PPh₃)₄ (27.7 mg, 23.9 μmol). The reaction vessel was sealed and heated to 100° C. for 3 h. The mixture was filtered over celite and concentrated in vacuo. The crude reaction was purified by silica gel chromatography (0% to 20% MeOH/DCM) to afford tert-butyl 4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridine-1(2H)-carboxylate (140.4 mg, 88% yield). LCMS (MM-ES+APCI, Pos): m/z 565.3 (M+H).

Step B. N-(3-methyl-4-((1-methyl-1H-benzo[d][1,2,3]triazol-5-yl)oxy)phenyl)-6-(1,2,3,6-tetrahydropyridin-4-yl)pyrido[3,2-d]pyrimidin-4-amine. To a solution of tert-butyl 4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridine-1(2H)-carboxylate (45 mg, 79 μmol) in DCM (1.00 mL) was added TFA (0.24 mL). The mixture was stirred 1 h, concentrated in vacuo, and purified by preparative HPLC (0% to 100% MeCN/0.1% aqueous TFA). The product was neutralized with sat. aq. NaHCO₃ and extracted with 20% MeOH/DCM to afford N-(3-methyl-4-((1-methyl-1H-benzo[d][1,2,3]triazol-5-yl)oxy)phenyl)-6-(1,2,3,6-tetrahydropyridin-4-yl)pyrido[3,2-d]pyrimidin-4-amine (19 mg, 52% yield). LCMS (MM-ES+APCI, Pos): m/z 465.2 (M+H).

Step C. 1-(4-(4-((3-methyl-4-((1-methyl-H-benzo[d][1,2,3]triazol-5-yl)oxy)phenyl)amino)pyrido[3,2-d]pyrimidin-6-yl)-3,6-dihydropyridin-1(2H)-yl)prop-2-en-1-one 2,2,2-trifluoroacetate. A slurry of N-(3-methyl-4-((1-methyl-1H-benzo[d][1,2,3]triazol-5-yl)oxy)phenyl)-6-(1,2,3,6-tetrahydropyridin-4-yl)pyrido[3,2-d]pyrimidin-4-amine (19 mg, 41 μmol) in THF (1 mL) was cooled to −78° C., after which TEA (5.7 μL, 41 μmol) was added. The solution was stirred for 5 min and acryloyl chloride (4.0 μL, 49 μmol) was added. After 15 min at −78° C., the reaction was removed from the dry ice bath, stirred for 15 min, and quenched with sat. aq. NaHCO₃ (5 drops). The mixture was warmed to 25° C. and concentrated in vacuo. The crude residue was purified by silica gel chromatography (0% to 20% MeOH/DCM) and preparative HPLC (0% to 100% MeCN/0.1% aqueous TFA) to afford 1-(4-(4-((3-methyl-4-((1-methyl-1H-benzo[d][1,2,3]triazol-5-yl)oxy)phenyl)amino)pyrido[3,2-d]pyrimidin-6-yl)-3,6-dihydropyridin-1(2H)-yl)prop-2-en-1-one 2,2,2-trifluoroacetate (8.7 mg, 32%). LCMS (MM-ES+APCI, Pos): m/z 519.3 (M+H-TFA).

Compounds 109, 110, 403-414 in Table 28 were prepared according to Example 99 steps A to C. For step C, fluoroacrylamides were synthesized using HATU and fluoroacrylic acid as described in Example 56 Step C.

TABLE 28
A list of compounds 109, 110, 403-414

| | | | MS |
|---|---|---|---|
| Cpd. | | | API |
| ID | Structure | Chemical Name | (m/z) |
| 109 | 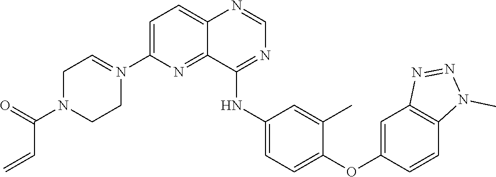 | 1-(4-(4-((3-methyl-4-((1-methyl- 1H-benzo[d][1,2,3]triazol-5- yl)oxy)phenyl)amino)pyrido[3,2- d]pyrimidin-6-yl)-3,6- dihydropyridin-1(2H)-yl)prop-2- en-1-one trifluoroacetate | 519.3 (M + H − TFA) |
| 110 | 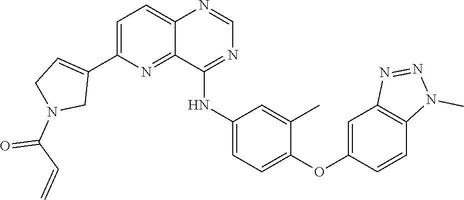 [SMILES: C=CC(=O)N1CC=C(c2ccc3ncnc(Nc4ccc(Oc5ccc6c(c5)nnn6C)c(C)c4)c3n2)C1] | 1-(3-(4-((3-methyl-4-((1-methyl- 1H-benzo[d][1,2,3]triazol-5- yl)oxy)phenyl)amino)pyrido[3,2- d]pyrimidin-6-yl)-2,5-dihydro-1H- pyrrol-1-yl)prop-2-en-1-one | 505.2 (M + H) |
| 403 | 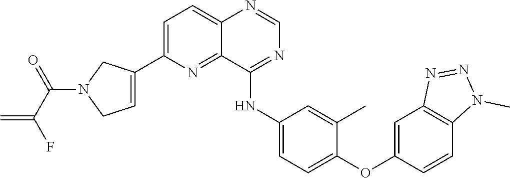 [SMILES: C=C(F)C(=O)N1CC=C(c2ccc3ncnc(Nc4ccc(Oc5ccc6c(c5)nnn6C)c(C)c4)c3n2)C1] | 2-fluoro-1-(3-(4-((3-methyl-4-((1- methyl-1H-benzo[d][1,2,3]triazol- 5-yl)oxy)phenyl)amino)pyrido[3,2- d]pyrimidin-6-yl)-2,5-dihydro-1H- pyrrol-1-yl)prop-2-en-1-one | 523.2 (M + H) |
| 404 | 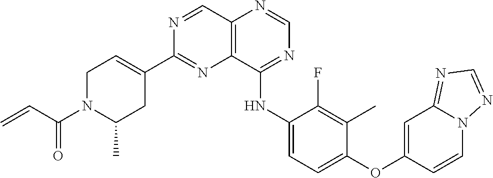 [SMILES: C=CC(=O)N1CC=C(c2ncc3ncnc(Nc4ccc(Oc5ccn6ncnc6c5)c(C)c4F)c3n2)C[C@@H]1C] | (S)-1-(4-(8-((4-([1,2,4]triazolo[1,5- a]pyridin-7-yloxy)-2-fluoro-3- methylphenyl)amino)pyrimido[5,4- d]pyrimidin-2-yl)-2-methyl-3,6- dihydropyridin-1(2H)-yl)prop-2- en-1-one | 538.1 (M + H) |
| 405 | 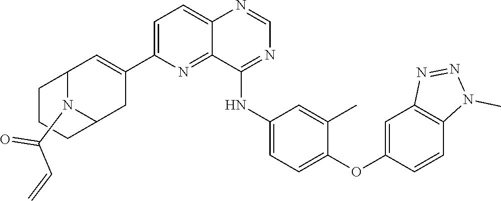 [SMILES: C=CC(=O)N1C2C=C(c3ccc4ncnc(Nc5ccc(Oc6ccc7c(c6)nnn7C)c(C)c5)c4n3)CC1CCC2] | 1-(3-(4-((3-methyl-4-((1-methyl- 1H-benzo[d][1,2,3]triazol-5- yl)oxy)phenyl)amino)pyrido[3,2- d]pyrimidin-6-yl)-9- azabicyclo[3.3.1]non-2-en-9- yl)prop-2-en-1-one | 559.3 (M + H) |
| 406 | 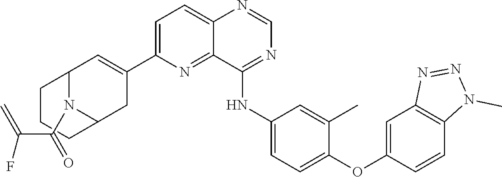 [SMILES: C=C(F)C(=O)N1C2C=C(c3ccc4ncnc(Nc5ccc(Oc6ccc7c(c6)nnn7C)c(C)c5)c4n3)CC1CCC2] | 2-fluoro-1-(3-(4-((3-methyl-4-((1- methyl-1H-benzo[d][1,2,3]triazol- 5-yl)oxy)phenyl)amino)pyrido[3,2- d]pyrimidin-6-yl)-9- azabicyclo[3.3.1]non-2-en-9- yl)prop-2-en-1-one trifluoroacetate | 577.3 (M + H − TFA) |
| 407 | 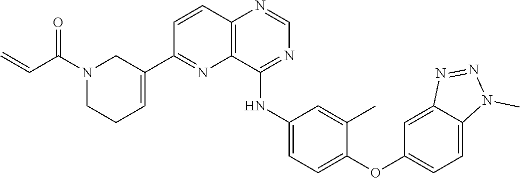 [SMILES: C=CC(=O)N1CCC=C(c2ccc3ncnc(Nc4ccc(Oc5ccc6c(c5)nnn6C)c(C)c4)c3n2)C1] | 1-(5-(4-((3-methyl-4-((1-methyl- 1H-benzo[d][1,2,3]triazol-5- yl)oxy)phenyl)amino)pyrido[3,2- d]pyrimidin-6-yl)-3,6- dihydropyridin-1(2H)-yl)prop-2- en-1-one | 519.2 (M + H) |
| 408 | 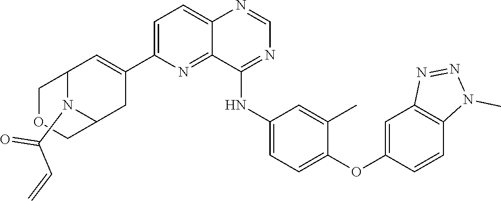 [SMILES: C=CC(=O)N1C2C=C(c3ccc4ncnc(Nc5ccc(Oc6ccc7c(c6)nnn7C)c(C)c5)c4n3)CC1COC2] | 1-(7-(4-((3-methyl-4-((1-methyl- 1H-benzo[d][1,2,3]triazol-5- yl)oxy)phenyl)amino)pyrido[3,2- d]pyrimidin-6-yl)-3-oxa-9- azabicyclo[3.3.1]non-6-en-9- yl)prop-2-en-1-one trifluoroacetate | 561.3 (M + H − TFA) |
| 409 | 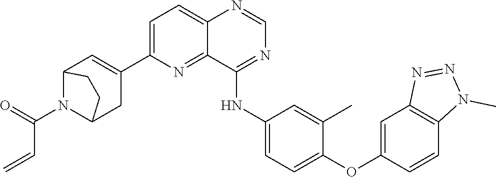 [SMILES: C=CC(=O)N1C2C=C(c3ccc4ncnc(Nc5ccc(Oc6ccc7c(c6)nnn7C)c(C)c5)c4n3)CC1CC2] | 1-(3-(4-((3-methyl-4-((1-methyl- 1H-benzo[d][1,2,3]triazol-5- yl)oxy)phenyl)amino)pyrido[3,2- d]pyrimidin-6-yl)-8- azabicyclo[3.2.1]oct-2-en-8- yl)prop-2-en-1-one trifluoroacetate | 545.3 (M + H − TFA) |
| 410 | 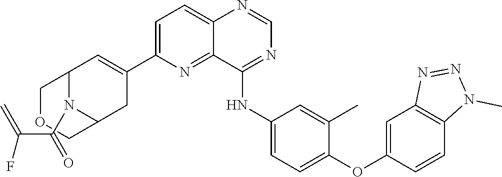 [SMILES: C=C(F)C(=O)N1C2C=C(c3ccc4ncnc(Nc5ccc(Oc6ccc7c(c6)nnn7C)c(C)c5)c4n3)CC1COC2] | 2-fluoro-1-(7-(4-((3-methyl-4-((1- methyl-1H-benzo[d][1,2,3]triazol- 5-yl)oxy)phenyl)amino)pyrido[3,2- d]pyrimidin-6-yl)-3-oxa-9- azabicyclo[3.3.1]non-6-en-9- yl)prop-2-en-1-one trifluoroacetate | 579.3 (M + H − TFA) |
| 411 | 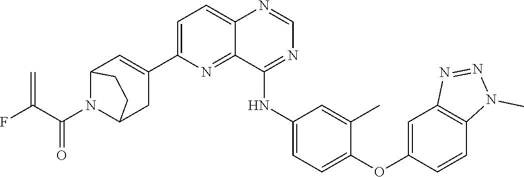 [SMILES: C=C(F)C(=O)N1C2C=C(c3ccc4ncnc(Nc5ccc(Oc6ccc7c(c6)nnn7C)c(C)c5)c4n3)CC1CC2] | 2-fluoro-1-(3-(4-((3-methyl-4-((1- methyl-1H-benzo[d][1,2,3]triazol- 5-yl)oxy)phenyl)amino)pyrido[3,2- d]pyrimidin-6-yl)-8- azabicyclo[3.2.1]oct-2-en-8- yl)prop-2-en-1-one trifluoroacetate | 563.3 (M + H − TFA) |
| 412 | 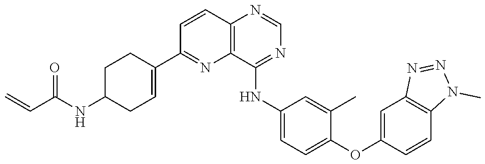 [SMILES: C=CC(=O)NC1CC=C(c2ccc3ncnc(Nc4ccc(Oc5ccc6c(c5)nnn6C)c(C)c4)c3n2)CC1] | N-(4-(4-((3-methyl-4-((1-methyl- 1H-benzo[d][1,2,3]triazol-5- yl)oxy)phenyl)amino)pyrido[3,2- d]pyrimidin-6-yl)cyclohex-3-en-1- yl)acrylamide | 533.2 (M + H) |
| 413 | 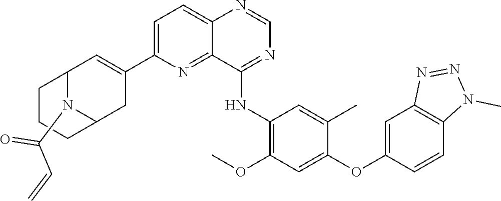 [SMILES: C=CC(=O)N1C2C=C(c3ccc4ncnc(Nc5cc(C)c(Oc6ccc7c(c6)nnn7C)cc5OC)c4n3)CC1CCC2] | 1-(3-(4-((2-methoxy-5-methyl-4- ((1-methyl-1H- benzo[d][1,2,3]triazol-5- yl)oxy)phenyl)amino)pyrido[3,2- d]pyrimidin-6-yl)-9- azabicyclo[3.3.1]non-2-en-9- yl)prop-2-en-1-one trifluoroacetate | 589.3 (M + H − TFA) |
| 414 | 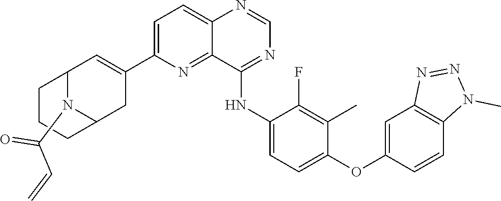 [SMILES: C=CC(=O)N1C2C=C(c3ccc4ncnc(Nc5ccc(Oc6ccc7c(c6)nnn7C)c(C)c5F)c4n3)CC1CCC2] | 1-(3-(4-((2-fluoro-3-methyl-4-((1- methyl-1H-benzo[d][1,2,3]triazol- 5-yl)oxy)phenyl)amino)pyrido[3,2- d]pyrimidin-6-yl)-9- azabicyclo[3.3.1]non-2-en-9- yl)prop-2-en-1-one | 577.3 (M + H) |

### Biological Assays

### ErbB Enzyme Assays

In vitro enzyme assays based on CisBio HTRF-KinEASE-TK technology were used to determine compound potencies. EGFR WT (Invitrogen, cat #PR7295B) at 0.025 nM or ErbB2 V777L (Signal Chem, cat #E27-12IG-100) were incubated with 250 nM TK-substrate-biotin (CisBio, part of cat #62TKOPEC) along with test compounds in 50 mM HEPES, pH 7.5, 10 mM MgCl₂, 1 mM EDTA, 0.01% Brij-35 in a final volume of 10 μL. The buffer contained 100 nM SEB (CisBio) for ErbB2 V777L, however SEB was not included for EGFR WT. The ATP concentration was at the Kₘ for each enzyme (25 μM for EGFR WT, 110 μM for ErbB2 V777L). Compounds were prepared as a three-fold serial dilution in DMSO and added to the assay to give the appropriate final concentration. After a 1 h incubation at ambient temperature, the reaction was quenched by adding 10 μL of 62.5 nM Sa-XL665 and 0.25×TK-Ab-Cryptate in HTRF detection buffer (all from CisBio, part of cat #62TKOPEC). After another 1 h incubation at ambient temperature, the extent of substrate phosphorylation was determined using a PerkinElmer EnVision multimode plate reader via time-resolved fluorescence dual wavelength detection. The percent of control (POC) was calculated using the ratio of emission at 655 nm to the emission at 620 nm. One hundred POC was determined using DMSO only controls (no test compounds present) and zero POC was determined using pre-quenched controls reactions. A 4-parameter logistic equation was fit to the POC values as a function of the test compound concentration and the IC50 value was determined as the point where the curve crossed 50 POC.

### ErbB Cellular Phosphorylation Assays

HEK-293 cells were engineered to constitutively express ErbB2 mutant L755S or EGFR wild type (constructs obtained from GenScript, Piscataway, NJ). Cells were seeded in poly-D-lysine coated, 96-well plates at a density of 50,000 cells/well in complete medium (DMEM supplemented with 10% fetal bovine serum and G418) and incubated for 24 h at 37° C., 5% CO₂ prior to treatment. Cells were treated with compound using three-fold dilutions at final concentrations ranging from 5 mM to 0.00025 mM for 1 h at 37° C., 5% CO₂. EGFR-WT cells were stimulated with 500 ng/mL recombinant human EGF (R&D Systems, 236-EG) for an additional 5 min. After compound incubation, medium was removed from the plate, and cells were lysed in buffer containing protease and phosphatase inhibitors (100 mL/well). Cell lysates were analyzed by enzyme-linked immunosorbent assay (ELISA). The ELISAs were performed to measure phosphorylated ErbB2 or phosphorylated EGFR levels, respectively (R&D Systems, DYC-1768, Human Phospho-ErbB2 DuoSet ELISA; DYC1095, Human Phospho-EGFR DuoSet IC ELISA) according to the manufacturer's instructions. Absorbance was measured at 450 nm using a BioTek Cytation 5 plate reader (Agilent, Santa Clara, CA) and IC₅₀ values were calculated using a 4-parameter fit.

TABLE 9
ErbB Enzyme Assay Results
A < 50 nM ≤ B < 100 nM ≤ C < 500 nM ≤ D < 1000 nM ≤ E

| Compound | V777L IC₅₀ | EGFR IC₅₀ |
|---|---|---|
| ID | (nM) | (nM) |
| 1 | B | C |
| 2 | B | C |
| 3 | B | C |
| 4 | A | A |
| 5 | A | C |
| 6 | C | C |
| 7 | A | A |
| 8 | A | A |
| 9 | C | D |
| 10 | B | C |
| 11 | A | C |
| 12 | C | C |
| 13 | C | C |
| 14 | E | E |
| 15 | D | E |
| 16 | C | C |
| 17 | C | D |
| 18 | B | C |
| 19 | C | C |
| 20 | D | C |
| 21 | A | A |
| 22 | C | C |
| 23 | E | E |
| 24 | A | C |
| 25 | A | C |
| 26 | C | C |
| 27 | C | E |
| 28 | C | C |
| 29 | E | E |
| 30 | A | B |
| 31 | C | C |
| 32 | C | C |
| 33 | B | A |
| 34 | C | E |
| 35 | D | E |
| 36 | A | A |
| 37 | B | C |
| 38 | A | C |
| 39 | C | D |
| 40 | C | D |
| 41 | A | C |
| 42 | A | C |
| 43 | E | C |
| 44 | A | B |
| 45 | C | C |
| 46 | A | A |
| 47 | A | C |
| 48 | A | A |
| 49 | C | A |
| 50 | E | E |
| 51 | E | E |
| 52 | B | A |
| 53 | C | C |
| 54 | E | E |
| 55 | C | D |
| 56 | C | D |
| 57 | C | C |
| 58 | B | B |
| 59 | C | C |
| 60 | B | C |
| 61 | B | A |
| 62 | A | A |
| 63 | B | A |
| 64 | A | A |
| 65 | C | C |
| 66 | B | C |
| 67 | C | C |
| 68 | E | B |
| 69 | A | A |
| 70 | A | C |
| 71 | A | A |
| 72 | A | C |
| 73 | A | C |
| 74 | A | A |
| 75 | A | A |
| 76 | C | D |
| 77 | A | A |
| 78 | B | C |
| 79 | A | C |
| 80 | C | C |
| 81 | A | A |
| 82 | A | A |
| 83 | A | A |
| 84 | A | A |
| 85 | D | D |
| 86 | E | E |
| 87 | E | D |
| 88 | E | E |
| 89 | A | B |
| 90 | A | C |
| 91 | B | A |
| 92 | A | B |
| 93 | B | C |
| 94 | B | C |
| 95 | A | A |
| 96 | C | C |
| 97 | A | A |
| 98 | A | A |
| 99 | A | A |
| 100 | A | D |
| 101 | C | C |
| 102 | C | D |
| 103 | C | C |
| 104 | C | C |
| 105 | C | D |
| 106 | A | A |
| 107 | B | C |
| 108 | C | C |
| 109 | A | C |
| 110 | A | B |
| 111 | A | A |
| 112 | B | A |
| 113 | A | A |
| 114 | C | C |
| 115 | D | E |
| 116 | B | C |
| 117 | C | E |
| 118 | A | A |
| 119 | C | D |
| 120 | A | B |
| 121 | D | E |
| 122 | E | E |
| 123 | C | C |
| 124 | B | C |
| 125 | A | C |
| 126 | C | D |
| 127 | C | D |
| 128 | C | C |
| 129 | B | C |
| 130 | A | A |
| 131 | A | A |
| 132 | C | C |
| 133 | C | D |
| 134 | B | C |
| 135 | B | C |
| 136 | A | C |
| 137 | C | C |
| 138 | C | C |
| 139 | C | C |
| 140 | B | C |
| 141 | A | C |
| 142 | A | C |
| 143 | C | E |
| 144 | A | A |
| 145 | C | C |
| 146 | E | D |
| 147 | B | A |
| 148 | B | C |
| 149 | C | D |
| 150 | C | D |
| 151 | C | C |
| 152 | D | E |
| 153 | A | C |
| 154 | C | C |
| 155 | C | D |
| 156 | C | C |
| 157 | B | C |
| 158 | C | C |
| 159 | A | B |
| 160 | A | A |
| 161 | A | A |
| 162 | A | C |
| 163 | A | C |
| 164 | D | D |
| 165 | E | E |
| 166 | D | D |
| 167 | C | A |
| 168 | E | D |
| 169 | C | D |
| 170 | C | C |
| 171 | A | B |
| 172 | E | E |
| 173 | B | C |
| 174 | C | E |
| 175 | C | B |
| 176 | D | C |
| 177 | C | A |
| 178 | E | A |
| 179 | A | C |
| 180 | B | B |
| 181 | A | A |
| 182 | A | D |
| 183 | E | E |
| 184 | E | E |
| 185 | C | E |
| 186 | C | E |
| 187 | B | C |
| 188 | D | E |
| 189 | C | D |
| 190 | A | C |
| 191 | B | B |
| 192 | E | D |
| 193 | E | E |
| 194 | A | A |
| 195 | E | C |
| 196 | C | D |
| 197 | D | C |
| 198 | A | C |
| 199 | A | A |
| 200 | A | A |
| 201 | E | E |
| 202 | C | D |
| 203 | E | E |
| 204 | C | B |
| 205 | E | E |
| 206 | B | C |
| 207 | C | C |
| 208 | E | E |
| 209 | B | B |
| 210 | B | C |
| 211 | E | E |
| 212 | B | C |
| 213 | E | E |
| 214 | C | D |
| 215 | E | E |
| 216 | A | B |
| 217 | A | A |
| 218 | D | E |
| 219 | E | E |
| 220 | A | A |
| 221 | B | A |
| 222 | B | B |
| 223 | C | D |
| 224 | B | C |
| 225 | C | D |
| 226 | C | C |
| 227 | E | E |
| 228 | C | D |
| 229 | E | C |
| 230 | C | C |
| 231 | D | D |
| 232 | D | E |
| 233 | E | E |
| 234 | A | A |
| 235 | A | C |
| 236 | A | C |
| 237 | A | A |
| 238 | C | E |
| 239 | B | D |
| 240 | C | E |
| 241 | E | E |
| 242 | D | C |
| 243 | C | C |
| 244 | A | C |
| 245 | C | D |
| 246 | B | C |
| 247 | B | D |
| 248 | E | E |
| 249 | A | A |
| 250 | C | D |
| 251 | C | C |
| 252 | A | B |
| 253 | B | C |
| 254 | A | B |
| 255 | A | B |
| 256 | C | C |
| 257 | A | A |
| 258 | C | C |
| 259 | D | E |
| 260 | C | C |
| 261 | C | A |
| 262 | A | C |
| 263 | A | C |
| 264 | D | D |
| 265 | C | C |
| 266 | B | C |
| 267 | E | E |
| 268 | B | C |
| 269 | E | E |
| 270 | B | C |
| 271 | D | E |
| 272 | C | C |
| 273 | C | D |
| 274 | D | E |
| 275 | C | C |
| 276 | A | C |
| 277 | C | C |
| 278 | C | D |
| 279 | E | D |
| 280 | C | C |
| 281 | A | A |
| 282 | C | D |
| 283 | E | E |
| 284 | D | E |
| 285 | D | E |
| 286 | C | E |
| 287 | B | C |
| 288 | A | C |
| 289 | D | C |
| 290 | C | D |
| 291 | B | C |
| 292 | E | C |
| 293 | E | C |
| 294 | C | C |
| 295 | C | C |
| 296 | C | C |
| 297 | B | C |
| 298 | B | A |
| 299 | C | E |
| 300 | C | C |
| 301 | E | D |
| 302 | A | C |
| 303 | A | A |
| 304 | C | C |
| 305 | A | C |
| 306 | C | C |
| 307 | C | C |
| 308 | C | D |
| 309 | C | C |
| 310 | A | C |
| 311 | D | E |
| 312 | C | C |
| 313 | A | A |
| 314 | B | C |
| 315 | B | C |
| 316 | B | C |
| 317 | C | C |
| 318 | A | A |
| 319 | A | A |
| 320 | C | C |
| 321 | C | D |
| 322 | E | D |
| 323 | D | C |
| 324 | C | C |
| 325 | C | C |
| 326 | B | C |
| 327 | C | C |
| 328 | A | A |
| 329 | A | B |
| 335 | B | C |
| 336 | E | E |
| 337 | E | C |
| 338 | D | C |
| 339 | C | C |
| 340 | A | B |
| 341 | E | E |
| 342 | C | D |
| 343 | E | C |
| 344 | D | C |
| 345 | C | C |
| 346 | C | C |
| 347 | E | C |
| 348 | C | B |
| 349 | B | C |
| 350 | C | C |
| 351 | C | D |
| 352 | C | C |
| 353 | C | D |
| 354 | C | D |
| 355 | C | E |
| 356 | D | E |
| 357 | B | D |
| 358 | A | B |
| 359 | A | C |
| 360 | B | C |
| 361 | A | B |
| 362 | D | E |
| 363 | A | B |
| 364 | A | C |
| 365 | E | E |
| 366 | D | E |
| 367 | A | D |
| 368 | A | B |
| 369 | A | C |
| 370 | B | C |
| 371 | A | B |
| 372 | A | C |
| 373 | A | C |
| 374 | C | D |
| 375 | A | B |
| 376 | A | C |
| 377 | C | C |
| 378 | A | B |
| 379 | A | C |
| 380 | A | A |
| 381 | A | A |
| 382 | B | E |
| 383 | A | A |
| 384 | B | E |
| 385 | C | C |
| 386 | B | C |
| 387 | B | C |
| 388 | A | A |
| 389 | A | A |
| 390 | A | A |
| 391 | D | D |
| 393 | A | A |
| 394 | A | A |
| 395 | C | C |
| 396 | B | C |
| 397 | C | D |
| 398 | B | C |
| 399 | E | E |
| 400 | C | B |
| 401 | B | C |
| 402 | C | C |
| 403 | A | B |
| 404 | C | C |
| 405 | C | D |
| 406 | E | E |
| 407 | A | A |
| 408 | A | C |
| 409 | B | C |
| 410 | E | E |
| 411 | E | E |
| 412 | C | C |
| 413 | E | E |
| 414 | D | D |

TABLE 10
ErbB Cellular Phosphorylation Assay Results
A < 100 nM ≤ B < 500 nM ≤ C < 1000 nM ≤ D < 5000 nM ≤ E

| Compound | L755S IC₅₀ | EGFR IC₅₀ |
|---|---|---|
| ID | (nM) | (nM) |
| 3 | B | E |
| 4 | A | D |
| 11 | A | E |
| 15 | C | E |
| 21 | A | D |
| 25 | B | E |
| 33 | A | E |
| 39 | B | E |
| 42 | A | E |
| 49 | B | E |
| 55 | B | E |
| 70 | A | D |
| 90 | A | E |
| 93 | A | E |
| 95 | A | E |
| 101 | B | E |
| 105 | B | E |
| 107 | B | E |
| 109 | A | E |
| 110 | B | E |

### Blood Brain Barrier Penetration Assay in CD-1 Mice

In vitro plasma and brain binding assays were carried out using the equilibrium dialysis method. EDTA-anticoagulated plasma and diluted brain homogenate (1:3 with DPBS pH 7.4) were spiked with 2 μM test compound and dialyzed against an equal volume of 100 μL 100 mM sodium phosphate buffer (pH 7.4) at 37° C. for 4 h in a slowly rotated plate. At the end of the dialysis 50 μL aliquots from the buffer side and matrix side of the dialysis device were taken into new 96-well plates. An equal volume of opposite blank matrix (buffer or matrix) in each sample was added to reach a final volume of 100 μL. All samples were processed by protein precipitation for LC/MS/MS analysis. Unbound fraction (fᵤ) of test compound was calculated by the ratio of buffer side response to the brain homogenate/plasma side response, and unbound fraction (fᵤ,pl and fᵤ,br) of test compound in non-diluted plasma and brain tissue were calculated from measured fᵤ in homogenate and plasma with the following equation: fᵤ,pl or fᵤ,br=1/D/((1/(F/T)-1)+1/D). F is the free compound concentration as determined by the calculated concentration on the buffer side of the membrane and T is the total compound concentration as determined by the calculated concentration on the matrix side of the membrane. D is the dilution factor (D equals 1 for plasma, and 4 for brain).

A short oral absorption model is an in-vivo screening model to identify brain penetration of a compound. Three male CD-1 mice were orally dosed with an appropriate vehicle. At the 1 h timepoint post-dose, blood samples were collected via cardiac puncture, placed into EDTA anti-coagulated tubes, and spiked with 1% 1 mM quinidine (aq). The samples were placed on wet ice until centrifugation at 4000×g for 10 min. Brain tissue was rinsed with saline and the liquid was drained. The brains were weighed and transferred to tubes for homogenization. Each tube contained 1 g tissue, 9 mL buffer (1:2 v/v MeOH:15 mM PBS), and a spike of 1% 1 mM quinidine (aq). All samples were stored at −80° C. prior to LC/MS/MS analysis. Standards were prepared by spiking blank plasma and brain homogenate. Aliquots (10 μL) of sample, calibration standard, quality control, dilution quality control, single blank, and double blank were added to the 96-well plate respectively. Homogenized brain tissue along with plasma samples were precipitated by adding 190 μL cold MeCN containing internal standard (the double blank sample was quenched with 190 L MeCN). The mixture was vortex-mixed for 5 min at 1000 rpm and centrifuged for 10 min at 3220×g, 4° C. An aliquot of 70 μL supernatant was transferred to another clean 96-well plate and the supernatant was directly injected for LC-MS/MS analysis. The free fraction of test compound in biological matrix was determined by using in vitro plasma and brain tissue free fraction. Kpuu brain was calculated by the equation: Kpuu brain=cone (brain)/conc (plasma)×(fᵤ brain/fᵤ plasma).

| Compound ID | Structure | Kpᵤᵤ |
|---|---|---|
| 5 | 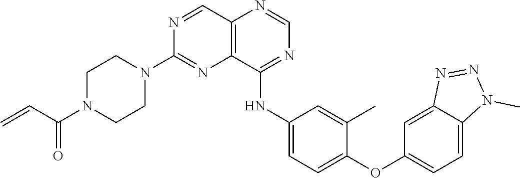 [SMILES: C=CC(=O)N1CCN(c2ncc3ncnc(Nc4ccc(Oc5ccc6c(c5)nnn6C)c(C)c4)c3n2)CC1] | 0.22 |
| Analog of compound 5 | 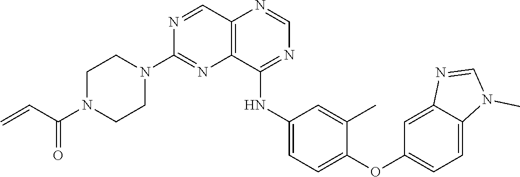 [SMILES: C=CC(=O)N1CCN(c2ncc3ncnc(Nc4ccc(Oc5ccc6c(c5)ncn6C)c(C)c4)c3n2)CC1] | 0.04 |
| 64 | 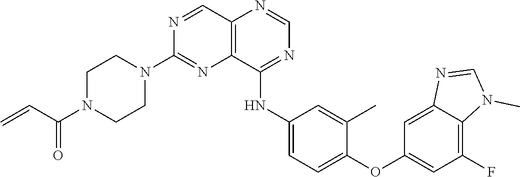 [SMILES: C=CC(=O)N1CCN(c2ncc3ncnc(Nc4ccc(Oc5cc(F)c6c(c5)ncn6C)c(C)c4)c3n2)CC1] | 0.12 |
| 235 | 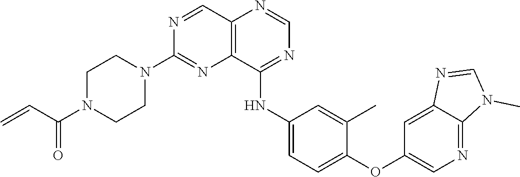 [SMILES: C=CC(=O)N1CCN(c2ncc3ncnc(Nc4ccc(Oc5cnc6c(c5)ncn6C)c(C)c4)c3n2)CC1] | 0.04 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1.-3. (canceled)

4. A compound having one of the following structures of Formula (IA-1)-(IB-1): 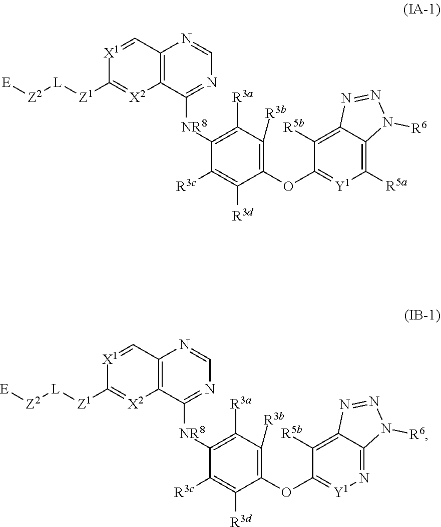
or a stereoisomer of the compound, tautomer of the compound, or a pharmaceutically acceptable salt thereof, wherein:
X¹ is CR¹ or N;
X² is CR² or N;
Y¹ is CR⁴ or N;
R¹, R², R^{3a}, R^{3b}, R^{3c}, R^{3d}, R⁴, and R^{5b} are each independently H, halo, C₁-C₃ alkyl, C₁-C₃ haloalkyl, or C₁-C₃ heteroalkyl;
R⁶ is H, C₁-C₃ alkyl, C₁-C₃ deutero-alkyl, or C₁-C₃ haloalkyl;
R⁸ and R⁹ are, each independently, H or C₁-C₃ alkyl;
Z¹ is —NR⁹—, —O—, —S—, or a direct bond;
Z² is —NR⁹— or a direct bond;
L is C₁-C₄ alkyl, C₃-C₈ cycloalkyl, C₃-C₈ heterocycloalkyl, a fused heterobicyclic, a bridged heterobicyclic, a hetero-spirocyclic, or a direct bond; and
E is α,β-unsaturated carbonyl or C₂-C₄ alkyne conjugated carbonyl.

5.-6. (canceled)

7. The compound of claim 4, wherein R⁶ is C₁-C₃ haloalkyl.

8. (canceled)

9. The compound of claim 7, wherein R⁶ is CHF₂ or CF₃.

10. The compound of claim 4, wherein
(1) Z¹ and Z² are each a direct bond;
(2) Z¹ is —NR⁹—, —O—, or —S— and Z² is a direct bond;
(3) Z¹ is a direct bond and Z² is —NR⁹—; or
(3) Z¹ and Z² are each —NR⁹—.

11.-13. (canceled)

14. The compound of claim 10, wherein R⁹ is H or CH₃.

15. (canceled)

16. The compound of claim 4, wherein R¹, R², R^{3a}, R^{3b}, R^{3c}, R^{3d}, R⁴, R^{5a}, R^{5b}, and R⁷ are, each independently, H, F, Cl, CH₃, CH₂CF₃, CF₃, CHF₂, CH₂OCH₃, OCHF₂, OCF₃, or OCH₃.

17. The compound of claim 16, wherein
R¹ and R² are, each independently, H, F, CH₃, or OCH₃;
R^{3a}, R^{3b}, R^{3c}, and R^{3d} are, each independently, H, CH₃, OCH₃, CH₂OCH₃, OCHF₂, OCF₃, F, or Cl;
R⁴ is H, F, CH₃, CH₂OCH₃, or CH₂CF₃;
R^{5a} and R^{5b} are, each independently, H, CH₃, F, or OCH₃;
R⁷ is H or CH₃; and/or
R⁸ is H or CH₃.

18.-22. (canceled)

23. The compound of claim 4, wherein the C₃-C₈ heterocycloalkyl, the fused heterobicyclic, the bridged heterobicyclic, and the hetero-spirocyclic of L have 1-3 nitrogen atoms.

24.-25. (canceled)

26. The compound of claim 4, wherein
the C₁-C₄ alkyl of L is —(CH₂)₂— or —CH₂C(CH₃)₂;
the C₃-C₈ cycloalkyl of L is 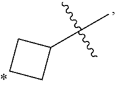 [SMILES: *C1CCC1]
wherein * indicates a location of a bond to Z²;
the C₃-C₈ heterocycloalkyl of L is azetidine, pyrrolidine, imidazolidine, pyrazolidine, piperidine, 1,2-diazinane, 1,3-diazinane, 1,4-diazinane, azapane, diazepane, or azocane;
the fused heterobicyclic of L is 3-azabicyclo[3.1.0]heptane, 2,5-diazabicyclo[4.2.0]octane, octahydropyrrolo[3.4.c]pyrrole, octahydropyrrolo[3.4.b]pyrrole, octahydro-1H-pyrrolo[3.4.c]pyridine, decahydro-2,6-naphthyridine, 2,5-diazabicyclo[4.1.0]heptane, 3,6-diazabicyclo[3.2.0]heptane, or 3,6-diazabicyclo[3.1.0]hexane;
the bridged heterobicyclic of L is 3,8-diazabicyclo[3.2.1]octane, 2,5-diazabicyclo[2.2.2]octane, 8-azabicyclo[3.2.1]octane, 3-azabicyclo[3.2.1]octane, 3,6-diazabicyclo[3.2.1]octane, 9-azabicyclo[3.3.1]nonane, 3-oxa-9-azabicyclo[3.3.1]nonane, 3-oxa-9-azabicyclo[3.3.1]non-6-ene, 9-azabicyclo[3.3.1]non-2-ene, 8-azabicyclo[3.2.1]oct-2-ene, or 3,6-diazabicyclo[3.1.1]heptane; or
the hetero-spirocyclic of L is 2,6-diazaspiro[3.3]heptane, 1,6-diazaspiro[3.3]heptane, 2-azaspiro[3.3]heptane, 1,6-diazaspiro[3.4]octane, 2,6-diazaspiro[3.4]octane, 2,7-diazaspiro[3.5]nonane, 2,8-diazaspiro[4.5]decane, 3,9-diazaspiro[5.5]undecane, 4,8-diazaspiro[2.5]octane, 5,9-diazaspiro[3.5]nonane, 6,10-diazaspiro[4.5]decane, or 1,5-diazaspiro[5.5]undecane.

27.-29. (canceled)

30. The compound of claim 4, wherein the C₃-C₈ heterocycloalkyl, the fused heterobicyclic, the bridged heterobicyclic, and the hetero-spirocyclic of L are further substituted with deuterium, halo, C₁-C₆ alkyl, or C₁-C₆ heteroalkyl.

31. (canceled)

32. The compound of claim 30, wherein the C₃-C₈ heterocycloalkyl, the fused heterobicyclic, the bridged heterobicyclic, and the hetero-spirocyclic of L are further substituted with -D, —F, —CH₃, —CF₃, —CHF₂, —CH₂F, —CH₂CHF₂, —CH₂CH₃, —CH(CH₃)₂, —CH₂OH, —CH₂OCH₃, 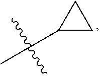 [SMILES: *C1CC1]
or —CH₂CCN.

33.-36. (canceled)

37. The compound of claim 4, wherein L has one of the following structures: 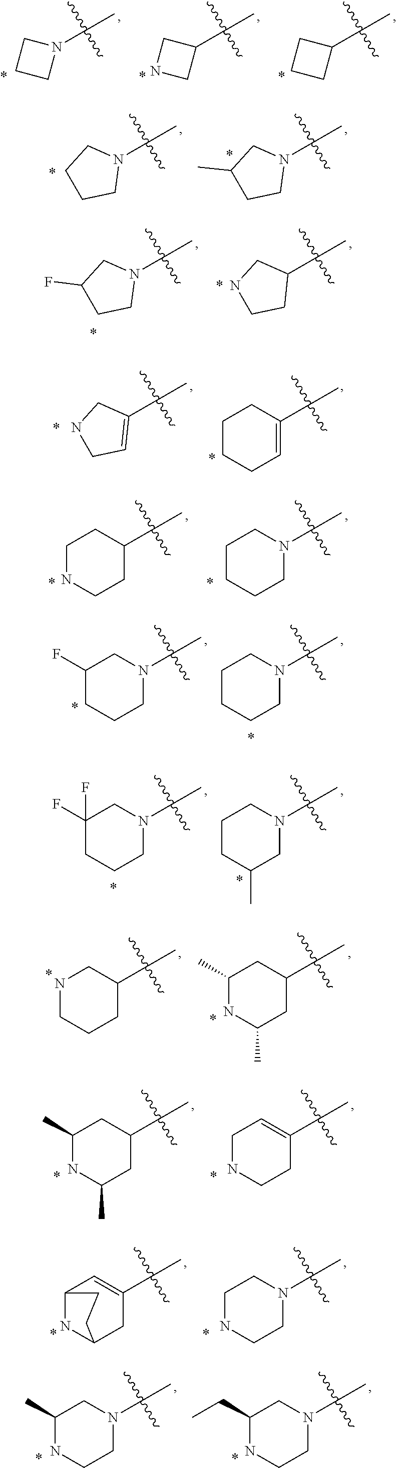 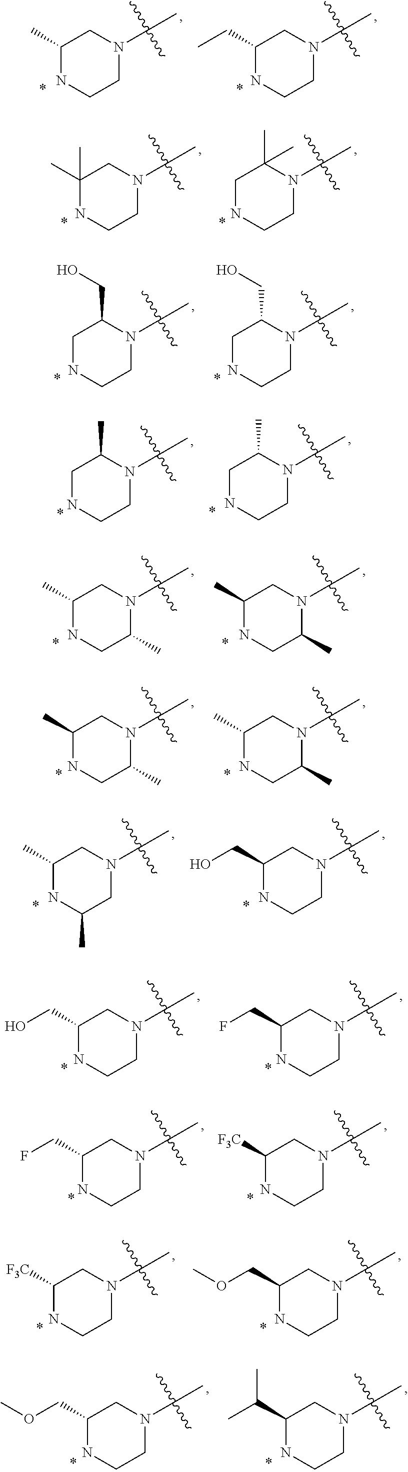 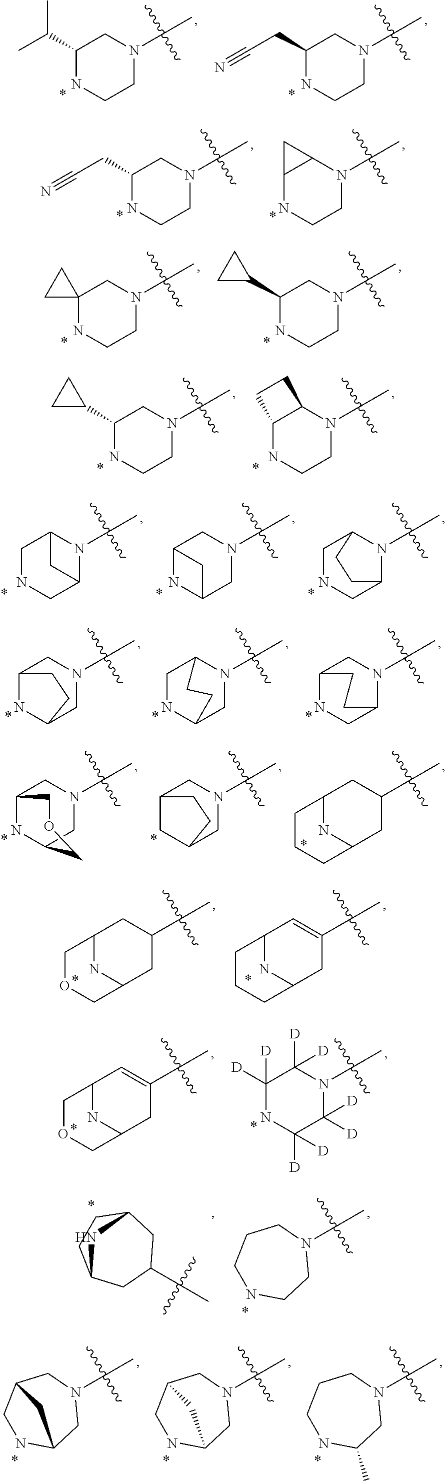 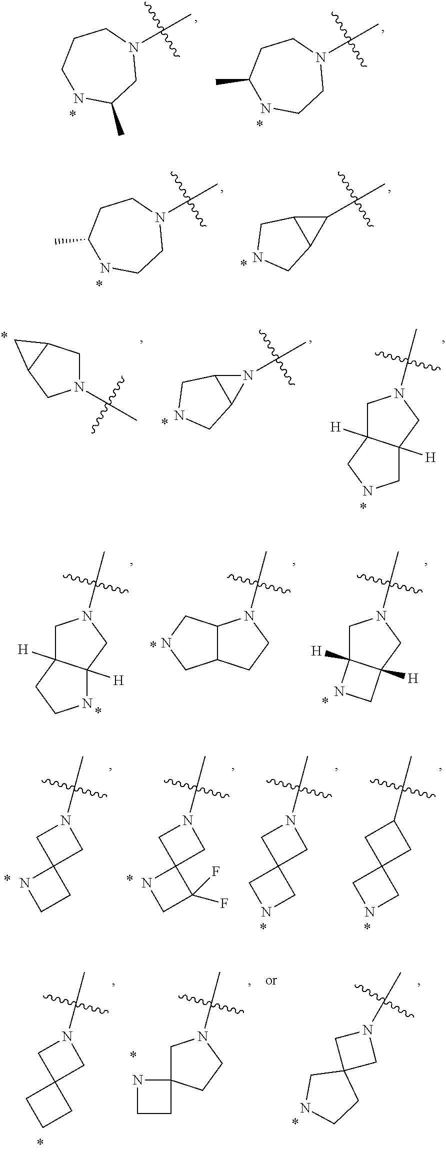
wherein * indicates a location of a bond to Z².

38. The compound of claim 4, wherein α,β-unsaturated carbonyl or C₂-C₄ alkyne conjugated carbonyl of E is further substituted with halo, C₁-C₃ alkyl, C₁-C₃ alkylhalo, C₁-C₆ heteroalkyl, —(CH₂)ₙC₃-C₇ heterocycloalkyl, or combination thereof, wherein n is an integer between 1-3.

39. The compound of claim 38, wherein E has one of the following structures:

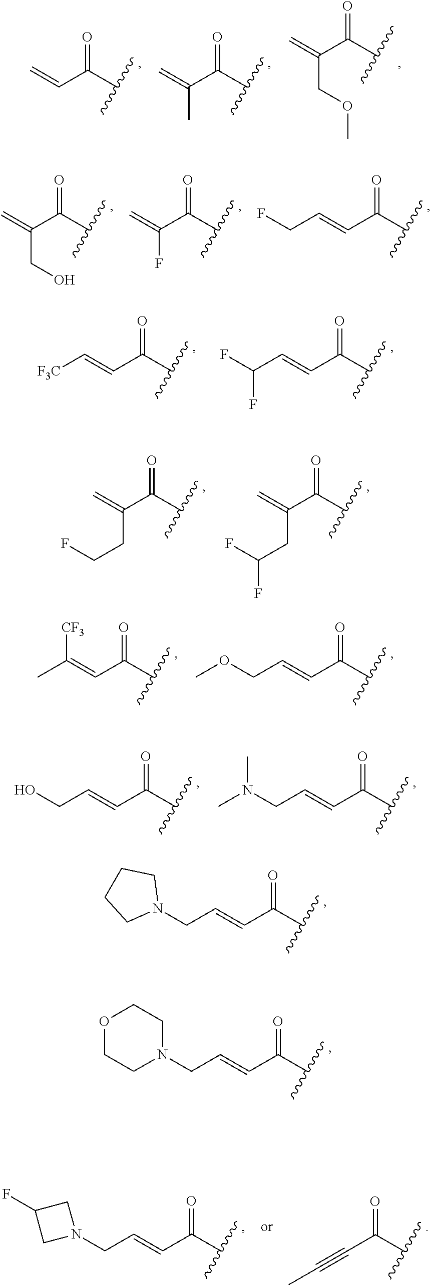

40.-42. (canceled)

43. A Compound having one of the following structures:

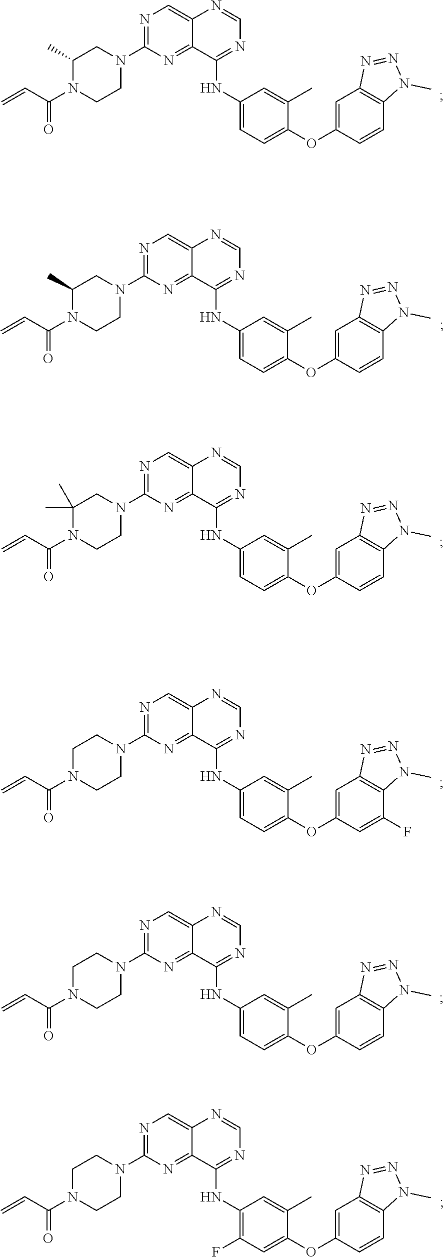

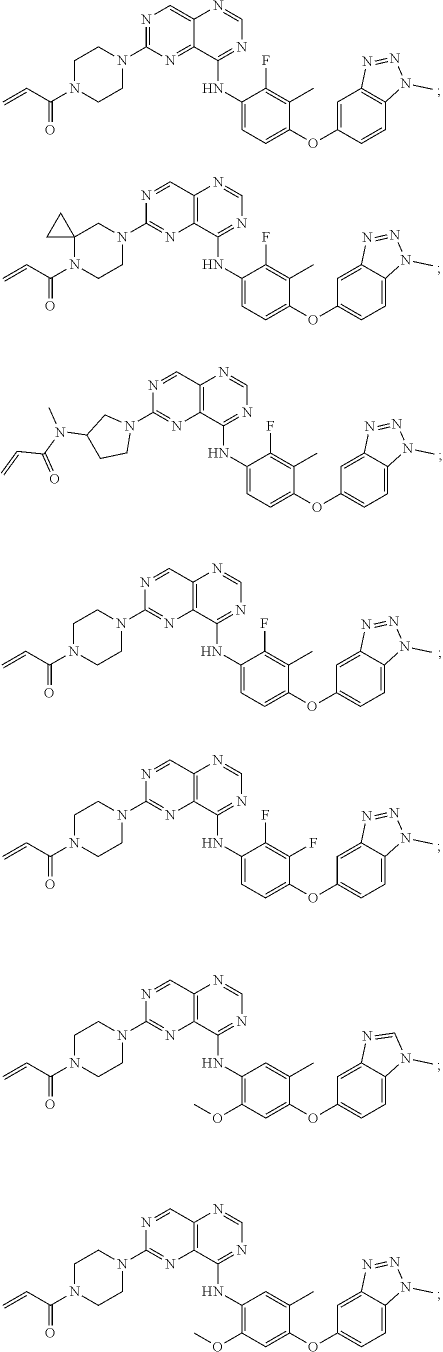

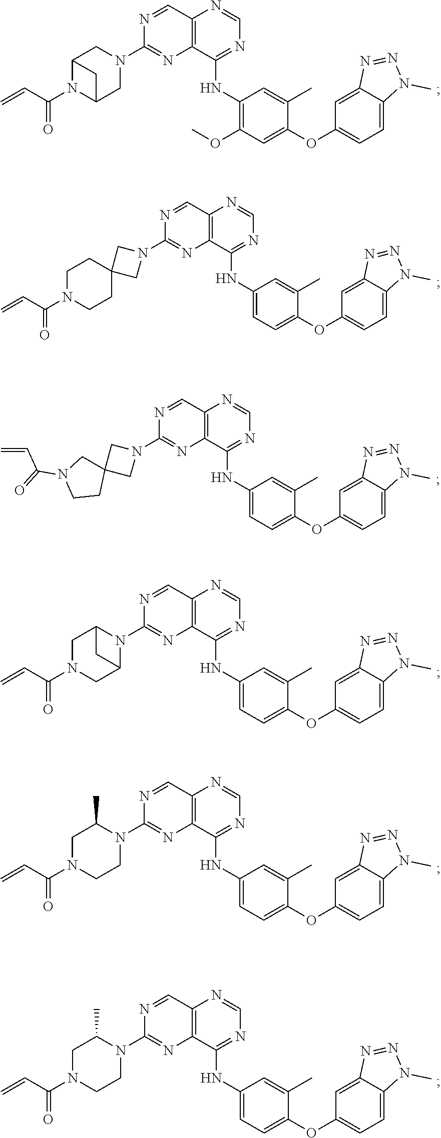

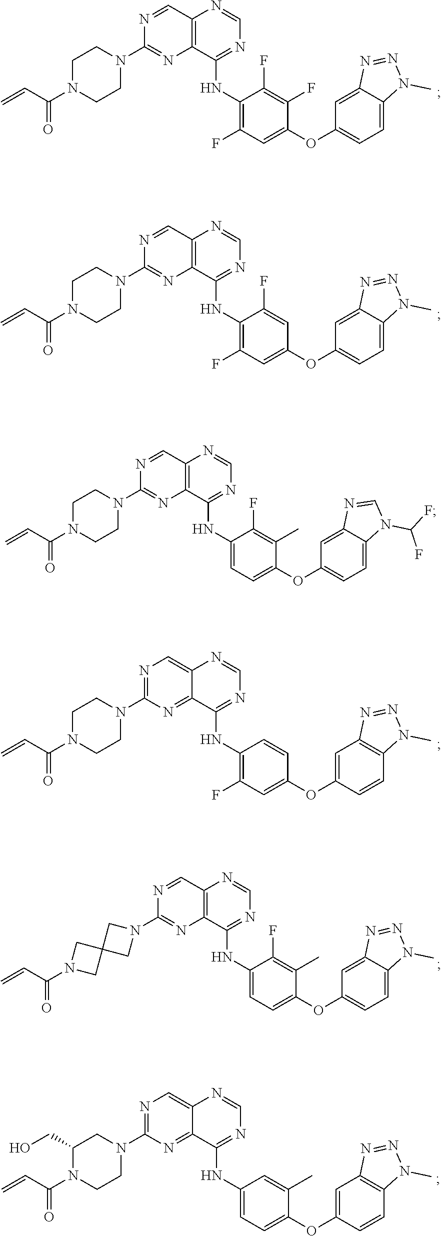

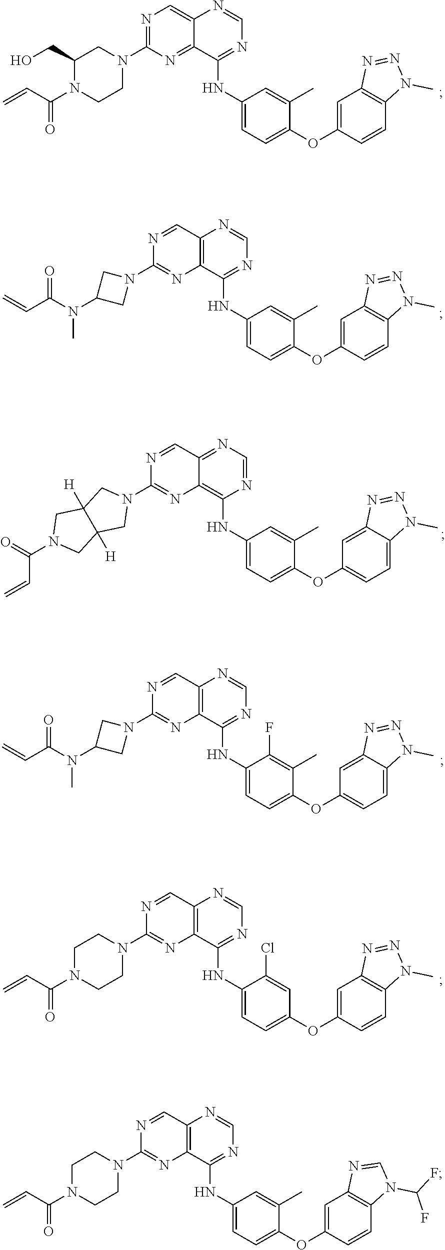

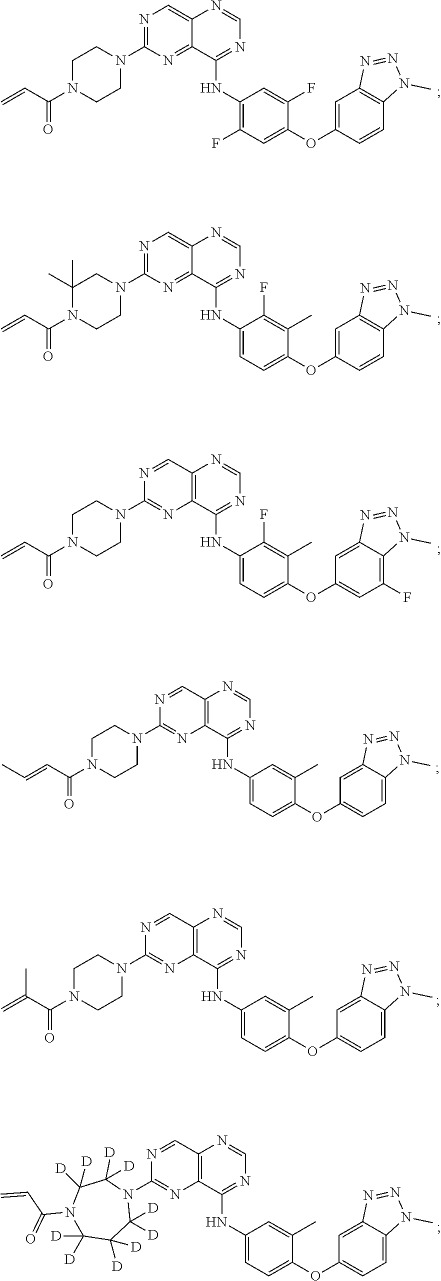

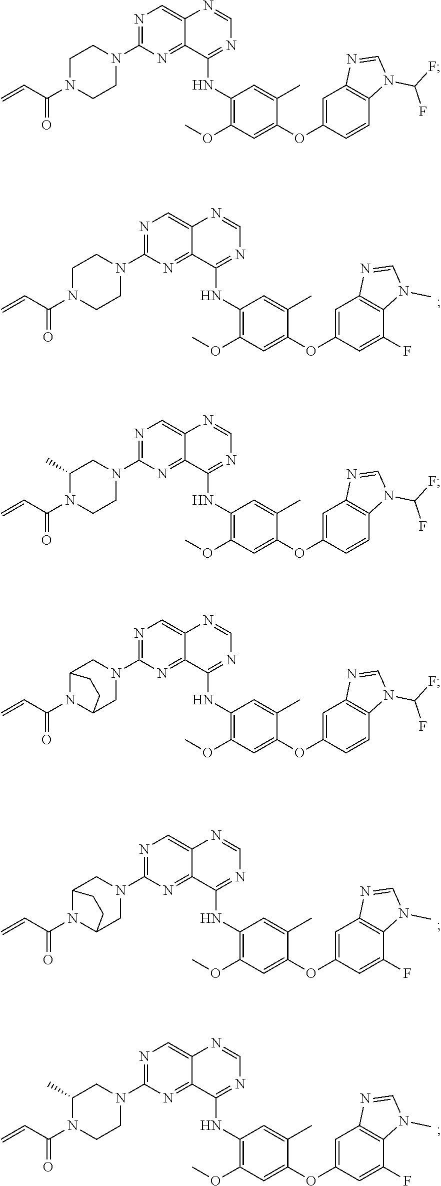

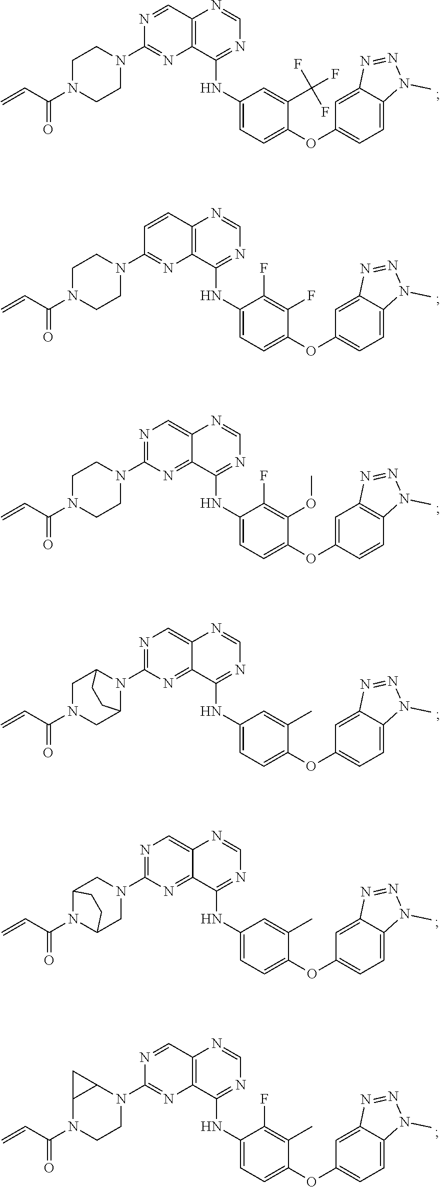

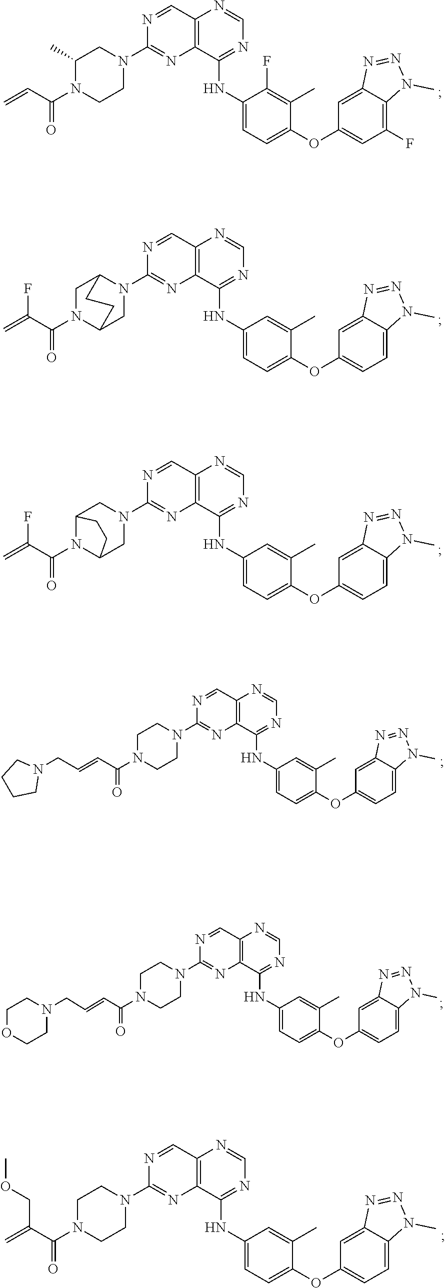

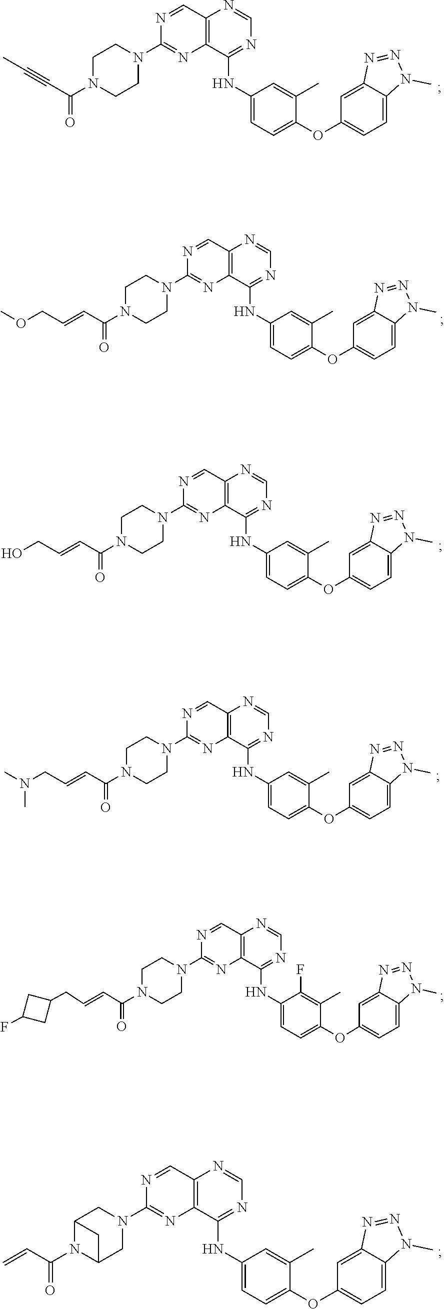

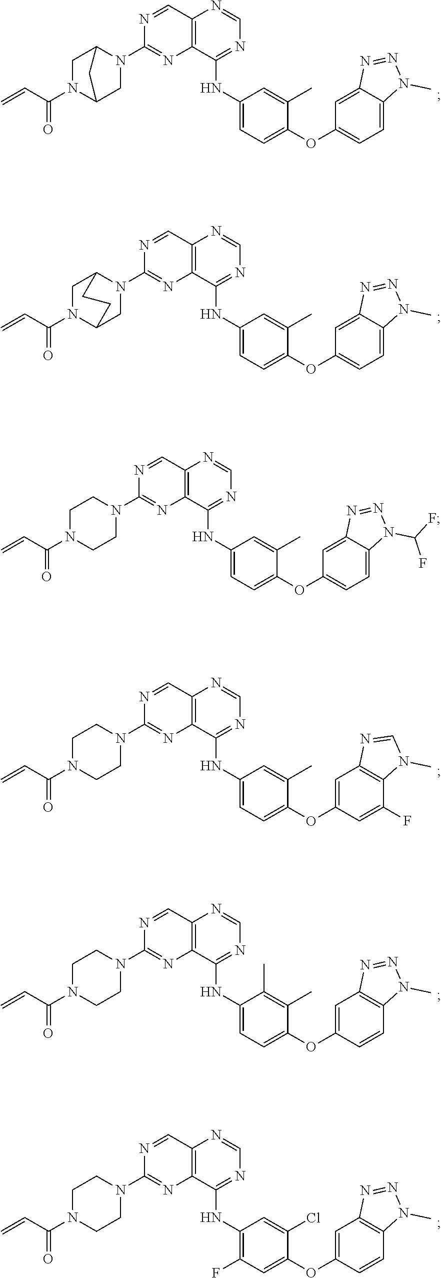

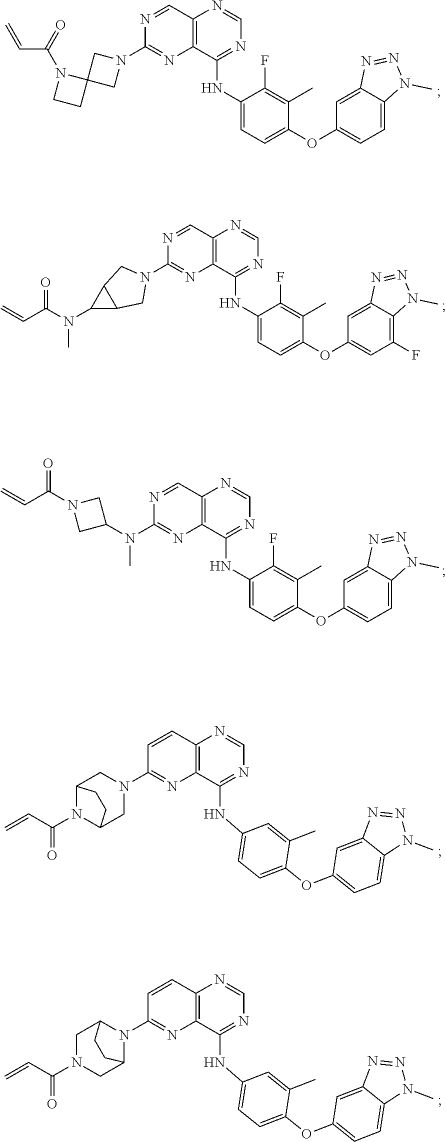

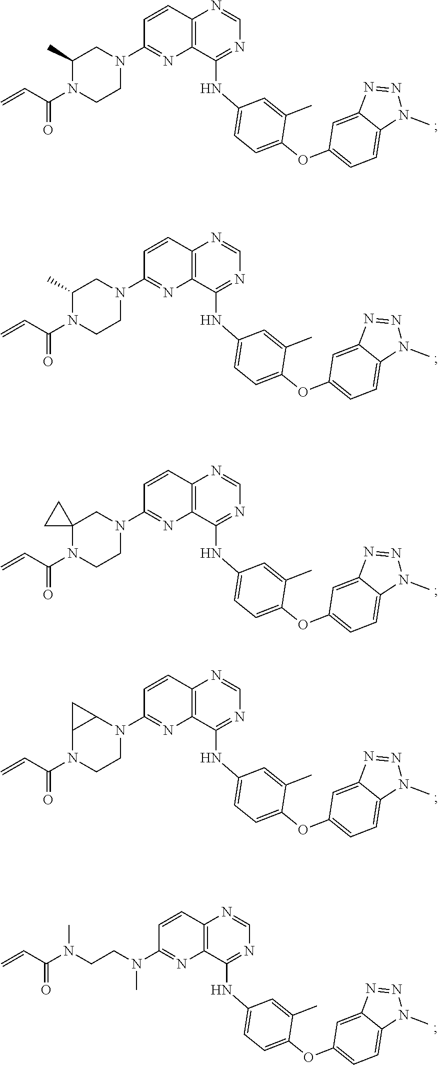

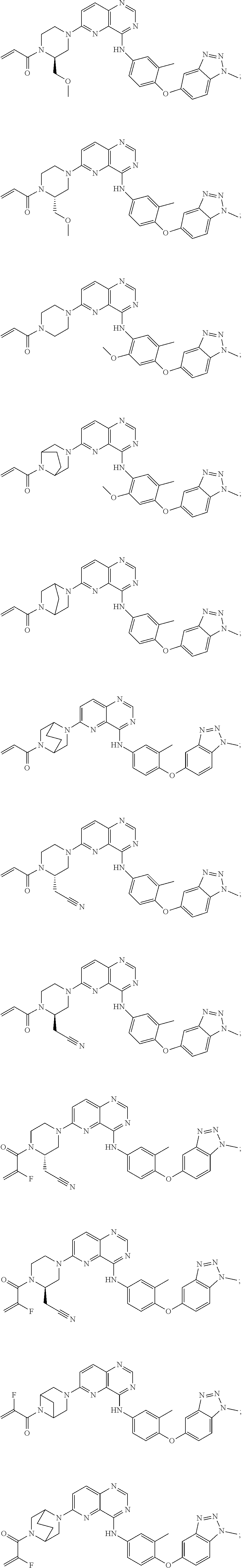

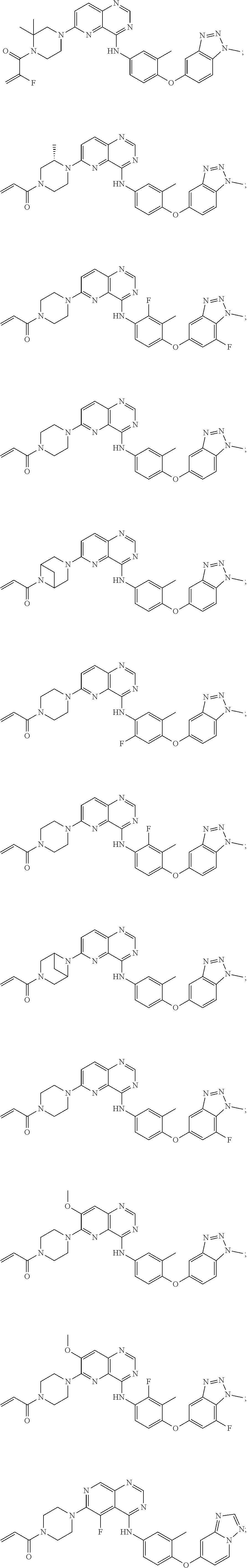

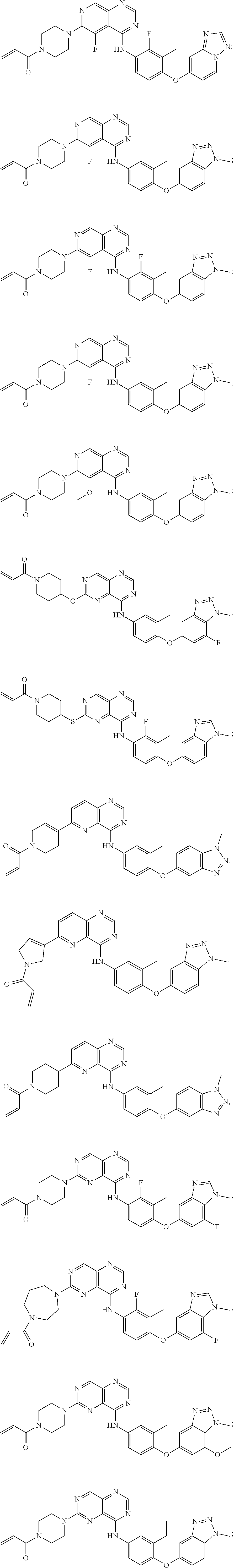

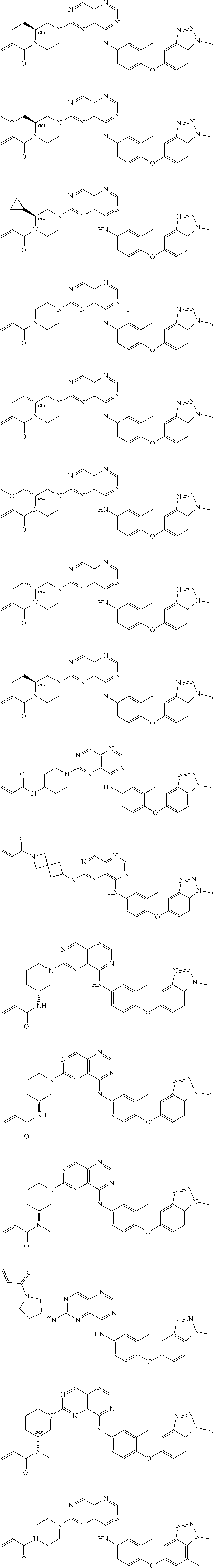

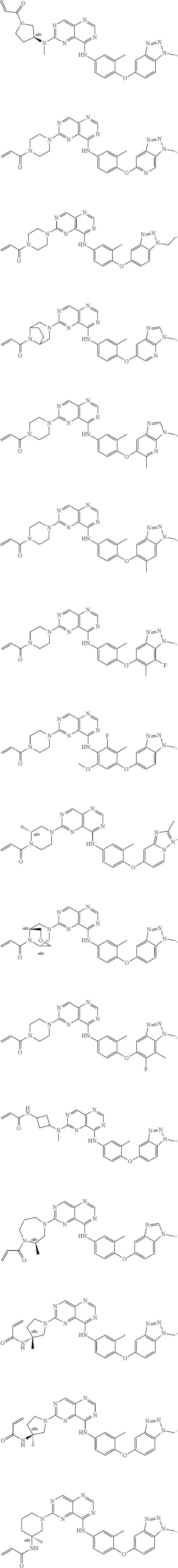

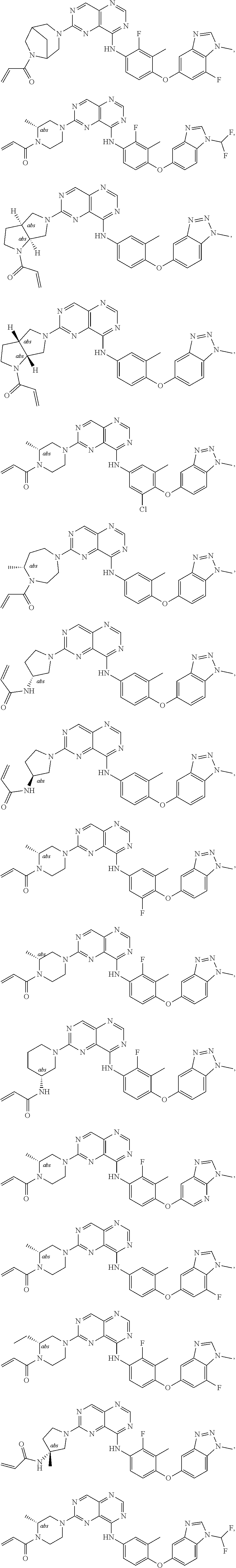

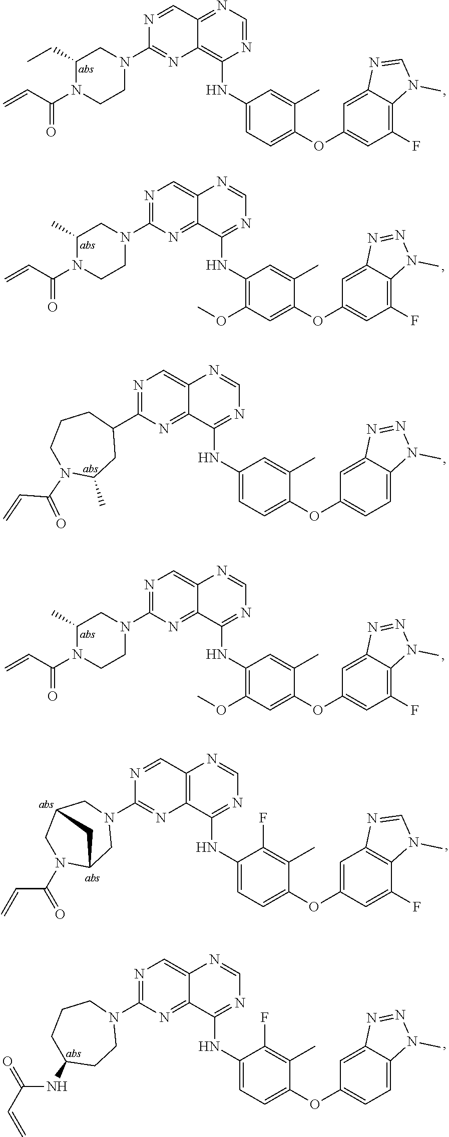

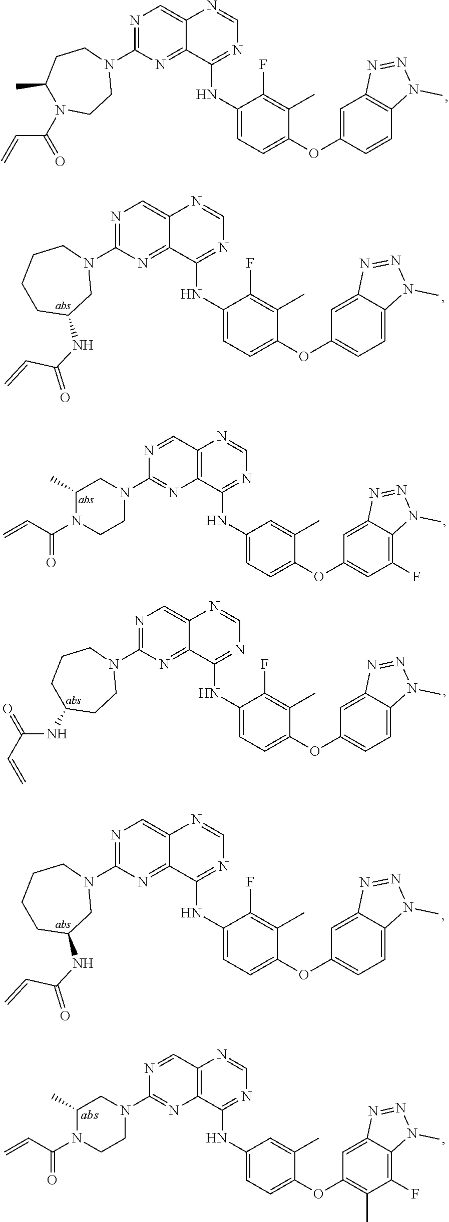

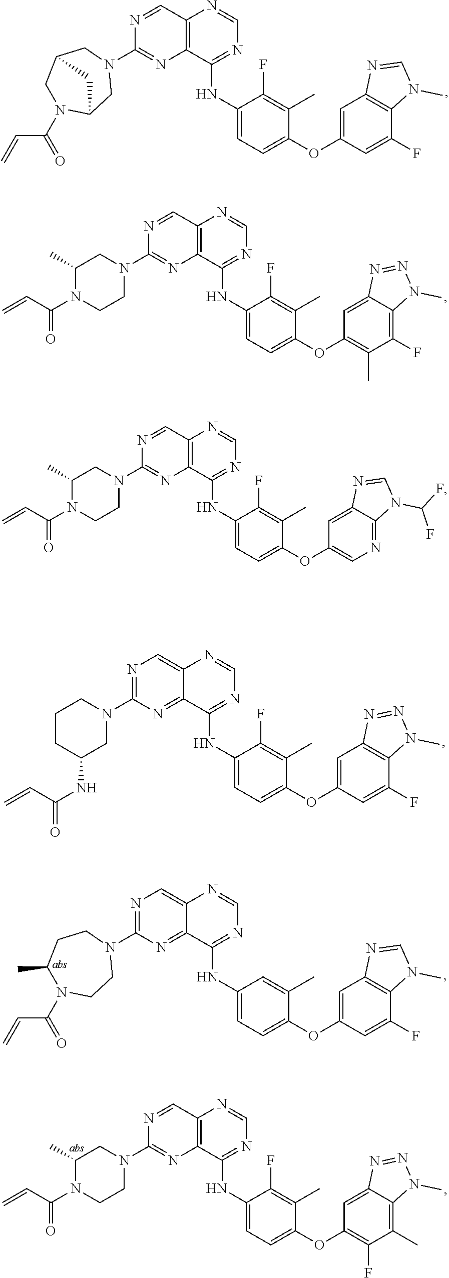

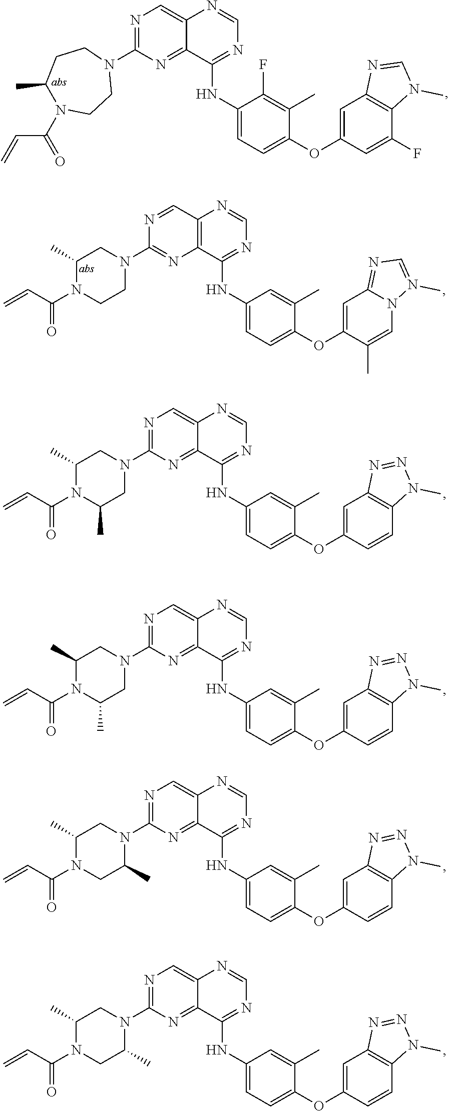

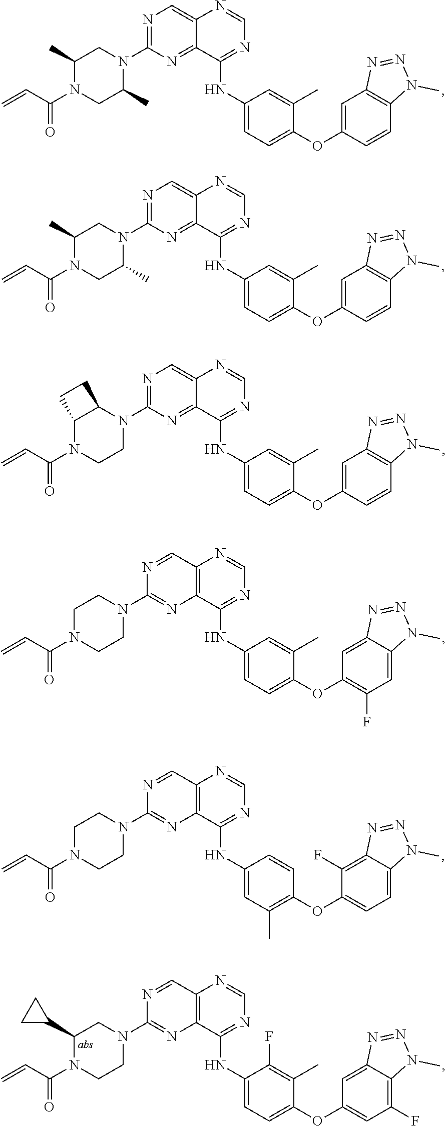

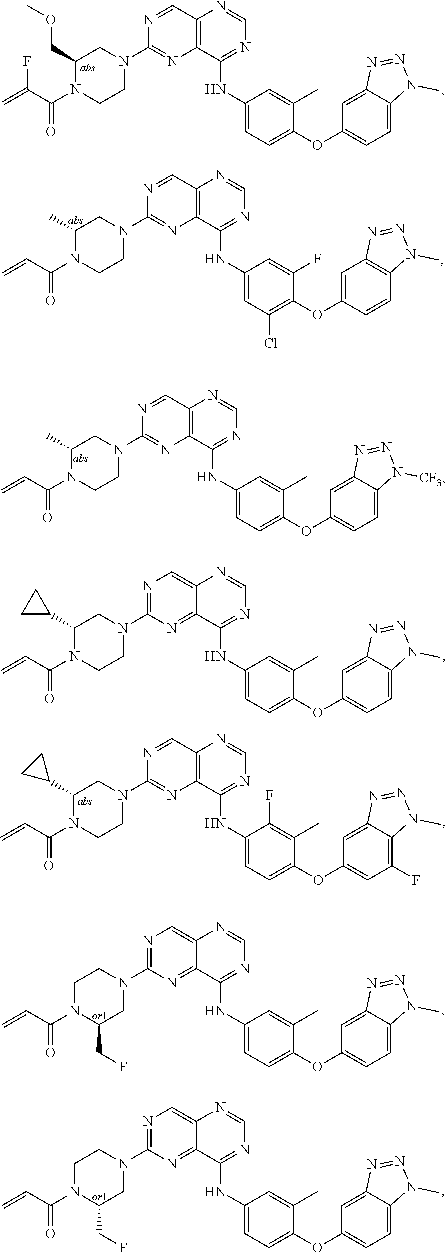

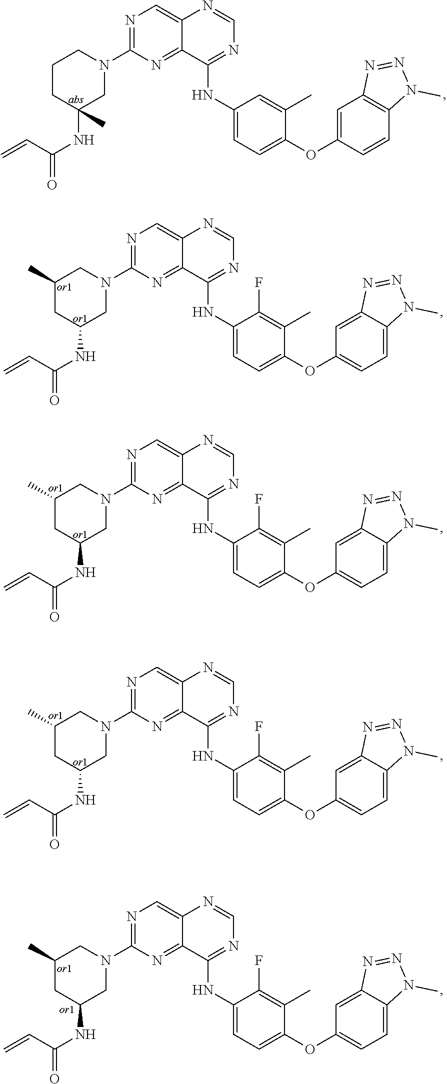

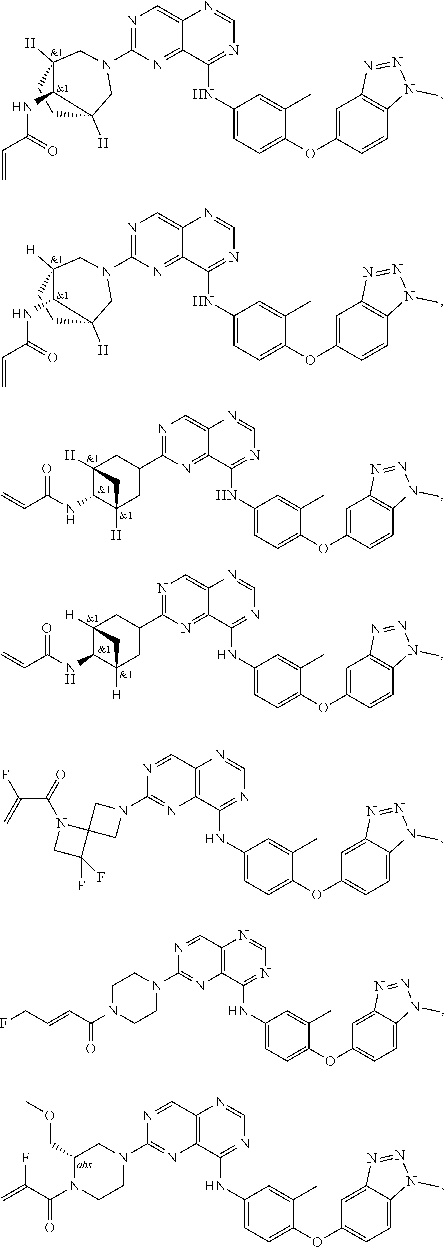

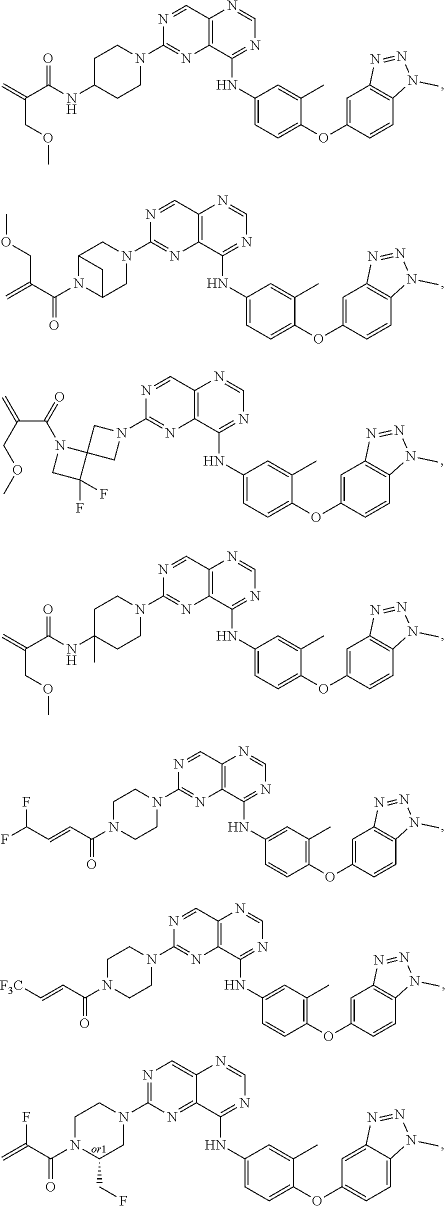

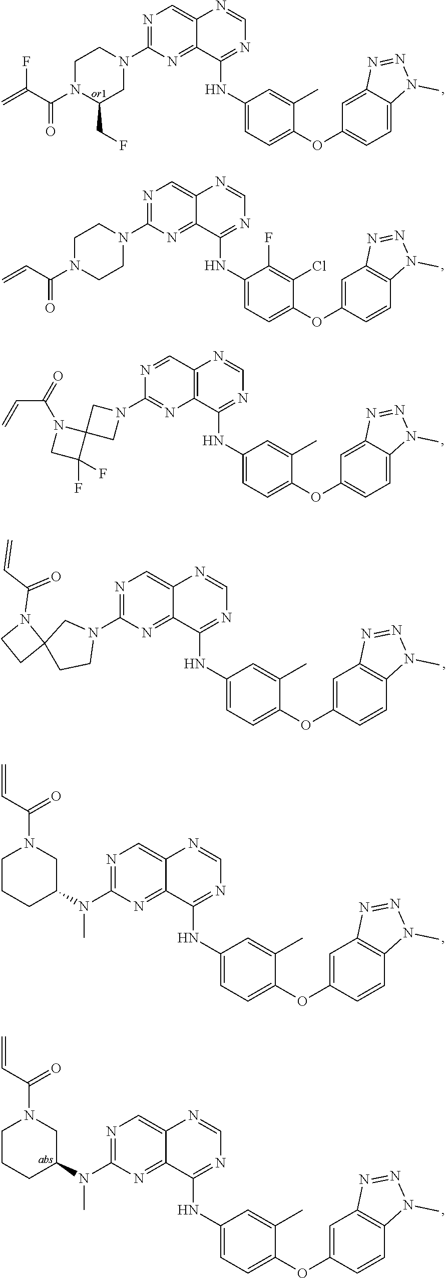

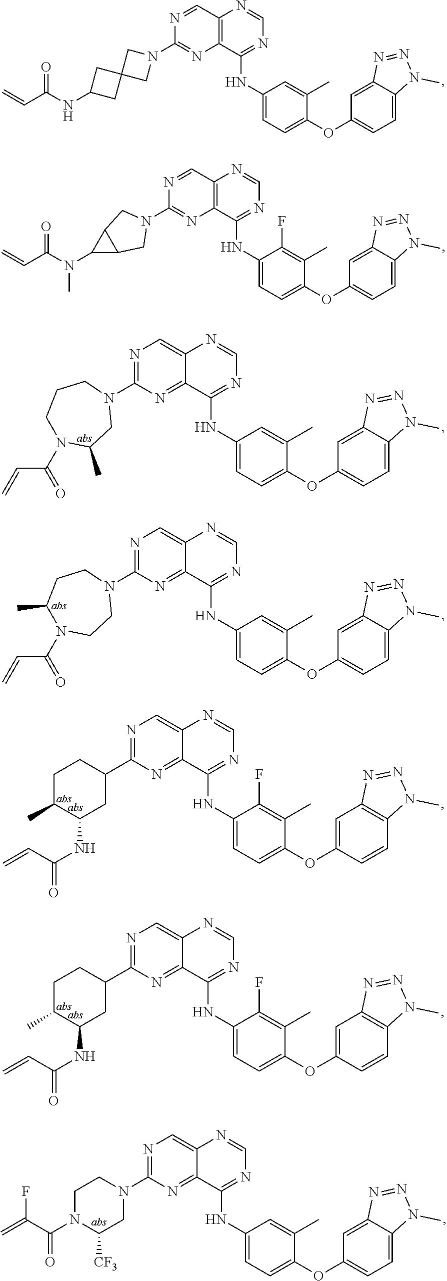

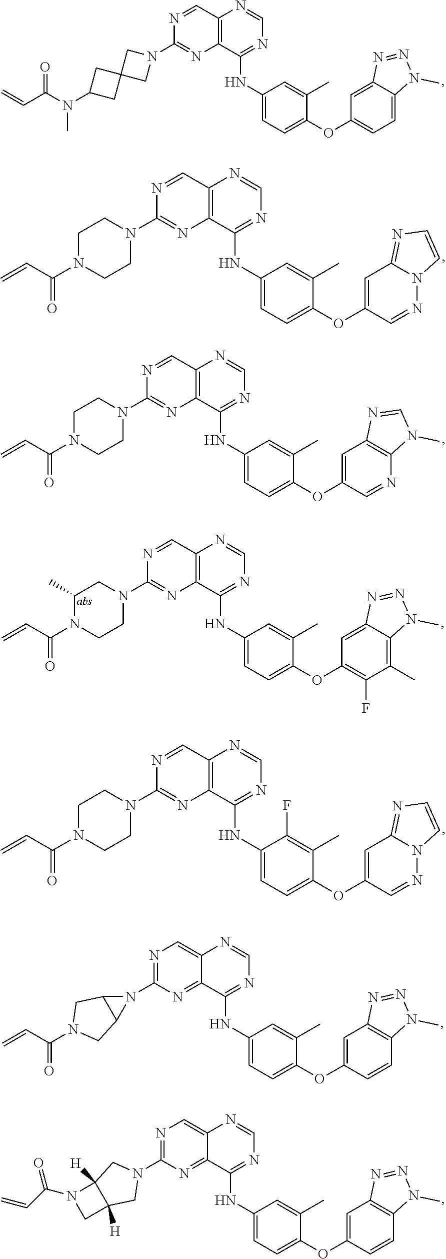

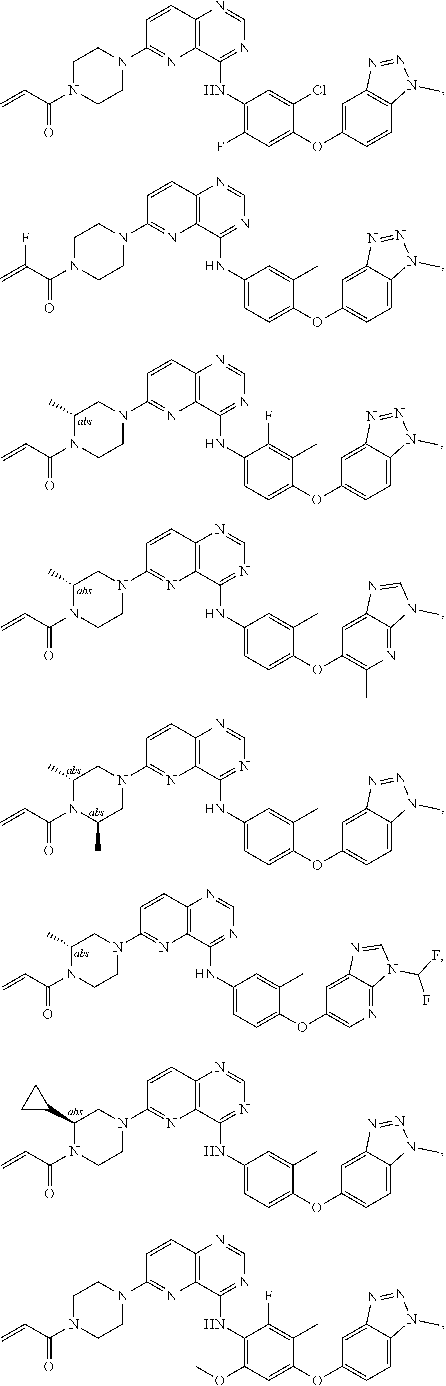

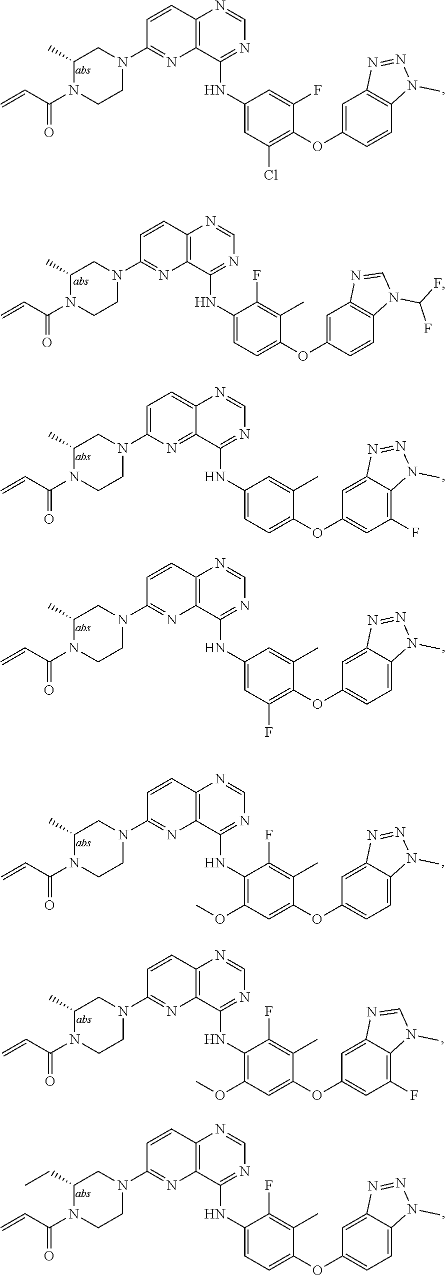

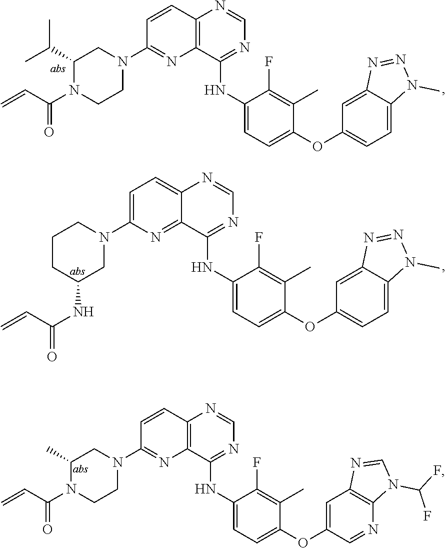

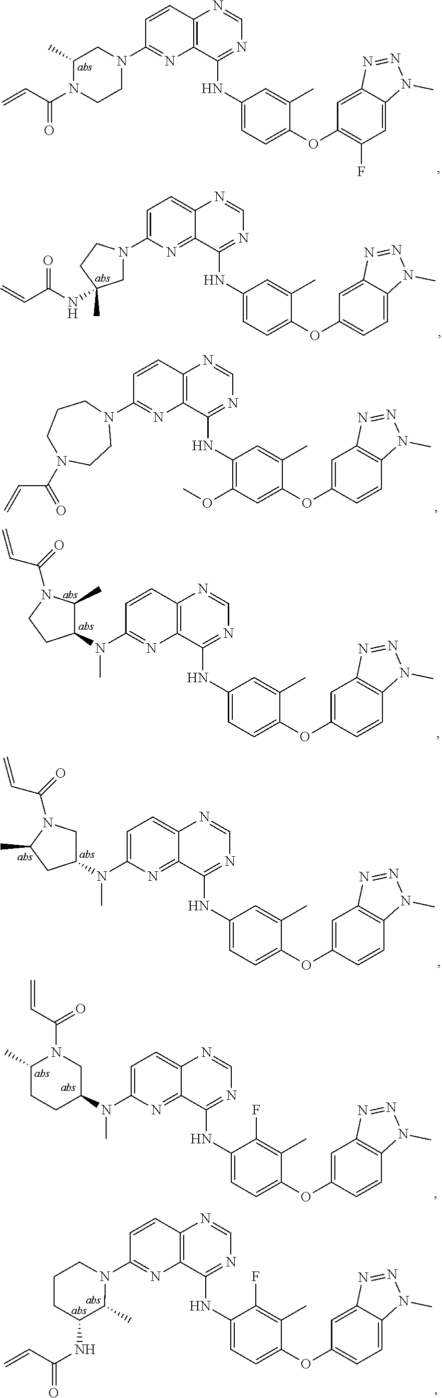

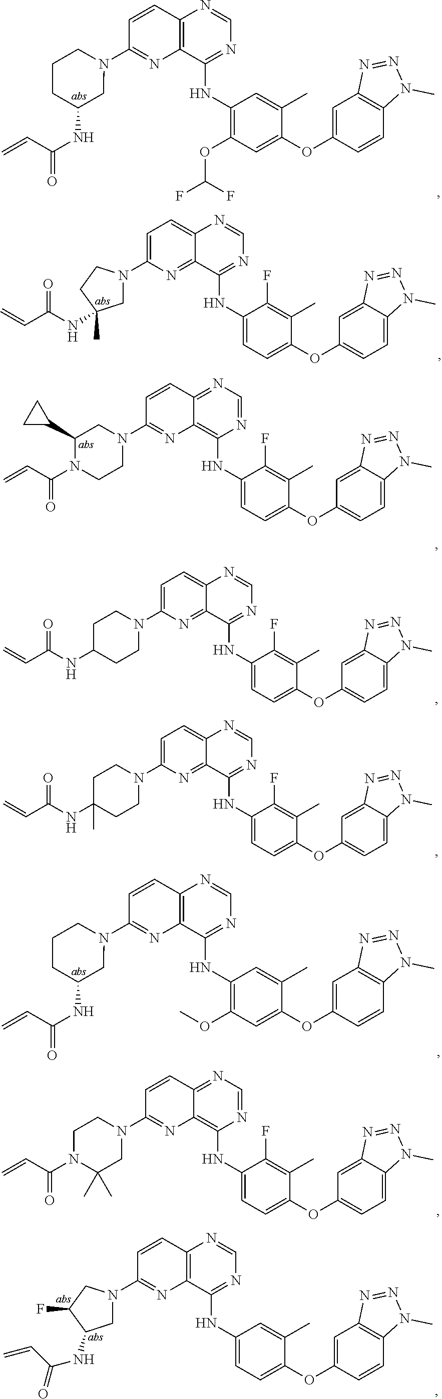

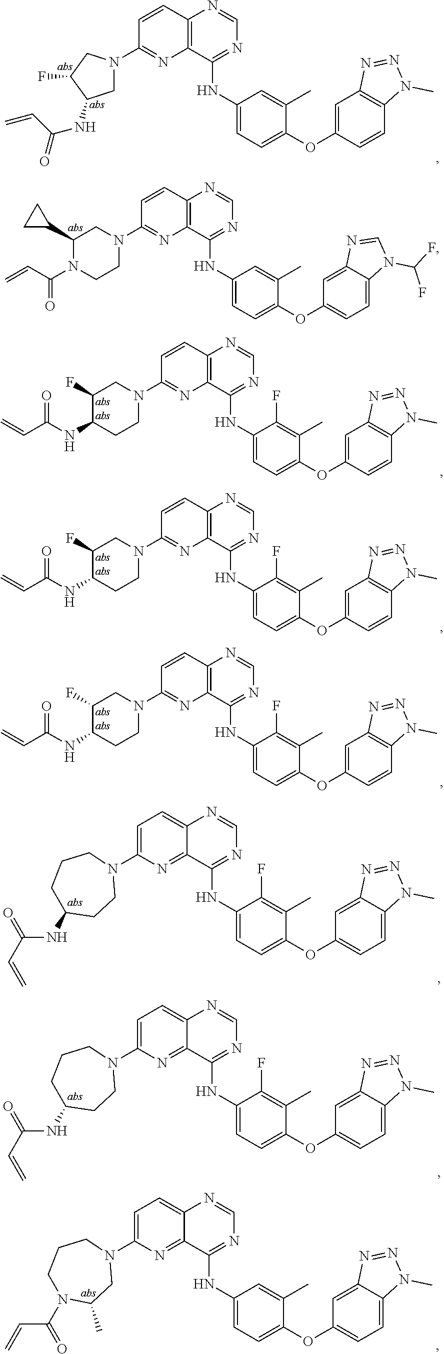

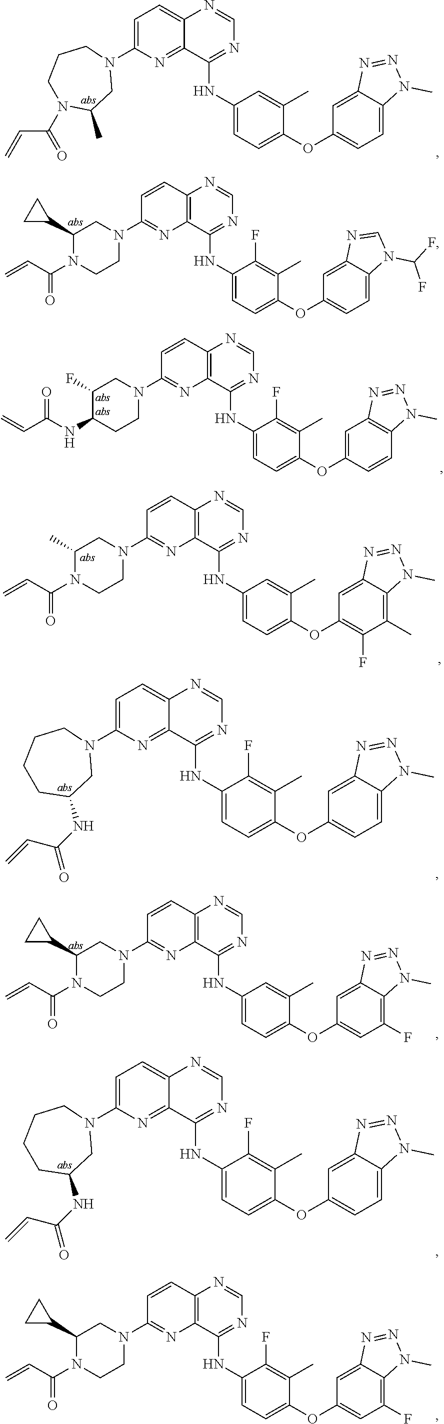

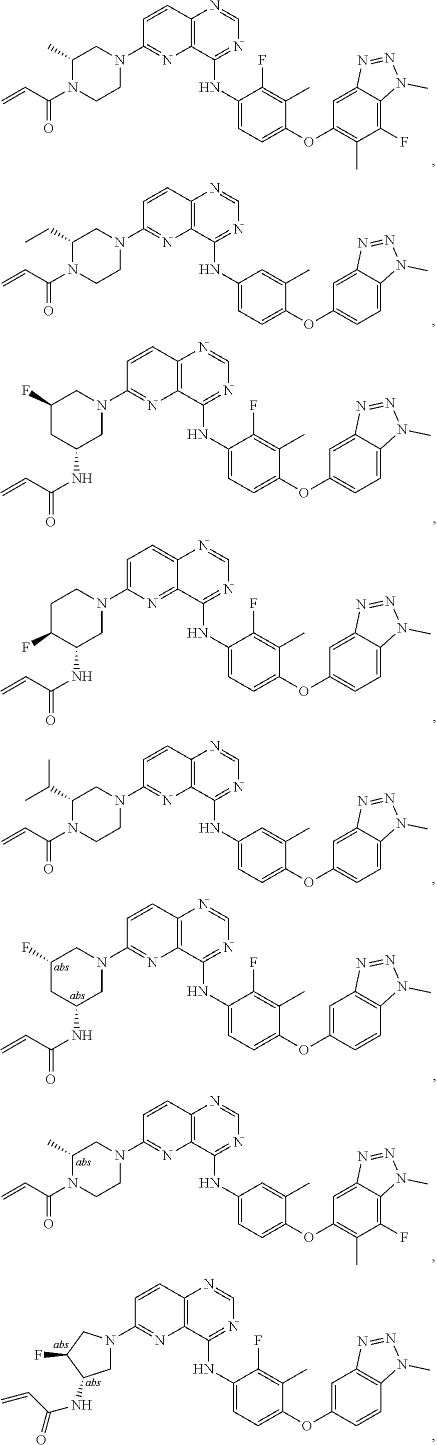

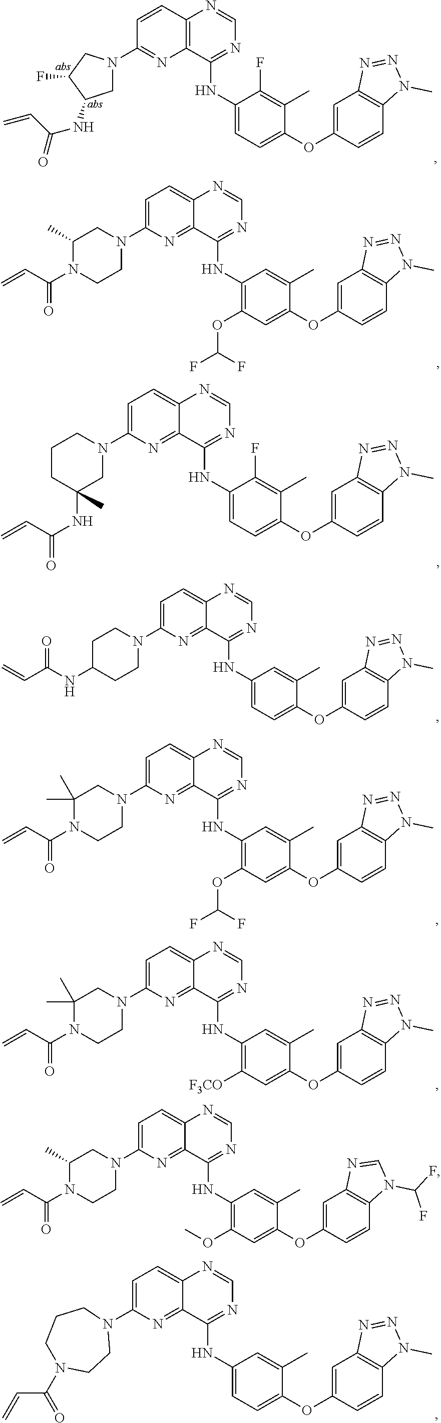

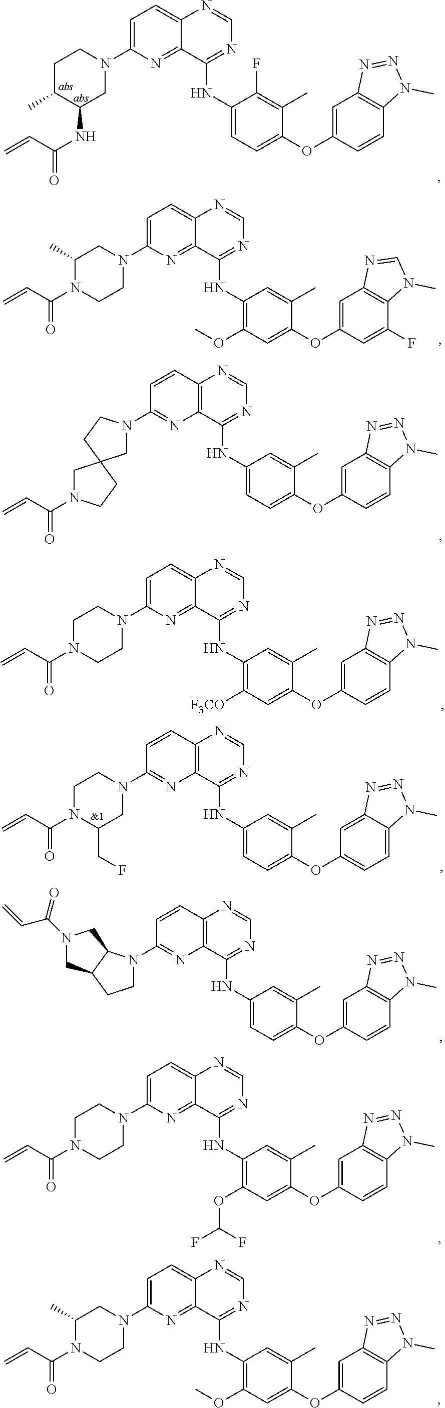

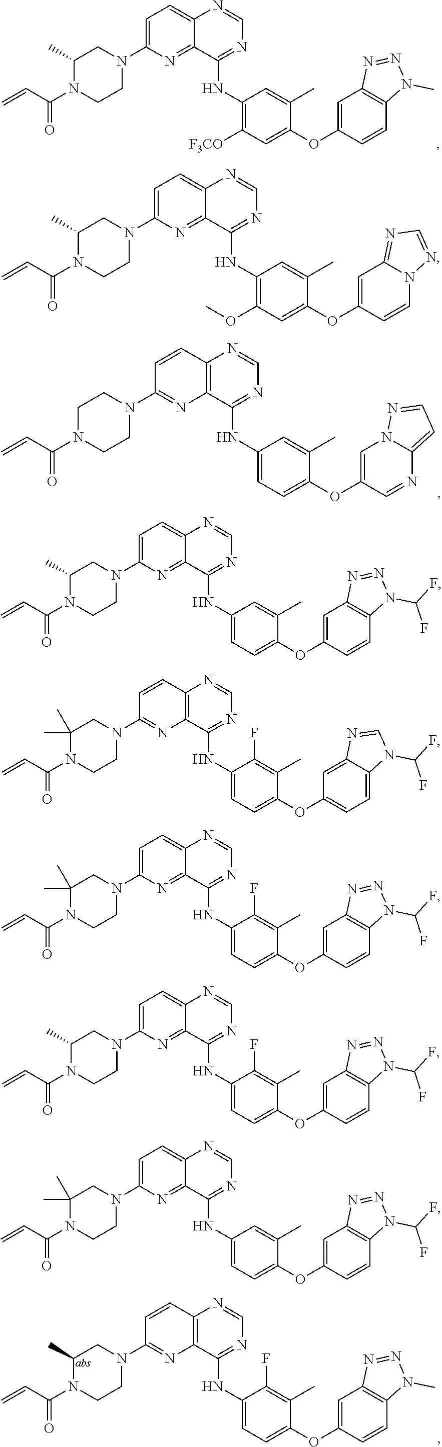

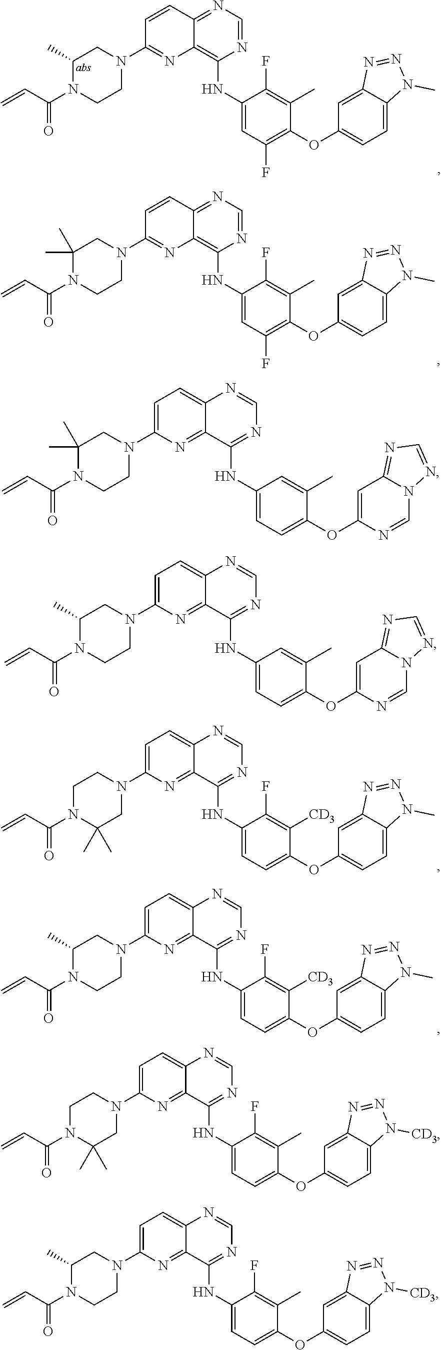

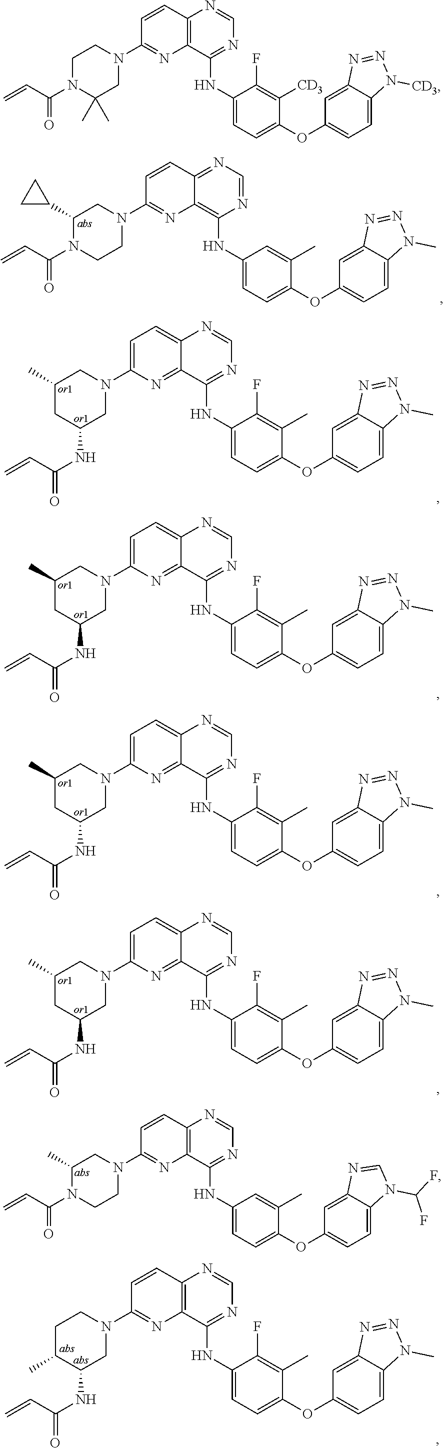

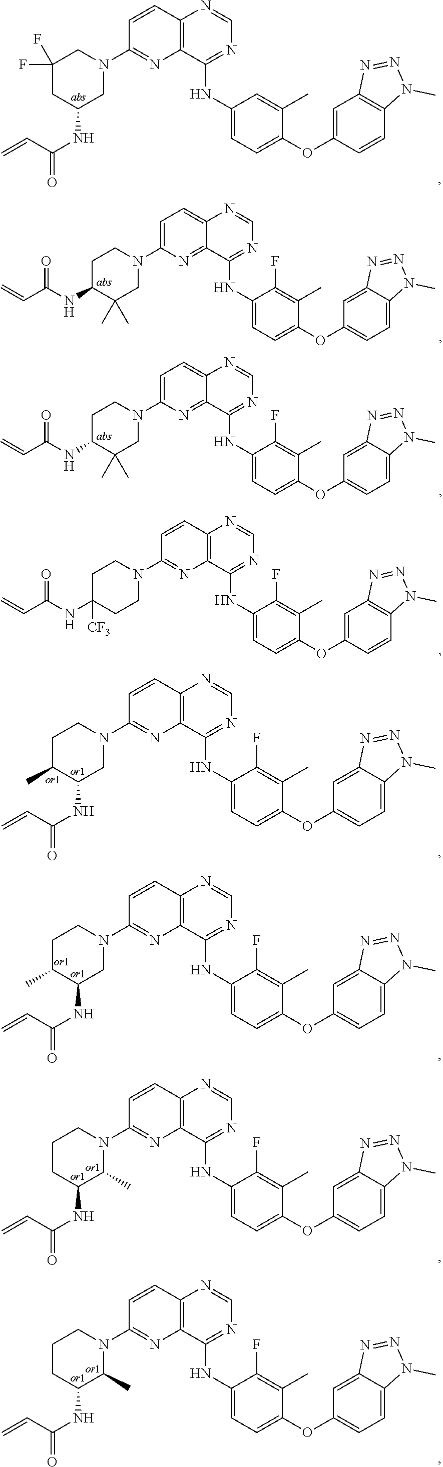

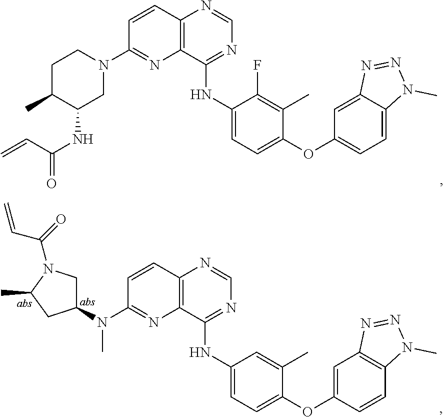

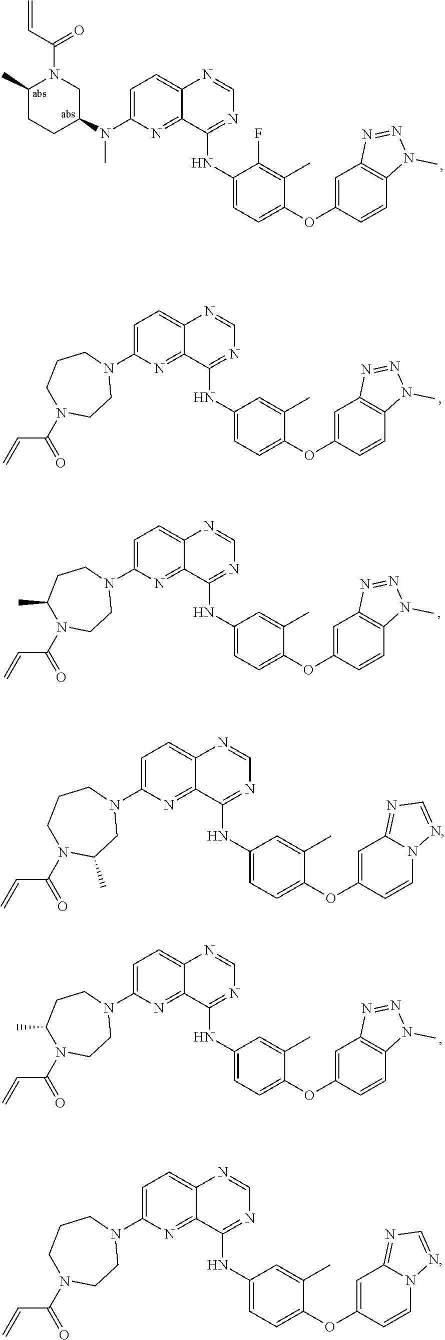

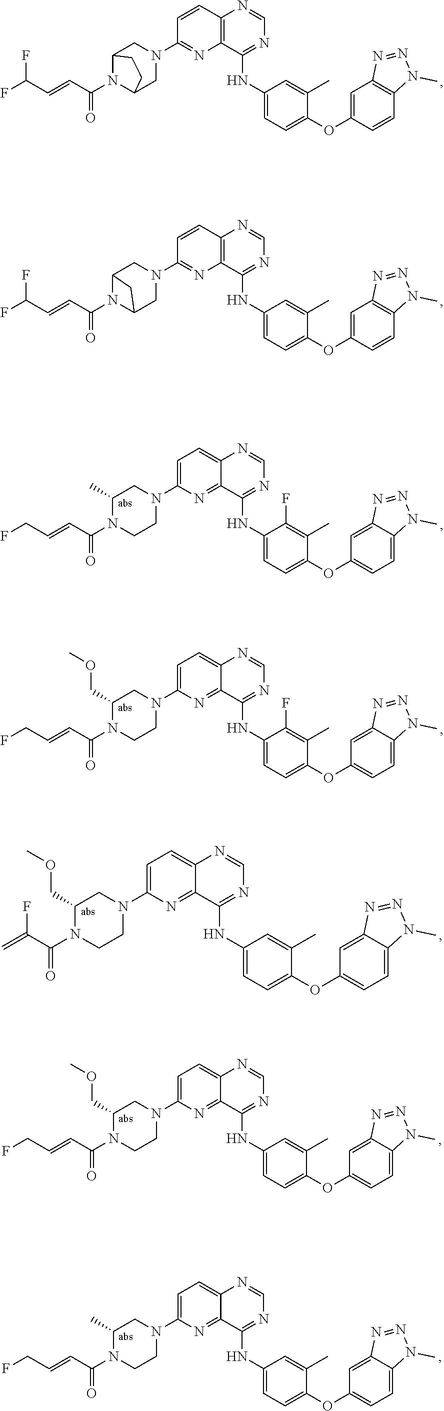

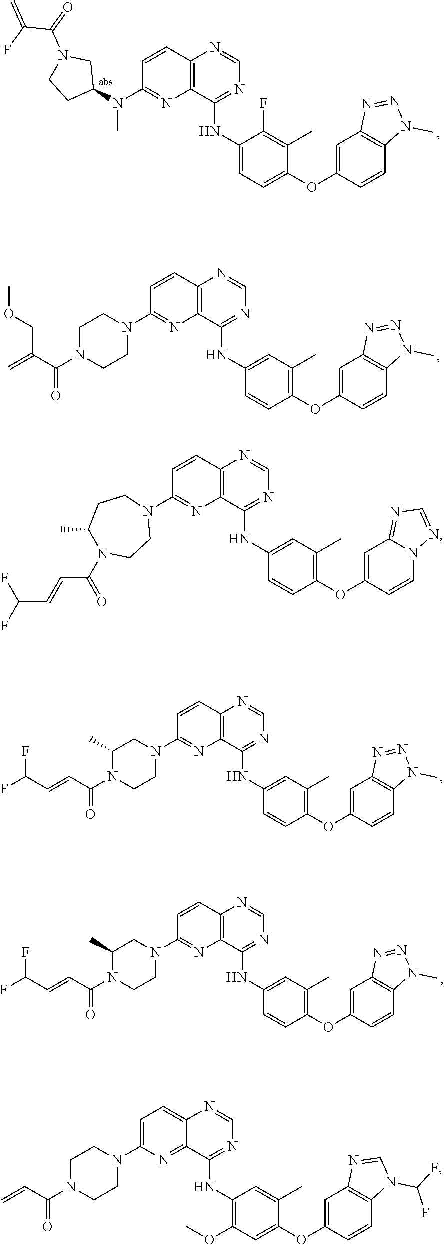

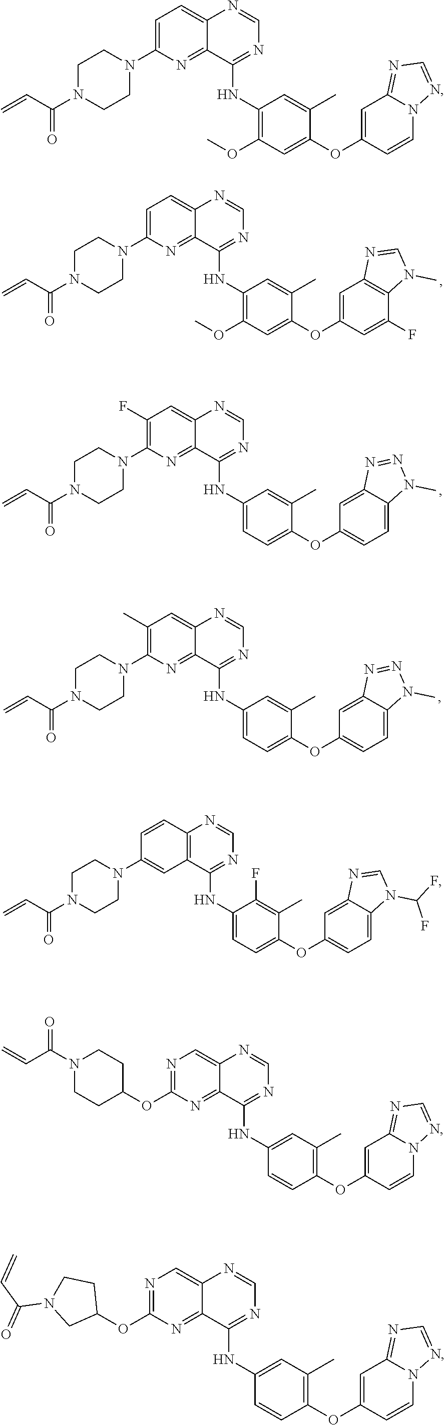

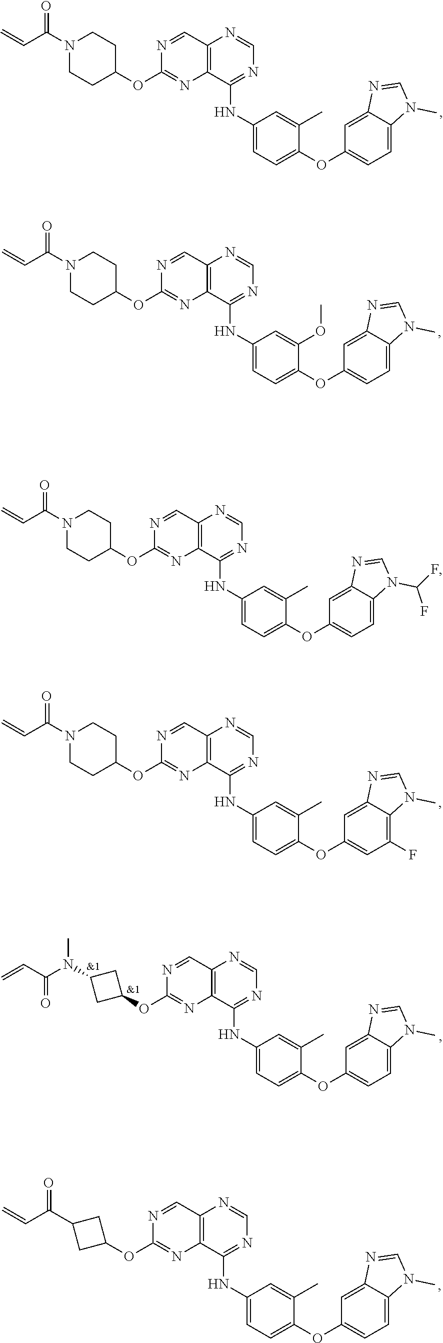

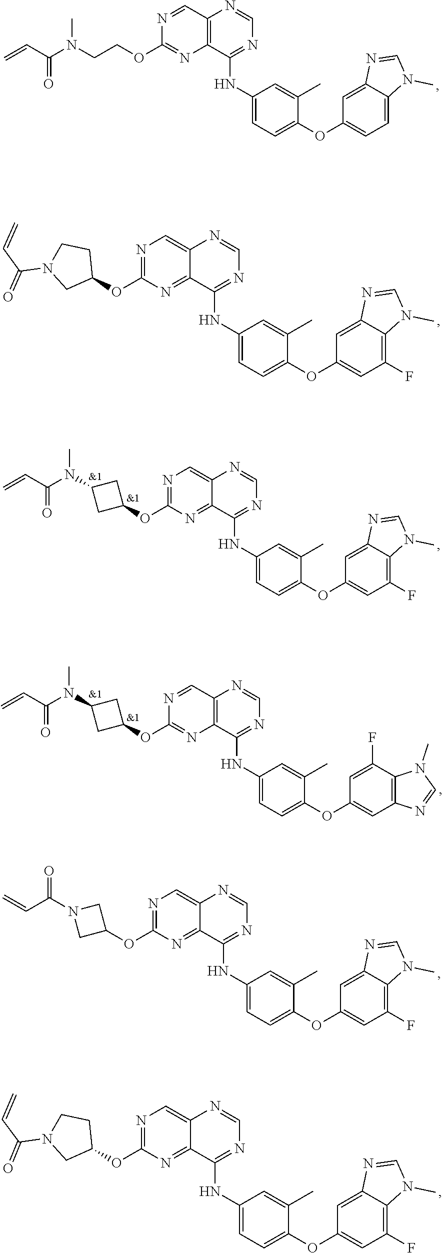

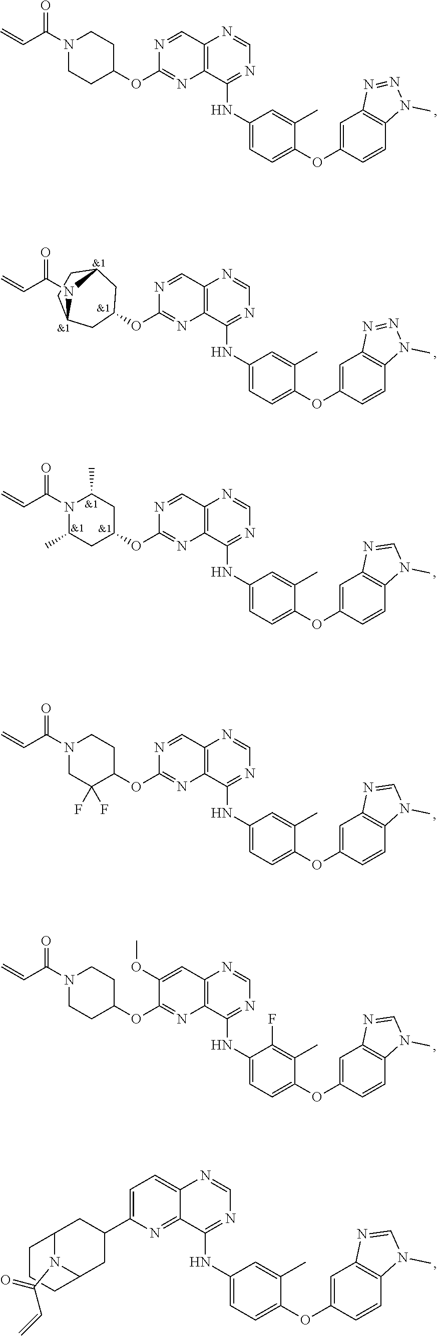

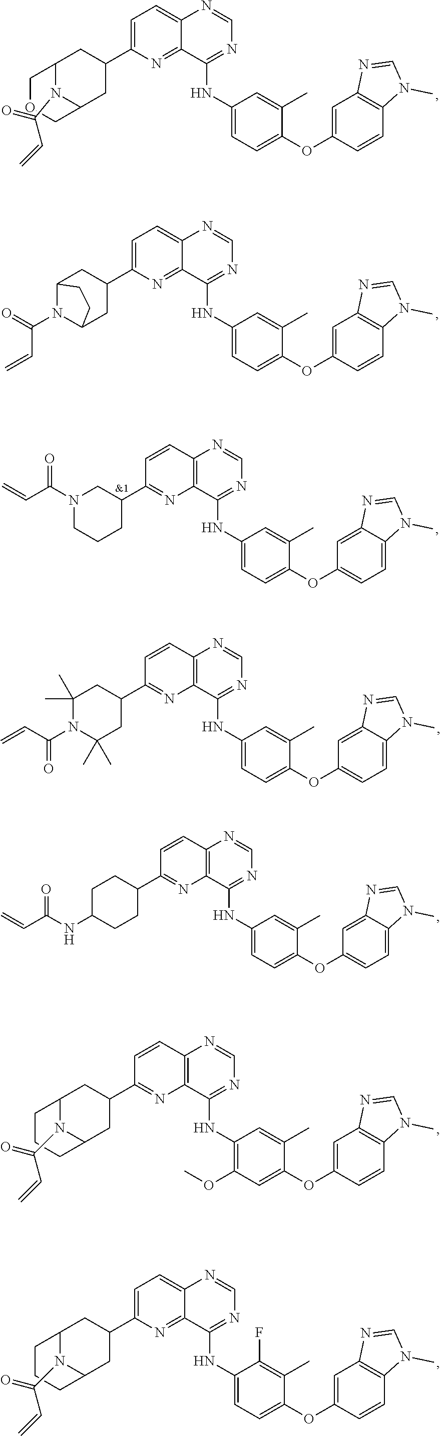

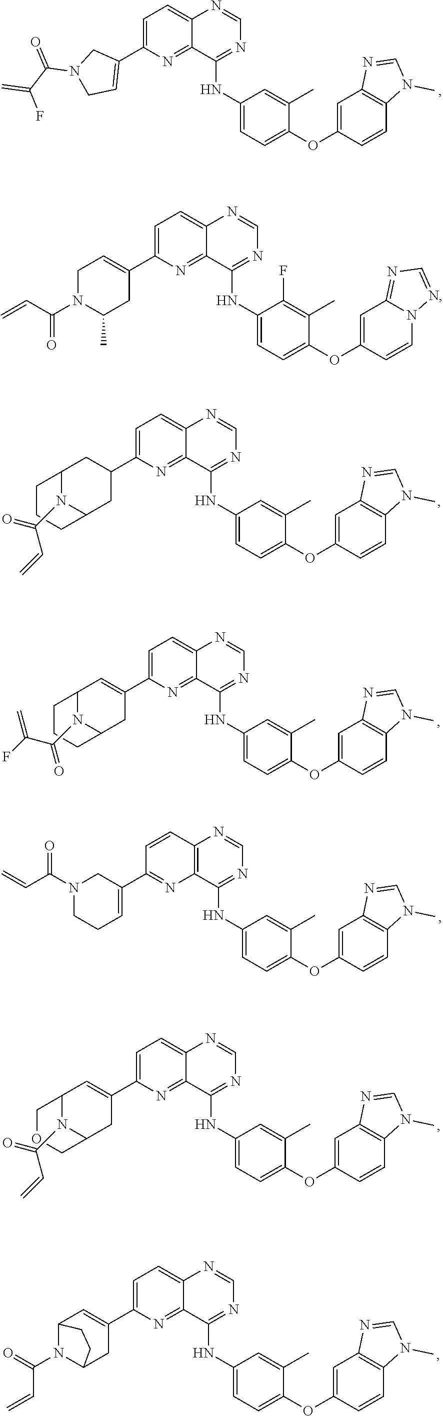

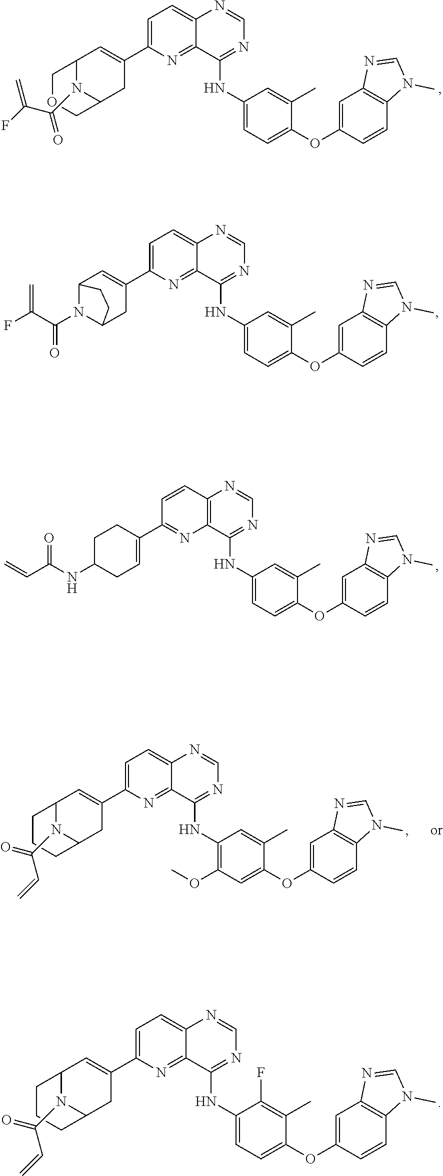

44. A pharmaceutical composition comprising the compound of claim 4 and an additional therapeutic agent.

45. (canceled)

46. A method of treating a disease associated with mutations in ErbB2, comprising: administering a compound of claim 4 to a subject in need thereof.

47.-51. (canceled)
